SEQUENCE LISTING (22 sequences)

SEQ ID NO: 1 - DNA, 21 bp, Artificial Sequence
  Synthesized Oligonucleotide.
ggcagaacugggcuuccugtt

SEQ ID NO: 2 - DNA, 21 bp, Artificial Sequence
  Synthesized Oligonucleotide.
caggaagcccaguucugcctt

SEQ ID NO: 3 - DNA, 21 bp, Artificial Sequence
  Synthesized Oligonucleotide.
agagcuggagcuggugcagtt

SEQ ID NO: 4 - DNA, 21 bp, Artificial Sequence
  Synthesized Oligonucleotide.
cugcaccagcuccagcucutt

SEQ ID NO: 5 - DNA, 21 bp, Artificial Sequence
  Synthesized Oligonucleotide.
gauggaguaugccgaggugtt

SEQ ID NO: 6 - DNA, 21 bp, Artificial Sequence
  Synthesized Oligonucleotide.
caccucggcauacuccauctt

SEQ ID NO: 7 - DNA, 21 bp, Artificial Sequence
  Synthesized Oligonucleotide.
cuuuggcuuuguuggggaatt

SEQ ID NO: 8 - DNA, 21 bp, Artificial Sequence
  Synthesized Oligonucleotide.
uuccccaacaaagccaaagtt

SEQ ID NO: 9 - DNA, 21 bp, Artificial Sequence
  Synthesized Oligonucleotide.
cgacaauuacccucaggcgtt

SEQ ID NO: 10 - DNA, 21 bp, Artificial Sequence
  Synthesized Oligonucleotide.
cgccugaggguaauugucgtt

SEQ ID NO: 11 - DNA, 21 bp, Artificial Sequence
  Synthesized Oligonucleotide.
gaugaagaaacggggggautt

SEQ ID NO: 12 - DNA, 21 bp, Artificial Sequence
  Synthesized Oligonucleotide.
auccccccguuucuucauctt

SEQ ID NO: 13 - DNA, 21 bp, Artificial Sequence
  Synthesized Oligonucleotide.
uucuccgaacgugucacgutt

SEQ ID NO: 14 - DNA, 21 bp, Artificial Sequence
  Synthesized Oligonucleotide.
acgugacacguucggagaatt

SEQ ID NO: 15 - DNA, 2067 bp, Homo sapiens
  misc_feature (20)..(20): n is a, c, g, or t
gaattccctcgcgctctggnccgggcgaatcgggntataggaagggccacacggatggaagtcctagtccgggtgctcacctcttgtggaacgtgcaaagcctgtcccaggacctctctacactctgggggtctctgcccaggcacgcttgctgcttccggacacagctgtgggcggagctagtaggggcgggctacgtgattgacacttctctcctcagacttcaagggctaccactggacccttcccctgtcttgaaccctgagccggcaccatgcacggacgcctgaaggtgaagacgtcagaagagcaggcggaggccaaaaggctagagcgagagcagaagctgaagctataccagtcagccacccaggccgtattccagaagcgccaggctggtgagctggatgagtccgtgctggaactgacaagccagattctgggagccaaccctgattttgccaccctctggaactgccgacgagaggtgctccagcagctggagactcagaagtctcctgaagagttggctgctctggtgaaggcagaactgggcttcctggagagctgcctgcgggtgaaccccaagtcttatggtacctggcaccaccgatgctggctgctaggccgcctgcctgagcccaactggacccgagagctggagctctgtgcccgtttcctggaggtggatgagcggaactttcactgctgggactatcggcggtttgtggccacacaggcagccgtgccccctgcagaagagctagccttcactgacagcctcatcacccgaaacttctccaactactcttcctggcattaccgctcctgtctcttgccccagttgcacccccagccggattctggaccacaggggcgcctccctgaggatgtgctgctcaaagagctggagctggtgcagaatgccttcttcactgaccccaatgaccagagtgcctggttttatcaccggtggctcctaggtcgagctgacccccaggatgcactgcgctgtctgcatgtgagccgggacgaggcctgtctgactgtctccttctctcggcccctcttagtgggctccaggatggagatcttgctgctcatggttgatgattctcccctgattgtggagtggaggaccccagatggcaggaaccggcccagccatgtctggctctgtgacctgcctgctgcctccctcaacgaccagttgccccaacatacatttcgcgtcatttggacagcaggcgatgtccagaaagaatgcgtgcttttaaaaggccgccaggagggctggtgccgggactccacgacagacgagcagctattcaggtgtgagctgtcagtggagaagtccacagtgctgcagtctgagctggaatcctgtaaggagctgcaggagctggagcctgagaataaatggtgcctgcttaccatcatcctgctgatgcgggcactggaccccctgctgtatgagaaggagaccctgcagtacttccagaccctcaaggccgtggaccccatgcgggcaacgtatctggatgacctgcgcagcaagttcttgctggagaatagcgtgctcaagatggagtatgccgaggtgcgtgtgctgcacctggctcacaaggatctgacagtgctctgccatctggaacagctgctcttggtcacccatcttgacttgtcacacaatcgcctccgaaccctgccacctgcactggctgccctgcgctgccttgaggtgctgcaggccagtgataatgcaatagagtccctggacggcgtcaccaacctaccccggctgcaggagctgctactgtgcaacaaccgcctccagcagcctgcagtgctccagcctcttgcctcctgccccaggctggtcctcctcaacctgcagggtaacccgctgtgccaagcggtgggcatcttggagcaactggctgaactgctgccttcagttagcagcgtcctcacctaagaggccctgccccctacccttgccctttaacttattgggactgaataaagaatggagaggcccctctcaggctaccaaaaaaaaaaaaa

SEQ ID NO: 16 - protein, 567 aa, Homo sapiens
MHGRLKVKTSEEQAEAKRLEREQKLKLYQSATQAVFQKRQAGELDESVLELTSQILGANPDFATLWNCRREVLQQLETQKSPEELAALVKAELGFLESCLRVNPKSYGTWHHRCWLLGRLPEPNWTRELELCARFLEVDERNFHCWDYRRFVATQAAVPPAEELAFTDSLITRNFSNYSSWHYRSCLLPQLHPQPDSGPQGRLPEDVLLKELELVQNAFFTDPNDQSAWFYHRWLLGRADPQDALRCLHVSRDEACLTVSFSRPLLVGSRMEILLLMVDDSPLIVEWRTPDGRNRPSHVWLCDLPAASLNDQLPQHTFRVIWTAGDVQKECVLLKGRQEGWCRDSTTDEQLFRCELSVEKSTVLQSELESCKELQELEPENKWCLLTIILLMRALDPLLYEKETLQYFQTLKAVDPMRATYLDDLRSKFLLENSVLKMEYAEVRVLHLAHKDLTVLCHLEQLLLVTHLDLSHNRLRTLPPALAALRCLEVLQASDNAIESLDGVTNLPRLQELLLCNNRLQQPAVLQPLASCPRLVLLNLQGNPLCQAVGILEQLAELLPSVSSVLT

SEQ ID NO: 17 - DNA, 1138 bp, Homo sapiens
  CDS (1)..(996)
atgggcactccacagaaggatgttattatcaagtcagatgcaccggacactttgttattggagaaacatgcagattatatcgcatcctatggctcaaagaaagatgattatgaatactgtatgtctgagtatttgagaatgagtggcatctattggggtctgacagtaatggatctcatgggacaacttcatcgcatgaatagagaagagattctggcatttattaagtcttgccaacatgaatgtggtggaataagtgctagtatcggacatgatcctcatcttttatacactcttagtgctgtccagattcttacgctgtatgacagtattaatgttattgacgtaaataaagttgtggaatatgttaaaggtctacagaaagaagatggttcttttgctggagatatttggggagaaattgacacaagattctctttttgtgcggtggcaactttcgctttgttggggaagcttgatgctattaatgtggaaaaggcaatcgaatttgttttatcctgtatgaactttgacggtggatttggttgcagaccaggttctgaatcccatgctgggcagatctattgttgcacaggatttctggctattacaagtcagttgcatcaagtaaattctgatttacttggctggtggctttgtgaacgacaattaccctcaggcgggctcaatggaaggccggagaagttaccagatgtatgctactcatggtgggtcctggcttccctaaagataattggaagacttcattggattgatagagagaaactgcgtaatttcattttagcatgtcaagatgaagaaacggggggatttgcagacaggccaggagatatggtggatccttttcataccttatttggaattgctggattgtcacttttgggagaagaacagattaaacctgttaatcctgtcttttgcatgcctgaagaagtgcttcagagagtgaatgttcagcctgagctagtgagctagattcattgaattgaaagttgcatagtatagttttgccattttaacatttctgtatttgaagtgcttatcgaatctaaaagtgactactgttaatattttgtatattgtgttaaattaattttaataaattatataattatat

SEQ ID NO: 18 - protein, 331 aa, Homo sapiens [sequence omitted]

SEQ ID NO: 19 - protein, 567 aa, Rattus norvegicus
MHGRLKVKTSEEQAEAKRLEREQKLKLYQSATQAVFQKRQAGELDESVLELTSQILGANPDFATLWNCRREVLQHLETEKSPEESAALVKAELGFLESCLRVNPKSYGTWHHRCWLLSRLPEPNWARELELCARFLEADERNFHCWDYRRFVAAQAAVAPAEELAFTDSLITRNFSNYSSWHYRSCLLPQLHPQPDSGPQGRLPENVLLKELELVQNAFFTDPNDQSAWFYHRWLLGRAEPHDVLCCVHVSREEACLSVCFSRPLTVGSRMGTLLLMVDEAPLSVEWRTPDGRNRPSHVWLCDLPAASLNDQLPQHTFRVIWTGSDSQKECVLLKDRPECWCRDSATDEQLFRCELSVEKSTVLQSELESCKELQELEPENKWCLLTIILLMRALDPLLYEKETLQYFSTLKAVDPMRAAYLDDLRSKFLLENSVLKMEYADVRVLHLAHKDLTVLCHLEQLLLVTHLDLSHNRLRALPPALAALRCLEVLQASDNALENVDGVANLPRLQELLLCNNRLQQSAAIQPLVSCPRLVLLNLQGNSLCQEEGIQERLAEMLPSVSSILT

SEQ ID NO: 20 - protein, 331 aa, Rattus norvegicus
MGTQQKDVTIKSDAPDTLLLEKHADYIASYGSKKDDYEYCMSEYLRMSGVYWGLTVMDLMGQLHRMNKEEILVFIKSCQHECGGVSASIGHDPHLLYTLSAVQILTLYDSIHVINVDKVVAYVQSLQKEDGSFAGDIWGEIDTRFSFCAVATLALLGKLDAINVEKAIEFVLSCMNFDGGFGCRPGSESHAGQIYCCTGFLAITSQLHQVNSDLLGWWLCERQLPSGGLNGRPEKLPDVCYSWWVLASLKIIGRLHWIDREKLRSFILACQDEETGGFADRPGDMVDPFHTLFGIAGLSLLGEEQIKPVSPVFCMPEEVLQRVNVQPELVS

SEQ ID NO: 21 - protein, 580 aa, Caenorhabditis elegans
MHFVKKVPTTEEEKAAKQKEHTKRSQQFLHVRDKIVAKREKGEYDDEILSLTQAILEKNADIYTFWNIRRTTIELRMEANEKVQQSADAEEEEKTKSSQKIENLLAGELFLSYECIKSNPKSYSAWYQRAWALQRQSAPDFKKELALCEKALQLDCRNFHCWDHRRIVARMAKRSEAEELEFSNKLINDNFSNYSAWHYRSIALKNIHRDEKTGAPKIDDELIASELQKVKNAFFMDAEDQSAWTYTRWLLEVGSGKEFLRPESHTPIELISASFHGNNTTLVFSRAVTIQFLLTFVDTENTTGWRAFSSTSPNPTSSRVWQYLSDTPLRVVTSNPTDLENISWTELNEQPYVNLDRLKTIYDVVEVPQPAYIGELLEDCKQLIELEPKNKWPLYMRTLVLLEYQPIKSYEEIIKNLENLSENLDPKRSELYKSLISRQNLNFSIREQFERILGPDTDWLTCRYSKLTSLEGVEYLAGFVGSADFSGNRLKEIQRIVLPNLKSLTINENPIESLPPSPCLSHLTFFSIAGTQIASVSAVMPFFQTIPSLDRLVFCETPLVEKTEELRAQLPGVRLIPHWL

SEQ ID NO: 22 - protein, 335 aa, Caenorhabditis elegans
MSFAGLLDFARKDVDLPQNSPNELLKDLHANFINQYEKNKNSYHYIMAEHLRVSGIYWCVNAMDLSKQLERMSTEEIVNYVLGCRNTDGGYGPAPGHDSHLLHTLCAVQTLIIFNSIEKADADTISEYVKGLQQEDGSFCGDLSGEVDTRFTLCSLATCHLLGRLSTLNIDSAVRFLMRCYNTDGGFGTRPGSESHSGQIYCCVGALAIAGRLDEIDRDRTAEWLAFRQCDSGGLNGRPEKLPDVCYSWWVLASLAILGRLNFIDSDAMKKFIYACQDDETGGFADRPGDCADPFHTVFGIAALSLFGDDTLESVDPIFCMTKRCLGDKQVEMYY

(19) United States
(12) Patent Application Publication
(10) Pub. No.: US 2004/0142888 A1
(43) Pub. Date: Jul. 22, 2004

(54) Modulators of RabGGT and methods of use thereof

(76) Inventors: Veeraswamy Manne, Philadelphia, PA (US); Mark Lynch, Madison, CT (US); Petra B. Ross-MacDonald, Pennington, NJ (US); Terry Stouch, West Windsor, NJ (US); Naomi Laing, Stoneham, MA (US); Pamela Carroll, Princeton, NJ (US); Kevin Fitzgerald, Lambertville, NJ (US); Louis J. Lombardo, Belle Mead, NJ (US); Michael R. Costa, San Francisco, CA (US); Mark E. Maxwell, San Francisco, CA (US); Rachel M. Kindt, San Carlos, CA (US); Mark R. Lackner, Brisbane, CA (US); Tak Hung, Foster City, CA (US); Carol L. O'Brian, Castro Valley, CA (US); Hai Guang Zhang, El Sobrante, CA (US); Katherine S. Brown, San Francisco, CA (US); Jae Moon Lee, Cupertino, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/638,225

(22) Filed: Aug. 7, 2003

Related U.S. Application Data
(60) Provisional application No. 60/476,722, filed on Jun. 6, 2003.
(60) Provisional application No. 60/401,604, filed on Aug. 7, 2002.

(51) Int. Cl. A61K 48/00; G01N 33/574; A61K 31/5513; A61K 31/47

(52) U.S. Cl. 514/44; 514/221; 514/310; 435/7.23

(57) ABSTRACT

The present invention provides methods for inducing apoptosis in a cell, the methods generally involving contacting the cell with an agent that reduces the level and/or activity of RabGGT. The present invention further provides methods for treating a disorder related to unwanted cell proliferation in an individual, the methods generally involving administering to the individual an agent that reduces the level and/or activity of RabGGT. The present invention further provides methods for reducing apoptosis in a cell, the methods generally involving increasing the level and/or activity of RabGGT in the cell. The present invention further provides methods for treating disorders associated with excessive apoptosis. The present invention further provides methods for identifying a cell that is amenable to treatment with the methods of the present invention. The present invention further provides methods for modulating a binding event between RabGGT and a RabGGT interacting protein. The present invention further provides a 3-dimensional structure of RabGGT, and methods of use of the structure to identify compounds that modulate RabGGT activity.

This application claims benefit to provisional application U.S. Serial No. 60/401,604 filed Aug. 7, 2002; and U.S. Serial No. 60/476,722 filed Jun. 6, 2003; under 35 U.S.C. 119(e). The entire teachings of the referenced applications are incorporated herein by reference.

### FIELD OF THE INVENTION

The present invention is in the field of modulators of enzyme activity, in particular modulators of Rab-geranylgeranyl transferase, and their use in controlling cell proliferation.

### BACKGROUND OF THE INVENTION

Apoptosis is a coordinated program for induction of-a cell suicide process. Conserved components of the apoptotic pathway such as cytochrome c, the Bcl-2 family, Apaf-1, and the caspases have been identified in most eukaryotic systems. Cytochrome c release from the mitochondria via a permeability transition pore is a key trigger for apoptosis. The Bcl-2 family are highly conserved mitochondrial proteins that can act to enhance (bax, bid, bak, bad, bcl-xs) or prevent (Bcl-2, bcl-xl) apoptosis; they may effect formation of the pore. Apaf-1 is a cytoplasmic protein that is triggered by cytochrome C to activate caspase 9, which then cleaves and activates caspase 3. Caspases are proteases that act in a cascade and cleave multiple substrates, resulting in the morphological changes associated with apoptosis. Examples of changes include chromatin condensation and aggregation to the nuclear margin, cytoplasmic shrinkage, DNA fragmentation, and the packaging of cellular components into membrane bound compartments. Such specific changes distinguish apoptotic death, which may affect single cells in otherwise healthy tissue, from necrosis, in which groups of cells lyse.

Apoptosis can be activated by a number of intrinsic or extrinsic signals. These signals include the following: mild physical signals, such as ionization radiation, ultraviolet radiation, or hyperthermia; low to medium doses of toxic compounds, such as azides or hydrogen peroxides; chemotherapeutic drugs, such as etoposides and teniposides, cytokines such as tumour necrosis factors and transforming growth factors; infection with human immunodeficiency virus (HIV); and stimulation of T-cell receptors. Various pathological processes, such as hormone deprivation, growth factor deprivation, thermal stress and metabolic stress, induce apoptosis. (Wyllie, A. H., in Bowen and Lockshin (eds.) Cell Death in Biology and Pathology (Chapman and Hall, 1981), at 9-34).

Unregulated apoptosis can cause, or be associated with, disease. An understanding of how apoptosis can be regulated by drugs is becoming of increasing importance to the pharmaceutical industry (Kinloch et al., 1999, Trends in Pharmacological Science 20:35; Nicholson, 2000, Nature 407:810). For example, unregulated apoptosis is involved in diseases such as cancer, heart disease, neurodegenerative disorders, autoimrnmune disorders, and viral and bacterial infections. Cancer, for example, not only triggers cells to proliferate but also blocks apoptosis. Cancer is partly a failure of apoptosis in the sense that the signal(s) for the cells to kill themselves by apoptosis are blocked. Thus, inducing apoptosis may be a therapeutic strategy for the treatment of cancer.

In heart disease, damage caused by trauma (e.g, resulting in shock), and cardiac cells can be induced to undergo apoptosis. For example, cells deprived of oxygen after a heart attack release signals that induce apoptosis in cells in the heart. Apoptosis may also be involved in the destruction of neurons in people afflicted by strokes or neurodegenerative diseases such as Alzheimer's disease, Parkinson's disease, and amyotrophic lateral sclerosis (ALS). There is also evidence suggesting that ischemia can kill neurons by inducing apoptosis. It has been shown that neurons that are resistant to apoptosis are also resistant to ischemic damage, thus, inhibition of apoptosis may be a therapeutic strategy for the treatment of neurodegenerative or cardiovascular disorders, e.g., stroke.

Rab-geranylgeranyl transferase (RabGGT; GGTII) is a protein-prenyl transferase enzyme composed of a single alpha and beta subunit. These subunits have limited homology to the alpha subunit shared by Farnesyl transferase (FT) and geranylgeranyl transferase I (GGTI), and to the beta subunits that are distinct to each of those enzymes. RabGGT is unique among prenlyation enzymes in requiring specific accessory proteins known as Rab escort proteins (REPs) for their prenylation function. However the three prenylating enzymes are similar in the structure of their active sites and in their mechanism of substrate modification. The only RabGGT substrates identified to date are a large family of Ras-related proteins called Rabs. Rab proteins are monomeric GTPases that regulate intracellular membrane traffic. RabGGT acts on the Rab proteins to attach a geranylgeranyl moiety to one or two cysteine residues at the C-terminus of the protein. This prenylation event is important for the subcellular targeting of Rabs to membranes.

There is an ongoing need in the art for agents and methods of modulating cell proliferation. The present invention addresses this need.

Literature

Hengartner (2000) Nature 407:770; Long et al. (2002) Nature 419:645; Seabra et al., 2002, Trends in Molecular Medicine 8:23; Detter et al., 2000, Proc. Natl. Acad. Sci. USA 97:4144; Ren et al., 1997, Biochem. Pharmacol. 54:113; J. C. Reed, Nature Reviews Drug Discovery: 1 pp111-121; Kinloch et al., 1999, Trends in Pharmacological Science 20:35; Nicholson (2000) Nature 407:810; Thoma et al. (2000) Biochem. 39:12043-12052; Coxon et al. (2001) J. Biol. Chem. 276:48213-48222; Rose et al. (2001) Cancer Res. 61:7505-7517; Hunt et al. (2000) J. Med. Chem. 43:3587; Pylypenko et al. (2003) Molec. Cell 11:483-494.

### SUMMARY OF THE INVENTION

The present invention provides methods for inducing apoptosis in a cell, the methods generally involving contacting the cell with an agent that reduces the level and/or activity of RabGGT. The present invention further provides methods for treating a disorder related to unwanted cell proliferation in an individual, the methods generally involving administering to the individual an agent that reduces the level and/or activity of RabGGT. The present invention further provides methods for reducing apoptosis in a cell, the methods generally involving increasing the level and/or activity of RabGGT in the cell. The present invention further provides methods for treating disorders associated with excessive apoptosis. The present invention further provides methods for identifying a cell that is amenable to treatment with the methods of the present invention. The present invention further provides methods for modulating a binding event between RabGGT and a RabGGT interacting protein. The present invention further provides a 3-dimensional structure of RabGGT, and methods of use of the structure to identify compounds that modulate RabGGT activity.

The invention also provides a computer for producing a three-dimensional representation of a molecule or molecular complex, wherein said molecule or molecular complex comprises the structural coorrdinates of the model RabGGT alpha or beta subunit in accordance with Table 11 or 12, or a three-dimensional representation of a homologue of said molecule or molecular complex, wherein said homologue comprises backbone atoms that have a root mean square deviation from the backbone atoms of not more than about 4.0, 3.0. 2.0, 1.0, 0.9, 0.8, 0.7, 0.6, 0.5, 0.4, 0.3, 0.2, or 0.1 Angstroms, wherein said computer comprises: A machine-readable data storage medium, comprising a data storage material encoded with machine readable data, wherein the data is defined by the set of structure coordinates of the model RabGGT alpha or beta subunit according to Table 11 or 12, or a homologue of said model, wherein said homologue comprises backbone atoms that have a root mean square deviation from the backbone atoms of not more than about 4.0, 3.0. 2.0, 1.0, 0.9, 0.8, 0.7, 0.6, 0.5, 0.4, 0.3, 0.2, or 0.1 Angstroms; a working memory for storing instructions for processing said machine-readable data; a central-processing unit coupled to said working memory and to said machine-readable data storage medium for processing said machine readable data into said three-dimensional representation; and a display coupled to said central-processing unit for displaying said three-dimensional representation.

The invention also provides a machine readable storage medium which comprises the structure coordinates of RabGGT alpha or beta subunit, including all or any parts of conserved binding site regions. Such storage medium encoded with these data are capable of displaying on a computer screen or similar viewing device, a three-dimensional graphical representation of a molecule or molecular complex which comprises said regions or similarly shaped homologous regions.

The invention also provides methods for designing, evaluating and identifying compounds which bind to all or parts of the aforementioned regions. The methods include three dimensional model building (homology modeling) and methods of computer assisted-drug design which can be used to identify compounds which bind or modulate the forementioned regions of the RabGGT alpha or beta subunit polypeptide. Such compounds are potential inhibitors of RabGGT alpha or beta subunit or its homologues.

The invention also provides a machine-readable data storage medium, comprising a data storage material encoded with machine readable data, wherein the data is defined by the structure coordinates of the model RabGGT alpha or beta subunit according to Table 11 or 12 or a homologue of said model, wherein said homologue comprises any kind of surrogate atoms that have a root mean square deviation from the backbone atoms of the complex of not more than about 4.0, 3.0. 2.0, 1.0, 0.9, 0.8, 0.7, 0.6, 0.5, 0.4, 0.3, 0.2, 0.1, or less Angstroms.

The invention also provides a machine-readable data storage medium, comprising a data storage material encoded with machine readable data, wherein the data is defined by the structure coordinates of the model RabGGT alpha or beta subunit according to Table 11 or 12 or a homologue of said model, wherein said homologue comprises any kind of surrogate atoms that have a root mean square deviation from the backbone atoms of the complex of not more than about 4.0, 3.0. 2.0, 1.0, 0.9, 0.8, 0.7, 0.6, 0.5, 0.4, 0.3, 0.2, 0.1, or less Angstroms

The invention also provides a model comprising all or any part of the model defined by structure coordinates of RabGGT alpha or beta subunit according to Table 11 or 12, or a mutant or homologue of said molecule or molecular complex.

The invention also provides a method for identifying a mutant of RabGGT alpha or beta subunit with altered biological properties, function, or reactivity, the method comprising one or more of the following steps:

(a) use of the model or a homologue of said model according to Table 11 or 12, for the design of protein mutants with altered biological function or properties which exhibit any combination of therapeutic effects described herein; and/or (b) use of the model or a homologue of said model, for the design of a protein with mutations in the active site region according to Table 11 or 12 with altered biological function or properties which exhibit any combination of therapeutic effects described herein.

The method also relates to a method for identifying modulators of RabGGT alpha or beta subunit biological properties, function, or reactivity, the method comprising the step of modeling test compounds that fit spatially into the active site region defined by all or any portion of residues that embody this domain within the three-dimensional structural model according to Table 11 or 12, or using a homologue or portion thereof, or analogue in which the original C, N, and O atoms have been replaced with other elements

The invention also provides methods for designing, evaluating and identifying compounds which bind to all or parts of the aforementioned regions. The methods include three dimensional model building (homology modeling) and methods of computer assisted-drug design which can be used to identify compounds which bind or modulate the forementioned regions of the RabGGT alpha or beta subunit polypeptide. Such compounds are potential inhibitors of RabGGT alpha or beta subunit or its homologues.

The invention also relates to a method of using said structure coordinates as set forth in Table 11 or 12 to identify structural and chemical features of RabGGT alpha or beta subunit; employing identified structural or chemical features to design or select compounds as potential RabGGT alpha or beta subunit modulators; employing the three-dimensional structural model to design or select compounds as potential RabGGT alpha or beta subunit modulators; synthesizing the potential RabGGT alpha or beta subunit modulators; screening the potential RabGGT alpha or beta subunit modulators in an assay characterized by binding of a protein to the RabGGT alpha or beta subunit. The invention also relates to said method wherein the potential RabGGT alpha or beta subunit modulator is selected from a database. The invention further relates to said method wherein the potential RabGGT alpha or beta subunit modulator is designed de novo. The invention further relates to a method wherein the potential RabGGT alpha or beta subunit modulator is designed from a known modulator of activity.

### BRIEF DESCRIPTION OF THE DRAWINGS

FIG. 1 provides a graphical display of data on the effects of compound treatments upon levels of apoptosis in the worm germline (The percentage of germline arms examined that contained greater than 2 apoptotic corpses is displayed. Compound treatments are shown on the X axis);

FIG. 2 provides a graphical display of data on the effects of compound treatments upon levels of apoptosis in the germline of apoptosis-defective mutant worms (Average number of apoptotic corpses per germline arm in worms treated with compound 7B or vehicle. Worm genotype is displayed on the X-axis. The error bars shown standard deviation.);

FIG. 3 provides a graphical display of data on the effects of RNAi treatments against RabGGT subunits upon levels of apoptosis in the worm germline (The percentage of germline arms that contained greater than 2 apoptotic corpses is displayed. RNAi treatments are shown on the X axis.);

FIG. 4 provides a graphical display of data on the effects of treatment with compound and/or RNAi against RabGGT subunit alpha upon levels of apoptosis in the worm germline (The percentage of germline arms examined that contained either less than three, three or four, or greater than four apoptotic corpses is displayed. Treatments are shown on the X axis.);

FIG. 5 provides a graphical display of data on the effects of treatment with RNAi against RabGGT alpha subunit upon levels of apoptosis in the germline of Wild Type or compound 7B-resistant mutant worms (The percentage of germline arms in wild-type or mutant worms that contained greater than two apoptotic corpses is displayed. Treatments are shown on the X axis.);

FIG. 6 provides a graphical display of data on the effects of treatment with RNAi against RabGGT subunits upon levels of proliferation in human cells (3H-uptake by HCT116 cells as percentage of control treatment. Treatments are shown on the X-axis.);

FIG. 7 provides a graphical display of results obtained by non-linear regression analysis of data obtained for compound 7B in a RabGGT inhibition assay (Results obtained by non-linear regression analysis of data obtained for compound 7B.);

FIG. 8a provides a graphical display of the data on RabGGT inhibition and apoptotic activity for the benzodiazepine class of compounds (Data from the benzodiazepine class of compounds: The IC90 for RabGGT inhibition in nanomoles is shown on the Y axis and the minimum concentration required for induce 50% apoptosis in an HCT116 cell culture is shown on the X axis.);

FIG. 8b provides a graphical display of the data on RabGGT inhibition and apoptotic activity for the tetrahydroquinolone class of compounds (Data from the tetrahydroquinolone class of compounds: The IC90 for RabGGT inhibition in nanomoles is shown on the Y axis and the minimum concentration required for induce 50% apoptosis in an HCT116 cell culture is shown on the X axis.);

FIG. 8c provides a graphical display of data on RabGGT inhibition and apoptotic activity for compounds 7A-7Q (Data from compounds 7A through 7Q. Compounds 7R, 7S, and 7T are represented in FIG. 9b, and have been omitted from this figure for graphical clarity rather than because they alter the trend of the observations. The IC90 for RabGGT inhibition in nanomoles is shown on the Y axis and the minimum concentration required for induce 50% apoptosis in an HCT116 cell culture is shown on the X axis.);

FIG. 9 provides a graphical display of data on FT inhibition and apoptotic activity for compounds 7A-7T (Data for compounds 7A through 7T. The IC50 for FT inhibition in nanomoles is shown on the Y axis and the minimum concentration required for induce 50% apoptosis in an HCT116 cell culture is shown on the X axis.);

FIG. 10 provides a superposition of the homology model of the H. sapiens RabGGT protein on the crystal structure of the rat RabGGT protein (Superposition of the homology model of the human RabGGT protein (dark) on the crystal of the rat RabGGT protein. The atom of zinc found in the binding site of the rat protein is shown as a white sphere.);

FIG. 11a provides free energy plots for the modeled human RabGGT alpha subunit and for the crystal structure of the rat RabGGT alpha subunit (Energy plots for the model of H. sapiens RabGGT alpha chain (dotted line), and for the crystal structure of the R. norvegicus RabGGT alpha chain (solid line)).

FIG. 11b provides free energy plots for the modeled human RabGGT beta subunit and for the crystal structure of the rat RabGGT beta subunit (Energy plots for the model of H. sapiens RabGGT beta chain (dotted line), and for crystal structure of the R. norvegicus RabGGT beta chain (solid line)).

FIG. 12 provides a superposition of the homology model of the C. elegans RabGGT protein on the crystal structure of the rat RabGGT protein (Superposition of the homology model of the C. elegans RabGGT protein (dark) on the crystal of the rat RabGGT protein. The atom of zinc found in the binding site of the rat protein is shown as a white sphere.);

FIG. 13a provides free energy plots for the modeled C. elegans RabGGT alpha subunit and for the crystal structure of the rat RabGGT alpha subunit (Energy plots for the model of C. elegans RabGGT alpha chain (dotted line), and for the crystal structure of the R. norvegicus RabGGT alpha chain (solid line)).

FIG. 13b provides free energy plots for the modeled C. elegans RabGGT beta subunit and for the crystal structure of the rat RabGGT beta subunit (Energy plots for the model of C. elegans RabGGT beta chain (dotted line), and for the crystal structure of the R. norvegicus RabGGT beta chain (solid line)).

FIG. 14a provides a depiction of the binding site in the crystal structure of the rat RabGGT enzyme (Binding pocket from the crystal structure of rat RabGGT. The white sphere denotes the bound atom of zinc.);

FIG. 14b provides a depiction of the superimposition of the binding site in the crystal structure of the rat RabGGT enzyme upon the binding site in the model of the human RabGGT enzyme (Superposition of the residues within 5 Angstrom of the binding site in the homology model of the H. sapiens RabGGT protein (dark) on the crystal structure of the homologous residues of the rat protein. The atom of zinc found in the binding site of the rat protein is shown as a white sphere.);

FIG. 14c provides a depiction of the superimposition of the binding site in the crystal structure of the rat RabGGT enzyme upon the binding site in the model of the C. elegans RabGGT enzyme (Superposition of the residues within 5 Angstrom of the binding site in the homology model of the C. elegans RabGGT protein (dark) on the crystal structure of the homologous residues of the rat protein. The atom of zinc found in the binding site of the rat protein is shown as a white sphere).

FIG. 15A depicts binding of compound 7H docked into the putative binding site of RabGGT.

FIG. 15B depicts the binding site of the crystal structure of the complex between farnesyl transferase and the FT inhibitor U66.

FIG. 16A-B show the polynucleotide sequence (SEQ ID NO:15) and deduced amino acid sequence (SEQ ID NO:16) of the human RabGGT alpha subunit. The standard one-letter abbreviation for amino acids is used to illustrate the deduced amino acid sequence.

FIG. 17 show the polynucleotide sequence (SEQ ID NO:17) and deduced amino acid sequence (SEQ ID NO:18) of the human RabGGT beta subunit. The standard one-letter abbreviation for amino acids is used to illustrate the deduced amino acid sequence.

### DEFINITIONS

As used herein, the term “disorder associated with undesired or uncontrolled cell proliferation” is any disorder that results from undesired or uncontrolled cell proliferation, and/or that is amenable to treatment by inducing apoptosis in the cell, such disorders including, but not limited to, cancer, viral infection, disorders associated with excessive or unwanted angiogenesis, and the like.

As used herein, the term “disorder associated with excessive apoptosis” is any disorder that results from an excessive amount of apoptosis, such disorders including, but not limited to, sepsis, atherosclerosis, muscle cachexia, ischemia/reperfusion injury, neurodegenerative disorders, and myocardial infarction.

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, e.g., causing regression of the disease, e.g., to completely or partially remove symptoms of the disease.

The term “biological sample” encompasses a variety of sample types obtained from an organism and can be used in a diagnostic or monitoring assay. The term encompasses blood and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses a clinical sample, and also includes cells in cell culture, cell supernatants, cell lysates, serum, plasma, biological fluids, and tissue samples.

The terms “cancer”, “neoplasm”, “tumor”, and “carcinoma”, are used interchangeably herein to refer to cells which exhibit relatively autonomous growth, so that they exhibit an aberrant growth phenotype characterized by a significant loss of control of cell proliferation. Cancerous cells can be benign or malignant.

By “individual” or “host” or “subject” or “patient” is meant any mammalian subject for whom diagnosis, treatment, or therapy is desired, particularly humans. Other subjects may include cattle, dogs, cats, guinea pigs, rabbits, rats, mice, horses, and so on.

The term “binds specifically,” in the context of antibody binding, refers to high avidity and/or high affinity binding of an antibody to a specific polypeptide i.e., epitope of a polypeptide, e.g., RabGGT. For example, antibody binding to an epitope on a specific RabGGT polypeptide or fragment thereof is stronger than binding of the same antibody to any other epitope, particularly those which may be present in molecules in association with, or in the same sample, as the specific polypeptide of interest, e.g., binds more strongly to a specific RabGGT epitope than to a different RabGGT epitope so that by adjusting binding conditions the antibody binds almost exclusively to the specific RabGGT epitope and not to any other RabGGT epitope, and not to any other RabGGT polypeptide (or fragment) or any other polypeptide which does not comprise the epitope. Antibodies which bind specifically to a polypeptide may be capable of binding other polypeptides at a weak, yet detectable, level (e.g., 10% or less of the binding shown to the polypeptide of interest). Such weak binding, or background binding, is readily discernible from the specific antibody binding to a subject polypeptide, e.g. by use of appropriate controls. In general, specific antibodies bind to a given polypeptide with a binding affinity of 10⁻⁷ M or more, e.g., 10⁻⁸ M or more (e.g., 10⁻⁹ M, 10⁻¹⁰ M, 10⁻¹¹ M, etc.). In general, an antibody with a binding affinity of 10⁻⁶ M or less is not useful in that it will not bind an antigen at a detectable level using conventional methodology currently used.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “an agent” includes a plurality of such agents and reference to “the inhibitor” includes reference to one or more inhibitors and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention.

Further, the dates of publication provided may be different from the actual publication dates which may need to be independently confirmed.

### DETAILED DESCRIPTION OF THE INVENTION

The present invention provides methods for inducing apoptosis in a cell, the methods generally involving contacting the cell with an agent that reduces the level and/or activity of RabGGT. The present invention further provides methods for treating a disorder related to unwanted cell proliferation in an individual, the methods generally involving administering to the individual an agent that reduces the level and/or activity of RabGGT. The present invention further provides methods for reducing apoptosis in a cell, the methods generally involving increasing the level and/or activity of RabGGT in the cell. The present invention further provides methods for treating disorders associated with excessive apoptosis. The present invention further provides methods for identifying a cell that is amenable to treatment with the methods of the present invention. The present invention further provides methods for modulating a binding event between RabGGT and a RabGGT interacting protein. The present invention further provides a 3-dimensional structure of RabGGT, and methods of use of the structure to identify compounds that bind specifically to RabGGT.

The present invention is based in part on the observation that inhibitors of RabG GT levels and/or activity induce apoptosis and reduce cell proliferation. As discussed in the Examples section, inhibitors of RabGGT induced tumor regression in a human tumor xenograft model, and induced apoptosis of cells expressing RabGGT in cell cultures in vitro and in vivo.

### Treatment Methods

In some embodiments, the invention provides methods for inducing apoptosis in a cell and/or inhibiting proliferation of the cell. The methods generally involve contacting a cell with an effective amount of an agent that inhibits a level and/or activity of RabGGT or a RabGGT/REP complex. The invention also provides methods of treating a disorder amenable to treatment by inducing apoptosis and/or inhibiting cell proliferation, the methods generally involving administering an effective amount of an agent that inhibits a level and/or activity of RabGGT or a RabGGT/REP complex in a cell in the individual.

As used herein, the term “RabGGT” refers to a protein that includes a RabGGT α subunit and a RabGGT β subunit. As used herein, an “agent that reduces the level of a RabGGT protein” includes an agent that reduces the level of a RabGGT α subunit (and does not reduce the level of a RabGGT β subunit), an agent that reduces the level of a RabGGT β subunit (and does not reduce the level of a RabGGT β subunit), and an agent that reduces the level of both a RabGGT α subunit and a RabGGT β subunit. As used herein, an “agent that reduces the level of a RabGGT mRNA” includes an agent that reduces the level of an mRNA encoding a RabGGT α subunit (and does not reduce the level of an mRNA encoding a RabGGT β subunit), an agent that reduces the level of an mRNA encoding a RabGGT β subunit (and does not reduce the level of an mRNA encoding a RabGGT β subunit), and an agent that reduces the level of both an mRNA encoding a RabGGT α subunit and an mRNA encoding a RabGGT β subunit.

An “effective amount” of an agent that inhibits a level and/or activity of RabGGT is an amount that reduces a level of RabGGT mRNA and/or protein and/or is an amount that reduces an activity of a RabGGT protein by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, or more, when compare to the level or activity in the absence of the agent.

In other embodiments, the invention provides methods for reducing apoptosis in a cell. The methods generally involve contacting a cell with an effective amount of an agent that increases a level and/or activity of RabGGT or a RabGGT/REP complex. The invention also provides methods of treating a disorder amenable to treatment by reducing apoptosis, the methods generally involving administering an effective amount of an agent the increases a level and/or activity or RabGGT or a RabGGT/REP complex in a cell in the individual.

An “effective amount” of an agent that increases a level and/or activity of RabGGT is an amount that increases a level of RabGGT mRNA and/or protein and/or is an amount that increases an activity of a RabGGT protein by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, or more, when compared to the level or activity in the absence of the agent.

In some embodiments, the invention provides a method of inducing apoptosis in a eukaryotic cell, wherein the method generally involves identifying a compound that is a RabGGT inhibitor; testing the ability of the compound to modulate famesyl transferase (FT) activity; modifying the compound, wherein the modified compound exhibits reduced modulation of FT activity compared to the unmodified compound, wherein inhibition of RabGGT is retained; and contacting the cell with the modified compound.

RabGGT Modulating Agents

As noted above, in some methods of the present invention, agents that reduce a level and/or activity of RabGGT are used. In other methods of the present invention, agents that increase a level and/or activity of RabGGT are used. Agents that reduce or increase a level and/or activity of RabGGT are referred to herein as “RabGGT modulators” or “RabGGT modulating agents” and include small molecule modulators, protein (or peptide) modulators, antibody modulators, and nucleic acid modulators. The RabGGT modulating agents are typically “specific” in their interaction with RabGGT, as that term is understand in the art.

Agents that reduce a level and/or activity of RabGGT include agents that reduce the protein prenyl transferase activity of RabGGT protein; agents that reduce an interaction between RabGGT and an interacting protein, where RabGGT interacting proteins include a Rab protein, an accessory protein (e.g., a REP), and a protein that binds to a Rab/RabGGT complex; agents that reduce the level of RabGGT mRNA in a cell; agents that reduce , but are not limited to, small molecule inhibitors of RabGGT enzymatic activity; antibodies specific for RabGGT; antisense RNA specific for RabGGT; interfering RNA (RNAi) specific for RabGGT; ribozymes specific for RabGGT; and the like.

In some embodiments, an agent that reduces a level and/or activity of RabGGT does not substantially reduce a level or activity of other proteins or mRNA, including famesyl transferase, e.g., the agent reduces the level or activity of another protein or mRNA by less than about 10%, less than about 5%, less than about 2%, or less than about 1%, compared to the activity or level of the protein or mRNA in the absence of the agent.

In some embodiments, agents that reduce a level and/or activity of a RabGGT/REP complex are used in a therapeutic method of the present invention. A RabGGT/REP complex includes RabGGT α and β subunits, and a Rab escort protein (REP) (e.g., REP-1, REP-2).

A RabGGT α subunit includes a protein having an amino acid sequence as set forth in SWISS-PROT Accession No. Q92696 (Genomics 38 (2), 133-140 (1996)), and homologs, analogs, and derivatives thereof, e.g., derivatives having one or more conservative amino acid substitutions. A RabGGT β subunit includes a protein having an amino acid sequence as set forth in SWISS-PROT Accession No. P53611 (Genomics 38 (2), 133-140 (1996)), and homologs, analogs, and derivatives thereof, e.g., derivatives having one or more conservative amino acid substitutions. A REP protein includes a protein having an amino acid sequence as set forth in GenBank Accession No. P24386 or P26374, and homologs, analogs, and derivatives thereof, e.g., derivatives having one or more conservative amino acid substitutions. Homologs include proteins that have from 1 to about 20 amino acid differences from a reference sequence. In general, homologs retain at least about 80%, or at least about 90% or more, of at least one activity of a protein having a reference sequence.

In some embodiments, an agent that reduces a level and/or activity of a RabGGT/REP complex does not substantially reduce a level or activity of other proteins or mRNA, including farnesyl transferase, e.g., the agent reduces the level or activity of another protein or mRNA by less than about 10%, less than about 5%, less than about 2%, or less than about 1%, compared to the activity or level of the protein or mRNA in the absence of the agent.

Biological Modulators

Modulators suitable for use herein modulate a level and/or an activity of RabGGT or a RabGGT/REP complex. A suitable modulator exhibits one or more of the following activities: 1) modulates an enzymatic activity of RabGGT or a RabGGT/REP complex; 2) modulates a level of a RabGGT protein (α and/or β subunit) or the level of a RabGGT/REP protein complex; 3) modulates the level of an mRNA that encodes a RabGGT protein (α and/or β subunit), or an mRNA that encodes a REP protein; 4) modulates the level of apoptosis in a cell; and 5) modulates a binding event between a RabGGT protein and a protein that interacts with a RabGGT protein.

Modulating Enzymatic Activity

In some embodiments, a RabGGT modulating agent modulates the protein prenyl transferase activity of RabGGT protein. In some of these embodiments, an agent increases the enzymatic activity of a RabGGT protein by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, or more, when compared to-the enzymatic activity of the RabGGT protein in the absence of the agent.

In other embodiments, an agent reduces the enzymatic activity of a RabGGT protein by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, or more, when compared to the enzymatic activity of the RabGGT protein in the absence of the agent.

In some embodiments, an agent that reduces the activity of RabGGT inhibits the activity of a RabGGT/REP complex. A suitable agent reduces the level and/or activity of a RabGGT/REP complex by at least about 10%, at least about 15%, at least about 20%, at least about 25%, at least about 30%, at least about 40%, at least about 50%, at least about 60%, at least about 70%, at least about 80%, or at least about 90% or more, compared to the level or activity of the RabGGT/REP complex in the absence of the agent.

In many embodiments, an agent that reduces RabGGT enzymatic activity has an IC₅₀ of less than 0.5 mM. Generally, a suitable agent that reduces RabGGT enzymatic activity has an IC₅₀ of from about 0.5 nM to about 500 μM, e.g., from about 0.5 nM to about 1 nM, from about 1 nM to about 5 nM, from about 5 nM to about 10 nM, from 10 nM to about 25 nM, from about 25 nM to about 50 nM, from about 50 nM to about 100 nM, from about 100 nM to about 250 nM, from about 250 nM to about 500 nM, from about 500 nM to about 1 μM, from about 1 μM to about 5 μM, from about 5 μM to about 10 μM, from about 10 μM to about 25 μM, from about 25 μM to about 50 μM, from about 50 μM to about 100 μM, from about 100 μM to about 250 μM, or from about 250 μM to about 500 μM.

Whether a given agent modulates a level and/or activity of RabGGT can be determined using any known method. For example, RabGGT enzymatic activity is quantified using a filter binding assay that measures the transfer of (³H) geranylgeranyl groups (GG) from all-trans-(³H)geranylgeranyl, pyrophosphate (³H-GGPP) to recombinant Rab3A protein (Shen and Seabra (1996) J. Biol. Chem. 271:3692; Armstrong et al. (1996) Methods in Enzymology 257:30), or as described in the Examples.

Protein Level

In some embodiments, an agent modulates a level of RabGGT protein in a cell. In some of the embodiments, an agent increases the level of a RabGGT protein in a cell by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, or more, when compared to the level in a control cell in the absence of the agent.

In other embodiments, an agent decreases the level of a RabGGT protein in a cell by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, or more, when compared to the level in a control cell in the absence of the agent.

The level of RabGGT protein in a cell can be determined using a standard, well-known immunological assay, e.g., an enzyme-linked immunosorbent assay, a protein blot assay, a radioimmunoassay, and the like, using antibody specific for RabGGT, which antibody is directly or indirectly labeled.

Direct and indirect antibody labels are known in the art. An antibody may be labeled with a radioisotope, an enzyme, a fluorescer (e.g., a fluorescent protein or a fluorescent dye), a chemiluminescer, or other label for direct detection. Alternatively, a second stage antibody or reagent is used to amplify the signal. Such reagents are well known in the art. For example, the primary antibody may be conjugated to biotin, with horseradish peroxidase-conjugated avidin added as a second stage reagent. Final detection uses a substrate that undergoes a color change in the presence of the peroxidase. Alternatively, the secondary antibody conjugated to a fluorescent compound, e.g. fluorescein, rhodamine, Texas red, etc. The absence or presence of antibody binding may be determined by various methods, including flow cytometry of dissociated cells, microscopy, radiography, scintillation counting, etc.

Fluorescent proteins include, but are not limited to, a green fluorescent protein (GFP), e.g., a GFP derived from Aequoria victoria or a derivative thereof; a GFP from another species such as Renilla reniformis, Renilla mulleri, or Ptilosarcus guernyi, as described in, e.g., WO 99/49019 and Peelle et al. (2001) J. Protein Chem. 20:507-519; any of a variety of fluorescent and colored proteins from Anthozoan species, as described in, e.g., Matz et al. (1999) Nature Biotechnol. 17:969-973; and the like.

Enzyme labels include, but are not limited to, luciferase, β-galactosidase, horse radish peroxidase, and the like. Where the label is an enzyme that yields a detectable product, the product can be detected using an appropriate means, e.g., β-galactosidase can, depending on the substrate, yield colored product, which is detected spectrophotometrically, or a fluorescent product; luciferase can yield a luminescent product detectable with a luminometer; etc.

RabGGT mRNA Level

In some embodiments, an agent modulates the level of a RabGGT mRNA in a cell, e.g., the agent modulates the level of mRNA that comprises a nucleotide sequence that encodes a RabGGT protein. Agents that modulate the level of a RabGGT mRNA include agents that modulate the rate of transcription of the mRNA, agents that modulate binding of a transcription factor(s) or other regulatory protein(s) to a RabGGT gene regulatory element (e.g., enhancer, promoter, and the like); agents that modulate the stability of RabGGT mRNA stability; and the like.

In some embodiments, an agent increases the level of RabGGT mRNA by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, or more, when compared to the level in the absence of the agent.

In other embodiments, an agent decreases the level of RabGGT mRNA by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, or more, when compared to the level in the absence of the agent.

The level of RabGGT mRNA in a cell is readily determined using any known method. In general, nucleic acids that hybridize specifically to a RabGGT mRNA are used. A number of methods are available for analyzing nucleic acids for the presence and/or level of a specific mRNA in a cell or in a sample. The mRNA may be assayed directly or reverse transcribed into cDNA for analysis. Suitable methods include, but are not limited to, in situ nucleic acid hybridization methods, quantitative RT-PCR, nucleic acid blotting methods, and the like.

The nucleic acid may be amplified by conventional techniques, such as the polymerase chain reaction (PCR), to provide sufficient amounts for analysis. The mRNA may be reverse transcribed, then subjected to PCR (rtPCR). The use of the polymerase chain reaction is described in Saiki, et al. (1985), Science 239:487, and a review of techniques may be found in Sambrook, et al. Molecular Cloning: A Laboratory Manual, CSH Press 1989, pp. 14.2-14.33.

A detectable label may be included in an amplification reaction. Suitable labels include fluorochromes, e.g. fluorescein isothiocyanate (FITC), rhodamine, Texas Red, phycoerythrin, allophycocyanin, 6-carboxyfluorescein (6-FAM), 2′, 7′-dimethoxy-4′, 5′-dichloro-6-carboxyfluorescein (JOE), 6-carboxy-X-rhodamine (ROX), 6-carboxy-2′, 4′, 7′, 4,7-hexachlorofluorescein (HEX), 5-carboxyfluorescein (5-FAM) or N,N,N′,N′-tetramethyl-6-carboxyrhodamine (TAMRA), radioactive labels, e.g. ³²P, ³⁵S, ³H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

A variety of different methods for determining the nucleic acid abundance in a sample are known to those of skill in the art, where particular methods of interest include those described in: Pietu et al., Genome Res. (June 1996) 6: 492-503; Zhao et al., Gene (Apr. 24, 1995) 156: 207-213; Soares, Curr. Opin. Biotechnol. (October 1997) 8: 542-546; Raval, J. Pharmacol Toxicol Methods (November 1994) 32: 125-127; Chalifour et al., Anal. Biochem (Feb. 1, 1994) 216: 299-304; Stolz & Tuan, Mol. Biotechnol. (December 19960 6: 225-230; Hong et al., Bioscience Reports (1982) 2: 907; and McGraw, Anal. Biochem. (1984) 143: 298. Also of interest are the methods disclosed in WO 97/27317, the disclosure of which is herein incorporated by reference.

In some embodiments, RabGGT mRNA levels are quantitated using quantitative rtPCR. Methods of quantitating a given message using rtPCR are known in the art. In some of these embodiments, dye-labeled primers are used. In other embodiments, a double-stranded DNA-binding dye, such as SYBR®, is used, as described in the Examples. Quantitative fluorogenic RT-PCR assays are well known in the art, and can be used in the present methods to detect a level of RabGGT mRNA. See, e.g., Pinzani et al. (2001) Regul. Pept. 99:79-86; and Yin et al. (2001) Immunol. Cell Biol. 79:213-221.

Apoptosis

In some embodiments, an agent that modulates a level and/or activity of RabGGT mRNA and/or protein induces apoptosis in a eukaryotic cell.

Whether a given agent inhibits RabGGT and induces apoptosis in a eukaryotic cell can be determined using any known method. Assays can be conducted on cell populations or an individual cell, and include morphological assays and biochemical assays. A-non-limiting example of a method of determining the level of apoptosis in a cell population is TUNEL (TdT-mediated dUTP nick-end labeling) labeling of the 3′-OH free end of DNA fragments produced during apoptosis (Gavrieli et al. (1992) J. Cell Biol. 119:493). The TUNEL method consists of catalytically adding a nucleotide, which has been conjugated to a chromogen system or a to a fluorescent tag, to the 3′-OH end of the 180-bp (base pair) oligomer DNA fragments in order to detect the fragments. The presence of a DNA ladder of 180-bp oligomers is indicative of apoptosis. Procedures to detect cell death based on the TUNEL method are available commercially, e.g., from Boehringer Mannheim (Cell Death Kit) and Oncor (Apoptag Plus). Another marker that is currently available is annexin, sold under the trademark APOPTEST™. This marker is used in the “Apoptosis Detection Kit,” which is also commercially available, e.g., from R&D Systems. During apoptosis, a cell membrane's phospholipid asymmetry changes such that the phospholipids are exposed on the outer membrane. Annexins are a homologous group of proteins that bind phospholipids in the presence of calcium. A second reagent, propidium iodide (PI), is a DNA binding fluorochrome. When a cell population is exposed to both reagents, apoptotic cells stain positive for annexin and negative for PI, necrotic cells stain positive for both, live cells stain negative for both. Other methods of testing for apoptosis are known in the art and can be used, including, e.g., the method disclosed in U.S. Pat. No. 6,048,703.

Modulating a Binding Event

In some embodiments, an agent that modulates a RabGGT activity modulates a binding event between RabGGT and a RabGGT interacting protein. RabGGT interacting proteins include, but are not limited to, a Rab protein; a Rab escort protein (REP); and a protein that binds to a Rab/RabGGT complex.

In some embodiments, an agent increases binding between RabGGT and a RabGGT interacting protein by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, or more, when compared to the binding in the absence of the agent.

In some embodiments, an agent reduces binding between RabGGT and a RabGGT interacting protein by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, or more, when compared to the binding in the absence of the agent.

In some embodiments, the agent reduces binding between RabGGT and a Rab protein.

Rab proteins are known in the art. For example, at least 30 human Rab proteins are known, and include Rab1a, Rab1b, Rab2a, Rab2b, Rab3a, Rab3b, Rab3c, Rab3d, Rab4a, Rab4b, Rab5a, Rab5b, Rab5c, Rab6a, Rab6b, Rab6c, Rab7, Rab8a, Rab8b, Rab9a, Rab9b, Rab10, Rab11a, Rab11b, Rab12, Rab13, Rab14, Rab15, Rab17, Rab18, Rab19, Rab20, Rab21, Rab22a, Rab22b, Rab22c, Rab23, Rab24, Rab25, Rab26, Rab27a, Rab27b, Rab28, Rab29, Rab30, Rab32, Rab33a, Rab33b, Rab34, Rab35, Rab36, Rab37, Rab38, Rab39a, Rab39b. See e.g., Seabra et al. (2002) Trends Mol. Med. 8:23-30.

In some embodiments, an agent inhibits binding between a Rab protein and REP protein. RabGGT prenylates Rab only when Rab is in a complex with REP. Therefore, an agent that reduces a Rab/REP interaction also reduces Rab/RabGGT binding. Accordingly, agents that reduce Rab/REP binding are suitable for use in a subject methods. Rab/REP interaction via a RabF motif is a target for inhibiting Rab/REP binding. The RabF motif has been described in the art. See, e.g., Pereira-Leal et al. (2003) Biochem. Biophys. Res. Comm. 301:92-97. An agent that inhibits binding of a REP protein to a RabF motif is suitable for use in a subject method. Human REP proteins are known in the art, and the amino acid sequences have been reported. See, e.g., GenBank Accession No. NP_{—}000381 or P24386 for human REP-1; NP_{—}001812 for human REP-2; etc.

Whether an agent modulates binding between two proteins, e.g., between a Rab protein and a RabGGT protein, between a Rab protein and a REP protein, between a Rab/REP complex and RabGGT, can be determined using standard methods that are well known in the art. Suitable methods include, but are not limited to, a yeast two-hybrid assay; a fluorescence resonance energy transfer (FRET) assay; a bioluminescence resonance energy transfer (BRET) assay; a fluorescence quenching assay; a fluorescence anisotropy assay; an immunological assay; and an assay involving binding of a detectably labeled protein to an immobilized protein.

FRET involves the transfer of energy from a donor fluorophore in an excited state to a nearby acceptor fluorophore. For this transfer to take place, the donor and acceptor molecules must in close proximity (e.g., less than 10 nanometers apart, usually between 10 and 100 Å apart), and the emission spectra of the donor fluorophore must overlap the excitation spectra of the acceptor fluorophore. In one non-limiting example, a fluorescently labeled RabGGT protein serves as a donor and/or acceptor in combination with a second fluorescent protein (e.g., a Rab protein) or dye; e.g., a fluorescent protein as described in Matz et al. (1999) Nature Biotechnology 17:969-973; a green fluorescent protein (GFP); a GFP from Aequoria victoria or fluorescent mutant thereof, e.g., as described in U.S. Pat. Nos. 6,066,476; 6,020,192; 5,985,577; 5,976,796; 5,968,750; 5,968,738; 5,958,713; 5,919,445; 5,874,304, the disclosures of which are herein incorporated by reference; a GFP from another species such as Renilla reniformis, Renilla mulleri, or Ptilosarcus guernyi, as described in, e.g., WO 99/49019 and Peelle et al. (2001) J. Protein Chem. 20:507-519; “humanized” recombinant GFP (hrGFP) (Stratagene); other fluorescent dyes, e.g., coumarin and its derivatives, e.g. 7-amino-4-methylcoumarin, aminocoumarin, bodipy dyes, such as Bodipy FL, cascade blue, fluorescein and its derivatives, e.g. fluorescein isothiocyanate, Oregon green, rhodamine dyes, e.g. texas red, tetramethylrhodamine, eosins and erythrosins, cyanine dyes, e.g. Cy3 and Cy5, macrocyclic chelates of lanthanide ions, e.g. quantum dye, etc., chemilumescent dyes, e.g., luciferases.

BRET is a protein-protein interaction assay based on energy transfer from a bioluminescent donor to a fluorescent acceptor protein. The BRET signal is measured by the amount of light emitted by the acceptor to the amount of light emitted by the donor. The ratio of these two values increases as the two proteins are brought into proximity. The BRET assay has been amply described in the literature. See, e.g., U.S. Pat. Nos. 6,020,192; 5,968,750; and 5,874,304; and Xu et al. (1999) Proc. Natl. Acad. Sci. USA 96:151-156. BRET assays may be performed by analyzing transfer between a bioluminescent donor protein and a fluorescent acceptor protein. Interaction between the donor and acceptor proteins can be monitored by a change in the ratio of light emitted by the bioluminescent and fluorescent proteins. In one non-limiting example, a RabGGT protein serves as donor and/or acceptor protein.

Fluorescent RabGGT can be produced by generating a construct encoding a protein comprising a RabGGT protein and a fluorescent fusion partner, e.g., a fluorescent protein as described in Matz et al. ((1999) Nature Biotechnology 17:969-973), a green fluorescent protein from any species or a derivative thereof; e.g., a GFP from another species such as Renilla reniformis, Renilla mulleri, or Ptilosarcus guernyi, as described in, e.g., WO 99/49019 and Peelle et al. (2001) J. Protein Chem. 20:507-519; a GFP from Aequoria victoria or fluorescent mutant thereof, e.g., as described in U.S. Pat. Nos. 6,066,476; 6,020,192; 5,985,577; 5,976,796; 5,968,750; 5,968,738; 5,958,713; 5,919,445; 5,874,304. Generation of such a construct, and production of a RabGGT/fluorescent protein fusion protein is well within the skill level of those of ordinary skill in the art.

Alternatively, binding may be assayed by fluorescence anisotropy. Fluorescence anisotropy assays are amply described in the literature. See, e.g., Jameson and Sawyer (1995) Methods Enzymol. 246:283-300.

In some embodiments, the method of determining whether an agent modulates a protein/protein interaction is a yeast two-hybrid assay system or a variation thereof The yeast two-hybrid screen has been described in the literature. See, e.g., Zhu and Kahn (1997) Proc. Natl. Acad. Sci. U.S.A. 94:13063-13068; Fields and Song (1989) Nature 340:245-246; and U.S. Pat. No. 5,283,173; Chien et al. (1991) Proc. Natl. Acad. Sci. U.S.A. 88:9578-9581.

Protein/protein binding can also be assayed by other methods well known in the art, for example, immunoprecipitation with an antibody that binds to the protein in a complex, followed by analysis by size fractionation of the immunoprecipitated proteins (e.g. by denaturing or nondenaturing polyacrylamide gel electrophoresis); Western analysis; non-denaturing gel electrophoresis, etc.

Chemical Features of Modulators

In some embodiments, an agent that modulates a level and/or an activity of a RabGGT protein and/or a RabGGT/REP complex is a compound that binds to the binding pocket for the substrate prenyl moiety and/or the peptide substrate in the RabGGT active site. A suitable compound comprises moieties that provide for interactions with amino acid side chains that normally interact with substrate prenyl moiety and/or peptide substrate in the RabGGT active site. Features that a suitable compound possesses include one or more of: (1) zinc binding; (2) hydrogen bonding to specific amino acid side chains; (3) a hydrophobic moiety; (4) a size sufficient to occlude the binding site for the prenyl and/or the peptide substrate; and/or a size sufficient to interface with the size limitations embodied by the binding pocket of the RabGGT alpha and beta subunits, and defined by their respective structure coordinates.

In some embodiments, a suitable modulator of enzymatic activity of RabGGT or a RabGGT/REP complex is a benzodiazepine. In other embodiments, a suitable modulator of enzymatic activity of RabGGT or a RabGGT/REP complex is a tetrahydroquinoline.

In other embodiments, a suitable modulator of enzymatic activity of RabGGT or a RabGGT/REP complex may comprise one or more of the side chains, moieties, or groups, or any combinations thereof, of the compounds disclosed in U.S. Pat. No. 6,011,029; U.S. Pat. No. 6,387,926; and/or U.S. Pat. No. 6,458,783, which are hereby incorporated by reference herein in their entirety.

In one embodiment, a suitable modulator of RabGGT or a RabGGT/REP complex may comprise a side chain, moiety, or group capable of chelating zinc, and/or coordinating with zinc. Examples of zinc chelators and/or cooridinators include, but are not limited to the following: thiol, cysteine, cysteine derivative, hydroxamic acid, hydroxamic acid derivative, barbituric acid, barbituric acid derivative, pyridyl, imidazolyl, methionine, nitrogen-containing heterocycles, or other groups known in the art that are capable of chelating and/or coordinating with zinc, or disclosed or referenced herein.

In another embodiment, a suitable modulator of RabGGT or a RabGGT/REP complex may comprise a hydrophobic or aromatic side chain, moiety, or group. Examples of such groups include, but are not limited to the following: phenyl, planar phenyl, aryl, substituted phenyl, cyano substituted phenyl, a cyanobenzene, substituted aryl, heteroaryl, substituted heteroaryl, or other hydrophobic or aromatic side chain, moiety, or group known in the art, or disclosed or referenced herein.

In another embodiment, a suitable modulator of RabGGT or a RabGGT/REP complex may comprise one, two, three, four, or more hydrophobic or aromatic side chains, moieties, or groups.

In another embodiment, a suitable modulator of RabGGT or a RabGGT/REP complex may comprise a side chain, moiety, or group capable of ligating with a water molecule and/or forming one or more hydrogen bonds with a water molecule.

In yet another embodiment, a suitable modulator of RabGGT or a RabGGT/REP complex may comprise a large multicyclic aromatic and/or hydrophobic side chain, moiety, or group. In yet another embodiment, a suitable modulator of RabGGT or a RabGGT/REP complex may not comprise a large multicyclic aromatic and/or hydrophobic side chain, moiety, or group. Examples of such multicyclic aromatic and/or hydrophobic side chains, moieties, or groups may be found in the teachings of I. M. Bell et al, J. Med. Chem. 45:2388 (2002), which is hereby incorporated herein by reference in its entirety.

A suitable modulator of RabGGT or a RabGGT/REP complex may comprise any combination of one, two, three, four, five, six, seven, eight, nine, ten, or more of the above specified characteristics.

Pharmacophores

Suitable modulators of RabGGT or RabGGT/REP activity are pharmacophores that possess appropriate size, volume, charge, and hydrophobicity features to allow interactions with amino acid side chains in the active site that normally interact with prenyl and/or peptide substrates. Such features may be used to identify compounds that are modulators of RabGGT or RabGGT/REP complex activity.

Features can include topological indices, physicochemical properties, electrostatic field parameters, volume and surface parameters, etc. Other features include, but are not limited to, molecular volume and surface areas, dipole moments, octanol-water partition coefficients, molar refractivities, heats of formation, total energies, ionization potentials, molecular connectivity indices, substructure keys. Such descriptors and their use in the fields of Quantitative Structure-Activity Relationships (QSAR) and molecular diversity are reviewed in Kier, L. B. and Hall L. H., Molecular Connectivity in Chemistry and Drug Research, Academic Press, New York (1976); Kier, L. B. and Hall L. H., Molecular Connectivity in Structure-Activity Analysis, Research Studies Press, Wiley, Letchworth (1986); Kubinyi, H., Methods and Principles in Medicinal Chemistry, Vol. 1, VCH, Weinheim (1993); and P. V. R. Scheyler, Encyclopedia of Computational Chemistry, Wiley (1998).

In some embodiments, a modulator of an activity of RabGGT or a RabGGT/REP complex is identified by computational quantitative structure activity relationship (QSAR) modeling techniques as a screening device for potency as an inhibitor or activator. Structure-activity relationship (SAR) analysis is performed using any known method. See, e.g., U.S. Pat. No. 6,344,334; U.S. Pat. No. 6,208,942; U.S. Pat. No. 6,453,246; U.S. Pat. No. 6,421,612.

Suitable compounds can be identified using a selection approach that involves (1) identifying a set of compounds for analysis; (2) collecting, acquiring or synthesizing the identified compounds; (3) analyzing the compounds to determine one or more physical, chemical and/or bioactive properties (structure-property data); and (4) using the structure-property data to identify another set of compounds for analysis in the next iteration. These steps can be repeated multiple times, as necessary to derive suitable compounds with desired properties.

Suitable compounds may also be identified by subjecting putative modulators of the RabGGTase protein to virtual screens that predict the overall fit of the modulator to the putative binding site(s) of the RabGGTase protein, its alpha subunit, its beta subunit, the RabGGTase/Rep complex, and/or the RabGGTase/Rep/substrate ternary complex. The DOCK3.5 algorithm, among others described herein, may be used for virtually screening RabGGTase modulators. DOCK3.5 is an automatic algorithm to screen small-molecule databases for ligands that could bind to a given receptor (Meng, E. C., et al., 1992, J. Comp. Chem. 15:505). DOCK3.5 characterizes the surface of the active site to be filled with sets of overlapping spheres. The generated sphere centers constitute an irregular grid that is matched to the atomic centers of the potential ligands. The quality of the fit of the ligand to the site is judged by either the shape complementarity or by a simplified estimated interaction energy. Putative RabGGTase modulators having the best shape complementarity scores and the best force field scores may be selected from the screen. The resulting virtual modulators may then be visually screened independently in the context of the RabGGTase binding pocket described herein using the molecular display software Insight II (Biosym Inc., San Diego, Calif.). Such compounds can then be confirmed to have RabGGTase modulating activity by subjecting these compounds to screening assays described herein.

Preferred RabGGTase modulators have a complementarity score of at least about 10, 20, 30, 40, 50, 60, 70, 80, 90, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, or greater. In this context, “about” should be construed to represent 1 to 13 more or less than the stated complementarity score.

Small Molecule Modulators

In some embodiments, an agent that increases or reduces a level and/or an activity of RabGGT or a RabGGT/REP complex is a small molecule. Small molecule agents are generally small organic or inorganic compounds having a molecular weight of more than 50 and less than about 2,500 daltons. Specifically, small molecule agents may be at least about 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, or 2500. In this context, “about” should be construed to represent more or less than 1 to 25 daltons than the indicated amount.

Suitable agents may comprise functional groups necessary for structural interaction with proteins, particularly hydrogen bonding, and may include at least an amine, carbonyl, hydroxyl or carboxyl group, and may contain at least two of the functional chemical groups. The agents may comprise cyclical carbon or heterocyclic structures and/or aromatic or polyaromatic structures substituted with one or more of the above functional groups. Suitable active agents are also found among biomolecules including peptides, saccharides, fatty acids, steroids, purines, pyrimidines, derivatives, structural analogs or combinations thereof.

In some embodiments, agents that reduce enzymatic activity of RabGGT or level of enzymatically active RabGGT are of the following formula:

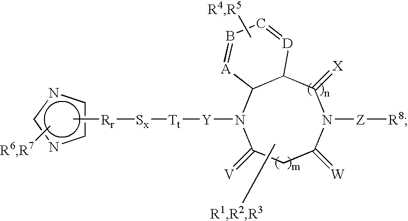

or an enantiomer, diastereomer, pharmaceutically acceptable salt, prodrug, or solvate thereof, where m, n, r, s, and 1 are 0 or 1;

p is 0, 1, or 2;

V, W, and X are selected from oxygen, hydrogen, R¹, R², or R³;

Z and Y are selected from CHR⁹, SO₂, SO₃, CO, CO₂, O, NR¹⁰, SO₂NR¹¹, CONR¹²,

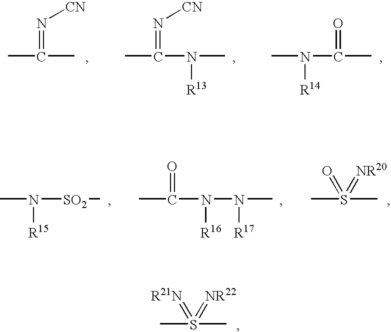

or Z may be absent;

R⁶, R⁷, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹, R³⁰, R³¹, R³² R³³, R³⁴, R³⁵, R³⁶, R³⁷, and R³⁸, are each independently selected from hydrogen, lower alkyl, substituted alkyl, aryl, or substituted aryl;

R⁴ and R⁵ are independently selected from hydrogen, halo, nitro, cyano, and U-R²³;

U is selected from sulfur, oxygen, NR²⁴, CO, SO, SO₂, CO₂, NR²⁵CO₂, NR²⁶CONR²⁷; NR²⁸SO₂, NR²⁹SO₂NR³⁰, SO₂NR³¹, NR³²CO, CONR³³, PO₂R³⁴, and PO₃R³⁵ or U is absent;

R¹, R², and R³ are each independently selected from hydrogen, alkyl, alkoxycarbonyl, substituted alkyl, alkenyl, substituted alkenyl, alkynyl, substituted alkynyl, arakyl, cycolalkyl, aryl, substituted aryl, heterocyclo, substituted heterocyclo, cyano, carboxyl, carbamyl (e.g., CONH₂) or substituted carbamyl further selected from CONH alkyl, CONH aryl, CONH aralkyl or cases where there are two substituents on the nitrogen selected from alkyl, aryl, or aralkyl, ; R⁸ and R²³ are independently selected from hydrogen, alkyl, substituted alkyl, alkenyl, substituted alkenyl, aalkynyl, substituted alkynyl, aralkyl, cycloalkyl, aryl, substituted aryl, heterocyclo, substituted heterocyclo;

any two of R¹, r², and R³ can be joined to form a cycloalkyl group;

R, S, and T are selected from CH₂, CO, and CH(CH₂)pQ, wherein Q is NR³⁶R³⁷, OR³⁸, or CN; and

A, B, and D are carbon, oxygen, sulfur or nitrogen, with the proviso that

1) when m is zero, then V and W are not both oxygen; or

2) W and X together can be oxygen only if Z is either absent, O, NR¹⁰, CHR⁹,

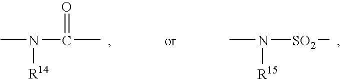

3) R²³ may be hydrogen except with U is SO₂, CO₂, or

4) R⁸ may be hydrogen except when Z is SO₂, CO₂ or

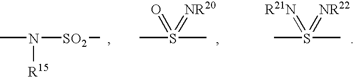

In other embodiments, agents that reduce enzymatic activity of RabGGT or level of enzymatically active RabGGT are of the following formula:

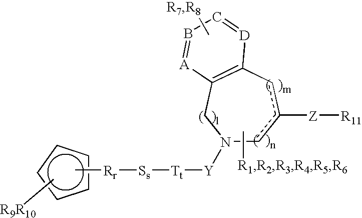

or an enantiomer, diastereomer, pharmaceutically acceptable salt, prodrug, or solvate thereof,

l, m, r, s, and t are 0 or 1;

N is 0, 1, or 2;

Y is selected from CHR¹², SO₂, SO₃, CO, CO₂, Y is selected from the group consisting of CHR¹² SO₂, SO₃, CO, CO₂, O, NR¹³, SO₂NR¹⁴, CONR¹⁵, C(NCN), C(NCN)NR¹⁶, NR¹⁷CO, NR¹⁸SO₂, CONR¹⁹NR²⁰, SO₂NR21NR22, S(O)(NR²³), S(NR²⁴)NR²⁵), or without Y;

Z is selected from the group consisting of CR¹²,S, SO, SO₂,SO₃CO,CO₂, O,NR¹³SO₂NR¹⁴,CONR¹⁵,NR²⁶NR²⁷,ONR²⁸,NR²⁹O,NR³⁰SO₂NR³¹,NR³²SO,NR³³C(NCN), NR³⁴,C(NCN)NR³⁵, NR³⁶CO, NR³⁷CO, NR³⁷CONR³⁸, NR³⁹CO₂, OCONR⁴⁰, S(O)(NR⁴¹), S(NR⁴²)(NR⁴³) or CHR¹²;

or without Z;

R⁷, R⁸ are selected from the group consisting of hydrogen, halo, nitro, cyano and U—R⁴⁴;

U is selected from the group consisting of S, O, NR⁴⁵, CO, SO, SO₂, CO₂, NR⁴⁶CO₂, NR⁴⁷CONR⁴⁸, NR⁴⁹SO₂, NR⁵⁰SO₂NR⁵¹, SO₂NR⁵², NR⁵³CO, CONR⁵⁴, PO₂R⁵⁵ and PO₂R⁵⁶ or without U;

R⁹, R¹⁰, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁵⁷, R⁵⁸ and R⁵⁹ are selected from the group consisting of hydrogen, lower alkyl, aryl, heterocyclo, substituted alkyl or aryl or substituted heterocyclo;

R¹¹ and R⁴⁴ are selected from the group consisting of hydrogen, alkyl, substituted alkyl, alkenyl, substituted alkenyl, alkynyl, sub alkynyl, aralkyl, cycloalkyl, aryl, substituted aryl, heterocyclo, substituted heterocyclo;

R¹, R², R³, R⁴, R⁵, and R⁶ are selected from the group consisting of hydrogen, alkyl, substituted alkyl, alkenyl, substituted alkenyl, alkynyl, cycloalkyl, aryl, substituted aryl, heterocyclo, substituted heterocyclo, cyano, carboxy, carbamyl (e.g. CONH₂) substituted carbamyl (where nitrogen may be substituted by groups selected from hydrogen, alkyl, substituted alkyl, aryl or aralkyl, substituted aryl, heterocyclo, sub-situated heterocyclo) alkoxycarbonyl; any two of R¹, R², R³, R⁴, R⁵, and R⁶ can join to form a cycloalkyl group; any two of R¹, R², R³, R⁴, R⁵, and R⁶ together can by oxo, except when the carbon atom bearing the substituent is part of a double bond;

R, S, T are selected from the group consisting of CH₂, CO and CH(CH₂)Q wherein Q is NR⁵⁷R⁵⁸, OR⁵⁹, or CN; and p is 0, 1 or 2;

A, B, C are carbon, oxygen, sulfur or nitrogen; D is carbon, oxygen, sulfur or nitrogen or without D,

with the provisos that:

1. When l and m are both 0, n is not 0;

2. R¹¹ may be hydrogen except when Z is SO, or when Z is O, NR¹³ or S and the carbon to which it is attached is part of a double bond or when Y is SO₂, CO₂, NR¹⁸SO₂, S(O)(NR²³), or S(NR²⁴)(NR²⁵); and

3. R⁴⁴ may be hydrogen except when U is SO, SO₂, NR⁴⁶CO₂ or NR⁴⁹SO₂.

In some embodiments, the agents disclosed in U.S. Pat. No. 6,011,029; U.S. Pat. No. 6,387,926; and/or U.S. Pat. No. 6,458,783 are specifically excluded from the present invention.

Protein Modulators

Agents that modulate an activity of a RabGGT include protein modulators. In some embodiments, an active agent is a peptide. Suitable peptides include peptides of from about 3 amino acids to about 50, from about 5 to about 30, or from about 10 to about 25 amino acids in length. In some embodiments, a peptide exhibits one or more of the following activities: inhibits binding of RabGGT to a RabGGT interacting protein; inhibits interaction between an α and a β subunit of RabGGT; inhibits an enzymatic activity of RabGGT. Peptides can include naturally-occurring and non-naturally occurring amino acids. Peptides may comprise D-amino acids, a combination of D- and L-amino acids, and various “designer” amino acids (e.g., β-methyl amino acids, Cα-methyl amino acids, and Nα-methyl amino acids, etc.) to convey special properties to peptides. Additionally, peptide may be a cyclic peptide. Peptides may include non-classical amino acids in order to introduce particular conformational motifs. Any known non-classical amino acid can be used. Non-classical amino acids include, but are not limited to, 1,2,3,4-tetrahydroisoquinoline-3-carboxylate; (2S,3S)-methylphenylalanine, (2S,3R)-methyl-phenylalanine, (2R,3S)-methyl-phenylalanine and (2R,3R)-methyl-phenylalanine; 2-aminotetrahydronaphthalene-2-carboxylic acid; hydroxy-1,2,3,4-tetrahydroisoquinoline-3-carboxylate; β-carboline (D and L); HIC (histidine isoquinoline carboxylic acid); and HIC (histidine cyclic urea). Amino acid analogs and peptidomimetics may be incorporated into a peptide to induce or favor specific secondary structures, including, but not limited to, LL-Acp (LL-3-amino-2-propenidone-6-carboxylic acid), a β-turn inducing dipeptide analog; β-sheet inducing analogs; β-turn inducing analogs; α-helix inducing analogs; γ-turn inducing analogs; Gly-Ala turn analog; amide bond isostere; tretrazol; and the like.

A peptide may be a depsipeptide, which may be a linear or a cyclic depsipeptide. Kuisle et al. (1999) Tet. Letters 40:1203-1206. “Depsipeptides” are compounds containing a sequence of at least two alpha-amino acids and at least one alpha-hydroxy carboxylic acid, which are bound through at least one normal peptide link and ester links, derived from the hydroxy carboxylic acids, where “linear depsipeptides” may comprise rings formed through S—S bridges, or through an hydroxy or a mercapto group of an hydroxy-, or mercapto-amino acid and the carboxyl group of another amino- or hydroxy-acid but do not comprise rings formed only through peptide or ester links derived from hydroxy carboxylic acids. “Cyclic depsipeptides” are peptides containing at least one ring formed only through peptide or ester links, derived from hydroxy carboxylic acids.

Peptides may be cyclic or bicyclic. For example, the C-terminal carboxyl group or a C-terminal ester can be induced to cyclize by internal displacement of the —OH or the ester (—OR) of the carboxyl group or ester respectively with the N-terminal amino group to form a cyclic peptide. For example, after synthesis and cleavage to give the peptide acid, the free acid is converted to an activated ester by an appropriate carboxyl group activator such as dicyclohexylcarbodiimide (DCC) in solution, for example, in methylene chloride (CH₂Cl₂), dimethyl formamide (DMF) mixtures. The cyclic peptide is then formed by internal displacement of the activated ester with the N-terminal amine. Internal cyclization as opposed to polymerization can be enhanced by use of very dilute solutions. Methods for making cyclic peptides are well known in the art

The term “bicyclic” refers to a peptide in which there exists two ring closures. The ring closures are formed by covalent linkages between amino acids in the peptide. A covalent linkage between two nonadjacent amino acids constitutes a ring closure, as does a second covalent linkage between a pair of adjacent amino acids which are already linked by a covalent peptide linkage. The covalent linkages forming the ring closures may be amide linkages, i.e., the linkage formed between a free amino on one amino acid and a free carboxyl of a second amino acid, or linkages formed between the side chains or “R” groups of amino acids in the peptides. Thus, bicyclic peptides may be “true” bicyclic peptides, i.e., peptides cyclized by the formation of a peptide bond between the N-terminus and the C-terminus of the peptide, or they may be “depsi-bicyclic” peptides, i.e., peptides in which the terminal amino acids are covalently linked through their side chain moieties.

A desamino or descarboxy residue can be incorporated at the terminii of the peptide, so that there is no terminal amino or carboxyl group, to decrease susceptibility to proteases or to restrict the conformation of the peptide. C-terminal functional groups include amide, amide lower alkyl, amide di(lower alkyl), lower alkoxy, hydroxy, and carboxy, and the lower ester derivatives thereof, and the pharmaceutically acceptable salts thereof.

In addition to the foregoing N-terminal and C-terminal modifications, a peptide or peptidomimetic can be modified with or covalently coupled to one or more of a variety of hydrophilic polymers to increase solubility and circulation half-life of the peptide. Suitable nonproteinaceous hydrophilic polymers for coupling to a peptide include, but are not limited to, polyalkylethers as exemplified by polyethylene glycol and polypropylene glycol, polylactic acid, polyglycolic acid, polyoxyalkenes, polyvinylalcohol, polyvinylpyrrolidone, cellulose and cellulose derivatives, dextran and dextran derivatives, etc. Generally, such hydrophilic polymers have an average molecular weight ranging from about 500 to about 100,000 daltons, from about 2,000 to about 40,000 daltons, or from about 5,000 to about 20,000 daltons. The peptide can be derivatized with or coupled to such polymers using any of the methods set forth in Zallipsky, S., Bioconjugate Chem., 6:150-165 (1995); Monfardini, C, et al., Bioconjugate Chem., 6:62-69 (1995); U.S. Pat. Nos. 4,640,835; 4,496,689; 4,301,144; 4,670,417; 4,791,192; 4,179,337 or WO 95/34326.

Another suitable agent for modulating an activity of RabGGT is a peptide aptamer. Peptide aptamers are peptides or small polypeptides that act as dominant inhibitors of protein function. Peptide aptamers specifically bind to target proteins, blocking their function ability. Kolonin and Finley, PNAS (1998) 95:14266-14271. Due to the highly selective nature of peptide aptamers, they may be used not only to target a specific protein, but also to target specific functions of a given protein (e.g a signaling function). Further, peptide aptamers may be expressed in a controlled fashion by use of promoters which regulate expression in a temporal, spatial or inducible manner. Peptide aptamers act dominantly; therefore, they can be used to analyze proteins for which loss-of-function mutants are not available.

Peptide aptamers that bind with high affinity and specificity to a target protein may be isolated by a variety of techniques known in the art. Peptide aptamers can be isolated from random peptide libraries by yeast two-hybrid screens (Xu et al., PNAS (1997) 94:12473-12478). They can also be isolated from phage libraries (Hoogenboom et al., Immunotechnology (1998) 4:1-20) or chemically generated peptides/libraries.

Antibody Modulators

In some embodiments, an agent that increases or reduces a level and/or activity of RabGGT is an antibody specific for RabGGT. Antibodies include naturally-occurring antibodies, artificial antibodies, intrabodies, antibody fragments, and the like, that specifically bind a RabGGT polypeptide. In some embodiments, a subject antibody binds specifically to native RabGGT protein, e.g., to native RabGGT protein present in vivo in an individual.

In many embodiments, a subject antibody is isolated, e.g., is in an environment other than its naturally-occurring environment. In some embodiments, a subject antibody is synthetic. Suitable antibodies are obtained by immunizing a host animal with peptides comprising all or a portion of the subject protein. Suitable host animals include mouse, rat, sheep, goat, hamster, rabbit, etc. The host animal is any mammal that is capable of mounting an immune response to a RabGGT protein, where representative host animals include, but are not limited to, e.g., rabbits, goats, mice, etc.

The immunogen may comprise the complete protein, or fragments and derivatives thereof. Preferred immunogens comprise all or a part of the protein. Immunogens are produced in a variety of ways known in the art, e.g., expression of cloned genes using conventional recombinant methods, followed by in vitro production of the RabGGT polypeptide; isolation of a RabGGT polypeptide; preparation of fragments of a RabGGT polypeptide using well-known methods, etc.

In some embodiments, a subject antibody is bound to a solid support or an insoluble support. Insoluble supports include, but are not limited to, beads (including plastic beads, magnetic beads, and the like); plastic plates (e.g., microtiter plates); membranes (e.g., polyvinyl pyrrolidone, nitrocellulose, and the like); and the like.

For preparation of polyclonal antibodies, the first step is immunization of the host animal with the target protein, where the target protein will preferably be in substantially pure form, comprising less than about 1% contaminant. The immunogen may comprise the complete target protein, fragments or derivatives thereof. To increase the immune response of the host animal, the target protein may be combined with an adjuvant, where suitable adjuvants include alum, dextran, sulfate, large polymeric anions, oil & water emulsions, e.g. Freund's adjuvant, Freund's complete adjuvant, and the like. The target protein may also be conjugated to a carrier, e.g., KLH, BSA, a synthetic carrier protein, and the like. A variety of hosts may be immunized to produce the polyclonal antibodies. Such hosts include rabbits, guinea pigs, rodents, e.g. mice, rats, sheep, goats, and the like. The target protein is administered to the host, e.g., intradermally, with an initial dosage followed by one or more, usually at least two, additional booster dosages. Following immunization, the blood from the host will be collected, followed by separation of the serum from the blood cells. The Ig present in the resultant antiserum may be further fractionated using known methods, such as ammonium salt fractionation, DEAE chromatography, and the like.

Monoclonal antibodies are produced by conventional techniques. Generally, the spleen and/or lymph nodes of an immunized host animal provide a source of plasma cells. The plasma cells are immortalized by fusion with myeloma cells to produce hybridoma cells. Culture supernatant from individual hybridomas is screened using standard techniques to identify those producing antibodies with the desired specificity. Suitable animals for production of monoclonal antibodies to the human protein include mouse, rat, hamster, etc. The antibody may be purified from the hybridoma cell supernatants or ascites fluid by conventional techniques, e.g. affinity chromatography using protein bound to an insoluble support, protein A sepharose, etc.

The antibody may be produced as a single chain, instead of the normal multimeric structure. Single chain antibodies are described in Jost et al. (1994) J. Biol. Chem. 269:26267-73, and elsewhere. DNA sequences encoding the variable region of the heavy chain and the variable region of the light chain are ligated to a spacer encoding at least about 4 amino acids of small neutral amino acids, including glycine and/or serine. The protein encoded by this fusion allows assembly of a functional variable region that retains the specificity and affinity of the original antibody.

Also provided are “artificial” antibodies, e.g., antibodies and antibody fragments produced and selected in vitro. In some embodiments, such antibodies are displayed on the surface of a bacteriophage or other viral particle. In many embodiments, such artificial antibodies are present as fusion proteins with a viral or bacteriophage structural protein, including, but not limited to, M13 gene III protein. Methods of producing such artificial antibodies are well known in the art. See, e.g., U.S. Pat. Nos. 5,516,637; 5,223,409; 5,658,727; 5,667,988; 5,498,538; 5,403,484; 5,571,698; and 5,625,033.

Also of interest are humanized antibodies. Methods of humanizing antibodies are known in the art. The humanized antibody may be the product of an animal having transgenic human immunoglobulin constant region genes (see for example International Patent Applications WO 90/10077 and WO 90/04036). Alternatively, the antibody of interest may be engineered by recombinant DNA techniques to substitute the CH1, CH2, CH3, hinge domains, and/or the framework domain with the corresponding human sequence (see WO 92/02190).

The use of Ig cDNA for construction of chimeric immunoglobulin genes is known in the art (Liu et al. (1987) Proc. Natl. Acad. Sci. USA. 84:3439 and (1987) J. Immunol. 139:3521). mRNA is isolated from a hybridoma or other cell producing the antibody and used to produce cDNA. The cDNA of interest may be amplified by the polymerase chain reaction using specific primers (U.S. Pat. Nos. 4,683,195 and 4,683,202). Alternatively, a library is made and screened to isolate the sequence of interest. The DNA sequence encoding the variable region of the antibody is then fused to human constant region sequences. The sequences of human constant regions genes may be found in Kabat et al. (1991) Sequences of Proteins of Immunological Interest, N.I.H. publication no. 91-3242. Human C region genes are readily available from known clones. The choice of isotype will be guided by the desired effector functions, such as complement fixation, or activity in antibody-dependent cellular cytotoxicity. Exemplary isotypes are IgG1, IgG3 and IgG4. Either of the human light chain constant regions, kappa or lambda, may be used. The chimeric, humanized antibody is then expressed by conventional methods. Other methods for preparing chimeric antibodies are described in, e.g., U.S. Pat. No. 5,565,332.

Antibody fragments, such as Fv, F(ab′)₂ and Fab may be prepared by cleavage of the intact protein, e.g. by protease or chemical cleavage. Alternatively, a truncated gene is designed. For example, a chimeric gene encoding a portion of the F(ab′)₂ fragment would include DNA sequences encoding the CH1 domain and hinge region of the H chain, followed by a translational stop codon to yield the truncated molecule.

Consensus sequences of H and L J regions may be used to design oligonucleotides for use as primers to introduce useful restriction sites into the J region for subsequent linkage of V region segments to human C region segments. C region cDNA can be modified by site directed mutagenesis to place a restriction site at the analogous position in the human sequence.

Expression vectors include plasmids, retroviruses, YACs, BACs; EBV-derived episomes, and the like. A convenient vector is one that encodes a functionally complete human CH or CL immunoglobulin sequence, with appropriate restriction sites engineered so that any VH or VL sequence can be easily inserted and expressed. In such vectors, splicing usually occurs between the splice donor site in the inserted J region and the splice acceptor site preceding the human C region, and also at the splice regions that occur within the human CH exons. Polyadenylation and transcription termination occur at native chromosomal sites downstream of the coding regions. The resulting chimeric antibody may be joined to any strong promoter, including retroviral long terminal repeats (LTRs) and other promoters, e.g. SV-40 early promoter, (Okayama et al. (1983) Mol. Cell. Bio. 3:280), Rous sarcoma virus LTR (Gorman et al. (1982) Proc. Natl. Acad. Sci. USA 79:6777), and moloney murine leukemia virus LTR (Grosschedl et al. (1985) Cell 41:885); native Ig promoters, etc.

Intrabodies that specifically bind RabGGT polypeptide are expressed in a cell in an individual, where they reduce levels of enzymatically active RabGGT. See, e.g., Marasco et al. (1999) J. Immunol. Methods 231:223-238. Intracellularly expressed antibodies, or intrabodies, are single-chain antibody molecules designed to specifically bind and inactivate target molecules inside cells. See, e.g., Chen et al., Hum. Gen. Ther. (1994) 5:595-601; Hassanzadeh et al., Febs Lett. (1998) 16(1, 2):75-80 and 81-86; Marasco (1997) Gene Ther. 4:11-15; and “Intrabodies: Basic Research and Clinical Gene Therapy Applications” W. A. Marasco, eg., (1998) Springer-Verlag, NY. Inducible expression vectors can be constructed that encode intrabodies that bind specifically to RabGGT polypeptide. These vectors are introduced into an individual, and production of the intrabody induced by administration to the individual of the inducer. Alternatively, the expression vector encoding the intrabody provides for constitutive production of the intrabody.

A subject antibody may be labeled. Suitable labels include radioisotopes; enzymes whose products are detectable (e.g., luciferase, β-galactosidase, and the like); fluorescent labels (e.g., fluorescein isothiocyanate, rhodamine, phycoerythrin, and the like); fluorescence emitting metals, e.g., ¹⁵²Eu, or others of the lanthanide series, attached to the antibody through metal chelating groups such as EDTA; chemiluminescent compounds, e.g., luminol, isoluminol, acridinium salts, and the like; bioluminescent compounds, e.g., luciferin, aequorin (a green fluorescent protein), and the like.

Suitable detectable moieties include, but are not limited to, fluorescent, metallic, enzymatic and radioactive markers such as fluorescent proteins, biotin, gold, ferritin, alkaline phosphatase, β-galactosidase, luciferase, horse radish peroxidase, peroxidase, urease, fluorescein, rhodamine, tritium, ¹⁴C, and iodination. The binding agent, e.g., an antibody, can be used as a fusion protein, where the fusion partner is a fluorescent protein. Fluorescent proteins include, but are not limited to, a green fluorescent protein from Aequoria victoria or a mutant or derivative thereof e.g., as described in U.S. Pat. Nos. 6,066,476; 6,020,192; 5,985,577; 5,976,796; 5,968,750; 5,968,738; 5,958,713; 5,919,445; 5,874,304; e.g., Enhanced GFP, many such GFP which are available commercially, e.g., from Clontech, Inc.; any of a variety of fluorescent and colored proteins from Anthozoan species, as described in, e.g., Matz et al. (1999) Nature Biotechnol. 17:969-973; and the like.

Nucleic Acid Modulators

In some embodiments, an agent that modulates a level of RabGGT is a nucleic acid. Nucleic acid modulators of RabGGT levels include RNAi, ribozymes, and antisense RNA.

In some embodiments, the active agent is an interfering RNA (RNAi). RNAi includes double-stranded RNA interference (dsRNAi). Use of RNAi to reduce a level of a particular mRNA and/or protein is based on the interfering properties of double-stranded RNA derived from the coding regions of gene. In one example of this method, complementary sense and antisense RNAs derived from a substantial portion of the RabGGT gene are synthesized in vitro. The resulting sense and antisense RNAs are annealed in an injection buffer, and the double-stranded RNA injected or otherwise introduced into the subject (such as in their food or by soaking in the buffer containing the RNA). See, e.g., WO99/32619. In another embodiment, dsRNA derived from a RabGGT gene is generated in vivo by simultaneous expression of both sense and antisense RNA from appropriately positioned promoters operably linked to RabGGT coding sequences in both, sense and antisense orientations.

Antisense molecules can be used to down-regulate expression of the gene encoding RabGGT in cells. Antisense compounds include ribozymes, external guide sequence (EGS) oligonucleotides (oligozymes), and other short catalytic RNAs or catalytic oligonucleotides which hybridize to the target nucleic acid and modulate its expression.

The anti-sense reagent may be antisense oligonucleotides (ODN), particularly synthetic ODN having chemical modifications from native nucleic acids, or nucleic acid constructs that express such anti-sense molecules as RNA. The antisense sequence is complementary to the mRNA of the targeted gene, and inhibits expression of the targeted gene products. Antisense molecules inhibit gene expression through various mechanisms, e.g. by reducing the amount of mRNA available for translation, through activation of RNAse H, or steric hindrance. One or a combination of antisense molecules may be administered, where a combination may comprise multiple different sequences.

Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like. It has been found that short oligonucleotides, of from 7 to 8 bases in length, can be strong and selective inhibitors of gene expression (see Wagner et al. (1996), Nature Biotechnol. 14:840-844).

A specific region or regions of the endogenous sense strand mRNA sequence is chosen to be complemented by the antisense sequence. Selection of a specific sequence for the oligonucleotide may use an empirical method, where several candidate sequences are assayed for inhibition of expression of the target gene in an in vitro or animal model. A combination of sequences may also be used, where several regions of the mRNA sequence are selected for antisense complementation.

Antisense oligonucleotides may be chemically synthesized by methods known in the art (see Wagner et al. (1993), supra, and Milligan et al., supra.) Preferred oligonucleotides are chemically modified from the native phosphodiester structure, in order to increase their intracellular stability and binding affinity. A number of such modifications have been described in the literature, which modifications alter the chemistry of the backbone, sugars or heterocyclic bases.

Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH2-5′-O-phosphonate and 3′-NH-5′-O-phosphoroamidate. Peptide nucleic acids replace the entire ribose phosphodiester backbone with a peptide linkage. Sugar modifications are also used to enhance stability and affinity. The β-anomer of deoxyribose may be used, where the base is inverted with respect to the natural α-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

Exemplary modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Oligonucleotides having a morpholino backbone structure (Summerton, J. E. and Weller D. D., U.S. Pat. No. 5,034,506) or a peptide nucleic acid (PNA) backbone (P. E. Nielson, M. Egholm, R. H. Berg, O. Buchardt, Science 1991, 254: 1497) can also be used. Morpholino antisense oligonucleotides are amply described in the literature. See, e.g., Partridge et al. (1996) Antisense Nucl. Acid Drug Dev. 6:169-175; and Summerton (1999) Biochem. Biophys. Acta 1489:141-158.

In another embodiment, the antisense oligomer is a phosphothioate morpholino oligomer (PMO). PMOs are assembled from four different morpholino subunits, each of which contain one of four genetic bases (A, C, G, or T) linked to a six-membered morpholine ring. Polymers of these subunits are joined by non-ionic phosphodiamidate intersubunit linkages. Details of how to make and use PMOs and other antisense oligomers are well known in the art (e.g. see WO99/18193; Probst J C, Antisense Oligodeoxynucleotide and Ribozyme Design, Methods. (2000) 22(3):271-281; Summerton J, and Weller D. 1997 Antisense Nucleic Acid Drug Dev. :7:187-95; U.S. Pat. No. 5,235,033; and U.S. Pat. No. 5,378,841).

As an alternative to anti-sense inhibitors, catalytic nucleic acid compounds, e.g. ribozymes, anti-sense conjugates, etc. may be used to inhibit gene expression. Ribozymes may be synthesized in vitro and administered to the patient, or may be encoded on an expression vector, from which the ribozyme is synthesized in the targeted cell (for example, see International patent application WO 9523225, and Beigelman et al. (1995), Nucl. Acids Res. 23:4434-42). Examples of oligonucleotides with catalytic activity are described in WO 9506764. Conjugates of anti-sense ODN with a metal complex, e.g. terpyridylCu(II), capable of mediating mRNA hydrolysis are described in Bashkin et al. (1995), Appl. Biochem. Biotechnol. 54:43-56.

Alternative RabGGT nucleic acid modulators are double-stranded RNA species mediating RNA interference (RNAi). RNAi is the process of sequence-specific, post-transcriptional gene silencing in animals and plants, initiated by double-stranded RNA (dsRNA) that is homologous in sequence to the silenced gene. Methods relating to the use of RNAi to silence genes in C. elegans, Drosophila, plants, and humans are known in the art (Fire A, et al., 1998 Nature 391:806-811; Fire, A. Trends Genet. 15, 358-363 (1999); Sharp, P. A. RNA interference 2001. Genes Dev. 15, 485-490 (2001); Hammond, S. M., et al., Nature Rev. Genet. 2, 110-1119 (2001); Tuschl, T. Chem. Biochem. 2, 239-245 (2001); Hamilton, A. et al., Science 286, 950-952 (1999); Hammond, S. M., et al., Nature 404, 293-296 (2000); Zamore, P. D., et al., Cell 101, 25-33 (2000); Bernstein, E., et al., Nature 409, 363-366 (2001); Elbashir, S. M., et al., Genes Dev. 15, 188-200 (2001); WO0129058; WO9932619; Elbashir S M, et al., 2001 Nature 411:494-498).

### Methods of Determining Tumor Susceptibility

In some embodiments, the present invention provides methods for determining the susceptibility of a tumor to treatment by administration of a RabGGT inhibitor. In some embodiments, the methods comprise: a) detecting a level of RabGGT protein in a cell in an individual; and b) administering to the individual an effective amount of a RabGGT modulating agent. In other embodiments, the methods comprise: a) detecting a level of RabGGT enzymatic activity in a cell in an individual; and b) administering to the individual an effective amount of a RabGGT modulating agent. In other embodiments, the methods comprise: a) detecting a level of RabGGT mRNA in a cell in an individual; and b) administering to the individual an effective amount of a RabGGT modulating agent.

Methods of detecting a level of RabGGT protein, methods of detecting a level of RabGGT enzymatic activity, and methods of detecting a level of RabGGT mRNA are described above.

In some embodiments, the methods further comprise administering an effective amount of amount of a RabGGT inhibitor to an individual having a tumor that is susceptible to treatment with a RabGGT inhibitor.

### Disorders Amenable to Treatment

Disorders amenable to treatment with the methods of the present invention include disorders associated with or caused by uncontrolled cell proliferation; disorders amenable to treatment by inducing apoptosis; and disorders associated with or caused by excessive apoptosis.

Disorders which can be treated using methods of the invention for inducing apoptosis include, but are not limited to, undesired, excessive, or uncontrolled cellular proliferation, including, for example, neoplastic cells; as well as any undesired cell or cell type in which induction of cell death is desired, e.g., virus-infected cells and self-reactive immune cells. The methods may be used to treat follicular lymphomas, carcinomas associated with p53 mutations; autoimmune disorders, such as, for example, systemic lupus erythematosus (SLE), immune-mediated glomerulonephritis; hormone-dependent tumors, such as, for example, breast cancer, prostate cancer and ovary cancer; and viral infections, such as, for example, herpesviruses, poxviruses and adenoviruses.

Disorders which can be treated using the methods of the invention for reducing apoptosis in a eukaryotic cell, include, but are not limited to, cell death associated with Alzheimer's disease, Parkinson's disease, rheumatoid arthritis, septic shock, sepsis, stroke, central nervous system inflammation, osteoporosis, ischemia, reperfusion injury, cell death associated with cardiovascular disease, polycystic kidney disease, cell death of endothelial cells in cardiovascular disease, degenerative liver disease, multiple sclerosis, amyotropic lateral sclerosis, cerebellar degeneration, ischemic injury, cerebral infarction, myocardial infarction, acquired immunodeficiency syndrome (AIDS), myelodysplastic syndromes, aplastic anemia, male pattern baldness, and head injury damage. Also included are conditions in which DNA damage to a cell is induced by, e.g., irradiation, radiomimetic drugs, and the like. Also included are any hypoxic or anoxic conditions, e.g., conditions relating to or resulting from ischemia, myocardial infarction, cerebral infarction, stroke, bypass heart surgery, organ transplantation, neuronal damage, and the like.

Cancer

Generally, cells in a benign tumor retain their differentiated features and do not divide in a completely uncontrolled manner. A benign tumor is usually localized and nonmetastatic. Specific types benign tumors that can be treated using the present invention include hemangiomas, hepatocellular adenoma, cavernous haemangioma, focal, nodular hyperplasia, acoustic neuromas, neurofibroma, bile duct adenoma, bile duct cystanoma, fibroma, lipomas, leiomyomas, mesotheliomas, teratomas, myxomas, nodular regenerative hyperplasia, trachomas and pyogenic granulomas.

In a malignant tumor cells become undifferentiated, do not respond to the body's growth control signals, and multiply in an uncontrolled manner. The malignant tumor is invasive and capable of spreading to distant sites (metastasizing). Malignant tumors are generally divided into two categories: primary and secondary. Primary tumors arise directly from the tissue in which they are found. A secondary tumor, or metastasis, is a tumor which originated elsewhere in the body but has now spread to a distant organ. The common routes for metastasis are direct growth into adjacent structures, spread through the vascular or lymphatic systems, and tracking along tissue planes and body spaces (peritoneal fluid, cerebrospinal fluid, etc.)

Specific types of cancers or malignant tumors, either primary or secondary, that can be treated using this invention include leukemia, breast cancer, skin cancer, bone cancer, prostate cancer, liver cancer, lung cancer, brain cancer, cancer of the larynx, gallbladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteosarcoma, Ewing's sarcoma, veticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuromas, intestinal ganglioneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythermia vera, adenocarcinoma, glioblastoma multiforme, leukemias, lymphomas, malignant melanomas, epidermoid carcinomas, and other carcinomas and sarcomas.

Subjects to be treated according to the methods of the invention include any individual having any of the above-mentioned disorders. Further included are individuals who are at risk of developing any of the above-mentioned disorders, including, but not limited to, an individual who has suffered a myocardial infarction, and is therefore at risk for experiencing a subsequent myocardial infarction; an individual who has undergone organ or tissue transplantation; an individual who has had a stroke and is at risk for having a subsequent stroke; and an individual at risk of developing an autoimmune disorder due to genetic predisposition, or due to the appearance of early symptoms of autoimmune disorder.

Determining Efficacy of Treatment

Whether a tumor load has been decreased can be determined using any known method, including, but not limited to, measuring solid tumor mass; counting the number of tumor cells using cytological assays; fluorescence-activated cell sorting (e.g., using antibody specific for a tumor-associated antigen); computed tomography scanning, magnetic resonance imaging, and/or x-ray imaging of the tumor to estimate and/or monitor tumor size; measuring the amount of tumor-associated antigen in a biological sample, e.g., blood; and the like.

### Formulations, Dosages, and Routes of Administration

Formulations

An agent that modulates a level and/or activity of RabGGT may be formulated in a variety of ways. For example, and agent may include a buffer, which is selected according to the desired use of the agent, and may also include other substances appropriate to the intended use. Those skilled in the art can readily select an appropriate buffer, a wide variety of which are known in the art, suitable for an intended use. In some instances, the composition can comprise a pharmaceutically acceptable excipient, a variety of which are known in the art and need not be discussed in detail herein. Pharmaceutically acceptable excipients have been amply described in a variety of publications, including, for example, “Remington: The Science and Practice of Pharmacy”, 19^{th} Ed. (1995), or latest edition, Mack Publishing Co; A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3_{rd} ed. Amer. Pharmaceutical Assoc.

In the subject methods, the active agent(s) may be administered to the host using any convenient means capable of resulting in the desired modulation in a level and/or an activity of RabGGT. Thus, the agent can be incorporated into a variety of formulations for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The agents can be utilized in aerosol formulation to be administered via inhalation. The compounds of the present invention can be formulated into pressurized acceptable propellants such as dichlorodifluoromethane, propane, nitrogen and the like.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle.

The specifications for the novel unit dosage forms of the present invention depend on the particular compound employed and the effect to be achieved, and the pharmacodynamics associated with each compound in the host.

Other modes of administration will also find use with the subject invention. For instance, an agent of the invention can be formulated in suppositories and, in some cases, aerosol and intranasal compositions. For suppositories, the vehicle composition will include traditional binders and carriers such as, polyalkylene glycols, or triglycerides. Such suppositories may be formed from mixtures containing the active ingredient in the range of about 0.5% to about 10% (w/w), preferably about 1% to about 2%.

Intranasal formulations will usually include vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of the subject proteins by the nasal mucosa.

An agent of the invention can be administered as injectables. Typically, injectable compositions are prepared as liquid solutions or suspensions; solid forms suitable for solution in, or suspension in, liquid vehicles prior to injection may also be prepared. The preparation may also be emulsified or the active ingredient encapsulated in liposome vehicles.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pa., 17th edition, 1985. The composition or formulation to be administered will, in any event, contain a quantity of the agent adequate to achieve the desired state in the subject being treated.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Dosages

Although the dosage used will vary depending on the clinical goals to be achieved, a suitable dosage range is one which provides up to about 1 μg to about 1,000 μg or about 10,000 μg of an agent that reduces a level and/or an activity of RabGGT can be administered in a single dose. Alternatively, a target dosage of an agent that modulates a level and/or an activity of RabGGT can be considered to be about in the range of about 0.1-1000 μM, about 0.5-500 μM, about 1-100 μM, or about 5-50 μM in a sample of host blood drawn within the first 24-48 hours after administration of the agent.

Those of skill will readily appreciate that dose levels can vary as a function of the specific compound, the severity of the symptoms and the susceptibility of the subject to side effects. Preferred dosages for a given compound are readily determinable by those of skill in the art by a variety of means.

Routes of Administration

An agent that modulates a level and/or activity of RabGGT may be administered (including self-administered) orally, parenterally, intraperitoneally, intravenously, intraarterially, transdermally, intratumorally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally.

An agent that modulates a level and/or activity of RabGGT may be administered by a variety of routes, and may be administered in any conventional dosage form. In some embodiments, an agent that modulates a level and/or activity of RabGGT is administered in combination therapy (e.g., is “coadministered) with at least a second therapeutic agent. Coadministration in the context of this invention is defined to mean the administration of more than one therapeutic in the course of a coordinated treatment to achieve an improved clinical outcome. Such coadministration may also be coextensive, that is, occurring during overlapping periods of time.

One route of administration or coadministration is local delivery. Local delivery of an effective amount of an agent that modulates an activity and/or level of RabGGT can be by a variety of techniques and devices that administer the agent(s) at or near a desired site. Examples of local delivery techniques and structures are not intended to be limiting but rather as illustrative of the techniques and structures available. Examples include local delivery catheters, site specific carriers, implants, direct injection, or direct applications.

Local delivery by a catheter allows the administration of an agent directly to the desired site. Examples of local delivery using a balloon catheter are described in EP 383 492 A2 and U.S. Pat. No. 4,636,195 to Wolinsky. Additional examples of local, catheter-based techniques and structures are disclosed in U.S. Pat. No. 5,049,132 to Shaffer et al. and U.S. Pat No. 5,286,254 to Shapland et al.

Generally, the catheter must be placed such that the agent is delivered at or near the desired site. Dosages delivered through the catheter can vary, according to determinations made by one of skill, but often are in amounts effective to generate the desired effect at the local site. Preferably, these total amounts are less than the total amounts for systemic administration of an agent, and are less than the maximum tolerated dose. The agent(s) delivered through catheters is generally formulated in a viscosity that enables delivery through a small treatment catheter, and may be formulated with pharmaceutically acceptable additional ingredients (active and inactive).

Local delivery by an implant describes the placement of a matrix that contains an agent into the desired site. The implant may be deposited by surgery or other means. The implanted matrix releases the agent by diffusion, chemical reaction, solvent activators, or other equivalent mechanisms. Examples are set forth in Lange, Science 249:1527-1533 (September, 1990). Often the implants may be in a form that releases the agent over time; these implants are termed time-release implants. The material of construction for the implants will vary according to the nature of the implant and the specific use to which it will be put. For example, biostable implants may have a rigid or semi-rigid support structure, with agent delivery taking place through a coating or a porous support structure. Other implants made be made of a liquid that stiffens after being implanted or may be made of a gel. The amounts of agent present in or on the implant may be in an amount effective to treat cell proliferation generally, or a specific proliferation indication, such as the indications discussed herein. One example of local delivery of an agent by an implant is use of a biostable or bioabsorbable plug or patch or similar geometry that can deliver the agent once placed in or near the desired site.

A non-limiting example of local delivery by an implant is the use of a stent. Stents are designed to mechanically prevent the collapse and reocclusion of the coronary arteries. Incorporating an agent into the stent may deliver the agent directly to or near the proliferative site. Certain aspects of local delivery by such techniques and structures are described in Kohn, Pharmaceutical Technology (October, 1990). Stents may be coated with the agent to be delivered. Examples of such techniques and structures may be found in U.S. Pat. No. 5,464,650 to Berg et al., U.S. Pat. No. 5,545,208 to Wolff et al., U.S. Pat. No. 5,649,977 to Campbell, U.S. Pat. No. 5,679,400 to Tuch, EP 0 716 836 to Tartaglia et al. Alternatively, the agent-loaded stent may be bioerodable, i.e. designed to dissolve, thus releasing the agent in or near the desired site, as disclosed in U.S. Pat. No. 5,527,337 to Stack et al. The present invention can be used with a wide variety of stent configurations, including, but not limited to shape memory alloy stents, expandable stents, and stents formed in situ.

Another example is a delivery system in which a polymer that contains an agent is injected into the target cells in liquid form. The polymer then cures to form the implant in situ. One variation of this technique and structure is described in WO 90/03768.

Another example is the delivery of an agent by polymeric endoluminal sealing. This technique and structure uses a catheter to apply a polymeric implant to the interior surface of the lumen. The agent incorporated into the biodegradable polymer implant is thereby released at the desired site. One example of this technique and structure is described in WO 90/01969.

Another example of local delivery by an implant is by direct injection of vesicles or microparticulates into the desired site. These microparticulates may comprise substances such as proteins, lipids, carbohydrates or synthetic polymers. These microparticulates have an agent incorporated throughout the microparticle or over the microparticle as a coating. Examples of delivery systems incorporating microparticulates are described in Lange, Science, 249:1527-1533 (September, 1990) and Mathiowitz, et al., J. App. Poly Sci. 26:809 (1981).

Local delivery by site specific carriers may involve linking an agent to a carrier which will direct the drug to the desired site. Examples of this delivery technique and structure include the use of carriers such as a protein ligand or a monoclonal antibody. Certain aspects of these techniques and structures are described in Lange, Science 249:1527-1533.

Local delivery also includes the use of topical applications. An example of a local delivery by topical application is applying an agent directly to an arterial bypass graft during a surgical procedure. Other equivalent examples will no doubt occur to one of skill in the art.

Combination Therapies

An agent that reduces the level and/or activity of RabGGT may be administered in combination therapy with one or more additional therapeutic agents.

An agent that reduces the level and/or activity of RabGGT may be administered in combination therapy with one or more antiangiogenesis agents to inhibit undesirable and uncontrolled angiogenesis. Examples of anti-angiogenesis agents include, but are not limited to, retinoid acid and derivatives thereof, 2-methoxyestradiol, ANGIOSTATIN™ protein, ENDOSTATIN™ protein, suramin, squalamine, tissue inhibitor of metalloproteinase-I, tissue inhibitor of metalloproteinase-2, plasminogen activator inhibitor-1, plasminogen activator inhibitor-2, cartilage-derived inhibitor, paclitaxel, platelet factor 4, protamine sulphate (clupeine), sulfated chitin derivatives, sulfated polysaccharide peptidoglycan complex (sp-pg), staurosporine, modulators of matrix metabolism, including for example, proline analogs ((I-azetidine-2-carboxylic acid (LACA), cishydroxyproline, d,I-3,4-dehydroproline, thiaproline], α, α-dipyridyl, β-aminopropionitrile fumarate, 4-propyl-5-(4-pyridinyl)-2(3h)-oxazolone; methotrexate, mitoxantrone, heparin, interferons, 2 macroglobulin-serum, chimp-3, chymostatin, β-cyclodextrin tetradecasulfate, eponemycin; fumagillin, gold sodium thiomalate, d-penicillamine (CDPT), β-1-anticollagenase-serum, α2-antiplasmin, bisantrene, lobenzarit disodium, n-(2-carboxyphenyl-4-chloroanthronilic acid disodium or “CCA”, thalidomide; angostatic steroid, cargboxynaminolmidazole; metalloproteinase inhibitors such as BB94. Other anti-angiogenesis agents include antibodies, e.g., monoclonal antibodies against these angiogenic growth factors: bFGF, aFGF, FGF-5, VEGF isoforms, VEGF-C, HGF/SF and Ang-1/Ang-2. Ferrara N. and Alitalo, K. “Clinical application of angiogenic growth factors and their inhibitors” (1999) Nature Medicine 5:1359-1364.

An agent that reduces the level and/or activity of RabGGT may be administered in combination therapy with one or more antiproliferative agents, or as an adjuvant to a standard cancer treatment. Standard cancer therapies include surgery (e.g., surgical removal of cancerous tissue), radiation therapy, bone marrow transplantation, chemotherapeutic treatment, biological response modifier treatment, and certain combinations of the foregoing.

Radiation therapy includes, but is not limited to, x-rays or gamma rays that are delivered from either an externally applied source such as a beam, or by implantation of small radioactive sources.

Chemotherapeutic agents are non-peptidic (i.e., non-proteinaceous) compounds that reduce proliferation of cancer cells, and encompass cytotoxic agents and cytostatic agents. Non-limiting examples of chemotherapeutic agents include alkylating agents, nitrosoureas, antimetabolites, antitumor antibiotics, plant (vinca) alkaloids, and steroid hormones.

Agents that act to reduce cellular proliferation are known in the art and widely used. Such agents include alkylating agents, such as nitrogen mustards, nitrosoureas, ethylenimine derivatives, alkyl sulfonates, and triazenes, including, but not limited to, mechlorethamine, cyclophosphamide (Cytoxan™), melphalan (L-sarcolysin), carmustine (BCNU), lomustine (CCNU), semustine (methyl-CCNU), streptozocin, chlorozotocin, uracil mustard, chlormethine, ifosfamide, chlorambucil, pipobroman, triethylenemelamine, triethylenethiophosphoramine, busulfan, dacarbazine, and temozolomide.

Antimetabolite agents include folic acid analogs, pyrimidine analogs, purine analogs, and adenosine deaminase inhibitors, including, but not limited to, cytarabine (CYTOSAR-U), cytosine arabinoside, fluorouracil (5-FU), floxuridine (FudR), 6-thioguanine, 6-mercaptopurine (6-MP), pentostatin, 5-fluorouracil (5-FU), methotrexate, 10-propargyl-5,8-dideazafolate (PDDF, CB3717), 5,8-dideazatetrahydrofolic acid (DDATHF), leucovorin, fludarabine phosphate, pentostatine, and gemeitabine.

Suitable natural products and their derivatives, (e.g., vinca alkaloids, antitumor antibiotics, enzymes, lymphokines, and epipodophyllotoxins), include, but are not limited to, Ara-C, paclitaxel (Taxol®), docetaxel (Taxotere®), deoxycoformycin, mitomycin-C, L-asparaginase, azathioprine; brequinar; alkaloids, e.g. vincristine, vinblastine, vinorelbine, vindesine, etc.; podophyllotoxins, e.g. etoposide, teniposide, etc.; antibiotics, e.g. anthracycline, daunorubicin hydrochloride (daunomycin, rubidomycin, cerubidine), idarubicin, doxorubicin, epirubicin and morpholino derivatives, etc.; phenoxizone biscyclopeptides, e.g. dactinomycin; basic glycopeptides, e.g. bleomycin; anthraquinone glycosides, e.g. plicamycin (mithramycin); anthracenediones, e.g mitoxantrone; azirinopyrrolo indolediones, e.g. mitomycin; macrocyclic immunosuppressants, e.g. cyclosporine, FK-506 (tacrolimus, prograf), rapamycin, etc.; and the like.

Other anti-proliferative cytotoxic agents are navelbene, CPT-11, anastrazole, letrazole, capecitabine, reloxafine, cyclophosphamide, ifosamide, and droloxafine.

Microtubule affecting agents that have antiproliferative activity are also suitable for use and include, but are not limited to, allocolchicine (NSC 406042), Halichondrin B (NSC 609395), colchicine (NSC 757), colchicine derivatives (e.g., NSC 33410), dolstatin 10 (NSC 376128), maytansine (NSC 153858), rhizoxin (NSC 332598), paclitaxel (Taxol™), Taxol™ derivatives, docetaxel (Taxotere™), thiocolchicine (NSC 361792), trityl cysterin, vinblastine sulfate, vincristine sulfate, natural and synthetic epothilones including but not limited to, eopthilone A, epothilone B, discodermolide; estramustine, nocodazole, and the like.

Hormone modulators and steroids (including synthetic analogs) that are suitable for use include, but are not limited to, adrenocorticosteroids, e.g. prednisone, dexamethasone, etc.; estrogens and pregestins, e.g. hydroxyprogesterone caproate, medroxyprogesterone acetate, megestrol acetate, estradiol, clomiphene, tamoxifen; etc.; and adrenocortical suppressants, e.g. aminoglutethimide; 17α-ethinylestradiol; diethylstilbestrol, testosterone, fluoxymesterone, dromostanolone propionate, testolactone, methylprednisolone, methyl-testosterone, prednisolone, triamcinolone, chlorotrianisene, hydroxyprogesterone, aminoglutethimide, estramustine, medroxyprogesterone acetate, leuprolide, Flutamide (Drogenil), Toremifene (Fareston), and Zoladex™. Estrogens stimulate proliferation and differentiation, therefore compounds that bind to the estrogen receptor are used to block this activity. Corticosteroids may inhibit T cell proliferation.

Other chemotherapeutic agents include metal complexes, e.g. cisplatin (cis-DDP), carboplatin, etc.; ureas, e.g. hydroxyurea; and hydrazines, e.g. N-methylhydrazine; epidophyllotoxin; a topoisomerase inhibitor; procarbazine; mitoxantrone; leucovorin; tegafur; etc. Other anti-proliferative agents of interest include immunosuppressants, e.g. mycophenolic acid, thalidomnide, desoxyspergualin, azasporine, leflunomide, mizoribine, azaspirane (SKF 105685); Iressa® (ZD 1839, 4-(3-chloro-4-fluorophenylamino)-7-methoxy-6-(3-(4-morpholinyl)propoxy)quinazoline); etc.

“Taxanes” include paclitaxel, as well as any active taxane derivative or pro-drug. “Paclitaxel” (which should be understood herein to include analogues, formulations, and derivatives such as, for example, docetaxel, TAXOL™, TAXOTERE™ (a formulation of docetaxel), 10-desacetyl analogs of paclitaxel and 3′N-desbenzoyl-3′N-t-butoxycarbonyl analogs of paclitaxel) may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267), or obtained from a variety of commercial sources, including for example, Sigma Chemical Co., St. Louis, Mo. (T7402 from Taxus brevifolia; or T-1912 from Taxus yannanensis).

Paclitaxel should be understood to refer to not only the common chemically available form of paclitaxel, but analogs and derivatives (e.g., Taxotere™ docetaxel, as noted above) and paclitaxel conjugates (e.g., paclitaxel-PEG, paclitaxel-dextran, or paclitaxel-xylose).

Also included within the term “taxane” are a variety of known derivatives, including both hydrophilic derivatives, and hydrophobic derivatives. Taxane derivatives include, but not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; and taxol derivative described in U.S. Pat. No. 5,415,869. It further includes prodrugs of paclitaxel including, but not limited to, those described in WO 98/58927; WO 98/13059; and U.S. Pat. No. 5,824,701.

Biological response modifiers suitable for use in connection with the methods of the invention include, but are not limited to, (1) inhibitors of tyrosine kinase (RTK) activity; (2) inhibitors of serine/threonine kinase activity; (3) tumor-associated antigen antagonists, such as antibodies that bind specifically to a tumor antigen; (4) apoptosis receptor agonists; (5) interleukin-2; (6) IFN-α; (7) IFN-γ (8) colony-stimulating factors; (9) inhibitors of angiogenesis; and (10) antagonists of tumor necrosis factor.

### Screening Methods

The present invention provides methods of identifying an agent that induces apoptosis and/or inhibits cell proliferation. The method comprises screening a test agent in an assay system that detects changes in RabGGT level or activity. Any of the methods previously discussed for determining RagGGT protein level, RabGGT mRNA level, RabGGT enzymatic activity, RabGGT binding activity, etc. can be used in the assay system. For the discovery of small molecule modulators, the assay system may employ high-throughput screening of a combinatorial library. A small molecule that is identified as reducing RabGGT levels or activity is then further tested to determine whether it induces apoptosis in a cell and/or inhibit cell proliferation. In an alternative embodiment, a compound already known to induce apoptosis and/or inhibit cell proliferation may serve as the test agent to determine whether the mechanism of action of the compound is through targeting RabGGT. A compound identified as inhibiting RabGGT activity and having an apoptotic and/or anti-proliferative effect on cells may serve as a “lead compound” from which further “analog compounds” are designed and synthesized in a drug development/optimization process to improve structure-activity relationship and other properties such as absorption, distribution, metabolism and excretion (ADME), etc. Typically, the analog compounds are synthesized to have an electronic configuration and a molecular conformation similar to that of the lead compound.

Identification of analog compounds can be performed through use of techniques such as self-consistent field (SCF) analysis, configuration interaction (CI) analysis, and normal mode dynamics analysis. Computer programs for implementing these techniques are available. See, e.g., Rein et al., (1989) Computer-Assisted Modeling of Receptor-Ligand Interactions (Alan Liss, New York). Once analogs have been prepared, they can be screened using the methods disclosed herein to identify those analogs that exhibit an increased ability to modulate RabGGT activity. Such compounds can then be subjected to further analysis to identify those compounds that have the greatest potential as pharmaceutical agents. Alternatively, analogs shown to have activity through the screening methods can serve as lead compounds in the preparation of still further analogs, which can be screened by the methods described herein. The cycle of screening, synthesizing analogs and re-screening can be repeated multiple times.

Compounds identified as having the greatest potential as pharmaceutical agents are identified as “clinical compounds” and their safety and efficacy are further evaluated in clinical trials. Kits may be prepared comprising a clinical compound and instructions for administering the clinical compound to a patient afflicted with a disorder associated with undesired or uncontrolled cell proliferation.

The present invention further provides methods of identifying agents that selectively modulate a level and/or an activity, e.g., an enzymatic activity, of RabGGT. The present invention further provides methods of identifying agents that selectively modulate a level and/or activity of a RabGGT/REP complex.

An agent that selectively modulates a level and/or an enzymatic activity of RabGGT is an agent that does not substantially modulate a level or an enzymatic activity of another (non-RabGGT) enzyme, including farnesyl transferase, e.g., the agent modulates the level or activity of another enzyme by less than about 10%, less than about 5%, less than about 2%, or less than about 1%, compared to the activity the enzyme in the absence of the agent. Thus, in some embodiments, an agent that selectively modulates a level and/or an enzymatic activity of RabGGT modulates the activity of a farnesyl transferase by less than about 10%, less than about 5%, less than about 2%, or less than about 1%, compared to the level or the activity the farnesyl transferase in the absence of the agent. An agent that selectively modulates the level and/or enzymatic activity of RabGGT is suitable for use in a method of the present invention.

Certain screening methods involve screening for a compound that modulates the expression of the RabGGT gene. Such methods generally involve conducting cell-based assays in which test compounds are contacted with one or more cells expressing RabGGT and then detecting an increase in RabGGT gene expression (either transcript or translation product). Some assays are performed with cells that express endogenous RabGGT. Other expression assays are conducted with cells that do not express endogenous RabGGT, but that express an exogenous RabGGT sequence.

RabGGT expression can be detected in a number of different ways. The expression level of a RabGGT in a cell can be determined by probing the mRNA expressed in a cell with a probe that specifically hybridizes with a transcript (or complementary nucleic acid derived therefrom) of RabGGT. Probing can be conducted by lysing the cells and conducting Northern blots or without lysing the cells using in situ-hybridization techniques. Alternatively, RabGGT protein can be detected using immunological methods in which a cell lysate is probe with antibodies that specifically bind to RabGGT protein.

Other cell-based assays are reporter assays conducted with cells that do not express RabGGT. Certain of these assays are conducted with a heterologous nucleic acid construct that includes a RabGGT promoter that is operably linked to a reporter gene that encodes a detectable product. A number of different reporter genes can be utilized. Some reporters are inherently detectable. An example of such a reporter is green fluorescent protein that emits fluorescence that can be detected with a fluorescence detector. Other reporters generate a detectable product. Often such reporters are enzymes. Exemplary enzyme reporters include, but are not limited to, β-glucuronidase, CAT (chloramphenicol acetyl transferase; Alton and Vapnek (1979) Nature 282:864-869), luciferase, β-galactosidase and alkaline phosphatase (Toh, et al. (1980) Eur. J. Biochem. 182:231-238; and Hall et al. (1983) J. Mol. Appl. Gen. 2:101).

In these assays, cells harboring the reporter construct are contacted with a test compound. A test compound that either activates the promoter by binding to it or triggers a cascade that produces a molecule that activates the promoter causes expression of the detectable reporter. Certain other reporter assays are conducted with cells that harbor a heterologous construct that includes a transcriptional control element that activates expression of RabGGT and a reporter operably linked thereto. Here, too, an agent that binds to the transcriptional control element to activate expression of the reporter or that triggers the formation of an agent that binds to the transcriptional control element to activate reporter expression, can be identified by the generation of signal associated with reporter expression.

The level of expression or activity can be compared to a baseline value. As indicated above, the baseline value can be a value for a control sample or a statistical value that is representative of RabGGT expression levels for a control population (e.g., healthy individuals not at risk for neurological injury such as stroke). Expression levels can also be determined for cells that do not express a RabGGT as a negative control. Such cells generally are otherwise substantially genetically the same as the test cells.

A variety of different types of cells can be utilized in the reporter assays. In general, eukaryotic cells are used. The eukaryotic cells can be any of the cells typically utilized in generating cells that harbor recombinant nucleic acid constructs. Exemplary eukaryotic cells include, but are not limited to, yeast, and various higher eukaryotic cells such as the COS, CHO and HeLa cell lines.

Various controls can be conducted to ensure that an observed activity is authentic including running parallel reactions with cells that lack the reporter construct or by not contacting a cell harboring the reporter construct with test compound. Compounds can also be further validated as described below.

Compounds that are initially identified by any of the foregoing screening methods can be further tested to validate the apparent activity. The basic format of such methods involves administering a lead compound identified during an initial screen to a non-human animal that serves as a model for humans and then determining if a RabGGT activity is in fact modulated. The non-human animal models utilized in validation studies generally are mammals. Specific examples of suitable animals include, but are not limited to, primates, mice, and rats.

The present invention provides a method for identifying an agent that selectively modulates the enzymatic activity of a RabGGT enzyme, the method generally involving measuring the enzymatic activity of a RabGGT enzyme in the presence of a test agent; and measuring the enzymatic activity of a famesyl transferase enzyme in the presence of the test agent. A test agent that modulates the enzymatic activity of the RabGGT enzyme, and that does not substantially modulate the enzymatic activity of the farnesyl transferase enzyme, is considered to selectively modulate the enzymatic activity of the RabGGT enzyme. In general, a test ageni that modulates the enzymatic activity of RabGGT by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, or more, compared to the RabGGT enzymatic activity in the absence of the agent, and that modulates the enzymatic activity of the farnesyl transferase activity by less than about 10%, less than about 5%, less than about 2%, or less than about 1%, compared to the activity the farnesyl transferase in the absence of the agent, is considered to selectively modulate the enzymatic activity of the RabGGT enzyme.

The enzymatic activity of RabGGT can be determined using any known method. For example, RabGGT enzymatic activity is quantified using a filter binding assay that measures the transfer of (³H) geranylgeranyl groups (GG) from all-trans-(³H)geranylgeranyl pyrophosphate (³H-GGPP) to recombinant Rab3A protein (Shen and Seabra (1996) J. Biol. Chem. 271:3692; Armstrong et al. (1996) Methods in Enzymology 257:30), or as described in the Examples.

The enzymatic activity of farnesyl transferase can be measured using any known method, e.g., the method described in Mann et al. (1995) Drug Dev. Res. 34:121, or in Ding et al. (1999) J. Med. Chem. 42:5241.

The terms “candidate agent,” “test agent,” “agent”, “substance” and “compound” are used interchangeably herein. Candidate agents encompass numerous chemical classes, typically synthetic, semi-synthetic, or naturally-occurring inorganic or organic molecules. Candidate agents include those found in large libraries of synthetic or natural compounds. For example, synthetic compound libraries are commercially available from Maybridge Chemical Co. (Trevillet, Cornwall, UK), ComGenex (South San Francisco, Calif.), and MicroSource (New Milford, Conn.). A rare chemical library is available from Aldrich (Milwaukee, Wis.). Alternatively, libraries of natural compounds in the form of bacterial, fungal, plant and animal extracts are available from Pan Labs (Bothell, Wash.) or are readily producible.

Candidate agents may be small organic or inorganic compounds having a molecular weight of more than 50 and less than about 2,500 daltons. Candidate agents may comprise functional groups necessary for structural interaction with proteins, particularly hydrogen bonding, and may include at least an amine, carbonyl, hydroxyl or carboxyl group, and may contain at least two of the functional chemical groups. The candidate agents may comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Of particular interest are agents that inhibit the enzymatic activity of RabGGT and that induce apoptosis in a cell. Thus, in some embodiments, the methods involve: a) measuring the enzymatic activity of a RabGGT enzyme in the presence of a test agent; b) measuring the enzymatic activity of a farnesyl transferase enzyme in the presence of the test agent; and c) determining whether the test agent induces apoptosis in a eukaryotic cell.

A test agent that (1) reduces the enzymatic activity of RabGGT by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, or more, compared to the RabGGT enzymatic activity in the absence of the agent; (2) reduces the enzymatic activity of the farnesyl transferase activity by less than about 10%, less than about 5%, less than about 2%, or less than about 1%, compared to the activity the farnesyl transferase in the absence of the agent; and (3) induces apoptosis in a eukaryotic cell is considered to be a candidate agent for the treatment of disorders amenable to treatment by inducing apoptosis, as described above.

Whether a given agent inhibits RabGGT and induces apoptosis in a eukaryotic cell can be determined using any known method. Assays can be conducted on cell populations or an individual cell, and include morphological assays and biochemical assays. A non-limiting example of a method of determining the level of apoptosis in a cell population is TUNEL (TdT-mediated dUTP nick-end labeling) labeling of the 3′-OH free end of DNA fragments produced during apoptosis (Gavrieli et al. (1992) J. Cell Biol. 119:493). The TUNEL method consists of catalytically adding a nucleotide, which has been conjugated to a chromogen system or a to a fluorescent tag, to the 3′-OH end of the 180-bp (base pair) oligomer DNA fragments in order to detect the fragments. The presence of a DNA ladder of 180-bp oligomers is indicative of apoptosis. Procedures to detect cell death based on the TUNEL method are available commercially, e.g., from Boehringer Mannheim (Cell Death Kit) and Oncor (Apoptag Plus). Another marker that is currently available is annexin, sold under the trademark APOPTEST™. This marker is used in the “Apoptosis Detection Kit,” which is also commercially available, e.g., from R&D Systems. During apoptosis, a cell membrane's phospholipid asymmetry changes such that the phospholipids are exposed on the outer membrane. Annexins are a homologous group of proteins that bind phospholipids in the presence of calcium. A second reagent, propidium iodide (PI), is a DNA binding fluorochrome. When a cell population is exposed to both reagents, apoptotic cells stain positive for annexin and negative for PI, necrotic cells stain positive for both, live cells stain negative for both. Other methods of testing for apoptosis are known in the art and can be used, including, e.g., the method disclosed in U.S. Pat. No. 6,048,703.

### RabGGT Structure

The present invention provides a three-dimensional (3-D) structure of RabGGT. A 3-D structure of a RabGGT is useful for predicting whether a given compound will bind to RabGGT, and is therefore useful for determining whether a given compound will modulate an activity of RabGGT. As discussed above, agents that modulate an activity of RabGGT are useful for the treatment of various disorders. Thus, a 3-D structure of RabGGT is useful for identifying agents that are useful for the treatment of disorders, as described herein.

The subject homology model is useful for drug design; for determining whether a given compound will modulate a RabGGT activity; and for determining whether a given compound will preferentially modulate a RabGGT activity, e.g., whether a compound will modulate a RabGGT activity, but will substantially not modulate an FT activity. Accordingly, in some embodiments, the present invention provides methods for identifying agents that modulate a RabGGT activity, but that do not substantially modulate an FT activity.

The subject 3-D structure is useful for structure-based drug design. Three dimensional structural information is useful to specify the characteristics of peptides and small molecules that might bind to or mimic a target of interest. These descriptors may then be used to search small molecule databases and to establish constraints for use in the design of combinatorial libraries. Accordingly, in some embodiments, the invention provides a method for structure-based drug design, the method comprising positioning a test compound in a subject 3-D structure of RabGGT; and modifying the test compound such that the fit within a target binding site within the 3-D structure is increased.

Target binding sites within the RabGGT 3-D structure include a Rab binding site; a prenyl moiety binding site; a REP binding site; and the like. A non-limiting example of a target binding site is a Rab binding pocket of human RabGGT. The Rab binding pocket of human RabGGT contains a bound Zn atom, coordinated by His B290, Cys B240, and Asp B238; the floor of the pocket is composed of Phe B289, Trp B52; and the back of the pocket is composed of Leu B45, Ser B48, and Tyr B44.

A test compound is positioned, using computer modeling, within the 3-D structure of RabGGT using any known program. A non-limiting example of a suitable program is Insight (Accelrys, San Diego, Calif.), as described in Example XIV. In these embodiments, positioning of a test compound within a binding site of the RabGGT 3-D structure is accomplished using a computer-generated model of the structure of the test compound. The computer-generated model of the test structure is positioned within the binding site of the RabGGT 3-D structure by rotating the structure until the best fit is achieved.

To arrive at the best fit within the active site, the structure of the test compound is altered using computer modeling. As such, the invention provides a method for rational drug design, comprising positioning a test compound within a 3-D structure of RabGGT; and altering, by computer modeling, the structure of the test compound, such that the altered test compound has an enhanced fit within the binding site of the RabGGT 3-D structure. In some embodiments, a test agent is modeled within the FT structure; and agents that modulate RabGGT activity, but that do not substantially modulate FT enzymatic activity, are identified and/or designed.

In some embodiments, rational drug design using computer modeling is carried out in conjunction with in vitro testing of the test compound, and/or the altered test compound. Thus, the present invention provides a method of identifying an agent that modulates RabGGT enzymatic activity, the method comprising selecting a test agent by performing rational drug design with a subject 3-D structure of RabGGT, wherein the selecting is performed in conjunction with computer modeling; and measuring the enzymatic activity of a RabGGT polypeptide contacted in vitro with the test agent. In some of these embodiments, the activity of the test compound and/or the altered test compound are further tested for their effect on FT enzymatic activity. In other embodiments, the activity of the test compound and/or the altered test compound are further tested for their effect on apoptosis.

In some embodiments, the invention provides methods of designing a compound such that it modulates an activity of RabGGT, but does not substantially modulate an activity of an FT. In some embodiments, the invention provides methods of identifying a compound that modulates an activity of RabGGT and that does not substantially modulate an activity of an FT.

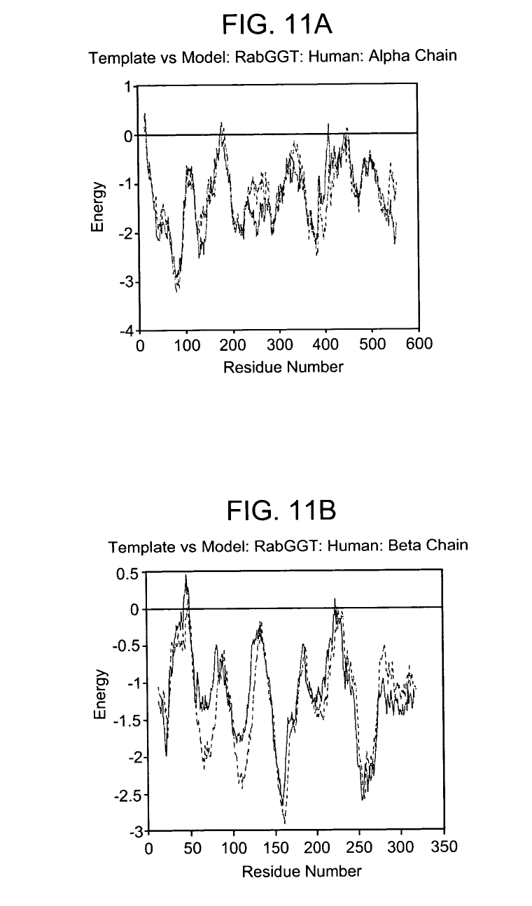

A 3-D model (“homology model”) of RabGGT was generated by homology modeling, as described in Example XIII and Example IV, and presented in FIGS. 11-15. The program LOOK was used for alignments, and the model-building module within LOOK, SEGMOD, was used to build the homology models. The 3-D model includes a model of the binding pocket for modulators of RabGGT enzymatic activity. The structure information may be provided in a computer readable form, e.g. as a database of atomic coordinates, or as a three-dimensional model. The present invention provides three-dimensional coordinates for the RabGGT structure. Such a data set may be provided in computer readable form. Methods of using such coordinates (including in computer readable form) in drug assays and drug screens as exemplified herein, are also part of the present invention. In a particular embodiment of this type, the coordinates contained in the data set of can be used to identify potential modulators of the RabGGT polypeptide.

In one embodiment, a potential agent for modulation of RabGGT is selected by performing rational drug design with the three-dimensional coordinates provided herein. Typically, the selection is performed in conjunction with computer modeling. The potential agent is then contacted with the RabGGT polypeptide in vitro, and the activity of the RabGGT is determined. A potential agent is identified as an agent that affects the enzymatic activity of RabGGT, or binding of RabGGT to one or more of Rab, REP, a Rab/REP complex, or other protein.

Computer analysis may be performed with one or more of the computer programs including: O (Jones et al. (1991) Acta Cryst. A47:110); QUANTA, CHARMM, INSIGHT, SYBYL, MACROMODEL; ICM, and CNS (Brunger et al. (1998) Acta Cryst. D54:905). In a further embodiment of this aspect of the invention, an initial drug screening assay is performed using the three-dimensional structure so obtained, preferably along with a docking computer program. Such computer modeling can be performed with one or more Docking programs such as DOC, GRAM and AUTO DOCK. See, for example, Dunbrack et al. (1997) Folding & Design 2:27-42.

It should be understood that in the drug screening and protein modification assays provided herein, a number of iterative cycles of any or all of the steps may be performed to optimize the selection. For example, assays and drug screens that monitor the activity of the RabGGT in the presence and/or absence of a potential modulator (or potential drug) are also included in the present invention and can be employed as the sole assay or drug screen, or more preferably as a single step in a multi-step protocol.

RabGGT structure models and databases of structure information are provided. The structure model may be implemented in hardware or software, or a combination of both. For most purposes, in order to use the structure coordinates generated for the structure, it is necessary to convert them into a three-dimensional shape. This is achieved through the use of commercially available software that is capable of generating three-dimensional graphical representations of molecules or portions thereof from a set of structure coordinates.

In one embodiment of the invention, a machine-readable storage medium is provided, the medium comprising a data storage material encoded with machine readable data which, when using a machine programmed with instructions for using said data, is capable of displaying a graphical three-dimensional representation of any of the structures of this invention that have been described above. Specifically, the computer-readable storage medium is capable of displaying a graphical three-dimensional representation of the RabGGT protein, of a complex of a test agent bound to RabGGT protein, or RabGGT complexed to one or more of a prenyl moiety, a Rab protein, a Rab/REP complex, etc.

Thus, in accordance with the present invention, data providing structural coordinates, alone or in combination with software capable of displaying the resulting three dimensional structure of the enzyme, enzyme complex, and structural elements as described above, portions thereof, and their structurally similar homologues, is stored in a machine-readable storage medium. Such data may be used for a variety of purposes, such as drug discovery, identification of agents that modulate RabGGT activity, but do not substantially modulate FT activity, and the like.

Generally, the invention is implemented in computer programs executing on programmable computers, comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer may be, for example, a personal computer, microcomputer, or workstation of conventional design.

Each program is preferably implemented in a high level procedural or object oriented programming language to communicate with a computer system. However, the programs can be implemented in assembly or machine language, if desired. In any case, the language may be a compiled or interpreted language.

Each such computer program is preferably stored on a storage media or device (e.g., ROM or magnetic diskette) readable by a general or special purpose programmable computer, for configuring and operating the computer when the storage media or device is read by the computer to perform the procedures described herein. The system may also be considered to be implemented as a computer-readable storage medium, configured with a computer program, where the storage medium so configured causes a computer to operate in a specific and predefined manner to perform the functions described herein.

The structure of the RabGGT polypeptide, complexes, and elements thereof, are useful in the design of agents that modulate the activity and/or specificity of the enzyme, which agents may then alter cellular proliferation and/or apoptosis. Agents of interest may comprise mimetics of the structural elements. Alternatively, the agents of interest may be binding agents, for example a structure that directly binds to a region of the RabGGT polypeptide by having a physical shape that provides the appropriate contacts and space filling.

For example, the structure encoded by the data may be computationally evaluated for its ability to associate with chemical entities. This provides insight into an element's ability to associate with chemical entities. Chemical entities that are capable of associating with these domains may alter apoptosis. Such chemical entities are potential drug candidates. Alternatively, the structure encoded by the data may be displayed in a graphical format. This allows visual inspection of the structure, as well as visual inspection of the structure's association with chemical entities.

In one embodiment of the invention, a invention is provided for evaluating the ability of a chemical entity to associate with any of the molecules or molecular complexes set forth above. This method comprises the steps of employing computational means to perform a fitting operation between the chemical entity and the interacting surface of the RabGGT polypeptide; and analyzing the results of the fitting operation to quantify the association. The term “chemical entity”, as used herein, refers to chemical compounds, complexes of at least two chemical compounds, and fragments of such compounds or complexes.

Molecular design techniques are used to design and select chemical entities, including inhibitory compounds, capable of binding to a RabGGT structural or functional element. Such chemical entities may interact directly with certain key features of the structure, as described above. Such chemical entities and compounds may interact with one or more structural functional elements (e.g., binding sites), in whole or in part.

It will be understood by those skilled in the art that not all of the atoms present in a significant contact residue need be present in a binding agent. In fact, it is only those few atoms which shape the loops and actually form important contacts that are likely to be important for activity. Those skilled in the art will be able to identify these important atoms based on the structure model of the invention, which can be constructed using the structural data herein.

The design of compounds that bind to and modulate the activity of a RabGGT polypeptide according to this invention generally involves consideration of two factors. First, the compound must be capable of physically and structurally associating with the domains described above. Non-covalent molecular interactions important in this association include hydrogen bonding, van der Waals interactions, hydrophobic interactions and electrostatic interactions.

Second, the compound must be able to assume a conformation that allows it to associate or compete with a RabGGT structural element. Although certain portions of the compound will not directly participate in these associations, those portions of the may still influence the overall conformation of the molecule. This, in turn, may have a significant impact on potency. Such conformational requirements include the overall three-dimensional structure and orientation of the chemical entity in relation to all or a portion of a binding pocket, or the spacing between functional groups of an entity comprising several interacting chemical moieties.

Computer-based methods of analysis fall into two broad classes: database methods and de novo design methods. In database methods the compound of interest is compared to all compounds present in a database of chemical structures and compounds whose structure is in some way similar to the compound of interest are identified. The structures in the database are based on either experimental data, generated by NMR or x-ray crystallography, or modeled three-dimensional structures based on two-dimensional data. In de novo design methods, models of compounds whose structure is in some way similar to the compound of interest are generated by a computer program using information derived from known structures, e.g. data generated by x-ray crystallography and/or theoretical rules. Such design methods can build a compound having a desired structure in either an atom-by-atom manner or by assembling stored small molecular fragments. Selected fragments or chemical entities may then be positioned in a variety of orientations, or docked, within the interacting surface of the RNA.

Docking may be accomplished using software such as Quanta (Molecular Simulations, San Diego, Calif.) and Sybyl, followed by energy minimization and molecular dynamics with standard molecular mechanics force fields, such as CHARMM and AMBER.

Specialized computer programs may also assist in the process of selecting fragments or chemical entities. These include: GRID (Goodford (1985) J. Med. Chem., 28, pp. 849-857; Oxford University, Oxford, UK; MCSS (Miranker et al. (1991) Proteins: Structure, Function and Genetics, 11, pp. 29-34; Molecular Simulations, San Diego, Calif.); AUTODOCK (Goodsell et al., (1990) Proteins: Structure, Function, and Genetics, 8, pp. 195-202; Scripps Research Institute, La Jolla, Calif.); and DOCK (Kuntz et al. (1982) J. Mol. Biol., 161:269-288; University of California, San Francisco, Calif.)

Once suitable chemical entities or fragments have been selected, they can be assembled into a single compound or complex. Assembly may be preceded by visual inspection of the relationship of the fragments to each other on the three-dimensional image displayed on a computer screen in relation to the structure coordinates. Useful program-s to aid one of skill in the art in connecting the individual chemical entities or fragments include: CAVEAT (Bartlett et al. (1989) In Molecular Recognition in Chemical and Biological Problems”, Special Pub., Royal Chem. Soc., 78, pp. 182-196; University of California, Berkeley, Calif.); 3D Database systems such as MACCS-3D (MDL Information Systems, San Leandro, Calif); and HOOK (available from Molecular Simulations, San Diego, Calif.).

Other molecular modeling techniques may also be employed in accordance with this invention. See, e.g., N. C. Cohen et al., “Molecular Modeling Software and Methods for Medicinal Chemistry, J. Med. Chem., 33, pp. 883-894 (1990). See also, M. A. Navia et al., “The Use of Structural Information in Drug Design”, Current Opinions in Structural Biology, 2, pp. 202-210 (1992).

Once the binding entity has been optimally selected or designed, as described above, substitutions may then be made in some of its atoms or side groups in order to improve or modify its binding properties. Generally, initial substitutions are conservative, i.e., the replacement group will have approximately the same size, shape, hydrophobicity and charge as the original group. It should, of course, be understood that components known in the art to alter conformation should be avoided. Such substituted chemical compounds may then be analyzed for efficiency of fit by the same computer methods described above.

Another approach made possible and enabled by this invention, is the computational-screening of small molecule databases for chemical entities or compounds that can bind in whole, or in part, to the RabGGT polypeptide. In this screening, the quality of fit of such entities to the binding site may be judged either by shape complementarity or by estimated interaction energy. Generally the tighter the fit, the lower the steric hindrances, and the greater the attractive forces, the more potent the potential modulator since these properties are consistent with a tighter binding constant. Furthermore, the more specificity in the design of a potential drug the more likely that the drug will not interact as welt with other proteins. This will minimize potential side effects due to unwanted interactions with other proteins.

Compounds known to bind RabGGT, including those described above, can be systematically modified by computer modeling programs until one or more promising potential analogs are identified. In addition systematic modification of selected analogs can then be systematically modified by computer modeling programs until one or more potential analogs are identified. Alternatively a potential modulator could be obtained by initially screening a random peptide library, for example one produced by recombinant bacteriophage. A peptide selected in this manner would then be systematically modified by computer modeling programs as described above, and then treated analogously to a structural analog.

Once a potential modulator/inhibitor is identified it can be either selected from a library of chemicals as are commercially available from most large chemical companies including Merck, Glaxo Welcome, Bristol Meyers Squib, Monsanto/Searle, Eli Lilly, Novartis and Pharmacia Upjohn, or alternatively the potential modulator may be synthesized de novo. The de novo synthesis of one or even a relatively small group of specific compounds is reasonable in the art of drug design.

The success of both database and de novo methods in identifying compounds with activities similar to the compound of interest depends on the identification of the functionally relevant portion of the compound of interest. For drugs, the functionally relevant portion may be referred to as a pharmacophore, i.e. an arrangement of structural features and functional groups important for biological activity. Not all identified compounds having the desired pharmacophore will act as a modulator of apoptosis. The actual activity can be finally determined only by measuring the activity of the compound in relevant biological assays. However, the methods of the invention are extremely valuable because they can be used to greatly reduce the number of compounds which must be tested to identify an actual inhibitor.

In order to determine the biological activity of a candidate pharmacophore it is preferable to measure biological activity at several concentrations of candidate compound. The activity at a given concentration of candidate compound can be tested in a number of ways.

In some embodiments, the activity of the candidate compound is tested for its activity in modulating RabGGT enzymatic activity. RabGGT enzymatic activity is quantified using a filter binding assay that measures the transfer of (3H) geranylgeranyl groups (GG) from all-trans-(³H)geranylgeranyl pyrophosphate (³H-GGPP) to recombinant Rab3A protein (Shen and Seabra (1996) J. Biol. Chem. 271:3692; Armstrong et al. (1996) Methods in Enzymology 257:30), or as described in the Examples.

In some embodiments, the activity of the candidate compound is tested for its activity in modulating an interaction between RabGGT and a RabGGT interacting protein, as described above. Suitable assays include a yeast two-hybrid assay, a FRET assay, a BRET assay, a fluorescence quenching assay; a fluorescence anisotropy assay; an immunological assay; and an assay involving binding of a detectably labeled protein to an immobilized protein.

In other embodiments, the activity of the candidate compound is tested for its activity in modulating FT enzymatic activity. The enzymatic activity of farnesyl transferase can be measured using any known method, e.g., the method described in Mann et al. (1995) Drug Dev. Res. 34:121, or in Ding et al. (1999) J. Med. Chem. 42:5241.

In other embodiments, the activity of the candidate compound is tested for its activity in increasing or decreasing apoptosis. Assays can be conducted on cell populations or an individual cell, and include morphological assays and biochemical assays. A non-limiting example of a method of determining the level of apoptosis in a cell population is TUNEL (TdT-mediated dUTP nick-end labeling) labeling of the 3′-OH free end of DNA fragments produced during apoptosis (Gavrieli et al. (1992) J. Cell Biol. 119:493).

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s, second(s); min, minute(s); hr, hour(s); and the like.

### Example 1

### Methods for Preparation of Compounds 7A-7T

This example provides methods for synthesis of compounds 7A through 7T.

Compounds 7A, 7B, 7H, 7I, and 7J. (structures shown below) may be prepared by the general procedures described by Ding et al., in U.S. Pat. No. 6,011,029, issued Jan. 4^{th}, 2000. Compounds 7C, 7D, 7N, 7O, 7P, 7Q, 7R, 7S, and 7T (structures shown below) may be prepared by the general procedures described by Bhide et al., in U.S. Pat. No. 6,387,926, issued May ₁₄^{th}, 2002. The contents of U.S. Pat. Nos. 6,011,029, and 6,387,926 are hereby incorporated by reference in their entireties.

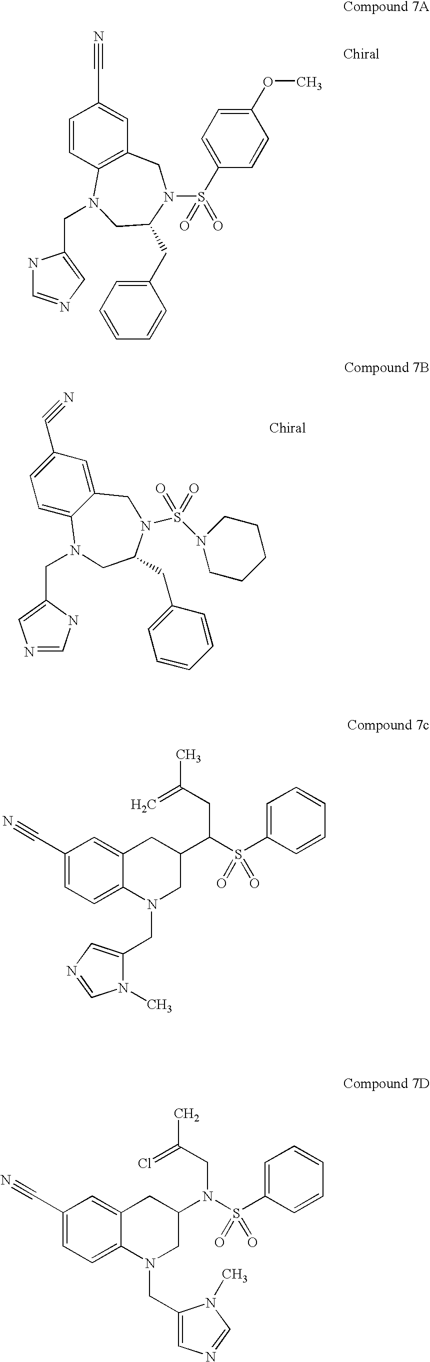

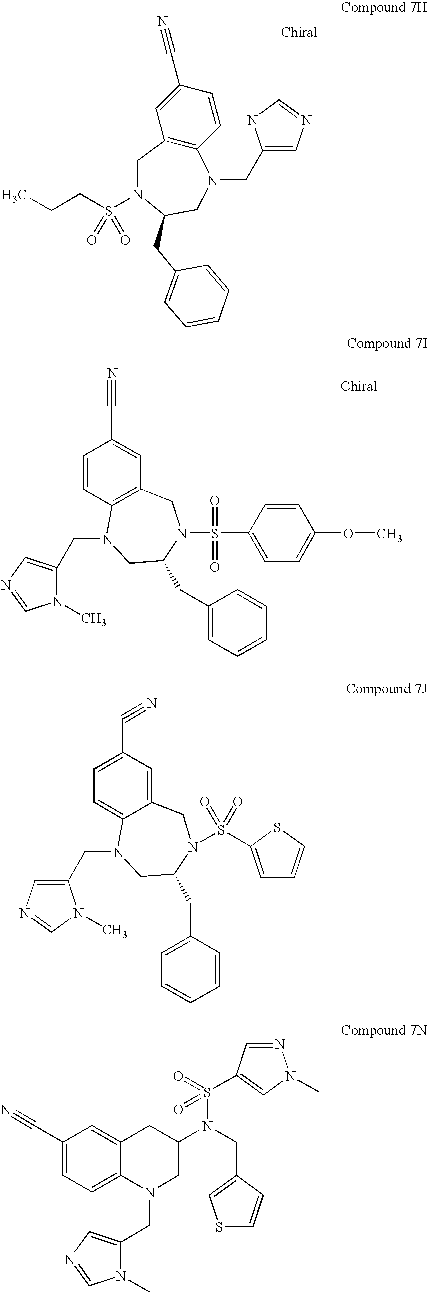

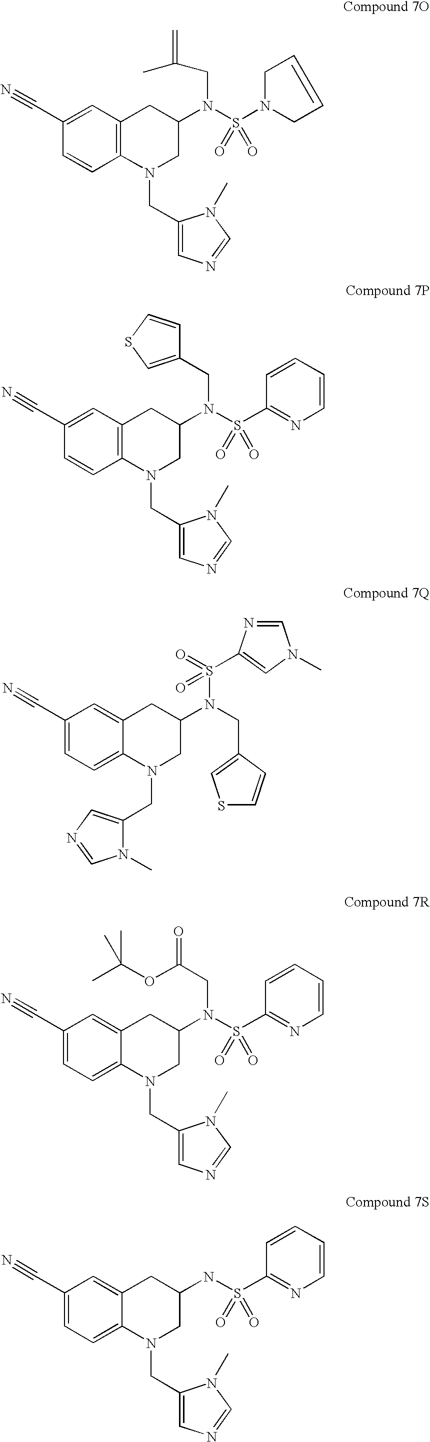

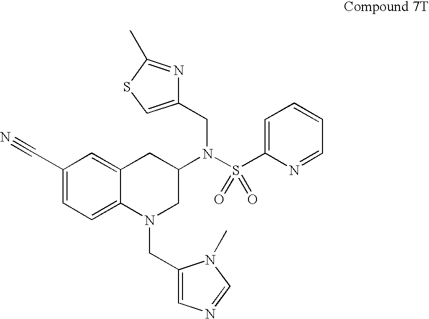

### Example II

### Compound-Induced Apoptosis in HCT-116 Human Colon Tumor Cells

This example demonstrates that a specific apoptotic phenotype can be obtained by treatment of mammalian tissue culture cells with compounds that come from two major structural classes.

Methods

HCT-116 human colon tumor cells obtained from the American Type Culture Collection (ATCC) were grown in McCoy's 5A culture medium with 10% heat inactivated FBS, 1× penicillin/streptomycin, and 25 mM HEPES, in an incubator maintained at 37° C. with CO₂ at 6-7% and humidity at 95%. Cells were treated with compounds using a dose range from 0.04 μM to 100 μM. After 48 hours they were examined by microscopy for signs of cell rounding, vaccuolation, and nuclear condensation. These are morphological markers associated with apoptosis, and are consistent with results obtained by performing an assay for nucleosomal DNA, or a TdT-mediated dUTP nick end labeling (TUNEL) assay.

Results and Conclusions

Results of the apoptosis assay are presented in Table 1. The concentrations cited are the minimal concentration required to induce these morphological changes in 50% of the treated cells. Compounds 7A, 7B, 7D, 7H, 7I, 7J, and 7N induce apoptosis with varying potency: compound 7I is the most potent, with a minimum effective concentration of 40 nM, while 7A, 7D and 7N require treatment at 3.7 μM to produce apoptosis in 50% of cells. Compound 7C and compounds 70 through 7T are very weak effectors of apoptosis, requiring concentrations over 250 times higher than compounds 7B and 7H.

TABLE 1
Induction of apoptosis in HCT116 cells by compounds from
two structural classes

| Compound | Structural class | 50% APOPTOTIC, μM |
|---|---|---|
| 7A | Benzodiazepine | 3.3 |
| 7B | Benzodiazepine | 0.37 |
| 7C | Tetrahydroquinoline | 10 |
| 7D | Tetrahydroquinoline | 3.3 |
| 7H | Benzodiazepine | 0.37 |
| 7I | Benzodiazepine | 0.04 |
| 7J | Benzodiazepine | 2.50 |
| 7N | Tetrahydroquinoline | 3.3 |
| 7O | Tetrahydroquinoline | 10 |
| 7P | Tetrahydroquinoline | 25 |
| 7Q | Tetrahydroquinoline | 30 |
| 7R | Tetrahydroquinoline | 30 |
| 7S | Tetrahydroquinoline | 50 |
| 7T | Tetrahydroquinoline | 90 |

### Example III

### Compound Induced Regression of Tumors In Vivo

This example demonstrates that tumor regression resulting in complete cure was observed in a human tumor xenograft model in which one of the compounds was evaluated.

Methods

Compound 7H was evaluated against a human tumor xenograft model; this data has been presented by Hunt et al. (2000, J. Med. Chem. 43:3587). Fragments of the HCT116 colon tumor were implanted subcutaneously in mice, and allowed to grow. The period of time required for tumor volume to double, TVDT, was determined. Compound administration was initiated when tumors were between 100 and 300 mg. Compound was dissolved in 10% ethanol and dosed orally once daily at 600 mg/kg for ten doses, Monday through Friday. Groups of eight mice were treated. Cures were evaluated after elapse of a post-treatment period that was greater than ten TVDT. A mouse was considered cured when no mass that was larger than 35 mg was present at the site of tumor implant. Drug-treated mice that died before the first death in the parallel control group were considered to have died from drug-related toxicity. Groups of mice with more than one death were not used in the evaluation of efficacy.

Results and Conclusions

Among the eight mice treated with compound 7H, seven mice experienced cure of the tumor, with one death that was attributed to drug related toxicity. The observation that treatment with compound 7H produces tumor regression resulting in complete cure is consistent with a model in which the compound acts on a cellular target to cause death.

### Example IV

### Compound-Induced Apoptosis in the C. elegans Germline

This example demonstrates that treatment with the compounds also produces a specific apoptotic effect on the nematode C. elegans.

Methods

The compounds were applied to early larval and adult C. elegans hermaphrodites by mixing a concentrated DMSO solution of the compound with heat-killed OP50 bacteria in a salt solution. The bacteria were then applied to agar plates and worms of the appropriate age seeded onto the plates. Compounds 7A, 7B, 7C, 7D, 7H, 7I and 7J were applied to worms at a final concentration of 1.5 mM. and the resulting visible phenotypes analyzed. The phenotype of apoptosis in C. elegans was quantified as follows: Germ cells in the C. elegans hermaphrodite gonad progress through various stages of differentiation to become mature ova. At the pachytene stage of meiotic prophase, some germ cells undergo programmed cell death (apoptosis) as part of normal development. The apoptotic corpses resulting from this process can be visualized by high-resolution Nomarski optics and are readily distinguishable cells to the trained eye from viable germ cells by their compact, button-like appearance. Necrotic cells, which are rarer, have a less compact appearance. Apoptosis is most reliably distinguished from necrosis, however, by its requirement for the core apoptotic machinery, such as a functional caspase/ced-3 gene. Since C. elegans has symmetrical anterior and posterior gonad structures, referred to as “arms”, apoptosis is scored by visually counting the apoptotic corpses present in a 1-2 day old adult in each germline arm. Normal, untreated worms rarely contain more than 2 corpses per arm. In a treated sample, the number of worms that contain more than 2 corpses provides a very accurate indicator of the apoptotic effect of the treatment.

Results and Conclusions

Compounds 7A, 7B, 7C, 7D, 7H, 7I and 7J were applied to groups of 10-19 worms, and worms were examined for an apoptosis phenotype in the germline. The results are presented in Table 2. Adult worms treated with compound 7B showed the most striking increase in the number of apoptotic corpses in the adult germline. For example, while a typical germline arm in untreated wild-type adult worms contains 0-2 apoptotic corpses at any time (the average is 0.6 corpses/arm); treatment with compound 7B at 0.8 mM or higher increased the observed number of corpses to 5-7. Compounds 7A, 7C, 7D, 7H, 7I and 7J were found to have a similar effect to compound 7B, increasing the mean number of apoptotic corpses in the germline. In FIG. 1, the percentage of the germline arms from each treated group that contain more than 2 apoptotic corpses is displayed.

TABLE 2
Frequency of observation of the stated number of apoptotic corpses per
germline arm in wild-type worms treated with
either compound or a vehicle control.

| | | | | | | | | | | % arms |
|---|---|---|---|---|---|---|---|---|---|---|
| | Corpses/germline arm | | | | | | N | | | with >2 |
| | 0 | 1 | 2 | 3 | 4 | >4 | tested | mean | SD | corpses |
| Vehicle | 7 | 4 | 0 | 0 | 0 | 0 | 11 | 0.4 | 0.5 | 0 |
| 7A | 1 | 4 | 2 | 3 | 0 | 1 | 11 | 2.0 | 1.4 | 36 |
| 7B | 0 | 0 | 1 | 1 | 0 | 10 | 12 | 6.9 | 2.8 | 92 |
| 7C | 0 | 0 | 0 | 4 | 0 | 6 | 10 | 4.4 | 1.3 | 100 |
| 7D | 0 | 2 | 3 | 2 | 2 | 1 | 10 | 3.0 | 2.1 | 50 |
| 7H | 5 | 4 | A | 3 | 2 | 0 | 17 | 1.6 | 1.4 | 29 |
| 7I | 1 | 3 | 3 | 1 | 2 | 1 | 12 | 2.3 | 1.7 | 33 |
| 7J | 3 | 4 | 1 | 4 | 4 | 3 | 19 | 2.8 | 2.3 | 59 |
| 7K | 5 | 3 | 6 | 3 | 1 | 1 | 19 | 1.7 | 1.4 | 26 |

### Example V

### The Compounds Mediate Apoptosis Via the Canonical Pathway

This example demonstrates that the specific apoptotic effects of the compounds on C. elegans are abolished by a mutation in caspase/ced-3 or in APAF-1/ced-4, indicating that the compounds mediate their effects via the canonical apoptotic pathway.

Methods

Early larval and adult C. elegans hermaphrodites were treated with compound and the phenotype of apoptosis in the germline arm was quantified as described in Example IV.

Results and Conclusions

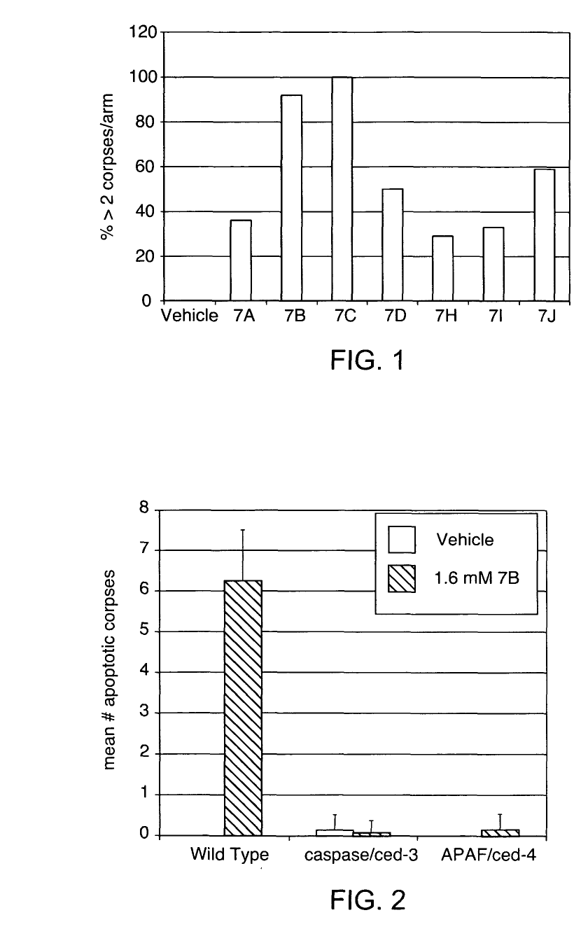

Early larval and adult C. elegans hermaphrodites that were mutant for the genes for caspase/ced-3 or APAF-1/ced-4 were treated with compound 7B at 1.6 mM, and the phenotype of apoptosis in the germline arm was quantified. Table 3 contains the numerical data from this experiment, and FIG. 2 provides a graphical display of the data. While treatment of wild-type worms with compound 7B increases the average number of apoptotic corpses per germline arm from an average of 0.4 per arm to an average of 6.9 per arm, no increase in corpses was observed when caspase/ced-3 or in APAF-1/ced-4 mutants were treated. This observation shows that the drug-induced increase in frequency of germline corpses described in Example IV is dependent on the presence of functional components of the canonical apoptotic pathway, and supports the assertion that the increase in corpses is indeed due to an increase in apoptosis.

TABLE 3
Frequency of observation of the stated number of apoptotic corpses per
germline arm in wild-type or mutant worms treated with 7B or vehicle.

| Geno- | | Corpses/germline arm | | | | | | N | | | % arms with >2 |
|---|---|---|---|---|---|---|---|---|---|---|---|
| type | | 0 | 1 | 2 | 3 | 4 | >4 | tested | mean | SD | corpses |
| WT | Vehicle | 11 | 0 | 0 | 0 | 0 | 0 | 11 | 0 | 0 | 0 |
| | 7B | 0 | 0 | 0 | 1 | 0 | 10 | 11 | 6.25 | 1.25 | 100 |
| ced3 | Vehicle | 11 | 2 | 0 | 0 | 0 | 0 | 13 | 0.15 | 0.38 | 0 |
| | 7B | 12 | 1 | 0 | 0 | 0 | 0 | 13 | 0.08 | 0.28 | 0 |
| ced4 | Vehicle | 10 | 0 | 0 | 0 | 0 | 0 | 10 | 0 | 0 | 0 |
| | 7B | 11 | 2 | 0 | 0 | 0 | 0 | 13 | 0.15 | 0.38 | 0 |

### Example VI

### RNAi of mRNA for RabGGT Subunits Causes Apoptosis in C. elegans

This example demonstrates that treatment of the nematode C. elegans with a reagent that destroys the messenger RNA (RNAi) against either subunit of RabGGT results in a specific apoptotic phenotype.

Methods

DNA encoding GGTase alpha/M57.2 (GenBank entry NM-067966) and GGTase beta/B0280.1 (GenBank entry NM 066158) was amplified from a C. elegans genomic DNA template by PCR (Takara LA Taq DNA polymerase) using oligonucleotides containing gene-specific priming sequences that were flanked by sequences encoding the T7 polymerase priming site. The gene-specific priming sequences targeted the first 5 exons of B0280.1 (product size˜2 kiloBases) and the first four exons of M57.2 (product size˜1 kiloBases). The PCR products were analyzed by gel electrophoresis to confirm that the correct product size was obtained. RNA was transcribed from the PCR product using the MEGAscript High Yield Transcription Kit (Ambion) according to manufacturer's instructions. Directly after transcription, the RNA was annealed by heating to 68° C. for 20 minutes. The double stranded RNA (dsRNA) was checked for product quality by gel electrophoresis. The dsRNA was then ethanol-precipitated, washed once with 100% ethanol and twice with 70% ethanol and the pellet was allowed to air dry for 30 minutes. The dsRNA was re-suspended in 1× IM buffer (20 mM KPO₄, 3 mM potassium citrate, 2% PEG 6000) in volume equal to the original in vitro transcription reaction, and stored at −20° C.

For RNAi treatment of worms, wild type animals at the L2/L3 stage of development were collected in M9 buffer at˜50 animals/μl (M9 is 0.044 M KH₂PO₄, 0.085 M Na₂HPO₄, 0.18 M NaCl and 1 mM MgSO₄). 1 μl of this nematode suspension was added to 3 μl of dsRNA and incubated for 24 hours in a sealed 96 well plate at 20° C. in a humidified chamber.

Animals were allowed to develop to adulthood before compound treatment and/or assay of germline apoptosis as described in Example IV.

Results and Conclusions

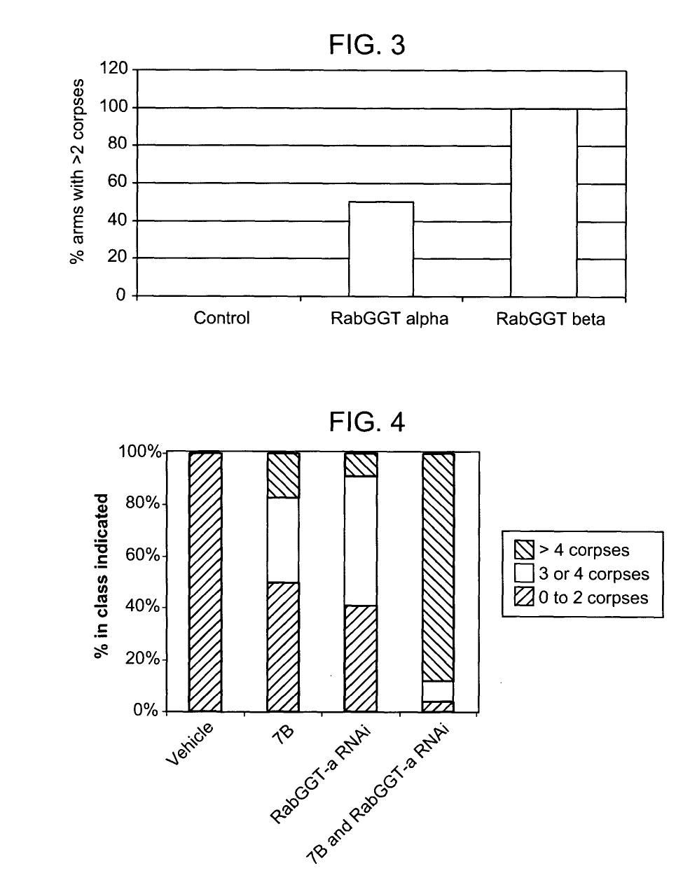

Use of an RNAi reagent against either the alpha or beta subunit of the nematode RabGGT enzyme was found to induce the formation of apoptotic corpses in the germline of C. elegans. While a typical germline arm in untreated adults contains, on average, less than one apoptotic corpse; treatment with an RNAi reagent against the RabGGT alpha subunit increased the average number observed to 2.4 corpses/arm. Treatment with an RNAi reagent against the RabGGT beta subunit increased the average number observed to 9 corpses/arm. The graph displayed in FIG. 3 shows the percentage of germline arms that contained greater than 2 apoptotic corpses. Ablation of the mRNA for a protein by RNAi or other methods has been demonstrated to result in a reduction of the quantity and hence cellular function of the encoded protein. Thus, it appears that a reduction in RabGGT function is sufficient to induce apoptosis in cells of the C. elegans germline.

### Example VII

### Genetic Analysis of Sensitivity Connects the Compound Activity and Rab GGTase in Inducing Apoptosis

This example demonstrates that treatment of the nematode C. elegans with a low dose of RNAi against a RabGGT subunit acts in synergy with low doses of this same set of compounds, to result in a specific apoptotic phenotype.

Methods

Early larval and adult C. elegans hermaphrodites were treated with compound as described in Example IV. RNAi preparation and treatment was performed as described in Example VI. The phenotype of apoptosis in the germline arm was quantified as described in Example IV.

Results and Conclusions

To test the hypothesis that RabGGT is a direct target of the 7B compound, we examined the effect of a low dose of compound 7B (0.3 mM) on the amount of apoptosis induced by a reduction in RabGGT function. The rationale behind the experiment is as follows: the effect of a submaximal compound dose will be substantially increased if the target activity is already partially compromised. Since RNAi directed against the alpha subunit of RabGGT induces a lower level of germline apoptosis than RNAi directed against the beta subunit, RNAi directed against the alpha subunit of RabGGT (RabGGT-alpha RNAi) was used to mimic a partial loss of function of the enzyme in adult worms. Table 4 contains data for each treatment administered separately, and for the treatments administered together. Co-administration of the RabGGT-alpha RNAi reagent with 0.3 mM of compound 7B causes an increase in the level of observed apoptosis which is far greater than the additive value of the independent treatments. This can be seen very clearly when the number of germline arms containing more than four apoptotic corpses is quantified (Table 4) and displayed graphically (FIG. 4). In compound treated worms, 17% of arms have greater than four corpses, while in RNAi treated worms, 9% of arms have greater than four corpses. Co-administration of the RabGGT-alpha RNAi reagent with compound 7B increases the percentage of arms with more than 4 corpses to 88%. Thus, hypersensitivity to the compound is observed when RabGGT activity is compromised. These findings are consistent with a model in which compound 7B induces apoptosis in C. elegans by inhibiting the activity of the RabGGT enzyme.

TABLE 4
Frequency of observation of the stated number of apoptotic corpses per
germline arm in wild-type worms treated with compound 7B
and/or RNAi against the RabGGT alpha subunit.

| | | | | | | | | | | % arms | % arms | % arms |
|---|---|---|---|---|---|---|---|---|---|---|---|---|
| | Corpses/arm | | | | | | N | | | with 0-2 | with 3-4 | with >4 |
| | 0 | 1 | 2 | 3 | 4 | >4 | tested | mean | SD | corpses | corpses | corpses |
| Vehicle | 9 | 10 | 3 | 0 | 0 | 0 | 22 | 0.73 | 0.7 | 100 | 0 | 0 |
| 7B | 5 | 5 | 2 | 5 | 3 | 4 | 24 | 2.3 | 1.8 | 50 | 33 | 17 |
| RNAi | 2 | 3 | 4 | 5 | 6 | 2 | 22 | 2.7 | 1.5 | 41 | 50 | 9 |
| 7B and | 0 | 1 | 0 | 0 | 2 | 21 | 24 | 8.0 | 3.0 | 4 | 8 | 88 |
| RNAi | | | | | | | | | | | | |

### Example VIII

### Genetic Analysis of Resistance Connects the Compound Activity and Rab GGTase in Inducing Apoptosis

This example demonstrates that a mutation in the nematode C. elegans that confers resistance to the apoptotic effects of the compounds also confers resistance to the apoptotic effects of RNAi against a RabGGT subunit.

Methods

Early larval and adult C. elegans hermaphrodites were treated with compound as described in Example IV. RNAi preparation and treatment was performed as described in Example VI. The phenotype of apoptosis in the germline arm was quantified as described in Example IV.

Results and Conclusions

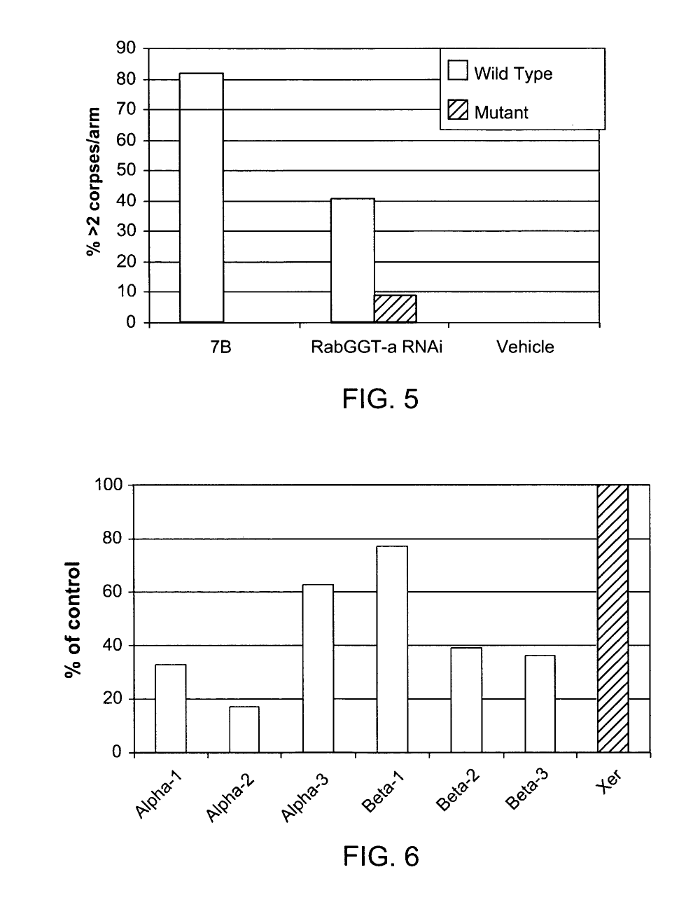

As a further genetic test of the interaction between compound 7B and RabGGT, we examined the effect of a reduction in RabGGT activity in mutants that are resistant to compound 7B. The rationale was as follows: if compound 7B induces apoptosis by inactivation of RabGGT, then the same mutations that decrease 7B-induced apoptosis would be expected to decrease the apoptotic effect induced by lack of RabGGT. We examined a mutant strain that is strongly resistant to induction of apoptosis by compounds 7A-J. The resistance conferred by this mutation appears specific to compounds of the type exemplified by 7A-7J, since the mutant does not display any cross-resistance to the effects of a range of unrelated compounds (data not shown). RNAi treatment against the RabGGT alpha subunit was performed on this strain as described in Example VI. In the mutant strain the apoptotic effect of RNAi treatment against the RabGGT alpha subunit was strongly reduced (FIG. 5). Thus we have shown that a mutant that is resistant to compound 7B-induced apoptosis is also insensitive to RabGGT (RNAi)-induced apoptosis. These findings are consistent with the model that compound 7B induces apoptosis in C. elegans by inactivating the RabGGT enzyme.

### Example IX

### RNAi of mRNA for RabGGT Subunits Inhibits Proliferation in a Human Cell Line

This example demonstrates that RNAi treatment of a human cell line with reagents against either the alpha or the beta subunit of the RabGGT enzyme has an anti-proliferative effect.

Methods

HCT-116 human colon tumor cells obtained from the ATCC were grown in RPMI culture medium supplemented with 10% heat inactivated FBS, 1× penicillin/streptomycin, and 25 mM HEPES, in an incubator maintained at 37° C. with CO₂ at 6% and humidity at 95%. HCT116 cells were plated in 96 well plates at 2000 cells/100 μl media per well and incubated for 24 hours before RNAi treatment. For treatment, a 2× solution of Lipofectamine 2000/siRNA complexes was generated for each individual siRNA as follows. The siRNA oligonucleotides (Xeragon; Huntsville Ala.) were diluted to a final concentration of 1 μM in Optimem serum-free media (Invitrogen; Carlsbad, Calif.) and incubated for 5 minutes at room temperature. The Lipofectamine 2000 reagent (Invitrogen; Carlsbad, Calif.) was diluted to 10 μg/ml in Optimem serum-free media and incubated for 5 minutes at room temperature. Equal volumes of the 1 μM siRNA oligonucleotides and the 10 μg/ml Lipofectamine 2000 were mixed together, giving a 5× stock of siRNA/Lipofectamine 2000 complexes. After incubation for 20 minutes at room temperature, 1.5 volumes of RPMI medium containing 10% heat inactivated FBS was added to the 5× stock, resulting in a 2× stock of siRNA/Lipofectamine 2000 complexes. For RNAi treatment, 100 μof the 2× stock of siRNA/Lipofectamine 2000 complexes was added to each well containing HCT116 cells, to give a final concentration of 1× siRNA/Lipofectamine 2000 complexes. Cells were incubated for 72 hours prior to the proliferation assay. Three replicates were performed for each siRNA treatment.

The effect of RNAi treatment directed against RabGGT subunits on cellular proliferation was assayed using a 3H-thymidine incorporation assay. The principle of this assay is as follows: During S-phase of the cell cycle, cells incorporate thymidine into the new strand of genomic DNA. Tritiated thymidine can be added to the culture medium and will be incorporated into genomic DNA in proportion to the number of rounds of DNA synthesis that occur. Incorporation can be quantified following lysis of the cells and removal of unincorporated nucleotides. RNAi-treated cells prepared as described above were assayed for 3H-thymidine uptake as follows. The cells were pulsed with 3H-thymidine by addition of 20 μl of a 44 μCi/ml solution of 3H-thymidine in RPMI to each well, to obtain a final concentration of 3H-thymidine of 4 μCi/ml. After incubation for 3 h at 37° C., the medium was removed and 50 μl of 0.25% trypsin in phosphate buffered saline (140 mM NaCl, 2.7 mM KCl, 10 mM Na₂HPO₄ and 1.8 mM KH₂PO₄, pH 7.4) was added. After 10 minutes, the contents of the wells were harvested onto a 96-well GF/C filter plate (Whatman; Clifton N.J.) using a Hewlett Packard Filtermate. The filter plate was washed 10 times with distilled water, then left to dry overnight. After the addition of 50 μl of Microscint-20 scintillation fluid (Perkin Elmer; Boston, Mass.) per well, the filter plates were sealed and the amount of radioactivity retained on the filter was determined by scintillation counting. The average of the three replicate samples is reported.

### Results and Conclusions

We designed synthetic double-stranded oligonucleotides (siRNAs) suitable for performing RNAi treatment against either the alpha subunit (Genbank entry NM_{—}004581) or beta subunit (Genbank entry NM_{—}004582) of the human RabGGT enzyme (Table 5). Treatment of the HCT 116 human colon cell line with siRNA reagents against the alpha subunit resulted in a reduction of 3H-thymidine incorporation that ranged from 17% to 63% of control values (Table 5). Treatment of the HCT 116 human colon cell line with siRNA reagents against the beta subunit resulted in a reduction of 3H-thmidine incorporation that ranged from 36% to 77% of control values (Table 5). Thus, RNAi treatment with all six of the siRNA reagents against RabGGT resulted in a reduction in 3H-thymidine uptake. This result is displayed graphically in FIG. 6. Varying efficacy among siRNAs targeting the same gene is not uncommon, since the characteristics that are required for effective destruction of the target mRNA are not understood (Elbashir et al., 2002; Methods 26:199). The observed reduction in 3H-thymidine incorporation resulting from RNAi treatment against RabGGT could be the result of an inhibition of proliferation, or the result of increased cell death among the treated cells. This data is consistent with a model in which a reduction in function of the RabGGT enzyme results in apoptosis.

TABLE 5

Structure of siRNA reagents and
effect on 3H-thymidine incorporation in HCT116 cells

| | | | | Bases of | 3H-thy |
|---|---|---|---|---|---|
| | | siRNA sense | siRNA antisense | coding region | incorp. % |
| siRNA | Gene targeted | strand | strand | targeted | of control |
| Alpha-1 | RabGGT-alpha | GGCAGAACU | CAGGAAGCC | 268-291 | 33 |
| | | GGGCUUCCU | CAGUUCUGC | | |
| | | GTT (SEQ ID | CTT (SEQ ID | | |
| | | NO:01) | NO:02) | | |
| Alpha-2 | RabGGT-alpha | AGAGCUGGA | CUGCACCAGC | 628-651 | 17 |
| | | GCUGGUGCA | UCCAGCUCUT | | |
| | | GTT (SEQ ID | T (SEQ ID | | |
| | | NO:03) | NO:04) | | |
| Alpha-3 | RabGGT-alpha | GAUGGAGUA | CACCUCGGCA | 1309-1332 | 63 |
| | | UGCCGAGGU | UACUCCAUCT | | |
| | | GTT (SEQ ID | T (SEQ ID | | |
| | | NO:05) | NO:06) | | |
| Beta-1 | RabGGT-beta | CUUUGGCUU | UUCCCCAACA | 493-516 | 77 |
| | | UGUUGGGGA | AAGCCAAAGT | | |
| | | ATT (SEQ ID | T (SEQ ID | | |
| | | NO:07) | NO:08) | | |
| Beta-2 | RabGGT-beta | CGACAAUUA | CGCCUGAGG | 662-685 | 39 |
| | | CCCUCAGGCG | GUAAUUGUC | | |
| | | TT (SEQ ID | GTT (SEQ ID | | |
| | | NO:09) | NO:10) | | |
| Beta-3 | RabGGT-beta | GAUGAAGAA | AUCCCCCCGU | 812-835 | 36 |
| | | ACGGGGGGA | UUCUUCAUCT | | |
| | | UTT (SEQ ID | T (SEQ ID | | |
| | | NO:11) | NO:12) | | |
| Non- | none | UUCUCCGAA | ACGUGACAC | none | 100 |
| silencing | | CGUGUCACG | GUUCGGAGA | | |
| | | UTT (SEQ ID | ATT (SEQ ID | | |
| | | NO:13) | NO:14) | | |

### Example X

### Biochemical Assay of Compound Inhibition of RabGGT Activity In Vitro

This example demonstrates that certain compounds inhibit RabGGT activity with nanomolar potency using a direct in vitro assay, and that different structural classes of compound may differ in the dose-response relationship for inhibition.

Methods

The effect of compounds 7A through 7T on RabGGT activity was quantified using a filter binding assay that measures the transfer of (3H) geranylgeranyl groups (GG) from all-trans-(3H)geranylgeranyl pyrophosphate (3H-GGPP) to recombinant Rab3A protein (Shen & Seabra, 1996, JBC, 271 :3692; Armstrong et al., 1996, Methods in Enzymology 257:30). Modifications to published protocols are noted explicitly below.

Recombinant rat RabGGT, expressed using the Sf9/baculovirus system, was purchased from Calbiochem (cat. no. 345855). Recombinant unprenylated human Rab3A was obtained from Panvera.(cat. no. P2173). Human REβ-1, expressed in Sf9 cells, was obtained from Calbiochem (cat. no. 554000). Tritium labeled geranylgeranyl pyrophosphate was purchased from Amersham Pharmacia Biotech (15 Ci/mmol). Unlabeled GGPP was purchased from Sigma (cat. no. G-6025).

The reaction buffer contained 50 mM HEPES pH7.4, 5 mM MgCl₂, 1 mM DTT, 1 mM Nβ-40. Solutions of RabGGT, Rab3A, REP-1, and GGPP were prepared in this reaction buffer. Final protein concentrations in the reaction mixture were modified from the published protocols, with the standard reaction mixture containing 2 μM Rab3A, 0.2 μM REP-1, 5 μM unlabeled GGPP, 0.5 μM labeled GGPP, and 10-50 nM RabGGT in a total volume of 20 μl. The specific activity of (3H)GGPP used in the assay was 3000 dpm/pmol.

Compounds were prepared as 50 mM stocks in DMSO and diluted to give an appropriate concentration for the assay as a 20% DMSO stock. 2 μl of the diluted compound stock was added to a 20 μl reaction to give a final DMSO concentration of 2% in the assay.

The order of addition of reagents was altered from the published protocols. Reaction mixtures were prepared by sequentially adding Rab3A and REP-1 proteins to the reaction buffer, followed by compound and RabGGT enzyme to a volume of 18 μl. Reactions were initiated by the addition of 2 μl of a solution that contained unlabeled and labeled GGPP. After a 30 minute incubation at 37° C., 1 ml of stop solution (1 volume of concentrated HCl acid with 9 volumes of ethanol) was added and mixed. The solution was then incubated at room temperature for 1 hour to completely precipitate proteins.

The precipitate was collected by vacuum filtration using a vacuum filtration manifold (Millipore model 1225) onto 25 mm GF/A filters (Whatman) that were prewetted with ethanol. The tubes were rinsed twice with 1 ml ethanol which was also poured over the filters. Each filter was subsequently washed three times with 2 mls of ethanol per wash, dried under vacuum, and then put in scintillation vials. Four milliliters of scintillation fluid was added and the radioactivity was quantified on a scintillation counter. Several types of blank reactions were conducted including withholding the enzyme, the substrate, or the accessory protein REP-1, or replacing the compound solution with a 20% DMSO solution. For the substrate titration experiment, the equimolar amounts of Rab3A and REP-1 were mixed and preincubated for 30 min at room temperature before addition of the enzyme.

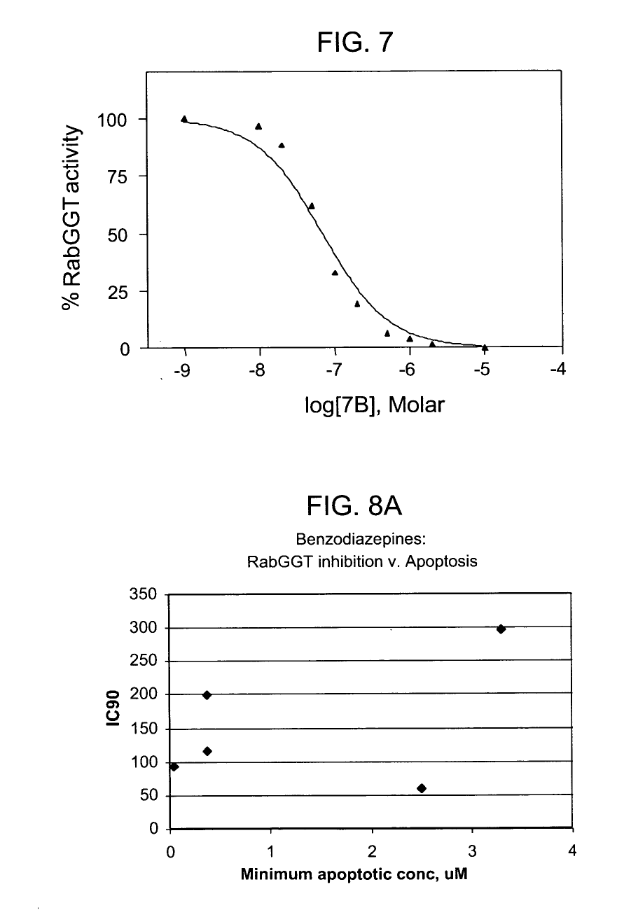

The data was analyzed by non-linear regression analysis methods using the program PRIZM (GraphPad Software, Inc.). Inhibition constants were obtained by analyzing the data using the one site competition equation provided by the software. FIG. 7 presents a typical data series obtained for compound 7B using these methods.

Results and Conclusions

Data presented in Table 6 shows that compounds 7A, 7B, 7H, 7I, 7J, 7N, 7O, 7P, 7Q, and 7S inhibit the activity of rat RabGGT enzyme with IC50 values of less than 100 nM, while 7R and 7T are weaker inhibitors. IC90 values for inhibition of RabGGT are also presented in Table 6. The multiple of the IC90 value relative to the IC90 value is also presented in Table 6. For the benzodiazepine compounds 7A, 7B, 7H, 7I, and 7J, the IC90 value is between 5 and 9 times the IC50 value. For the tetrahydroquinoline compounds 7N, 7O, 7P, 7Q, 7R, 7S and 7T the IC90 value is between 12 and 49 times the IC50 value. The difference in the multiple of the IC90 value relative to the IC90 value for the two classes of compounds indicates that the dose-response relationship is different for each class. Such a difference in dose response may have consequences in an in vivo situation. If it is necessary to completely eliminate the function of an enzyme to produce a given measured effect, IC90 values for inhibition of that enzyme will show a closer relationship to that effect than IC50 values.

TABLE 6
Results of an in vitro assay that measures RabGGT activity
in the presence of compounds.

| | | RabGGT | RabGGT | |
|---|---|---|---|---|
| Compound | Structural class | IC50, nM | IC90, nM | IC90/IC50 |
| 7A | Benzodiazepine | 36 | 295 | 8 |
| 7B | Benzodiazepine | 21 | 199 | 9 |
| 7H | Benzodiazepine | 21 | 115 | 5 |
| 7I | Benzodiazepine | 16 | 93 | 6 |
| 7J | Benzodiazepine | 12 | 58 | 5 |
| 7N | Tetrahydroquinoline | 25 | 309 | 12 |
| 7O | Tetrahydroquinoline | 58 | 1117 | 19 |
| 7P | Tetrahydroquinoline | 84 | 2162 | 26 |
| 7Q | Tetrahydroquinoline | 47 | 2298 | 49 |
| 7R | Tetrahydroquinoline | 541 | 10064 | 19 |
| 7S | Tetrahydroquinoline | 73 | 1404 | 19 |
| 7T | Tetrahydroquinoline | 1433 | >15000 | >10 |

### Example XI

### Relationship Between Inhibition of RabGGT In Vitro and Induction of Apoptosis In Vivo

This example demonstrates a relationship between the level of inhibition of RabGGT enzyme activity in vitro and the ability of the compound to induce apoptosis in an HCTI 16 cell line.

Methods

The assay for compound inhibition of RabGGT function is described in Example X.

Methods for assaying apoptotic activity of compounds on HCTI 16 cells are described in Example II.

Results and Conclusions.

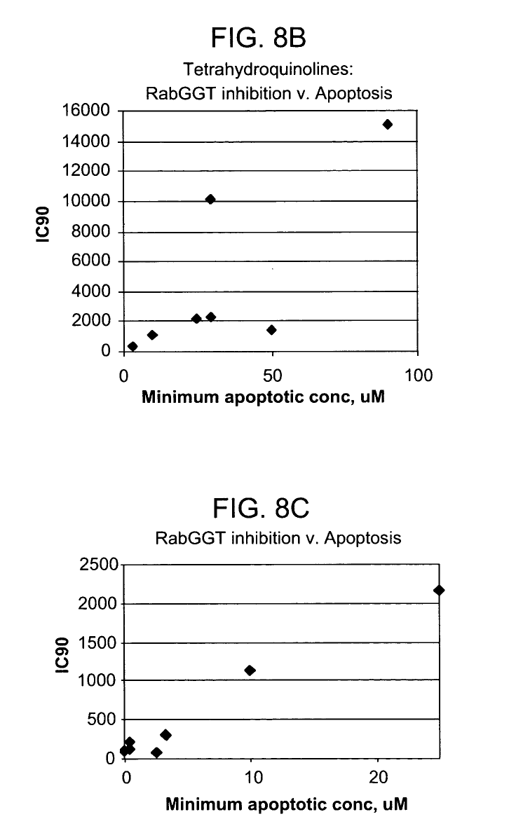

Table 7 provides the IC50 and IC90 values established by biochemical assays for inhibition of RabGGT, and also provides the minimum concentration required to achieve apoptosis of 50% of the HCT116 cells in a culture system. The data for IC90 values and apoptosis values are also presented in a graphical form in FIGS. 8a, 8b, and 8c. In Table 7, compounds are ranked according to their potency in the apoptosis assay and are presented according to structural class.

When IC90 values for RabGGT inhibition are examined, a correlation between potency in the RabGGT inhibition assay and potency in the apoptosis assay is apparent. The square of the Pearson product moment correlation coefficient (the R-squared value) for the apoptosis values and the RabGGT IC90 values is 0.7, which can be interpreted as 70% of the variance in apoptosis values being attributable to the variance in RabGGT inhibition. Of the 12 compounds assayed, only two compounds deviate from their rank order position in Table 7: Compounds 7J and 7S show lower potency in the apoptosis assay than would be predicted by their potency in the RabGGT inhibition assay. Such occasional deviation (2 compounds out of 12) between rank in one assay and rank in another is not unexpected given the number of variables in each assay. We conclude that inhibition of RabGGT activity is related to the apoptotic activity of these compounds.

A correlation between potency in the RabGGT inhibition assay and potency in the apoptosis assay is also apparent when IC50 values for RabGGT inhibition are examined for their relationship to potency in the apoptosis assay. The R-squared value for the apoptosis values and the RabGGT IC90 values is 0.7, which can be interpreted as 70% of the variance in apoptosis values being attributable to the variance in RabGGT inhibition. Compounds 7J, 7P and 7Q deviate from their rank order position. However we note that the tetrahydroquinoline class in general is less potent at inducing apoptosis than would be predicted based on their IC50 value as a measure of potency in the RabGGT inhibition assay. For example, compounds 7A and 7Q have similar IC50 values for RabGGT inhibition, whereas they show a 9-fold difference in potency in the apoptosis assay. The difference in potency in the apoptosis assay is in closer agreement with IC90 values for RabGGT inhibition by 7A and 7Q, which show an 8-fold difference. The observation that IC90 values for RabGGT inhibition show a better relationship to potency in the apoptosis assay than do IC50 values indicates that an almost total loss of cellular RabGGT activity may be required for induction of apoptosis. RabGGT cellular activity may be present in an amount that exceeds the general need, and a cell may be able to subsist with only 50% of that activity present.

TABLE 7
Results of an in vitro assay upon RabGGT activity and
results of an assay of apoptotic activity upon human cells.

| | | HCT116 | | |
|---|---|---|---|---|
| | | 50% | | |
| | | apoptosis, | RabGGT | RabGGT |
| Compound | Structural class | μM | IC50, nM | IC90, nM |
| 7I | Benzodiazepine | 0.04 | 16 | 93 |
| 7H | Benzodiazepine | 0.37 | 21 | 115 |
| 7B | Benzodiazepine | 0.37 | 21 | 199 |
| 7J | Benzodiazepine | 2.5 | 12 | 58 |
| 7A | Benzodiazepine | 3.3 | 36 | 295 |
| 7N | Tetrahydroquinoline | 3.3 | 25 | 309 |
| 7O | Tetrahydroquinoline | 10 | 58 | 1117 |
| 7P | Tetrahydroquinoline | 25 | 84 | 2162 |
| 7Q | Tetrahydroquinoline | 30 | 47 | 2298 |
| 7R | Tetrahydroquinoline | 30 | 541 | 10064 |
| 7S | Tetrahydroquinoline | 50 | 73 | 1404 |
| 7T | Tetrahydroquinoline | 90 | 1433 | >15000 |

In FIG. 8a, Data from the benzodiazepine class of compounds: The IC90 for RabGGT inhibition in nanomoles is shown on the Y axis and the minimum concentration required for induce 50% apoptosis in an HCT116 cell culture is shown on the X axis.

In FIG. 8b, Data from the tetrahydroquinolone class of compounds: The IC90 for RabGGT inhibition in nanomoles is shown on the Y axis and the minimum concentration required for induce 50% apoptosis in an HCT 116 cell culture is shown on the X axis.

In FIG. 8c, Data from compounds 7A through 7Q. Compounds 7R, 7S, and 7T are represented in FIG. 8b, and have been omitted from this figure for graphical clarity rather than because they alter the trend of the observations. The IC90 for RabGGT inhibition in nanomoles is shown on the Y axis and the minimum concentration required for induce 50% apoptosis in an HCT 116 cell culture is shown on the X axis.

### Example XII

### Lack of Relationship Between Inhibition of Farnesyl Transferase (FT) In Vitro and Induction of Apoptosis In Vivo

This example demonstrates that there is no obvious relationship between the level of inhibition of FT enzyme activity in vitro and the ability of the compound to induce apoptosis in an HCT116 cell line.

Methods

Biochemical assays for inhibition of FT were performed as described by Mann et al. (1995, Drug Dev. Res. 34: 121) with the modifications described by Ding et al. (1999, J. Med. Chem., 42:5241)

Methods for assaying apoptotic activity of compounds on HCT116 cells are described in Example II.

Results and Conclusions

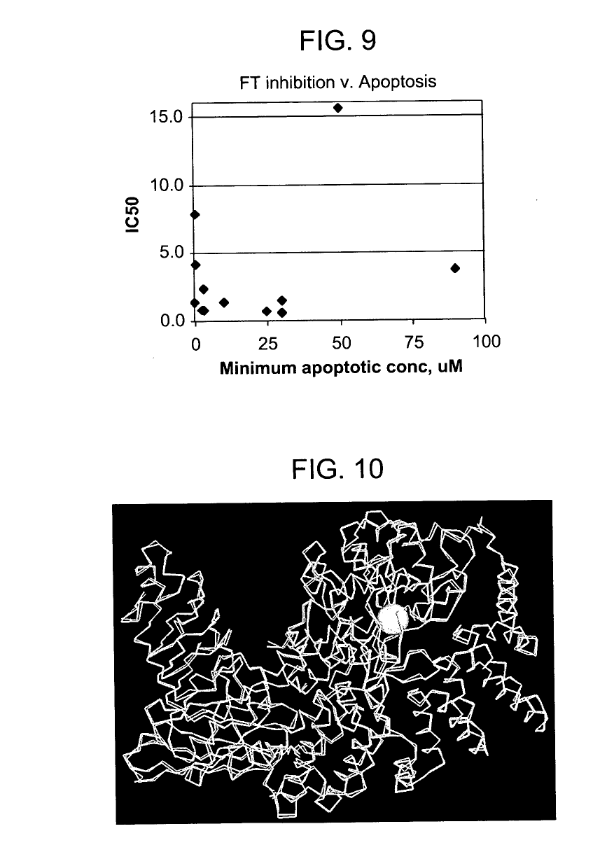

Compounds 7A-7J are from a class of compounds that is predicted to have FT-inhibitory activity (Ding et al., 1999, J. Med. Chem., 42:5241), while compounds 7N-7T also possess structural characteristics that make them potential FT inhibitors. We examined the possibility that inhibition of FT activity was related to the apoptotic activity of these compounds. Table 8 presents the compounds grouped according to structural class and provides the IC50 and IC90 values for inhibition of FT. Table 8 also provides the minimum concentration required to achieve apoptosis of 50% of the HCT116 cells in a culture system. The data for IC50 values and apoptosis values are also presented in a graphical form in FIG. 9.

TABLE 8
Results of an in vitro assay upon FT activity and results of
an assay of apoptotic activity upon human cells.

| | | HCT116 | | |
|---|---|---|---|---|
| | | 50% | | |
| | | apoptosis, | FT | FT |
| Compound | Structural class | μM | IC50, nM | IC90, nM |
| 7I | Benzodiazepine | 0.04 | 1.4 | 11 |
| 7H | Beazodiazepine | 0.37 | 4.1 | 360 |
| 7B | Benzodiazepine | 0.37 | 7.8 | 110 |
| 7J | Benzodiazepine | 2.5 | 0.8 | 7 |
| 7A | Benzodiazepine | 3.3 | 2.4 | 30 |
| 7N | Tetrahydroquinoline | 3.3 | 0.7 | 9 |
| 7O | Tetrahydroquinoline | 10 | 1.4 | 8 |
| 7P | Tetrahydroquinoline | 25 | 0.7 | 4 |
| 7Q | Tetrahydroquinoline | 30 | 0.6 | 6 |
| 7R | Tetrahydroquinoline | 30 | 1.5 | 9 |
| 7S | Tetrahydroquinoline | 50 | 15.5 | 255 |
| 7T | Tetrahydroquinoline | 90 | 3.7 | 48 |

In the data presented in Table 8, compounds are ranked according to their potency in the apoptosis assay. The compounds are all potent inhibitors of FT, with only a 20-fold range being observed in the IC50 values (0.7 nM to 15.5 nM) whereas values in the apoptosis assay range over 2200-fold. When IC50 values for FT inhibition are examined for their relationship to potency in the apoptosis assay, no correlation is apparent. The R-squared value for the apoptosis values and the FT IC50 values is less than 0.1, which can be interpreted as less than 10% of the variance in apoptosis values being attributable to the variance in inhibition of 50% of FT activity. No general correlation with rank order position can be seen; at least 8 compounds deviate between ranking their potency for FT inhibition and ranking their potency for apoptosis induction. The conclusion that there is no correlation between potency in the apoptosis assay and potency in the FT inhibition assay is not altered by examination of IC90 values for FT inhibition. The R-squared value for the apoptosis values and the FT IC90 values is less than 0.01, indicating that none of the variance in apoptosis values is attributable to the variance in inhibiting 90% of FT activity.

FIG. 9 provides a graphical display of the data from Table 8. No trend can be observed in the data by visual inspection. We conclude that inhibition of FT activity is not related to the apoptotic activity of these compounds.

### Example XIII

### Conservation of Structure Between the RabGGT Enzymes from C. elegans, R. norvegicus and H. sapiens

This example demonstrates that the active site of the RabGGT enzyme is conserved between C. elegans, R. norvegicus and H. sapiens, such that a compound which blocks the active site in one species would be reasonably expected to show the same activity in all species.

Methods

Structural models of the RabGGT alpha subunits from C. elegans (GenBank entry NM_{—}067966) and from Homo sapiens (GenBank entry NM_{—}004581) were developed based on sequence alignment with the homologous protein rat RabGGT alpha (GenBank entry NM_{—}031654) whose structure in the RabGGT complex is available in the Protein Data Bank as 1DCE (Zhang et al., 2000, Structure 8:241). Sequence alignments of the RabGGT alpha subunit are shown in Table 9a and Table 10a.

Structural models of the RabGGT beta subunits from C. elegans (GenBank entry NM_{—}066158) and from H. sapiens (GenBank entry NM-004582) were developed based on sequence alignment with the homologous protein rat RabGGT beta (GenBank entry NM_{—}138708) whose structure in the RabGGT complex is available in the Protein Data Bank as 1DCE (Zhang et al., 2000, Structure 8:241). Sequence alignments of the RabGGT beta subunit are shown in Table 9b and Table 10b.

The program LOOK was used for alignments and the model building module within LOOK, SEGMOD, was used to build the homology models (Levitt, (1992), J. Mol. Biol. 226: 507-533; Levitt, (1983), J. Mol. Biol. 170: 723-764). The co-ordinates for the structural model of H. sapiens RabGGT are presented in Table 11 (RabGGT alpha subunit) and Table 12 (RabGGT beta subunit). In both Tables 11 and 12, “Atom No” refers to the atom number within the RabGGT alpha or beta subunit homology model; “Atom name” refers to the element whose coordinates are measured, the first letter in the column defines the element; “Residue” refers to the amino acid within which the atom resides, with the number representing the amino acid number of the “residue”; “X Coord”, “Y Coord”, and “Z Coord” structurally define the atomic position of the element measured in three dimensions.

The quality of the models was evaluated as follows: In order to recognize errors in three-dimensional structures knowledge based mean fields can be used to judge the quality of protein folds (Hendlich et al., 1990, J. Mol. Biol. 216:167). These methods can be used to recognize misfolded structures as well as faulty parts of structural models. The technique generates an energy graph where the energy distribution for a given protein fold is displayed on the y-axis and residue position in the protein fold is displayed on the x-axis. The knowledge based mean fields compose a force field derived from a set of globular protein structures taken as a subset from the Protein Data Bank (Bernstein et al., 1977, J. Mol. Biol. 112:535). An energy value of less than zero is considered to represent a stable 3-dimensional structure. To analyze the quality of a model, the energy distribution of residues is plotted and compared to the energy distribution of the template from which the model was generated.

Results and Conclusions

The amino acid sequence of the H. sapiens RabGGT alpha subunit (HsA) has 91% identity and 93% similarity with that of Rattus norvegicus (RatA). The proteins are both 567 amino acids in length. The amino acid sequence of the H. sapiens RabGGT beta subunit (HsB) has 95% identity and 97% similarity with that of R. norvegicus (RatB). The proteins are both 331 amino acids in length. The crystal structure of a RabGGT complex consisting of the rat alpha and beta subunits has been described at 2 angstrom (A) resolution (H Zhang et al., 2000, Struct. Fold. Des. 8:241). The sequences of HsA and HsB were overlaid onto the crystal structure of the RatA/RatB complex (FIG. 10). There were no insertions or deletions. The free energy plots for the models are shown in FIG. 11. There is near identity between the energy distribution of the model and that of the template from which the model was generated, with the majority of residues having energy values below zero. This indicates that the human RabGGT as modeled represents a stable 3-dimensional structure of high quality.

The putative binding pocket for inhibitors of RabGGT activity can be hypothesized by comparison with farnesyl transferase (FT), a closely related enzyme that has very similar structure and function (Long et al., 2002, Nature 419:645). The structure of FT in complex with known inhibitory compounds has been determined; in this example we used an overlay of an FT/inhibitor complex described by Long et al. (2001, Proc. Natl. Acad. Sci. USA, 98:12948). Of the residues lining the putative binding pocket, all three within the alpha subunit and all 12 within the beta subunit are identical between the two proteins and exist within a region of high conservation and high identity (Table 9a and b). In the enzyme from R. norvegicus, and the enzyme from H. sapiens, the residues within 5A of the active site are Asn A103, Lys A105, Tyr A107, Ser B42, Tyr B44, Leu B45, Trp B52, Arg B144, Asp B238, Cys B240, Tyr B241, Asp B280, Asp B287, Phe B289, His B290, where A refers to the alpha subunit and B to the beta subunit.

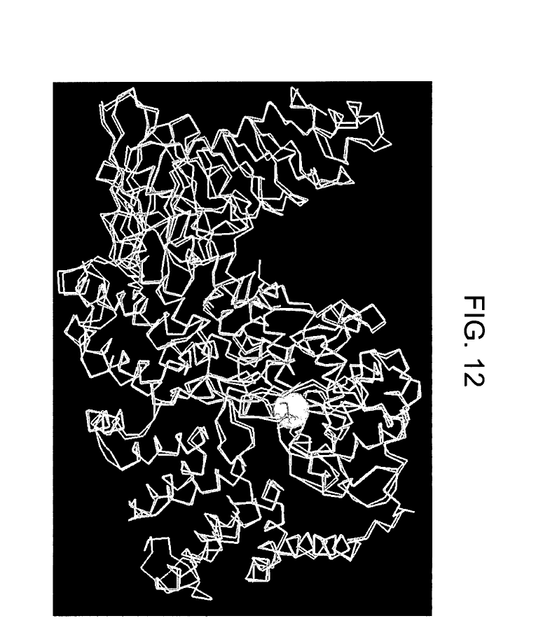

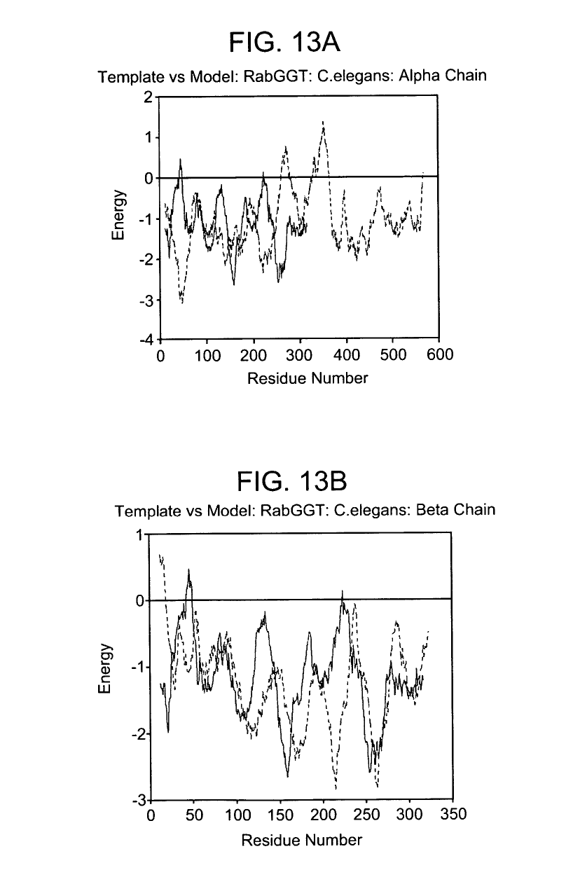

The amino acid sequence of the C. elegans RabGGT alpha subunit (CeA) has 38% identity and 53% similarity with that of R. norvegicus (RatA). RatA is 567 amino acids in length and CeA is 580 amino acids. The amino acid sequence of the C. elegans RabGGT beta subunit (CeB) has 53% identity and 72% similarity with that of R. norvegicus (RatB). RatB is 331 amino acids in length and CeB is 335 amino acids. The sequences of CeA and CeB were overlaid onto the crystal structure of the RatA/RatB complex (FIG. 12). One large insertion in CeA (80-94) corresponded to a loop between helices 3 and 4 in RatA. A substantial deletion in CeA at residue 316, corresponding to RatA residues 300-305, occurs within a beta-sheet at some distance from the proposed binding site and near a large loop. Another insertion in CeA (residues 439-442 at RatA 428) is also at some distance from the binding site and appears to occur with helix 17 of the RatA structure. The free energy plots for the models are shown in FIG. 13. There is a strong correspondence between the energy distribution of the model and that of the template from which the model was generated, with the majority of residues having energy values below zero. This indicates that the C. elegans RabGGT as modeled represents a stable 3-dimensional structure of high quality.

Of the residues lining the putative binding pocket of RabGGT, all three residues within the alpha subunit are identical between the two proteins and exist within a region of high conservation and high identity. Of the 12 residues in the beta subunit determined to be in the binding pocket, all but two were identical and existed in regions of high identity (Table 9a and 9b). In the enzyme from C. elegans, the residues within 5A of the active site are Asn A119, Lys A121, Tyr A123, Ala B48 (non-identity to rat), His B50 (non-identity to rat), Leu B51, Trp B58, Arg B150, Asp B244, Cys B246, Tyr B247, Asp B286, Asp 293, Phe B295, His B296, where A refers to the alpha subunit and B to the beta subunit.

The data presented in this example demonstrates that high quality structural models of human and nematode RabGGT structure can be generated based on the crystal structure that has been obtained for the rat protein. In these models, the active site of the RabGGT enzyme is conserved between C. elegans, R. norvegicus and H. sapiens, such that a compound which blocks the active site in one species would be reasonably expected to show the same activity in all species. Therefore the observation that certain compounds inhibit the rat RabGGT enzyme with nanomolar potency (data presented in Example X), indicates that these compounds would have the same inhibitory effect when applied to the human RabGGT enzyme. The apoptotic effect of the same compounds when applied to C. elegans (data presented in Example IV) may also be interpreted as arising from inhibition of RabGGT, given that the active site of the nematode enzyme is conserved with respect to that of the rat enzyme, and that loss of the enzyme function has been directly linked to an apoptotic effect (data presented in Example VI).

### Example XIV

### Modeling Interaction of Compounds with the Active Site of RabGGT

This example demonstrates that compounds with apoptotic activity and RabGGT inhibitory activity have the potential to block the active site of the RabGGT enzyme.

Methods

The program Insight (Accelrys, Inc., San Diego, Calif.) was used to visualize and compare possible binding interactions of compounds with the active site of RabGGT. The putative binding pocket for inhibitors of RabGGT activity can be hypothesized by comparison with farnesyl transferase (FT), a closely related enzyme that has very similar structure and function (Long et al., 2002, Nature 419:645). The structure of FT in complex with known inhibitory compounds has been determined (for example Long et a.,2001, Proc. Natl. Acad. Sci. USA, 98:12948; Bell et al., 2002, J. Med. Chem. 45:2388).

Results and Conclusions

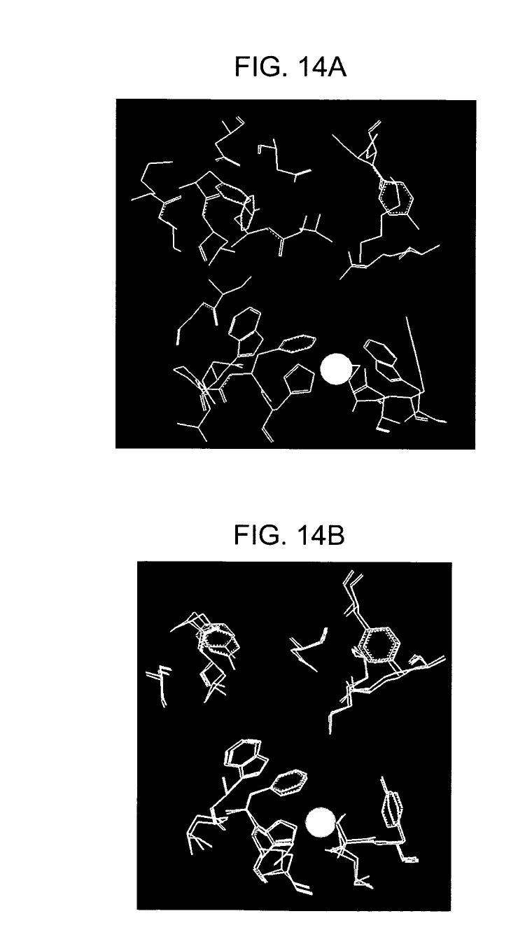

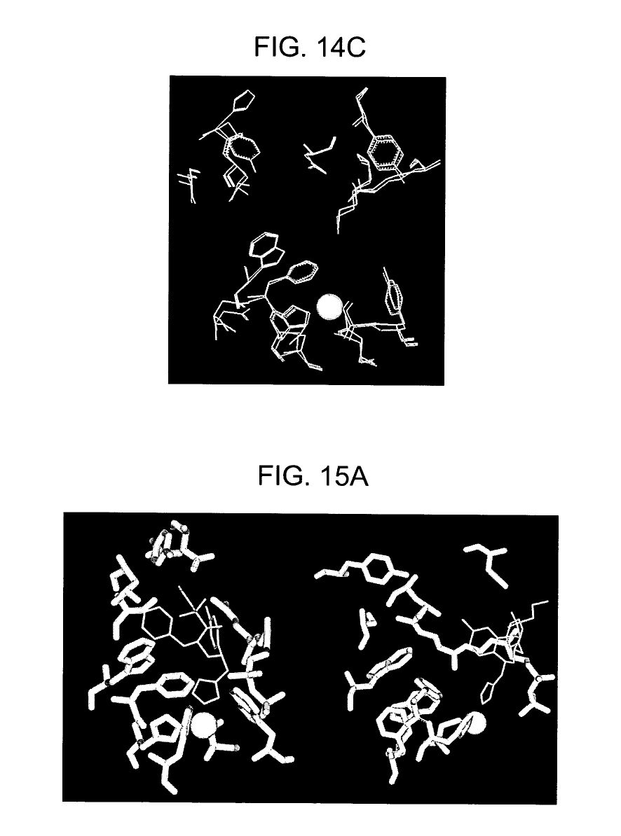

The active site of RabGGT contains binding sites for a prenyl moiety and the peptide substrate of the enzyme. The crystal structure of the RabGGT complex from R. norvegicus is available in the Protein Data Bank as 1DCE (Zhang et al., 2000, Structure 8:241). In the enzyme from R. norvegicus, the active site is composed of residues His B290, Cys B240, Asp B238, Tyr B241, Trp B244, Phe B289, Trp B52, Ser B48, Leu B45, Tyr B44, Asp A61, Arg B144, and Lys A105, where A refers to the alpha subunit and B to the beta subunit (FIG. 14a). The derivation of the 3-dimensional model of the human enzyme from the rat enzyme crystal structure resulted in no significant change to the pocket. The pockets are constitutively identical: the only changes seen were those expected from use of different optimization procedures, which is known to result in slight shifts in amino acid side chain positions (FIG. 14b).

The binding pocket of the predicted human RabGGT enzyme is large and substantially open to solvent on one side (the left side in FIGS. 14a-c). It contains a bound atom of zinc, coordinated by histidine B290, cysteine B240, and aspartic acid B238, identical to the motif found in the rat protein. The floor of the pocket (at the base in FIGS. 14a-c) is composed of phenylalanine B289 and tryptophan B52, and the back of the pocket (to the rear in FIGS. 14a-c) of leucine B45, serine B48, and tyrosine B44. In the crystal structure, the top of the pocket (at the top in FIGS. 14a-c) contains a substantial quantity of bound water molecules in addition to aspartic acid A6 1; the homology model maintains this empty pocket that is occupied by the water molecules in the crystal structure. RabGGT contains substantial functional, sequence, and structural similarities to farnesyl transferase (FT). In FT, the side of the pocket opposite to that exposed to bulk solvent is known to be a binding site for a prenyl group. The geranyl-geranyl prenyl group that is bound and transferred by RabGGT should occupy the analogous location (to the right in FIGS. 14a-c) (Zhang et al., 2000, Structure 8:241).

There is good indication that compounds 7A through 7T would bind in this pocket. FT and RabGGT are similar in the structure of their active sites and in their mechanism of substrate modification (Long et al., 2002, Nature 419:645). Compounds 7A through 7T show the ability to inhibit FT with high potency (Table 8), indicating that they bind to the enzyme. Crystal structures of FT in complex with compounds structurally similar to 7A through 7H have been reported (Bell et al., 2002, J. Med. Chem. 45:2388). Like 7A through 7H, these compounds contain an imidazole ring, a cyanobenzene, and an aromatic moiety, and they have been found to occlude the peptide-substrate binding site of the FT enzyme. The imidazole ring functions in its well-known role as a ligand for zinc, while the cyanobenzene moiety was found to form hydrophobic contacts with the prenyl group. As noted, the RabGGT pocket also contains a zinc ion at the analogous position, and a similar prenyl group is expected to bind to the pocket in the analogous location. The imidazole and cyanobenzene moieties of 7A through 7H are predicted to orient the compounds in an analogous manner within the RabGGT pocket, occluding the peptide-binding site of the enzyme. All the compounds have additional aromatic moieties that may form significant interactions with the enzymes. However, the substrate binding sites of FT and RabGGT have some differences that are expected to have a substantial influence on the type of molecules that can function as effective and specific inhibitors. The binding site of FT is more hydrophobic and, in particular, is more aromatic. It has been determined that the aromatic “back” region of the FT pocket is constrained and places strict orientation demands on ligands of high affinity (Bell et al., 2002, J. Med. Chem 45:2388). The differences between the pockets of FT and RabGGT in this region, in particular the substitution of tryptophan B602 by leucine B54, would be expected to alter the binding specificity by making fewer requirements on orientation and aromaticity. Consequently, compounds of high-affinity for FT might not bind as tightly, if at all, to RabGGT and conversely, specific inhibitors of RabGGT can be designed.

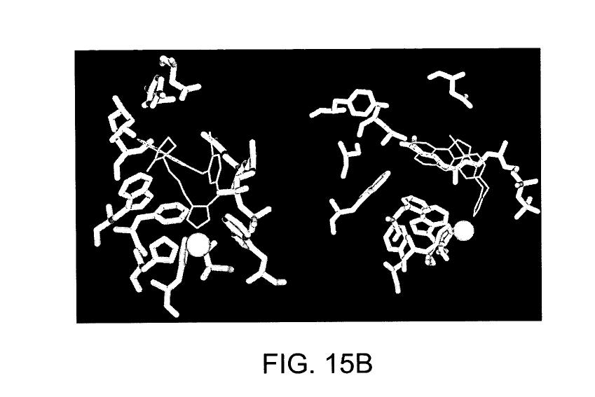

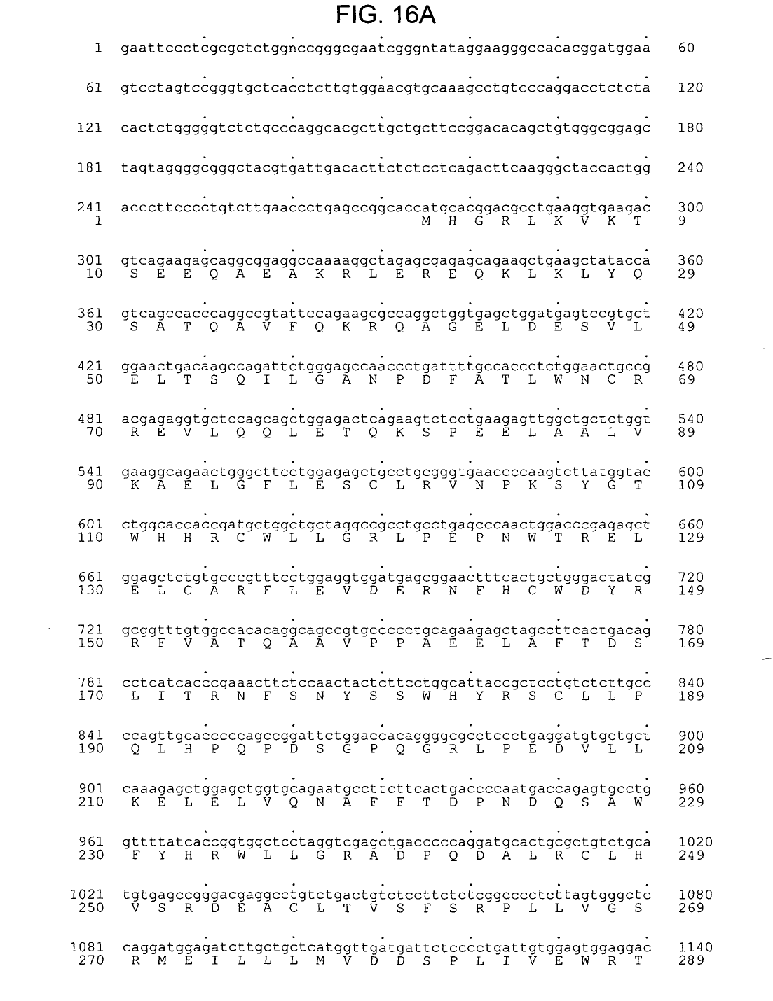

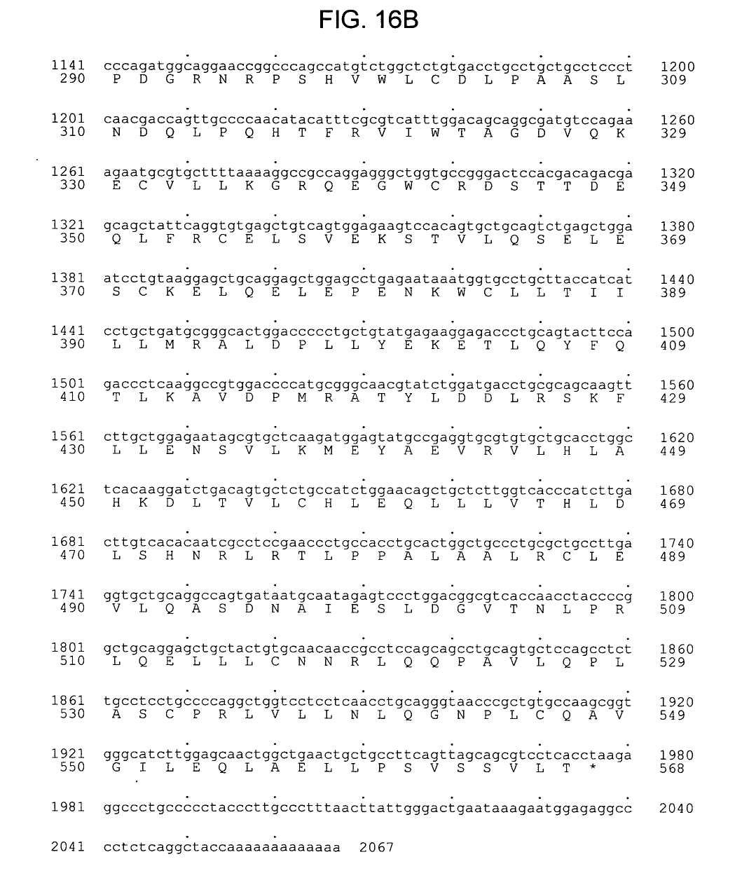

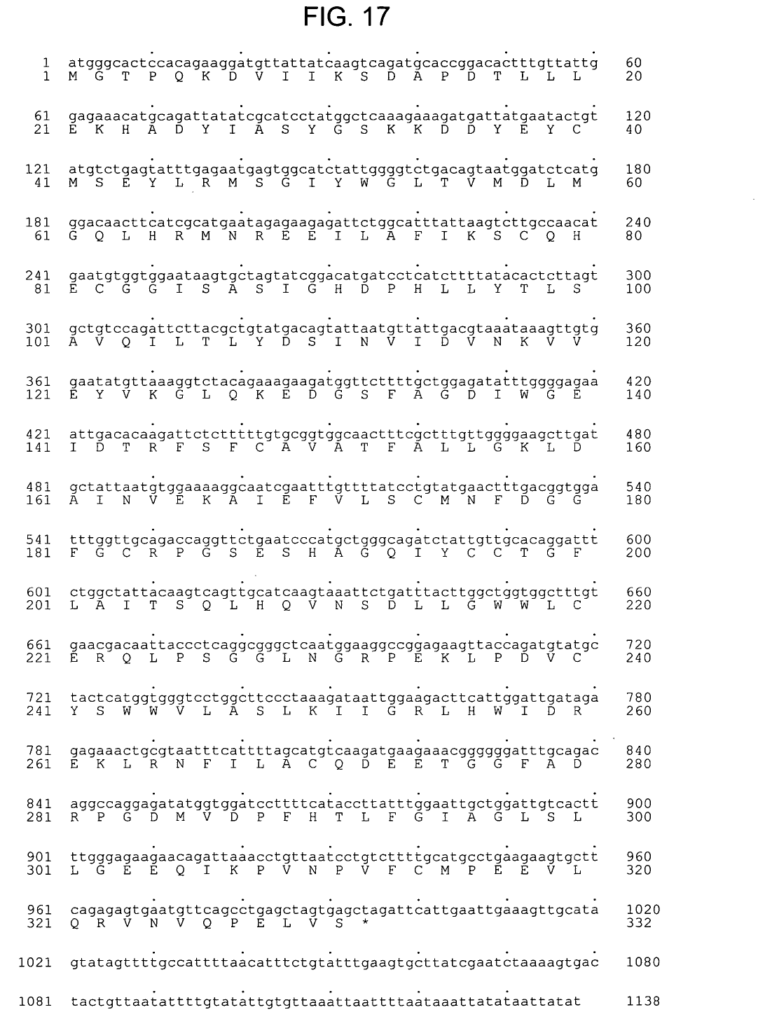

FIG. 15A depicts two views of compound 7H docked into the putative binding site of RABGGT. The left view is facing directly into the cavity opening viewed from outside of the protein, the right is viewed from a 90 degree rotation. The protein residues are heavy sticks.

The ligand is represented by thin sticks. The putative bound atom of zinc is represented as a sphere.

FIG. 15B depicts analogous views of the binding site of the crystal structure of the complex between farnesyl transferase (FT) and the FT inhibitor U66 (PDB 1LD7; Bell et al. (2002) J. Med. Chem. 45:2388). The views show similar binding patterns between the putative Rab ligand and the Rab binding site and that of the FT ligand and the FT binding site. Both show a liganding of an imidazole group to an atom of zinc, a close packing of a cyanophenyl group with a bound prenyl group (shown at the right hand side of the left images and in the middle of the right images) and additional hydrophobic functionality, a phenyl group in the putative Rab ligand and a napthyl group in the FT ligand.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

The entire disclosure of each document cited (including patents, patent applications, journal articles, abstracts, laboratory manuals, books, Genbank Accession Numbers, SWISS-PROT Accession Numbers, or other disclosures) in the Background of the Invention, Detailed Description, and Examples is hereby incorporated herein by reference. Further, the hard copy of the sequence listing submitted herewith and the corresponding computer readable form are both incorporated herein by reference in their entireties.

### Tables 9a and 9b

Alignment of the indicated polypeptides chains. (a) RatA: R. norvegicus RabGGT alpha chain (SEQ ID NO:19), with HsA: H. sapiens RabGGT alpha chain (SEQ ID NO:16). (b) RatB: R. norvegicus RabGGT beta chain (SEQ ID NO:20), with HsB: H. sapiens RabGGT beta chain (SEQ ID NO:18). “\hat{ }” indicates residues within 5 Angstrom of the binding site. “*” indicates identity. “:” indicates conserved properties.

TABLE 9a

| | |
|---|---|
| RatA | ---HGRLKVKTSEEQAEAKRLEREQKLKLYQSATQAVFQKRQAGELDESVLELTSQILGA |
| HsA | M--HGRLKVKTSEEQAEAKRLEREQKLKLYQSATQAVFQKRQAGELDESVLELTSQILGA |
| | ********************************************************* |
| RatA | NPDFATLWNCRREVLQHLETEKSPEESAALVKAELGFLESCLRVNPKSYGTWHHRCWLLS |
| HsA | NPDFATLWNCRREVLQQLETQKSPEELAALVKAELGFLESCLRVNPKSYGTWHHRCWLLG |
| | ****************:***:***** ********************************. |
| | \hat{ } \hat{ } \hat{ } |
| RatA | RLPEPNWARELELCARFLEADERNFHCWDYRRFVAAQAAVAPAEELAFTDSLITRNFSNY |
| HsA | RLPEPNWTRELELCARFLEVDERNFHCWDYRRFVATQAAVPPAEELAFTDSLITRNFSNY |
| | *******:***********.***************:****.******************* |
| RatA | SSWHYRSCLLPQLHPQPDSGPQGRLPENVLLKELELVQNAFFTDPNDQSAWFYHRWLLGR |
| HsA | SSWHYRSCLLPQLHPQPDSGPQGRLPEDVLLKELELVQNAFFTDPNDQSAWFYHRWLLGR |
| | ***************************:******************************** |
| RatA | AEPHDVLCCVHVSREEACLSVCFSRPLTVGSRMGTLLLMVDEAPLSVEWRTPDGRNRPSH |
| HsA | ADPQDALRCLHVSRDEACLTVSFSRPLLVGSRMEILLLMVDDSPLIVEWRTPDGRNRPSH |
| | *:*:*.* *:****:****:*.***** ***** ******::** ************** |
| RatA | VWLCDLPAASLNDQLPQNTFRVIWTGSDSQKECVLLKDRPECWCRDSATDEQLFRCELSV |
| HsA | VWLCDLPAASLNDQLPQHTFRVTWTAGDVQKECVLLKGRQEGWCRDSTTDEQLFRCELSV |
| | *************************..* ********.* * *****:************ |
| RatA | EKSTVLQSELESCKELQELEPENKWCLLTIILLMRALDPLLYEKETLQYFSTLKAVDPMR |
| HsA | EKSTVLQSELESCKELQELEPENKWCLLTIILLMRALDPLLYEKETLQYFQTLKAVDPMR |
| | **************************************************.********* |
| RatA | AAYLDDLRSKFLLENSVLKMEYADVRVLHLAHKDLTVLCHLEQLLLVTHLDLSHNRLRAL |
| HsA | ATYLDDLRSKFLLENSVLKMEYAEVRVLHLAHKDLTVLCHLEQLLLVTHLDLSHNRLRTL |
| | *:******************:*************************************:* |
| RatA | PPALAALRCLEVLQASDNALENVDGVANLPRLQELLLCNNRLQQSAAIQPLVSCPRLVLL |
| HsA | PPALAALRCLEVLQASDNAIESLDGVTNLPRLQELLLCNNRLQQPAVLQPLASCPRLVLL |
| | *******************:*.:***.*****************.*.:***.******** |
| RatA | NLQGNSLCQEEGTQERLAEMLPSVSSILT------------------------------- |
| HsA | NLQGNPLCQAVGTLEQLAELLPSVSSVLT------------------------------- |
| | *****.*** ** *:***:******:** |

TABLE 9b

| | |
|---|---|
| RatB | -------------------------------TQQKDVTTKSDAPDTLLLEKHADYIAS |
| HsB | -----------------------------MGTPQKDVIIKSDAPDTLLLEKHADYIAS |
| | * **** ******************** |
| RatB | YGSKKDDYEYCMSEYLRMSGVYWGLTVMDLMGQLHRMNKEEILVFIKSCQHECGGVSASI |
| HsB | YGSKKDDYEYCMSEYLRMSGIYWGLTVMDLMGQLHRMNREETLAFIKSCQHECGGISASI |
| | ********************:*****************:****.***********:**** |
| | \hat{ } \hat{ }\hat{ } \hat{ } |
| RatB | GHDPHLLYTLSAVQILTLYDSIHVINVDKVVAYVQSLQKEDGSFAGDIWGEIDTRFSFCA |
| HsB | GHDPHLLYTLSAVQILTLYDSINVIDVNKVVEYVKGLQKEDGSFAGDIWGEIDTRFSFCA |
| | **********************:**:*:*** **:.************************ |
| | \hat{ } |
| RatB | VATLALLGKLDAINVEKATEFVLSCMNFDGGFGCRPGSESHAGQIYCCTGFLAITSQLHQ |
| HsB | VATLALLGKLDAINVEKAIEFVLSCMNFDGGFGCRPGSESHAGQIYCCTGFLAITSQLHQ |
| | ************************************************************ |
| RatB | VNSDLLGWWLCERQLPSGGLNGRPEKLPDVCYSWWVLASLKIIGRLHWIDREKLRSFILA |
| HsB | VNSDLLGWWLCERQLPSGGLNGRPEKLPDVCYSWWVLASLKIIGRLHWIDREKLRNFILA |
| | *******************************************************.**** |
| | \hat{ } \hat{ }\hat{ } |
| RatB | CQDEETGGFADRPGDMVDPFHTLFGIAGLSLLGEEQIKPVSPVFCMPEEVLQRVNVQPEL |
| HsB | CQDEETGGFADRPGDMVDPFHTLFGIAGLSLLGEEQIKPVNPVFCMPEEVLQRVNVQPEL |
| | ****************************************.******************* |
| | \hat{ } \hat{ } \hat{ }\hat{ } |
| RatB | VS- |
| HsB | VS- |
| | ** |

### Tables 10a and 10b

Alignment of the polypeptides indicated. (a) RatA: R. norvegicus RabGGT alpha chain (SEQ ID NO:19), with CeA: C. elegans RabGGT alpha chain (SEQ ID NO:2 1). (b) RatB: R. norvegicus RabGGT beta chain (SEQ ID NO:20), with CeB: C. elegans RabGGT beta chain (SEQ ID NO:22). “\hat{ }” indicates residues within 5 Angstrom of the binding site. “*” indicates identity. “:” indicates conserved properties.

TABLE 10a (i)

| | |
|---|---|
| RatA | -HGRLKVKTSEEQAEAKRLEREQKLKLYQSATQAVFQKRQAGELDESVLELTSQILGANP |
| CeA | MHFVKKVPTTEEEKAAKQKEHTKRSQQFLHVRDKIVAKREKGEYDDEILSLTQAILEKNA |
| | * ** *:**: **: *: :: : : . : :. **: ** *:.:*.**. ** *. |
| RatA | DFATLWNCRREVLQ-HLET---------------EKSPEESAALVKAELGFLE-SCLRVN |
| CeA | DIYTFWNIRRTTIELRMEANEKVQQSADAEEEEKTKSSQKIENLLAGEL-FLSYECIKSN |
| | *: *:** ** .:: ::*: **.:: *: .** **. .*:: * |
| | \hat{ } |
| RatA | PKSYGTWHHRCWLLSRLPEPNWARELELCARFLEADERNFHCWDYRRFVAAQAAVAPAEE |
| CeA | PKSYSAWYQRAWALQRQSAPDFKKELALCEKALQLDCRNFHCWDHRRIVARMAKRSEAEE |
| | ****.:*::*.* *.* . *:: :** ** : *: * *******:**:** * : *** |
| | \hat{ } \hat{ } |
| RatA | LAFTDSLITRNFSNYSSWHYRSCLLPQLHPQPDSGPQGRLPENVLLKELELVQNAFFTDP |
| CeA | LEFSNKLINDNFSNYSAWHYRSIALKNIHRDEKTGAP-KIDDELIASELQKVKNAFFMDA |
| | * *::.**. ******:***** * ::* : .:*. :: :::: .**: *:**** *. |
| RatA | NDQSAWFYHRWLLGPAEPRDVLCC-VHVSREEACLSVCFSRPLTVGSRNGTL--LLMVDE |
| CeA | EDQSAWTYTRWLLEVGSGKEFLRPESHTPIELISASFRGNNTTLVFSRAVTIQFLLTFVD |
| | :***** * **** .. ::.* *.. * . *. ... * ** *: ** . : |
| RatA | APLSVEWRTPDGRNRPSHVWLCDLPAASLNDQLPQHTFRVIWTGSDSQKECVLLKDRPEC |
| CeA | TENTTGWRAFSSTS-PNPT------SSRVWQYLSDTPLRVV-TSNPTDLENISWTELNEQ |
| | : :. **: .. . *. . :: : : *.: .:**: *.. :: * : .: * |

TABLE 10a (ii)

| | |
|---|---|
| RatA | WCRDSATDEQLFRCELSVEKSTVLQSELESCKELQELEPENKWCLLTIILLMRALDPLLY |
| CeA | PYVNLDRLKTIYDV-VEVPQPAYIGELLEDCKQLIELEPKNKWPLYMRTLVLLEYQPIKS |
| | : : :: :.* :.: : . **.**:* ****:*** * *:: :*: |
| RatA | EKETLQYFSTLKA-VDPMRAAYLDDLRSK----FLLENSVLKMEYADVRVLHLAHKDLTV |
| CeA | YEEIIKNLENLSENLDPKRSELYKSLISRQNLNFSIREQFERILGPDTDWLTCRYSKLTS |
| | :* :: :..*. ** *: ..* *: * :.:.. :: .*. * :..** |
| RatA | LCHLEQLL-LVTHLDLSHNRLRALPPALAALRCLEVLQASDNALENVDGVANLPRLQELL |
| CeA | LEGVEYLAGFVGSADFSGNRLKEIQR--IVLPNLKSLTINENPIESLPPSPCLSHLTFFS |
| | * :* * :* *:* ***: : .* *: * .:*.:*.: . *.:* : |
| RatA | LCNNRLQQSAAIQPLV-SCPRLVLLNLQGNSLCQE-EGIQERLAEMLPSVSSILT----- |
| CeA | IAGTQIASVSAVMPFFQTIPSLDRLVFCETPLVEKTEELRAQLPGVRLIPHWL------- |
| | :...:: . :*: *:. : * * * : ..* :: * :: :*. : : |

TABLE 10b

| | |
|---|---|
| 1DCE | ------------------------------------------------------------ |
| Ceb | -------------------------------------------------------MSFAG |
| 1DCE | ---TQQKDVTIKSDAPDTLLLEKHADYIASYGSKKDDYEYCMSEYLRMSGVYWGLTVMDL |
| Ceb | LLDFARKDVDLPQNSPNELLKDLHANFINQYEKNKNSYHYIMAEHLRVSGIYWCVNAMDL |
| | :*** : .::*: ** : **::* .* .:*:.*.* *:*:**:**:** :..*** |
| | \hat{ } \hat{ }\hat{ } \hat{ } |
| 1DCE | MGQLHRMNKEEILVFIKSCQHECGGVSASIGHDPHLLYTLSAVQILTLYDSIHVINVDKV |
| Ceb | SKQLERMSTEEIVNYVLGCRNTDGGYGPAPGHDSHLLHTLCAVQTLIIFNSIEKADADTI |
| | **.**..***: :: .*:: ** ..: ***.***:**.*** * :::**. :.*.: |
| 1DCE | VAYVQSLQKEDGSFAGDIWGEIDTRFSFCAVATLALLGKLDAINVEKAIEFVLSCMNFDG |
| Ceb | SEYVKGLQQEDGSFCGDLSGEVDTRFTLCSLATCHLLGRLSTLNIDSAVRFLMRCYNTDG |
| | **:.**:*****.**: **:****::*::** ***:*.::*::.*:.*:: * * ** |
| | \hat{ } |
| 1DCE | GFGCRPGSESHAGQIYCCTGFLAITSQLHQVNSDLLGWWLCERQLPSGGLNGRPEKLPDV |
| Ceb | GFGTRPGSESHSGQIYCCVGALAIAGRLDEIDRDRTAEWLAFRQCDSGGLNGRPEKLPDV |
| | *** *******:******.* ***:.:*.::: * . **. ** ************** |
| | \hat{ } |
| 1DCE | CYSWWVLASLKIIGRLHWIDREKLRSFILACQDEETGGFADRPGDMVDPFHTLFGIAGLS |
| Ceb | CYSWWVLASLAILGRLNFIDSDAMKKFIYACQDDETGGFADRPGDCADPFHTVFGIAALS |
| | ********** *:***::** : ::.** ****:*********** .*****:****.** |
| | \hat{ }\hat{ } \hat{ } \hat{ } \hat{ }\hat{ } |
| 1DCE | LLGEEQIKPVSPVFCMPEEVLQRVNVQPELVS |
| Ceb | LFGDDTLESVDPIFCMTKRCLGDKQVEMYY-- |
| | *:*:: ::.*.*:***.:. * :*: |

TABLE 11

| | Residue/Residue | | | | |
|---|---|---|---|---|---|
| Atom No. | Position | Atom Type | X Coord. | Y Coord. | Z Coord. |
| 1 | MET1 | N | 40.653 | 31.02 | 43.155 |
| 2 | MET1 | CA | 41.733 | 30.626 | 42.225 |
| 3 | MET1 | CB | 42.562 | 29.486 | 42.796 |
| 4 | MET1 | CG | 43.356 | 29.876 | 44.046 |
| 5 | MET1 | SD | 44.746 | 31.016 | 43.814 |
| 6 | MET1 | CE | 43.928 | 32.613 | 44.03 |
| 7 | MET1 | C | 41.152 | 30.205 | 40.88 |
| 8 | MET1 | O | 39.987 | 30.488 | 40.569 |
| 9 | HIS2 | N | 41.95 | 29.458 | 40.134 |
| 10 | HIS2 | CA | 41.596 | 29.033 | 38.771 |
| 11 | HIS2 | CB | 42.849 | 28.472 | 38.107 |
| 12 | HIS2 | CG | 44.026 | 29.429 | 38.102 |
| 13 | HIS2 | ND1 | 45.264 | 29.172 | 38.567 |
| 14 | HIS2 | CE1 | 46.039 | 30.263 | 38.397 |
| 15 | HIS2 | NE2 | 45.28 | 31.216 | 37.81 |
| 16 | HIS2 | CD2 | 44.038 | 30.716 | 37.619 |
| 17 | HIS2 | C | 40.506 | 27.962 | 38.757 |
| 18 | HIS2 | O | 40.782 | 26.764 | 38.881 |
| 19 | GLY3 | N | 39.271 | 28.422 | 38.637 |
| 20 | GLY3 | CA | 38.109 | 27.533 | 38.582 |
| 21 | GLY3 | C | 37.613 | 27.167 | 39.979 |
| 22 | GLY3 | O | 36.847 | 26.208 | 40.142 |
| 23 | ARG4 | N | 38.005 | 27.948 | 40.972 |
| 24 | ARG4 | CA | 37.645 | 27.604 | 42.351 |
| 25 | ARG4 | CB | 38.847 | 27.832 | 43.257 |
| 26 | ARG4 | CG | 39.963 | 26.85 | 42.922 |
| 27 | ARG4 | CD | 39.495 | 25.415 | 43.127 |
| 28 | ARG4 | NE | 40.539 | 24.455 | 42.74 |
| 29 | ARG4 | CZ | 40.293 | 23.154 | 42.577 |
| 30 | ARG4 | NH1 | 39.058 | 22.681 | 42.765 |
| 31 | ARG4 | NH2 | 41.279 | 22.326 | 42.226 |
| 32 | ARG4 | C | 36.45 | 28.404 | 42.847 |
| 33 | ARG4 | O | 36.592 | 29.5 | 43.402 |
| 34 | LEU5 | N | 35.275 | 27.83 | 42.652 |
| 35 | LEU5 | CA | 34.042 | 28.459 | 43.133 |
| 36 | LEU5 | CB | 32.87 | 27.909 | 42.325 |
| 37 | LEU5 | CG | 31.585 | 28.69 | 42.577 |
| 38 | LEU5 | CD1 | 31.774 | 30.171 | 42.266 |
| 39 | LEU5 | CD2 | 30.432 | 28.116 | 41.762 |
| 40 | LEU5 | C | 33.859 | 28.174 | 44.625 |
| 41 | LEU5 | O | 33.747 | 27.017 | 45.052 |
| 42 | LYS6 | N | 33.824 | 29.245 | 45.399 |
| 43 | LYS6 | CA | 33.719 | 29.156 | 46.862 |
| 44 | LYS6 | CB | 34.246 | 30.49 | 47.403 |
| 45 | LYS6 | OG | 34.657 | 30.483 | 48.878 |
| 46 | LYS6 | CD | 33.484 | 30.587 | 49.849 |
| 47 | LYS6 | CE | 33.971 | 30.644 | 51.29 |
| 48 | LYS6 | NZ | 34.837 | 31.811 | 51.512 |
| 49 | LYS6 | C | 32.27 | 28.908 | 47.299 |
| 50 | LYS6 | O | 31.495 | 29.848 | 47.504 |
| 51 | VAL7 | N | 31.904 | 27.64 | 47.395 |
| 52 | VAL7 | CA | 30.565 | 27.283 | 47.882 |
| 53 | VAL7 | CB | 29.863 | 26.409 | 46.842 |
| 54 | VAL7 | CG1 | 28.404 | 26.162 | 47.222 |
| 55 | VAL7 | CG2 | 29.927 | 27.039 | 45.457 |
| 56 | VAL7 | C | 30.666 | 26.525 | 49.203 |
| 57 | VAL7 | O | 30.582 | 27.136 | 50.279 |
| 58 | LYS8 | N | 31.179 | 25.307 | 49.097 |
| 59 | LYS8 | CA | 31.24 | 24.358 | 50.223 |
| 60 | LYS8 | CB | 31.282 | 22.949 | 49.649 |
| 61 | LYS8 | CG | 30.039 | 22.674 | 48.813 |
| 62 | LYS8 | CD | 30.044 | 21.261 | 48.242 |
| 63 | LYS8 | CE | 28.78 | 20.993 | 47.431 |
| 64 | LYS8 | NZ | 28.78 | 19.623 | 46.893 |
| 65 | LYS8 | C | 32.426 | 24.565 | 51.165 |
| 66 | LYS8 | O | 32.687 | 23.736 | 52.04 |
| 67 | THR9 | N | 33.147 | 25.655 | 50.966 |
| 68 | THR9 | CA | 34.276 | 25.989 | 51.832 |
| 69 | THR9 | CB | 35.443 | 26.463 | 50.975 |
| 70 | THR9 | OG1 | 35.045 | 27.648 | 50.305 |
| 71 | THR9 | CG2 | 35.826 | 25.426 | 49.923 |
| 72 | THR9 | C | 33.877 | 27.077 | 52.829 |
| 73 | THR9 | O | 34.734 | 27.613 | 53.54 |
| 74 | SER10 | N | 32.62 | 27.49 | 52.776 |
| 75 | SER10 | CA | 32.126 | 28.488 | 53.727 |
| 76 | SER10 | CB | 31.028 | 29.322 | 53.074 |
| 77 | SER10 | OG | 29.901 | 28.485 | 52.855 |
| 78 | SER10 | C | 31.569 | 27.824 | 54.98 |
| 79 | SER10 | O | 30.988 | 26.734 | 54.922 |
| 80 | GLU11 | N | 31.487 | 28.619 | 56.037 |
| 81 | GLU11 | CA | 30.953 | 28.127 | 57.32 |
| 82 | GLU11 | CB | 31.451 | 29.033 | 58.442 |
| 83 | GLU11 | CG | 32.976 | 29.108 | 58.496 |
| 84 | GLU11 | CD | 33.598 | 27.741 | 58.789 |
| 85 | GLU11 | OE1 | 33.833 | 27.465 | 59.957 |
| 86 | GLU11 | OE2 | 33.935 | 27.06 | 57.831 |
| 87 | GLU11 | C | 29.422 | 28.105 | 57.312 |
| 88 | GLU11 | O | 28.797 | 27.338 | 58.054 |
| 89 | GLU12 | N | 28.873 | 28.7 | 56.264 |
| 90 | GLU12 | CA | 27.431 | 28.778 | 56.014 |
| 91 | GLU12 | CB | 27.107 | 30.028 | 55.189 |
| 92 | GLU12 | CG | 27.208 | 31.353 | 55.958 |
| 93 | GLU12 | CD | 28.646 | 31.859 | 56.096 |
| 94 | GLU12 | OE1 | 29.481 | 31.411 | 55.317 |
| 95 | GLU12 | OE2 | 28.924 | 32.504 | 57.096 |
| 96 | GLU12 | C | 26.907 | 27.542 | 55.276 |
| 97 | GLU12 | O | 25.853 | 27.612 | 54.635 |
| 98 | GLN13 | N | 27.726 | 26.505 | 55.185 |
| 99 | GLN13 | CA | 27.257 | 25.216 | 54.675 |
| 100 | GLN13 | CB | 28.354 | 24.607 | 53.805 |
| 101 | GLN13 | CG | 28.79 | 25.554 | 52.684 |
| 102 | GLN13 | CD | 27.804 | 25.627 | 51.511 |
| 103 | GLN13 | OE1 | 28.034 | 24.995 | 50.472 |
| 104 | GLN13 | NE2 | 26.775 | 26.45 | 51.643 |
| 105 | GLN13 | C | 26.891 | 24.283 | 55.83 |
| 106 | GLN13 | O | 26.528 | 23.124 | 55.596 |
| 107 | ALA14 | N | 27.051 | 24.783 | 57.05 |
| 108 | ALA14 | CA | 26.655 | 24.074 | 58.276 |
| 109 | ALA14 | CB | 25.136 | 23.938 | 58.312 |
| 110 | ALA14 | C | 27.309 | 22.706 | 58.395 |
| 111 | ALA14 | O | 26.639 | 21.669 | 58.356 |
| 112 | GLU15 | N | 28.629 | 22.71 | 58.441 |
| 113 | GLU15 | CA | 29.374 | 21.458 | 58.596 |
| 114 | GLU15 | CB | 29.979 | 21.029 | 57.258 |
| 115 | GLU15 | CG | 28.925 | 20.696 | 56.197 |
| 116 | GLU15 | CD | 28.065 | 19.498 | 56.609 |
| 117 | GLU15 | OE1 | 27.15 | 19.183 | 55.861 |
| 118 | GLU15 | OE2 | 28.516 | 18.771 | 57.485 |
| 119 | GLU15 | C | 30.468 | 21.636 | 59.641 |
| 120 | GLU15 | O | 31.247 | 22.596 | 59.59 |
| 121 | ALA16 | N | 30.475 | 20.747 | 60.618 |
| 122 | ALA16 | CA | 31.461 | 20.839 | 61.701 |
| 123 | ALA16 | CB | 30.865 | 20.228 | 62.964 |
| 124 | ALA16 | C | 32.744 | 20.112 | 61.327 |
| 125 | ALA16 | O | 32.85 | 18.902 | 61.557 |
| 126 | LYS17 | N | 33.757 | 20.898 | 60.992 |
| 127 | LYS17 | CA | 35.038 | 20.384 | 60.473 |
| 128 | LYS17 | CB | 35.821 | 19.703 | 61.593 |
| 129 | LYS17 | CG | 36.221 | 20.685 | 62.685 |
| 130 | LYS17 | CD | 37.179 | 21.744 | 62.154 |
| 131 | LYS17 | CE | 37.533 | 22.751 | 63.239 |
| 132 | LYS17 | NZ | 36.321 | 23.416 | 63.742 |
| 133 | LYS17 | C | 34.835 | 19.393 | 59.33 |
| 134 | LYS17 | O | 34.484 | 19.784 | 58.21 |
| 135 | ARG18 | N | 35.076 | 18.126 | 59.639 |
| 136 | ARG18 | CA | 34.983 | 17.02 | 58.672 |
| 137 | ARG18 | CB | 33.555 | 16.922 | 58.139 |
| 138 | ARG18 | OG | 32.539 | 16.738 | 59.259 |
| 139 | ARG18 | CD | 31.115 | 16.866 | 58.736 |
| 140 | ARG18 | NE | 30.145 | 16.788 | 59.839 |
| 141 | ARG18 | CZ | 29.063 | 16.006 | 59.802 |
| 142 | ARG18 | NH1 | 28.228 | 15.974 | 60.843 |
| 143 | ARG18 | NH2 | 28.821 | 15.251 | 58.727 |
| 144 | ARG18 | C | 35.941 | 17.232 | 57.508 |
| 145 | ARG18 | O | 35.532 | 17.176 | 56.341 |
| 146 | LEU19 | N | 37.217 | 17.383 | 57.821 |
| 147 | LEU19 | CA | 38.216 | 17.626 | 56.776 |
| 148 | LEU19 | CB | 39.294 | 18.555 | 57.322 |
| 149 | LEU19 | CG | 40.188 | 19.086 | 56.206 |
| 150 | LEU19 | CD1 | 39.359 | 19.788 | 55.134 |
| 151 | LEU19 | CD2 | 41.256 | 20.022 | 56.758 |
| 152 | LEU19 | C | 38.82 | 16.302 | 56.311 |
| 153 | LEU19 | O | 39.966 | 15.956 | 56.621 |
| 154 | GLU20 | N | 38.012 | 15.553 | 55.586 |
| 155 | GLU20 | CA | 38.441 | 14.242 | 55.117 |
| 156 | GLU20 | CB | 37.259 | 13.285 | 55.047 |
| 157 | GLU20 | CG | 36.922 | 12.721 | 56.43 |
| 158 | GLU20 | CD | 37.967 | 11.695 | 56.89 |
| 159 | GLU20 | OE1 | 37.553 | 10.572 | 57.15 |
| 160 | GLU20 | OE2 | 39.15 | 11.962 | 56.735 |
| 161 | GLU20 | C | 39.191 | 14.32 | 53.804 |
| 162 | GLU20 | O | 39.491 | 15.417 | 53.319 |
| 163 | ARG21 | N | 39.718 | 13.156 | 53.438 |
| 164 | ARG21 | CA | 40.594 | 12.947 | 52.271 |
| 165 | ARG21 | CB | 40.106 | 13.73 | 51.054 |
| 166 | ARG21 | CG | 38.694 | 13.277 | 50.69 |
| 167 | ARG21 | CD | 37.921 | 14.351 | 49.933 |
| 168 | ARG21 | NE | 36.489 | 14.008 | 49.895 |
| 169 | ARG21 | CZ | 35.601 | 14.459 | 50.788 |
| 170 | ARG21 | NH1 | 35.978 | 15.32 | 51.738 |
| 171 | ARG21 | NH2 | 34.322 | 14.086 | 50.7 |
| 172 | ARG21 | C | 42.011 | 13.319 | 52.69 |
| 173 | ARG21 | O | 42.95 | 13.337 | 51.885 |
| 174 | GLU22 | N | 42.179 | 13.227 | 54 |
| 175 | GLU22 | CA | 43.451 | 13.502 | 54.655 |
| 176 | GLU22 | CB | 43.173 | 14.109 | 56.032 |
| 177 | GLU22 | CG | 42.12 | 13.321 | 56.807 |
| 178 | GLU22 | CD | 41.759 | 14.027 | 58.115 |
| 179 | GLU22 | OE1 | 40.721 | 13.683 | 58.669 |
| 180 | GLU22 | OE2 | 42.607 | 14.746 | 58.625 |
| 181 | GLU22 | C | 44.252 | 12.211 | 54.738 |
| 182 | GLU22 | O | 45.486 | 12.239 | 54.779 |
| 183 | GLN23 | N | 43.565 | 11.123 | 54.43 |
| 184 | GLN23 | CA | 44.193 | 9.812 | 54.312 |
| 185 | GLN23 | CB | 43.112 | 8.742 | 54.446 |
| 186 | GLN23 | OG | 42.268 | 8.926 | 55.706 |
| 187 | GLN23 | CD | 40.867 | 9.443 | 55.366 |
| 188 | GLN23 | OE1 | 40.706 | 10.528 | 54.78 |
| 189 | GLN23 | NE2 | 39.881 | 8.634 | 55.708 |
| 190 | GLN23 | C | 44.858 | 9.694 | 52.946 |
| 191 | GLN23 | O | 45.968 | 9.158 | 52.843 |
| 192 | LYS24 | N | 44.33 | 10.45 | 51.994 |
| 193 | LYS24 | CA | 44.931 | 10.514 | 50.664 |
| 194 | LYS24 | CB | 43.893 | 11.031 | 49.677 |
| 195 | LYS24 | CG | 44.535 | 11.295 | 48.322 |
| 196 | LYS24 | CD | 43.591 | 12.014 | 47.368 |
| 197 | LYS24 | CE | 44.325 | 12.404 | 46.09 |
| 198 | LYS24 | NZ | 45.481 | 13.265 | 46.402 |
| 199 | LYS24 | C | 46.113 | 11.47 | 50.685 |
| 200 | LYS24 | O | 47.16 | 11.167 | 50.1 |
| 201 | LEU25 | N | 46.041 | 12.449 | 51.573 |
| 202 | LEU25 | CA | 47.154 | 13.382 | 51.743 |
| 203 | LEU25 | CB | 46.684 | 14.573 | 52.567 |
| 204 | LEU25 | CG | 45.593 | 15.352 | 51.844 |
| 205 | LEU25 | CD1 | 45.027 | 16.453 | 52.731 |
| 206 | LEU25 | CD2 | 46.11 | 15.926 | 50.529 |
| 207 | LEU25 | C | 48.328 | 12.704 | 52.437 |
| 208 | LEU25 | O | 49.436 | 12.76 | 51.894 |
| 209 | LYS26 | N | 48.044 | 11.819 | 53.38 |
| 210 | LYS26 | CA | 49.12 | 11.068 | 54.039 |
| 211 | LYS26 | CB | 48.577 | 10.457 | 55.322 |
| 212 | LYS26 | CG | 48.181 | 11.536 | 56.323 |
| 213 | LYS26 | CD | 47.574 | 10.921 | 57.579 |
| 214 | LYS26 | CE | 46.356 | 10.073 | 57.234 |
| 215 | LYS26 | NZ | 45.742 | 9.501 | 58.439 |
| 216 | LYS26 | C | 49.698 | 9.967 | 53.153 |
| 217 | LYS26 | O | 50.908 | 9.723 | 53.218 |
| 218 | LEU27 | N | 48.923 | 9.49 | 52.192 |
| 219 | LEU27 | CA | 49.45 | 8.521 | 51 .225 |
| 220 | LEU27 | CB | 48.272 | 7.84 | 50.536 |
| 221 | LEU27 | CG | 48.735 | 6.807 | 49.513 |
| 222 | LEU27 | CD1 | 49.589 | 5.727 | 50.169 |
| 223 | LEU27 | CD2 | 47.543 | 6.184 | 48.795 |
| 224 | LEU27 | C | 50.323 | 9.218 | 50.184 |
| 225 | LEU27 | O | 51.427 | 8.739 | 49.894 |
| 226 | TYR28 | N | 49.963 | 10.449 | 49.865 |
| 227 | TYR28 | CA | 50.736 | 11.291 | 48.949 |
| 228 | TYR28 | CB | 49.875 | 12.534 | 48.717 |
| 229 | TYR28 | CG | 50.383 | 13.618 | 47.77 |
| 230 | TYR28 | CO1 | 49.901 | 13.677 | 46.468 |
| 231 | TYR28 | CE1 | 50.336 | 14.681 | 45.611 |
| 232 | TYR28 | CZ | 51.246 | 15.628 | 46.064 |
| 233 | TYR28 | OH | 51.649 | 16.648 | 45.23 |
| 234 | TYR28 | CE2 | 51.722 | 15.578 | 47.367 |
| 235 | TYR28 | CD2 | 51.283 | 14.576 | 48.223 |
| 236 | TYR28 | C | 52.071 | 11.668 | 49.588 |
| 237 | TYR28 | O | 53.133 | 11.412 | 49.002 |
| 238 | GLN29 | N | 52.012 | 11.973 | 50.875 |
| 239 | GLN29 | CA | 53.208 | 12.313 | 51.649 |
| 240 | GLN29 | CB | 52.768 | 12.743 | 53.04 |
| 241 | GLN29 | CG | 51.923 | 14.008 | 53.01 |
| 242 | GLN29 | CD | 51.212 | 14.145 | 54.351 |
| 243 | GLN29 | OE1 | 50.063 | 14.599 | 54.429 |
| 244 | GLN29 | NE2 | 51.865 | 13.631 | 55.378 |
| 245 | GLN29 | C | 54.145 | 11.124 | 51.799 |
| 246 | GLN29 | O | 55.306 | 11.232 | 51.39 |
| 247 | SER30 | 53.59 | 49.958 | 52.097 | |
| 248 | SER30 | CA | 54.429 | 8.777 | 52.335 |
| 249 | SER30 | CB | 53.602 | 7.745 | 53.087 |
| 250 | SER30 | OG | 53.224 | 8.332 | 54.326 |
| 251 | SER30 | C | 54.976 | 8.167 | 51.051 |
| 252 | SER30 | O | 56.117 | 7.686 | 51.052 |
| 253 | ALA31 | N | 54.311 | 8.413 | 49.935 |
| 254 | ALA31 | CA | 54.847 | 7.961 | 48.653 |
| 255 | ALA31 | CB | 53.723 | 7.938 | 47.622 |
| 256 | ALA31 | C | 55.966 | 8.886 | 48.187 |
| 257 | ALA31 | O | 57 | 8.388 | 47.727 |
| 258 | THR32 | N | 55.899 | 10.143 | 48.595 |
| 259 | THR32 | CA | 56.954 | 11.105 | 48.259 |
| 260 | THR32 | CB | 56.387 | 12.513 | 48.416 |
| 261 | THR32 | OG1 | 55.249 | 12.637 | 47.575 |
| 262 | THR32 | CG2 | 57.389 | 13.582 | 48.003 |
| 263 | THR32 | C | 58.164 | 10.934 | 49.176 |
| 264 | THR32 | O | 59.308 | 10.998 | 48.705 |
| 265 | GLN33 | N | 57.913 | 10.463 | 50.387 |
| 266 | GLN33 | CA | 58.996 | 10.184 | 51.33 |
| 267 | GLN33 | CB | 58.392 | 10.07 | 52.725 |
| 268 | GLN33 | CG | 57.783 | 11.402 | 53.151 |
| 269 | GLN33 | CD | 56.975 | 11.254 | 54.437 |
| 270 | GLN33 | OE1 | 56.121 | 10.367 | 54.565 |
| 271 | GLN33 | NE2 | 57.181 | 12.2 | 55.336 |
| 272 | GLN33 | C | 59.718 | 8.894 | 50.962 |
| 273 | GLN33 | O | 60.957 | 8.892 | 50.913 |
| 274 | ALA34 | N | 58.971 | 7.95 | 50.409 |
| 275 | ALA34 | CA | 59.568 | 6.707 | 49.922 |
| 276 | ALA34 | CB | 58.464 | 5.684 | 49.69 |
| 277 | ALA34 | G | 60.351 | 6.933 | 48.634 |
| 278 | ALA34 | O | 61.491 | 6.462 | 48.535 |
| 279 | VAL35 | N | 59.891 | 7.865 | 47.814 |
| 280 | VAL35 | CA | 60.644 | 8.228 | 46.612 |
| 281 | VAL35 | CB | 59.814 | 9.173 | 45.752 |
| 282 | VAL35 | CG1 | 60.666 | 9.824 | 44.671 |
| 283 | VAL35 | CG2 | 58.628 | 8.458 | 45.129 |
| 284 | VAL35 | C | 61.954 | 8.92 | 46.961 |
| 285 | VAL35 | O | 63.002 | 8.48 | 46.473 |
| 286 | PHE36 | N | 61.943 | 9.761 | 47.984 |
| 287 | PHE36 | CA | 63.167 | 10.481 | 48.344 |
| 288 | PHE36 | CB | 62.82 | 11.684 | 49.212 |
| 289 | PHE36 | CG | 62.135 | 12.83 | 48.472 |
| 290 | PHE36 | CD1 | 61.298 | 13.696 | 49.163 |
| 291 | PHE36 | OE1 | 60.678 | 14.743 | 48.495 |
| 292 | PHE36 | CZ | 60.896 | 14.927 | 47.136 |
| 293 | PHE36 | CE2 | 61.739 | 14.066 | 46.446 |
| 294 | PHE36 | CD2 | 62.362 | 13.021 | 47.115 |
| 295 | PHE36 | C | 64.174 | 9.605 | 49.079 |
| 296 | PHE36 | O | 65.381 | 9.784 | 48.87 |
| 297 | GLN37 | N | 63.717 | 8.563 | 49.754 |
| 298 | GLN37 | CA | 64.677 | 7.682 | 50.42 |
| 299 | GLN37 | CB | 64.069 | 7.128 | 51.704 |
| 300 | GLN37 | CG | 62.783 | 6.351 | 51.47 |
| 301 | GLN37 | CD | 62.066 | 6.161 | 52.799 |
| 302 | GLN37 | OE1 | 60.833 | 6.065 | 52.855 |
| 303 | GLN37 | NE2 | 62.85 | 6.168 | 53.863 |
| 304 | GLN37 | C | 65.194 | 6.582 | 49.492 |
| 305 | GLN37 | O | 66.371 | 6.218 | 49.604 |
| 306 | LYS38 | N | 64.466 | 6.29 | 148.427 |
| 307 | LYS38 | CA | 65 | 5.377 | 47.418 |
| 308 | LYS38 | CB | 63.852 | 4.812 | 46.597 |
| 309 | LYS38 | CG | 62.916 | 3.961 | 47.443 |
| 310 | LYS38 | CD | 61.707 | 3.513 | 46.634 |
| 311 | LYS38 | CE | 60.754 | 2.682 | 47.484 |
| 312 | LYS38 | NZ | 61.43 | 1.484 | 48.004 |
| 313 | LYS38 | C | 65.956 | 6.128 | 46.504 |
| 314 | LYS38 | O | 67.062 | 5.638 | 46.237 |
| 315 | ARG39 | N | 65.674 | 7.407 | 46.327 |
| 316 | ARG39 | CA | 66.528 | 8.285 | 45.528 |
| 317 | ARG39 | CB | 65.786 | 9.608 | 45.381 |
| 318 | ARG39 | CG | 66.475 | 10.59 | 44.442 |
| 319 | ARG39 | CD | 65.692 | 11.898 | 44.407 |
| 320 | ARG39 | NE | 66.223 | 12.832 | 43.402 |
| 321 | ARG39 | CZ | 65.737 | 14.064 | 43.238 |
| 322 | ARG39 | NH1 | 64.791 | 14.519 | 44.063 |
| 323 | ARG39 | NH2 | 66.234 | 14.861 | 42.29 |
| 324 | ARG39 | C | 67.874 | 8.524 | 46.208 |
| 325 | ARG39 | O | 68.909 | 8.289 | 45.571 |
| 326 | GLN40 | N | 67.863 | 8.662 | 47.528 |
| 327 | GLN40 | CA | 69.117 | 8.884 | 48.266 |
| 328 | GLN40 | CB | 68.815 | 9.633 | 49.564 |
| 329 | GLN40 | CG | 68.052 | 8.783 | 50.574 |
| 330 | GLN40 | CD | 67.561 | 9.644 | 51.734 |
| 331 | GLN40 | OE1 | 67.735 | 9.301 | 52.909 |
| 332 | GLN40 | NE2 | 66.843 | 10.695 | 51.381 |
| 333 | GLN40 | C | 69.871 | 7.582 | 48.561 |
| 334 | GLN40 | O | 71.033 | 7.629 | 48.981 |
| 335 | ALA41 | N | 69.251 | 6.445 | 48.28 |
| 336 | ALA41 | CA | 69.937 | 5.157 | 48.382 |
| 337 | ALA41 | CB | 68.955 | 4.121 | 48.916 |
| 338 | ALA41 | C | 70.486 | 4.698 | 47.029 |
| 339 | ALA41 | O | 71.154 | 3.66 | 46.947 |
| 340 | GLY42 | N | 70.172 | 5.441 | 45.977 |
| 341 | GLY42 | CA | 70.682 | 5.123 | 44.638 |
| 342 | GLY42 | C | 69.757 | 4.168 | 43.888 |
| 343 | GLY42 | O | 70.156 | 3.534 | 42.903 |
| 344 | GLU43 | N | 68.509 | 4.113 | 44.319 |
| 345 | GLU43 | CA | 67.538 | 3.194 | 43.721 |
| 346 | GLU43 | CB | 66.577 | 2.715 | 44.801 |
| 347 | GLU43 | CG | 67.297 | 2.019 | 45.947 |
| 348 | GLU43 | CD | 66.284 | 1.643 | 47.023 |
| 349 | GLU43 | OE1 | 65.116 | 1.52 | 46.683 |
| 350 | GLU43 | OE2 | 66.672 | 1.603 | 48.182 |
| 351 | GLU43 | C | 66.732 | 3.886 | 42.633 |
| 352 | GLU43 | O | 65.535 | 4.142 | 42.808 |
| 353 | LEU44 | N | 67.353 | 4.083 | 41.483 |
| 354 | LEU44 | CA | 66.677 | 4.749 | 40.359 |
| 355 | LEU44 | CB | 67.705 | 5.54 | 39.562 |
| 356 | LEU44 | CG | 68.365 | 6.614 | 40.419 |
| 357 | LEU44 | CO1 | 69.482 | 7.309 | 39.651 |
| 358 | LEU44 | CD2 | 67.34 | 7.626 | 40.925 |
| 359 | LEU44 | C | 65.976 | 3.74 | 39.451 |
| 360 | LEU44 | O | 66.282 | 3.62 | 38.261 |
| 361 | ASP45 | N | 65.002 | 3.051 | 40.021 |
| 362 | ASP45 | CA | 64.279 | 2.002 | 39.299 |
| 363 | ASP45 | CB | 64.678 | 0.645 | 39.878 |
| 364 | ASP45 | CG | 64.491 | 0.607 | 41.394 |
| 365 | ASP45 | OD1 | 65.474 | 0.774 | 42.102 |
| 366 | ASP45 | OD2 | 63.357 | 0.407 | 41.809 |
| 367 | ASP45 | C | 62.766 | 2.216 | 39.355 |
| 368 | ASP45 | O | 62.282 | 3.253 | 39.831 |
| 369 | GLU46 | N | 62.03 | 1.164 | 39.029 |
| 370 | GLU46 | CA | 60.569 | 1.259 | 38.905 |
| 371 | GLU46 | CB | 59.99 | 0.088 | 38.099 |
| 372 | GLU46 | CG | 59.955 | −1.256 | 38.835 |
| 373 | GLU46 | CD | 61.224 | −2.072 | 38.594 |
| 374 | GLU46 | OE1 | 61.214 | −2.877 | 37.677 |
| 375 | GLU46 | OE2 | 62.233 | −1.729 | 39.201 |
| 376 | GLU46 | C | 59.822 | 1.364 | 40.239 |
| 377 | GLU46 | O | 58.672 | 1.808 | 40.215 |
| 378 | SER47 | N | 60.487 | 1.206 | 41.376 |
| 379 | SER47 | CA | 59.798 | 1.442 | 42.651 |
| 380 | SER47 | CB | 60.593 | 0.822 | 43.798 |
| 381 | SER47 | OG | 61.847 | 1.486 | 43.909 |
| 382 | SER47 | C | 59.604 | 2.941 | 42.889 |
| 383 | SER47 | O | 58.501 | 3.348 | 43.267 |
| 384 | VAL48 | N | 60.503 | 3.743 | 42.337 |
| 385 | VAL48 | CA | 60.365 | 5.194 | 42.441 |
| 386 | VAL48 | CB | 61.735 | 5.823 | 42.227 |
| 387 | VAL48 | CG1 | 61.654 | 7.343 | 42.186 |
| 388 | VAL48 | CG2 | 62.713 | 5.367 | 43.297 |
| 389 | VAL48 | C | 59.408 | 5.694 | 41.371 |
| 390 | VAL48 | O | 58.499 | 6.475 | 41.681 |
| 391 | LEU49 | N | 59.39 | 4.974 | 40.262 |
| 392 | LEU49 | CA | 58.535 | 5.333 | 39.133 |
| 393 | LEU49 | CB | 58.97 | 4.47 | 37.957 |
| 394 | LEU49 | CG | 58.603 | 5.097 | 36.621 |
| 395 | LEU49 | OD1 | 59.419 | 6.366 | 36.413 |
| 396 | LEU49 | CD2 | 58.864 | 4.12 | 35.48 |
| 397 | LEU49 | C | 57.06 | 5.061 | 39.44 |
| 398 | LEU49 | O | 56.222 | 5.948 | 39.242 |
| 399 | GLU50 | N | 56.797 | 3.989 | 40.17 |
| 400 | GLU50 | CA | 55.415 | 3.643 | 40.52 |
| 401 | GLU50 | CB | 55.322 | 2.133 | 40.728 |
| 402 | GLU50 | CG | 56.119 | 1.664 | 41.939 |
| 403 | GLU50 | CD | 56.406 | 0.168 | 41.847 |
| 404 | GLU50 | OE1 | 56.595 | −0.306 | 40.735 |
| 405 | GLU50 | OE2 | 56.612 | −0.432 | 42.893 |
| 406 | GLU50 | C | 54.902 | 4.393 | 41.753 |
| 407 | GLU50 | O | 53.693 | 4.368 | 42.015 |
| 408 | LEU51 | N | 55.766 | 5.115 | 42.449 |
| 409 | LEU51 | CA | 55.286 | 5.967 | 43.535 |
| 410 | LEU51 | CB | 56.301 | 5.97 | 44.668 |
| 411 | LEU51 | CG | 56.423 | 4.605 | 45.329 |
| 412 | LEU51 | OD1 | 57.6 | 4.577 | 46.295 |
| 413 | LEU51 | CD2 | 55.129 | 4.217 | 46.036 |
| 414 | LEU51 | C | 55.078 | 7.381 | 43.014 |
| 415 | LEU51 | O | 53.993 | 7.949 | 43.208 |
| 416 | THR52 | N | 55.95 | 7.783 | 42.1 |
| 417 | THR52 | CA | 55.831 | 9.107 | 41.473 |
| 418 | THR52 | CB | 57.125 | 9.492 | 40.758 |
| 419 | THR52 | OG1 | 57.453 | 8.479 | 39.818 |
| 420 | THR52 | CG2 | 58.296 | 9.648 | 41.714 |
| 421 | THR52 | C | 54.69 | 9.156 | 40.467 |
| 422 | THR52 | O | 54.066 | 10.211 | 40.337 |
| 423 | SER53 | N | 54.244 | 8.003 | 39.996 |
| 424 | SER53 | CA | 53.07 | 7.963 | 39.121 |
| 425 | SER53 | CB | 52.986 | 6.583 | 38.476 |
| 426 | SER53 | OC | 52.87 | 5.613 | 39.509 |
| 427 | SER53 | C | 51.762 | 8.256 | 39.859 |
| 428 | SER53 | O | 50.881 | 8.897 | 39.277 |
| 429 | GLN54 | N | 51.732 | 8.049 | 41.166 |
| 430 | GLN54 | CA | 50.515 | 8.354 | 41.916 |
| 431 | GLN54 | CB | 50.509 | 7.501 | 43.177 |
| 432 | GLN54 | CG | 50.595 | 6.019 | 42.839 |
| 433 | GLN54 | CD | 50.702 | 5.198 | 44.119 |
| 434 | GLN54 | OE1 | 49.888 | 5.335 | 45.039 |
| 435 | GLN54 | NE2 | 51.725 | 4.365 | 44.168 |
| 436 | GLN54 | C | 50.506 | 9.824 | 42.306 |
| 437 | GLN54 | O | 49.529 | 10.54 | 42.039 |
| 438 | ILEA55 | N | 51.695 | 10.312 | 42.617 |
| 439 | ILEA55 | CA | 51.835 | 11.687 | 43.091 |
| 440 | ILEA55 | CB | 53.197 | 11.803 | 43.752 |
| 441 | ILEA55 | CG2 | 53.298 | 13.124 | 44.5 |
| 442 | ILEA55 | OG1 | 53.417 | 10.646 | 44.715 |
| 443 | ILEA55 | CD1 | 54.876 | 10.568 | 45.136 |
| 444 | ILEA55 | C | 51.741 | 12.694 | 41.951 |
| 445 | ILEA55 | O | 51.023 | 13.689 | 42.09 |
| 446 | LEU56 | N | 52.232 | 12.318 | 40.781 |
| 447 | LEU56 | CA | 52.15 | 13.19 | 39.605 |
| 448 | LEU56 | CB | 53.305 | 12.867 | 38.67 |
| 449 | LEU56 | CG | 54.641 | 13.172 | 39.333 |
| 450 | LEU56 | CD1 | 55.801 | 12.611 | 38.527 |
| 451 | LEU56 | CD2 | 54.807 | 14.667 | 39.551 |
| 452 | LEU56 | C | 50.823 | 13.027 | 38.871 |
| 453 | LEU56 | O | 50.382 | 13.961 | 38.19 |
| 454 | GLY57 | N | 50.106 | 11.961 | 39.188 |
| 455 | GLY57 | CA | 48.735 | 11.794 | 38.702 |
| 456 | GLY57 | C | 47.828 | 12.818 | 39.377 |
| 457 | GLY57 | O | 47.03 | 13.488 | 38.711 |
| 458 | ALA58 | N | 48.031 | 13 | 40.674 |
| 459 | ALA58 | CA | 47.297 | 14.026 | 41.428 |
| 460 | ALA58 | CB | 47.194 | 13.566 | 42.879 |
| 461 | ALA58 | C | 47.954 | 15.413 | 41.379 |
| 462 | ALA58 | O | 47.393 | 16.379 | 41.911 |
| 463 | ASN59 | N | 49.113 | 15.505 | 40.747 |
| 464 | ASN59 | CA | 49.849 | 16.769 | 40.637 |
| 465 | ASN59 | CB | 50.54 | 17.031 | 41.973 |
| 466 | ASN59 | OG | 51.275 | 18.373 | 42.02 |
| 467 | ASN59 | OD1 | 51.473 | 19.056 | 41.004 |
| 468 | ASN59 | ND2 | 51.832 | 18.629 | 43.188 |
| 469 | ASN59 | C | 50.893 | 16.689 | 39.525 |
| 470 | ASN59 | O | 52.077 | 16.434 | 39.789 |
| 471 | PRO60 | N | 50.507 | 17.158 | 38.348 |
| 472 | PRO60 | CA | 51.395 | 17.139 | 37.175 |
| 473 | PR060 | CB | 50.48 | 17.388 | 36.018 |
| 474 | PRO60 | CG | 49.117 | 17.82 | 36.534 |
| 475 | PRO60 | CD | 49.189 | 17.722 | 38.046 |
| 476 | PR060 | C | 52.504 | 18.204 | 37.192 |
| 477 | PRO60 | O | 53.34 | 18.238 | 36.283 |
| 478 | ASP61 | N | 52.531 | 19.057 | 38.201 |
| 479 | ASP61 | CA | 53.538 | 20.114 | 38.267 |
| 480 | ASP61 | CB | 52.852 | 21.459 | 38.443 |
| 481 | ASP61 | CG | 52.193 | 21.843 | 37.125 |
| 482 | ASP61 | OD1 | 52.927 | 22.254 | 36.234 |
| 483 | ASP61 | OD2 | 51.025 | 21.515 | 36.953 |
| 484 | ASP61 | C | 54.559 | 19.886 | 39.373 |
| 485 | ASP61 | O | 55.335 | 20.8 | 39.681 |
| 486 | PHE62 | N | 54.549 | 18.711 | 39.984 |
| 487 | PHE62 | CA | 55.586 | 18.388 | 40.973 |
| 488 | PHE62 | CB | 55.057 | 17.277 | 41.876 |
| 489 | PHE62 | CG | 55.701 | 17.16 | 43.259 |
| 490 | PHE62 | CD1 | 54.944 | 16.673 | 44.317 |
| 491 | PHE62 | CE1 | 55.506 | 16.558 | 45.581 |
| 492 | PHE62 | CZ | 56.826 | 16.934 | 45.791 |
| 493 | PHE62 | CE2 | 57.583 | 17.426 | 44.736 |
| 494 | PHE62 | CD2 | 57.02 | 17.541 | 43.471 |
| 495 | PHE62 | C | 56.86 | 17.95 | 40.242 |
| 496 | PHE62 | O | 57.216 | 16.764 | 40.224 |
| 497 | ALA63 | N | 57.653 | 18.947 | 39.876 |
| 498 | ALA63 | CA | 58.828 | 18.75 | 39.018 |
| 499 | ALA63 | CB | 59.249 | 20.105 | 38.46 |
| 500 | ALA63 | C | 60.017 | 18.089 | 39.704 |
| 501 | ALA63 | O | 60.829 | 17.463 | 39.017 |
| 502 | THR64 | N | 59.961 | 17.957 | 41.018 |
| 503 | THR64 | CA | 61.016 | 17.233 | 41.725 |
| 504 | THR64 | CB | 60.927 | 17.575 | 43.206 |
| 505 | THR64 | OG1 | 61.077 | 18.982 | 43.337 |
| 506 | THR64 | CG2 | 62.034 | 16.906 | 44.01 |
| 507 | THR64 | C | 60.855 | 15.728 | 41.518 |
| 508 | THR64 | O | 61.854 | 15.04 | 41 .275 |
| 509 | LEU65 | N | 59.624 | 15.306 | 41 .271 |
| 510 | LEU65 | CA | 59.362 | 13.895 | 41.001 |
| 511 | LEU65 | CB | 57.995 | 13.532 | 41.551 |
| 512 | LEU65 | CG | 57.951 | 13.757 | 43.057 |
| 513 | LEU65 | CD1 | 56.569 | 13.454 | 43.597 |
| 514 | LEU65 | CD2 | 58.991 | 12.912 | 43.783 |
| 515 | LEU65 | C | 59.446 | 13.607 | 39.508 |
| 516 | LEU65 | O | 59.743 | 12.472 | 39.119 |
| 517 | TRP66 | N | 59.445 | 14.663 | 38.711 |
| 518 | TRP66 | CA | 59.762 | 14.518 | 37.29 |
| 519 | TRP66 | CB | 59.236 | 15.716 | 36.509 |
| 520 | TRP66 | CG | 57.732 | 15.771 | 36.339 |
| 521 | TRP66 | CD1 | 56.893 | 16.775 | 36.765 |
| 522 | TRP66 | NE1 | 55.625 | 16.46 | 36.403 |
| 523 | TRP66 | CE2 | 55.582 | 15.281 | 35.758 |
| 524 | TRP66 | CZ2 | 54.544 | 14.556 | 35.195 |
| 525 | TRP66 | CH2 | 54.808 | 13.342 | 34.575 |
| 526 | TRP66 | CZ3 | 56.108 | 12.852 | 34.514 |
| 527 | TRP66 | CE3 | 57.154 | 13.574 | 35.073 |
| 528 | TRP66 | CD2 | 56.896 | 14.787 | 35.693 |
| 529 | TRP66 | C | 61.271 | 14.404 | 37.092 |
| 530 | TRP66 | O | 61.705 | 13.643 | 36.219 |
| 531 | ASN67 | N | 62.04 | 14.936 | 38.033 |
| 532 | ASN67 | CA | 63.489 | 14.714 | 38.034 |
| 533 | ASN67 | CB | 64.164 | 15.667 | 39.012 |
| 534 | ASN67 | GG | 63.947 | 17.128 | 38.648 |
| 535 | ASN67 | OD1 | 63.841 | 17.496 | 37.473 |
| 536 | ASN67 | ND2 | 63.977 | 17.959 | 39.675 |
| 537 | ASN67 | C | 63.804 | 13.297 | 38.492 |
| 538 | ASN67 | O | 64.677 | 12.645 | 37.903 |
| 539 | CYS68 | N | 62.958 | 12.758 | 39.356 |
| 540 | CYS68 | CA | 63.113 | 11.367 | 39.787 |
| 541 | CYS68 | CB | 62.19 | 11.103 | 40.967 |
| 542 | CYS68 | SG | 62.506 | 12.099 | 42.438 |
| 543 | CYS68 | C | 62.777 | 10.399 | 38.659 |
| 544 | CYS68 | O | 63.586 | 9.503 | 38.389 |
| 545 | ARG69 | N | 61.794 | 10.741 | 37.839 |
| 546 | ARG69 | CA | 61.474 | 9.9 | 36.68 |
| 547 | ARG69 | CB | 60.095 | 10.27 | 36.155 |
| 548 | ARG69 | CG | 59.026 | 10.002 | 37.203 |
| 549 | ARG69 | CD | 57.633 | 10.262 | 36.647 |
| 550 | ARG69 | NE | 57.328 | 9.369 | 35.519 |
| 551 | ARG69 | CZ | 56.5 | 8.328 | 35.628 |
| 552 | ARG69 | NH1 | 56.247 | 7.554 | 34.571 |
| 553 | ARG69 | NH2 | 55.919 | 8.062 | 36.797 |
| 554 | ARG69 | C | 62.497 | 10.045 | 35.557 |
| 555 | ARG69 | O | 62.819 | 9.044 | 34.909 |
| 556 | ARG70 | N | 63.174 | 11.18 | 35.497 |
| 557 | ARG70 | CA | 64.273 | 11.339 | 34.543 |
| 558 | ARG70 | CB | 64.652 | 12.813 | 34.459 |
| 559 | ARG70 | CG | 63.817 | 13.518 | 33.403 |
| 560 | ARG70 | CD | 64.152 | 14.998 | 33.28 |
| 561 | ARG70 | NE | 63.384 | 15.803 | 34.238 |
| 562 | ARG70 | CZ | 62.513 | 16.729 | 33.832 |
| 563 | ARG70 | NH1 | 62.35 | 16.958 | 32.527 |
| 564 | ARG70 | NH2 | 61.823 | 17.44 | 34.725 |
| 565 | ARG70 | C | 65.499 | 10.53 | 34.946 |
| 566 | ARG70 | O | 66.071 | 9.84 | 34.094 |
| 567 | GLU71 | N | 65.728 | 10.403 | 36.241 |
| 568 | GLU71 | CA | 66.874 | 9.635 | 36.731 |
| 569 | GLU7I | CB | 67.137 | 10.077 | 38.162 |
| 570 | GLU71 | CG | 67.534 | 11.546 | 38.196 |
| 571 | GLU71 | CD | 67.372 | 12.096 | 39.608 |
| 572 | GLU71 | OE1 | 66.439 | 11.673 | 40.277 |
| 573 | GLU71 | OE2 | 68.106 | 13.013 | 39.949 |
| 574 | GLU71 | C | 66.603 | 8.135 | 36.687 |
| 575 | GLU71 | O | 67.472 | 7.377 | 36.239 |
| 576 | VAL72 | N | 65.347 | 7.763 | 36.875 |
| 577 | VAL72 | CA | 64.952 | 6.359 | 36.753 |
| 578 | VAL72 | CB | 63.543 | 6.191 | 37.316 |
| 579 | VAL72 | CG1 | 62.954 | 4.833 | 36.955 |
| 580 | VAL72 | CG2 | 63.511 | 6.411 | 38.823 |
| 581 | VAL72 | C | 64.963 | 5.915 | 35.297 |
| 582 | VAL72 | O | 65.538 | 4.866 | 34.987 |
| 583 | LEU73 | N | 64.605 | 6.818 | 34.398 |
| 584 | LEU73 | CA | 64.592 | 6.466 | 32.98 |
| 585 | LEU73 | CB | 63.706 | 7.436 | 32.205 |
| 586 | LEU73 | CG | 62.358 | 6.823 | 31.819 |
| 587 | LEU73 | CD1 | 61.513 | 6.447 | 33.033 |
| 588 | LEU73 | CD2 | 61.575 | 7.764 | 30.911 |
| 589 | LEU73 | C | 65.989 | 6.457 | 32.38 |
| 590 | LEU73 | O | 66.269 | 5.559 | 31.582 |
| 591 | GLN74 | N | 66.91 | 7.236 | 32.924 |
| 592 | GLN74 | CA | 68.289 | 7.195 | 32.427 |
| 593 | GLN74 | CB | 68.987 | 8.495 | 32.804 |
| 594 | GLN74 | CG | 68.389 | 9.663 | 32.028 |
| 595 | GLN74 | CD | 68.938 | 10.988 | 32.545 |
| 596 | GLN74 | OE1 | 70.088 | 11.078 | 32.991 |
| 597 | GLN74 | NE2 | 68.087 | 11.998 | 32.522 |
| 598 | GLN74 | C | 69.052 | 5.996 | 32.979 |
| 599 | GLN74 | O | 69.75 | 5.315 | 32.214 |
| 600 | GLN75 | N | 68.668 | 5.562 | 34.169 |
| 601 | GLN75 | CA | 69.263 | 4.356 | 34.74 |
| 602 | GLN75 | CB | 68.913 | 4.305 | 36.223 |
| 603 | GLN75 | CG | 69.492 | 3.08 | 36.926 |
| 604 | GLN75 | CD | 71.018 | 3.121 | 36.954 |
| 605 | GLN75 | OE1 | 71.615 | 3.822 | 37.781 |
| 606 | GLN75 | NE2 | 71.63 | 2.363 | 36.06 |
| 607 | GLN75 | C | 68.732 | 3.111 | 34.034 |
| 608 | GLN75 | O | 69.532 | 2.28 | 33.578 |
| 609 | LEU76 | N | 67.473 | 3.187 | 33.639 |
| 610 | LEU76 | CA | 66.824 | 2.1 | 32.9 |
| 611 | LEU76 | CB | 65.31 | 2.293 | 32.988 |
| 612 | LEU76 | CG | 64.619 | 1.454 | 34.069 |
| 613 | LEU76 | CD1 | 65.251 | 1.564 | 35.455 |
| 614 | LEU76 | CD2 | 63.136 | 1.797 | 34.139 |
| 615 | LEU76 | C | 67.24 | 42.069 | 31.43 |
| 616 | LEU76 | O | 67.28 | 10.983 | 30.843 |
| 617 | GLU77 | N | 67.80 | 83.16 | 30.935 |
| 618 | GLU77 | CA | 68.31 | 33.201 | 29.558 |
| 619 | GLU77 | CB | 68.34 | 34.649 | 29.082 |
| 620 | GLU77 | CG | 66.93 | 75.128 | 28.743 |
| 621 | GLU77 | CD | 66.88 | 96.644 | 28.596 |
| 622 | GLU77 | OE1 | 67.54 | 27.316 | 29.383 |
| 623 | GLU77 | OE2 | 66.07 | 87.107 | 27.806 |
| 624 | GLU77 | C | 69.69 | 92.58 | 29.432 |
| 625 | GLU77 | O | 70.15 | 22.304 | 28.316 |
| 626 | THR78 | N | 70.33 | 62.311 | 30.559 |
| 627 | THR78 | CA | 71.58 | 11.545 | 30.543 |
| 628 | THR78 | CB | 72.6 | 2.207 | 31.464 |
| 629 | THR78 | OG1 | 72.20 | 41.988 | 32.81 |
| 630 | THR78 | CG2 | 72.70 | 93.707 | 31.218 |
| 631 | THR78 | C | 71.3 | 50.107 | 31.011 |
| 632 | THR78 | O | 72.324 | −0.631 | 31.201 |
| 633 | GLN79 | N | 70.106 | −0.263 | 31.283 |
| 634 | GLN79 | CA | 69.84 | −1.599 | 31.833 |
| 635 | GLN79 | CB | 69.275 | −1.43 | 33.237 |
| 636 | GLN79 | CG | 70.288 | −0.799 | 34.178 |
| 637 | GLN79 | CD | 69.644 | −0.556 | 35.535 |
| 638 | GLN79 | OE1 | 68.737 | 0.275 | 35.667 |
| 639 | GLN79 | NE2 | 70.167 | −1.233 | 36.541 |
| 640 | GLN79 | C | 68.847 | −2.427 | 31.023 |
| 641 | GLN79 | O | 69.016 | −3.647 | 30.897 |
| 642 | LYS80 | N | 67.798 | −1.789 | 30.536 |
| 643 | LYS80 | CA | 66.708 | −2.52 | 29.879 |
| 644 | LYS80 | CB | 65.439 | −1.675 | 29.918 |
| 645 | LYS80 | CG | 64.964 | −1.421 | 31 .344 |
| 646 | LYS80 | CD | 64.719 | −2.726 | 32.094 |
| 647 | LYS80 | CE | 64.104 | −2.476 | 33.465 |
| 648 | LYS80 | NZ | 62.786 | −1.835 | 33.333 |
| 649 | LYS80 | C | 67.016 | −2.878 | 28.433 |
| 650 | LYS80 | O | 67.642 | −2.111 | 27.693 |
| 651 | SER81 | N | 66.515 | −4.036 | 28.038 |
| 652 | SER81 | CA | 66.603 | −4.479 | 26.642 |
| 653 | SER81 | CB | 66.015 | −5.883 | 26.544 |
| 654 | SER81 | OG | 64.636 | −5.801 | 26.877 |
| 655 | SER81 | C | 65.808 | −3.511 | 25.772 |
| 656 | SER81 | O | 64.814 | −2.948 | 26.245 |
| 657 | PRO82 | N | 66.189 | −3.344 | 24.514 |
| 658 | PRO82 | CA | 65.751 | −2.158 | 23.755 |
| 659 | PRO82 | CB | 66.517 | −2.216 | 22.468 |
| 660 | PRO82 | CG | 67.431 | −3.433 | 22.472 |
| 661 | PRO82 | CD | 67.239 | −4.099 | 23.824 |
| 662 | PRO82 | C | 64.244 | −2.083 | 23.478 |
| 663 | PRO82 | O | 63.663 | −1.003 | 23.629 |
| 664 | GLU83 | N | 63.579 | −3.224 | 23.382 |
| 665 | GLU83 | CA | 62.128 | −3.219 | 23.134 |
| 666 | GLU83 | CB | 61.678 | −4.471 | 22.361 |
| 667 | GLU83 | CG | 61.622 | −5.784 | 23.156 |
| 668 | GLU83 | CD | 62.991 | −6.447 | 23.294 |
| 669 | GLU83 | OE1 | 63.347 | −7.205 | 22.407 |
| 670 | GLU83 | OE2 | 63.738 | −6.003 | 24.159 |
| 671 | GLU83 | C | 61.34 | −3.083 | 24.442 |
| 672 | GLU83 | O | 60.24 | −2.52 | 24.445 |
| 673 | GLU84 | N | 62.014 | −3.332 | 25.553 |
| 674 | GLU84 | CA | 61.405 | −3.181 | 26.871 |
| 675 | GLU84 | CB | 62.162 | −4.11 | 27.807 |
| 676 | GLU84 | CG | 61.732 | −4.009 | 29.262 |
| 677 | GLU84 | CD | 62.705 | −4.849 | 30.079 |
| 678 | GLU84 | OE1 | 63.841 | −4.975 | 29.633 |
| 679 | GLU84 | OE2 | 62.305 | −5.362 | 31.114 |
| 680 | GLU84 | C | 61.571 | −1.739 | 27.325 |
| 681 | GLU84 | O | 60.652 | −1.148 | 27.902 |
| 682 | LEU85 | N | 62.621 | −1.123 | 26.811 |
| 683 | LEU85 | CA | 62.88 | 0.289 | 27.061 |
| 684 | LEU85 | CB | 64.347 | 0.53 | 26.73 |
| 685 | LEU85 | CG | 64.786 | 1.941 | 27.084 |
| 686 | LEU85 | CD1 | 64.585 | 2.206 | 28.573 |
| 687 | LEU85 | CD2 | 66.241 | 2.149 | 26.683 |
| 688 | LEU85 | C | 61.987 | 1.159 | 26.179 |
| 689 | LEU85 | O | 61.461 | 2.17 | 26.656 |
| 690 | ALA86 | N | 61 .603 | 0.627 | 25.028 |
| 691 | ALA86 | CA | 60.646 | 1.324 | 24.164 |
| 692 | ALA86 | CB | 60.728 | 0.728 | 22.763 |
| 693 | ALA86 | C | 59.219 | 1.197 | 24.692 |
| 694 | ALA86 | O | 58.455 | 2.169 | 24.621 |
| 695 | ALA87 | N | 58.955 | 0.134 | 25.435 |
| 696 | ALA87 | CA | 57.655 | −0.005 | 26.095 |
| 697 | ALA87 | CB | 57.457 | −1.463 | 26.492 |
| 698 | ALA87 | C | 57.573 | 0.885 | 27.333 |
| 699 | ALA87 | O | 56.533 | 1.516 | 27.562 |
| 700 | LEU88 | N | 58.721 | 1.151 | 27.938 |
| 701 | LEU88 | CA | 58.786 | 2.087 | 29.068 |
| 702 | LEU88 | CB | 60.133 | 1.931 | 29.775 |
| 703 | LEU88 | CG | 60.042 | 1.16 | 31.092 |
| 704 | LEU88 | CD1 | 59.089 | 1.856 | 32.058 |
| 705 | LEU88 | CD2 | 59.64 | −0.3 | 30.904 |
| 706 | LEU88 | C | 58.638 | 3.531 | 28.595 |
| 707 | LEU88 | O | 57.907 | 4.304 | 29.225 |
| 708 | VAL89 | N | 59.101 | 3.808 | 27.387 |
| 709 | VAL89 | CA | 58.939 | 5.143 | 26.805 |
| 710 | VAL89 | CB | 59.923 | 5.275 | 25.646 |
| 711 | VAL89 | CG1 | 59.604 | 6.475 | 24.762 |
| 712 | VAL89 | CG2 | 61.36 | 5.335 | 26.149 |
| 713 | VAL89 | C | 57.516 | 5.387 | 26.305 |
| 714 | VAL89 | O | 56.978 | 6.481 | 26.521 |
| 715 | LYS90 | N | 56.831 | 4.332 | 25.894 |
| 716 | LYS90 | CA | 55.447 | 4.498 | 25.446 |
| 717 | LYS90 | CB | 55.08 | 3.332 | 24.537 |
| 718 | LYS90 | CG | 53.699 | 3.528 | 23.924 |
| 719 | LYS90 | CD | 53.359 | 2.418 | 22.938 |
| 720 | LYS90 | CE | 51.986 | 2.64 | 22.314 |
| 721 | LYS90 | NZ | 51.679 | 1.594 | 21.326 |
| 722 | LYS90 | C | 54.487 | 4.574 | 26.632 |
| 723 | LYS90 | O | 53.552 | 5.386 | 26.608 |
| 724 | ALA91 | N | 54.874 | 3.965 | 27.743 |
| 725 | ALA91 | CA | 54.092 | 4.096 | 28.977 |
| 726 | ALA91 | CB | 54.473 | 2.963 | 29.923 |
| 727 | ALA91 | C | 54.37 | 5.439 | 29.648 |
| 728 | ALA91 | O | 53.458 | 6.05 | 30.219 |
| 729 | GLU92 | N | 55.535 | 5.992 | 29.353 |
| 730 | GLU92 | CA | 55.875 | 7.336 | 29.807 |
| 731 | GLU92 | CB | 57.365 | 7.557 | 29.57 |
| 732 | GLU92 | CG | 57.826 | 8.924 | 30.061 |
| 733 | GLU92 | CD | 57.723 | 8.995 | 31.578 |
| 734 | GLU92 | OE1 | 58.446 | 8.25 | 32.224 |
| 735 | GLU92 | OE2 | 56.968 | 9.825 | 32.061 |
| 736 | GLU92 | C | 55.078 | 8.38 | 29.036 |
| 737 | GLU92 | O | 54.51 | 9.271 | 29.671 |
| 738 | LEU93 | N | 54.824 | 8.14 | 27.758 |
| 739 | LEU93 | CA | 54.006 | 9.076 | 26.974 |
| 740 | LEU93 | CB | 54.212 | 8.792 | 25.491 |
| 741 | LEU93 | CG | 55.632 | 9.145 | 25.074 |
| 742 | LEU93 | CD1 | 55.89 | 8.78 | 23.619 |
| 743 | LEU93 | CD2 | 55.9 | 10.625 | 25.314 |
| 744 | LEU93 | C | 52.526 | 8.956 | 27.319 |
| 745 | LEU93 | O | 51.839 | 9.981 | 27.423 |
| 746 | GLY94 | N | 52.12 | 7.766 | 27.728 |
| 747 | GLY94 | CA | 50.77 | 7.557 | 28.256 |
| 748 | GLY94 | C | 50.555 | 8.376 | 29.525 |
| 749 | GLY94 | O | 49.645 | 9.215 | 29.576 |
| 750 | PHE95 | N | 51.505 | 8.288 | 30.443 |
| 751 | PHE9S | CA | 51.4 | 9.018 | 31 .709 |
| 752 | PHE95 | CB | 52.444 | 8.461 | 32.667 |
| 753 | PHE95 | CG | 52.37 | 9.072 | 34.059 |
| 754 | PHE95 | CD1 | 51.247 | 8.856 | 34.846 |
| 755 | PHE95 | CE1 | 51.171 | 9.414 | 36.114 |
| 756 | PHE9S | CZ | 52.218 | 10.19 | 36.593 |
| 757 | PHE95 | CE2 | 53.339 | 10.41 | 35.804 |
| 758 | PHE95 | CD2 | 53.414 | 9.854 | 34.535 |
| 759 | PHE95 | C | 51.607 | 10.529 | 31.555 |
| 760 | PHE95 | O | 50.902 | 11.296 | 32.222 |
| 761 | LEU96 | N | 52.356 | 10.949 | 30.548 |
| 762 | LEU96 | CA | 52.511 | 12.383 | 30.278 |
| 763 | LEU96 | CB | 53.657 | 12.582 | 29.292 |
| 764 | LEU96 | CG | 55.01 | 12.297 | 29.932 |
| 765 | LEU96 | CD1 | 56.106 | 12.151 | 28.884 |
| 766 | LEU96 | CD2 | 55.372 | 13.366 | 30.952 |
| 767 | LEU96 | C | 51.232 | 12.977 | 29.699 |
| 768 | LEU96 | O | 50.773 | 14.018 | 30.184 |
| 769 | GLU97 | N | 50.511 | 12.178 | 28.929 |
| 770 | GLU97 | CA | 49.229 | 12.628 | 28.386 |
| 771 | GLU97 | CB | 48.834 | 11.694 | 27.248 |
| 772 | GLU97 | CG | 47.492 | 12.087 | 26.641 |
| 773 | GLU97 | CD | 47.143 | 11.133 | 25.506 |
| 774 | GLU97 | OE1 | 46.517 | 11.58 | 24.555 |
| 775 | GLU97 | OE2 | 47.555 | 9.983 | 25.585 |
| 776 | GLU97 | C | 48.145 | 12.615 | 29.457 |
| 777 | GLU97 | O | 47.351 | 13.559 | 29.519 |
| 778 | SER98 | N | 48.3 | 11.745 | 30.442 |
| 779 | SER98 | CA | 47.346 | 11.687 | 31.551 |
| 780 | SER98 | CB | 47.548 | 10.372 | 32.295 |
| 781 | SER98 | OG | 47.35 | 9.313 | 31.368 |
| 782 | SER98 | C | 47.547 | 12.851 | 32.516 |
| 783 | SER98 | O | 46.56 | 13.471 | 32.932 |
| 784 | CYS99 | N | 48.78 | 13.318 | 32.636 |
| 785 | CYS99 | CA | 49.05 | 14.482 | 33.48 |
| 786 | CYS99 | CB | 50.516 | 14.473 | 33.876 |
| 787 | CYS99 | SG | 51.009 | 13.115 | 34.954 |
| 788 | CYS99 | C | 48.701 | 15.789 | 32.775 |
| 789 | CYS99 | O | 48.227 | 16.717 | 33.439 |
| 790 | LEU100 | N | 48.642 | 15.753 | 31.453 |
| 791 | LEU100 | CA | 48.15 | 16.905 | 30.69 |
| 792 | LEU100 | CB | 48.744 | 16.853 | 29.291 |
| 793 | LEU100 | CG | 50.251 | 17.052 | 29.338 |
| 794 | LEU100 | CD1 | 50.885 | 16.82 | 7.975 |
| 795 | LEU100 | CD2 | 50.598 | 18.437 | 29.871 |
| 796 | LEU100 | C | 46.624 | 16.927 | 30.609 |
| 797 | LEU100 | O | 46.032 | 17.981 | 30.357 |
| 798 | ARG101 | N | 45.996 | 15.819 | 30.965 |
| 799 | ARG101 | CA | 44.541 | 15.79 | 31.121 |
| 800 | ARG101 | CB | 44.048 | 14.377 | 30.842 |
| 801 | ARG101 | CG | 44.279 | 13.988 | 29.388 |
| 802 | ARG101 | CD | 43.923 | 12.526 | 29.153 |
| 803 | ARG101 | NE | 42.535 | 12.26 | 29.558 |
| 804 | ARG101 | CZ | 41.576 | 11.903 | 28.701 |
| 805 | ARG101 | NH1 | 41.86 | 11.758 | 27.405 |
| 806 | ARG101 | NH2 | 40.336 | 11.683 | 29.142 |
| 807 | ARG101 | C | 44.134 | 16.204 | 32.535 |
| 808 | ARG101 | O | 42.97 | 16.548 | 32.772 |
| 809 | VAL102 | N | 45.094 | 16.212 | 33.449 |
| 810 | VAL102 | CA | 44.85 | 16.749 | 34.79 |
| 811 | VAL102 | CB | 45.724 | 15.989 | 35.788 |
| 812 | VAL102 | CG1 | 45.539 | 16.509 | 37.21 |
| 813 | VAL102 | CG2 | 45.437 | 14.493 | 35.74 |
| 814 | VAL102 | C | 45.191 | 18.239 | 34.809 |
| 815 | VAL102 | O | 44.574 | 19.022 | 35.544 |
| 816 | ASN103 | N | 46.141 | 18.618 | 33.97 |
| 817 | ASN103 | CA | 46.472 | 20.03 | 33.767 |
| 818 | ASN103 | CB | 47.376 | 20.502 | 34.904 |
| 819 | ASN103 | CG | 47.604 | 22.007 | 34.801 |
| 820 | ASN103 | OD1 | 46.99 | 22.68 | 33.966 |
| 821 | ASN103 | ND2 | 48.587 | 22.492 | 35.537 |
| 822 | ASN103 | C | 47.172 | 20.235 | 32.422 |
| 823 | ASN103 | O | 48.385 | 20.019 | 32.294 |
| 824 | PRO104 | N | 46.439 | 20.82 | 31.486 |
| 825 | PRO104 | CA | 46.962 | 21.09 | 30.137 |
| 826 | PRO104 | CB | 45.746 | 21.394 | 29.316 |
| 827 | PR0104 | CG | 44.546 | 21.556 | 30.237 |
| 828 | PRO104 | CD | 45.041 | 21.234 | 31.637 |
| 829 | PRO104 | C | 47.961 | 22.254 | 30.047 |
| 830 | PRO104 | O | 48.514 | 22.492 | 28.964 |
| 831 | LYS105 | N | 48.18 | 22.975 | 31.137 |
| 832 | LYS105 | CA | 49.199 | 24.028 | 31.157 |
| 833 | LYS105 | 08 | 48.563 | 25.35 | 31.584 |
| 834 | LYS105 | CG | 48.037 | 25.326 | 33.012 |
| 835 | LYS105 | CD | 47.396 | 26.653 | 33.4 |
| 836 | LYS105 | CE | 46.867 | 26.613 | 34.829 |
| 837 | LYS105 | NZ | 46.241 | 27.892 | 35.198 |
| 838 | LYS105 | C | 50.365 | 23.661 | 32.079 |
| 839 | LYS105 | O | 51.108 | 24.545 | 32.525 |
| 840 | SER106 | N | 50.475 | 22.383 | 32.413 |
| 841 | SER106 | CA | 51.538 | 21.926 | 33.315 |
| 842 | SER106 | CB | 51.307 | 20.462 | 33.666 |
| 843 | SER106 | OG | 52.457 | 20.016 | 34.375 |
| 844 | SER106 | C | 52.926 | 22.04 | 32.712 |
| 845 | SER106 | O | 53.342 | 21.16 | 31.951 |
| 846 | TYR107 | N | 53.722 | 22.912 | 33.309 |
| 847 | TYR107 | CA | 55.115 | 23.087 | 32.885 |
| 848 | TYR107 | CB | 55.696 | 24.335 | 33.544 |
| 849 | TYR107 | CG | 55.112 | 25.667 | 33.082 |
| 850 | TYR107 | CD1 | 54.097 | 26.279 | 33.808 |
| 851 | TYR107 | CE1 | 53.576 | 27.494 | 33.385 |
| 852 | TYR107 | CZ | 54.08 | 28.098 | 32.24 |
| 853 | TYR107 | OH | 53.526 | 29.276 | 31.787 |
| 854 | TYR107 | CE2 | 55.103 | 27.497 | 31.52 |
| 855 | TYR107 | CD2 | 55.621 | 26.28 | 31.943 |
| 856 | TYR107 | C | 55.956 | 21.886 | 33.295 |
| 857 | TYR107 | O | 56.807 | 21.445 | 32.513 |
| 858 | GLY108 | N | 55.548 | 21.231 | 34.371 |
| 859 | GLY108 | CA | 56.198 | 19.995 | 34.807 |
| 860 | GLY108 | C | 56.077 | 18.91 | 33.739 |
| 861 | GLY108 | O | 57.09 | 18.499 | 33.154 |
| 862 | THR109 | N | 54.849 | 18.62 | 33.339 |
| 863 | THR109 | CA | 54.631 | 17.534 | 32.383 |
| 864 | THR109 | CB | 53.15 | 17.191 | 32.404 |
| 865 | THR109 | OG1 | 52.775 | 16.927 | 33.749 |
| 866 | THR109 | CG2 | 52.874 | 15.949 | 31.574 |
| 867 | THR109 | C | 55.049 | 17.897 | 30.956 |
| 868 | THR109 | O | 55.648 | 17.05 | 30.279 |
| 869 | TRP110 | N | 54.989 | 19.174 | 30.607 |
| 870 | TRP110 | CA | 55.441 | 19.594 | 29.277 |
| 871 | TRP110 | CB | 54.961 | 21.015 | 28.985 |
| 872 | TRP110 | CG | 53.507 | 21.137 | 28.567 |
| 873 | TRP110 | CD1 | 52.533 | 21.897 | 29.178 |
| 874 | TRP110 | NE1 | 51.371 | 21.738 | 28.496 |
| 875 | TRP110 | CE2 | 51.532 | 20.912 | 27.446 |
| 876 | TRP110 | CZ2 | 50.662 | 20.457 | 26.468 |
| 877 | TRP110 | CH2 | 51.124 | 19.59 | 25.485 |
| 878 | TRP110 | CZ3 | 52.453 | 19.18 | 25.477 |
| 879 | TRP110 | CE3 | 53.332 | 19.632 | 26.454 |
| 880 | TRP110 | CD2 | 52.875 | 20.495 | 27.438 |
| 881 | TRP110 | C | 56.959 | 19.547 | 29.147 |
| 882 | TRP110 | O | 57.448 | 19.012 | 28.145 |
| 883 | HIS111 | N | 57.675 | 19.821 | 30.225 |
| 884 | HIS111 | CA | 59.136 | 19.773 | 30.163 |
| 885 | HIS111 | CB | 59.705 | 20.527 | 31.36 |
| 886 | HIS111 | CG | 61.221 | 20.554 | 31.45 |
| 887 | HIS111 | ND1 | 62.102 | 20.501 | 30.43 |
| 888 | HIS111 | CE1 | 63.357 | 20.554 | 30.921 |
| 889 | HIS111 | NE2 | 63.266 | 20.638 | 32.268 |
| 890 | HIS111 | CD2 | 61.957 | 20.642 | 32.607 |
| 891 | HIS111 | C | 59.638 | 18.334 | 30.165 |
| 892 | HIS111 | O | 60.534 | 18.019 | 29.371 |
| 893 | HIS112 | N | 58.902 | 17.437 | 30.798 |
| 894 | HIS112 | CA | 59.326 | 16.038 | 30.802 |
| 895 | HIS112 | CB | 58.646 | 15.331 | 31.966 |
| 896 | HIS112 | CG | 59.235 | 13.973 | 32.287 |
| 897 | HIS112 | ND1 | 60.228 | 13.722 | 33.16 |
| 898 | HIS112 | CE1 | 60.478 | 12.398 | 33.182 |
| 899 | HIS112 | NE2 | 59.635 | 11.807 | 32.308 |
| 900 | HIS112 | CD2 | 58.862 | 12.764 | 31.748 |
| 901 | HIS112 | C | 58.985 | 15.35 | 29.479 |
| 902 | HIS112 | O | 59.794 | 14.553 | 28.982 |
| 903 | ARG113 | N | 57.969 | 15.848 | 28.791 |
| 904 | ARG113 | CA | 57.638 | 15.283 | 27.483 |
| 905 | ARG113 | CB | 56.165 | 15.532 | 27.186 |
| 906 | ARG113 | CG | 55.722 | 14.677 | 26.008 |
| 907 | ARG113 | CD | 54.223 | 14.765 | 25.757 |
| 908 | ARG113 | NE | 53.847 | 13.857 | 24.663 |
| 909 | ARG113 | CZ | 52.874 | 12.948 | 24.763 |
| 910 | ARG113 | NH1 | 52.149 | 12.874 | 25.879 |
| 911 | ARG113 | NH2 | 52.593 | 12.149 | 23.731 |
| 912 | ARG113 | C | 58.517 | 15.874 | 26.38 |
| 913 | ARG113 | O | 58.925 | 15.135 | 25.474 |
| 914 | CY5114 | N | 59.017 | 17.083 | 26.593 |
| 915 | CY5114 | CA | 59.991 | 17.661 | 25.659 |
| 916 | CY5114 | CB | 60.117 | 19.162 | 25.902 |
| 917 | CY5114 | SG | 58.678 | 20.174 | 25.491 |
| 918 | CY5114 | C | 61.365 | 17.027 | 25.846 |
| 919 | CY5114 | O | 62.069 | 16.776 | 24.862 |
| 920 | TRP115 | N | 61.634 | 16.577 | 27.06 |
| 921 | TRP115 | CA | 62.873 | 15.857 | 27.349 |
| 922 | TRP115 | CB | 62.951 | 15.67 | 28.862 |
| 923 | TRP115 | CG | 64.03 | 14.716 | 29.333 |
| 924 | TRP115 | CD1 | 65.378 | 14.974 | 29.432 |
| 925 | TRP115 | NE1 | 65.998 | 13.853 | 29.879 |
| 926 | TRP115 | CE2 | 65.115 | 12.858 | 30.088 |
| 927 | TRP115 | CZ2 | 65.256 | 11.546 | 30.517 |
| 928 | TRP115 | CH2 | 64.134 | 10.735 | 30.639 |
| 929 | TRP115 | CZ3 | 62.872 | 11.231 | 30.331 |
| 930 | TRP115 | CE3 | 62.721 | 12.541 | 29.896 |
| 931 | TRP115 | CD2 | 63.839 | 13.353 | 29.769 |
| 932 | TRP115 | C | 62.889 | 14.502 | 26.651 |
| 933 | TRP115 | O | 63.794 | 14.239 | 25.846 |
| 934 | LEU116 | N | 61.768 | 13.801 | 26.724 |
| 935 | LEU116 | CA | 61.703 | 12.465 | 26.134 |
| 936 | LEU116 | CB | 60.459 | 11.764 | 26.663 |
| 937 | LEU116 | CG | 60.431 | 10.303 | 26.232 |
| 938 | LEU116 | CD1 | 61.669 | 9.565 | 26.73 |
| 939 | LEU116 | CD2 | 59.166 | 9.619 | 26.73 |
| 940 | LEU116 | C | 61.662 | 12.517 | 24.61 |
| 941 | LEU116 | O | 62.497 | 11.864 | 23.974 |
| 942 | LEU116 | N | 60.961 | 13.497 | 24.063 |
| 943 | LEU117 | CA | 60.844 | 13.619 | 22.6 |
| 944 | LEU117 | CB | 59.565 | 14.375 | 22.236 |
| 945 | LEU117 | CG | 58.33 | 13.481 | 22.079 |
| 946 | LEU117 | CD1 | 58.584 | 12.359 | 21.084 |
| 947 | LEU117 | CD2 | 57.805 | 12.904 | 23.389 |
| 948 | LEU117 | C | 62.052 | 14.316 | 21.964 |
| 949 | LEU117 | O | 62.186 | 14.342 | 20.734 |
| 950 | GLY118 | N | 62.945 | 14.82 | 22.797 |
| 951 | GLY118 | CA | 64.205 | 15.367 | 22.313 |
| 952 | GLY118 | C | 65.251 | 14.265 | 22.199 |
| 953 | GLY118 | O | 66 | 14.224 | 21.214 |
| 954 | ARG119 | N | 65.264 | 13.362 | 23.168 |
| 955 | ARG119 | CA | 66.284 | 12.304 | 23.193 |
| 956 | ARG119 | CB | 66.677 | 12.04 | 24.643 |
| 957 | ARG119 | CG | 65.511 | 11.518 | 25.473 |
| 958 | ARG119 | CD | 65.918 | 11.317 | 26.926 |
| 959 | ARG119 | NE | 67.026 | 10.356 | 27.04 |
| 960 | ARG119 | CZ | 68.172 | 10.619 | 27.676 |
| 961 | ARG119 | NH1 | 69.145 | 9.706 | 27.703 |
| 962 | ARG119 | NH2 | 68.361 | 11.808 | 28.251 |
| 963 | ARG119 | C | 65.871 | 10.988 | 22.523 |
| 964 | ARG119 | O | 66.705 | 10.077 | 22.438 |
| 965 | LEU120 | N | 64.632 | 10.863 | 22.074 |
| 966 | LEU120 | CA | 64.237 | 9.645 | 21.352 |
| 967 | LEU120 | CB | 62.726 | 9.625 | 21.152 |
| 968 | LEU120 | CG | 61.997 | 9.268 | 22.438 |
| 969 | LEU120 | CD1 | 60.486 | 9.295 | 22.234 |
| 970 | LEU120 | CD2 | 62.449 | 7.905 | 22.951 |
| 971 | LEU120 | C | 64.921 | 9.541 | 19.994 |
| 972 | LEU120 | O | 64.866 | 10.47 | 19.184 |
| 973 | PRO121 | N | 65.485 | 8.371 | 19.729 |
| 974 | PRO12I | CA | 66.201 | 8.125 | 18.467 |
| 975 | PRO121 | CB | 66.947 | 6.846 | 18.698 |
| 976 | PRO121 | CG | 66.498 | 6.229 | 20.015 |
| 977 | PRO121 | CD | 65.525 | 7.218 | 20.634 |
| 978 | PRO121 | C | 65.279 | 7.991 | 17.249 |
| 979 | PRO121 | O | 65.731 | 8.147 | 16.109 |
| 980 | GLU122 | N | 64.007 | 7.712 | 17.485 |
| 981 | GLU122 | CA | 63.011 | 7.743 | 16.406 |
| 982 | GLU122 | CB | 62.948 | 6.356 | 15.764 |
| 983 | GLU122 | CG | 62.595 | 6.386 | 14.274 |
| 984 | GLU122 | CD | 61.173 | 6.881 | 14.012 |
| 985 | GLU122 | OE1 | 61.012 | 8.087 | 13.888 |
| 986 | GLU122 | OE2 | 60.294 | 6.042 | 13.877 |
| 987 | GLU122 | C | 61.648 | 8.124 | 16.991 |
| 988 | GLU122 | O | 60.804 | 7.245 | 17.196 |
| 989 | PRO123 | N | 61.443 | 9.407 | 17.25 |
| 990 | PRO123 | CA | 60.234 | 9.86 | 17.944 |
| 991 | PRO123 | CB | 60.569 | 11.238 | 18.422 |
| 992 | PRO123 | CG | 61.889 | 11.676 | 17.808 |
| 993 | PRO123 | CD | 62.361 | 10.513 | 16.96 |
| 994 | PRO123 | C | 59.012 | 9.875 | 17.027 |
| 995 | PRO123 | O | 59.113 | 10.194 | 15.837 |
| 996 | ASN124 | N | 57.865 | 9.525 | 17.588 |
| 997 | ASN124 | CA | 56.624 | 9.531 | 16.807 |
| 998 | ASN124 | CB | 55.643 | 8.532 | 17.417 |
| 999 | ASN124 | CG | 54.414 | 8.344 | 16.524 |
| 1000 | ASN124 | OD1 | 54.074 | 9.207 | 15.703 |
| 1001 | ASN124 | ND2 | 53.732 | 7.232 | 16.724 |
| 1002 | ASN124 | C | 56.02 | 10.931 | 16.787 |
| 1003 | ASN124 | O | 55.146 | 11.264 | 17.597 |
| 1004 | TRP125 | N | 56.283 | 11.629 | 15.697 |
| 1005 | TRP125 | CA | 55.813 | 13.005 | 15.567 |
| 1006 | TRP125 | CB | 56.693 | 13.727 | 14.556 |
| 1007 | TRP125 | CG | 58.12 | 13.919 | 15.033 |
| 1008 | TRP125 | CD1 | 59.271 | 13.659 | 14.322 |
| 1009 | TRP125 | NE1 | 60.339 | 13.96 | 15.104 |
| 1010 | TRP125 | CE2 | 59.946 | 14.4 | 16.313 |
| 1011 | TRP125 | CZ2 | 60.645 | 14.787 | 17.445 |
| 1012 | TRP125 | CH2 | 59.956 | 15.205 | 18.577 |
| 1013 | TRP125 | CZ3 | 58.567 | 15.227 | 18.583 |
| 1014 | TRP125 | CE3 | 57.859 | 14.824 | 17.459 |
| 1015 | TRP125 | CD2 | 58.541 | 14.406 | 16.327 |
| 1016 | TRP125 | C | 54.343 | 13.124 | 15.179 |
| 1017 | TRP125 | O | 53.71 | 14.098 | 15.606 |
| 1018 | THR126 | N | 53.733 | 12.046 | 14.711 |
| 1019 | THR126 | CA | 52.309 | 12.124 | 14.372 |
| 1020 | THR126 | CB | 51.953 | 11.086 | 13.313 |
| 1021 | THR126 | OG1 | 52.041 | 9.785 | 13.876 |
| 1022 | THR126 | CG2 | 52.89 | 11.163 | 12.113 |
| 1023 | THR126 | C | 51.467 | 11.918 | 15.627 |
| 1024 | THR126 | O | 50.421 | 12.56 | 15.771 |
| 1025 | ARG127 | N | 52.072 | 11.304 | 16.633 |
| 1026 | ARG127 | CA | 51.42 | 11.171 | 17.937 |
| 1027 | ARG127 | CB | 52.129 | 10.063 | 18.712 |
| 1028 | ARG127 | CG | 51.631 | 9.955 | 20.149 |
| 1029 | ARG127 | CD | 52.406 | 8.897 | 20.926 |
| 1030 | ARG127 | NE | 52.217 | 7.562 | 20.335 |
| 1031 | ARG127 | CZ | 53.161 | 6.618 | 20.334 |
| 1032 | ARG127 | NH1 | 52.898 | 5.411 | 19.828 |
| 1033 | ARG127 | NH2 | 54.356 | 6.868 | 20.874 |
| 1034 | ARG127 | C | 51.524 | 12.472 | 18.723 |
| 1035 | ARG127 | O | 50.556 | 12.874 | 19.378 |
| 1036 | GLU128 | N | 52.551 | 13.251 | 18.426 |
| 1037 | GLU128 | CA | 52.748 | 14.508 | 19.151 |
| 1038 | GLU128 | CB | 54.218 | 14.896 | 19.076 |
| 1039 | GLU128 | CG | 55.143 | 13.723 | 19.38 |
| 1040 | GLU128 | CD | 54.91 | 13.144 | 20.77 |
| 1041 | GLU128 | OE1 | 54.929 | 13.924 | 21.708 |
| 1042 | GLU128 | OE2 | 54.929 | 11.925 | 20.878 |
| 1043 | GLU128 | C | 51.899 | 15.613 | 18.53 |
| 1044 | GLU128 | O | 51.288 | 16.408 | 19.257 |
| 1045 | LEU129 | N | 51.662 | 15.497 | 17.233 |
| 1046 | LEU129 | CA | 50.782 | 16.452 | 16.557 |
| 1047 | LEU129 | CB | 51.068 | 16.43 | 15.061 |
| 1048 | LEU129 | CG | 52.483 | 16.907 | 14.756 |
| 1049 | LEU129 | CD1 | 52.797 | 16.775 | 13.27 |
| 1050 | LEU129 | CD2 | 52.695 | 18.341 | 15.227 |
| 1051 | LEU129 | C | 49.319 | 16.108 | 16.803 |
| 1052 | LEU129 | O | 48.504 | 17.019 | 16.987 |
| 1053 | GLU130 | N | 49.045 | 14.842 | 17.073 |
| 1054 | GLU130 | CA | 47.681 | 14.446 | 17.422 |
| 1055 | GLU130 | CB | 47.537 | 12.943 | 17.211 |
| 1056 | GLU130 | CG | 46.086 | 12.494 | 17.341 |
| 1057 | GLU130 | CD | 45.235 | 13.14 | 16.25 |
| 1058 | GLU130 | OE1 | 45.743 | 13.273 | 15.145 |
| 1059 | GLU130 | OE2 | 44.074 | 13.409 | 16.517 |
| 1060 | GLU130 | C | 47.368 | 14.799 | 18.873 |
| 1061 | GLU130 | O | 46.247 | 15.236 | 19.153 |
| 1062 | LEU131 | N | 48.4 | 14.871 | 19.699 |
| 1063 | LEU131 | CA | 48.248 | 15.308 | 21.087 |
| 1064 | LEU131 | CB | 49.599 | 15.155 | 21.775 |
| 1065 | LEU131 | CG | 49.526 | 15.567 | 23.238 |
| 1066 | LEU131 | CD1 | 48.847 | 14.479 | 24.06 |
| 1067 | LEU131 | CD2 | 50.916 | 15.855 | 23.788 |
| 1068 | LEU131 | C | 47.848 | 16.778 | 21.146 |
| 1069 | LEU131 | O | 46.821 | 17.118 | 21.752 |
| 1070 | CYS132 | N | 48.499 | 17.589 | 20.327 |
| 1071 | CYS132 | CA | 48.159 | 19.011 | 20.295 |
| 1072 | CYS132 | CB | 49.372 | 19.813 | 19.856 |
| 1073 | CYS132 | SG | 50.526 | 20.07 | 21.215 |
| 1074 | CYS132 | C | 46.941 | 19.328 | 19.438 |
| 1075 | CYS132 | O | 46.283 | 20.339 | 19.701 |
| 1076 | ALA133 | N | 46.502 | 18.385 | 18.622 |
| 1077 | ALA133 | CA | 45.227 | 18.555 | 17.926 |
| 1078 | ALA133 | CB | 45.149 | 17.557 | 16.776 |
| 1079 | ALA133 | C | 44.07 | 18.318 | 18.892 |
| 1080 | ALA133 | O | 43.158 | 19.151 | 18.96 |
| 1081 | ARG134 | N | 44.256 | 17.384 | 19.813 |
| 1082 | ARG134 | CA | 43.234 | 17.123 | 20.831 |
| 1083 | ARG134 | CB | 43.594 | 15.848 | 21.581 |
| 1084 | ARG134 | CG | 43.635 | 14.641 | 20.655 |
| 1085 | ARG134 | CD | 44.109 | 13.399 | 21.402 |
| 1086 | ARG134 | NE | 44.245 | 12.259 | 20.483 |
| 1087 | ARG134 | CZ | 43.437 | 11.197 | 20.5 |
| 1088 | ARG134 | NH1 | 42.456 | 11.117 | 21.402 |
| 1089 | ARG134 | NH2 | 43.623 | 10.205 | 19.627 |
| 1090 | ARG134 | C | 43.159 | 18.267 | 21.831 |
| 1091 | ARG134 | O | 42.072 | 18.822 | 22.039 |
| 1092 | PHE135 | N | 44.313 | 18.791 | 22.214 |
| 1093 | PHE135 | CA | 44.322 | 19.9 | 23.171 |
| 1094 | PHE135 | CB | 45.685 | 19.98 | 23.843 |
| 1095 | PHE135 | CG | 45.901 | 18.877 | 24.874 |
| 1096 | PHE135 | CD1 | 47.119 | 18.216 | 24.95 |
| 1097 | PHE135 | CE1 | 47.303 | 17.21 | 25.89 |
| 1098 | PHE135 | CZ | 46.271 | 16.866 | 26.754 |
| 1099 | PHE135 | CE2 | 45.055 | 17.531 | 26.681 |
| 1100 | PHE135 | CD2 | 44.871 | 18.537 | 25.741 |
| 1101 | PHE135 | C | 43.949 | 21.244 | 22.552 |
| 1102 | PHE135 | O | 43.353 | 22.06 | 23.258 |
| 1103 | LEU136 | N | 44.026 | 21.36 | 21.237 |
| 1104 | LEU136 | CA | 43.551 | 22.572 | 20.561 |
| 1105 | LEU136 | CB | 44.343 | 22.767 | 19.273 |
| 1106 | LEU136 | CG | 45.371 | 23.896 | 19.357 |
| 1107 | LEU136 | CD1 | 46.247 | 23.83 | 20.606 |
| 1108 | LEU136 | CD2 | 46.231 | 23.92 | 18.101 |
| 1109 | LEU136 | C | 42.058 | 22.49 | 20.243 |
| 1110 | LEU136 | O | 41.396 | 23.521 | 20.088 |
| 1111 | GLU137 | N | 41.493 | 21.298 | 20.318 |
| 1112 | GLU137 | CA | 40.042 | 21.181 | 20.166 |
| 1113 | GLU137 | CB | 39.693 | 19.859 | 19.493 |
| 1114 | GLU137 | CG | 40.277 | 19.773 | 18.086 |
| 1115 | GLU137 | CD | 39.822 | 20.95 | 17.224 |
| 1116 | GLU137 | OE1 | 40.665 | 21.784 | 16.92 |
| 1117 | GLU137 | OE2 | 38.71 | 20.881 | 16.721 |
| 1118 | GLU137 | C | 39.339 | 21.289 | 21.517 |
| 1119 | GLU137 | O | 38.125 | 21.514 | 21.567 |
| 1120 | VAL138 | N | 40.1 | 21.161 | 22.593 |
| 1121 | VAL138 | CA | 39.558 | 21.424 | 23.929 |
| 1122 | VAL138 | CB | 40.229 | 20.461 | 24.907 |
| 1123 | VAL138 | CG1 | 39.785 | 20.708 | 26.345 |
| 1124 | VAL138 | CG2 | 39.964 | 19.011 | 24.515 |
| 1125 | VAL138 | C | 39.846 | 22.871 | 24.332 |
| 1126 | VAL138 | O | 39.072 | 23.509 | 25.056 |
| 1127 | A5P139 | N | 40.929 | 23.394 | 23.786 |
| 1128 | A5P139 | CA | 41.346 | 24.775 | 24.026 |
| 1129 | A5P139 | CB | 42.022 | 24.83 | 25.398 |
| 1130 | A5P139 | CG | 42.276 | 26.264 | 25.864 |
| 1131 | A5P139 | OD1 | 42.534 | 27.111 | 25.015 |
| 1132 | A5P139 | OD2 | 42.306 | 26.465 | 27.068 |
| 1133 | A5P139 | C | 42.329 | 25.19 | 22.931 |
| 1134 | A5P139 | O | 43.549 | 25.07 | 23.106 |
| 1135 | GLU14O | N | 41.817 | 25.916 | 21.95 |
| 1136 | GLU14O | CA | 42.637 | 26.322 | 20.793 |
| 1137 | GLU14O | CB | 41.728 | 26.643 | 19.611 |
| 1138 | GLU14O | CG | 40.745 | 27.764 | 19.924 |
| 1139 | GLU14O | CD | 39.961 | 28.126 | 18.667 |
| 1140 | GLU14O | OE1 | 38.749 | 28.251 | 18.774 |
| 1141 | GLU14O | OE2 | 40.585 | 28.247 | 17.622 |
| 1142 | GLU14O | C | 43.549 | 27.519 | 21.056 |
| 1143 | GLU14O | O | 44.267 | 27.956 | 20.149 |
| 1144 | ARG141 | N | 43.501 | 28.056 | 22.264 |
| 1145 | ARG141 | CA | 44.365 | 29.164 | 22.649 |
| 1146 | ARG141 | CB | 43.507 | 30.246 | 23.292 |
| 1147 | ARG141 | CG | 42.483 | 30.799 | 22.305 |
| 1148 | ARG141 | CD | 43.158 | 31.518 | 21.14 |
| 1149 | ARG141 | NE | 43.932 | 32.669 | 21.628 |
| 1150 | ARG141 | CZ | 43.547 | 33.936 | 21.459 |
| 1151 | ARG141 | NH1 | 42.481 | 34.215 | 20.703 |
| 1152 | ARG141 | NH2 | 44.276 | 34.926 | 21.978 |
| 1153 | ARG141 | C | 45.454 | 28.699 | 23.613 |
| 1154 | ARG141 | O | 46.132 | 29.54 | 24.217 |
| 1155 | A5N142 | N | 45.558 | 27.393 | 23.824 |
| 1156 | A5N142 | CA | 46.624 | 26.87 | 24.684 |
| 1157 | A5N142 | CB | 46.345 | 25.411 | 25.046 |
| 1158 | A5N142 | CG | 47.367 | 24.918 | 26.074 |
| 1159 | A5N142 | OD1 | 48.138 | 25.713 | 26.627 |
| 1160 | A5N142 | ND2 | 47.424 | 23.611 | 26.254 |
| 1161 | A5N142 | C | 47.965 | 26.985 | 23.968 |
| 1162 | A5N142 | O | 48.385 | 26.074 | 23.241 |
| 1163 | PHE143 | N | 48.734 | 27.963 | 24.42 |
| 1164 | PHE143 | CA | 50.018 | 28.287 | 23.797 |
| 1165 | PHE143 | CB | 50.442 | 29.706 | 24.183 |
| 1166 | PHE143 | CG | 50.738 | 29.965 | 25.664 |
| 1167 | PHE143 | CD1 | 52.031 | 29.809 | 26.147 |
| 1168 | PHE143 | CE1 | 52.309 | 30.05 | 27.486 |
| 1169 | PHE143 | CZ | 51.294 | 30.457 | 28.343 |
| 1170 | PHE143 | CE2 | 50.003 | 30.627 | 27.859 |
| 1171 | PHE143 | CD2 | 49.727 | 30.387 | 26.519 |
| 1172 | PHE143 | C | 51.11 | 27.289 | 24.161 |
| 1173 | PHE143 | O | 52.043 | 27.124 | 23.37 |
| 1174 | HIS144 | N | 50.844 | 26.427 | 25.13 |
| 1175 | H15144 | CA | 51.796 | 25.373 | 25.466 |
| 1176 | H15144 | CB | 51.401 | 24.752 | 26.797 |
| 1177 | H15144 | CG | 51.393 | 25.704 | 27.973 |
| 1178 | H15144 | ND1 | 50.32 | 26.334 | 28.486 |
| 1179 | H15144 | CE1 | 50.706 | 27.099 | 29.527 |
| 1180 | H15144 | NE2 | 52.039 | 26.934 | 29.679 |
| 1181 | H15144 | CO2 | 52.476 | 26.074 | 28.732 |
| 1182 | H15144 | C | 51.787 | 24.286 | 24.4 |
| 1183 | H15144 | O | 52.864 | 23.85 | 23.979 |
| 1184 | CYS145 | N | 50.645 | 24.081 | 23.761 |
| 1185 | CYS145 | CA | 50.595 | 23.08 | 22.695 |
| 1186 | CYS145 | CB | 49.227 | 22.418 | 22.653 |
| 1187 | CYS145 | SG | 49.287 | 20.611 | 22.712 |
| 1188 | CYS145 | C | 50.941 | 23.704 | 21 .346 |
| 1189 | CYS145 | O | 51.488 | 23.012 | 20.48 |
| 1190 | TRP146 | N | 50.884 | 25.024 | 21.271 |
| 1191 | TRP146 | CA | 51.406 | 25.709 | 20.084 |
| 1192 | TRP146 | CD | 50.872 | 27.139 | 20.039 |
| 1193 | TRP146 | CG | 49.412 | 27.26 | 19.648 |
| 1194 | TRP146 | CD1 | 48.326 | 27.378 | 20.487 |
| 1195 | TRP146 | NE1 | 47.202 | 27.46 | 19.73 |
| 1196 | TRP146 | CE2 | 47.497 | 27.407 | 18.418 |
| 1197 | TRP146 | CZ2 | 46.711 | 27.456 | 17.277 |
| 1198 | TRP146 | CH2 | 47.311 | 27.379 | 16.025 |
| 1199 | TRP146 | CZ3 | 48.692 | 27.259 | 15.912 |
| 1200 | TRP146 | CE3 | 49.486 | 27.212 | 17.051 |
| 1201 | TRP146 | CD2 | 48.892 | 27.285 | 18.302 |
| 1202 | TRP146 | C | 52.934 | 25.722 | 20.119 |
| 1203 | TRP146 | O | 53.574 | 25.364 | 19.121 |
| 1204 | ASP147 | N | 53.479 | 25.817 | 21.324 |
| 1205 | ASP147 | CA | 54.927 | 25.731 | 21.528 |
| 1206 | ASP147 | CB | 55.266 | 26.173 | 22.951 |
| 1207 | ASP147 | CG | 54.916 | 27.636 | 23.211 |
| 1208 | ASP147 | 001 | 55.111 | 28.436 | 22.307 |
| 1209 | ASP147 | 002 | 54.614 | 27.948 | 24.357 |
| 1210 | ASP147 | C | 55.424 | 24.301 | 21.364 |
| 1211 | ASP147 | O | 56.499 | 24.094 | 20.79 |
| 1212 | TYR148 | N | 54.572 | 23.332 | 21.655 |
| 1213 | TYR148 | CA | 54.969 | 21.938 | 21.479 |
| 1214 | TYR148 | CB | 54.103 | 21.05 | 22.361 |
| 1215 | TYR148 | CG | 54.695 | 19.657 | 22.55 |
| 1216 | TYR148 | CO1 | 55.754 | 19.493 | 23.433 |
| 1217 | TYR148 | CE1 | 56.32 | 18.239 | 23.614 |
| 1218 | TYR148 | CZ | 55.826 | 17.153 | 22.909 |
| 1219 | TYR148 | OH | 56.436 | 15.929 | 23.048 |
| 1220 | TYR148 | CE2 | 54.764 | 17.31 | 22.028 |
| 1221 | TYR148 | CD2 | 54.198 | 18.566 | 21.847 |
| 1222 | TYR148 | C | 54.85 | 21.503 | 20.023 |
| 1223 | TYR148 | O | 55.678 | 20.707 | 19.569 |
| 1224 | ARG149 | N | 54.03 | 22.193 | 19.246 |
| 1225 | ARG149 | CA | 53.995 | 21.917 | 17.81 |
| 1226 | ARG149 | CB | 52.68 | 22.4 | 17.212 |
| 1227 | ARG149 | CG | 52.637 | 22.043 | 15.732 |
| 1228 | ARG149 | CD | 51.31 | 22.379 | 15.068 |
| 1229 | ARG149 | NE | 51.341 | 21.93 | 13.667 |
| 1230 | ARG149 | CZ | 50.659 | 20.876 | 13.211 |
| 1231 | ARG149 | NH1 | 49.797 | 20.241 | 14.009 |
| 1232 | ARG149 | NH2 | 50.776 | 20.511 | 11.932 |
| 1233 | ARG149 | C | 55.168 | 22.596 | 17.107 |
| 1234 | ARG149 | O | 55.754 | 22.002 | 16.195 |
| 1235 | ARG150 | N | 55.676 | 23.665 | 17.7 |
| 1236 | ARG150 | CA | 56.909 | 24.276 | 17.193 |
| 1237 | ARG150 | CB | 56.989 | 25.71 | 17.706 |
| 1238 | ARG150 | CG | 55.952 | 26.568 | 16.992 |
| 1239 | ARG150 | CD | 56.019 | 28.045 | 17.366 |
| 1240 | ARG150 | NE | 55.239 | 28.349 | 18.575 |
| 1241 | ARG150 | CZ | 54.219 | 29.213 | 18.563 |
| 1242 | ARG150 | NH1 | 53.582 | 29.513 | 19.696 |
| 1243 | ARG15O | NH2 | 53.873 | 29.821 | 17.426 |
| 1244 | ARG150 | C | 58.144 | 23.472 | 17.608 |
| 1245 | ARG150 | O | 59.082 | 23.335 | 16.811 |
| 1246 | PHE151 | N | 58.024 | 22.739 | 18.703 |
| 1247 | PHE151 | CA | 59.073 | 21.804 | 19.112 |
| 1248 | PHE151 | CB | 58.804 | 21.379 | 20.553 |
| 1249 | PHE151 | CG | 59.705 | 20.262 | 21.073 |
| 1250 | PHE151 | CD1 | 61.016 | 20.537 | 21.44 |
| 1251 | PHE151 | CE1 | 61.834 | 19.518 | 21.91 |
| 1252 | PHE151 | CZ | 61.342 | 18.223 | 22.013 |
| 1253 | PHE 151 | CE2 | 60.031 | 17.948 | 21.648 |
| 1254 | PHE 151 | CD2 | 59.213 | 18.967 | 21.179 |
| 1255 | PHE151 | C | 59.091 | 20.578 | 18.205 |
| 1256 | PHE151 | O | 60.165 | 20.192 | 17.729 |
| 1257 | PHE151 | N | 57.92 | 20.133 | 17.778 |
| 1258 | VAL152 | CA | 57.848 | 19.003 | 16.848 |
| 1259 | VAL152 | CB | 56.409 | 18.504 | 16.795 |
| 1260 | VAL152 | CG1 | 56.227 | 17.45 | 15.709 |
| 1261 | VAL152 | CG | 55.966 | 17.963 | 18.148 |
| 1262 | VAL152 | C | 58.296 | 19.409 | 15.448 |
| 1263 | VAL152 | O | 59.078 | 18.678 | 14.829 |
| 1264 | ALA153 | N | 58.051 | 20.658 | 15.087 |
| 1265 | ALA153 | CA | 58.495 | 21.16 | 13.788 |
| 1266 | ALA153 | CB | 57.845 | 22.516 | 13.535 |
| 1267 | ALA153 | C | 60.012 | 21.296 | 13.724 |
| 1268 | ALA153 | O | 60.619 | 20.786 | 12.773 |
| 1269 | THR154 | N | 60.627 | 21.713 | 14.817 |
| 1270 | THR154 | CA | 62.091 | 21.823 | 14.821 |
| 1271 | THR154 | CB | 62.537 | 22.756 | 15.944 |
| 1272 | THR154 | OG1 | 62.022 | 22.282 | 17.183 |
| 1273 | THR154 | CG2 | 62.02 | 24.173 | 15.731 |
| 1274 | THR154 | C | 62.781 | 20.463 | 14.959 |
| 1275 | THR154 | O | 63.717 | 20.197 | 14.196 |
| 1276 | GLN155 | N | 62.148 | 19.534 | 15.659 |
| 1277 | GLN155 | CA | 62.73 | 18.199 | 15.855 |
| 1278 | GLN155 | CB | 62.137 | 17.62 | 17.13 |
| 1279 | GLN155 | CG | 62.64 | 18.292 | 18.399 |
| 1280 | GLN155 | CD | 64.077 | 17.875 | 18.689 |
| 1281 | GLN155 | OE1 | 64.975 | 18.722 | 18.756 |
| 1282 | GLN155 | NE | 64.261 | 16.588 | 18.934 |
| 1283 | GLN155 | C | 62.459 | 17.229 | 14.701 |
| 1284 | GLN155 | O | 62.994 | 16.113 | 14.693 |
| 1285 | ALA156 | N | 61.582 | 17.612 | 13.789 |
| 1286 | ALA156 | CA | 61.358 | 16.827 | 12.574 |
| 1287 | ALA156 | CB | 59.859 | 16.628 | 12.387 |
| 1288 | ALA156 | C | 61.935 | 17.514 | 11.339 |
| 1289 | ALA156 | O | 61.86 | 16.958 | 10.236 |
| 1290 | ALA157 | N | 62.508 | 18.694 | 11.544 |
| 1291 | ALA157 | GA | 63.024 | 19.542 | 10.457 |
| 1292 | ALA157 | CB | 64.214 | 18.863 | 9.782 |
| 1293 | ALA157 | C | 61.937 | 19.866 | 9.435 |
| 1294 | ALA157 | O | 62.094 | 19.625 | 8.232 |
| 1295 | VAL158 | N | 60.844 | 20.42 | 9.932 |
| 1296 | VAL158 | CA | 59.705 | 20.785 | 9.087 |
| 1297 | VAL158 | CB | 58.446 | 20.761 | 9.954 |
| 1298 | VAL158 | OG1 | 57.221 | 21.297 | 9.221 |
| 1299 | VAL158 | CG2 | 58.182 | 19.358 | 10.482 |
| 1300 | VAL158 | C | 59.91 | 22.172 | 8.489 |
| 1301 | VAL158 | O | 60.086 | 23.157 | 9.218 |
| 1302 | PRO159 | N | 59.887 | 22.238 | 7.168 |
| 1303 | PRO159 | CA | 60.044 | 23.514 | 6.469 |
| 1304 | PRO159 | CB | 59.999 | 23.171 | 5.011 |
| 1305 | PRO159 | GG | 59.775 | 21.675 | 4.848 |
| 1306 | PRO159 | CD | 59.7 | 21.107 | 6.254 |
| 1307 | PRO159 | C | 58.938 | 24.497 | 6.839 |
| 1308 | PRO159 | O | 57.754 | 24.136 | 6.907 |
| 1309 | PRO160 | N | 59.312 | 25.762 | 6.955 |
| 1310 | PRO160 | CA | 58.363 | 26.806 | 7.37 |
| 1311 | PRO160 | CB | 59.205 | 28.025 | 7.601 |
| 1312 | PRO160 | CG | 60.643 | 27.732 | 7.2 |
| 1313 | PRO160 | CD | 60.674 | 26.274 | 6.774 |
| 1314 | PRO160 | C | 57.262 | 27.096 | 6.341 |
| 1315 | PRO160 | O | 56.157 | 27.473 | 6.741 |
| 1316 | ALA161 | N | 57.462 | 26.696 | 5.092 |
| 1317 | ALA161 | CA | 56.412 | 26.85 | 4.078 |
| 1318 | ALA161 | CB | 57.061 | 26.833 | 2.699 |
| 1319 | ALA161 | C | 55.355 | 25.746 | 4.166 |
| 1320 | ALA161 | O | 54.177 | 26.009 | 3.902 |
| 1321 | GLU162 | N | 55.707 | 24.64 | 4.803 |
| 1322 | GLU162 | CA | 54.748 | 23.555 | 5.02 |
| 1323 | GLU162 | CB | 55.531 | 22.258 | 5.187 |
| 1324 | GLU162 | CG | 54.62 | 21.064 | 5.447 |
| 1325 | GLU162 | CD | 55.472 | 19.82 | 5.671 |
| 1326 | GLU162 | OE1 | 56.613 | 19.988 | 6.081 |
| 1327 | GLU162 | OE2 | 54.996 | 18.734 | 5.371 |
| 1328 | GLU162 | C | 53.947 | 23.847 | 6.284 |
| 1329 | GLU162 | O | 52.74 | 23.582 | 6.348 |
| 1330 | GLU163 | N | 54.557 | 24.648 | 7.14 |
| 1331 | GLU163 | CA | 53.888 | 25.114 | 8.348 |
| 1332 | GLU163 | CB | 54.973 | 25.598 | 9.297 |
| 1333 | GLU163 | CG | 54.478 | 25.655 | 10.731 |
| 1334 | GLU163 | CD | 54.331 | 24.239 | 11.277 |
| 1335 | GLU163 | OE1 | 55.103 | 23.391 | 10.852 |
| 1336 | GLU163 | OE2 | 53.552 | 24.066 | 12.204 |
| 1337 | GLU163 | C | 52.95 | 26.274 | 8.011 |
| 1338 | GLU163 | O | 51.863 | 26.389 | 8.591 |
| 1339 | LEU164 | N | 53.272 | 26.974 | 6.935 |
| 1340 | LEU164 | CA | 52.412 | 28.042 | 6.435 |
| 1341 | LEU164 | CB | 53.251 | 28.944 | 5.538 |
| 1342 | LEU164 | CG | 52.483 | 30.186 | 5.107 |
| 1343 | LEU164 | CD1 | 52.085 | 31.02 | 6.319 |
| 1344 | LEU164 | CD2 | 53.31 | 31.019 | 4.134 |
| 1345 | LEU164 | C | 51.238 | 27.466 | 5.648 |
| 1346 | LEU164 | O | 50.121 | 27.979 | 5.775 |
| 1347 | ALA165 | N | 51.409 | 26.269 | 5.111 |
| 1348 | ALA165 | CA | 50.288 | 25.578 | 4.465 |
| 1349 | ALA165 | CB | 50.835 | 24.421 | 3.637 |
| 1350 | ALA165 | C | 49.296 | 25.053 | 5.503 |
| 1351 | ALA165 | O | 48.079 | 25.203 | 5.317 |
| 1352 | PHE166 | N | 49.81 | 24.741 | 6.683 |
| 1353 | PHE166 | CA | 48.945 | 24.352 | 7.798 |
| 1354 | PHE166 | CB | 49.809 | 23.777 | 8.915 |
| 1355 | PHE166 | CG | 49.04 | 23.487 | 10.2 |
| 1356 | PHE166 | CD1 | 48.052 | 22.512 | 10.216 |
| 1357 | PHE166 | CE1 | 47.348 | 22.255 | 11.385 |
| 1358 | PHE166 | CZ | 47.632 | 22.974 | 12.539 |
| 1359 | PHE166 | CE2 | 48.62 | 23.95 | 12.523 |
| 1360 | PHE166 | CD2 | 49.324 | 24.207 | 11.354 |
| 1361 | PHE166 | C | 48.153 | 25.545 | 8.329 |
| 1362 | PHE166 | O | 46.93 | 25.44 | 8.475 |
| 1363 | THR167 | N | 48.767 | 26.717 | 8.35 |
| 1364 | THR167 | CA | 48.031 | 27.903 | 8.801 |
| 1365 | THR167 | CB | 49.009 | 28.978 | 9.261 |
| 1366 | THR167 | OG1 | 49.822 | 29.369 | 8.167 |
| 1367 | THR167 | CG2 | 49.915 | 28.476 | 10.38 |
| 1368 | THR167 | C | 47.093 | 28.45 | 7.722 |
| 1369 | THR167 | O | 46.034 | 28.985 | 8.069 |
| 1370 | ASP16B | N | 47.324 | 28.066 | 6.474 |
| 1371 | ASP168 | CA | 46.403 | 28.41 | 5.386 |
| 1372 | ASP168 | CB | 47.027 | 28.033 | 4.042 |
| 1373 | ASP168 | CG | 48.284 | 28.841 | 3.731 |
| 1374 | ASP168 | OD1 | 49.134 | 28.313 | 3.023 |
| 1375 | ASP168 | OD2 | 48.321 | 30.008 | 4.094 |
| 1376 | ASP168 | C | 45.096 | 27.635 | 5.528 |
| 1377 | A3P168 | O | 44.02 | 28.244 | 5.475 |
| 1378 | SER169 | N | 45.19 | 26.39 | 5.973 |
| 1379 | SER169 | CA | 43.975 | 25.586 | 6.16 |
| 1380 | SER169 | CB | 44.315 | 24.102 | 6.071 |
| 1381 | SER169 | CG | 45.147 | 23.759 | 7.17 |
| 1382 | SER169 | C | 43.286 | 25.888 | 7.493 |
| 1383 | SER169 | O | 42.059 | 25.744 | 7.587 |
| 1384 | LEU170 | N | 43.99 | 26.559 | 8.393 |
| 1385 | LEU170 | CA | 43.356 | 27.006 | 9.636 |
| 1386 | LEU170 | CB | 44.422 | 27.406 | 10.649 |
| 1387 | LEU170 | CG | 45.301 | 26.236 | 11.069 |
| 1388 | LEU170 | CD1 | 46.375 | 26.708 | 12.039 |
| 1389 | LEU170 | CD2 | 44.476 | 25.113 | 11.689 |
| 1390 | LEU170 | C | 42.461 | 28.215 | 9.386 |
| 1391 | LEU170 | O | 41.373 | 28.3 | 9.972 |
| 1392 | ILEA171 | N | 42.748 | 28.945 | 8.322 |
| 1393 | ILEA171 | CA | 41.93 | 30.111 | 7.988 |
| 1394 | ILEA171 | CB | 42.806 | 31.078 | 7.191 |
| 1395 | ILEA171 | CG2 | 42.05 | 32.347 | 6.808 |
| 1396 | ILEA171 | CG1 | 44.055 | 31.443 | 7.986 |
| 1397 | ILEA171 | CD1 | 43.711 | 32.088 | 9.325 |
| 1398 | ILEA171 | C | 40.688 | 29.721 | 7.183 |
| 1399 | 1LEA171 | O | 39.694 | 30.457 | 7.199 |
| 1400 | THR172 | N | 40.654 | 28.499 | 6.674 |
| 1401 | THR172 | GA | 39.519 | 28.101 | 5.838 |
| 1402 | THR172 | CB | 40.OD2 | 27.177 | 4.726 |
| 1403 | THR172 | OG1 | 40.422 | 25.949 | 5.302 |
| 1404 | THR172 | CG2 | 41.166 | 27.783 | 3.953 |
| 1405 | THR172 | C | 38.396 | 27.406 | 6.609 |
| 1406 | THR172 | O | 37.29 | 27.311 | 6.066 |
| 1407 | ARG173 | N | 38.646 | 26.949 | 7.83 |
| 1408 | ARG173 | CA | 37.554 | 26.333 | 8.605 |
| 1409 | ARG173 | CB | 37.2 | 24.98 | 7.987 |
| 1410 | ARG173 | CG | 35.777 | 24.56 | 8.349 |
| 1411 | ARG173 | CD | 35.427 | 23.175 | 7.816 |
| 1412 | ARG173 | NE | 34.053 | 22.808 | 8.199 |
| 1413 | ARG173 | VZ | 33.763 | 21.959 | 9.187 |
| 1414 | ARG173 | NH1 | 34.745 | 21.361 | 9.865 |
| 1415 | ARG173 | NH2 | 32.49 | 21 .685 | 9.48 |
| 1416 | ARG173 | C | 37.894 | 26.143 | 10.087 |
| 1417 | ARG173 | O | 37.136 | 25.499 | 10.824 |
| 1418 | ASN174 | N | 39.012 | 26.673 | 10.542 |
| 1419 | ASN174 | CA | 39.328 | 26.506 | 11.962 |
| 1420 | ASN174 | CB | 40.818 | 26.225 | 12.133 |
| 1421 | ASN174 | CG | 41.146 | 25.798 | 13.56 |
| 1422 | ASN174 | OD1 | 42.199 | 26.154 | 14.103 |
| 1423 | ASN174 | ND2 | 40.255 | 25.011 | 14.14 |
| 1424 | ASN174 | C | 38.902 | 27.755 | 12.723 |
| 1425 | ASN174 | O | 37.811 | 27.768 | 13.307 |
| 1426 | PHE175 | N | 39.693 | 28.81 | 12.615 |
| 1427 | PHE175 | CA | 39.389 | 30.049 | 13.338 |
| 1428 | PHE175 | CB | 39.488 | 29.769 | 14.839 |
| 1429 | PHE175 | CG | 38.631 | 30.676 | 15.719 |
| 1430 | PHE175 | CD1 | 37.307 | 30.913 | 15.375 |
| 1431 | PHE175 | CE1 | 36.519 | 31.735 | 16.171 |
| 1432 | PHE175 | CZ | 37.056 | 32.317 | 17.311 |
| 1433 | PHE175 | CE2 | 38.38 | 32.079 | 17.656 |
| 1434 | PHE175 | OD2 | 39.168 | 31.257 | 16.86 |
| 1435 | PHE175 | C | 40.397 | 31.131 | 12.963 |
| 1436 | PHE175 | O | 41.432 | 30.837 | 12.352 |
| 1437 | SER176 | N | 40.043 | 32.376 | 13.245 |
| 1438 | SER176 | CA | 41.016 | 33.472 | 13.148 |
| 1439 | SER176 | CB | 40.335 | 34.823 | 13.39 |
| 1440 | SER176 | OG | 39.504 | 34.778 | 14.544 |
| 1441 | SER176 | C | 42.174 | 33.171 | 14.111 |
| 1442 | SER176 | O | 43.208 | 32.702 | 13.626 |
| 1443 | ASN177 | N | 42.096 | 33.622 | 15.358 |
| 1444 | ASN177 | CA | 42.903 | 33.035 | 16.444 |
| 1445 | ASN177 | CB | 43.037 | 31.518 | 16.252 |
| 1446 | ASN177 | CG | 43.77 | 30.824 | 17.401 |
| 1447 | ASN177 | OD1 | 44.69 | 31.383 | 18.009 |
| 1448 | ASN177 | ND2 | 43.378 | 29.591 | 17.663 |
| 1449 | ASN177 | C | 44.252 | 33.739 | 16.496 |
| 1450 | ASN177 | O | 45.111 | 33.532 | 15.634 |
| 1451 | TYR178 | N | 44.509 | 34.384 | 17.62 |
| 1452 | TYR178 | CA | 45.681 | 35.254 | 17.732 |
| 1453 | TYR178 | CB | 45.447 | 36.185 | 18.914 |
| 1454 | TYR178 | CG | 46.53 | 37.232 | 19.138 |
| 1455 | TYR178 | CD1 | 46.609 | 38.334 | 18.297 |
| 1456 | TYR178 | CE1 | 47.594 | 39.292 | 18.499 |
| 1457 | TYR178 | CZ | 48.496 | 39.143 | 19.545 |
| 1458 | TYR178 | OH | 49.463 | 40.099 | 19.756 |
| 1459 | TYR178 | CE2 | 48.419 | 38.042 | 20.388 |
| 1460 | TYR178 | CD2 | 47.434 | 37.085 | 20.184 |
| 1461 | TYR178 | C | 46.995 | 34.492 | 17.9 |
| 1462 | TYR178 | O | 48.028 | 34.994 | 17.446 |
| 1463 | SER179 | N | 46.938 | 33.225 | 18.275 |
| 1464 | SER179 | CA | 48.179 | 32.453 | 18.365 |
| 1465 | SER179 | CB | 48.079 | 31.413 | 19.475 |
| 1466 | SER179 | OG | 47.051 | 30.494 | 19.143 |
| 1467 | SER179 | C | 48.497 | 31.79 | 17.024 |
| 1468 | SER179 | O | 49.675 | 31.598 | 16.701 |
| 1469 | SER180 | N | 47.5 | 31.677 | 16.158 |
| 1470 | SER180 | CA | 47.78 | 31.182 | 14.807 |
| 1471 | SER180 | CB | 46.608 | 30.373 | 14.261 |
| 1472 | SER180 | OG | 45.499 | 31.234 | 14.081 |
| 1473 | SER180 | C | 48.11 | 32.353 | 13.883 |
| 1474 | SER180 | O | 48.948 | 32.201 | 12.987 |
| 1475 | TRP181 | N | 47.678 | 33.546 | 14.266 |
| 1476 | TRP181 | CA | 48.131 | 34.762 | 13.583 |
| 1477 | TRP181 | CB | 47.196 | 35.919 | 13.912 |
| 1478 | TRP181 | CG | 45.851 | 35.935 | 13.205 |
| 1479 | TRP181 | CD1 | 44.638 | 36.22 | 13.79 |
| 1480 | TRP181 | NE1 | 43.678 | 36.186 | 12.834 |
| 1481 | TRP181 | CE2 | 44.198 | 35.884 | 11.632 |
| 1482 | TRP181 | CZ2 | 43.638 | 35.777 | 10.367 |
| 1483 | TRP181 | CH2 | 44.444 | 35.458 | 9.28 |
| 1484 | TRP181 | CZ3 | 45.805 | 35.244 | 9.457 |
| 1485 | TRP181 | CE3 | 46.376 | 35.353 | 10.72 |
| 1486 | TRP181 | CD2 | 45.579 | 35.679 | 11.808 |
| 1487 | TRP181 | C | 49.547 | 35.129 | 14.02 |
| 1488 | TRP181 | O | 50.341 | 35.599 | 13.198 |
| 1489 | HIS182 | N | 49.917 | 34.711 | 15.22 |
| 1490 | HIS182 | CA | 51.3 | 34.84 | 15.683 |
| 1491 | HIS182 | CB | 51.305 | 34.599 | 17.188 |
| 1492 | HIS182 | CG | 52.675 | 34.403 | 17.806 |
| 1493 | HIS182 | ND1 | 53.777 | 35.149 | 17.596 |
| 1494 | HIS182 | CE1 | 54.794 | 34.652 | 18.331 |
| 1495 | HIS182 | NE2 | 54.327 | 33.576 | 19.005 |
| 1496 | HIS182 | CD2 | 53.023 | 33.411 | 18.692 |
| 1497 | HIS182 | C | 52.21 | 33.828 | 14.994 |
| 1498 | HIS182 | O | 53.326 | 34.183 | 14.594 |
| 1499 | TYR183 | N | 51.661 | 32.68 | 14.637 |
| 1500 | TYR183 | CA | 52.452 | 31.706 | 13.894 |
| 1501 | TYR183 | CB | 51.724 | 30.369 | 13.925 |
| 1502 | TYR183 | CG | 52.649 | 29.157 | 13.914 |
| 1503 | TYR183 | CD1 | 54.002 | 29.309 | 13.636 |
| 1504 | TYR183 | CE1 | 54.842 | 28.203 | 13.641 |
| 1505 | TYR183 | CZ | 54.324 | 26.947 | 13.933 |
| 1506 | TYR183 | OH | 55.156 | 25.847 | 13.943 |
| 1507 | TYR183 | CE2 | 52.976 | 26.793 | 14.221 |
| 1508 | TYR183 | CD2 | 52.138 | 27.9 | 14.214 |
| 1509 | TYR183 | C | 52.645 | 32.18 | 12.454 |
| 1510 | TYR183 | O | 53.784 | 32.165 | 11.968 |
| 1511 | ARG184 | N | 51.654 | 32.867 | 11.906 |
| 1512 | ARG184 | CA | 51.812 | 33.424 | 10.558 |
| 1513 | ARG184 | CB | 50.45 | 33.758 | 9.972 |
| 1514 | ARG184 | CD | 49.584 | 32.516 | 9.848 |
| 1515 | ARG184 | CD | 48.428 | 32.776 | 8.895 |
| 1516 | ARG184 | NE | 48.966 | 33.118 | 7.57 |
| 1517 | ARG184 | CZ | 48.43 | 32.69 | 6.427 |
| 1518 | ARG184 | NH1 | 47.289 | 32.O01 | 6.445 |
| 1519 | ARG184 | NH2 | 48.999 | 33.01 | 5.264 |
| 1520 | ARG184 | C | 52.675 | 34.682 | 10.538 |
| 1521 | ARG184 | O | 53.419 | 34.874 | 9.572 |
| 1522 | SER185 | N | 52.766 | 35.379 | 11.661 |
| 1523 | SER185 | CA | 53.664 | 36.536 | 11.766 |
| 1524 | SER185 | CB | 53.16 | 37.509 | 12.825 |
| 1525 | SER185 | OG | 53.298 | 36.906 | 14.1 |
| 1526 | SER185 | C | 55.098 | 36.122 | 12.096 |
| 1527 | SER185 | O | 55.95 | 36.99 | 12.311 |
| 1528 | CYS186 | N | 55.336 | 34.828 | 12.236 |
| 1529 | CYS186 | CA | 56.701 | 34.315 | 12.241 |
| 1530 | CYS186 | CB | 56.815 | 33.2 | 13.274 |
| 1531 | GY5186 | SG | 56.497 | 33.68 | 14.987 |
| 1532 | CY3186 | C | 57.028 | 33.764 | 10.856 |
| 1533 | CYS186 | O | 57.937 | 34.281 | 10.19 |
| 1534 | LEU187 | N | 56.113 | 32.961 | 10.335 |
| 1535 | LEU 187 | CA | 56.332 | 32.255 | 9.061 |
| 1536 | LEU187 | CB | 55.159 | 31.312 | 8.82 |
| 1537 | LEU187 | CG | 55.082 | 30.226 | 9.885 |
| 1538 | LEU187 | CD1 | 53.774 | 29.451 | 9.781 |
| 1539 | LEU187 | CD2 | 56.281 | 29.289 | 9.814 |
| 1540 | LEU187 | C | 56.465 | 33.188 | 7.865 |
| 1541 | LEU187 | O | 57.463 | 33.105 | 7.138 |
| 1542 | LEU188 | N | 55.605 | 34.189 | 7.78 |
| 1543 | LEU188 | CA | 55.699 | 35.159 | 6.677 |
| 1544 | LEU188 | CB | 54.488 | 36.087 | 6.694 |
| 1545 | LEU188 | CC | 53.19 | 35.313 | 6.489 |
| 1546 | LEU188 | CD1 | 51.984 | 36.192 | 6.772 |
| 1547 | LEU188 | CD2 | 53.102 | 34.709 | 5.094 |
| 1548 | LEU188 | G | 57.024 | 35.945 | 6.684 |
| 1549 | LEU188 | O | 57.732 | 35.831 | 5.675 |
| 1550 | PRO189 | N | 57.439 | 36.622 | 7.757 |
| 1551 | PRO189 | CA | 58.778 | 37.238 | 7.745 |
| 1552 | PRO189 | CB | 58.861 | 38.065 | 8.988 |
| 1553 | PRO189 | CG | 57.604 | 37.867 | 9.809 |
| 1554 | PRO189 | CD | 56.732 | 36.914 | 9.015 |
| 1555 | PRO189 | C | 59.978 | 36.274 | 7.672 |
| 1556 | PRO189 | O | 61.06 | 36.728 | 7.283 |
| 1557 | GLN190 | N | 59.793 | 34.982 | 7.894 |
| 1558 | GLN190 | CA | 60.892 | 34.031 | 7.692 |
| 1559 | GLN190 | CB | 60.682 | 32.845 | 8.626 |
| 1560 | GLN190 | CG | 60.77 | 33.257 | 10.089 |
| 1561 | GLN190 | GD | 60.446 | 32.066 | 10.986 |
| 1562 | GLN190 | OE1 | 59.278 | 31.708 | 11.192 |
| 1563 | GLN190 | NE2 | 61.496 | 31.47 | 11.521 |
| 1564 | GLN190 | C | 60.967 | 33.509 | 6.257 |
| 1565 | GLN190 | O | 61.983 | 32.913 | 5.88 |
| 1566 | LEU191 | N | 59.931 | 33.738 | 5.466 |
| 1567 | LEU191 | CA | 59.911 | 33.21 | 64.095 |
| 1568 | LEU191 | CB | 58.644 | 32.38 | 3.936 |
| 1569 | LEU191 | CG | 58.635 | 31.14 | 94.833 |
| 1570 | LEU191 | CD1 | 57.247 | 30.52 | 4.874 |
| 1571 | LEU191 | CD2 | 59.685 | 30.13 | 84.388 |
| 1572 | LEU191 | C | 59.885 | 34.29 | 3.01 |
| 1573 | LEU1Y1 | O | 60.181 | 33.98 | 71.847 |
| 1574 | HIS192 | N | 59.477 | 35.50 | 13.346 |
| 1575 | HIS192 | CA | 59.23 | 36.48 | 72.278 |
| 1576 | HIS192 | CB | 57.736 | 36.80 | 72.239 |
| 1577 | HIS192 | CG | 56.856 | 35.60 | 41.966 |
| 1578 | HIS192 | ND1 | 57.049 | 34.66 | 11.023 |
| 1579 | HIS192 | CE1 | 56.055 | 33.75 | 31.091 |
| 1580 | HIS192 | NE2 | 55.228 | 34.12 | 62.093 |
| 1581 | HIS192 | CD2 | 55.709 | 35.26 | 52.642 |
| 1582 | HIS192 | C | 60.071 | 37.77 | 82.287 |
| 1583 | HIS192 | O | 60.721 | 38.02 | 21.264 |
| 1584 | PRO193 | N | 60.006 | 38.64 | 3.301 |
| 1585 | PRO193 | CA | 60.485 | 40.01 | 83.097 |
| 1586 | PRO193 | CB | 60.03 | 40.798 | 4.29 |
| 1587 | PRO193 | CG | 59.33 | 39.868 | 5.26 |
| 1588 | PRO193 | CD | 59.308 | 38.50 | 94.586 |
| 1589 | PRO193 | C | 61.995 | 40.14 | 12.945 |
| 1590 | PRO193 | O | 62.765 | 39.78 | 43.842 |
| 1591 | GLN194 | N | 62.391 | 40.667 | 1.8 |
| 1592 | GLN194 | CA | 63.785 | 41.05 | 81.582 |
| 1593 | GLN194 | CB | 64.203 | 40.60 | 60.185 |
| 1594 | GLN194 | OG | 63.131 | 40.924 | −0.853 |
| 1595 | GLN194 | CD | 63.603 | 40.51 | −2.241 |
| 1596 | GLN194 | OE1 | 63.764 | 39.319 | −2.532 |
| 1597 | GLN194 | NE2 | 63.819 | 41.505 | −3.083 |
| 1598 | GLN194 | C | 63.936 | 42.57 | 11.756 |
| 1599 | GLN194 | O | 63.465 | 43.36 | 30.929 |
| 1600 | PRO195 | N | 64.527 | 42.95 | 72.876 |
| 1601 | PRO195 | CA | 64.609 | 44.37 | 33.243 |
| 1602 | PRO195 | CB | 65.082 | 44.38 | 74.663 |
| 1603 | PRO195 | CG | 65.422 | 42.96 | 65.091 |
| 1604 | PRO195 | CD | 65.082 | 42.07 | 73.907 |
| 1605 | PRO195 | C | 65.569 | 45.13 | 42.337 |
| 1606 | PRO195 | O | 66.778 | 44.88 | 12.322 |
| 1607 | ASP196 | N | 65.009 | 46.04 | 71.565 |
| 1608 | ASP196 | CA | 65.821 | 46.87 | 50.675 |
| 1609 | ASP196 | CB | 65.139 | 46.901 | −0.693 |
| 1610 | ASP196 | CG | 66.095 | 47.35 | −1.797 |
| 1611 | ASP196 | OD1 | 65.967 | 48.504 | −2.189 |
| 1612 | ASP196 | OD2 | 66.832 | 46.518 | −2.303 |
| 1613 | ASP196 | C | 65.983 | 48.26 | 41.305 |
| 1614 | ASP196 | O | 66.663 | 48.385 | 2.33 |
| 1615 | SER197 | N | 65.392 | 49.28 | 90.711 |
| 1616 | SER197 | CA | 65.491 | 50.64 | 1.273 |
| 1617 | SER197 | CB | 66.804 | 51.25 | 80.804 |
| 1618 | SER197 | OG | 66.894 | 52.56 | 51.357 |
| 1619 | SER197 | C | 64.326 | 51.51 | 90.825 |
| 1620 | SER197 | O | 64.006 | 52.526 | 1.469 |
| 1621 | GLY198 | N | 63.706 | 51.128 | −0.276 |
| 1622 | GLY198 | CA | 62.587 | 51.892 | −0.847 |
| 1623 | GLY198 | C | 61.318 | 51.828 | 0.002 |
| 1624 | GLY198 | O | 61.172 | 52.578 | 0.975 |
| 1625 | PRO199 | N | 60.392 | 50.981 | −0.419 |
| 1626 | PRO199 | CA | 59.086 | 50.871 | 0.24 |
| 1627 | PRO199 | CB | 58.296 | 49.916 | −0.601 |
| 1628 | PRO199 | CG | 59.169 | 49.406 | −1.738 |
| 1629 | PRO199 | CD | 60.507 | 50.11 | −1.591 |
| 1630 | PRO199 | C | 59.209 | 50.368 | 1.674 |
| 1631 | PRO199 | O | 60.011 | 49.477 | 1.974 |
| 1632 | GLN200 | N | 58.381 | 50.932 | 2.537 |
| 1633 | GLN200 | CA | 58.395 | 50.591 | 3.965 |
| 1634 | GLN200 | CB | 58.256 | 51.903 | 4.724 |
| 1635 | GLN200 | OG | 58.723 | 51.821 | 6.17 |
| 1636 | GLN200 | CD | 58.63 | 53.214 | 6.769 |
| 1637 | GLN200 | OE1 | 57.586 | 53.877 | 6.685 |
| 1638 | GLN200 | NE2 | 59.743 | 53.657 | 7.324 |
| 1639 | GLN200 | C | 57.282 | 49.611 | 4.375 |
| 1640 | GLN200 | O | 56.898 | 49.571 | 5.549 |
| 1641 | GLY201 | N | 56.766 | 48.839 | 3.432 |
| 1642 | GLY201 | CA | 55.678 | 47.894 | 3.741 |
| 1643 | GLY201 | C | 56.143 | 46.831 | 4.733 |
| 1644 | GLY201 | O | 57.35 | 46.602 | 4.872 |
| 1645 | ARG202 | N | 55.213 | 46.298 | 5.508 |
| 1646 | ARG202 | CA | 55.569 | 45.263 | 6.485 |
| 1647 | ARG202 | CB | 54.336 | 44.894 | 7.3 |
| 1648 | ARG202 | CG | 54.753 | 44.296 | 8.636 |
| 1649 | ARG202 | CD | 55.572 | 45.324 | 9.405 |
| 1650 | ARG202 | NE | 56.039 | 44.812 | 10.701 |
| 1651 | ARG202 | CZ | 55.731 | 45.41 | 1.859 |
| 1652 | ARG202 | NH1 | 54.857 | 46.407 | 11.883 |
| 1653 | ARG202 | NH2 | 56.229 | 44.923 | 13.002 |
| 1654 | ARG202 | C | 56.085 | 44.036 | 5.742 |
| 1655 | ARG202 | O | 57.276 | 43.706 | 5.794 |
| 1656 | LEU203 | N | 55.183 | 43.393 | 5.025 |
| 1657 | LEU203 | CA | 55.57 | 42.332 | 4.094 |
| 1658 | LEU203 | CB | 54.458 | 41.288 | 4.045 |
| 1659 | LEU203 | CG | 54.283 | 40.571 | 5.377 |
| 1660 | LEU203 | CD1 | 53.088 | 39.627 | 5.32 |
| 1661 | LEU203 | CD2 | 55.547 | 39.811 | 5.764 |
| 1662 | LEU203 | C | 55.774 | 42.959 | 2.717 |
| 1663 | LEU203 | O | 55.332 | 44.094 | 2.498 |
| 1664 | PRO204 | N | 56.453 | 42.26 | 1.816 |
| 1665 | PRO204 | CA | 56.416 | 42.65 | 0.405 |
| 1666 | PRO204 | CB | 57.184 | 41.598 | −0.331 |
| 1667 | PRO204 | CG | 57.659 | 40.546 | 0.659 |
| 1668 | PRO204 | CD | 57.145 | 40.985 | 2.021 |
| 1669 | PRO204 | C | 54.963 | 42.715 | −0.04 |
| 1670 | PRO204 | O | 54.164 | 41.847 | 0.332 |
| 1671 | GLU205 | N | 54.649 | 43.632 | −0.94 |
| 1672 | GLU205 | CA | 53.236 | 43.949 | −1.207 |
| 1673 | GLU205 | CB | 53.168 | 45.225 | −2.039 |
| 1674 | GLU205 | CG | 51.748 | 45.779 | −2.046 |
| 1675 | GLU205 | CD | 51.635 | 47.007 | −2.94 |
| 1676 | GLU205 | OE1 | 52.117 | 48.057 | −2.536 |
| 1677 | GLU205 | OE2 | 51.076 | 46.876 | −4.02 |
| 1678 | GLU205 | C | 52.452 | 42.833 | −1.908 |
| 1679 | GLU205 | O | 51.26 | 42.686 | −1.621 |
| 1680 | ASP206 | N | 53.147 | 41.887 | −2.522 |
| 1681 | ASP206 | CA | 52.469 | 40.754 | −3.164 |
| 1682 | ASP206 | CB | 53.434 | 40.083 | −4.148 |
| 1683 | ASP206 | GG | 54.714 | 39.593 | −3.465 |
| 1684 | ASP206 | OD1 | 55.618 | 40.404 | −3.302 |
| 1685 | ASP206 | OD2 | 54.748 | 38.436 | −3.073 |
| 1686 | ASP206 | C | 51.942 | 39.725 | −2.154 |
| 1687 | ASP206 | O | 50.943 | 39.058 | −2.44 |
| 1688 | VAL207 | N | 52.485 | 39.709 | −0.945 |
| 1689 | VAL207 | CA | 51.935 | 38.83 | 0.084 |
| 1690 | VAL207 | CB | 53.048 | 37.972 | 0.694 |
| 1691 | VAL207 | CG1 | 54.289 | 38.775 | 1.057 |
| 1692 | VAL207 | CG2 | 52.559 | 37.162 | 1.89 |
| 1693 | VAL207 | C | 51.209 | 39.665 | 1.133 |
| 1694 | VAL207 | O | 50.206 | 39.219 | 1.703 |
| 1695 | LEU208 | N | 51.519 | 40.95 | 1.147 |
| 1696 | LEU208 | CA | 50.912 | 41.852 | 2.118 |
| 1697 | LEU208 | CB | 51.742 | 43.128 | 2.16 |
| 1698 | LEU208 | CG | 51.301 | 44.037 | 3.296 |
| 1699 | LEU208 | CD1 | 51.351 | 43.287 | 4.62 |
| 1700 | LEU208 | CD2 | 52.168 | 45.287 | 3.352 |
| 1701 | LEU208 | C | 49.474 | 42.189 | 1.752 |
| 1702 | LEU208 | O | 48.614 | 42.131 | 2.638 |
| 1703 | LEU209 | N | 49.163 | 42.223 | 0.465 |
| 1704 | LEU209 | CA | 47.787 | 42.54 | 0.069 |
| 1705 | LEU209 | CB | 47.731 | 42.945 | −1.4 |
| 1706 | LEU209 | CG | 48.528 | 44.212 | −1.68 |
| 1707 | LEU209 | CD1 | 48.351 | 44.644 | −3.131 |
| 1708 | LEU209 | CD2 | 48.131 | 45.341 | −0.737 |
| 1709 | LEU209 | C | 46.853 | 41.359 | 0.29 |
| 1710 | LEU209 | O | 45.751 | 41.562 | 0.817 |
| 1711 | LYS210 | N | 47.375 | 40.148 | 0.177 |
| 1712 | LYS210 | CA | 46.521 | 38.991 | 0.436 |
| 1713 | LYS210 | CB | 46.984 | 37.78 | −0.373 |
| 1714 | LYS210 | CB | 48.387 | 37.307 | −0.018 |
| 1715 | LYS210 | CD | 48.792 | 36.106 | −0.863 |
| 1716 | LYS210 | CE | 50.17 | 35.59 | −0.469 |
| 1717 | LYS210 | NZ | 50.565 | 34.443 | −1.301 |
| 1718 | LYS210 | C | 46.451 | 38.683 | 1.93 |
| 1719 | LYS210 | O | 45.401 | 38.223 | 2.385 |
| 1720 | GLU211 | N | 47.388 | 39.204 | 2.708 |
| 1721 | GLU211 | CA | 47.286 | 39.077 | 4.163 |
| 1722 | GLU211 | CB | 48.653 | 39.288 | 4.793 |
| 1723 | GLU211 | CG | 49.591 | 38.128 | 4.506 |
| 1724 | GLU211 | CD | 48.954 | 36.827 | 4.974 |
| 1725 | GLU211 | OE1 | 48.749 | 35.975 | 4.122 |
| 1726 | GLU211 | OE2 | 48.813 | 36.661 | 6.178 |
| 1727 | GLU211 | C | 46.311 | 40.096 | 4.732 |
| 1728 | GLU211 | O | 45.496 | 39.74 | 5.594 |
| 1729 | LEU212 | N | 46.22 | 41.241 | 4.073 |
| 1730 | LEU212 | CA | 45.237 | 42.256 | 4.451 |
| 1731 | LEU212 | CB | 45.526 | 43.533 | 3.669 |
| 1732 | LEU212 | CG | 46.782 | 44.242 | 4.16 |
| 1733 | LEU212 | CD1 | 47.221 | 45.323 | 3.181 |
| 1734 | LEU212 | CD2 | 46.572 | 44.823 | 5.552 |
| 1735 | LEU212 | C | 43.828 | 41.779 | 4.133 |
| 1736 | LEU212 | O | 42.959 | 41.86 | 5.007 |
| 1737 | GLU213 | N | 43.702 | 41.006 | 3.065 |
| 1738 | GLU213 | CA | 42.405 | 40.436 | 2.687 |
| 1739 | GLU213 | CB | 42.462 | 40.152 | 1.194 |
| 1740 | GLU213 | CG | 42.651 | 41.457 | 0.429 |
| 1741 | GLU213 | CD | 43.107 | 41.172 | −0.997 |
| 1742 | GLU213 | OE1 | 42.854 | 42.004 | −1.857 |
| 1743 | GLU213 | OE2 | 43.787 | 40.171 | −1.185 |
| 1744 | GLU213 | C | 42.051 | 39.163 | 3.461 |
| 1745 | GLU213 | O | 40.863 | 38.897 | 3.68 |
| 1746 | LEU214 | N | 43.04 | 38.509 | 4.048 |
| 1747 | LEU214 | CA | 42.752 | 37.347 | 4.896 |
| 1748 | LEU214 | CB | 44.014 | 36.521 | 5.121 |
| 1749 | LEU214 | CG | 44.386 | 35.713 | 3.885 |
| 1750 | LEU214 | CO1 | 45.669 | 34.925 | 4.119 |
| 1751 | LEU214 | CD2 | 43.251 | 34.777 | 3.485 |
| 1752 | LEU214 | C | 42.195 | 37.784 | 6.24 |
| 1753 | LEU214 | O | 41.133 | 37.29 | 6.641 |
| 1754 | VAL215 | N | 42.739 | 38.857 | 6.793 |
| 1755 | VAL215 | CA | 42.174 | 39.371 | 8.041 |
| 1756 | VAL215 | CB | 43.223 | 40.157 | 8.817 |
| 1757 | VAL215 | OG1 | 44.223 | 39.223 | 9.478 |
| 1758 | VAL215 | CG2 | 43.942 | 41.175 | 7.947 |
| 1759 | VAL215 | C | 40.932 | 40.216 | 7.778 |
| 1760 | VAL215 | O | 39.994 | 40.149 | 8.582 |
| 1761 | GLN216 | N | 40.798 | 40.707 | 6.555 |
| 1762 | GLN216 | CA | 39.6 | 41.435 | 6.14 |
| 1763 | GLN216 | CB | 39.866 | 42.025 | 4.757 |
| 1764 | GLN216 | CG | 38.704 | 42.861 | 4.241 |
| 1765 | GLN216 | CD | 39.031 | 43.462 | 2.876 |
| 1766 | GLN216 | OE1 | 40.14 | 43.297 | 2.35 |
| 1767 | GLN216 | NE2 | 38.087 | 44.232 | 2.359 |
| 1768 | GLN216 | C | 38.397 | 40.504 | 6.095 |
| 1769 | GLN216 | O | 37.415 | 40.754 | 6.806 |
| 1770 | ASN217 | N | 38.596 | 39.316 | 5.552 |
| 1771 | ASN217 | CA | 37.503 | 38.345 | 5.502 |
| 1772 | ASN217 | CB | 37.813 | 37.294 | 4.441 |
| 1773 | ASN217 | CG | 37.594 | 37.833 | 3.028 |
| 1774 | ASN217 | OD1 | 37.54 | 39.046 | 2.784 |
| 1775 | ASN217 | ND2 | 37.385 | 36.902 | 2.114 |
| 1776 | A3N217 | C | 37.281 | 37.659 | 6.848 |
| 1777 | ASN217 | O | 36.123 | 37.451 | 7.228 |
| 1778 | ALA218 | N | 38.323 | 37.574 | 7.66 |
| 1779 | ALA218 | CA | 38.178 | 36.97 | 8.987 |
| 1780 | ALA218 | CB | 39.564 | 36.74 | 9.579 |
| 1781 | ALA218 | C | 37.349 | 37.848 | 9.921 |
| 1782 | ALA218 | O | 36.333 | 37.373 | 10.449 |
| 1783 | PHE219 | N | 37.587 | 39.15 | 9.893 |
| 1784 | PHE219 | CA | 36.793 | 40.037 | 10.744 |
| 1785 | PHE219 | CB | 37.629 | 41.198 | 11.284 |
| 1786 | PHE219 | CG | 38.335 | 42.163 | 10.326 |
| 1787 | PHE219 | CD1 | 37.643 | 42.816 | 9.314 |
| 1788 | PHE219 | CE1 | 38.307 | 43.706 | 8.478 |
| 1789 | PHE219 | CZ | 39.661 | 43.954 | 8.662 |
| 1790 | PHE219 | CE2 | 40.349 | 43.317 | 9.685 |
| 1791 | PHE219 | CD2 | 39.685 | 42.431 | 10.52 |
| 1792 | PHE219 | C | 35.492 | 40.503 | 10.086 |
| 1793 | PHE219 | O | 34.66 | 41.122 | 10.753 |
| 1794 | PHE220 | N | 35.258 | 40.121 | 8.841 |
| 1795 | PHE220 | CA | 33.926 | 40.327 | 8.262 |
| 1796 | PHE220 | CB | 34.025 | 40.662 | 6.779 |
| 1797 | PHE220 | CG | 34.533 | 42.072 | 6.498 |
| 1798 | PHE220 | CD1 | 35.065 | 42.386 | 5.255 |
| 1799 | PHE220 | CE1 | 35.528 | 43.671 | 5.007 |
| 1800 | PHE220 | CZ | 35.454 | 44.642 | 5.996 |
| 1801 | PHE220 | CE2 | 34.903 | 44.335 | 7.231 |
| 1802 | PHE220 | CD2 | 34.437 | 43.052 | 7.478 |
| 1803 | PHE220 | C | 33.048 | 39.096 | 8.466 |
| 1804 | PHE220 | O | 31.825 | 39.165 | 8.298 |
| 1805 | THR221 | N | 33.666 | 37.996 | 8.867 |
| 1806 | THR221 | CA | 32.906 | 36.812 | 9.266 |
| 1807 | THR221 | CB | 33.75 | 35.575 | 8.972 |
| 1808 | THR221 | OG1 | 34.03 | 35.562 | 7.58 |
| 1809 | THR221 | CG2 | 33.017 | 34.282 | 9.318 |
| 1810 | THR221 | C | 32.601 | 36.901 | 10.758 |
| 1811 | THR221 | O | 31.58 | 36.393 | 11.238 |
| 1812 | ASP222 | N | 33.477 | 37.584 | 11.475 |
| 1813 | ASP222 | CA | 33.202 | 37.911 | 12.878 |
| 1814 | ASP222 | CB | 33.673 | 36.758 | 13.765 |
| 1815 | ASP222 | CG | 33.321 | 36.993 | 15.236 |
| 1816 | ASP222 | OD1 | 32.643 | 37.977 | 15.514 |
| 1817 | ASP222 | OD2 | 33.99 | 36.386 | 16.057 |
| 1818 | ASP222 | C | 33.884 | 39.222 | 13.262 |
| 1819 | ASP222 | O | 35.012 | 39.218 | 13.773 |
| 1820 | PRO223 | N | 33.077 | 40.274 | 13.286 |
| 1821 | PRO223 | CA | 33.573 | 41.635 | 13.541 |
| 1822 | PRO223 | CB | 32.432 | 42.527 | 13.165 |
| 1823 | PRO223 | CG | 31.195 | 41.686 | 12.891 |
| 1824 | PRO223 | CD | 31.64 | 40.24 | 12.999 |
| 1825 | PRO223 | C | 33.964 | 41.906 | 14.992 |
| 1826 | PRO223 | O | 34.672 | 42.875 | 15.279 |
| 1827 | ASN224 | N | 33.582 | 41.021 | 15.895 |
| 1828 | ASN224 | CA | 33.907 | 41.212 | 17.304 |
| 1829 | ASN224 | CB | 32.695 | 40.769 | 18.115 |
| 1830 | ASN224 | CG | 31.449 | 41.489 | 17.593 |
| 1831 | ASN224 | OD1 | 31.449 | 42.713 | 17.404 |
| 1832 | ASN224 | ND2 | 30.411 | 40.713 | 17.331 |
| 1833 | ASN224 | C | 35.155 | 40.422 | 17.697 |
| 1834 | ASN224 | O | 35.647 | 40.552 | 18.825 |
| 1835 | A3P225 | N | 35.7 | 39.664 | 16.757 |
| 1836 | ASP225 | CA | 36.896 | 38.87 | 17.038 |
| 1837 | ASP225 | CB | 36.889 | 37.64 | 16.134 |
| 1838 | ASP225 | CG | 37.893 | 36.588 | 16.6 |
| 1839 | ASP225 | OD1 | 39.022 | 36.962 | 16.894 |
| 1840 | ASP225 | OD2 | 37.568 | 35.416 | 16.489 |
| 1841 | ASP225 | C | 38.143 | 39.709 | 16.788 |
| 1842 | ASP225 | O | 38.667 | 39.745 | 15.666 |
| 1843 | GLN226 | N | 38.764 | 40.091 | 17.893 |
| 1844 | GLN226 | CA | 39.907 | 41.01 | 17.886 |
| 1845 | GLN226 | CB | 40.109 | 41.461 | 19.325 |
| 1846 | GLN226 | CG | 40.272 | 40.272 | 20.267 |
| 1847 | GLN226 | CD | 40.253 | 40.746 | 21.716 |
| 1848 | GLN226 | QE1 | 39.343 | 41.474 | 22.126 |
| 1849 | GLN226 | NE2 | 41.22 | 40.279 | 22.485 |
| 1850 | GLN226 | C | 41.225 | 40.452 | 17.34 |
| 1851 | GLN226 | O | 42.081 | 41.257 | 16.952 |
| 1852 | SER227 | N | 41.296 | 39.159 | 17.054 |
| 1853 | SER227 | CA | 42.549 | 38.59 | 16.555 |
| 1854 | SER227 | CB | 42.491 | 37.069 | 16.682 |
| 1855 | SER227 | OG | 41.519 | 36.528 | 15.791 |
| 1856 | SER227 | C | 42.808 | 38.988 | 15.103 |
| 1857 | SER227 | O | 43.943 | 39.351 | 14.773 |
| 1858 | ALA228 | N | 41.742 | 39.245 | 14.36 |
| 1859 | ALA228 | CA | 41.912 | 39.638 | 12.963 |
| 1860 | ALA228 | CB | 40.653 | 39.262 | 12.196 |
| 1861 | ALA228 | C | 42.182 | 41.134 | 12.836 |
| 1862 | ALA228 | O | 42.936 | 41.544 | 11.946 |
| 1863 | TRP229 | N | 41.835 | 41.875 | 13.877 |
| 1864 | TRP229 | CA | 42.075 | 43.318 | 13.887 |
| 1865 | TRP229 | CB | 41.114 | 43.966 | 14.876 |
| 1866 | TRP229 | CG | 39.655 | 43.71 | 14.574 |
| 1867 | TRP229 | OD1 | 38.819 | 42.825 | 15.218 |
| 1868 | TRP229 | NE1 | 37.588 | 42.903 | 14.652 |
| 1869 | TRP229 | CE2 | 37.572 | 43.805 | 13.656 |
| 1870 | TRP229 | CZ2 | 36.568 | 44.244 | 12.807 |
| 1871 | TRP229 | CH2 | 36.852 | 45.213 | 11.856 |
| 1872 | TRP229 | CZ3 | 38.131 | 45.753 | 11.756 |
| 1873 | TRP229 | CE3 | 39.139 | 45.325 | 12.609 |
| 1874 | TRP229 | CD2 | 38.861 | 44.354 | 13.557 |
| 1875 | TRP229 | C | 43.501 | 43.617 | 14.32 |
| 1876 | TRP229 | O | 44.179 | 44.442 | 13.692 |
| 1877 | PHE230 | N | 44.022 | 42.77 | 15.194 |
| 1878 | PHE230 | CA | 45.406 | 42.931 | 15.641 |
| 1879 | PHE230 | CB | 45.641 | 42.085 | 16.887 |
| 1880 | PHE230 | CG | 44.918 | 42.563 | 18.143 |
| 1881 | PHE230 | CO1 | 44.407 | 41.637 | 19.044 |
| 1882 | PHE230 | CE1 | 43.751 | 42.07 | 20.189 |
| 1883 | PHE230 | CZ | 43.611 | 43.429 | 20.438 |
| 1884 | PHE230 | CE2 | 44.13 | 44.355 | 19.542 |
| 1885 | PHE230 | CO2 | 44.785 | 43.923 | 18.397 |
| 1886 | PHE230 | C | 46.379 | 42.504 | 14.552 |
| 1887 | PHE230 | O | 47.341 | 43.234 | 14.277 |
| 1888 | TYR231 | N | 45.994 | 41.509 | 13.768 |
| 1889 | TYR231 | CA | 46.881 | 41.093 | 12.687 |
| 1890 | TYR231 | CB | 46.587 | 39.653 | 12.302 |
| 1891 | TYR231 | CG | 47.747 | 39.01 | 11.552 |
| 1892 | TYR231 | CD1 | 48.992 | 38.944 | 12.163 |
| 1893 | TYR231 | CE1 | 50.061 | 38.36 | 11.499 |
| 1894 | TYR231 | CZ | 49.883 | 37.844 | 10.224 |
| 1895 | TYR231 | OH | 50.938 | 37.234 | 9.584 |
| 1896 | TYR231 | CE2 | 48.643 | 37.915 | 9.605 |
| 1897 | TYR231 | CD2 | 47.574 | 38.502 | 10.271 |
| 1898 | TYR231 | C | 46.745 | 42.007 | 11.47 |
| 1899 | TYR231 | O | 47.764 | 42.285 | 10.829 |
| 1900 | HIS232 | N | 45.615 | 42.688 | 11.338 |
| 1901 | HIS232 | CA | 45.461 | 43.669 | 10.259 |
| 1902 | HIS232 | CB | 43.99 | 44.052 | 10.129 |
| 1903 | HIS232 | CG | 43.697 | 45.029 | 9.004 |
| 1904 | HIS232 | ND1 | 43.473 | 44.723 | 7.712 |
| 1905 | HIS232 | GE1 | 43.25 | 45.855 | 7.015 |
| 1906 | HIS232 | NE2 | 43.336 | 46.891 | 7.88 |
| 1907 | HIS232 | CD2 | 43.608 | 46.398 | 9.11 |
| 1908 | HIS232 | C | 46.28 | 44.922 | 10.544 |
| 1909 | HIS232 | O | 46.973 | 45.404 | 9.639 |
| 1910 | ARG233 | N | 46.433 | 45.256 | 11.816 |
| 1911 | ARG233 | CA | 47.267 | 46.405 | 12.178 |
| 1912 | ARG233 | CB | 46.906 | 46.85 | 13.593 |
| 1913 | ARG233 | OG | 47.64 | 48.133 | 13.972 |
| 1914 | ARG233 | CD | 47.261 | 48.62 | 15.366 |
| 1915 | ARG233 | NE | 47.944 | 49.888 | 15.673 |
| 1916 | ARG233 | CZ | 47.365 | 50.902 | 16.32 |
| 1917 | ARG233 | NH1 | 46.105 | 50.789 | 16.746 |
| 1918 | ARG233 | NH2 | 48.048 | 52.025 | 16.552 |
| 1919 | ARG233 | C | 48.757 | 46.062 | 12.096 |
| 1920 | ARG233 | O | 49.551 | 46.92 | 11.692 |
| 1921 | TRP234 | N | 49.083 | 44.782 | 12.196 |
| 1922 | TRP234 | CA | 50.475 | 44.357 | 12.02 |
| 1923 | TRP234 | CB | 50.641 | 42.951 | 12.592 |
| 1924 | TRP234 | CG | 52.071 | 42.442 | 12.578 |
| 1925 | TRP234 | CD1 | 53.023 | 42.667 | 13.548 |
| 1926 | TRP234 | NE1 | 54.175 | 42.056 | 13.172 |
| 1927 | TRP234 | CE2 | 54.031 | 41.43 | 11.99 |
| 1928 | TRP234 | CZ2 | 54.906 | 40.696 | 11.202 |
| 1929 | TRP234 | CH2 | 54.464 | 40.156 | 10 |
| 1930 | TRP234 | CZ3 | 53.152 | 40.351 | 9.583 |
| 1931 | TRP234 | CE3 | 52.271 | 41.09 | 10.365 |
| 1932 | TRP234 | CD2 | 52.706 | 41.632 | 11.563 |
| 1933 | TRP234 | C | 50.859 | 44.347 | 10.542 |
| 1934 | TRP234 | O | 51.943 | 44.83 | 10.197 |
| 1935 | LEU235 | N | 49.892 | 44.062 | 9.683 |
| 1936 | LEU235 | CA | 50.128 | 44.054 | 8.231 |
| 1937 | LEU235 | CB | 49.029 | 43.219 | 7.592 |
| 1938 | LEU235 | CG | 49.053 | 41.78 | 8.079 |
| 1939 | LEU235 | CD1 | 47.736 | 41.084 | 7.769 |
| 1940 | LEU235 | CD2 | 50.239 | 41.017 | 7.506 |
| 1941 | LEU235 | C | 50.068 | 45.456 | 7.628 |
| 1942 | LEU235 | O | 50.586 | 45.695 | 6.531 |
| 1943 | LEU236 | N | 49.48 | 46.377 | 8.372 |
| 1944 | LEU236 | CA | 49.418 | 47.78 | 7.966 |
| 1945 | LEU236 | OB | 48.109 | 48.342 | 8.515 |
| 1946 | LEU236 | CG | 47.73 | 49.673 | 7.878 |
| 1947 | LEU236 | OD1 | 47.582 | 49.517 | 6.369 |
| 1948 | LEU236 | CD2 | 46.442 | 50.214 | 8.487 |
| 1949 | LEU236 | C | 50.611 | 48.555 | 8.533 |
| 1950 | LEU236 | O | 50.86 | 49.705 | 8.148 |
| 1951 | GLY237 | N | 51.377 | 47.894 | 9.387 |
| 1952 | GLY237 | CA | 52.548 | 48.512 | 10.002 |
| 1953 | GLY237 | C | 53.713 | 48.628 | 9.028 |
| 1954 | GLY237 | O | 53.719 | 48.045 | 7.936 |
| 1955 | ARG238 | N | 54.645 | 49.479 | 9.413 |
| 1956 | ARG238 | CA | 55.831 | 49.742 | 8.605 |
| 1957 | ARG238 | CB | 56.201 | 51.2 | 8.804 |
| 1958 | ARG238 | CG | 55.042 | 52.123 | 8.46 |
| 1959 | ARG238 | CD | 55.354 | 53.55 | 8.891 |
| 1960 | ARG238 | NE | 55.551 | 53.625 | 10.349 |
| 1961 | ARG238 | CZ | 56.685 | 54.03 | 10.928 |
| 1962 | ARG238 | NH1 | 57.736 | 54.37 | 10.181 |
| 1963 | ARG238 | NH2 | 56.773 | 54.075 | 12.259 |
| 1964 | ARG238 | C | 57.012 | 48.885 | 9.041 |
| 1965 | ARG238 | O | 57.183 | 48.585 | 10.231 |
| 1966 | ALA239 | N | 57.828 | 48.513 | 8.072 |
| 1967 | ALA239 | CA | 59.082 | 47.814 | 8.364 |
| 1968 | ALA239 | CB | 59.543 | 47.064 | 7.121 |
| 1969 | ALA239 | C | 60.152 | 48.817 | 8.784 |
| 1970 | ALA239 | O | 60.785 | 49.474 | 7.948 |
| 1971 | ASP24O | N | 60.311 | 48.955 | 10.089 |
| 1972 | ASP24O | CA | 61.326 | 49.852 | 10.65 |
| 1973 | ASP24O | CB | 61.039 | 49.994 | 12.143 |
| 1974 | ASP24O | CG | 61.91 | 51.072 | 12.786 |
| 1975 | ASP24O | OD1 | 62.053 | 52.121 | 12.173 |
| 1976 | ASP24O | OD2 | 62.265 | 50.892 | 13.942 |
| 1977 | ASP24O | C | 62.72 | 49.272 | 10.421 |
| 1978 | ASP24O | O | 62.982 | 48.112 | 10.757 |
| 1979 | PRO241 | N | 63.578 | 50.06 | 9.791 |
| 1980 | PRO241 | CA | 64.949 | 49.634 | 9.481 |
| 1981 | PRO241 | CB | 65.488 | 50.691 | 8.564 |
| 1982 | PRO241 | CG | 64.49 | 51.832 | 8.469 |
| 1983 | PRO241 | CD | 63.287 | 51 .406 | 9.292 |
| 1984 | PRO241 | C | 65.824 | 49.515 | 10.73 |
| 1985 | PRO241 | O | 65.342 | 49.265 | 11.844 |
| 1986 | GLN242 | N | 67.125 | 49.509 | 10.497 |
| 1987 | GLN242 | CA | 68.084 | 49.557 | 11.604 |
| 1988 | GLN242 | CB | 68.549 | 48.129 | 11.896 |
| 1989 | GLN242 | CG | 69.303 | 47.973 | 13.222 |
| 1990 | GLN242 | CD | 68.403 | 48.002 | 14.469 |
| 1991 | GLN242 | OE1 | 68.922 | 47.941 | 15.59 |
| 1992 | GLN242 | NE2 | 67.092 | 48.044 | 14.287 |
| 1993 | GLN242 | C | 69.238 | 50.486 | 11.231 |
| 1994 | GLN242 | O | 70.248 | 50.627 | 11.932 |
| 1995 | ASP243 | N | 69 | 51.201 | 10.149 |
| 1996 | ASP243 | CA | 70.014 | 52.057 | 9.542 |
| 1997 | ASP243 | CB | 70.642 | 51.301 | 8.359 |
| 1998 | ASP243 | CG | 69.608 | 50.707 | 7.389 |
| 1999 | ASP243 | OD1 | 69.053 | 49.66 | 7.707 |
| 2000 | ASP243 | OD2 | 69.395 | 51.305 | 6.346 |
| 20D1 | ASP243 | C | 69.398 | 53.384 | 9.107 |
| 20D2 | ASP243 | O | 68.97 | 53.542 | 7.957 |
| 2003 | ALA244 | N | 69.354 | 54.331 | 10.028 |
| 2004 | ALA244 | CA | 68.753 | 55.627 | 9.701 |
| 2005 | ALA244 | CB | 67.237 | 55.496 | 9.777 |
| 2006 | ALA244 | C | 69.216 | 56.773 | 10.598 |
| 2007 | ALA244 | O | 68.821 | 56.88 | 11.768 |
| 2008 | LEU245 | N | 70.074 | 57.61 | 10.037 |
| 2009 | LEU245 | CA | 70.447 | 58.874 | 10.688 |
| 2010 | LEU245 | CB | 71.886 | 59.232 | 10.341 |
| 2011 | LEU245 | CG | 72.877 | 58.161 | 10.772 |
| 2012 | LEU245 | CD1 | 74.278 | 58.508 | 10.283 |
| 2013 | LEU245 | CD2 | 72.865 | 57.98 | 12.282 |
| 2014 | LEU245 | C | 69.524 | 59.942 | 10.132 |
| 2015 | LEU245 | O | 69.834 | 60.565 | 9.112 |
| 2016 | ARG246 | N | 68.46 | 60.23 | 10.857 |
| 2017 | ARG246 | CA | 67.362 | 60.966 | 10.239 |
| 2018 | ARG246 | CB | 66.064 | 60.592 | 10.94 |
| 2019 | ARG246 | CG | 65.84 | 59.084 | 10.872 |
| 2020 | ARG246 | CD | 64.398 | 58.74 | 11.217 |
| 2021 | ARG246 | NE | 64.16 | 57.288 | 11.279 |
| 2022 | ARG246 | CZ | 63.746 | 56.522 | 10.264 |
| 2023 | ARG246 | NH1 | 63.595 | 57.041 | 9.042 |
| 2024 | ARG246 | NH2 | 63.542 | 55.217 | 10.46 |
| 2025 | ARG246 | C | 67.53 | 62.479 | 10.221 |
| 2026 | ARG246 | O | 66.905 | 63.123 | 9.372 |
| 2027 | CYS247 | N | 68.428 | 63.035 | 11.015 |
| 2028 | CYS247 | CA | 68.612 | 64.49 | 10.941 |
| 2029 | CYS247 | CR | 67.529 | 65.167 | 11.774 |
| 2030 | CYS247 | 5G | 67.568 | 66.973 | 11.773 |
| 2031 | CYS247 | C | 69.98 | 64.963 | 11.417 |
| 2032 | CYS247 | O | 70.23 | 65.06 | 12.626 |
| 2033 | LEU248 | N | 70.838 | 65.291 | 10.466 |
| 2034 | LEU248 | CA | 72.111 | 65.945 | 10.799 |
| 2035 | LEU248 | CR | 73.143 | 65.761 | 9.694 |
| 2036 | LEU248 | CG | 73.587 | 64.325 | 9.478 |
| 2037 | LEU248 | CO1 | 74.794 | 64.332 | 8.548 |
| 2038 | LEU248 | CD2 | 73.96 | 63.659 | 10.795 |
| 2039 | LEU248 | C | 71.908 | 67.444 | 10.943 |
| 2040 | LEU248 | O | 71.003 | 68.019 | 10.322 |
| 2041 | HIS249 | N | 72.738 | 68.059 | 11.762 |
| 2042 | HIS249 | CA | 72.762 | 69.519 | 11.843 |
| 2043 | HIS249 | CR | 71.626 | 69.992 | 12.736 |
| 2044 | HIS249 | CG | 71.601 | 71.497 | 12.858 |
| 2045 | HIS249 | NO1 | 71.255 | 72.362 | 11.889 |
| 2046 | HIS249 | CE1 | 71.367 | 73.619 | 12.357 |
| 2047 | HIS249 | NE2 | 71.802 | 73.545 | 13.635 |
| 2048 | HIS249 | CD2 | 71.954 | 72.242 | 13.959 |
| 2049 | HIS249 | C | 74.075 | 70.056 | 12.405 |
| 2050 | HIS249 | O | 74.352 | 69.914 | 13.602 |
| 2051 | VAL250 | N | 74.86 | 70.695 | 11.556 |
| 2052 | VAL250 | CA | 76.046 | 71.392 | 12.057 |
| 2053 | VAL250 | CB | 77.219 | 71.283 | 11.084 |
| 2054 | VAL250 | CG1 | 77.82 | 69.889 | 11.094 |
| 2055 | VAL250 | CG2 | 76.869 | 71.712 | 9.665 |
| 2056 | VAL250 | C | 75.737 | 72.859 | 12.328 |
| 2057 | VAL250 | O | 75.3 | 73.615 | 11.45 |
| 2058 | SER251 | N | 75.893 | 73.233 | 13.579 |
| 2059 | SER251 | CA | 75.807 | 74.64 | 13.93 |
| 2060 | SER251 | CB | 75.082 | 74.8 | 15.256 |
| 2061 | SER251 | OG | 75.196 | 76.17 | 15.615 |
| 2062 | SER251 | C | 77.203 | 75.22 | 14.054 |
| 2063 | SER251 | O | 77.958 | 74.851 | 14.961 |
| 2064 | ARG252 | N | 77.463 | 76.245 | 13.263 |
| 2065 | ARG252 | CA | 78.733 | 76.962 | 13.347 |
| 2066 | ARG252 | CB | 78.946 | 77.742 | 12.053 |
| 2067 | ARG252 | CG | 80.243 | 78.544 | 12.083 |
| 2068 | ARG252 | CD | 80.45 | 79.341 | 10.798 |
| 2069 | ARG252 | NE | 80.612 | 78.455 | 9.634 |
| 2070 | ARG252 | CZ | 80.957 | 78.9 | 8.424 |
| 2071 | ARG252 | NH1 | 81.165 | 80.204 | 8.229 |
| 2072 | ARG252 | NH2 | 81.096 | 78.044 | 7.409 |
| 2073 | ARG252 | C | 78.678 | 77.919 | 14.53 |
| 2074 | ARG252 | O | 79.661 | 78.042 | 15.269 |
| 2075 | ASP253 | N | 77.46 | 78.314 | 14.873 |
| 2076 | ASP253 | CA | 77.229 | 79.174 | 16.042 |
| 2077 | ASP253 | CB | 75.749 | 79.533 | 16.11 |
| 2078 | ASP253 | CG | 75.244 | 80.072 | 14.78 |
| 2079 | ASP253 | OD1 | 75.759 | 81.09 | 14.334 |
| 2080 | ASP253 | OD2 | 74.352 | 79.447 | 14.223 |
| 2081 | ASP253 | C | 77.579 | 78.458 | 17.343 |
| 2082 | ASP253 | O | 78.358 | 78.977 | 18.148 |
| 2083 | GLU254 | N | 77.107 | 77.227 | 17.485 |
| 2084 | GLU254 | CA | 77.392 | 76.458 | 18.705 |
| 2085 | GLU254 | CB | 76.258 | 75.46 | 18.94 |
| 2086 | GLU254 | CG | 74.87 | 76.092 | 18.939 |
| 2087 | GLU254 | CD | 74.739 | 77.163 | 20.015 |
| 2088 | GLU254 | OE1 | 74.231 | 76.836 | 21.078 |
| 2089 | GLU254 | OE2 | 74.933 | 78.316 | 19.656 |
| 2090 | GLU254 | C | 78.69 | 75.653 | 18.632 |
| 2091 | GLU254 | O | 79.071 | 75.06 | 19.649 |
| 2092 | ALA255 | N | 79.38 | 75.703 | 17.5 |
| 2093 | ALA255 | CA | 80.48 | 74.774 | 17.202 |
| 2094 | ALA255 | CB | 81.725 | 75.192 | 17.978 |
| 2095 | ALA255 | C | 80.078 | 73.348 | 17.566 |
| 2096 | ALA255 | O | 80.707 | 72.716 | 18.427 |
| 2097 | CYS256 | N | 79.048 | 72.842 | 16.905 |
| 2098 | CYS256 | CA | 78.488 | 71.546 | 17.312 |
| 2099 | CYS256 | CB | 77.596 | 71.801 | 18.524 |
| 2100 | GY5256 | SG | 76.875 | 70.343 | 19.312 |
| 2101 | GY5256 | C | 77.675 | 70.849 | 16.22 |
| 2102 | GY5256 | O | 76.751 | 71.424 | 15.631 |
| 2103 | LEU257 | N | 78.014 | 69.591 | 15.994 |
| 2104 | LEU257 | CA | 77.259 | 68.727 | 15.075 |
| 2105 | LEU257 | CB | 78.249 | 67.886 | 14.271 |
| 2106 | LEU257 | CG | 77.613 | 66.691 | 13.551 |
| 2107 | LEU257 | CD1 | 76.533 | 67.087 | 12.548 |
| 2108 | LEU257 | CD2 | 78.685 | 65.868 | 12.857 |
| 2109 | LEU257 | C | 76.311 | 67.821 | 15.859 |
| 2110 | LEU257 | O | 76.743 | 66.985 | 16.661 |
| 2111 | THR258 | N | 75.025 | 68.01 | 15.625 |
| 2112 | THR258 | CA | 73.992 | 67.195 | 16.266 |
| 2113 | THR258 | CB | 72.887 | 68.15 | 16.701 |
| 2114 | THR258 | OG1 | 73.503 | 69.235 | 17.382 |
| 2115 | THR258 | CG2 | 71.885 | 67.492 | 17.642 |
| 2116 | THR258 | C | 73.438 | 66.148 | 15.296 |
| 2117 | THR258 | O | 73.237 | 66.436 | 14.111 |
| 2118 | VAL259 | N | 73.334 | 64.916 | 15.767 |
| 2119 | VAL259 | CA | 72.716 | 63.842 | 14.978 |
| 2120 | VAL259 | CB | 73.729 | 62.711 | 14.815 |
| 2121 | VAL259 | CG1 | 73.15 | 61.553 | 14.008 |
| 2122 | VAL259 | CG2 | 75.01 | 63.216 | 14.165 |
| 2123 | VAL259 | C | 71.456 | 63.294 | 15.655 |
| 2124 | VAL259 | O | 71.509 | 62.756 | 16.771 |
| 2125 | SER260 | N | 70.328 | 63.495 | 14.995 |
| 2126 | SER260 | CA | 69.067 | 62.891 | 15.433 |
| 2127 | SER260 | CB | 67.901 | 63.797 | 15.068 |
| 2128 | SER260 | OG | 68.052 | 65.009 | 15.792 |
| 2129 | SER260 | C | 68.877 | 61.516 | 14.8 |
| 2130 | SER260 | O | 68.975 | 61.329 | 13.578 |
| 2131 | PHE261 | N | 68.63 | 60.561 | 15.673 |
| 2132 | PHE261 | CA | 68.479 | 59.158 | 15.294 |
| 2133 | PHE261 | CB | 69.106 | 58.285 | 16.376 |
| 2134 | PHE261 | CG | 70.629 | 58.247 | 16.383 |
| 2135 | PHE261 | CD1 | 71.359 | 59.184 | 17.102 |
| 2136 | PHE261 | CE1 | 72.746 | 59.131 | 17.098 |
| 2137 | PHE261 | CZ | 73.401 | 58.138 | 16.383 |
| 2138 | PHE261 | CE2 | 72.672 | 57.199 | 15.669 |
| 2139 | PHE261 | CD2 | 71.285 | 57.256 | 15.668 |
| 2140 | PHE261 | C | 67.025 | 58.749 | 15.148 |
| 2141 | PHE261 | O | 66.088 | 59.556 | 15.208 |
| 2142 | SER262 | N | 66.872 | 57.467 | 14.883 |
| 2143 | SER262 | CA | 65.551 | 56.852 | 14.838 |
| 2144 | SER262 | CB | 65.662 | 55.551 | 14.057 |
| 2145 | SER262 | OG | 66.344 | 55.819 | 12.841 |
| 2146 | SER262 | C | 65.142 | 56.523 | 16.263 |
| 2147 | SER262 | O | 64.689 | 57.384 | 17.029 |
| 2148 | ARG263 | N | 65.399 | 55.274 | 16.61 |
| 2149 | ARG263 | CA | 65.213 | 54.751 | 17.966 |
| 2150 | ARG263 | CB | 65.281 | 53.231 | 17.834 |
| 2151 | ARG263 | CG | 66.659 | 52.799 | 17.349 |
| 2152 | ARG263 | CD | 66.622 | 51.472 | 16.597 |
| 2153 | ARG263 | NE | 65.873 | 51.613 | 15.335 |
| 2154 | ARG263 | CZ | 66.434 | 51.961 | 14.173 |
| 2155 | ARG263 | NH1 | 65.669 | 52.158 | 13.097 |
| 2156 | ARG263 | NH2 | 67.749 | 52.189 | 14.102 |
| 2157 | ARG263 | C | 66.323 | 55.284 | 18.88 |
| 2158 | ARG263 | O | 67.296 | 55.858 | 18.374 |
| 2159 | PRO264 | N | 66.121 | 55.222 | 20.19 |
| 2160 | PRO264 | CA | 67.153 | 55.67 | 21.132 |
| 2161 | PRO264 | CB | 66.502 | 55.637 | 22.479 |
| 2162 | PRO264 | CG | 65.129 | 54.996 | 22.355 |
| 2163 | PRO264 | CD | 64.929 | 54.711 | 20.876 |
| 2164 | PRO264 | G | 68.37 | 54.753 | 21.089 |
| 2165 | PRO264 | O | 68.331 | 53.608 | 21.553 |
| 2166 | LEU265 | N | 69.455 | 55.284 | 20.559 |
| 2167 | LEU265 | CA | 70.68 | 54.501 | 20.401 |
| 2168 | LEU265 | CB | 71.122 | 54.572 | 18.944 |
| 2169 | LEU265 | CG | 70.174 | 53.763 | 18.065 |
| 2170 | LEU265 | CD1 | 70.431 | 53.992 | 16.581 |
| 2171 | LEU265 | CD2 | 70.256 | 52.278 | 18.404 |
| 2172 | LEU265 | C | 71.793 | 54.969 | 21.327 |
| 2173 | LEU265 | O | 71.618 | 55.877 | 22.15 |
| 2174 | LEU266 | N | 72.871 | 54.209 | 21.294 |
| 2175 | LEU266 | CA | 74.073 | 54.517 | 22.074 |
| 2176 | LEU266 | CB | 74.288 | 53.411 | 23.1 |
| 2177 | LEU266 | CG | 73.487 | 53.636 | 24.372 |
| 2178 | LEU266 | CD1 | 73.473 | 52.383 | 25.239 |
| 2179 | LEU266 | CD2 | 74.06 | 54.818 | 25.141 |
| 2180 | LEU266 | C | 75.303 | 54.588 | 21.181 |
| 2181 | LEU266 | O | 75.776 | 53.556 | 20.691 |
| 2182 | VAL267 | N | 75.832 | 55.784 | 20.996 |
| 2183 | VAL267 | CA | 77.076 | 55.924 | 20.233 |
| 2184 | VAL267 | CB | 77.193 | 57.348 | 19.706 |
| 2185 | VAL267 | CG1 | 78.505 | 57.552 | 18.961 |
| 2186 | VAL267 | CG2 | 76.017 | 57.669 | 18.797 |
| 2187 | VAL267 | C | 78.262 | 55.569 | 21.124 |
| 2188 | VAL267 | O | 78.675 | 56.337 | 22.001 |
| 2189 | GLY268 | N | 78.771 | 54.374 | 20.893 |
| 2190 | GLY268 | CA | 79.857 | 53.813 | 21.69 |
| 2191 | GLY268 | C | 79.424 | 52.467 | 22.258 |
| 2192 | GLY268 | O | 80.055 | 51.944 | 23.185 |
| 2193 | SER269 | N | 78.349 | 51.921 | 21.713 |
| 2194 | SER269 | CA | 77.838 | 50.639 | 22.216 |
| 2195 | SER269 | CB | 76.318 | 50.592 | 22.095 |
| 2196 | SER269 | OG | 75.952 | 50.738 | 20.73 |
| 2197 | SER269 | C | 78.459 | 49.448 | 21.493 |
| 2198 | SER269 | O | 79.583 | 49.522 | 20.978 |
| 2199 | ARG270 | N | 77.746 | 48.334 | 21.568 |
| 2200 | ARG270 | CA | 78.146 | 47.075 | 20.922 |
| 2201 | ARG270 | CB | 76.969 | 46.117 | 21.051 |
| 2202 | ARG270 | CG | 76.525 | 46.016 | 22.505 |
| 2203 | ARG270 | CD | 75.191 | 45.294 | 22.634 |
| 2204 | ARG270 | NE | 75.271 | 43.924 | 22.109 |
| 2205 | ARG270 | CZ | 74.368 | 42.988 | 22.405 |
| 2206 | ARG270 | NH1 | 73.33 | 43.287 | 23.189 |
| 2207 | ARG270 | NH2 | 74.494 | 41.757 | 21.905 |
| 2208 | ARG270 | C | 78.444 | 47.339 | 19.454 |
| 2209 | ARG270 | O | 79.601 | 47.279 | 19.018 |
| 2210 | MET271 | N | 77.404 | 47.668 | 18.709 |
| 2211 | MET271 | CA | 77.628 | 48.268 | 17.399 |
| 2212 | MET271 | CB | 76.418 | 48.048 | 16.514 |
| 2213 | MET271 | CG | 76.871 | 47.441 | 15.193 |
| 2214 | MET271 | SD | 77.802 | 45.897 | 15.313 |
| 2215 | MET271 | CE | 78.163 | 45.671 | 13.558 |
| 2216 | MET271 | C | 77.905 | 49.738 | 17.681 |
| 2217 | MET271 | O | 77.05 | 50.461 | 18.204 |
| 2218 | GLU272 | N | 79.098 | 50.166 | 17.325 |
| 2219 | GLU272 | CA | 79.709 | 51.303 | 18.015 |
| 2220 | GLU272 | CB | 81.211 | 51.206 | 17.803 |
| 2221 | GLU272 | CG | 81.745 | 49.951 | 18.486 |
| 2222 | GLU272 | CD | 83.235 | 49.796 | 18.214 |
| 2223 | GLU272 | OE1 | 84.012 | 50.428 | 18.916 |
| 2224 | GLU272 | OE2 | 83.551 | 49.206 | 17.189 |
| 2225 | GLU272 | C | 79.214 | 52.716 | 17.704 |
| 2226 | GLU272 | O | 78.275 | 53.193 | 18.352 |
| 2227 | ILEA273 | N | 79.793 | 53.344 | 16.697 |
| 2228 | ILEA273 | CA | 79.841 | 54.816 | 16.691 |
| 2229 | ILEA273 | CB | 81.266 | 55.255 | 17.032 |
| 2230 | ILEA273 | CG2 | 81.596 | 55.043 | 18.504 |
| 2231 | ILEA273 | CG1 | 82.283 | 54.546 | 16.143 |
| 2232 | ILEA273 | CD1 | 83.706 | 54.986 | 16.468 |
| 2233 | ILEA273 | C | 79.476 | 55.466 | 15.362 |
| 2234 | ILEA273 | O | 78.996 | 54.819 | 14.423 |
| 2235 | LEU274 | N | 79.593 | 56.786 | 15.371 |
| 2236 | LEU274 | CA | 79.457 | 57.608 | 14.164 |
| 2237 | LEU274 | CB | 78.585 | 58.814 | 14.488 |
| 2238 | LEU274 | CG | 77.168 | 58.442 | 14.898 |
| 2239 | LEU274 | OD1 | 76.456 | 59.647 | 15.498 |
| 2240 | LEU274 | OD2 | 76.391 | 57.891 | 13.711 |
| 2241 | LEU274 | C | 80.821 | 58.138 | 13.722 |
| 2242 | LEu274 | O | 81.483 | 58.875 | 14.465 |
| 2243 | LEU275 | N | 81.214 | 57.793 | 12.511 |
| 2244 | LEU275 | CA | 82.468 | 58.308 | 11.946 |
| 2245 | LEU275 | CB | 82.974 | 57.331 | 10.892 |
| 2246 | LEU275 | OG | 83.284 | 55.962 | 11.482 |
| 2247 | LEU275 | OD1 | 83.634 | 54.967 | 10.38 |
| 2248 | LEU275 | OD2 | 84.406 | 56.045 | 12.512 |
| 2249 | LEU275 | C | 82.248 | 59.666 | 11.29 |
| 2250 | LEU275 | O | 81.483 | 59.777 | 10.323 |
| 2251 | LEU276 | N | 82.896 | 60.685 | 11.824 |
| 2252 | LEU276 | CA | 82.789 | 62.02 | 11.231 |
| 2253 | LEU276 | OB | 82.933 | 63.068 | 12.331 |
| 2254 | LEU276 | CG | 82.772 | 64.494 | 11.805 |
| 2255 | LEU276 | OD1 | 81.464 | 64.671 | 11.042 |
| 2256 | LEU276 | OD2 | 82.864 | 65.51 | 12.934 |
| 2257 | LEU276 | C | 83.846 | 62.221 | 10.147 |
| 2258 | LEU276 | O | 85.047 | 62.019 | 10.362 |
| 2259 | MET277 | N | 83.365 | 62.531 | 8.958 |
| 2260 | MET277 | CA | 84.233 | 62.836 | 7.823 |
| 2261 | MET277 | OB | 83.872 | 61.907 | 6.671 |
| 2262 | MET277 | OG | 84.065 | 60.444 | 7.048 |
| 2263 | MET277 | SD | 85.759 | 59.958 | 7.445 |
| 2264 | MET277 | CE | 86.561 | 60.426 | 5.894 |
| 2265 | MET277 | C | 84.057 | 64.287 | 7.385 |
| 2266 | MET277 | O | 83.119 | 64.63 | 6.652 |
| 2267 | VAL278 | N | 84.986 | 65.118 | 7.821 |
| 2268 | VAL278 | CA | 84.992 | 66.531 | 7.44 |
| 2269 | VAL278 | CB | 85.671 | 67.349 | 8.532 |
| 2270 | VAL278 | OG1 | 85.705 | 68.831 | 8.17 |
| 2271 | VAL278 | CG2 | 84.967 | 67.144 | 9.865 |
| 2272 | VAL278 | C | 85.745 | 66.681 | 6.126 |
| 2273 | VAL278 | O | 86.983 | 66.76 | 6.096 |
| 2274 | ASP279 | N | 84.966 | 66.841 | 5.067 |
| 2275 | ASP279 | CA | 85.418 | 66.838 | 3.66 |
| 2276 | ASP279 | CB | 86.325 | 68.045 | 3.421 |
| 2277 | ASP279 | CG | 85.555 | 69.337 | 3.689 |
| 2278 | ASP279 | OD1 | 84.686 | 69.646 | 2.888 |
| 2279 | ASP279 | OD2 | 85.732 | 69.902 | 4.761 |
| 2280 | ASP279 | C | 86.114 | 65.533 | 3.248 |
| 2281 | ASP279 | O | 85.553 | 64.745 | 2.48 |
| 2282 | ASP280 | N | 87.344 | 65.341 | 3.695 |
| 2283 | ASP280 | CA | 88.073 | 64.1 | 3.426 |
| 2284 | ASP280 | CB | 89.095 | 64.333 | 2.312 |
| 2285 | ASP280 | CG | 90.094 | 65.433 | 2.678 |
| 2286 | ASP280 | OD1 | 91.145 | 65.101 | 3.206 |
| 2287 | ASP280 | OD2 | 89.794 | 66.586 | 2.392 |
| 2288 | ASP280 | C | 88.763 | 63.594 | 4.694 |
| 2289 | ASP280 | O | 89.252 | 62.46 | 4.735 |
| 2290 | SER281 | N | 88.755 | 64.417 | 5.73 |
| 2291 | SER281 | CA | 89.447 | 64.072 | 6.976 |
| 2292 | SER281 | CB | 89.944 | 65.361 | 7.62 |
| 2293 | SER281 | OG | 90.424 | 65.028 | 8.916 |
| 2294 | SER281 | C | 88.543 | 63.356 | 7.968 |
| 2295 | SER281 | O | 87.474 | 63.865 | 8.324 |
| 2296 | PRO282 | N | 88.987 | 62.199 | 8.426 |
| 2297 | PRO282 | CA | 88.39 | 61.591 | 9.612 |
| 2298 | PRO282 | CB | 89.085 | 60.275 | 9.769 |
| 2299 | PRO282 | CG | 90.232 | 60.197 | 8.77 |
| 2300 | PRO282 | CD | 90.185 | 61.492 | 7.974 |
| 2301 | PRO282 | C | 88.608 | 62.486 | 10.826 |
| 2302 | PRO282 | O | 89.73 | 62.922 | 11.108 |
| 2303 | LEU283 | N | 87.517 | 62.816 | 11.49 |
| 2304 | LEU283 | CA | 87.592 | 63.658 | 12.682 |
| 2305 | LEU283 | CB | 86.774 | 64.922 | 12.441 |
| 2306 | LEU283 | CG | 87.028 | 65.97 | 13.521 |
| 2307 | LEU283 | CD1 | 88.51 | 66.32 | 13.601 |
| 2308 | LEU283 | CD2 | 86.201 | 67.226 | 13.276 |
| 2309 | LEU283 | C | 87.076 | 62.903 | 13.904 |
| 2310 | LEU283 | O | 85.901 | 62.517 | 13.984 |
| 2311 | ILEA284 | N | 87.973 | 62.71 | 14.857 |
| 2312 | ILEA284 | CA | 87.634 | 61.998 | 16.097 |
| 2313 | ILEA284 | CB | 88.909 | 61.386 | 16.676 |
| 2314 | ILEA284 | CG2 | 88.602 | 60.61 | 17.953 |
| 2315 | ILEA284 | CG1 | 89.585 | 60.468 | 15.661 |
| 2316 | ILEA284 | CD1 | 88.72 | 59.253 | 15.334 |
| 2317 | ILEA284 | C | 86.993 | 62.948 | 17.11 |
| 2318 | ILEA284 | O | 87.676 | 63.646 | 17.868 |
| 2319 | VAL285 | N | 85.676 | 63.022 | 17.041 |
| 2320 | VAL285 | CA | 84.904 | 63.88 | 17.942 |
| 2321 | VAL285 | CB | 83.859 | 64.6 | 17.108 |
| 2322 | VAL285 | CG1 | 84.475 | 65.756 | 16.333 |
| 2323 | VAL285 | CG2 | 83.153 | 63.622 | 16.177 |
| 2324 | VAL285 | C | 84.232 | 63.096 | 19.064 |
| 2325 | VAL285 | O | 83.856 | 61.928 | 18.909 |
| 2326 | GLU286 | N | 84.108 | 63.751 | 20.205 |
| 2327 | GLU286 | CA | 83.45 | 63.126 | 21.358 |
| 2328 | GLU286 | CB | 84.006 | 63.74 | 22.637 |
| 2329 | GLU286 | CG | 83.389 | 63.107 | 23.881 |
| 2330 | GLU286 | CD | 84.006 | 63.726 | 25.13 |
| 2331 | GLU286 | OE1 | 85.143 | 64.168 | 25.033 |
| 2332 | GLU286 | OE2 | 83.336 | 63.747 | 26.152 |
| 2333 | GLU286 | C | 81.938 | 63.324 | 21.306 |
| 2334 | GLU286 | O | 81.44 | 64.442 | 21.483 |
| 2335 | TRP287 | N | 81.24 | 62.236 | 21.029 |
| 2336 | TRP287 | CA | 79.774 | 62.238 | 21.005 |
| 2337 | TRP287 | CB | 79.294 | 61.061 | 20.163 |
| 2338 | TRP287 | CG | 79.727 | 61.099 | 18.712 |
| 2339 | TRP287 | CD1 | 80.763 | 60.396 | 18.134 |
| 2340 | TRP287 | NE1 | 80.811 | 60.711 | 16.813 |
| 2341 | TRP287 | CE2 | 79.848 | 61.593 | 16.489 |
| 2342 | TRP287 | CZ2 | 79.505 | 62.214 | 15.299 |
| 2343 | TRP287 | CH2 | 78.429 | 63.094 | 15.266 |
| 2344 | TRP287 | CZ3 | 77.699 | 63.357 | 16.421 |
| 2345 | TRP287 | CE3 | 78.04 | 62.743 | 17.62 |
| 2346 | TRP287 | CD2 | 79.114 | 61.869 | 17.657 |
| 2347 | TRP287 | C | 79.177 | 62.105 | 22.404 |
| 2348 | TRP287 | O | 79.64 | 61.312 | 23.237 |
| 2349 | ARG288 | N | 78.163 | 62.913 | 22.651 |
| 2350 | ARG288 | CA | 77.409 | 62.823 | 23.9 |
| 2351 | ARG288 | CB | 78.091 | 63.697 | 24.944 |
| 2352 | ARG288 | CG | 78.003 | 65.162 | 24.55 |
| 2353 | ARG288 | CD | 78.842 | 66.052 | 25.455 |
| 2354 | ARG288 | NE | 78.645 | 67.466 | 25.1 |
| 2355 | ARG288 | CZ | 79.319 | 68.105 | 24.14 |
| 2356 | ARG288 | NH1 | 80.286 | 67.482 | 23.46 |
| 2357 | ARG288 | NH2 | 79.042 | 69.384 | 23.882 |
| 2358 | ARG288 | C | 75.959 | 63.271 | 23.712 |
| 2359 | ARG288 | O | 75.628 | 64.067 | 22.825 |
| 2360 | THR289 | N | 75.085 | 62.681 | 24.503 |
| 2361 | THR289 | CA | 73.684 | 63.108 | 24.531 |
| 2362 | THR289 | CB | 72.874 | 61.951 | 25.118 |
| 2363 | THR289 | OG1 | 71.533 | 62.353 | 25.348 |
| 2364 | THR289 | CG2 | 73.441 | 61.506 | 26.448 |
| 2365 | THR289 | C | 73.604 | 64.386 | 25.37 |
| 2366 | THR289 | O | 74.442 | 64.57 | 26.262 |
| 2367 | PRO290 | N | 72.637 | 65.263 | 25.112 |
| 2368 | PRO290 | CA | 72.676 | 66.641 | 25.651 |
| 2369 | PRO290 | CB | 71 .577 | 67.375 | 24.946 |
| 2370 | PRO290 | CG | 70.809 | 66.41 | 24.061 |
| 2371 | PRO290 | CD | 71.552 | 65.09 | 24.138 |
| 2372 | PRO290 | C | 72.481 | 66.777 | 27.169 |
| 2373 | PRO290 | O | 72.536 | 67.892 | 27.695 |
| 2374 | ASP291 | N | 72.238 | 65.679 | 27.865 |
| 2375 | ASP291 | CA | 72.142 | 65.708 | 29.323 |
| 2376 | ASP291 | CB | 71.039 | 64.747 | 29.765 |
| 2377 | ASP291 | CG | 71.378 | 63.309 | 29.379 |
| 2378 | ASP291 | OD1 | 72.021 | 62.66 | 30.188 |
| 2379 | ASP291 | OD2 | 71.028 | 62.914 | 28.274 |
| 2380 | ASP291 | C | 73.47 | 65.342 | 29.996 |
| 2381 | ASP291 | O | 73.531 | 65.284 | 31.23 |
| 2382 | GLY292 | N | 74.489 | 65.016 | 29.212 |
| 2383 | GLY292 | CA | 75.804 | 64.687 | 29.781 |
| 2384 | GLY292 | C | 76.004 | 63.179 | 29.936 |
| 2385 | GLY292 | O | 76.975 | 62.609 | 29.422 |
| 2386 | ARG293 | N | 75.155 | 62.581 | 30.754 |
| 2387 | ARG293 | CA | 75.162 | 61.129 | 30.957 |
| 2388 | ARG293 | CB | 74.095 | 60.812 | 31.993 |
| 2389 | ARG293 | CG | 74.328 | 61.556 | 33.3 |
| 2390 | ARG293 | CD | 73.082 | 61.481 | 34.171 |
| 2391 | ARG293 | NE | 72.602 | 60.094 | 34.259 |
| 2392 | ARG293 | CZ | 71.454 | 59.756 | 34.849 |
| 2393 | ARG293 | NH1 | 70.698 | 60.694 | 35.424 |
| 2394 | ARG293 | NH2 | 71.069 | 58.479 | 34.875 |
| 2395 | ARG293 | C | 74.782 | 60.419 | 29.667 |
| 2396 | ARG293 | O | 73.629 | 60.509 | 29.238 |
| 2397 | ASN294 | N | 75.697 | 59.623 | 29.137 |
| 2398 | ASN294 | CA | 75.471 | 58.925 | 27.859 |
| 2399 | ASN294 | CB | 76.823 | 58.646 | 27.211 |
| 2400 | ASN294 | CG | 77.337 | 59.92 | 26.541 |
| 2401 | ASN294 | OD1 | 76.558 | 60.842 | 26.27 |
| 2402 | ASN294 | ND2 | 78.608 | 59.907 | 26.176 |
| 2403 | ASN294 | C | 74.645 | 57.638 | 27.97 |
| 2404 | ASN294 | O | 75.152 | 56.522 | 27.81 |
| 2405 | ARG295 | N | 73.36 | 57.832 | 28.215 |
| 2406 | ARG295 | CA | 72.36 | 56.761 | 28.228 |
| 2407 | ARG295 | CB | 71.46 | 57.001 | 29.44 |
| 2408 | ARG295 | CG | 71.077 | 58.468 | 29.59 |
| 2409 | ARG295 | CD | 70.343 | 58.698 | 30.905 |
| 2410 | ARG295 | NE | 70.17 | 60.133 | 31.174 |
| 2411 | ARG295 | CZ | 69.229 | 60.618 | 31.986 |
| 2412 | ARG295 | NH1 | 68.371 | 59.788 | 32.583 |
| 2413 | ARG295 | NH2 | 69.144 | 61.933 | 32.198 |
| 2414 | ARG295 | C | 71.601 | 56.795 | 26.9 |
| 2415 | ARG295 | O | 71.81 | 57.745 | 26.139 |
| 2416 | PRO296 | N | 70.869 | 55.736 | 26.565 |
| 2417 | PRO296 | CA | 70.252 | 55.621 | 25.233 |
| 2418 | PRO296 | CB | 69.44 | 54.364 | 25.268 |
| 2419 | PRO296 | CG | 69.705 | 53.64 | 26.578 |
| 2420 | PRO296 | CD | 70.673 | 54.516 | 27.358 |
| 2421 | PRO296 | C | 69.41 | 56.842 | 24.882 |
| 2422 | PRO296 | O | 68.479 | 57.228 | 25.598 |
| 2423 | SER297 | N | 69.777 | 57.445 | 23.768 |
| 2424 | SER297 | CA | 69.204 | 58.731 | 23.378 |
| 2425 | SER297 | CB | 70.203 | 59.808 | 23.794 |
| 2426 | SER297 | OG | 69.762 | 61.074 | 23.317 |
| 2427 | SER297 | C | 68.945 | 58.822 | 21.882 |
| 2428 | SER297 | O | 69.599 | 58.168 | 21.061 |
| 2429 | HIS298 | N | 67.961 | 59.634 | 21.542 |
| 2430 | HIS298 | CA | 67.679 | 59.937 | 20.145 |
| 2431 | HIS298 | CB | 66.243 | 60.424 | 20.032 |
| 2432 | HIS298 | CG | 65.151 | 59.469 | 20.463 |
| 2433 | HIS298 | NO1 | 64.566 | 58.527 | 19.702 |
| 2434 | HIS298 | CE1 | 63.621 | 57.893 | 20.424 |
| 2435 | HIS298 | NE2 | 63.611 | 58.443 | 21 .659 |
| 2436 | HIS298 | CD2 | 64.545 | 59.42 | 21 .697 |
| 2437 | HIS298 | C | 68.559 | 61.066 | 19.608 |
| 2438 | HIS298 | O | 68.541 | 61.308 | 18.397 |
| 2439 | VAL299 | N | 69.31 | 61.751 | 20.457 |
| 2440 | VAL299 | CA | 70.083 | 62.894 | 19.979 |
| 2441 | VAL299 | CB | 69.338 | 64.168 | 20.381 |
| 2442 | VAL299 | CG1 | 68.827 | 64.108 | 21.817 |
| 2443 | VAL299 | CG2 | 70.159 | 65.427 | 20.133 |
| 2444 | VAL299 | C | 71.503 | 62.852 | 20.537 |
| 2445 | VAL299 | O | 71.717 | 62.825 | 21.757 |
| 2446 | TRP300 | N | 72.448 | 62.713 | 19.622 |
| 2447 | TRP300 | CA | 73.868 | 62.663 | 19.983 |
| 2448 | TRP300 | CB | 74.427 | 61.292 | 19.623 |
| 2449 | TRP300 | CG | 73.938 | 60.18 | 20.529 |
| 2450 | TRP300 | CO1 | 72.742 | 59.5 | 20.45 |
| 2451 | TRP300 | NE1 | 72.694 | 58.598 | 21 .461 |
| 2452 | TRP300 | CE2 | 73.812 | 58.643 | 22.207 |
| 2453 | TRP300 | CZ2 | 74.212 | 57.959 | 23.344 |
| 2454 | TRP300 | CH2 | 75.459 | 58.216 | 23.898 |
| 2455 | TRP300 | CZ3 | 76.302 | 59.164 | 23.326 |
| 2456 | TRP300 | CE3 | 75.898 | 59.871 | 22.201 |
| 2457 | TRP300 | CD2 | 74.655 | 59.618 | 21.647 |
| 2458 | TRP300 | C | 74.649 | 63.753 | 19.265 |
| 2459 | TRP300 | O | 74.679 | 63.819 | 18.031 |
| 2460 | LEU301 | N | 75.269 | 64.614 | 20.047 |
| 2461 | LEU301 | CA | 76.007 | 65.742 | 19.48 |
| 2462 | LEU301 | CB | 75.338 | 67.094 | 19.801 |
| 2463 | LEU301 | CG | 75.01 | 67.483 | 21.256 |
| 2464 | LEU301 | CD1 | 73.752 | 66.832 | 21.819 |
| 2465 | LEU301 | CD2 | 76.17 | 67.425 | 22.241 |
| 2466 | LEU301 | C | 77.483 | 65.716 | 19.863 |
| 2467 | LEU301 | O | 77.886 | 65.074 | 20.838 |
| 2468 | CYS302 | N | 78.288 | 66.298 | 18.997 |
| 2469 | CYS302 | CA | 79.722 | 66.416 | 19.259 |
| 2470 | CYS302 | CB | 80.471 | 65.48 | 18.322 |
| 2471 | CYS302 | SG | 80.335 | 65.886 | 16.567 |
| 2472 | CYS302 | C | 80.204 | 67.839 | 19.016 |
| 2473 | CYS302 | O | 79.676 | 68.553 | 18.153 |
| 2474 | ASP303 | N | 81.211 | 68.241 | 19.771 |
| 2475 | ASP303 | CA | 81.831 | 69.547 | 19.523 |
| 2476 | ASP303 | CB | 82.799 | 69.912 | 20.64 |
| 2477 | ASP303 | CG | 82.027 | 70.362 | 21.874 |
| 2478 | ASP303 | OD1 | 80.913 | 70.836 | 21.707 |
| 2479 | ASP303 | OD2 | 82.546 | 70.173 | 22.966 |
| 2480 | ASP303 | C | 82.56 | 69.543 | 18.188 |
| 2481 | ASP303 | O | 83.279 | 68.6 | 17.839 |
| 2482 | LEU304 | N | 82.315 | 70.596 | 17.435 |
| 2483 | LEU304 | CA | 82.884 | 70.743 | 16.099 |
| 2484 | LEU304 | CB | 81.737 | 71.093 | 15.16 |
| 2485 | LEU304 | CG | 82.093 | 70.894 | 13.696 |
| 2486 | LEU304 | CO1 | 82.455 | 69.436 | 13.433 |
| 2487 | LEU304 | CD2 | 80.922 | 71.316 | 12.819 |
| 2488 | LEU304 | C | 83.927 | 71.857 | 16.11 |
| 2489 | LEU304 | O | 83.593 | 73.038 | 16.249 |
| 2490 | PRO305 | N | 85.18 | 71.465 | 15.962 |
| 2491 | PRO305 | CA | 86.304 | 72.39 | 16.138 |
| 2492 | PRO305 | CB | 87.534 | 71.54 | 16.057 |
| 2493 | PRO305 | CG | 87.136 | 70.102 | 15.763 |
| 2494 | PRO305 | CD | 85.617 | 70.088 | 15.722 |
| 2495 | PRO305 | C | 86.339 | 73.486 | 15.081 |
| 2496 | PRO305 | O | 85.788 | 73.339 | 13.983 |
| 2497 | ALA306 | N | 87.175 | 74.481 | 15.342 |
| 2498 | ALA306 | CA | 87.363 | 75.608 | 14.414 |
| 2499 | ALA306 | CB | 88.061 | 76.74 | 15.157 |
| 2500 | ALA306 | C | 88.173 | 75.239 | 13.167 |
| 2501 | ALA306 | O | 88.073 | 75.919 | 12.14 |
| 2502 | ALA307 | N | 88.752 | 74.048 | 13.168 |
| 2503 | ALA307 | CA | 89.4 | 73.515 | 11.967 |
| 2504 | ALA307 | CB | 90.358 | 72.404 | 12.383 |
| 2505 | ALA307 | C | 88.377 | 72.966 | 10.965 |
| 2506 | ALA307 | O | 88.714 | 72.738 | 9.799 |
| 2507 | SER308 | N | 87.129 | 72.859 | 11.394 |
| 2508 | SER308 | CA | 86.035 | 72.469 | 10.512 |
| 2509 | SER308 | CB | 85.326 | 71.285 | 11.153 |
| 2510 | SER308 | OG | 86.292 | 70.261 | 11.345 |
| 2511 | SER308 | C | 85.041 | 73.616 | 10.321 |
| 2512 | SER308 | O | 83.977 | 73.41 | 9.73 |
| 2513 | LEU309 | N | 85.338 | 74.774 | 10.892 |
| 2514 | LEU309 | CA | 84.414 | 75.916 | 10.814 |
| 2515 | LEU309 | CB | 83.877 | 76.213 | 12.21 |
| 2516 | LEU309 | CG | 83.025 | 75.082 | 12.771 |
| 2517 | LEU309 | CD1 | 82.625 | 75.378 | 14.209 |
| 2518 | LEU309 | CD2 | 81.788 | 74.849 | 11.912 |
| 2519 | LEU309 | C | 85.08 | 77.187 | 10.288 |
| 2520 | LEU309 | O | 84.451 | 78.251 | 10.264 |
| 2521 | ASN310 | N | 86.354 | 77.089 | 9.95 |
| 2522 | ASN310 | CA | 87.14 | 78.264 | 9.558 |
| 2523 | ASN310 | CB | 88.615 | 77.87 | 9.489 |
| 2524 | ASN310 | CG | 88.841 | 76.726 | 8.502 |
| 2525 | ASN310 | OD1 | 88.575 | 76.853 | 7.299 |
| 2526 | ASN310 | N02 | 89.425 | 75.658 | 9.009 |
| 2527 | ASN310 | C | 86.721 | 78.879 | 8.228 |
| 2528 | ASN310 | O | 86.128 | 78.234 | 7.358 |
| 2529 | ASP311 | N | 87.234 | 80.078 | 8.014 |
| 2530 | ASP311 | CA | 87.017 | 80.838 | 6.772 |
| 2531 | ASP311 | CB | 87.177 | 82.326 | 7.089 |
| 2532 | ASP311 | CG | 88.546 | 82.608 | 7.715 |
| 2533 | ASP311 | OD1 | 88.6 | 82.705 | 8.932 |
| 2534 | ASP311 | OD2 | 89.522 | 82.647 | 6.976 |
| 2535 | ASP311 | C | 87.982 | 80.467 | 5.638 |
| 2536 | ASP311 | O | 88.142 | 81.248 | 4.695 |
| 2537 | GLN312 | N | 88.694 | 79.36 | 5.775 |
| 2538 | GLN312 | CA | 89.706 | 78.993 | 4.786 |
| 2539 | GLN312 | CB | 90.858 | 78.324 | 5.528 |
| 2540 | GLN312 | CG | 91.489 | 79.25 | 6.567 |
| 2541 | GLN312 | CD | 92.454 | 80.232 | 5.905 |
| 2542 | GLN312 | OE1 | 93.593 | 79.867 | 5.594 |
| 2543 | GLN312 | NE2 | 92.026 | 81.475 | 5.765 |
| 2544 | GLN312 | C | 89.125 | 78.029 | 3.759 |
| 2545 | GLN312 | O | 89.592 | 77.968 | 2.616 |
| 2546 | LEU313 | N | 88.075 | 77.329 | 4.151 |
| 2547 | LEU313 | CA | 87.389 | 76.449 | 3.203 |
| 2548 | LEU313 | CB | 87.452 | 75.022 | 3.737 |
| 2549 | LEU313 | CG | 86.969 | 73.997 | 2.716 |
| 2550 | LEU313 | CO1 | 87.886 | 73.971 | 1.498 |
| 2551 | LEU313 | CD2 | 86.902 | 72.611 | 3.339 |
| 2552 | LEU313 | C | 85.939 | 76.892 | 3.024 |
| 2553 | LEU313 | O | 85.143 | 76.838 | 3.966 |
| 2554 | PRO314 | N | 85.584 | 77.215 | 1.787 |
| 2555 | PRO314 | CA | 84.272 | 77.808 | 1.464 |
| 2556 | PRO314 | CB | 84.414 | 78.307 | 0.058 |
| 2557 | PRO314 | CG | 85.751 | 77.863 | −0.512 |
| 2558 | PRO314 | CD | 86.469 | 77.153 | 0.62 |
| 2559 | PRO314 | C | 83.062 | 76.863 | 1.554 |
| 2560 | PRO314 | O | 81.93 | 77.311 | 1.33 |
| 2561 | GLN315 | N | 83.278 | 75.599 | 1.879 |
| 2562 | GLN315 | CA | 82.177 | 74.646 | 2.027 |
| 2563 | GLN315 | CB | 81.639 | 74.257 | 0.653 |
| 2564 | GLN315 | CG | 82.732 | 73.871 | −0.339 |
| 2565 | GLN315 | CD | 82.079 | 73.408 | −1.634 |
| 2566 | GLN315 | OE1 | 82.749 | 73.205 | −2.653 |
| 2567 | GLN315 | NE2 | 80.767 | 73.26 | −1.577 |
| 2568 | GLN315 | C | 82.62 | 73.411 | 2.808 |
| 2569 | GLN315 | O | 83.112 | 72.429 | 2.237 |
| 2570 | HIS316 | N | 82.391 | 73.447 | 4.107 |
| 2571 | HIS316 | CA | 82.761 | 72.312 | 4.953 |
| 2572 | HIS316 | CB | 82.947 | 72.788 | 6.383 |
| 2573 | HIS316 | CG | 84.253 | 73.511 | 6.615 |
| 2574 | HIS316 | ND1 | 85.467 | 72.936 | 6.71 |
| 2575 | HIS316 | CE1 | 86.395 | 73.89 | 6.927 |
| 2576 | HIS316 | NE2 | 85.757 | 75.082 | 6.96 |
| 2577 | HIS316 | CD2 | 84.437 | 74.866 | 6.766 |
| 2578 | HIS316 | C | 81.721 | 71.202 | 4.901 |
| 2579 | HIS316 | O | 80.642 | 71.28 | 5.5 |
| 2580 | THR317 | N | 82.059 | 70.182 | 4.138 |
| 2581 | THR317 | CA | 81.231 | 68.98 | 4.03 |
| 2582 | THR317 | CB | 81.738 | 68.197 | 2.823 |
| 2583 | THR317 | OG1 | 81.674 | 69.05 | 1.688 |
| 2584 | THR317 | CG2 | 80.913 | 66.949 | 2.531 |
| 2585 | THR317 | C | 81.368 | 68.146 | 5.3 |
| 2586 | THR317 | O | 82.48 | 67.947 | 5.8 |
| 2587 | PHE318 | N | 80.247 | 67.711 | 5.846 |
| 2588 | PHE318 | CA | 80.271 | 66.885 | 7.057 |
| 2589 | PHE318 | CB | 79.684 | 67.668 | 8.222 |
| 2590 | PHE318 | CG | 80.46 | 68.921 | 8.605 |
| 2591 | PHE318 | CD1 | 79.917 | 70.176 | 8.365 |
| 2592 | PHE318 | GE1 | 80.622 | 71.316 | 8.725 |
| 2593 | PHE318 | CZ | 81.869 | 71.201 | 9.32 |
| 2594 | PHE318 | CE2 | 82.413 | 69.946 | 9.556 |
| 2595 | PHE318 | CD2 | 81.708 | 68.805 | 9.201 |
| 2596 | PHE318 | C | 79.477 | 65.598 | 6.877 |
| 2597 | PHE318 | O | 78.239 | 65.586 | 6.951 |
| 2598 | ARG319 | N | 80.206 | 64.522 | 6.647 |
| 2599 | ARG319 | CA | 79.581 | 63.204 | 6.54 |
| 2600 | ARG319 | CB | 80.305 | 62.369 | 5.495 |
| 2601 | ARG319 | CG | 80.353 | 63.087 | 4.154 |
| 2602 | ARG319 | CD | 80.774 | 62.145 | 3.032 |
| 2603 | ARG319 | NE | 82.084 | 61.526 | 3.288 |
| 2604 | ARG319 | CZ | 82.259 | 60.203 | 3.339 |
| 2605 | ARG319 | NH1 | 81.204 | 59.388 | 3.277 |
| 2606 | ARG319 | NH2 | 83.479 | 59.699 | 3.534 |
| 2607 | ARG319 | C | 79.608 | 62.478 | 7.88 |
| 2608 | ARG319 | O | 80.578 | 62.564 | 8.641 |
| 2609 | VAL320 | N | 78.503 | 61.829 | 8.188 |
| 2610 | VAL320 | CA | 78.393 | 61.032 | 9.413 |
| 2611 | VAL320 | CB | 77.323 | 61.646 | 10.311 |
| 2612 | VAL320 | CG1 | 77.124 | 60.823 | 11.577 |
| 2613 | VAL320 | CG2 | 77.677 | 63.083 | 10.675 |
| 2614 | VAL320 | C | 78.04 | 59.59 | 9.062 |
| 2615 | VAL320 | O | 76.934 | 59.303 | 8.587 |
| 2616 | 1LEA321 | N | 79.013 | 58.713 | 9.257 |
| 2617 | 1LEA321 | CA | 78.853 | 57.29 | 8.934 |
| 2618 | 1LEA321 | CB | 80.152 | 56.803 | 8.304 |
| 2619 | 1LEA321 | CG2 | 80.017 | 55.356 | 7.837 |
| 2620 | 1LEA321 | CG1 | 80.548 | 57.7 | 7.137 |
| 2621 | 1LEA321 | CD1 | 81.844 | 57.228 | 6.49 |
| 2622 | 1LEA321 | C | 78.533 | 56.444 | 10.169 |
| 2623 | 1LEA321 | O | 79.388 | 56.218 | 11.034 |
| 2624 | TRP322 | N | 77.302 | 55.969 | 10.223 |
| 2625 | TRP322 | CA | 76.856 | 55.09 | 11.313 |
| 2626 | TRP322 | CB | 75.329 | 55.048 | 11.235 |
| 2627 | TRP322 | CG | 74.543 | 54.331 | 12.322 |
| 2628 | TRP322 | CD1 | 73.333 | 53.698 | 12.124 |
| 2629 | TRP322 | NE1 | 72.909 | 53.189 | 13.308 |
| 2630 | TRP322 | CE2 | 73.783 | 53.463 | 14.295 |
| 2631 | TRP322 | CZ2 | 73.79 | 53.169 | 15.651 |
| 2632 | TRP322 | CH2 | 74.848 | 53.59 | 16.447 |
| 2633 | TRP322 | CZ3 | 75.899 | 54.31 | 15.888 |
| 2634 | TRP322 | CE3 | 75.9 | 54.606 | 14.531 |
| 2635 | TRP322 | CD2 | 74.849 | 54.188 | 13.73 |
| 2636 | TRP322 | C | 77.468 | 53.7 | 11.14 |
| 2637 | TRP322 | O | 77.334 | 53.08 | 10.081 |
| 2638 | THR323 | N | 78.152 | 53.222 | 12.167 |
| 2639 | THR323 | CA | 78.831 | 51.919 | 12.078 |
| 2640 | THR323 | 08 | 80.155 | 51.944 | 12.839 |
| 2641 | THR323 | OG1 | 79.908 | 51.984 | 14.234 |
| 2642 | THR323 | CG2 | 81.011 | 53.144 | 12.454 |
| 2643 | THR323 | C | 77.986 | 50.731 | 12.551 |
| 2644 | THR323 | O | 78.551 | 49.664 | 12.821 |
| 2645 | ALA324 | N | 76.699 | 50.932 | 12.783 |
| 2646 | ALA324 | CA | 75.818 | 49.78 | 12.999 |
| 2647 | ALA324 | CB | 74.682 | 50.146 | 13.941 |
| 2648 | ALA324 | C | 75.261 | 49.363 | 11.649 |
| 2649 | ALA324 | O | 75.547 | 48.279 | 11.128 |
| 2650 | GLY325 | N | 74.453 | 50.251 | 11.104 |
| 2651 | GLY325 | CA | 74.069 | 50.174 | 9.696 |
| 2652 | GLY325 | C | 74.764 | 51.357 | 9.043 |
| 2653 | GLY325 | O | 74.565 | 52.486 | 9.504 |
| 2654 | ASP326 | N | 75.535 | 51.102 | 7.994 |
| 2655 | ASP326 | CA | 76.438 | 52.107 | 7.385 |
| 2656 | ASP326 | CB | 77.444 | 51.389 | 6.492 |
| 2657 | ASP326 | CG | 78.39 | 50.525 | 7.326 |
| 2658 | ASP326 | OD1 | 79.453 | 51.023 | 7.668 |
| 2659 | ASP326 | OD2 | 78.082 | 49.354 | 7.5 |
| 2660 | ASP326 | C | 75.76 | 53.216 | 6.577 |
| 2661 | ASP326 | O | 75.896 | 53.297 | 5.351 |
| 2662 | VAL327 | N | 75.113 | 54.114 | 7.297 |
| 2663 | VAL327 | CA | 74.469 | 55.282 | 6.706 |
| 2664 | VAL327 | CB | 73.305 | 55.671 | 7.608 |
| 2665 | VAL327 | CG1 | 72.549 | 56.877 | 7.069 |
| 2666 | VAL327 | CG2 | 72.362 | 54.494 | 7.782 |
| 2667 | VAL327 | C | 75.463 | 56.424 | 6.642 |
| 2668 | VAL327 | O | 76.061 | 56.777 | 7.661 |
| 2669 | GLN328 | N | 75.675 | 56.948 | 5.448 |
| 2670 | GLN328 | CA | 76.599 | 58.068 | 5.272 |
| 2671 | GLN328 | CB | 77.55 | 57.723 | 4.135 |
| 2672 | GLN328 | CG | 78.262 | 56.408 | 4.429 |
| 2673 | GLN328 | CD | 79.182 | 56.022 | 3.279 |
| 2674 | GLN328 | OE1 | 79.176 | 56.651 | 2.216 |
| 2675 | GLN328 | NE2 | 79.955 | 54.975 | 3.506 |
| 2676 | GLN328 | C | 75.839 | 59.352 | 4.96 |
| 2677 | GLN328 | O | 75.788 | 59.803 | 3.81 |
| 2678 | LYS329 | N | 75.256 | 59.932 | 5.995 |
| 2679 | LYS329 | CA | 74.521 | 61.19 | 5.83 |
| 2680 | LYS329 | CB | 73.659 | 61.431 | 7.059 |
| 2681 | LYS329 | CG | 72.332 | 60.692 | 6.97 |
| 2682 | LYS329 | CD | 71.494 | 61.233 | 5.818 |
| 2683 | LYS329 | CE | 70.104 | 60.61 | 5.791 |
| 2684 | LYS329 | NZ | 70.178 | 59.15 | 5.64 |
| 2685 | LYS329 | C | 75.498 | 62.338 | 5.622 |
| 2686 | LYS329 | O | 76.609 | 62.312 | 6.157 |
| 2687 | GLU330 | N | 75.113 | 63.313 | 4.819 |
| 2688 | GLU330 | CA | 76.046 | 64.401 | 4.504 |
| 2689 | GLU330 | CB | 76.628 | 64.131 | 3.121 |
| 2690 | GLU330 | CG | 77.58 | 65.24 | 2.685 |
| 2691 | GLU330 | CD | 78.049 | 64.999 | 1.255 |
| 2692 | GLU330 | OE1 | 79.085 | 64.369 | 1.096 |
| 2693 | GLU330 | OE2 | 77.377 | 65.467 | 0.347 |
| 2694 | GLU330 | C | 75.397 | 65.782 | 4.506 |
| 2695 | GLU330 | O | 74.627 | 66.121 | 3.601 |
| 2696 | CYS331 | N | 75.759 | 66.585 | 5.491 |
| 2697 | CYS331 | CA | 75.379 | 68.002 | 5.476 |
| 2698 | CYS331 | CB | 74.959 | 68.449 | 6.872 |
| 2699 | CYS331 | 5G | 76.146 | 68.153 | 8.198 |
| 2700 | CYS331 | C | 76.56 | 68.819 | 4.957 |
| 2701 | CYS331 | O | 77.671 | 68.29 | 4.827 |
| 2702 | VAL332 | N | 76.298 | 70.036 | 4.516 |
| 2703 | VAL332 | CA | 77.398 | 70.876 | 4.021 |
| 2704 | VAL332 | CB | 77.485 | 70.762 | 2.497 |
| 2705 | VAL332 | CG1 | 76.152 | 71.042 | 1.811 |
| 2706 | VAL332 | CG2 | 78.597 | 71.634 | 1.921 |
| 2707 | VAL332 | C | 77.253 | 72.327 | 4.483 |
| 2708 | VAL332 | O | 76.302 | 73.041 | 4.135 |
| 2709 | LEU333 | N | 78.228 | 72.755 | 5.264 |
| 2710 | LEU333 | CA | 78.225 | 74.107 | 5.815 |
| 2711 | LEU333 | CB | 78.87 | 74.04 | 7.19 |
| 2712 | LEU333 | CG | 78.602 | 75.292 | 8.01 |
| 2713 | LEU333 | CD1 | 77.107 | 75.559 | 8.108 |
| 2714 | LEU333 | C02 | 79.203 | 75.142 | 9.399 |
| 2715 | LEU333 | C | 78.991 | 75.064 | 4.904 |
| 2716 | LEU333 | O | 80.221 | 75.197 | 4.984 |
| 2717 | LEU334 | N | 78.243 | 75.681 | 4.006 |
| 2718 | LEU334 | CA | 78.797 | 76.665 | 3.068 |
| 2719 | LEU334 | CB | 77.698 | 77.069 | 2.091 |
| 2720 | LEU334 | CG | 77.111 | 75.871 | 1.354 |
| 2721 | LEU334 | CD1 | 75.824 | 76.254 | 0.633 |
| 2722 | LEU334 | CD2 | 78.118 | 75.268 | 0.383 |
| 2723 | LEU334 | C | 79.263 | 77.906 | 3.817 |
| 2724 | LEU334 | O | 78.781 | 78.186 | 4.921 |
| 2725 | LYS335 | N | 80.216 | 78.616 | 3.241 |
| 2726 | LYS335 | CA | 80.699 | 79.86 | 3.848 |
| 2727 | LYS335 | CB | 81.797 | 80.455 | 2.972 |
| 2728 | LYS335 | CG | 81.324 | 80.67 | 1.539 |
| 2729 | LYS335 | CD | 82.408 | 81.31 | 0.684 |
| 2730 | LYS335 | CE | 81.949 | 81.47 | −0.76 |
| 2731 | LYS335 | NZ | 83.018 | 82.05 | −1.588 |
| 2732 | LYS335 | C | 79.557 | 80.857 | 4.023 |
| 2733 | LYS335 | O | 78.725 | 81.054 | 3.131 |
| 2734 | GLY336 | N | 79.415 | 81.319 | 5.252 |
| 2735 | GLY336 | CA | 78.353 | 82.27 | 5.58 |
| 2736 | GLY336 | C | 77.187 | 81.594 | 6.301 |
| 2737 | GLY336 | O | 76.427 | 82.255 | 7.019 |
| 2738 | ARG337 | N | 77.028 | 80.299 | 6.083 |
| 2739 | ARG337 | CA | 75.944 | 79.565 | 6.731 |
| 2740 | ARG337 | CB | 75.735 | 78.232 | 6.024 |
| 2741 | ARG337 | CG | 75.365 | 78.432 | 4.561 |
| 2742 | ARG337 | CD | 74.039 | 79.168 | 4.414 |
| 2743 | ARG337 | NE | 73.756 | 79.443 | 2.998 |
| 2744 | ARG337 | CZ | 73.41 | 80.652 | 2.553 |
| 2745 | ARG337 | NH1 | 73.293 | 81.67 | 3.409 |
| 2746 | ARG337 | NH2 | 73.17 | 80.841 | 1.253 |
| 2747 | ARG337 | C | 76.289 | 79.325 | 8.19 |
| 2748 | ARG337 | O | 77.355 | 78.8 | 8.529 |
| 2749 | GLN338 | N | 75.374 | 79.726 | 9.051 |
| 2750 | GLN338 | CA | 75.571 | 79.535 | 10.484 |
| 2751 | GLN338 | CB | 74.838 | 80.664 | 11.191 |
| 2752 | GLN338 | CG | 75.341 | 82.022 | 10.721 |
| 2753 | GLN338 | CD | 74.497 | 83.125 | 11.349 |
| 2754 | GLN338 | OE1 | 73.733 | 83.809 | 10.658 |
| 2755 | GLN338 | NE2 | 74.591 | 83.237 | 12.662 |
| 2756 | GLN338 | C | 75.01 | 78.195 | 10.943 |
| 2757 | GLN338 | O | 75.391 | 77.685 | 12.006 |
| 2758 | GLU339 | N | 74.146 | 77.62 | 10.119 |
| 2759 | GLU339 | CA | 73.51 | 76.327 | 10.413 |
| 2760 | GLU339 | CB | 72.156 | 76.589 | 11.07 |
| 2761 | GLU339 | CG | 72.293 | 77.182 | 12.471 |
| 2762 | GLU339 | CD | 70.923 | 77.452 | 13.078 |
| 2763 | GLU339 | OE1 | 70.351 | 78.481 | 12.745 |
| 2764 | GLU339 | OE2 | 70.449 | 76.601 | 13.817 |
| 2765 | GLU339 | C | 73.312 | 75.515 | 9.13 |
| 2766 | GLU339 | O | 72.672 | 75.987 | 8.182 |
| 2767 | GLY340 | N | 73.838 | 74.302 | 9.12 |
| 2768 | GLY340 | CA | 73.742 | 73.425 | 7.941 |
| 2769 | GLY340 | C | 73.234 | 72.025 | 8.296 |
| 2770 | GLY340 | O | 73.964 | 71.184 | 8.837 |
| 2771 | TRP341 | N | 71.989 | 71.769 | 7.945 |
| 2772 | TRP341 | CA | 71.357 | 70.492 | 8.295 |
| 2773 | TRP341 | CB | 69.985 | 70.768 | 8.902 |
| 2774 | TRP341 | CG | 69.062 | 71.626 | 8.061 |
| 2775 | TRP341 | CD1 | 68.229 | 71.199 | 7.051 |
| 2776 | TRP341 | NE1 | 67.576 | 72.278 | 6.548 |
| 2777 | TRP341 | CE2 | 67.936 | 73.407 | 7.186 |
| 2778 | TRP341 | CZ2 | 67.564 | 74.734 | 7.034 |
| 2779 | TRP341 | CH2 | 68.12 | 75.705 | 7.858 |
| 2780 | TRP341 | CZ3 | 69.047 | 75.353 | 8.834 |
| 2781 | TRP341 | CE3 | 69.424 | 74.026 | 8.994 |
| 2782 | TRP341 | CD2 | 68.875 | 73.055 | 8.171 |
| 2783 | TRP341 | C | 71.224 | 69.53 | 7.116 |
| 2784 | TRP341 | O | 71.519 | 69.866 | 5.963 |
| 2785 | CYS342 | N | 70.88 | 68.302 | 7.465 |
| 2786 | CYS342 | CA | 70.591 | 67.233 | 6.497 |
| 2787 | CYS342 | CB | 71.858 | 66.44 | 6.209 |
| 2788 | CYS342 | SG | 71.677 | 65.093 | 5.019 |
| 2789 | CYS342 | C | 69.526 | 66.305 | 7.08 |
| 2790 | CYS342 | O | 69.838 | 65.288 | 7.718 |
| 2791 | ARG343 | N | 68.276 | 66.687 | 6.88 |
| 2792 | ARG343 | CA | 67.147 | 65.981 | 7.498 |
| 2793 | ARG343 | CB | 66.281 | 67.039 | 8.178 |
| 2794 | ARG343 | CG | 65.123 | 66.42 | 8.949 |
| 2795 | ARG343 | CD | 64.16 | 67.468 | 9.484 |
| 2796 | ARG343 | NE | 63.042 | 66.812 | 10.175 |
| 2797 | ARG343 | CZ | 61.85 | 66.603 | 9.612 |
| 2798 | ARG343 | NH1 | 61.607 | 67.049 | 8.377 |
| 2799 | ARG343 | NH2 | 60.89 | 65.983 | 10.3 |
| 2800 | ARG343 | C | 66.291 | 65.19 | 6.501 |
| 2801 | ARG343 | O | 65.952 | 65.676 | 5.417 |
| 2802 | ASP344 | N | 65.973 | 63.964 | 6.883 |
| 2803 | ASP344 | CA | 64.991 | 63.142 | 6.171 |
| 2804 | ASP344 | CB | 65.065 | 61.717 | 6.715 |
| 2805 | ASP344 | CG | 66.4 | 61.059 | 6.388 |
| 2806 | ASP344 | OD1 | 66.826 | 61.23 | 5.253 |
| 2807 | ASP344 | 0D2 | 66.783 | 60.177 | 7.146 |
| 2808 | ASP344 | C | 63.585 | 63.666 | 6.445 |
| 2809 | ASP344 | O | 63.187 | 63.803 | 7.607 |
| 2810 | SER345 | N | 62.833 | 63.93 | 5.392 |
| 2811 | SER345 | CA | 61.455 | 64.379 | 5.581 |
| 2812 | SER345 | CB | 60.942 | 65.099 | 4.337 |
| 2813 | SER345 | OG | 60.414 | 64.125 | 3.444 |
| 2814 | SER345 | C | 60.566 | 63.179 | 5.861 |
| 2815 | SER345 | O | 60.749 | 62.087 | 5.304 |
| 2816 | THR346 | N | 59.503 | 63.44 | 6.598 |
| 2817 | THR346 | CA | 58.547 | 62.387 | 6.931 |
| 2818 | THR346 | CB | 57.641 | 62.895 | 8.046 |
| 2819 | THR346 | OG1 | 56.845 | 63.966 | 7.554 |
| 2820 | THR346 | CG2 | 58.451 | 63.406 | 9.231 |
| 2821 | THR346 | C | 57.695 | 61.992 | 5.732 |
| 2822 | THR346 | O | 57.308 | 60.82 | 5.624 |
| 2823 | THR347 | N | 57.624 | 62.884 | 4.756 |
| 2824 | THR347 | CA | 56.861 | 62.628 | 3.542 |
| 2825 | THR347 | CB | 56.594 | 63.963 | 2.854 |
| 2826 | THR347 | OG1 | 55.892 | 64.8 | 3.764 |
| 2827 | THR347 | CG2 | 55.738 | 63.799 | 1.603 |
| 2828 | THR347 | C | 57.595 | 61.701 | 2.579 |
| 2829 | THR347 | O | 57.116 | 60.587 | 2.334 |
| 2830 | ASP348 | N | 58.813 | 62.056 | 2.191 |
| 2831 | ASP348 | CA | 59.478 | 61.34 | 1.1 |
| 2832 | ASP348 | CB | 60.322 | 62.349 | 0.326 |
| 2833 | ASP348 | CG | 59.494 | 63.58 | −0.036 |
| 2834 | ASP348 | OD1 | 58.651 | 63.463 | −0.913 |
| 2835 | ASP348 | OD2 | 59.618 | 64.573 | 0.671 |
| 2836 | ASP348 | C | 60.389 | 60.221 | 1.593 |
| 2837 | ASP348 | O | 61.026 | 59.531 | 0.791 |
| 2838 | GLU349 | N | 60.563 | 60.132 | 2.9 |
| 2839 | GLU349 | CA | 61.417 | 59.073 | 3.439 |
| 2840 | GLU349 | CB | 62.563 | 59.668 | 4.259 |
| 2841 | GLU349 | CG | 63.789 | 60.04 | 3.415 |
| 2842 | GLU349 | CD | 63.561 | 61.254 | 2.512 |
| 2843 | GLU349 | OE1 | 62.904 | 62.188 | 2.963 |
| 2844 | GLU349 | OE2 | 64.113 | 61.273 | 1.423 |
| 2845 | GLU349 | C | 60.617 | 58.081 | 4.273 |
| 2846 | GLU349 | O | 61.183 | 57.063 | 4.697 |
| 2847 | GLN350 | N | 59.313 | 58.315 | 4.379 |
| 2848 | GLN350 | CA | 58.415 | 57.475 | 5.187 |
| 2849 | GLN350 | CB | 58.423 | 56.061 | 4.621 |
| 2850 | GLN350 | CG | 58.036 | 56.037 | 3.153 |
| 2851 | GLN350 | CD | 58.521 | 54.741 | 2.52 |
| 2852 | GLN350 | OE1 | 57.727 | 53.957 | 1.975 |
| 2853 | GLN350 | NE2 | 59.814 | 54.509 | 2.667 |
| 2854 | GLN350 | C | 58.862 | 57.443 | 6.644 |
| 2855 | GLN350 | O | 59.626 | 56.562 | 7.059 |
| 2856 | LEU351 | N | 58.432 | 58.437 | 7.403 |
| 2857 | LEU351 | CA | 58.807 | 58.482 | 8.825 |
| 2858 | LEU351 | CB | 59.121 | 59.913 | 9.224 |
| 2859 | LEU351 | CG | 60.588 | 60.116 | 9.574 |
| 2860 | LEU351 | CD1 | 60.982 | 59.146 | 10.676 |
| 2861 | LEU351 | CD2 | 61.493 | 59.96 | 8.356 |
| 2862 | LEU351 | C | 57.697 | 57.966 | 9.731 |
| 2863 | LEU351 | O | 57.924 | 57.694 | 10.915 |
| 2864 | PHE352 | N | 56.503 | 57.877 | 9.173 |
| 2865 | PHE352 | CA | 55.345 | 57.353 | 9.902 |
| 2866 | PHE352 | CB | 54.837 | 58.377 | 10.921 |
| 2867 | PHE352 | CG | 54.688 | 59.82 | 10.436 |
| 2868 | PHE352 | CD1 | 53.766 | 60.151 | 9.451 |
| 2869 | PHE352 | CE1 | 53.645 | 61.467 | 9.026 |
| 2870 | PHE352 | CZ | 54.437 | 62.455 | 9.595 |
| 2871 | PHE352 | CE2 | 55.348 | 62.128 | 10.589 |
| 2872 | PHE352 | CD2 | 55.472 | 60.812 | 11.011 |
| 2873 | PHE352 | C | 54.248 | 56.963 | 8.923 |
| 2874 | PHE352 | O | 53.099 | 56.724 | 9.312 |
| 2875 | ARG353 | N | 54.63 | 56.865 | 7.661 |
| 2876 | ARG353 | CA | 53.661 | 56.562 | 6.608 |
| 2877 | ARG353 | CB | 52.87 | 57.838 | 6.294 |
| 2878 | ARG353 | CG | 51.652 | 57.607 | 5.394 |
| 2879 | ARG353 | CD | 51.974 | 57.749 | 3.91 |
| 2880 | ARG353 | NE | 50.811 | 57.414 | 3.074 |
| 2881 | ARG353 | CZ | 50.799 | 57.584 | 1.751 |
| 2882 | ARG353 | NH1 | 51.842 | 58.152 | 1.143 |
| 2883 | ARG353 | NH2 | 49.724 | 57.237 | 1.04 |
| 2884 | ARG353 | C | 54.382 | 56.03 | 5.374 |
| 2885 | ARG353 | O | 55.081 | 56.779 | 4.677 |
| 2886 | CYS354 | N | 54.256 | 54.728 | 5.176 |
| 2887 | CYS354 | CA | 54.743 | 54.07 | 3.961 |
| 2888 | CYS354 | CB | 54.431 | 52.581 | 4.074 |
| 2889 | CYS354 | SG | 54.646 | 51.606 | 2.567 |
| 2890 | CYS354 | C | 54.037 | 54.639 | 2.737 |
| 2891 | CYS354 | O | 52.808 | 54.766 | 2.716 |
| 2892 | GLU355 | N | 54.818 | 54.979 | 1.727 |
| 2893 | GLU355 | CA | 54.251 | 55.537 | 0.498 |
| 2894 | GLU355 | CB | 55.334 | 56.37 | −0.179 |
| 2895 | GLU355 | CG | 55.695 | 57.55 | 0.726 |
| 2896 | GLU355 | CD | 56.902 | 58.322 | 0.2 |
| 2897 | GLU355 | OE1 | 56.733 | 59.068 | −0.754 |
| 2898 | GLU355 | OE2 | 57.982 | 58.126 | 0.744 |
| 2899 | GLU355 | C | 53.716 | 54.409 | −0.383 |
| 2900 | GLU355 | O | 54.47 | 53.672 | −1.028 |
| 2901 | LEU356 | N | 52.398 | 54.291 | −0.371 |
| 2902 | LEU356 | CA | 51.706 | 53.151 | −0.987 |
| 2903 | LEU356 | CB | 50.222 | 53.198 | −0.632 |
| 2904 | LEU356 | CG | 49.906 | 53.466 | 0.834 |
| 2905 | LEU356 | CD1 | 48.395 | 53.548 | 1.003 |
| 2906 | LEU356 | CD2 | 50.484 | 52.409 | 1.768 |
| 2907 | LEU356 | C | 51.768 | 53.16 | −2.508 |
| 2908 | LEU356 | O | 51.848 | 54.213 | −3.149 |
| 2909 | SER357 | N | 51.722 | 51.965 | −3.069 |
| 2910 | SER357 | CA | 51.506 | 51.821 | −4.511 |
| 2911 | SER357 | CB | 51.815 | 50.395 | −4.934 |
| 2912 | SER357 | OG | 50.721 | 49.6 | −4.493 |
| 2913 | SER357 | C | 50.031 | 52.07 | −4.789 |
| 2914 | SER357 | O | 49.215 | 52.008 | −3.862 |
| 2915 | VAL358 | N | 49.667 | 52.128 | −6.059 |
| 2916 | VAL358 | CA | 48.256 | 52.34 | −6.413 |
| 2917 | VAL358 | CB | 48.181 | 52.647 | −7.904 |
| 2918 | VAL358 | CG1 | 46.738 | 52.839 | −8.359 |
| 2919 | VAL358 | CG2 | 49.021 | 53.872 | −8.252 |
| 2920 | VAL358 | C | 47.409 | 51.106 | −6.098 |
| 2921 | VAL358 | O | 46.312 | 51.241 | −5.547 |
| 2922 | GLU359 | N | 48.047 | 49.948 | −6.138 |
| 2923 | GLU359 | CA | 47.387 | 48.681 | −5.809 |
| 2924 | GLU359 | CB | 48.292 | 47.496 | −6.178 |
| 2925 | GLU359 | CG | 48.511 | 47.274 | −7.68 |
| 2926 | GLU359 | CD | 49.659 | 48.115 | −8.241 |
| 2927 | GLU359 | OE1 | 50.339 | 48.749 | −7.439 |
| 2928 | GLU359 | OE2 | 49.679 | 48.302 | −9.447 |
| 2929 | GLU359 | C | 47.091 | 48.602 | −4.315 |
| 2930 | GLU359 | O | 45.937 | 48.366 | −3.931 |
| 2931 | LYS360 | N | 48.052 | 49.021 | −3.504 |
| 2932 | LYS360 | CA | 47.85 | 49.002 | −2.054 |
| 2933 | LYS360 | CB | 49.21 | 49.147 | −1.387 |
| 2934 | LYS360 | CG | 49.128 | 48.929 | 0.118 |
| 2935 | LYS360 | CD | 50.512 | 48.968 | 0.756 |
| 2936 | LYS360 | CE | 50.435 | 48.743 | 2.262 |
| 2937 | LYS360 | NZ | 51.764 | 48.885 | 2.881 |
| 2938 | LY3360 | C | 46.916 | 50.117 | −1.583 |
| 2939 | LYS360 | O | 46.097 | 49.873 | −0.69 |
| 2940 | SER361 | N | 46.839 | 51.197 | −2.342 |
| 2941 | SER361 | CA | 45.907 | 52.273 | −2 |
| 2942 | SER361 | CB | 46.299 | 53.526 | −2.774 |
| 2943 | SER361 | OG | 47.621 | 53.884 | −2.401 |
| 2944 | SER361 | C | 44.473 | 51.899 | −2.358 |
| 2945 | SER361 | O | 43.564 | 52.149 | −1.557 |
| 2946 | THR362 | N | 44.318 | 51.073 | −3.379 |
| 2947 | THR362 | CA | 42.983 | 50.644 | −3.799 |
| 2948 | THR362 | CB | 43.086 | 50.014 | −5.184 |
| 2949 | THR362 | OG1 | 43.541 | 51.012 | −6.087 |
| 2950 | THR362 | CG2 | 41.732 | 49.515 | −5.68 |
| 2951 | THR362 | C | 42.39 | 49.636 | −2.824 |
| 2952 | THR362 | O | 41.261 | 49.839 | −2.358 |
| 2953 | VAL363 | N | 43.216 | 48.736 | −2.314 |
| 2954 | VAL363 | CA | 42.685 | 47.755 | −1.364 |
| 2955 | VAL363 | CB | 43.541 | 46.488 | −1.391 |
| 2956 | VAL363 | CG1 | 45.012 | 46.782 | −1.145 |
| 2957 | VAL363 | CG2 | 43.032 | 45.439 | −0.407 |
| 2958 | VAL363 | C | 42.578 | 48.333 | 0.049 |
| 2959 | VAL363 | O | 41.624 | 47.985 | 0.758 |
| 2960 | LEU364 | N | 43.309 | 49.401 | 0.326 |
| 2961 | LEU364 | CA | 43.186 | 50.061 | 1.629 |
| 2962 | LEU364 | CB | 44.437 | 50.89 | 1.898 |
| 2963 | LEU364 | CG | 45.393 | 50.244 | 2.901 |
| 2964 | LEU364 | CD1 | 45.868 | 48.859 | 2.474 |
| 2965 | LEU364 | CD2 | 46.589 | 51.153 | 3.152 |
| 2966 | LEU364 | C | 41.958 | 50.964 | 1.663 |
| 2967 | LEU364 | O | 41.223 | 50.952 | 2.66 |
| 2968 | GLN365 | N | 41.583 | 51.481 | 0.503 |
| 2969 | GLN365 | CA | 40.363 | 52.282 | 0.403 |
| 2970 | GLN365 | CB | 40.404 | 53.074 | −0.899 |
| 2971 | GLN365 | CG | 39.306 | 54.131 | −0.963 |
| 2972 | GLN365 | CD | 39.646 | 55.295 | −0.035 |
| 2973 | GLN365 | OE1 | 40.748 | 55.85 | −0.104 |
| 2974 | GLN365 | NE2 | 38.698 | 55.663 | 0.809 |
| 2975 | GLN365 | C | 39.131 | 51.381 | 0.398 |
| 2976 | GLN365 | O | 38.118 | 51.727 | 1.02 |
| 2977 | SER366 | N | 39.296 | 50.157 | −0.079 |
| 2978 | SER366 | CA | 38.203 | 49.181 | −0.04 |
| 2979 | SER366 | CB | 38.544 | 48.025 | −0.967 |
| 2980 | SER366 | OG | 37.551 | 47.028 | −0.773 |
| 2981 | SER366 | C | 37.983 | 48.632 | 1.364 |
| 2982 | SER366 | O | 36.83 | 48.505 | 1.796 |
| 2983 | GLU367 | N | 39.054 | 48.538 | 2.136 |
| 2984 | GLU367 | CA | 38.936 | 48.114 | 3.534 |
| 2985 | GLU367 | CB | 40.324 | 47.727 | 4.037 |
| 2986 | GLU367 | CG | 40.864 | 46.5 | 3.312 |
| 2987 | GLU367 | CD | 42.364 | 46.363 | 3.553 |
| 2988 | GLU367 | OE1 | 43.015 | 47.397 | 3.63 |
| 2989 | GLU367 | OE2 | 42.856 | 45.244 | 3.526 |
| 2990 | GLU367 | C | 38.379 | 49.243 | 4.394 |
| 2991 | GLU367 | O | 37.537 | 48.989 | 5.263 |
| 2992 | LEU368 | N | 38.634 | 50.472 | 3.974 |
| 2993 | LEU368 | CA | 38.104 | 51.643 | 4.672 |
| 2994 | LEU368 | CB | 38.827 | 52.869 | 4.12 |
| 2995 | LEU368 | CG | 38.433 | 54.137 | 4.86 |
| 2996 | LEU368 | CO1 | 38.702 | 53.986 | 6.348 |
| 2997 | LEU368 | CD2 | 39.175 | 55.347 | 4.308 |
| 2998 | LEU368 | C | 36.601 | 51.787 | 4.453 |
| 2999 | LEU368 | O | 35.853 | 51.89 | 5.435 |
| 3000 | GLU369 | N | 36.15 | 51.502 | 3.24 |
| 3001 | GLU369 | CA | 34.716 | 51.609 | 2.948 |
| 3002 | GLU369 | CB | 34.467 | 51.537 | 1.444 |
| 3003 | GLU369 | CG | 35.245 | 52.579 | 0.652 |
| 3004 | GLU369 | CD | 34.964 | 53.994 | 1.145 |
| 3005 | GLU369 | OE1 | 33.806 | 54.382 | 1.149 |
| 3006 | GLU369 | OE2 | 35.941 | 54.713 | 1.315 |
| 3007 | GLU369 | C | 33.951 | 50.465 | 3.593 |
| 3008 | GLU369 | O | 32.907 | 50.702 | 4.213 |
| 3009 | SER370 | N | 34.595 | 49.316 | 3.692 |
| 3010 | SER370 | CA | 33.934 | 48.154 | 4.273 |
| 3011 | SER370 | CB | 34.606 | 46.911 | 3.716 |
| 3012 | SER370 | OG | 34.433 | 46.935 | 2.305 |
| 3013 | SER370 | C | 33.947 | 48.159 | 5.801 |
| 3014 | SER370 | O | 32.996 | 47.648 | 6.405 |
| 3015 | CYS371 | N | 34.828 | 48.941 | 6.407 |
| 3016 | CYS371 | CA | 34.771 | 49.096 | 7.862 |
| 3017 | CYS371 | CB | 36.149 | 49.437 | 8.408 |
| 3018 | CYS371 | SG | 36.71 | 48.37 | 9.751 |
| 3019 | CYS371 | C | 33.768 | 50.179 | 8.245 |
| 3020 | CYS371 | O | 33.097 | 50.046 | 9.277 |
| 3021 | LYS372 | N | 33.469 | 51.065 | 7.307 |
| 3022 | LYS372 | CA | 32.377 | 52.02 | 7.516 |
| 3023 | LYS372 | CB | 32.531 | 53.174 | 6.533 |
| 3024 | LYS372 | OG | 33.813 | 53.949 | 6.804 |
| 3025 | LYS372 | CD | 34.021 | 55.07 | 5.796 |
| 3026 | LYS372 | CE | 35.283 | 55.86 | 6.119 |
| 3027 | LYS372 | NZ | 35.517 | 56.919 | 5.123 |
| 3028 | LYS372 | C | 31.029 | 51.338 | 7.303 |
| 3029 | LYS372 | O | 30.096 | 51.566 | 8.083 |
| 3030 | GLU373 | N | 31.028 | 50.308 | 6.473 |
| 3031 | GLU373 | CA | 29.825 | 49.493 | 6.285 |
| 3032 | GLU373 | CB | 29.989 | 48.674 | 5.01 |
| 3033 | GLU373 | CG | 30.057 | 49.582 | 3.787 |
| 3034 | GLU373 | CD | 30.41 | 48.774 | 2.541 |
| 3035 | GLU373 | OE1 | 31.594 | 48.677 | 2.237 |
| 3036 | GLU373 | OE2 | 29.494 | 48.273 | 1.906 |
| 3037 | GLU373 | C | 29.588 | 48.563 | 7.473 |
| 3038 | GLU373 | O | 28.439 | 48.428 | 7.91 |
| 3039 | LEU374 | N | 30.657 | 48.174 | 8.15 |
| 3040 | LEU374 | CA | 30.506 | 47.379 | 9.37 |
| 3041 | LEU374 | CB | 31.813 | 46.664 | 9.673 |
| 3042 | LEU374 | CG | 31.612 | 45.162 | 9.838 |
| 3043 | LEU374 | CO1 | 32.918 | 44.498 | 10.253 |
| 3044 | LEU374 | CD2 | 30.517 | 44.846 | 10.85 |
| 3045 | LEU374 | C | 30.123 | 48.257 | 10.558 |
| 3046 | LEU374 | O | 29.314 | 47.816 | 11.381 |
| 3047 | GLN375 | N | 30.449 | 49.538 | 10.493 |
| 3048 | GLN375 | CA | 29.968 | 50.497 | 11.495 |
| 3049 | GLN375 | CB | 30.783 | 51.776 | 11.35 |
| 3050 | GLN375 | CG | 30.289 | 52.858 | 12.301 |
| 3051 | GLN375 | CD | 30.87 | 54.208 | 11.905 |
| 3052 | GLN375 | OE1 | 31.523 | 54.341 | 10.862 |
| 3053 | GLN375 | NE2 | 30.607 | 55.201 | 12.737 |
| 3054 | GLN375 | C | 28.489 | 50.83 | 11.28 |
| 3055 | GLN375 | O | 27.755 | 51.059 | 12.248 |
| 3056 | GLU376 | N | 28.017 | 50.64 | 10.059 |
| 3057 | GLU376 | CA | 26.594 | 50.819 | 9.754 |
| 3058 | GLU376 | CB | 26.455 | 51.072 | 8.258 |
| 3059 | GLU376 | CG | 27.144 | 52.365 | 7.842 |
| 3060 | GLU376 | CD | 27.224 | 52.44 | 6.32 |
| 3061 | GLU376 | OE1 | 26.191 | 52.286 | 5.686 |
| 3062 | GLU376 | OE2 | 28.333 | 52.56 | 5.811 |
| 3063 | GLU376 | C | 25.761 | 49.591 | 10.128 |
| 3064 | GLU376 | O | 24.531 | 49.684 | 10.207 |
| 3065 | LEU377 | N | 26.418 | 48.472 | 10.391 |
| 3066 | LEU377 | CA | 25.709 | 47.277 | 10.855 |
| 3067 | LEU377 | CB | 26.354 | 46.054 | 10.213 |
| 3068 | LEU377 | CG | 26.279 | 46.11 | 8.691 |
| 3069 | LEU377 | CD1 | 27.122 | 45.007 | 8.062 |
| 3070 | LEU377 | CD2 | 24.834 | 46.039 | 8.203 |
| 3071 | LEU377 | C | 25.82 | 47.164 | 12.37 |
| 3072 | LEU377 | O | 24.919 | 46.656 | 13.049 |
| 3073 | GLU378 | N | 26.957 | 47.605 | 12.877 |
| 3074 | GLU378 | CA | 27.215 | 47.67 | 14.316 |
| 3075 | GLU378 | CB | 28.193 | 46.562 | 14.711 |
| 3076 | GLU378 | CG | 27.663 | 45.153 | 14.464 |
| 3077 | GLU378 | CD | 28.728 | 44.126 | 14.849 |
| 3078 | GLU378 | OE1 | 29.898 | 44.431 | 14.673 |
| 3079 | GLU378 | OE2 | 28.353 | 43.04 | 15.278 |
| 3080 | GLU378 | C | 27.872 | 49.003 | 14.653 |
| 3081 | GLU378 | O | 29.107 | 49.069 | 14.708 |
| 3082 | PRO379 | N | 27.078 | 49.97 | 15.091 |
| 3083 | PRO379 | CA | 27.594 | 51.326 | 15.356 |
| 3084 | PRO379 | CB | 26.368 | 52.177 | 15.49 |
| 3085 | PRO379 | CG | 25.134 | 51.287 | 15.512 |
| 3086 | PRO379 | CD | 25.629 | 49.868 | 15.289 |
| 3087 | PRO379 | C | 28.459 | 51.432 | 16.618 |
| 3088 | PRO379 | O | 29.132 | 52.447 | 16.831 |
| 3089 | GLU380 | N | 28.463 | 50.382 | 17.423 |
| 3090 | GLU380 | CA | 29.303 | 50.319 | 18.617 |
| 3091 | GLU380 | CB | 28.471 | 49.777 | 19.771 |
| 3092 | GLU380 | CG | 27.321 | 50.715 | 20.115 |
| 3093 | GLU380 | CD | 26.455 | 50.095 | 21.205 |
| 3094 | GLU380 | OE1 | 26.65 | 50.437 | 22.362 |
| 3095 | GLU380 | OE2 | 25.589 | 49.307 | 20.848 |
| 3096 | GLU380 | C | 30.534 | 49.434 | 18.42 |
| 3097 | GLU380 | O | 31.172 | 49.066 | 19.413 |
| 3098 | ASN381 | N | 30.802 | 48.991 | 17.2 |
| 3099 | ASN381 | CA | 31.996 | 48.168 | 16.992 |
| 3100 | ASN381 | CB | 31.838 | 47.299 | 15.745 |
| 3101 | ASN381 | CG | 33.053 | 46.383 | 15.596 |
| 3102 | ASN381 | OD1 | 34.117 | 46.832 | 15.151 |
| 3103 | ASN381 | ND2 | 32.922 | 45.147 | 16.041 |
| 3104 | ASN381 | C | 33.225 | 49.067 | 16.892 |
| 3105 | ASN381 | O | 33.609 | 49.542 | 15.814 |
| 3106 | LYS382 | N | 33.958 | 49.089 | 17.993 |
| 3107 | LYS382 | CA | 35.127 | 49.958 | 18.129 |
| 3108 | LYS382 | CB | 35.398 | 50.128 | 19.619 |
| 3109 | LYS382 | CG | 35.696 | 48.803 | 20.31 |
| 3110 | LYS382 | CD | 35.811 | 48.991 | 21.816 |
| 3111 | LYS382 | CE | 36.287 | 47.716 | 22.498 |
| 3112 | LY3382 | NZ | 37.641 | 47.369 | 22.042 |
| 3113 | LYS382 | C | 36.372 | 49.438 | 17.408 |
| 3114 | LYS382 | O | 37.276 | 50.232 | 17.115 |
| 3115 | TRP383 | N | 36.296 | 48.225 | 16.888 |
| 3116 | TRP383 | CA | 37.418 | 47.665 | 16.153 |
| 3117 | TRP383 | CB | 37.253 | 46.156 | 16.11 |
| 3118 | TRP383 | CG | 37.381 | 45.445 | 17.443 |
| 3119 | TRP383 | CD1 | 36.452 | 44.608 | 18.021 |
| 3120 | TRP383 | NE1 | 36.947 | 44.169 | 19.205 |
| 3121 | TRP383 | CE2 | 38.171 | 44.68 | 19.44 |
| 3122 | TRP383 | CZ2 | 39.06 | 44.542 | 20.495 |
| 3123 | TRP383 | CH2 | 40.283 | 45.202 | 20.458 |
| 3124 | TRP383 | CZ3 | 40.617 | 46.003 | 19.371 |
| 3125 | TRP383 | CE3 | 39.727 | 46.154 | 18.313 |
| 3126 | TRP383 | CD2 | 38.505 | 45.498 | 18.347 |
| 3127 | TRP383 | C | 37.439 | 48.227 | 14.738 |
| 3128 | TRP383 | O | 38.488 | 48.713 | 14.299 |
| 3129 | CYS384 | N | 36.261 | 48.433 | 14.169 |
| 3130 | CYS384 | CA | 36.189 | 49.08 | 12.859 |
| 3131 | CYS384 | CB | 34.873 | 48.766 | 12.157 |
| 3132 | CYS384 | SG | 35.036 | 47.735 | 10.681 |
| 3133 | CYS384 | C | 36.341 | 50.582 | 12.986 |
| 3134 | CYS384 | O | 36.976 | 51.168 | 12.109 |
| 3135 | LEU385 | N | 36.062 | 51.139 | 14.153 |
| 3136 | LEU385 | CA | 36.294 | 52.576 | 14.347 |
| 3137 | LEU385 | CB | 35.661 | 53.019 | 15.663 |
| 3138 | LEU385 | CG | 34.149 | 52.822 | 15.667 |
| 3139 | LEU385 | CD1 | 33.559 | 53.159 | 17.03 |
| 3140 | LEU385 | CD2 | 33.484 | 53.651 | 14.576 |
| 3141 | LEU385 | C | 37.792 | 52.878 | 14.379 |
| 3142 | LEU385 | O | 38.26 | 53.72 | 13.599 |
| 3143 | LEU386 | N | 38.545 | 51.992 | 15.014 |
| 3144 | LEU386 | CA | 39.999 | 52.146 | 15.073 |
| 3145 | LEU386 | CB | 40.512 | 51.246 | 16.191 |
| 3146 | LEU386 | CG | 42.024 | 51 .337 | 16.369 |
| 3147 | LEU386 | CD1 | 42.466 | 52.766 | 16.672 |
| 3148 | LEU386 | CD2 | 42.488 | 50.389 | 17.47 |
| 3149 | LEU386 | C | 40.667 | 51.762 | 13.753 |
| 3150 | LEU386 | O | 41.58 | 52.469 | 13.31 |
| 3151 | THR387 | N | 40.06 | 50.848 | 13.016 |
| 3152 | THR387 | CA | 40.623 | 50.446 | 11.724 |
| 3153 | THR387 | CB | 40.072 | 49.071 | 11.37 |
| 3154 | THR387 | OG1 | 40.515 | 48.169 | 12.373 |
| 3155 | THR387 | CG2 | 40.595 | 48.567 | 10.032 |
| 3156 | THR387 | C | 40.306 | 51.458 | 10.624 |
| 3157 | THR387 | O | 41.174 | 51.714 | 9.782 |
| 3158 | 1LEA388 | N | 39.24 | 52.222 | 10.803 |
| 3159 | 1LEA388 | CA | 38.938 | 53.324 | 9.888 |
| 3160 | 1LEA388 | CB | 37.51 | 53.803 | 10.143 |
| 3161 | 1LEA388 | CG2 | 37.242 | 55.138 | 9.464 |
| 3162 | 1LEA388 | CG1 | 36.492 | 52.778 | 9.668 |
| 3163 | 1LEA388 | OD1 | 35.087 | 53.151 | 10.126 |
| 3164 | 1LEA388 | C | 39.924 | 54.463 | 10.108 |
| 3165 | 1LEA388 | O | 40.519 | 54.94 | 9.133 |
| 3166 | 1LEA389 | N | 40.328 | 54.645 | 11.356 |
| 3167 | 1LEA389 | CA | 41.343 | 55.65 | 11.682 |
| 3168 | 1LEA389 | CB | 41.408 | 55.77 | 13.2 |
| 3169 | 1LEA389 | CG2 | 42.61 | 56.595 | 13.642 |
| 3170 | 1LEA389 | CG1 | 40.115 | 56.361 | 13.745 |
| 3171 | 1LEA389 | CD1 | 40.132 | 56.425 | 15.267 |
| 3172 | 1LEA389 | G | 42.711 | 55.26 | 11.129 |
| 3173 | 1LEA389 | O | 43.319 | 56.064 | 10.409 |
| 3174 | LEU390 | N | 43.03 | 53.977 | 11.193 |
| 3175 | LEU390 | CA | 44.323 | 53.499 | 10.693 |
| 3176 | LEU390 | CB | 44.54 | 52.079 | 11.202 |
| 3177 | LEU390 | CG | 44.637 | 52.026 | 12.721 |
| 3178 | LEU390 | CD1 | 44.618 | 50.585 | 13.216 |
| 3179 | LEU390 | CD2 | 45.87 | 52.766 | 13.229 |
| 3180 | LEU390 | C | 44.398 | 53.495 | 9.168 |
| 3181 | LEU390 | O | 45.414 | 53.933 | 8.612 |
| 3182 | LEU391 | N | 43.278 | 53.253 | 8.508 |
| 3183 | LEU391 | CA | 43.273 | 53.26 | 7.044 |
| 3184 | LEU391 | CB | 42.081 | 52.451 | 6.555 |
| 3185 | LEU391 | CG | 42.263 | 50.977 | 6.889 |
| 3186 | LEU391 | CD1 | 40.962 | 50.204 | 6.739 |
| 3187 | LEU391 | CD2 | 43.372 | 50.354 | 6.05 |
| 3188 | LEU391 | C | 43.222 | 54.675 | 6.483 |
| 3189 | LEU391 | O | 43.926 | 54.95 | 5.506 |
| 3190 | MET392 | N | 42.679 | 55.608 | 7.247 |
| 3191 | MET392 | CA | 42.705 | 57.007 | 6.816 |
| 3192 | MET392 | CB | 41.664 | 57.792 | 7.603 |
| 3193 | MET392 | CG | 40.253 | 57.411 | 7.174 |
| 3194 | MET392 | SD | 38.92 | 58.381 | 7.91 |
| 3195 | MET392 | CE | 39.254 | 58.041 | 9.65 |
| 3196 | MET392 | C | 44.084 | 57.625 | 7.019 |
| 3197 | MET392 | O | 44.577 | 58.32 | 6.119 |
| 3198 | ARG393 | N | 44.804 | 57.127 | 8.01 |
| 3199 | ARG393 | CA | 46.17 | 57.597 | 8.246 |
| 3200 | ARG393 | CB | 46.538 | 57.309 | 9.698 |
| 3201 | ARG393 | CG | 45.714 | 58.177 | 10.64 |
| 3202 | ARG393 | CD | 45.967 | 59.645 | 10.332 |
| 3203 | ARG393 | NE | 45.148 | 60.544 | 11.153 |
| 3204 | ARG393 | CZ | 45.574 | 61.761 | 11.491 |
| 3205 | ARG393 | NH1 | 46.814 | 62.13 | 11.172 |
| 3206 | ARG393 | NH2 | 44.801 | 62.569 | 12.221 |
| 3207 | ARG393 | C | 47.186 | 56.94 | 7.312 |
| 3208 | ARG393 | O | 48.235 | 57.534 | 7.038 |
| 3209 | ALA394 | N | 46.824 | 55.811 | 6.725 |
| 3210 | ALA394 | CA | 47.703 | 55.167 | 5.75 |
| 3211 | ALA394 | CB | 47.566 | 53.657 | 5.895 |
| 3212 | ALA394 | C | 47.39 | 55.575 | 4.311 |
| 3213 | ALA394 | O | 48.242 | 55.403 | 3.434 |
| 3214 | LEU395 | N | 46.216 | 56.138 | 4.075 |
| 3215 | LEU395 | CA | 45.86 | 56.586 | 2.724 |
| 3216 | LEU395 | CB | 44.368 | 56.359 | 2.512 |
| 3217 | LEU395 | CG | 44.035 | 54.885 | 2.33 |
| 3218 | LEU395 | CD1 | 42.538 | 54.64 | 2.471 |
| 3219 | LEU395 | CD2 | 44.552 | 54.38 | 0.989 |
| 3220 | LEU395 | C | 46.169 | 58.062 | 2.514 |
| 3221 | LEU395 | O | 46.704 | 58.444 | 1.467 |
| 3222 | ASP396 | N | 45.834 | 58.872 | 3.504 |
| 3223 | ASP396 | CA | 46.112 | 60.314 | 3.447 |
| 3224 | ASP396 | CB | 45.347 | 60.952 | 2.282 |
| 3225 | ASP396 | CG | 45.863 | 62.36 | 1.974 |
| 3226 | ASP396 | OD1 | 46.057 | 63.113 | 2.925 |
| 3227 | ASP396 | OD2 | 45.87 | 62.717 | 0.807 |
| 3228 | ASP396 | C | 45.689 | 60.964 | 4.761 |
| 3229 | ASP396 | O | 44.6 | 61.552 | 4.84 |
| 3230 | PRO397 | N | 46.654 | 61.102 | 5.656 |
| 3231 | PRO397 | CA | 46.372 | 61.58 | 7.015 |
| 3232 | PRO397 | CB | 47.658 | 61.374 | 7.755 |
| 3233 | PRO397 | CG | 48.738 | 60.889 | 6.802 |
| 3234 | PRO397 | CD | 48.059 | 60.73 | 5.456 |
| 3235 | PRO397 | C | 45.954 | 63.054 | 7.088 |
| 3236 | PRO397 | O | 45.132 | 63.411 | 7.942 |
| 3237 | LEU398 | N | 46.326 | 63.841 | 6.09 |
| 3238 | LEU398 | CA | 45.992 | 65.267 | 6.074 |
| 3239 | LEU398 | CB | 46.916 | 65.943 | 5.072 |
| 3240 | LEU398 | CG | 48.375 | 65.824 | 5.483 |
| 3241 | LEU398 | CD1 | 49.292 | 66.086 | 4.298 |
| 3242 | LEU398 | CD2 | 48.695 | 66.758 | 6.643 |
| 3243 | LEU398 | C | 44.556 | 65.499 | 5.632 |
| 3244 | LEU398 | O | 43.758 | 66.073 | 6.385 |
| 3245 | LEU399 | N | 44.18 | 64.823 | 4.56 |
| 3246 | LEU399 | CA | 42.849 | 65.006 | 3.97 |
| 3247 | LEU399 | CB | 42.88 | 64.392 | 2.574 |
| 3248 | LEU399 | CG | 41.55 | 64.535 | 1.845 |
| 3249 | LEU399 | CD1 | 41.2 | 66.004 | 1.632 |
| 3250 | LEU399 | CD2 | 41.588 | 63.797 | 0.512 |
| 3251 | LEU399 | C | 41.777 | 64.313 | 4.801 |
| 3252 | LEU399 | O | 40.699 | 64.872 | 5.037 |
| 3253 | TYR400 | N | 42.171 | 63.22 | 5.428 |
| 3254 | TYR400 | CA | 41.259 | 62.481 | 6.29 |
| 3255 | TYR400 | CB | 41.597 | 61.002 | 6.199 |
| 3256 | TYR400 | CG | 41 .286 | 60.365 | 4.846 |
| 3257 | TYR400 | CD1 | 42.225 | 59.545 | 4.237 |
| 3258 | TYR400 | GE1 | 41.946 | 58.959 | 3.01 |
| 3259 | TYR400 | CZ | 40.725 | 59.195 | 2.396 |
| 3260 | TYR400 | OH | 40.441 | 58.591 | 1.188 |
| 3261 | TYR400 | CE2 | 39.783 | 60.016 | 3 |
| 3262 | TYR400 | CD2 | 40.064 | 60.602 | 4.228 |
| 3263 | TYR400 | C | 41 .306 | 62.938 | 7.746 |
| 3264 | TYR400 | O | 40.54 | 62.397 | 8.551 |
| 3265 | GLU401 | N | 42.008 | 64.023 | 8.041 |
| 3266 | GLU401 | CA | 42.178 | 64.478 | 9.43 |
| 3267 | GLU401 | CB | 43.059 | 65.718 | 9.422 |
| 3268 | GLU401 | CG | 43.166 | 66.335 | 10.812 |
| 3269 | GLU401 | CD | 43.942 | 67.643 | 10.732 |
| 3270 | GLU401 | QE1 | 45.163 | 67.565 | 10.687 |
| 3271 | GLU401 | OE2 | 43.308 | 68.678 | 10.596 |
| 3272 | GLU401 | C | 40.873 | 64.854 | 10.12 |
| 3273 | GLU401 | O | 40.642 | 64.391 | 11.243 |
| 3274 | LYS402 | N | 39.938 | 65.442 | 9.39 |
| 3275 | LYS402 | CA | 38.681 | 65.842 | 10.026 |
| 3276 | LYS402 | CB | 37.965 | 66.845 | 9.13 |
| 3277 | LYS402 | CG | 36.675 | 67.33 | 9.782 |
| 3278 | LYS402 | CD | 35.949 | 68.346 | 8.911 |
| 3279 | LYS402 | CE | 34.668 | 68.828 | 9.584 |
| 3280 | LYS402 | NZ | 33.968 | 69.81 | 8.74 |
| 3281 | LYS402 | C | 37.774 | 64.641 | 10.277 |
| 3282 | LYS402 | O | 37.179 | 64.558 | 11.359 |
| 3283 | GLU403 | N | 37.954 | 63.602 | 9.475 |
| 3284 | GLU403 | CA | 37.155 | 62.388 | 9.619 |
| 3285 | GLU403 | CB | 37.187 | 61.637 | 8.296 |
| 3286 | GLU403 | CG | 36.7 | 62.503 | 7.142 |
| 3287 | GLU403 | CD | 36.891 | 61.757 | 5.825 |
| 3288 | GLU403 | OE1 | 37.009 | 60.541 | 5.874 |
| 3289 | GLU403 | OE2 | 37.062 | 62.428 | 4.817 |
| 3290 | GLU403 | C | 37.754 | 61.503 | 10.702 |
| 3291 | GLU403 | O | 37.013 | 60.984 | 11.543 |
| 3292 | THR404 | N | 39.062 | 61.615 | 10.867 |
| 3293 | THR404 | CA | 39.767 | 60.847 | 11.89 |
| 3294 | THR404 | CB | 41.256 | 60.844 | 11.567 |
| 3295 | THR404 | OG1 | 41.442 | 60.293 | 10.271 |
| 3296 | THR404 | CG2 | 42.024 | 59.987 | 12.562 |
| 3297 | THR404 | C | 39.56 | 61.459 | 13.266 |
| 3298 | THR404 | O | 39.419 | 60.722 | 14.246 |
| 3299 | LEU405 | N | 39.273 | 62.749 | 13.297 |
| 3300 | LEU405 | CA | 38.948 | 63.406 | 14.565 |
| 3301 | LEU405 | CB | 39.136 | 64.913 | 14.412 |
| 3302 | LEU405 | CG | 40.427 | 65.427 | 15.055 |
| 3303 | LEU405 | CD1 | 41.684 | 64.76 | 14.501 |
| 3304 | LEU405 | CD2 | 40.53 | 66.94 | 14.91 |
| 3305 | LEU405 | C | 37.511 | 63.103 | 14.979 |
| 3306 | LEU405 | O | 37.271 | 62.822 | 16.161 |
| 3307 | GLN406 | N | 36.652 | 62.855 | 14.001 |
| 3308 | GLN406 | CA | 35.27 | 62.498 | 14.327 |
| 3309 | GLN406 | CB | 34.35 | 62.666 | 13.119 |
| 3310 | GLN406 | OG | 34.427 | 64.043 | 12.464 |
| 3311 | GLN406 | CD | 34.233 | 65.184 | 13.462 |
| 3312 | GLN406 | OE1 | 33.285 | 65.203 | 14.255 |
| 3313 | GLN406 | NE2 | 35.166 | 66.12 | 13.412 |
| 3314 | GLN406 | C | 35.215 | 61.049 | 14.788 |
| 3315 | GLN406 | O | 34.616 | 60.769 | 15.834 |
| 3316 | TYR407 | N | 36.07 | 60.226 | 14.202 |
| 3317 | TYR407 | CA | 36.164 | 58.824 | 14.608 |
| 3318 | TYR407 | CB | 36.782 | 58.007 | 13.48 |
| 3319 | TYR407 | CG | 35.786 | 57.61 | 12.398 |
| 3320 | TYR407 | CD1 | 35.772 | 58.254 | 11.167 |
| 3321 | TYR407 | CE1 | 34.855 | 57.878 | 10.196 |
| 3322 | TYR407 | CZ | 33.956 | 56.853 | 10.458 |
| 3323 | TYR407 | OH | 33.067 | 56.448 | 9.484 |
| 3324 | TYR407 | CE2 | 33.969 | 56.206 | 11.686 |
| 3325 | TYR407 | CD2 | 34.887 | 56.584 | 12.656 |
| 3326 | TYR407 | C | 36.958 | 58.618 | 15.893 |
| 3327 | TYR407 | O | 36.66 | 57.665 | 16.617 |
| 3328 | PHE408 | N | 37.774 | 59.582 | 16.289 |
| 3329 | PHE408 | CA | 38.422 | 59.5 | 17.6 |
| 3330 | PHE408 | CB | 39.641 | 60.411 | 17.638 |
| 3331 | PHE408 | CG | 40.956 | 59.677 | 17.414 |
| 3332 | PHE408 | CD1 | 41.786 | 60.022 | 16.355 |
| 3333 | PHE408 | CE1 | 42.983 | 59.345 | 16.164 |
| 3334 | PHE408 | CZ | 43.351 | 58.325 | 17.032 |
| 3335 | PHE408 | CE2 | 42.523 | 57.982 | 18.092 |
| 3336 | PHE408 | CD2 | 41.326 | 58.659 | 18.283 |
| 3337 | PHE408 | C | 37.463 | 59.891 | 18.712 |
| 3338 | PHE408 | O | 37.428 | 59.208 | 19.742 |
| 3339 | GLN409 | N | 36.522 | 60.768 | 18.401 |
| 3340 | GLN409 | CA | 35.486 | 61.115 | 19.377 |
| 3341 | GLN409 | CB | 34.801 | 62.395 | 18.916 |
| 3342 | GLN409 | CG | 35.771 | 63.57 | 18.884 |
| 3343 | GLN409 | CD | 35.105 | 64.765 | 18.212 |
| 3344 | GLN409 | OE1 | 35.266 | 65.915 | 18.638 |
| 3345 | GLN409 | NE2 | 34.379 | 64.475 | 17.147 |
| 3346 | GLN409 | C | 34.452 | 59.998 | 19.489 |
| 3347 | GLN409 | O | 34.075 | 59.62 | 20.606 |
| 3348 | THR410 | N | 34.228 | 59.305 | 18.385 |
| 3349 | THR410 | CA | 33.288 | 58.181 | 18.384 |
| 3350 | THR410 | CB | 32.95 | 57.837 | 16.936 |
| 3351 | THR410 | OG1 | 32.383 | 58.99 | 16.327 |
| 3352 | THR410 | CG2 | 31.934 | 56.705 | 16.847 |
| 3353 | THR410 | C | 33.891 | 56.958 | 19.067 |
| 3354 | THR410 | O | 33.246 | 56.373 | 19.944 |
| 3355 | LEU411 | N | 35.189 | 56.778 | 18.897 |
| 3356 | LEU411 | CA | 35.89 | 55.648 | 19.506 |
| 3357 | LEU411 | CB | 37.218 | 55.515 | 18.769 |
| 3358 | LEU411 | CG | 38.034 | 54.309 | 19.206 |
| 3359 | LEU411 | CD1 | 37.219 | 53.03 | 19.101 |
| 3360 | LEU411 | CD2 | 39.3 | 54.202 | 18.368 |
| 3361 | LEU411 | C | 36.123 | 55.867 | 21 |
| 3362 | LEU411 | O | 35.942 | 54.925 | 21.781 |
| 3363 | LYS412 | N | 36.212 | 57.124 | 21.404 |
| 3364 | LYS412 | CA | 36.354 | 57.46 | 22.822 |
| 3365 | LYS412 | CB | 36.878 | 58.893 | 22.886 |
| 3366 | LYS412 | CG | 37.07 | 59.396 | 24.31 |
| 3367 | LYS412 | CD | 37.628 | 60.815 | 24.317 |
| 3368 | LYS412 | CE | 37.835 | 61.328 | 25.739 |
| 3369 | LYS412 | NZ | 38.358 | 62.705 | 25.736 |
| 3370 | LYS412 | C | 35.018 | 57.353 | 23.558 |
| 3371 | LYS412 | O | 34.98 | 56.877 | 24.7 |
| 3372 | ALA413 | N | 33.93 | 57.558 | 22.832 |
| 3373 | ALA413 | CA | 32.595 | 57.425 | 23.426 |
| 3374 | ALA413 | CB | 31.632 | 58.321 | 22.655 |
| 3375 | ALA413 | C | 32.079 | 55.986 | 23.408 |
| 3376 | ALA413 | O | 31.189 | 55.641 | 24.194 |
| 3377 | VAL414 | N | 32.66 | 55.149 | 22.563 |
| 3378 | VAL414 | CA | 32.315 | 53.725 | 22.568 |
| 3379 | VAL414 | CB | 32.429 | 53.189 | 21.14 |
| 3380 | VAL414 | CG1 | 32.297 | 51.672 | 21.082 |
| 3381 | VAL414 | CG2 | 31.391 | 53.838 | 20.232 |
| 3382 | VAL414 | C | 33.236 | 52.96 | 23.515 |
| 3383 | VAL414 | O | 32.857 | 51 .926 | 24.081 |
| 3384 | ASP415 | N | 34.409 | 53.516 | 23.759 |
| 3385 | ASP415 | CA | 35.307 | 52.919 | 24.744 |
| 3386 | ASP415 | CB | 36.366 | 52.112 | 23.995 |
| 3387 | ASP415 | CG | 37.098 | 51.161 | 24.94 |
| 3388 | ASP415 | OD1 | 37.234 | 51.507 | 26.11 |
| 3389 | ASP415 | OD2 | 37.609 | 50.164 | 24.456 |
| 3390 | ASP415 | C | 35.958 | 53.997 | 25.612 |
| 3391 | ASP415 | O | 37.147 | 54.301 | 25.44 |
| 3392 | PRO416 | N | 35.279 | 54.33 | 26.701 |
| 3393 | PRO416 | CA | 35.788 | 55.333 | 27.645 |
| 3394 | PRO416 | CB | 34.602 | 55.689 | 28.488 |
| 3395 | PRO416 | CG | 33.483 | 54.69 | 28.227 |
| 3396 | PRO416 | CD | 33.984 | 53.779 | 27.119 |
| 3397 | PRO416 | C | 36.94 | 54.837 | 28.533 |
| 3398 | PRO416 | O | 37.689 | 55.663 | 29.066 |
| 3399 | MET417 | N | 37.208 | 53.539 | 28.531 |
| 3400 | MET417 | CA | 38.308 | 52.997 | 29.331 |
| 3401 | MET417 | CB | 38.027 | 51.516 | 29.546 |
| 3402 | MET417 | CG | 36.645 | 51.304 | 30.152 |
| 3403 | MET417 | 5D | 36.105 | 49.583 | 30.254 |
| 3404 | MET417 | CE | 36.11 | 49.189 | 28.489 |
| 3405 | MET417 | C | 39.618 | 53.157 | 28.57 |
| 3406 | MET417 | O | 40.664 | 53.471 | 29.15 |
| 3407 | ARG418 | N | 39.48 | 53.181 | 27.255 |
| 3408 | ARG418 | CA | 40.607 | 53.398 | 26.353 |
| 3409 | ARG418 | CB | 40.369 | 52.58 | 25.09 |
| 3410 | ARG418 | CG | 41.644 | 51.903 | 24.606 |
| 3411 | ARG418 | CD | 42.063 | 50.797 | 25.569 |
| 3412 | ARG418 | NE | 41.007 | 49.775 | 25.666 |
| 3413 | ARG418 | CZ | 40.523 | 49.319 | 26.824 |
| 3414 | ARG418 | NH1 | 41.04 | 49.747 | 27.978 |
| 3415 | ARG418 | NH2 | 39.552 | 48.403 | 26.827 |
| 3416 | ARG418 | C | 40.725 | 54.867 | 25.962 |
| 3417 | ARG418 | O | 41.636 | 55.216 | 25.202 |
| 3418 | ALA419 | N | 39.935 | 55.729 | 26.587 |
| 3419 | ALA419 | CA | 39.842 | 57.132 | 26.168 |
| 3420 | ALA419 | CB | 38.795 | 57.822 | 27.032 |
| 3421 | ALA419 | C | 41.154 | 57.894 | 26.284 |
| 3422 | ALA419 | O | 41.573 | 58.501 | 25.291 |
| 3423 | THR420 | N | 41.935 | 57.597 | 27.312 |
| 3424 | THR420 | CA | 43.225 | 58.282 | 27.478 |
| 3425 | THR420 | CB | 43.737 | 58.049 | 28.897 |
| 3426 | THR420 | OG1 | 43.951 | 56.657 | 29.09 |
| 3427 | THR420 | CG2 | 42.729 | 58.522 | 29.938 |
| 3428 | THR420 | C | 44.268 | 57.808 | 26.462 |
| 3429 | THR420 | O | 44.94 | 58.659 | 25.862 |
| 3430 | TYR421 | N | 44.133 | 56.571 | 26.009 |
| 3431 | TYR421 | CA | 45.043 | 56.039 | 24.996 |
| 3432 | TYR421 | CB | 44.96 | 54.516 | 25 |
| 3433 | TYR421 | CG | 45.623 | 53.866 | 23.788 |
| 3434 | TYR421 | CD1 | 47.005 | 53.9 | 23.646 |
| 3435 | TYR421 | CE1 | 47.603 | 53.321 | 22.534 |
| 3436 | TYR421 | CZ | 46.815 | 52.713 | 21.565 |
| 3437 | TYR421 | OH | 47.405 | 52.167 | 20.447 |
| 3438 | TYR421 | CE2 | 45.435 | 52.675 | 21.705 |
| 3439 | TYR421 | CD2 | 44.839 | 53.253 | 22.818 |
| 3440 | TYR421 | C | 44.669 | 56.56 | 23.616 |
| 3441 | TYR421 | O | 45.56 | 56.964 | 22.86 |
| 3442 | LEU422 | N | 43.39 | 56.841 | 23.428 |
| 3443 | LEU422 | CA | 42.922 | 57.4 | 22.158 |
| 3444 | LEU422 | CB | 41.417 | 57.185 | 22.071 |
| 3445 | LEU422 | CG | 41.083 | 55.702 | 22.17 |
| 3446 | LEU422 | CD1 | 39.586 | 55.478 | 22.334 |
| 3447 | LEU422 | OD2 | 41.638 | 54.924 | 20.983 |
| 3448 | LEU422 | C | 43.241 | 58.887 | 22.067 |
| 3449 | LEU422 | O | 43.668 | 59.347 | 21.003 |
| 3450 | ASP423 | N | 43.354 | 59.529 | 23.219 |
| 3451 | ASP423 | CA | 43.766 | 60.934 | 23.269 |
| 3452 | ASP423 | CB | 43.44 | 61.492 | 24.652 |
| 3453 | ASP423 | CG | 41.951 | 61.379 | 24.969 |
| 3454 | ASP423 | OD1 | 41.156 | 61.541 | 24.053 |
| 3455 | ASP423 | OD2 | 41.633 | 61.25 | 26.146 |
| 3456 | ASP423 | C | 45.27 | 61.065 | 23.035 |
| 3457 | ASP423 | O | 45.71 | 61.971 | 22.318 |
| 3458 | ASP424 | N | 46.012 | 60.04 | 23.423 |
| 3459 | ASP424 | CA | 47.46 | 60.028 | 23.198 |
| 3460 | ASP424 | CB | 48.091 | 58.993 | 24.128 |
| 3461 | ASP424 | CG | 47.868 | 59.352 | 25.596 |
| 3462 | ASP424 | OD1 | 47.843 | 60.539 | 25.895 |
| 3463 | ASP424 | OD2 | 47.81 | 58.432 | 26.403 |
| 3464 | ASP424 | C | 47.798 | 59.659 | 21.755 |
| 3465 | ASP424 | O | 48.654 | 60.307 | 21.138 |
| 3466 | LEU425 | N | 46.965 | 58.822 | 21.158 |
| 3467 | LEU425 | CA | 47.178 | 58.392 | 19.775 |
| 3468 | LEU425 | CB | 46.375 | 57.111 | 19.573 |
| 3469 | LEU425 | OG | 46.664 | 56.449 | 18.231 |
| 3470 | LEU425 | CD1 | 48.144 | 56.104 | 18.104 |
| 3471 | LEU425 | CD2 | 45.808 | 55.2 | 18.05 |
| 3472 | LEU425 | C | 46.719 | 59.465 | 18.79 |
| 3473 | LEU425 | O | 47.377 | 59.687 | 17.765 |
| 3474 | ARG426 | N | 45.777 | 60.283 | 19.228 |
| 3475 | ARG426 | CA | 45.335 | 61.422 | 18.426 |
| 3476 | ARG426 | CB | 43.961 | 61.834 | 18.932 |
| 3477 | ARG426 | OG | 43.405 | 63.039 | 18.189 |
| 3478 | ARG426 | CD | 42.048 | 63.42 | 18.768 |
| 3479 | ARG426 | NE | 42.121 | 63.464 | 20.239 |
| 3480 | ARG426 | CZ | 42.439 | 64.553 | 20.942 |
| 3481 | ARG426 | NH1 | 42.659 | 65.713 | 20.32 |
| 3482 | ARG426 | NH2 | 42.501 | 64.488 | 22.274 |
| 3483 | ARG426 | C | 46.313 | 62.587 | 18.543 |
| 3484 | ARG426 | O | 46.562 | 63.268 | 17.541 |
| 3485 | SER427 | N | 47.051 | 62.632 | 19.642 |
| 3486 | SER427 | CA | 48.124 | 63.623 | 19.782 |
| 3487 | SER427 | CB | 48.648 | 63.604 | 21.211 |
| 3488 | SER427 | OG | 47.599 | 63.964 | 22.09 |
| 3489 | SER427 | C | 49.28 | 63.28 | 18.855 |
| 3490 | SER427 | O | 49.718 | 64.13 | 18.068 |
| 3491 | LYS428 | N | 49.555 | 61.989 | 18.763 |
| 3492 | LYS428 | CA | 50.604 | 61.484 | 17.879 |
| 3493 | LYS428 | CB | 50.703 | 59.984 | 18.118 |
| 3494 | LYS428 | CG | 51.857 | 59.354 | 17.353 |
| 3495 | LYS428 | CD | 51.883 | 57.848 | 17.575 |
| 3496 | LYS428 | CE | 51.959 | 57.519 | 19.061 |
| 3497 | LYS428 | NZ | 51.938 | 56.066 | 19.282 |
| 3498 | LYS428 | C | 50.271 | 61.741 | 16.414 |
| 3499 | LYS428 | O | 51.036 | 62.436 | 15.731 |
| 3500 | PHE429 | N | 49.037 | 61.453 | 16.033 |
| 3501 | PHE429 | CA | 48.621 | 61.629 | 14.639 |
| 3502 | PHE429 | CB | 47.283 | 60.925 | 14.452 |
| 3503 | PHE429 | CG | 47.345 | 59.403 | 14.376 |
| 3504 | PHE429 | CD1 | 46.236 | 58.65 | 14.733 |
| 3505 | PHE429 | CE1 | 46.282 | 57.265 | 14.658 |
| 3506 | PHE429 | CZ | 47.437 | 56.63 | 14.22 |
| 3507 | PHE429 | CE2 | 48.544 | 57.384 | 13.851 |
| 3508 | PHE429 | CD2 | 48.496 | 58.77 | 13.924 |
| 3509 | PHE429 | C | 48.473 | 63.093 | 14.222 |
| 3510 | PHE429 | O | 48.938 | 63.462 | 13.135 |
| 3511 | LEU430 | N | 48.099 | 63.954 | 15.152 |
| 3512 | LEU430 | CA | 47.916 | 65.364 | 14.809 |
| 3513 | LEU430 | CB | 46.953 | 65.956 | 15.829 |
| 3514 | LEU430 | CG | 46.363 | 67.28 | 15.366 |
| 3515 | LEU430 | CD1 | 45.871 | 67.181 | 13.927 |
| 3516 | LEU430 | CD2 | 45.227 | 67.696 | 16.294 |
| 3517 | LEU430 | C | 49.251 | 66.112 | 14.801 |
| 3518 | LEU430 | O | 49.446 | 67.011 | 13.97 |
| 3519 | LEU431 | N | 50.231 | 65.557 | 15.496 |
| 3520 | LEU431 | CA | 51.586 | 66.104 | 15.444 |
| 3521 | LEU431 | CB | 52.335 | 65.63 | 16.69 |
| 3522 | LEU431 | CG | 53.656 | 66.362 | 16.92 |
| 3523 | LEU431 | CD1 | 53.952 | 66.49 | 18.409 |
| 3524 | LEU431 | CD2 | 54.832 | 65.726 | 16.183 |
| 3525 | LEU431 | C | 52.276 | 65.633 | 14.166 |
| 3526 | LEU431 | O | 52.953 | 66.436 | 13.511 |
| 3527 | GLU432 | N | 51.872 | 64.47 | 13.681 |
| 3528 | GLU432 | CA | 52.381 | 63.959 | 12.403 |
| 3529 | GLU432 | CB | 51.895 | 62.529 | 12.22 |
| 3530 | GLU432 | CG | 52.527 | 61.594 | 13.238 |
| 3531 | GLU432 | CD | 51.87 | 60.222 | 13.152 |
| 3532 | GLU432 | OE1 | 51.442 | 59.865 | 12.064 |
| 3533 | GLU432 | OE2 | 51.697 | 59.61 | 14.197 |
| 3534 | GLU432 | C | 51.881 | 64.799 | 11.235 |
| 3535 | GLU432 | O | 52.702 | 65.259 | 10.431 |
| 3536 | ASN433 | N | 50.634 | 65.235 | 11.317 |
| 3537 | ASN433 | CA | 50.093 | 66.124 | 10.287 |
| 3538 | ASN433 | CB | 48.591 | 66.227 | 10.458 |
| 3539 | ASN433 | CG | 47.889 | 65.099 | 9.726 |
| 3540 | ASN433 | OD1 | 48.513 | 64.222 | 9.115 |
| 3541 | ASN433 | ND2 | 46.58 | 65.231 | 9.688 |
| 3542 | ASN433 | C | 50.668 | 67.53 | 10.347 |
| 3543 | ASN433 | O | 50.95 | 68.101 | 9.287 |
| 3544 | SER434 | N | 51.084 | 67.971 | 11.522 |
| 3545 | SER434 | CA | 51.693 | 69.298 | 11.625 |
| 3546 | SER434 | CB | 51.649 | 69.751 | 13.076 |
| 3547 | SER434 | OG | 50.284 | 69.827 | 13.464 |
| 3548 | SER434 | C | 53.135 | 69.289 | 11.127 |
| 3549 | SER434 | O | 53.557 | 70.266 | 10.498 |
| 3550 | VAL435 | N | 53.779 | 68.132 | 11.169 |
| 3551 | VAL435 | CA | 55.123 | 68.004 | 10.597 |
| 3552 | VAL435 | CB | 55.816 | 66.803 | 11.232 |
| 3553 | VAL435 | CG1 | 57.185 | 66.563 | 10.611 |
| 3554 | VAL435 | CG2 | 55.95 | 66.986 | 12.738 |
| 3555 | VAL435 | C | 55.056 | 67.826 | 9.08 |
| 3556 | VAL435 | O | 55.892 | 68.388 | 8.359 |
| 3557 | LEU436 | N | 53.947 | 67.286 | 8.6 |
| 3558 | LEU436 | CA | 53.722 | 67.207 | 7.153 |
| 3559 | LEU436 | CB | 52.506 | 66.328 | 6.884 |
| 3560 | LEU436 | CG | 52.774 | 64.867 | 7.215 |
| 3561 | LEU436 | CD1 | 51.489 | 64.048 | 7.172 |
| 3562 | LEU436 | CD2 | 53.821 | 64.28 | 6.277 |
| 3563 | LEU436 | C | 53.473 | 68.592 | 6.571 |
| 3564 | LEU436 | O | 54.192 | 68.996 | 5.649 |
| 3565 | LYS437 | N | 52.704 | 69.4 | 7.286 |
| 3566 | LYS437 | CA | 52.418 | 70.771 | 6.84 |
| 3567 | LYS437 | CB | 51.28 | 71.316 | 7.695 |
| 3568 | LYS437 | CG | 50.023 | 70.468 | 7.552 |
| 3569 | LYS437 | CD | 48.97 | 70.867 | 8.58 |
| 3570 | LYS437 | CE | 47.756 | 69.948 | 8.516 |
| 3571 | LYS437 | NZ | 46.775 | 70.306 | 9.552 |
| 3572 | LYS437 | C | 53.63 | 71 .688 | 6.985 |
| 3573 | LYS437 | O | 53.894 | 72.488 | 6.079 |
| 3574 | MET438 | N | 54.495 | 71.372 | 7.937 |
| 3575 | MET438 | CA | 55.739 | 72.122 | 8.121 |
| 3576 | MET438 | CB | 56.323 | 71.717 | 9.471 |
| 3577 | MET438 | CG | 57.636 | 72.428 | 9.765 |
| 3578 | MET438 | SD | 58.438 | 71.992 | 11.324 |
| 3579 | MET438 | CE | 58.669 | 70.223 | 11.034 |
| 3580 | MET438 | C | 56.751 | 71.823 | 7.014 |
| 3581 | MET438 | O | 57.447 | 72.741 | 6.56 |
| 3582 | GLU439 | N | 56.641 | 70.648 | 6.414 |
| 3583 | GLU439 | CA | 57.507 | 70.292 | 5.29 |
| 3584 | GLU439 | CR | 57.588 | 68.776 | 5.211 |
| 3585 | GLU439 | CG | 58.283 | 68.224 | 6.441 |
| 3586 | GLU439 | CD | 58.201 | 66.706 | 6.461 |
| 3587 | GLU439 | QE1 | 57.871 | 66.112 | 5.442 |
| 3588 | GLU439 | OE2 | 58.624 | 66.14 | 7.457 |
| 3589 | GLU439 | C | 56.973 | 70.823 | 3.968 |
| 3590 | GLU439 | O | 57.77 | 71.284 | 3.144 |
| 3591 | TYR440 | N | 55.664 | 71.001 | 3.877 |
| 3592 | TYR440 | CA | 55.071 | 71.506 | 2.63 |
| 3593 | TYR440 | CB | 53.63 | 71.016 | 2.517 |
| 3594 | TYR440 | OG | 53.47 | 69.497 | 2.55 |
| 3595 | TYR440 | CO1 | 54.404 | 68.674 | 1.931 |
| 3596 | TYR440 | GE1 | 54.261 | 67.294 | 1.989 |
| 3597 | TYR440 | CZ | 53.175 | 66.742 | 2.654 |
| 3598 | TYR440 | OH | 53.096 | 65.376 | 2.822 |
| 3599 | TYR440 | CE2 | 52.222 | 67.562 | 3.241 |
| 3600 | TYR440 | CD2 | 52.366 | 68.941 | 3.183 |
| 3601 | TYR440 | C | 55.106 | 73.032 | 2.575 |
| 3602 | TYR440 | O | 54.901 | 73.629 | 1.513 |
| 3603 | ALA441 | N | 55.358 | 73.646 | 3.719 |
| 3604 | ALA441 | CA | 55.625 | 75.083 | 3.769 |
| 3605 | ALA441 | CB | 54.872 | 75.677 | 4.953 |
| 3606 | ALA441 | C | 57.119 | 75.373 | 3.908 |
| 3607 | ALA441 | O | 57.524 | 76.539 | 3.806 |
| 3608 | GLU442 | N | 57.919 | 74.313 | 3.955 |
| 3609 | GLU442 | CA | 59.365 | 74.349 | 4.261 |
| 3610 | GLU442 | CR | 60.23 | 74.51 | 2.996 |
| 3611 | GLU442 | CG | 59.991 | 75.764 | 2.148 |
| 3612 | GLU442 | CD | 59.111 | 75.467 | 0.934 |
| 3613 | GLU442 | OE1 | 59.262 | 76.169 | −0.057 |
| 3614 | GLU442 | OE2 | 58.406 | 74.467 | 0.97 |
| 3615 | GLU442 | C | 59.715 | 75.389 | 5.328 |
| 3616 | GLU442 | O | 60.44 | 76.361 | 5.079 |
| 3617 | VAL443 | N | 59.199 | 75.161 | 6.524 |
| 3618 | VAL443 | CA | 59.423 | 76.095 | 7.631 |
| 3619 | VAL443 | CCB | 58.098 | 76.742 | 8.02 |
| 3620 | VAL443 | CG1 | 57.662 | 77.79 | 7.003 |
| 3621 | VAL443 | CG2 | 57.007 | 75.702 | 8.238 |
| 3622 | VAL443 | C | 60.051 | 75.414 | 8.842 |
| 3623 | VAL443 | O | 60.146 | 74.186 | 8.92 |
| 3624 | ARG444 | N | 60.565 | 76.243 | 9.737 |
| 3625 | ARG444 | CA | 61.135 | 75.758 | 11.001 |
| 3626 | ARG444 | CB | 62.499 | 76.408 | 11.217 |
| 3627 | ARG444 | CG | 63.371 | 76.224 | 9.977 |
| 3628 | ARG444 | CD | 64.806 | 76.694 | 10.189 |
| 3629 | ARG444 | NE | 65.557 | 75.752 | 11.033 |
| 3630 | ARG444 | CZ | 66.74 | 76.044 | 11.579 |
| 3631 | ARG444 | NH1 | 67.263 | 77.262 | 11.422 |
| 3632 | ARG444 | NH2 | 67.38 | 75.133 | 12.313 |
| 3633 | ARG444 | C | 60.197 | 76.043 | 12.177 |
| 3634 | ARG444 | O | 60.617 | 76.031 | 13.344 |
| 3635 | VAL445 | N | 58.987 | 76.468 | 11.85 |
| 3636 | VAL445 | CA | 57.945 | 76.673 | 12.86 |
| 3637 | VAL445 | CB | 57.195 | 77.971 | 12.557 |
| 3638 | VAL445 | CG1 | 58.147 | 79.159 | 12.543 |
| 3639 | VAL445 | CG2 | 56.444 | 77.908 | 11.232 |
| 3640 | VAL445 | C | 56.981 | 75.485 | 12.87 |
| 3641 | VAL445 | O | 56.641 | 74.929 | 11.819 |
| 3642 | LEU446 | N | 56.597 | 75.069 | 14.062 |
| 3643 | LEU446 | CA | 55.655 | 73.954 | 14.198 |
| 3644 | LEU446 | CB | 56.353 | 72.786 | 14.884 |
| 3645 | LEU446 | CG | 55.487 | 71.531 | 14.853 |
| 3646 | LEU446 | CD1 | 55.189 | 71.125 | 13.416 |
| 3647 | LEU446 | CD2 | 56.153 | 70.383 | 15.598 |
| 3648 | LEU446 | C | 54.429 | 74.382 | 14.999 |
| 3649 | LEU446 | O | 54.512 | 74.717 | 16.191 |
| 3650 | HIS447 | N | 53.294 | 74.375 | 14.323 |
| 3651 | HIS447 | CA | 52.042 | 74.804 | 14.951 |
| 3652 | HIS447 | CB | 51.262 | 75.64 | 13.948 |
| 3653 | HIS447 | CG | 52.022 | 76.876 | 13.511 |
| 3654 | HIS447 | ND1 | 52.232 | 77.286 | 12.246 |
| 3655 | HIS447 | CE1 | 52.958 | 78.422 | 12.259 |
| 3656 | HIS447 | NE2 | 53.213 | 78.73 | 13.551 |
| 3657 | HIS447 | CD2 | 52.643 | 77.787 | 14.334 |
| 3658 | HIS447 | C | 51.212 | 73.619 | 15.429 |
| 3659 | HIS447 | O | 50.802 | 72.749 | 14.652 |
| 3660 | LEU448 | N | 51.055 | 73.576 | 16.74 |
| 3661 | LEU448 | CA | 50.26 | 72.565 | 17.434 |
| 3662 | LEU448 | CB | 51.209 | 71.637 | 18.18 |
| 3663 | LEU448 | CG | 51.959 | 70.717 | 17.226 |
| 3664 | LEU448 | CD1 | 53.12 | 70.028 | 17.927 |
| 3665 | LEU448 | CD2 | 51.008 | 69.698 | 16.611 |
| 3666 | LEU448 | C | 49.315 | 73.228 | 18.434 |
| 3667 | LEU448 | O | 48.931 | 72.614 | 19.438 |
| 3668 | ALA449 | N | 49.046 | 74.503 | 18.212 |
| 3669 | ALA449 | CA | 48.176 | 75.268 | 19.109 |
| 3670 | ALA449 | CB | 48.369 | 76.752 | 18.841 |
| 3671 | ALA449 | C | 46.711 | 74.916 | 18.907 |
| 3672 | ALA449 | O | 46.262 | 74.753 | 17.765 |
| 3673 | HIS450 | N | 45.994 | 74.831 | 20.018 |
| 3674 | HIS450 | CA | 44.56 | 74.502 | 20.034 |
| 3675 | HIS450 | CB | 43.757 | 75.577 | 19.301 |
| 3676 | HIS450 | CG | 43.689 | 76.941 | 19.957 |
| 3677 | HIS450 | ND1 | 42.738 | 77.361 | 20.813 |
| 3678 | HIS450 | CE1 | 42.996 | 78.633 | 21.178 |
| 3679 | HIS450 | NE2 | 44.122 | 79.022 | 20.538 |
| 3680 | HIS450 | CD2 | 44.558 | 77.991 | 19.778 |
| 3681 | HIS450 | C | 44.285 | 73.169 | 19.354 |
| 3682 | HIS450 | O | 43.405 | 73.087 | 18.489 |
| 3683 | LYS451 | N | 45.039 | 72.144 | 19.713 |
| 3684 | LYS451 | CA | 44.853 | 70.848 | 19.054 |
| 3685 | LYS451 | CB | 46.182 | 70.387 | 18.473 |
| 3686 | LYS451 | CG | 46.684 | 71.316 | 17.376 |
| 3687 | LYS451 | CD | 45.718 | 71.402 | 16.201 |
| 3688 | LYS451 | CE | 46.264 | 72.329 | 15.124 |
| 3689 | LYS451 | NZ | 47.573 | 71.854 | 14.649 |
| 3690 | LYS451 | C | 44.329 | 69.805 | 20.028 |
| 3691 | LYS451 | O | 44.011 | 68.675 | 19.636 |
| 3692 | ASP452 | N | 44.315 | 70.19 | 21.295 |
| 3693 | ASP452 | CA | 43.867 | 69.351 | 22.414 |
| 3694 | ASP452 | CB | 42.432 | 68.895 | 22.157 |
| 3695 | ASP452 | CG | 41.763 | 68.503 | 23.465 |
| 3696 | ASP452 | OD1 | 42 | 69.203 | 24.438 |
| 3697 | ASP452 | OD2 | 40.966 | 67.576 | 23.449 |
| 3698 | ASP452 | C | 44.816 | 68.162 | 22.596 |
| 3699 | ASP452 | O | 44.406 | 67.053 | 22.954 |
| 3700 | LEU453 | N | 46.099 | 68.437 | 22.426 |
| 3701 | LEU453 | CA | 47.126 | 67.396 | 22.532 |
| 3702 | LEU453 | CB | 48.434 | 67.897 | 21.934 |
| 3703 | LEU453 | CG | 48.301 | 68.306 | 20.475 |
| 3704 | LEU453 | CO1 | 49.619 | 68.877 | 19.971 |
| 3705 | LEU453 | CD2 | 47.857 | 67.142 | 19.597 |
| 3706 | LEU453 | C | 47.381 | 67.045 | 23.985 |
| 3707 | LEU453 | O | 47.506 | 67.937 | 24.831 |
| 3708 | THR454 | N | 47.537 | 65.761 | 24.242 |
| 3709 | THR454 | CA | 47.783 | 65.28 | 25.602 |
| 3710 | THR454 | CR | 46.856 | 64.099 | 25.882 |
| 3711 | THR454 | OG1 | 47.061 | 63.099 | 24.89 |
| 3712 | THR454 | CG2 | 45.396 | 64.522 | 25.816 |
| 3713 | THR454 | C | 49.241 | 64.868 | 25.792 |
| 3714 | THR454 | O | 49.812 | 65.054 | 26.874 |
| 3715 | VAL455 | N | 49.867 | 64.441 | 24.708 |
| 3716 | VAL455 | CA | 51.276 | 64.027 | 24.774 |
| 3718 | VAL455 | CG1 | 50.636 | 61.756 | 23.861 |
| 3719 | VAL455 | CG2 | 52.721 | 61.979 | 25.228 |
| 3720 | VAL455 | C | 52.035 | 64.425 | 23.504 |
| 3721 | VAL455 | O | 51.5 | 64.334 | 22.392 |
| 3722 | LEU456 | N | 53.229 | 64.965 | 23.69 |
| 3723 | LEU456 | CA | 54.09 | 65.311 | 22.553 |
| 3724 | LEU456 | CB | 55.107 | 66.35 | 23.003 |
| 3725 | LEU456 | OG | 54.441 | 67.656 | 23.405 |
| 3726 | LEU456 | OD1 | 55.47 | 68.63 | 23.964 |
| 3727 | LEU456 | CD2 | 53.697 | 68.269 | 22.223 |
| 3728 | LEU456 | C | 54.835 | 64.086 | 22.03 |
| 3729 | LEU456 | O | 55.579 | 63.429 | 22.766 |
| 3730 | CYS457 | N | 54.634 | 63.798 | 20.757 |
| 3731 | CY3457 | CA | 55.31 | 62.661 | 20.125 |
| 3732 | CYS457 | CB | 54.251 | 61.735 | 19.546 |
| 3733 | CYS457 | SG | 53.099 | 61.052 | 20.762 |
| 3734 | CYS457 | C | 56.279 | 63.12 | 19.039 |
| 3735 | CYS457 | O | 56.304 | 64.301 | 18.679 |
| 3736 | HIS458 | N | 57.136 | 62.196 | 18.624 |
| 3737 | HIS458 | CA | 58.131 | 62.408 | 17.551 |
| 3738 | HIS458 | CB | 57.409 | 62.56 | 16.212 |
| 3739 | HIS458 | CG | 56.641 | 61.337 | 15.753 |
| 3740 | HIS458 | ND1 | 57.146 | 60.28 | 15.089 |
| 3741 | HIS458 | CE1 | 56.162 | 59.389 | 14.852 |
| 3742 | HIS458 | NE2 | 55.018 | 59.896 | 15.367 |
| 3743 | HIS458 | CD2 | 55.296 | 61.098 | 15.921 |
| 3744 | HIS458 | C | 59.014 | 63.636 | 17.757 |
| 3745 | HIS458 | O | 59.415 | 64.275 | 16.775 |
| 3746 | LEU459 | N | 59.535 | 63.79 | 18.964 |
| 3747 | LEU459 | CA | 60.282 | 65.009 | 19.3 |
| 3748 | LEU459 | CB | 60.256 | 65.183 | 20.812 |
| 3749 | LEU459 | CG | 58.834 | 65.391 | 21 .323 |
| 3750 | LEU459 | CD1 | 58.787 | 65.358 | 22.845 |
| 3751 | LEU459 | CD2 | 58.241 | 66.692 | 20.793 |
| 3752 | LEU459 | C | 61.721 | 64.962 | 18.796 |
| 3753 | LEU459 | O | 62.298 | 66.004 | 18.472 |
| 3754 | GLU460 | N | 62.158 | 63.766 | 18.438 |
| 3755 | GLU460 | CA | 63.492 | 63.579 | 17.863 |
| 3756 | GLU460 | CB | 63.997 | 62.158 | 18.141 |
| 3757 | GLU460 | CG | 63.548 | 61.058 | 17.168 |
| 3758 | GLU460 | CD | 62.096 | 60.619 | 17.347 |
| 3759 | GLU460 | OE1 | 61.52 | 60.948 | 18.38 |
| 3760 | GLU460 | OE2 | 61.509 | 60.243 | 16.339 |
| 3761 | GLU460 | C | 63.539 | 63.871 | 16.36 |
| 3762 | GLU460 | O | 64.628 | 63.868 | 15.779 |
| 3763 | GLN461 | N | 62.396 | 64.153 | 15.749 |
| 3764 | GLN461 | CA | 62.395 | 64.568 | 14.346 |
| 3765 | GLN461 | CB | 61.121 | 64.051 | 13.677 |
| 3766 | GLN461 | CG | 60.967 | 62.534 | 13.772 |
| 3767 | GLN461 | CD | 62.079 | 61.821 | 13.005 |
| 3768 | GLN461 | OE1 | 62.41 | 62.189 | 11.872 |
| 3769 | GLN461 | NE2 | 62.576 | 60.75 | 13.598 |
| 3770 | GLN461 | C | 62.401 | 66.092 | 14.282 |
| 3771 | GLN461 | O | 62.903 | 66.686 | 13.322 |
| 3772 | LEU462 | N | 62.104 | 66.695 | 15.421 |
| 3773 | LEU462 | CA | 61.88 | 68.138 | 15.498 |
| 3774 | LEU462 | CB | 60.734 | 68.368 | 16.474 |
| 3775 | LEU462 | CG | 59.476 | 67.62 | 16.044 |
| 3776 | LEU462 | CD1 | 58.393 | 67.709 | 17.112 |
| 3777 | LEU462 | CD2 | 58.958 | 68.127 | 14.701 |
| 3778 | LEU462 | C | 63.11 | 68.937 | 15.934 |
| 3779 | LEU462 | O | 62.951 | 70.078 | 16.38 |
| 3780 | LEU463 | N | 64.299 | 68.449 | 15.607 |
| 3781 | LEU463 | CA | 65.56 | 69.094 | 16.02 |
| 3782 | LEU463 | CB | 66.699 | 68.213 | 15.499 |
| 3783 | LEU463 | CG | 68.084 | 68.859 | 15.598 |
| 3784 | LEU463 | CD1 | 68.502 | 69.107 | 17.041 |
| 3785 | LEU463 | CD2 | 69.135 | 68.004 | 14.9 |
| 3786 | LEU463 | C | 65.73 | 70.508 | 15.458 |
| 3787 | LEU463 | O | 66.175 | 71.405 | 16.189 |
| 3788 | LEU464 | N | 65.166 | 70.733 | 14.279 |
| 3789 | LEU464 | CA | 65.273 | 72.018 | 13.58 |
| 3790 | LEU464 | CB | 65.297 | 71.738 | 12.082 |
| 3791 | LEU464 | CG | 66.441 | 70.812 | 11.691 |
| 3792 | LEU464 | CD1 | 66.343 | 70.429 | 10.221 |
| 3793 | LEU464 | CD2 | 67.794 | 71.443 | 11.994 |
| 3794 | LEU464 | C | 64.117 | 72.977 | 13.866 |
| 3795 | LEU464 | O | 64.02 | 74.013 | 13.199 |
| 3796 | VAL465 | N | 63.203 | 72.613 | 14.749 |
| 3797 | VAL465 | CA | 62.09 | 73.516 | 15.046 |
| 3798 | VAL465 | CB | 60.939 | 72.724 | 15.657 |
| 3799 | VAL465 | CG1 | 59.812 | 73.638 | 16.121 |
| 3800 | VAL465 | CG2 | 60.411 | 71.698 | 14.663 |
| 3801 | VAL465 | C | 62.554 | 74.616 | 15.99 |
| 3802 | VAL465 | O | 62.85 | 74.373 | 17.164 |
| 3803 | THR466 | N | 62.584 | 75.826 | 15.461 |
| 3804 | THR466 | CA | 63.041 | 76.984 | 16.226 |
| 3805 | THR466 | CB | 63.764 | 77.95 | 15.292 |
| 3806 | THR466 | OG1 | 62.851 | 78.394 | 14.299 |
| 3807 | THR466 | CG2 | 64.937 | 77.28 | 14.589 |
| 3808 | THR466 | C | 61.87 | 77.695 | 16.888 |
| 3809 | THR466 | O | 62.048 | 78.374 | 17.91 |
| 3810 | HIS467 | N | 60.676 | 77.462 | 16.372 |
| 3811 | HIS467 | CA | 59.482 | 78.061 | 16.976 |
| 3812 | HIS467 | CB | 58.976 | 79.183 | 16.077 |
| 3813 | HIS467 | CG | 60.007 | 80.268 | 15.83 |
| 3814 | HIS467 | ND1 | 60.513 | 81.111 | 16.749 |
| 3815 | HIS467 | CE1 | 61.412 | 81.926 | 16.163 |
| 3816 | HIS467 | NE2 | 61.474 | 81.591 | 14.855 |
| 3817 | HIS467 | CD2 | 60.613 | 80.573 | 14.634 |
| 3818 | HIS467 | C | 58.401 | 77.009 | 17.17 |
| 3819 | HIS467 | O | 57.755 | 76.571 | 16.209 |
| 3820 | LEU468 | N | 58.212 | 76.615 | 18.416 |
| 3821 | LEU468 | CA | 57.249 | 75.559 | 18.735 |
| 3822 | LEU468 | CB | 57.956 | 74.512 | 19.588 |
| 3823 | LEU468 | CG | 57.095 | 73.282 | 19.84 |
| 3824 | LEU468 | CD1 | 56.61 | 72.671 | 18.532 |
| 3825 | LEU468 | CD2 | 57.859 | 72.248 | 20.657 |
| 3826 | LEU468 | C | 56.048 | 76.138 | 19.473 |
| 3827 | LEU468 | O | 56.161 | 76.648 | 20.596 |
| 3828 | ASP469 | N | 54.904 | 76.068 | 18.817 |
| 3829 | ASP469 | CA | 53.677 | 76.613 | 19.393 |
| 3830 | ASP469 | CB | 52.998 | 77.475 | 18.337 |
| 3831 | ASP469 | OG | 51.761 | 78.159 | 18.908 |
| 3832 | ASP469 | OD1 | 51.666 | 78.263 | 20.123 |
| 3833 | ASP469 | OD2 | 50.892 | 78.489 | 18.114 |
| 3834 | ASP469 | C | 52.753 | 75.49 | 19.853 |
| 3835 | ASP469 | O | 51.954 | 74.961 | 19.076 |
| 3836 | LEU470 | N | 52.815 | 75.224 | 21.145 |
| 3837 | LEU470 | CA | 52.035 | 74.18 | 21.815 |
| 3838 | LEU470 | CB | 52.951 | 73.423 | 22.767 |
| 3839 | LEU470 | CG | 54.147 | 72.799 | 22.071 |
| 3840 | LEU470 | CD1 | 55.11 | 72.226 | 23.102 |
| 3841 | LEU470 | CD2 | 53.703 | 71.727 | 21.084 |
| 3842 | LEU470 | C | 50.929 | 74.771 | 22.682 |
| 3843 | LEU470 | O | 50.43 | 74.073 | 23.574 |
| 3844 | SER471 | N | 50.691 | 76.064 | 22.558 |
| 3845 | SER471 | CA | 49.681 | 76.727 | 23.391 |
| 3846 | SER471 | CB | 49.627 | 78.201 | 23.015 |
| 3847 | SER471 | OG | 49.205 | 78.281 | 21.661 |
| 3848 | SER471 | C | 48.289 | 76.121 | 23.23 |
| 3849 | SER471 | O | 47.916 | 75.653 | 22.148 |
| 3850 | HIS472 | N | 47.573 | 76.091 | 24.342 |
| 3851 | HIS472 | CA | 46.179 | 75.632 | 24.401 |
| 3852 | HIS472 | CB | 45.31 | 76.469 | 23.47 |
| 3853 | HIS472 | CG | 45.168 | 77.919 | 23.894 |
| 3854 | HIS472 | ND1 | 44.186 | 78.428 | 24.66 |
| 3855 | HIS472 | CE1 | 44.389 | 79.751 | 24.824 |
| 3856 | HIS472 | NE2 | 45.511 | 80.081 | 24.146 |
| 3857 | HIS472 | CD2 | 46 | 78.963 | 23.563 |
| 3858 | HIS472 | C | 46.059 | 74.15 | 24.076 |
| 3859 | HIS472 | O | 45.613 | 73.764 | 22.986 |
| 3860 | ASN473 | N | 46.572 | 73.354 | 24.997 |
| 3861 | ASN473 | CA | 46.5 | 71.89 | 24.923 |
| 3862 | ASN473 | CB | 47.777 | 71.334 | 24.291 |
| 3863 | ASN473 | CG | 47.782 | 71.539 | 22.778 |
| 3864 | ASN473 | OD1 | 46.778 | 71.289 | 22.105 |
| 3865 | ASN473 | ND2 | 48.906 | 71.975 | 22.25 |
| 3866 | ASN473 | C | 46.304 | 71.314 | 26.327 |
| 3867 | ASN473 | O | 46.094 | 72.054 | 27.296 |
| 3868 | ARG474 | N | 46.329 | 69.995 | 26.417 |
| 3869 | ARG474 | CA | 46.148 | 69.308 | 27.699 |
| 3870 | ARG474 | CB | 44.999 | 68.311 | 27.603 |
| 3871 | ARG474 | CG | 43.67 | 69.007 | 27.344 |
| 3872 | ARG474 | CD | 42.499 | 68.06 | 27.573 |
| 3873 | ARG474 | NE | 42.597 | 66.86 | 26.73 |
| 3874 | ARG474 | CZ | 41 .575 | 66.021 | 26.551 |
| 3875 | ARG474 | NH1 | 40.408 | 66.253 | 27.156 |
| 3876 | ARG474 | NH2 | 41.719 | 64.947 | 25.772 |
| 3877 | ARG474 | C | 47.41 | 68.576 | 28.15 |
| 3878 | ARG474 | O | 47.32 | 67.676 | 28.994 |
| 3879 | LEU475 | N | 48.55 | 68.945 | 27.583 |
| 3880 | LEU475 | CA | 49.838 | 68.334 | 27.944 |
| 3881 | LEU475 | CB | 50.949 | 69.109 | 27.239 |
| 3882 | LEU475 | OG | 50.74 | 69.225 | 25.732 |
| 3883 | LEU475 | CD1 | 51.635 | 70.307 | 25.137 |
| 3884 | LEU475 | CD2 | 50.967 | 67.894 | 25.032 |
| 3885 | LEU475 | C | 50.054 | 68.477 | 29.442 |
| 3886 | LEU475 | O | 49.805 | 69.558 | 29.982 |
| 3887 | ARG476 | N | 50.469 | 67.411 | 30.108 |
| 3888 | ARG476 | CA | 50.679 | 67.473 | 31.567 |
| 3889 | ARG476 | CB | 50.319 | 66.118 | 32.169 |
| 3890 | ARG476 | CG | 48.859 | 65.738 | 31.941 |
| 3891 | ARG476 | CD | 47.905 | 66.681 | 32.666 |
| 3892 | ARG476 | NE | 48.198 | 66.735 | 34.108 |
| 3893 | ARG476 | CZ | 47.448 | 66.136 | 35.036 |
| 3894 | ARG476 | NH1 | 47.778 | 66.235 | 36.325 |
| 3895 | ARG476 | NH2 | 46.364 | 65.444 | 34.676 |
| 3896 | ARG476 | C | 52.128 | 67.786 | 31.931 |
| 3897 | ARG476 | O | 52.436 | 68.26 | 33.036 |
| 3898 | THR477 | N | 53.001 | 67.589 | 30.962 |
| 3899 | THR477 | CA | 54.429 | 67.822 | 31.164 |
| 3900 | THR477 | CB | 55.01 | 66.64 | 31.944 |
| 3901 | THR477 | OG1 | 56.419 | 66.806 | 32.054 |
| 3902 | THR477 | CG2 | 54.756 | 65.309 | 31.243 |
| 3903 | THR477 | C | 55.136 | 67.94 | 29.823 |
| 3904 | THR477 | O | 54.678 | 67.383 | 28.818 |
| 3905 | LEU478 | N | 56.181 | 68.746 | 29.805 |
| 3906 | LEU478 | CA | 57.105 | 68.751 | 28.676 |
| 3907 | LEU478 | CB | 57.807 | 70.1 | 28.61 |
| 3908 | LEU478 | CG | 56.811 | 71.191 | 28.235 |
| 3909 | LEU478 | CD1 | 57.427 | 72.581 | 28.333 |
| 3910 | LEU478 | CD2 | 56.245 | 70.954 | 26.839 |
| 3911 | LEU478 | C | 58.102 | 67.618 | 28.882 |
| 3912 | LEU478 | O | 58.938 | 67.657 | 29.798 |
| 3913 | PRO479 | N | 57.987 | 66.617 | 28.023 |
| 3914 | PRO479 | CA | 58.706 | 65.348 | 28.189 |
| 3915 | PRO479 | CB | 58.109 | 64.426 | 27.167 |
| 3916 | PRO479 | CG | 57.115 | 65.19 | 26.31 |
| 3917 | PRO479 | CD | 57.077 | 66.597 | 26.874 |
| 3918 | PRO479 | C | 60.195 | 65.546 | 27.952 |
| 3919 | PRO479 | O | 60.573 | 66.488 | 27.251 |
| 3920 | PRO480 | N | 61.03 | 64.668 | 28.491 |
| 3921 | PRO480 | CA | 62.492 | 64.822 | 28.366 |
| 3922 | PRO480 | CB | 63.073 | 63.768 | 29.258 |
| 3923 | PRO480 | CG | 61.952 | 62.923 | 29.843 |
| 3924 | PRO480 | CD | 60.655 | 63.52 | 29.324 |
| 3925 | PRO480 | C | 63.038 | 64.685 | 26.933 |
| 3926 | PRO480 | O | 64.095 | 65.252 | 26.635 |
| 3927 | ALA481 | N | 62.218 | 64.188 | 26.016 |
| 3928 | ALA481 | CA | 62.581 | 64.126 | 24.595 |
| 3929 | ALA481 | CB | 61.715 | 63.072 | 23.917 |
| 3930 | ALA481 | C | 62.422 | 65.472 | 23.873 |
| 3931 | ALA481 | O | 62.878 | 65.607 | 22.731 |
| 3932 | LEU482 | N | 61.965 | 66.494 | 24.587 |
| 3933 | LEU482 | CA | 61.858 | 67.849 | 24.036 |
| 3934 | LEU482 | CB | 60.922 | 68.644 | 24.941 |
| 3935 | LEU482 | OG | 60.638 | 70.043 | 24.412 |
| 3936 | LEU482 | CD1 | 59.822 | 69.977 | 23.127 |
| 3937 | LEU482 | CD2 | 59.901 | 70.869 | 25.457 |
| 3938 | LEU482 | C | 63.233 | 68.525 | 23.975 |
| 3939 | LEU482 | O | 63.453 | 69.369 | 23.098 |
| 3940 | ALA483 | N | 64.208 | 67.91 | 24.635 |
| 3941 | ALA483 | CA | 65.611 | 68.335 | 24.546 |
| 3942 | ALA483 | CB | 66.366 | 67.787 | 25.752 |
| 3943 | ALA483 | C | 66.3 | 67.884 | 23.249 |
| 3944 | ALA483 | O | 67.48 | 68.185 | 23.039 |
| 3945 | ALA484 | N | 65.571 | 67.196 | 22.378 |
| 3946 | ALA484 | CA | 66.07 | 66.89 | 21.037 |
| 3947 | ALA484 | CB | 65.395 | 65.619 | 20.535 |
| 3948 | ALA484 | C | 65.783 | 68.042 | 20.068 |
| 3949 | ALA484 | O | 66.313 | 68.057 | 18.951 |
| 3950 | LEU485 | N | 65.005 | 69.016 | 20.515 |
| 3951 | LEU485 | CA | 64.757 | 70.242 | 19.745 |
| 3952 | LEU485 | CB | 63.353 | 70.788 | 20.03 |
| 3953 | LEU485 | CG | 62.198 | 70.015 | 19.394 |
| 3954 | LEU485 | CD1 | 61.754 | 68.803 | 20.21 |
| 3955 | LEU485 | CD2 | 61.006 | 70.947 | 19.212 |
| 3956 | LEU485 | C | 65.757 | 71.308 | 20.17 |
| 3957 | LEU485 | O | 65.375 | 72.318 | 20.767 |
| 3958 | ARG486 | N | 66.998 | 71.161 | 19.738 |
| 3959 | ARG486 | CA | 68.063 | 72.03 | 20.249 |
| 3960 | ARG486 | CB | 69.383 | 71.283 | 20.135 |
| 3961 | ARG486 | CG | 69.268 | 69.908 | 20.783 |
| 3962 | ARG486 | CD | 70.612 | 69.196 | 20.856 |
| 3963 | ARG486 | NE | 71.472 | 69.773 | 21.902 |
| 3964 | ARG486 | CZ | 72.658 | 70.339 | 21.667 |
| 3965 | ARG486 | NH1 | 73.069 | 70.535 | 20.413 |
| 3966 | ARG486 | NH2 | 73.395 | 70.785 | 22.687 |
| 3967 | ARG486 | C | 68.152 | 73.375 | 19.53 |
| 3968 | ARG486 | O | 68.753 | 74.316 | 20.068 |
| 3969 | CYS487 | N | 67.447 | 73.5 | 18.414 |
| 3970 | CYS487 | CA | 67.363 | 74.78 | 17.702 |
| 3971 | CYS487 | CB | 67.248 | 74.499 | 16.209 |
| 3972 | CYS487 | SG | 68.608 | 73.545 | 15.499 |
| 3973 | CYS487 | C | 66.159 | 75.607 | 18.155 |
| 3974 | CYS487 | O | 65.956 | 76.718 | 17.649 |
| 3975 | LEU488 | N | 65.386 | 75.07 | 19.088 |
| 3976 | LEU488 | CA | 64.205 | 75.758 | 19.613 |
| 3977 | LEU488 | CB | 63.524 | 74.798 | 20.58 |
| 3978 | LEU488 | CG | 62.208 | 75.339 | 21.113 |
| 3979 | LEU488 | OD1 | 61.272 | 75.661 | 19.96 |
| 3980 | LEU488 | CD2 | 61.568 | 74.334 | 22.062 |
| 3981 | LEU488 | C | 64.603 | 77.03 | 20.344 |
| 3982 | LEU488 | O | 65.341 | 76.979 | 21 .329 |
| 3983 | GLU489 | N | 64.125 | 78.153 | 19.836 |
| 3984 | GLU489 | CA | 64.426 | 79.46 | 20.409 |
| 3985 | GLU489 | CB | 64.814 | 80.388 | 19.268 |
| 3986 | GLU489 | CG | 66.055 | 79.878 | 18.549 |
| 3987 | GLU489 | CD | 66.25 | 80.642 | 17.248 |
| 3988 | GLU489 | OE1 | 65.244 | 80.885 | 16.591 |
| 3989 | GLU489 | OE2 | 67.394 | 80.786 | 16.837 |
| 3990 | GLU489 | C | 63.211 | 80.022 | 21.123 |
| 3991 | GLU489 | O | 63.337 | 80.694 | 22.157 |
| 3992 | VAL490 | N | 62.042 | 79.715 | 20.59 |
| 3993 | VAL490 | CA | 60.796 | 80.149 | 21.232 |
| 3994 | VAL490 | CB | 60.09 | 81.171 | 20.343 |
| 3995 | VAL490 | CG1 | 58.719 | 81.55 | 20.896 |
| 3996 | VAL490 | CG2 | 60.943 | 82.421 | 20.151 |
| 3997 | VAL490 | C | 59.88 | 78.96 | 21 .501 |
| 3998 | VAL490 | O | 59.407 | 78.289 | 20.572 |
| 3999 | LEU491 | N | 59.678 | 78.691 | 22.779 |
| 4000 | LEU491 | CA | 58.761 | 77.633 | 23.199 |
| 4001 | LEU491 | CB | 59.472 | 76.723 | 24.195 |
| 40D2 | LEU491 | CG | 58.585 | 75.567 | 24.651 |
| 4003 | LEU491 | OD1 | 58.036 | 74.783 | 23.465 |
| 4004 | LEU491 | CD2 | 59.343 | 74.641 | 25.596 |
| 4005 | LEU491 | C | 57.516 | 78.244 | 23.833 |
| 4006 | LEU491 | O | 57.55 | 78.765 | 24.956 |
| 4007 | GLN492 | N | 56.434 | 78.204 | 23.077 |
| 4008 | GLN492 | CA | 55.144 | 78.695 | 23.56 |
| 4009 | GLN492 | CB | 54.456 | 79.441 | 22.414 |
| 4010 | GLN492 | CG | 52.988 | 79.785 | 22.685 |
| 4011 | GLN492 | CD | 52.811 | 80.661 | 23.922 |
| 4012 | GLN492 | OE1 | 53.034 | 80.212 | 25.051 |
| 4013 | GLN492 | NE2 | 52.348 | 81.877 | 23.698 |
| 4014 | GLN492 | C | 54.297 | 77.519 | 24.029 |
| 4015 | GLN492 | O | 53.707 | 76.812 | 23.21 |
| 4016 | ALA493 | N | 54.238 | 77.32 | 25.332 |
| 4017 | ALA493 | CA | 53.495 | 76.192 | 25.891 |
| 4018 | ALA493 | CB | 54.465 | 75.248 | 26.589 |
| 4019 | ALA493 | C | 52.405 | 76.656 | 26.856 |
| 4020 | ALA493 | O | 51.894 | 75.858 | 27.656 |
| 4021 | SER494 | N | 52.066 | 77.931 | 26.779 |
| 4022 | SER494 | CA | 51.014 | 78.512 | 27.622 |
| 4023 | SER494 | CB | 50.852 | 79.983 | 27.268 |
| 4024 | SER494 | OG | 52.058 | 80.648 | 27.622 |
| 4025 | SER494 | C | 49.669 | 77.813 | 27.464 |
| 4026 | SER494 | O | 49.409 | 77.111 | 26.476 |
| 4027 | ASP495 | N | 48.849 | 77.984 | 28.487 |
| 4028 | ASP495 | CA | 47.498 | 77.419 | 28.563 |
| 4029 | ASP495 | CB | 46.611 | 78.07 | 27.511 |
| 4030 | ASP495 | OG | 46.546 | 79.577 | 27.752 |
| 4031 | ASP495 | OD1 | 45.696 | 79.991 | 28.527 |
| 4032 | ASP495 | OD2 | 47.301 | 80.289 | 27.101 |
| 4033 | ASP495 | C | 47.556 | 75.913 | 28.397 |
| 4034 | ASP495 | O | 47.255 | 75.368 | 27.325 |
| 4035 | ASN496 | N | 48.137 | 75.292 | 29.405 |
| 4036 | ASN496 | CA | 48.326 | 73.839 | 29.425 |
| 4037 | ASN496 | CB | 49.661 | 73.468 | 28.783 |
| 4038 | ASN496 | CG | 49.534 | 73.059 | 27.318 |
| 4039 | ASN496 | OD1 | 49.184 | 71.912 | 27.013 |
| 4040 | ASN496 | ND2 | 49.944 | 73.957 | 26.442 |
| 4041 | ASN496 | C | 48.339 | 73.337 | 30.858 |
| 4042 | ASN496 | O | 48.654 | 74.079 | 31.796 |
| 4043 | ALA497 | N | 48.235 | 72.026 | 30.98 |
| 4044 | ALA497 | CA | 48.265 | 71.371 | 32.292 |
| 4045 | ALA497 | CB | 47.429 | 70.102 | 32.209 |
| 4046 | ALA497 | C | 49.689 | 71.045 | 32.759 |
| 4047 | ALA497 | O | 49.879 | 70.412 | 33.803 |
| 4048 | 1LEA498 | N | 50.665 | 71.479 | 31.974 |
| 4049 | 1LEA498 | CA | 52.087 | 71.271 | 32.253 |
| 4050 | 1LEA498 | CB | 52.887 | 71.968 | 31.159 |
| 4051 | 1LEA498 | CG2 | 54.384 | 71.81 | 31.393 |
| 4052 | 1LEA498 | CG1 | 52.511 | 71.413 | 29.794 |
| 4053 | 1LEA498 | CD1 | 53.219 | 72.166 | 28.676 |
| 4054 | 1LEA498 | C | 52.511 | 71.804 | 33.613 |
| 4055 | 1LEA498 | O | 52.459 | 73.011 | 33.887 |
| 4056 | GLU499 | N | 52.842 | 70.855 | 34.471 |
| 4057 | GLU499 | CA | 53.388 | 71.137 | 35.796 |
| 4058 | GLU499 | CB | 52.518 | 70.418 | 36.822 |
| 4059 | GLU499 | CG | 52.157 | 69.009 | 36.367 |
| 4060 | GLU499 | CD | 51.21 | 68.36 | 37.371 |
| 4061 | GLU499 | OE1 | 50.031 | 68.681 | 37.337 |
| 4062 | GLU499 | OE2 | 51.673 | 67.503 | 38.112 |
| 4063 | GLU499 | C | 54.845 | 70.692 | 35.888 |
| 4064 | GLU499 | O | 55.54 | 70.982 | 36.869 |
| 4065 | SER500 | N | 55.296 | 69.995 | 34.858 |
| 4066 | SER50O | CA | 56.692 | 69.547 | 34.802 |
| 4067 | SER500 | CB | 56.703 | 68.03 | 34.895 |
| 4068 | SER500 | OG | 57.999 | 67.589 | 34.523 |
| 4069 | SER500 | C | 57.389 | 69.998 | 33.521 |
| 4070 | SER500 | O | 56.949 | 69.678 | 32.41 |
| 4071 | LEU501 | N | 58.53 | 70.646 | 33.687 |
| 4072 | LEU501 | CA | 59.279 | 71.207 | 32.549 |
| 4073 | LEU501 | CB | 59.611 | 72.655 | 32.889 |
| 4074 | LEU501 | CG | 58.354 | 73.464 | 33.183 |
| 4075 | LEU501 | CO1 | 58.7 | 74.809 | 33.807 |
| 4076 | LEU501 | CD2 | 57.506 | 73.644 | 31.93 |
| 4077 | LEU501 | C | 60.586 | 70.457 | 32.293 |
| 4078 | LEU501 | O | 61.601 | 71.081 | 31.954 |
| 4079 | ASP502 | N | 60.513 | 69.137 | 32.243 |
| 4080 | ASP502 | CA | 61.749 | 68.338 | 32.274 |
| 4081 | ASP502 | CB | 61.42 | 66.89 | 32.626 |
| 4082 | ASP502 | CG | 60.866 | 66.765 | 34.044 |
| 4083 | ASP502 | OD1 | 61.01 | 67.71 | 34.811 |
| 4084 | ASP502 | OD2 | 60.208 | 65.767 | 34.301 |
| 4085 | ASP502 | C | 62.507 | 68.356 | 30.953 |
| 4086 | ASP502 | O | 63.729 | 68.541 | 30.966 |
| 4087 | GLY503 | N | 61.778 | 68.477 | 29.856 |
| 4088 | GLY503 | CA | 62.409 | 68.487 | 28.532 |
| 4089 | GLY503 | C | 62.806 | 69.873 | 28.037 |
| 4090 | GLY503 | O | 63.112 | 70.041 | 26.853 |
| 4091 | VAL504 | N | 62.773 | 70.853 | 28.925 |
| 4092 | VAL504 | CA | 63.266 | 72.183 | 28.588 |
| 4093 | VAL504 | CB | 62.384 | 73.202 | 29.299 |
| 4094 | VAL504 | CG1 | 62.736 | 74.624 | 28.889 |
| 4095 | VAL504 | CG2 | 60.913 | 72.933 | 29.014 |
| 4096 | VAL504 | C | 64.716 | 72.303 | 29.055 |
| 4097 | VAL504 | O | 65.472 | 73.164 | 28.588 |
| 4098 | THR505 | N | 65.119 | 71.341 | 29.868 |
| 4099 | THR505 | CA | 66.477 | 71.301 | 30.412 |
| 4100 | THR505 | CB | 66.507 | 70.206 | 31.477 |
| 4101 | THR505 | OG1 | 65.481 | 70.494 | 32.418 |
| 4102 | THR505 | CG2 | 67.827 | 70.125 | 32.238 |
| 4103 | THR505 | C | 67.487 | 71.029 | 29.296 |
| 4104 | THR505 | O | 67.307 | 70.116 | 28.481 |
| 4105 | ASN506 | N | 68.575 | 71.782 | 29.345 |
| 4106 | ASN506 | CA | 69.638 | 71.783 | 28.332 |
| 4107 | ASN506 | CB | 70.383 | 70.451 | 28.36 |
| 4108 | ASN506 | CG | 70.893 | 70.14 | 29.767 |
| 4109 | ASN506 | OD1 | 71.343 | 71.022 | 30.507 |
| 4110 | ASN506 | ND2 | 70.741 | 68.884 | 30.143 |
| 4111 | ASN506 | C | 69.112 | 72.058 | 26.927 |
| 4112 | ASN506 | O | 69.24 | 71.221 | 26.025 |
| 4113 | LEU507 | N | 68.481 | 73.209 | 26.761 |
| 4114 | LEU507 | CA | 68.08 | 73.652 | 25.42 |
| 4115 | LEU507 | CB | 66.586 | 73.948 | 25.365 |
| 4116 | LEU507 | CG | 65.771 | 72.667 | 25.226 |
| 4117 | LEU507 | OD1 | 64.283 | 72.984 | 25.124 |
| 4118 | LEU507 | CD2 | 66.222 | 71.883 | 23.998 |
| 4119 | LEU507 | C | 68.878 | 74.882 | 25.017 |
| 4120 | LEU507 | O | 68.574 | 76.005 | 25.44 |
| 4121 | PRO508 | N | 69.789 | 74.669 | 24.08 |
| 4122 | PRO508 | CA | 70.867 | 75.63 | 23.829 |
| 4123 | PRO508 | CB | 71.738 | 74.992 | 22.792 |
| 4124 | PRO508 | CG | 71.19 | 73.616 | 22.457 |
| 4125 | PRO508 | CD | 69.977 | 73.414 | 23.349 |
| 4126 | PRO508 | C | 70.347 | 76.976 | 23.349 |
| 4127 | PRO508 | O | 70.467 | 77.97 | 24.076 |
| 4128 | ARG509 | N | 69.544 | 76.932 | 22.299 |
| 4129 | ARG509 | CA | 69.041 | 78.155 | 21.673 |
| 4130 | ARG509 | CB | 68.834 | 77.895 | 20.185 |
| 4131 | ARG509 | CG | 70.126 | 77.504 | 19.475 |
| 4132 | ARG509 | CD | 71.213 | 78.563 | 19.64 |
| 4133 | ARG509 | NE | 70.76 | 79.888 | 19.189 |
| 4134 | ARG509 | CZ | 71.43 | 80.621 | 18.299 |
| 4135 | ARG509 | NH1 | 72.545 | 80.142 | 17.745 |
| 4136 | ARG509 | NH2 | 70.974 | 81.825 | 17.95 |
| 4137 | ARG509 | C | 67.734 | 78.682 | 22.262 |
| 4138 | ARG509 | O | 67.181 | 79.628 | 21.692 |
| 4139 | LEU510 | N | 67.259 | 78.127 | 23.367 |
| 4140 | LEU510 | CA | 65.961 | 78.558 | 23.9 |
| 4141 | LEU510 | CB | 65.427 | 77.508 | 24.863 |
| 4142 | LEU510 | CG | 64.004 | 77.848 | 25.288 |
| 4143 | LEU510 | CD1 | 63.077 | 77.883 | 24.081 |
| 4144 | LEU510 | CD2 | 63.489 | 76.855 | 26.317 |
| 4145 | LEU510 | C | 66.092 | 79.89 | 24.619 |
| 4146 | LEU510 | O | 66.653 | 79.959 | 25.717 |
| 4147 | GLN511 | N | 65.528 | 80.919 | 24.009 |
| 4148 | GLN511 | CA | 65.633 | 82.285 | 24.512 |
| 4149 | GLN511 | CB | 65.863 | 83.187 | 23.306 |
| 4150 | GLN511 | CG | 66.983 | 82.638 | 22.434 |
| 4151 | GLN511 | CD | 67.133 | 83.451 | 21.156 |
| 4152 | GLN511 | OE1 | 66.602 | 83.086 | 20.099 |
| 4153 | GLN511 | NE2 | 67.869 | 84.543 | 21.27 |
| 4154 | GLN511 | C | 64.35 | 82.718 | 25.197 |
| 4155 | GLN511 | O | 64.379 | 83.493 | 26.166 |
| 4156 | GLU512 | N | 63.24 | 82.213 | 24.684 |
| 4157 | GLU512 | CA | 61.92 | 82.567 | 25.219 |
| 4158 | GLU512 | CB | 61.133 | 83.302 | 24.139 |
| 4159 | GLU512 | CG | 61.832 | 84.579 | 23.687 |
| 4160 | GLU512 | CD | 60.978 | 85.298 | 22.648 |
| 4161 | GLU512 | OE1 | 59.764 | 85.178 | 22.73 |
| 4162 | GLU512 | OE2 | 61.557 | 85.927 | 21.773 |
| 4163 | GLU512 | C | 61.124 | 81.339 | 25.647 |
| 4164 | GLU512 | O | 60.82 | 80.459 | 24.828 |
| 4165 | LEU513 | N | 60.78 | 81.309 | 26.922 |
| 4166 | LEU513 | CA | 59.912 | 80.259 | 27.46 |
| 4167 | LEU513 | CB | 60.653 | 79.566 | 28.597 |
| 4168 | LEU513 | CG | 59.894 | 78.356 | 29.126 |
| 4169 | LEU513 | CD1 | 59.528 | 77.399 | 28 |
| 4170 | LEU513 | CD2 | 60.704 | 77.635 | 30.195 |
| 4171 | LEU513 | C | 58.598 | 80.873 | 27.952 |
| 4172 | LEU513 | O | 58.562 | 81.621 | 28.942 |
| 4173 | LEU514 | N | 57.536 | 80.58 | 27.22 |
| 4174 | LEU514 | CA | 56.215 | 81.147 | 27.51 |
| 4175 | LEU514 | CB | 55.621 | 81.625 | 26.192 |
| 4176 | LEU514 | CG | 56.521 | 82.643 | 25.5 |
| 4177 | LEU514 | CD1 | 56.082 | 82.886 | 24.061 |
| 4178 | LEU514 | CD2 | 56.58 | 83.952 | 26.279 |
| 4179 | LEU514 | C | 55.291 | 80.109 | 28.145 |
| 4180 | LEU514 | O | 54.83 | 79.171 | 27.482 |
| 4181 | LEU515 | N | 55.004 | 80.318 | 29.418 |
| 4182 | LEU515 | CA | 54.173 | 79.41 | 30.216 |
| 4183 | LEU515 | CB | 55.067 | 78.629 | 31.174 |
| 4184 | LEU515 | CG | 56.082 | 77.739 | 30.469 |
| 4185 | LEU515 | CD1 | 57.131 | 77.248 | 31.456 |
| 4186 | LEU515 | CD2 | 55.401 | 76.57 | 29.772 |
| 4187 | LEU515 | C | 53.178 | 80.19 | 31.073 |
| 4188 | LEU515 | O | 53.331 | 80.243 | 32.3 |
| 4189 | CYS516 | N | 52.222 | 80.833 | 30.427 |
| 4190 | CYS516 | CA | 51.149 | 81.531 | 31.139 |
| 4191 | CYS516 | CB | 50.745 | 82.777 | 30.368 |
| 4192 | CYS516 | SG | 51.989 | 84.078 | 30.291 |
| 4193 | CYS516 | C | 49.938 | 80.626 | 31.271 |
| 4194 | CYS516 | O | 49.491 | 80.04 | 30.277 |
| 4195 | ASN517 | N | 49.37 | 80.603 | 32.462 |
| 4196 | ASN517 | CA | 48.242 | 79.724 | 32.794 |
| 4197 | ASN517 | CB | 47.002 | 80.136 | 32.012 |
| 4198 | ASN517 | CG | 46.592 | 81.54 | 32.448 |
| 4199 | ASN517 | OD1 | 46.73 | 82.51 | 31.693 |
| 4200 | ASN517 | ND2 | 46.151 | 81.643 | 33.691 |
| 4201 | ASN517 | C | 48.611 | 78.266 | 32.55 |
| 4202 | ASN517 | O | 48.154 | 77.607 | 31.603 |
| 4203 | ASN518 | N | 49.586 | 77.85 | 33.333 |
| 4204 | ASN518 | CA | 50.064 | 76.47 | 33.374 |
| 4205 | ASN518 | CB | 51.481 | 76.403 | 32.809 |
| 4206 | ASN518 | CG | 51.508 | 76.636 | 31.298 |
| 4207 | ASN518 | OD1 | 51.33 | 77.758 | 30.805 |
| 4208 | ASN518 | ND2 | 51.838 | 75.577 | 30.584 |
| 4209 | ASN518 | C | 50.051 | 76.009 | 34.828 |
| 4210 | ASN518 | O | 50.002 | 76.838 | 35.745 |
| 4211 | ARG519 | N | 50.239 | 74.72 | 35.048 |
| 4212 | ARG519 | CA | 50.128 | 74.163 | 36.408 |
| 4213 | ARG519 | CB | 49.533 | 72.764 | 36.339 |
| 4214 | ARG519 | CG | 48.092 | 72.818 | 35.85 |
| 4215 | ARG519 | CD | 47.424 | 71.453 | 35.94 |
| 4216 | ARG519 | NE | 46.05 | 71.52 | 35.421 |
| 4217 | ARG519 | CZ | 45.238 | 70.462 | 35.365 |
| 4218 | ARG519 | NH1 | 45.655 | 69.278 | 35.819 |
| 4219 | ARG519 | NH2 | 44.005 | 70.592 | 34.87 |
| 4220 | ARG519 | C | 51.435 | 74.133 | 37.206 |
| 4221 | ARG519 | O | 51.649 | 73.204 | 37.995 |
| 4222 | LEU520 | N | 52.29 | 75.124 | 37.012 |
| 4223 | LEU520 | CA | 53.525 | 75.223 | 37.805 |
| 4224 | LEU520 | CB | 54.526 | 76.193 | 37.164 |
| 4225 | LEU520 | CG | 55.264 | 75.657 | 35.931 |
| 4226 | LEU520 | CO1 | 55.652 | 74.197 | 36.11 |
| 4227 | LEU520 | CD2 | 54.496 | 75.833 | 34.625 |
| 4228 | LEU520 | C | 53.167 | 75.721 | 39.205 |
| 4229 | LEU520 | O | 52.919 | 76.918 | 39.402 |
| 4230 | GLN521 | N | 53.133 | 74.803 | 40.157 |
| 4231 | GLN521 | CA | 52.664 | 75.127 | 41.508 |
| 4232 | GLN521 | CB | 51.992 | 73.889 | 42.088 |
| 4233 | GLN521 | CG | 51.458 | 74.162 | 43.49 |
| 4234 | GLN521 | CD | 51.43 | 72.869 | 44.296 |
| 4235 | GLN521 | OE1 | 52.179 | 71.928 | 44.002 |
| 4236 | GLN521 | NE2 | 50.653 | 72.88 | 45.364 |
| 4237 | GLN521 | C | 53.789 | 75.528 | 42.451 |
| 4238 | GLN521 | O | 53.612 | 76.421 | 43.286 |
| 4239 | GLN522 | N | 54.937 | 74.891 | 42.302 |
| 4240 | GLN522 | CA | 56.071 | 75.184 | 43.184 |
| 4241 | GLN522 | CB | 56.408 | 73.917 | 43.964 |
| 4242 | GLN522 | CG | 55.252 | 73.516 | 44.873 |
| 4243 | GLN522 | CD | 55.566 | 72.209 | 45.588 |
| 4244 | GLN522 | QE1 | 56.605 | 72.077 | 46.244 |
| 4245 | GLN522 | NE2 | 54.658 | 71.258 | 45.452 |
| 4246 | GLN522 | C | 57.284 | 75.65 | 42.388 |
| 4247 | GLN522 | O | 57.639 | 75.035 | 41.375 |
| 4248 | PRO523 | N | 57.988 | 76.636 | 42.926 |
| 4249 | PRO523 | CA | 59.095 | 77.289 | 42.204 |
| 4250 | PRO523 | CB | 59.418 | 78.507 | 43.017 |
| 4251 | PRO523 | CG | 58.592 | 78.503 | 44.293 |
| 4252 | PRO523 | CD | 57.696 | 77.28 | 44.212 |
| 4253 | PRO523 | C | 60.354 | 76.43 | 42.01 |
| 4254 | PRO523 | O | 61.154 | 76.728 | 41.114 |
| 4255 | ALA524 | N | 60.403 | 75.265 | 42.641 |
| 4256 | ALA524 | CA | 61.561 | 74.374 | 42.512 |
| 4257 | ALA524 | CB | 61.536 | 73.384 | 43.671 |
| 4258 | ALA524 | C | 61.59 | 73.608 | 41.186 |
| 4259 | ALA524 | O | 62.675 | 73.203 | 40.752 |
| 4260 | VAL525 | N | 60.492 | 73.637 | 40.441 |
| 4261 | VAL525 | CA | 60.462 | 72.988 | 39.123 |
| 4262 | VAL525 | CB | 59.019 | 72.595 | 38.8 |
| 4263 | VAL525 | CG1 | 58.13 | 73.818 | 38.611 |
| 4264 | VAL525 | CG2 | 58.932 | 71.688 | 37.574 |
| 4265 | VAL525 | C | 61.048 | 73.907 | 38.041 |
| 4266 | VAL525 | O | 61.329 | 73.46 | 36.923 |
| 4267 | LEU526 | N | 61.37 | 75.136 | 38.419 |
| 4268 | LEU526 | CA | 62.025 | 76.054 | 37.492 |
| 4269 | LEU526 | CB | 61.62 | 77.481 | 37.817 |
| 4270 | LEU526 | CG | 60.111 | 77.685 | 37.785 |
| 4271 | LEU526 | CD1 | 59.794 | 79.115 | 38.174 |
| 4272 | LEU526 | CD2 | 59.519 | 77.381 | 36.413 |
| 4273 | LEU526 | C | 63.539 | 75.946 | 37.611 |
| 4274 | LEU526 | O | 64.263 | 76.413 | 36.723 |
| 4275 | GLN527 | N | 64.01 | 75.219 | 38.611 |
| 4276 | GLN527 | CA | 65.456 | 75.042 | 38.776 |
| 4277 | GLN527 | CB | 65.743 | 74.292 | 40.07 |
| 4278 | GLN527 | CG | 67.21 | 74.447 | 40.453 |
| 4279 | GLN527 | CD | 67.511 | 75.927 | 40.674 |
| 4280 | GLN527 | QE1 | 66.909 | 76.559 | 41.55 |
| 4281 | GLN527 | NE2 | 68.394 | 76.469 | 39.851 |
| 4282 | GLN527 | C | 66.178 | 74.348 | 37.594 |
| 4283 | GLN527 | O | 67.216 | 74.898 | 37.198 |
| 4284 | PRO528 | N | 65.669 | 73.289 | 36.954 |
| 4285 | PRO528 | CA | 66.335 | 72.801 | 35.732 |
| 4286 | PRO528 | CB | 65.693 | 71.483 | 35.426 |
| 4287 | PRO528 | CG | 64.492 | 71.279 | 36.327 |
| 4288 | PRO528 | CD | 64.489 | 72.462 | 37.274 |
| 4289 | PRO528 | C | 66.252 | 73.717 | 34.499 |
| 4290 | PRO528 | O | 66.911 | 73.417 | 33.497 |
| 4291 | LEU529 | N | 65.597 | 74.867 | 34.591 |
| 4292 | LEU529 | CA | 65.547 | 75.81 | 33.468 |
| 4293 | LEU529 | CB | 64.289 | 76.664 | 33.556 |
| 4294 | LEU529 | CG | 63.017 | 75.834 | 33.646 |
| 4295 | LEU529 | CD1 | 61.809 | 76.753 | 33.732 |
| 4296 | LEU529 | CD2 | 62.873 | 74.884 | 32.466 |
| 4297 | LEU529 | C | 66.764 | 76.736 | 33.458 |
| 4298 | LEU529 | O | 67.027 | 77.39 | 32.441 |
| 4299 | ALA530 | N | 67.604 | 76.629 | 34.48 |
| 4300 | ALA530 | CA | 68.863 | 77.389 | 34.53 |
| 4301 | ALA530 | CB | 69.378 | 77.398 | 35.964 |
| 4302 | ALA530 | C | 69.94 | 76.796 | 33.615 |
| 4303 | ALA530 | O | 71.003 | 77.394 | 33.42 |
| 4304 | SER531 | N | 69.634 | 75.65 | 33.026 |
| 4305 | SER531 | CA | 70.508 | 75.019 | 32.037 |
| 4306 | SER531 | CB | 70.387 | 73.514 | 32.183 |
| 4307 | SER531 | OG | 69.087 | 73.16 | 31.741 |
| 4308 | SER531 | C | 70.136 | 75.409 | 30.603 |
| 4309 | SER531 | O | 70.537 | 74.707 | 29.668 |
| 4310 | CYS532 | N | 69.224 | 76.354 | 30.437 |
| 4311 | CYS532 | CA | 68.935 | 76.873 | 29.096 |
| 4312 | CYS532 | CB | 67.465 | 77.265 | 29.015 |
| 4313 | CYS532 | 5G | 66.289 | 75.98 | 29.486 |
| 4314 | CYS532 | C | 69.791 | 78.111 | 28.841 |
| 4315 | CYS532 | O | 69.453 | 79.207 | 29.302 |
| 4316 | PRO533 | N | 70.832 | 77.951 | 28.037 |
| 4317 | PRO533 | CA | 71.903 | 78.957 | 27.976 |
| 4318 | PRO533 | CB | 73.04 | 78.269 | 27.283 |
| 4319 | PRO533 | CG | 72.592 | 76.897 | 26.815 |
| 4320 | PRO533 | CD | 71.167 | 76.727 | 27.307 |
| 4321 | PRO533 | C | 71.533 | 80.243 | 27.232 |
| 4322 | PRO533 | O | 72.247 | 81.244 | 27.354 |
| 4323 | ARG534 | N | 70.419 | 80.241 | 26.519 |
| 4324 | ARG534 | CA | 69.964 | 81.447 | 25.829 |
| 4325 | ARG534 | CB | 69.591 | 81.054 | 24.409 |
| 4326 | ARG534 | CG | 70.392 | 81.812 | 23.359 |
| 4327 | ARG534 | CD | 71.884 | 81.554 | 23.509 |
| 4328 | ARG534 | NE | 72.624 | 82.107 | 22.367 |
| 4329 | ARG534 | CZ | 73.463 | 81.361 | 21.649 |
| 4330 | ARG534 | NH1 | 73.661 | 80.085 | 21.984 |
| 4331 | ARG534 | NH2 | 74.113 | 81.891 | 20.612 |
| 4332 | ARG534 | C | 68.745 | 82.076 | 26.504 |
| 4333 | ARG534 | O | 68.219 | 83.068 | 25.981 |
| 4334 | LEU535 | N | 68.321 | 81.533 | 27.638 |
| 4335 | LEU535 | CA | 67.025 | 81.908 | 28.222 |
| 4336 | LEU535 | CB | 66.612 | 80.841 | 29.228 |
| 4337 | LEU535 | CG | 65.157 | 81.004 | 29.655 |
| 4338 | LEU535 | CD1 | 64.234 | 80.871 | 28.45 |
| 4339 | LEU535 | CD2 | 64.784 | 79.983 | 30.724 |
| 4340 | LEU535 | C | 67.054 | 83.266 | 28.908 |
| 4341 | LEU535 | O | 67.527 | 83.415 | 30.041 |
| 4342 | VAL536 | N | 66.46 | 84.232 | 28.232 |
| 4343 | VAL536 | CA | 66.372 | 85.583 | 28.77 |
| 4344 | VAL536 | CB | 66.791 | 86.567 | 27.681 |
| 4345 | VAL536 | CG1 | 66.667 | 88.01 | 28.154 |
| 4346 | VAL536 | CG2 | 68.212 | 86.283 | 27.206 |
| 4347 | VAL536 | C | 64.946 | 85.87 | 29.211 |
| 4348 | VAL536 | O | 64.742 | 86.556 | 30.221 |
| 4349 | LEU537 | N | 63.993 | 85.238 | 28.544 |
| 4350 | LEU537 | CA | 62.574 | 85.456 | 28.847 |
| 4351 | LEU537 | CB | 61.856 | 85.784 | 27.538 |
| 4352 | LEU537 | CG | 60.352 | 85.99 | 27.721 |
| 4353 | LEU537 | CD1 | 60.056 | 87.149 | 28.666 |
| 4354 | LEU537 | CD2 | 59.668 | 86.221 | 26.379 |
| 4355 | LEU537 | C | 61.93 | 84.23 | 29.488 |
| 4356 | LEU537 | O | 61.848 | 83.156 | 28.876 |
| 4357 | LEU538 | N | 61.451 | 84.422 | 30.705 |
| 4358 | LEU538 | CA | 60.688 | 83.39 | 31.411 |
| 4359 | LEU538 | CB | 61.486 | 82.946 | 32.629 |
| 4360 | LEU538 | CG | 60.822 | 81.78 | 33.345 |
| 4361 | LEU538 | CD1 | 60.629 | 80.599 | 32.402 |
| 4362 | LEU538 | CD2 | 61.635 | 81.365 | 34.564 |
| 4363 | LEU538 | C | 59.342 | 83.972 | 31.84 |
| 4364 | LEU538 | O | 59.24 | 84.672 | 32.855 |
| 4365 | ASN539 | N | 58.323 | 83.69 | 31.052 |
| 4366 | ASN539 | CA | 57.001 | 84.285 | 31.278 |
| 4367 | ASN539 | CB | 56.517 | 84.749 | 29.911 |
| 4368 | ASN539 | CG | 55.225 | 85.551 | 29.979 |
| 4369 | ASN539 | OD1 | 54.413 | 85.485 | 29.05 |
| 4370 | ASN539 | ND2 | 55.071 | 86.332 | 31.035 |
| 4371 | ASN539 | C | 56.046 | 83.26 | 31.897 |
| 4372 | ASN539 | O | 55.503 | 82.403 | 31.196 |
| 4373 | LEU540 | N | 55.793 | 83.418 | 33.187 |
| 4374 | LEU540 | CA | 55.042 | 82.429 | 33.977 |
| 4375 | LEU540 | CB | 55.913 | 82.009 | 35.153 |
| 4376 | LEU540 | OG | 57.216 | 81.363 | 34.713 |
| 4377 | LEU540 | CD1 | 58.154 | 81.221 | 35.902 |
| 4378 | LEU540 | CD2 | 56.968 | 80.014 | 34.049 |
| 4379 | LEU540 | C | 53.742 | 82.969 | 34.569 |
| 4380 | LEU540 | O | 53.3 | 82.479 | 35.615 |
| 4381 | GLN541 | N | 53.186 | 84.012 | 33.984 |
| 4382 | GLN541 | CA | 52.046 | 84.681 | 34.62 |
| 4383 | GLN541 | CB | 51.853 | 86.02 | 33.929 |
| 4384 | GLN541 | CG | 53.138 | 86.811 | 34.118 |
| 4385 | GLN541 | CD | 53.093 | 88.175 | 33.452 |
| 4386 | GLN541 | OE1 | 53.123 | 88.278 | 32.22 |
| 4387 | GLN541 | NE2 | 53.214 | 89.196 | 34.28 |
| 4388 | GLN541 | C | 50.767 | 83.845 | 34.611 |
| 4389 | GLN541 | O | 50.437 | 83.164 | 33.637 |
| 4390 | GLY542 | N | 50.137 | 83.801 | 35.773 |
| 4391 | GLY542 | CA | 48.872 | 83.077 | 35.93 |
| 4392 | GLY542 | C | 49.081 | 81.672 | 36.486 |
| 4393 | GLY542 | O | 48.182 | 80.827 | 36.396 |
| 4394 | ASN543 | N | 50.275 | 81.42 | 36.998 |
| 4395 | ASN543 | CA | 50.601 | 80.101 | 37.551 |
| 4396 | ASN543 | CB | 51.981 | 79.679 | 37.047 |
| 4397 | ASN543 | CG | 52.046 | 79.574 | 35.524 |
| 4398 | ASN543 | OD1 | 51.046 | 79.726 | 34.816 |
| 4399 | ASN543 | ND2 | 53.233 | 79.281 | 35.033 |
| 4400 | ASN543 | C | 50.608 | 80.154 | 39.078 |
| 4401 | ASN543 | O | 50.941 | 81.192 | 39.664 |
| 4402 | PRO544 | N | 50.294 | 79.035 | 39.716 |
| 4403 | PRO544 | CA | 50.159 | 79.004 | 41.185 |
| 4404 | PRO544 | CB | 49.641 | 77.633 | 41 .497 |
| 4405 | PRO544 | CG | 49.501 | 76.832 | 40.21 |
| 4406 | PRO544 | CD | 49.912 | 77.766 | 39.086 |
| 4407 | PRO544 | C | 51.453 | 79.281 | 41.97 |
| 4408 | PRO544 | O | 51.377 | 79.897 | 43.04 |
| 4409 | LEU545 | N | 52.605 | 79.097 | 41.341 |
| 4410 | LEU545 | CA | 53.893 | 79.392 | 41.991 |
| 4411 | LEU545 | CB | 55.009 | 78.586 | 41.313 |
| 4412 | LEU545 | CG | 55.737 | 79.221 | 40.122 |
| 4413 | LEU545 | CD1 | 56.836 | 78.281 | 39.66 |
| 4414 | LEU545 | CD2 | 54.853 | 79.549 | 38.925 |
| 4415 | LEU545 | C | 54.247 | 80.885 | 42.028 |
| 4416 | LEU545 | O | 55.162 | 81.275 | 42.764 |
| 4417 | GYS546 | N | 53.418 | 81.72 | 41.417 |
| 4418 | GYS546 | CA | 53.631 | 83.167 | 41.456 |
| 4419 | GYS546 | CB | 52.957 | 83.782 | 40.239 |
| 4420 | GYS546 | SG | 53.492 | 83.109 | 38.652 |
| 4421 | GYS546 | C | 53.032 | 83.768 | 42.723 |
| 4422 | GYS546 | O | 53.393 | 84.884 | 43.113 |
| 4423 | GLN547 | N | 52.306 | 82.94 | 43.461 |
| 4424 | GLN547 | CA | 51.681 | 83.352 | 44.719 |
| 4425 | GLN547 | CB | 50.408 | 82.533 | 44.926 |
| 4426 | GLN547 | CG | 49.503 | 82.52 | 43.694 |
| 4427 | GLN547 | CD | 49.084 | 83.929 | 43.276 |
| 4428 | GLN547 | QE1 | 49.358 | 84.352 | 42.147 |
| 4429 | GLN547 | NE2 | 48.407 | 84.623 | 44.175 |
| 4430 | GLN547 | C | 52.599 | 83.171 | 45.935 |
| 4431 | GLN547 | O | 52.102 | 83.137 | 47.068 |
| 4432 | ALA548 | N | 53.891 | 82.981 | 45.702 |
| 4433 | ALA548 | CA | 54.871 | 82.835 | 46.787 |
| 4434 | ALA548 | CB | 56.191 | 82.349 | 46.201 |
| 4435 | ALA548 | C | 55.096 | 84.144 | 47.545 |
| 4436 | ALA548 | O | 54.192 | 84.98 | 47.664 |
| 4437 | VAL549 | N | 56.285 | 84.299 | 48.101 |
| 4438 | VAL549 | CA | 56.552 | 85.483 | 48.924 |
| 4439 | VAL549 | CB | 57.62 | 85.132 | 49.959 |
| 4440 | VAL549 | CG1 | 57.695 | 86.193 | 51.056 |
| 4441 | VAL549 | CG2 | 57.324 | 83.774 | 50.585 |
| 4442 | VAL549 | C | 57.021 | 86.625 | 48.026 |
| 4443 | VAL549 | O | 56.219 | 87.441 | 47.553 |
| 4444 | GLYS50 | N | 58.295 | 86.588 | 47.688 |
| 4445 | GLYS50 | CA | 58.874 | 87.57 | 46.777 |
| 4446 | GLYS50 | C | 59.369 | 86.779 | 45.584 |
| 4447 | GLYS50 | O | 60.574 | 86.724 | 45.3 |
| 4448 | 1LEA551 | N | 58.414 | 86.324 | 44.79 |
| 4449 | 1LEA551 | CA | 58.697 | 85.317 | 43.762 |
| 4450 | 1LEA551 | OB | 57.356 | 84.768 | 43.272 |
| 4451 | 1LEA551 | CG2 | 56.499 | 85.845 | 42.614 |
| 4452 | 1LEA551 | CG1 | 57.548 | 83.584 | 42.336 |
| 4453 | 1LEA551 | CD1 | 58.227 | 82.428 | 43.062 |
| 4454 | 1LEA551 | C | 59.561 | 85.84 | 42.607 |
| 4455 | 1LEA551 | O | 60.407 | 85.077 | 42.124 |
| 4456 | LEU552 | N | 59.624 | 87.152 | 42.437 |
| 4457 | LEU552 | CA | 60.486 | 87.731 | 41.407 |
| 4458 | LEU552 | CB | 60.132 | 89.204 | 41.253 |
| 4459 | LEU552 | CG | 58.727 | 89.386 | 40.693 |
| 4460 | LEU552 | CD1 | 58.28 | 90.839 | 40.788 |
| 4461 | LEU552 | CD2 | 58.645 | 88.888 | 39.256 |
| 4462 | LEU552 | C | 61.956 | 87.596 | 41.787 |
| 4463 | LEU552 | O | 62.713 | 86.961 | 41.042 |
| 4464 | GLU553 | N | 62.293 | 87.92 | 43.027 |
| 4465 | GLU553 | CA | 63.704 | 87.837 | 43.416 |
| 4466 | GLU553 | CB | 64.09 | 88.895 | 44.461 |
| 4467 | GLU553 | CG | 63.89 | 88.51 | 45.931 |
| 4468 | GLU553 | CD | 62.455 | 88.723 | 46.401 |
| 4469 | GLU553 | OE1 | 61.698 | 89.338 | 45.658 |
| 4470 | GLU553 | OE2 | 62.11 | 88.175 | 47.438 |
| 4471 | GLU553 | C | 64.075 | 86.434 | 43.886 |
| 4472 | GLU553 | O | 65.247 | 86.066 | 43.762 |
| 4473 | GLN554 | N | 63.092 | 85.594 | 44.17 |
| 4474 | GLN554 | CA | 63.409 | 84.214 | 44.527 |
| 4475 | GLN554 | CB | 62.222 | 83.59 | 45.254 |
| 4476 | GLN554 | CG | 61.966 | 84.309 | 46.577 |
| 4477 | GLN554 | CD | 60.8 | 83.682 | 47.341 |
| 4478 | GLN554 | OE1 | 59.625 | 84.018 | 47.13 |
| 4479 | GLN554 | NE2 | 61.148 | 82.835 | 48.292 |
| 4480 | GLN554 | C | 63.754 | 83.426 | 43.27 |
| 4481 | GLN554 | O | 64.827 | 82.809 | 43.218 |
| 4482 | LEU555 | N | 63.059 | 83.73 | 42.186 |
| 4483 | LEU555 | CA | 63.372 | 83.068 | 40.919 |
| 4484 | LEU555 | CB | 62.16 | 83.111 | 40.004 |
| 4485 | LEU555 | CG | 61 .027 | 82.277 | 40.578 |
| 4486 | LEU555 | CD1 | 59.804 | 82.344 | 39.673 |
| 4487 | LEU555 | CD2 | 61.471 | 80.833 | 40.789 |
| 4488 | LEU555 | C | 64.566 | 83.701 | 40.223 |
| 4489 | LEU555 | O | 65.324 | 82.973 | 39.577 |
| 4490 | ALA556 | N | 64.891 | 84.935 | 40.568 |
| 4491 | ALA556 | CA | 66.113 | 85.545 | 40.034 |
| 4492 | ALA556 | CB | 66.033 | 87.056 | 40.227 |
| 4493 | ALA556 | C | 67.367 | 85.009 | 40.727 |
| 4494 | ALA556 | O | 68.398 | 84.832 | 40.067 |
| 4495 | GLU557 | N | 67.206 | 84.527 | 41.951 |
| 4496 | GLU557 | CA | 68.324 | 83.922 | 42.682 |
| 4497 | GLU557 | CB | 68.039 | 84.044 | 44.174 |
| 4498 | GLU557 | CG | 68.06 | 85.499 | 44.622 |
| 4499 | GLU557 | CD | 67.376 | 85.643 | 45.978 |
| 4500 | GLU557 | OE1 | 66.545 | 84.801 | 46.292 |
| 4501 | GLU557 | OE2 | 67.612 | 86.652 | 46.628 |
| 4502 | GLU557 | C | 68.512 | 82.45 | 42.327 |
| 4503 | GLU557 | O | 69.584 | 81.888 | 42.577 |
| 4504 | LEU558 | N | 67.506 | 81.849 | 41.713 |
| 4505 | LEU558 | CA | 67.639 | 80.461 | 41.267 |
| 4506 | LEU558 | CB | 66.294 | 79.765 | 41.445 |
| 4507 | LEU558 | CG | 65.833 | 79.752 | 42.898 |
| 4508 | LEU558 | CD1 | 64.424 | 79.181 | 43.009 |
| 4509 | LEU558 | CD2 | 66.803 | 78.979 | 43.786 |
| 4510 | LEU558 | C | 68.026 | 80.392 | 39.796 |
| 4511 | LEU558 | O | 68.67 | 79.428 | 39.357 |
| 4512 | LEU559 | N | 67.62 | 81.404 | 39.046 |
| 4513 | LEU559 | CA | 67.883 | 81.457 | 37.601 |
| 4514 | LEU559 | CB | 66.537 | 81.352 | 36.881 |
| 4515 | LEU559 | CG | 65.673 | 80.196 | 37.381 |
| 4516 | LEU559 | CD1 | 64.234 | 80.329 | 36.9 |
| 4517 | LEU559 | CD2 | 66.249 | 78.843 | 36.99 |
| 4518 | LEU559 | C | 68.505 | 82.796 | 37.192 |
| 4519 | LEU559 | O | 67.874 | 83.526 | 36.417 |
| 4520 | PRO560 | N | 69.796 | 82.973 | 37.449 |
| 4521 | PRO560 | CA | 70.418 | 84.31 | 37.391 |
| 4522 | PRO560 | CB | 71.682 | 84.176 | 38.183 |
| 4523 | PRO560 | CG | 71.94 | 82.708 | 38.477 |
| 4524 | PRO560 | CD | 70.727 | 81.957 | 37.96 |
| 4525 | PRO560 | C | 70.742 | 84.829 | 35.98 |
| 4526 | PRO560 | O | 71 .278 | 85.933 | 35.844 |
| 4527 | SER561 | N | 70.454 | 84.047 | 34.951 |
| 4528 | SER561 | CA | 70.725 | 84.481 | 33.58 |
| 4529 | SER561 | CB | 71.287 | 83.3 | 32.803 |
| 4530 | SER561 | OG | 72.471 | 82.879 | 33.466 |
| 4531 | SER561 | C | 69.459 | 84.993 | 32.898 |
| 4532 | SER561 | O | 69.534 | 85.686 | 31.875 |
| 4533 | VAL562 | N | 68.317 | 84.708 | 33.504 |
| 4534 | VAL562 | CA | 67.046 | 85.165 | 32.947 |
| 4535 | VAL562 | CB | 65.933 | 84.265 | 33.467 |
| 4536 | VAL562 | CG1 | 64.601 | 84.631 | 32.828 |
| 4537 | VAL562 | CG2 | 66.256 | 82.8 | 33.2 |
| 4538 | VAL562 | C | 66.817 | 86.608 | 33.373 |
| 4539 | VAL562 | O | 66.612 | 86.906 | 34.556 |
| 4540 | SER563 | N | 66.813 | 87.493 | 32.392 |
| 4541 | SER563 | CA | 66.731 | 88.924 | 32.682 |
| 4542 | SER563 | CB | 67.49 | 89.663 | 31.589 |
| 4543 | SER563 | OG | 68.805 | 89.121 | 31.552 |
| 4544 | SER563 | C | 65.286 | 89.405 | 32.732 |
| 4545 | SER563 | O | 64.981 | 90.429 | 33.354 |
| 4546 | SER564 | N | 64.397 | 88.61 | 32.167 |
| 4547 | SER564 | CA | 62.974 | 88.925 | 32.211 |
| 4548 | SER564 | CB | 62.488 | 89.148 | 30.786 |
| 4549 | SER564 | OG | 61.107 | 89.467 | 30.852 |
| 4550 | SER564 | C | 62.192 | 87.79 | 32.857 |
| 4551 | SER564 | O | 61.62 | 86.937 | 32.162 |
| 4552 | VAL565 | N | 62.215 | 87.762 | 34.179 |
| 4553 | VAL565 | CA | 61.421 | 86.784 | 34.934 |
| 4554 | VAL565 | CB | 62.125 | 86.463 | 36.251 |
| 4555 | VAL565 | CG1 | 61.412 | 85.333 | 36.987 |
| 4556 | VAL565 | CG2 | 63.586 | 86.092 | 36.026 |
| 4557 | VAL565 | C | 60.043 | 87.376 | 35.222 |
| 4558 | VAL565 | O | 59.812 | 87.998 | 36.266 |
| 4559 | LEU566 | N | 59.122 | 87.141 | 34.308 |
| 4560 | LEU566 | CA | 57.798 | 87.75 | 34.408 |
| 4561 | LEU566 | CB | 57.323 | 88.157 | 33.021 |
| 4562 | LEU566 | CG | 58.212 | 89.226 | 32.401 |
| 4563 | LEU566 | CD1 | 57.768 | 89.527 | 30.975 |
| 4564 | LEU566 | CD2 | 58.213 | 90.498 | 33.244 |
| 4565 | LEU566 | C | 56.795 | 86.785 | 35.014 |
| 4566 | LEU566 | O | 56.117 | 86.039 | 34.295 |
| 4567 | THR567 | N | 56.687 | 86.837 | 36.329 |
| 4568 | THR567 | CA | 55.709 | 86.012 | 37.045 |
| 4569 | THR567 | CB | 56.393 | 85.328 | 38.222 |
| 4570 | THR567 | OG1 | 56.733 | 86.313 | 39.186 |
| 4571 | THR567 | CG2 | 57.661 | 84.604 | 37.791 |
| 4572 | THR567 | C | 54.561 | 86.88 | 37.553 |
| 4573 | THR567 | O | 53.882 | 86.441 | 38.47 |
| 4574 | THR567 | OXT | 54.277 | 87.875 | 36.901 |

TABLE 12

| | Residue/ | | | | |
|---|---|---|---|---|---|
| Atom | Residue | Atom | X | Y | Z |
| No. | Position | Type | Coord. | Coord. | Coord. |
| 1 | MET1 | N | 25.639 | 32.902 | 36.49 |
| 2 | MET1 | CA | 26.981 | 32.307 | 36.329 |
| 3 | MET1 | CB | 27.631 | 32.812 | 35.043 |
| 4 | MET1 | CG | 27.797 | 34.332 | 35.059 |
| 5 | MET1 | SD | 28.559 | 35.081 | 33.602 |
| 6 | MET1 | CE | 27.379 | 34.546 | 32.344 |
| 7 | MET1 | C | 27.872 | 32.687 | 37.507 |
| 8 | MET1 | O | 29.046 | 32.298 | 37.586 |
| 9 | GLY2 | N | 27.289 | 33.443 | 38.422 |
| 10 | GLY2 | CA | 28.052 | 34.024 | 39.53 |
| 11 | GLY2 | C | 28.827 | 35.244 | 39.024 |
| 12 | GLY2 | O | 28.333 | 36.377 | 39.024 |
| 13 | THR3 | N | 30.035 | 34.979 | 38.567 |
| 14 | THR3 | CA | 30.902 | 35.999 | 37.96 |
| 15 | THR3 | CB | 31.984 | 36.436 | 38.95 |
| 16 | THR3 | OG1 | 32.457 | 35.292 | 39.638 |
| 17 | THR3 | CG2 | 31.428 | 37.396 | 39.999 |
| 18 | THR3 | C | 31.522 | 35.604 | 36.595 |
| 19 | THR3 | O | 31.389 | 36.424 | 35.673 |
| 20 | PRO4 | N | 32.202 | 34.465 | 36.43 |
| 21 | PRO4 | CA | 32.942 | 34.247 | 35.182 |
| 22 | PRO4 | CB | 33.867 | 33.101 | 35.448 |
| 23 | PRO4 | CG | 33.544 | 32.483 | 36.794 |
| 24 | PRO4 | CD | 32.439 | 33.345 | 37.367 |
| 25 | PRO4 | C | 32.047 | 33.916 | 33.997 |
| 26 | PRO4 | O | 31.125 | 33.099 | 34.091 |
| 27 | GLN5 | N | 32.347 | 34.573 | 32.891 |
| 28 | GLN5 | CA | 31.735 | 34.265 | 31.6 |
| 29 | GLN5 | CB | 31.439 | 35.59 | 30.908 |
| 30 | GLN5 | CG | 30.341 | 35.538 | 29.846 |
| 31 | GLN5 | CD | 30.807 | 36.449 | 28.72 |
| 32 | GLN5 | OE1 | 32.01 | 36.478 | 28.417 |
| 33 | GLN5 | NE2 | 29.886 | 37.203 | 28.15 |
| 34 | GLN5 | C | 32.772 | 33.455 | 30.818 |
| 35 | GLN5 | O | 33.19 | 32.381 | 31.264 |
| 36 | LYS6 | N | 33.151 | 33.954 | 29.655 |
| 37 | LYS6 | CA | 34.263 | 33.396 | 28.891 |
| 38 | LYS6 | CB | 33.766 | 32.99 | 27.509 |
| 39 | LYS6 | CG | 32.679 | 31.926 | 27.595 |
| 40 | LYS6 | CD | 32.192 | 31.506 | 26.214 |
| 41 | LYS6 | CE | 31.128 | 30.416 | 26.314 |
| 42 | LYS6 | NZ | 30.67 | 30.005 | 24.975 |
| 43 | LYS6 | C | 35.314 | 34.484 | 28.772 |
| 44 | LYS6 | O | 36.507 | 34.266 | 29.012 |
| 45 | ASP7 | N | 34.817 | 35.691 | 28.567 |
| 46 | ASP7 | CA | 35.672 | 36.867 | 28.512 |
| 47 | ASP7 | CB | 35.499 | 37.527 | 27.149 |
| 48 | ASP7 | CG | 36.269 | 38.843 | 27.06 |
| 49 | ASP7 | OD1 | 37.47 | 38.802 | 26.825 |
| 50 | ASP7 | OD2 | 35.622 | 39.878 | 27.147 |
| 51 | ASP7 | C | 35.315 | 37.833 | 29.633 |
| 52 | ASP7 | O | 36.055 | 37.941 | 30.614 |
| 53 | VAL8 | N | 34.128 | 38.409 | 29.58 |
| 54 | VAL8 | CA | 33.82 | 39.486 | 30.528 |
| 55 | VAL8 | CB | 33.198 | 40.657 | 29.767 |
| 56 | VAL8 | CG1 | 32.082 | 40.208 | 28.833 |
| 57 | VAL8 | CG2 | 32.73 | 41.768 | 30.701 |
| 58 | VAL8 | C | 32.943 | 39.027 | 31.689 |
| 59 | VAL8 | O | 31.8 | 38.589 | 31.516 |
| 60 | ILE9 | N | 33.55 | 39.055 | 32.863 |
| 61 | ILE9 | CA | 32.847 | 38.788 | 34.123 |
| 62 | ILE9 | CB | 33.9 | 38.825 | 35.231 |
| 63 | ILE9 | CG2 | 33.334 | 39.118 | 36.619 |
| 64 | ILE9 | CG1 | 34.691 | 37.53 | 35.248 |
| 65 | ILE9 | CD1 | 35.65 | 37.525 | 36.426 |
| 66 | ILE9 | C | 31.754 | 39.821 | 34.383 |
| 67 | ILE9 | O | 31.93 | 41.012 | 34.101 |
| 68 | ILE10 | N | 30.595 | 39.347 | 34.81 |
| 69 | ILE10 | CA | 29.527 | 40.263 | 35.21 |
| 70 | ILE10 | CB | 28.201 | 39.512 | 35.158 |
| 71 | ILE10 | CG2 | 27.057 | 40.376 | 35.676 |
| 72 | ILE10 | CG1 | 27.914 | 39.042 | 33.738 |
| 73 | ILE10 | CD1 | 26.564 | 38.342 | 33.659 |
| 74 | ILE10 | C | 29.798 | 40.793 | 36.619 |
| 75 | ILE10 | O | 29.82 | 40.033 | 37.596 |
| 76 | LYS11 | N | 30.081 | 42.083 | 36.701 |
| 77 | LYS11 | CA | 30.324 | 42.717 | 38.001 |
| 78 | LYS11 | CB | 30.964 | 44.083 | 37.781 |
| 79 | LYS11 | CG | 31.214 | 44.785 | 39.113 |
| 80 | LYS11 | CD | 31.653 | 46.231 | 38.918 |
| 81 | LYS11 | CE | 31.783 | 46.946 | 40.258 |
| 82 | LYS11 | NZ | 32.095 | 48.37 | 40.067 |
| 83 | LYS11 | C | 29.023 | 42.892 | 38.782 |
| 84 | LYS11 | O | 28.163 | 43.708 | 38.433 |
| 85 | SER12 | N | 28.886 | 42.09 | 39.823 |
| 86 | SER12 | CA | 27.739 | 42.194 | 40.727 |
| 87 | SER12 | CB | 27.424 | 40.805 | 41.266 |
| 88 | SER12 | OG | 27.184 | 39.959 | 40.148 |
| 89 | SER12 | C | 28.059 | 43.148 | 41.874 |
| 90 | SER12 | O | 29.087 | 43.836 | 41.853 |
| 91 | ASP13 | N | 27.158 | 43.225 | 42.841 |
| 92 | ASP13 | CA | 27.386 | 44.063 | 44.033 |
| 93 | ASP13 | CB | 26.047 | 44.467 | 44.658 |
| 94 | ASP13 | CG | 25.103 | 43.279 | 44.868 |
| 95 | ASP13 | OD1 | 24.338 | 42.997 | 43.956 |
| 96 | ASP13 | OD2 | 25.103 | 42.732 | 45.961 |
| 97 | ASP13 | C | 28.3 | 43.365 | 45.048 |
| 98 | ASP13 | O | 27.861 | 42.738 | 46.017 |
| 99 | ALA14 | N | 29.588 | 43.499 | 44.795 |
| 100 | ALA14 | CA | 30.628 | 42.871 | 45.611 |
| 101 | ALA14 | CB | 31.578 | 42.199 | 44.623 |
| 102 | ALA14 | C | 31.33 | 43.935 | 46.463 |
| 103 | ALA14 | O | 30.992 | 45.117 | 46.327 |
| 104 | PRO15 | N | 32.204 | 43.534 | 47.382 |
| 105 | PRO15 | CA | 32.877 | 44.504 | 48.259 |
| 106 | PRO15 | CB | 33.846 | 43.709 | 49.078 |
| 107 | PRO15 | CG | 33.671 | 42.234 | 48.764 |
| 108 | PRO15 | CD | 32.579 | 42.152 | 47.712 |
| 109 | PRO15 | C | 33.585 | 45.613 | 47.486 |
| 110 | PRO15 | O | 34.004 | 45.445 | 46.334 |
| 111 | ASP16 | N | 33.502 | 46.806 | 48.045 |
| 112 | ASP16 | CA | 34.174 | 47.965 | 47.447 |
| 113 | ASP16 | CB | 33.155 | 48.889 | 46.77 |
| 114 | ASP16 | CG | 31.992 | 49.3 | 47.678 |
| 115 | ASP16 | OD1 | 30.888 | 49.394 | 47.163 |
| 116 | ASP16 | OD2 | 32.21 | 49.499 | 48.867 |
| 117 | ASP16 | C | 35.017 | 48.712 | 48.477 |
| 118 | ASP16 | O | 35.681 | 49.707 | 48.166 |
| 119 | THR17 | N | 34.967 | 48.235 | 49.705 |
| 120 | THR17 | CA | 35.724 | 48.867 | 50.782 |
| 121 | THR17 | CB | 34.769 | 49.09 | 51.948 |
| 122 | THR17 | OG1 | 33.657 | 49.824 | 51.451 |
| 123 | THR17 | CG2 | 35.409 | 49.887 | 53.08 |
| 124 | THR17 | C | 36.867 | 47.951 | 51.187 |
| 125 | THR17 | O | 36.627 | 46.785 | 51.507 |
| 126 | LEU18 | N | 38.082 | 48.474 | 51.107 |
| 127 | LEU18 | CA | 39.308 | 47.718 | 51.418 |
| 128 | LEU18 | CB | 40.471 | 48.709 | 51.364 |
| 129 | LEU18 | CG | 41.82 | 48.074 | 51.697 |
| 130 | LEU18 | CD1 | 42.217 | 47.033 | 50.659 |
| 131 | LEU18 | CD2 | 42.904 | 49.139 | 51.812 |
| 132 | LEU18 | C | 39.263 | 47.064 | 52.8 |
| 133 | LEU18 | O | 39.369 | 47.739 | 53.833 |
| 134 | LEU19 | N | 39.174 | 45.743 | 52.803 |
| 135 | LEU19 | CA | 39.182 | 44.964 | 54.049 |
| 136 | LEU19 | CB | 38.427 | 43.665 | 53.793 |
| 137 | LEU19 | CG | 37.009 | 43.921 | 53.3 |
| 138 | LEU19 | CD1 | 36.368 | 42.634 | 52.8 |
| 139 | LEU19 | CD2 | 36.151 | 44.583 | 54.373 |
| 140 | LEU19 | C | 40.605 | 44.622 | 54.476 |
| 141 | LEU19 | O | 40.918 | 43.444 | 54.689 |
| 142 | LEU20 | N | 41.37 | 45.643 | 54.827 |
| 143 | LEU20 | CA | 42.814 | 45.489 | 55.054 |
| 144 | LEU20 | CB | 43.401 | 46.886 | 55.227 |
| 145 | LEU20 | CG | 44.913 | 46.889 | 55.046 |
| 146 | LEU20 | CD1 | 45.263 | 46.478 | 53.621 |
| 147 | LEU20 | CD2 | 45.497 | 48.263 | 55.354 |
| 148 | LEU20 | C | 43.14 | 44.648 | 56.289 |
| 149 | LEU20 | O | 44.006 | 43.765 | 56.215 |
| 150 | GLU21 | N | 42.277 | 44.717 | 57.291 |
| 151 | GLU21 | CA | 42.482 | 43.909 | 58.495 |
| 152 | GLU21 | CB | 41.594 | 44.441 | 59.612 |
| 153 | GLU21 | CG | 41.766 | 43.635 | 60.897 |
| 154 | GLU21 | CD | 40.796 | 44.15 | 61.954 |
| 155 | GLU21 | OE1 | 40.278 | 45.239 | 61.746 |
| 156 | GLU21 | OE2 | 40.515 | 43.417 | 62.891 |
| 157 | GLU21 | C | 42.135 | 42.448 | 58.242 |
| 158 | GLU21 | O | 42.942 | 41.581 | 58.595 |
| 159 | LYS22 | N | 41.187 | 42.211 | 57.351 |
| 160 | LYS22 | CA | 40.761 | 40.843 | 57.074 |
| 161 | LYS22 | CB | 39.388 | 40.881 | 56.418 |
| 162 | LYS22 | CG | 38.319 | 41.477 | 57.323 |
| 163 | LYS22 | CD | 36.963 | 41.438 | 56.628 |
| 164 | LYS22 | CE | 35.864 | 42.038 | 57.495 |
| 165 | LYS22 | NZ | 34.572 | 42.015 | 56.79 |
| 166 | LYS22 | C | 41.738 | 40.149 | 56.135 |
| 167 | LYS22 | O | 41.974 | 38.943 | 56.286 |
| 168 | HIS23 | N | 42.452 | 40.925 | 55.336 |
| 169 | HIS23 | CA | 43.46 | 40.338 | 54.452 |
| 170 | HIS23 | CB | 43.885 | 41.35 | 53.393 |
| 171 | HIS23 | CG | 42.774 | 41.931 | 52.537 |
| 172 | HIS23 | ND1 | 41.683 | 41.303 | 52.059 |
| 173 | HIS23 | CE1 | 40.942 | 42.171 | 51.35 |
| 174 | HIS23 | NE2 | 41.58 | 43.362 | 51.366 |
| 175 | HIS23 | CD2 | 42.718 | 43.226 | 52.087 |
| 176 | HIS23 | C | 44.684 | 39.943 | 55.263 |
| 177 | HIS23 | O | 45.124 | 38.79 | 55.164 |
| 178 | ALA24 | N | 45.005 | 40.754 | 56.261 |
| 179 | ALA24 | CA | 46.152 | 40.463 | 57.125 |
| 180 | ALA24 | CB | 46.493 | 41.723 | 57.905 |
| 181 | ALA24 | C | 45.859 | 39.325 | 58.095 |
| 182 | ALA24 | O | 46.68 | 38.403 | 58.22 |
| 183 | ASP25 | N | 44.609 | 39.246 | 58.528 |
| 184 | ASP25 | CA | 44.166 | 38.149 | 59.391 |
| 185 | ASP25 | CB | 42.719 | 38.383 | 59.824 |
| 186 | ASP25 | CG | 42.57 | 39.629 | 60.696 |
| 187 | ASP25 | OD1 | 43.493 | 39.926 | 61.442 |
| 188 | ASP25 | OD2 | 41.501 | 40.226 | 60.65 |
| 189 | ASP25 | C | 44.232 | 36.824 | 58.647 |
| 190 | ASP25 | O | 44.936 | 35.918 | 59.11 |
| 191 | TYR26 | N | 43.786 | 36.827 | 57.4 |
| 192 | TYR26 | CA | 43.784 | 35.606 | 56.592 |
| 193 | TYR26 | CB | 43.004 | 35.88 | 55.308 |
| 194 | TYR26 | CG | 43.125 | 34.783 | 54.252 |
| 195 | TYR26 | CD1 | 42.393 | 33.61 | 54.377 |
| 196 | TYR26 | CE1 | 42.521 | 32.608 | 53.423 |
| 197 | TYR26 | CZ | 43.378 | 32.786 | 52.346 |
| 198 | TYR26 | OH | 43.577 | 31.757 | 51.453 |
| 199 | TYR26 | CE2 | 44.098 | 33.964 | 52.208 |
| 200 | TYR26 | CD2 | 43.969 | 34.964 | 53.162 |
| 201 | TYR26 | C | 45.189 | 35.13 | 56.236 |
| 202 | TYR26 | O | 45.471 | 33.939 | 56.398 |
| 203 | ILE27 | N | 46.108 | 36.049 | 55.988 |
| 204 | ILE27 | CA | 47.455 | 35.628 | 55.591 |
| 205 | ILE27 | CB | 48.165 | 36.79 | 54.905 |
| 206 | ILE27 | CG2 | 49.602 | 36.408 | 54.568 |
| 207 | ILE27 | CG1 | 47.432 | 37.204 | 53.636 |
| 208 | ILE27 | CD1 | 47.451 | 36.085 | 52.601 |
| 209 | ILE27 | C | 48.282 | 35.137 | 56.777 |
| 210 | ILE27 | O | 48.914 | 34.077 | 56.667 |
| 211 | ALA28 | N | 48.064 | 35.71 | 57.95 |
| 212 | ALA28 | CA | 48.816 | 35.25 | 59.123 |
| 213 | ALA28 | CB | 48.823 | 36.354 | 60.171 |
| 214 | ALA28 | C | 48.21 | 33.976 | 59.709 |
| 215 | ALA28 | O | 48.942 | 33.08 | 60.15 |
| 216 | SER29 | N | 46.918 | 33.799 | 59.487 |
| 217 | SER29 | CA | 46.236 | 32.583 | 59.93 |
| 218 | SER29 | CB | 44.776 | 32.899 | 60.225 |
| 219 | SER29 | OG | 44.145 | 33.2 | 58.988 |
| 220 | SER29 | C | 46.284 | 31.469 | 58.889 |
| 221 | SER29 | O | 45.878 | 30.347 | 59.206 |
| 222 | TYR30 | N | 46.922 | 31.697 | 57.75 |
| 223 | TYR30 | CA | 46.937 | 30.681 | 56.693 |
| 224 | TYR30 | CB | 47.36 | 31.343 | 55.386 |
| 225 | TYR30 | CG | 47.285 | 30.42 | 54.174 |
| 226 | TYR30 | CD1 | 46.057 | 30.175 | 53.572 |
| 227 | TYR30 | CE1 | 45.98 | 29.33 | 52.472 |
| 228 | TYR30 | CZ | 47.133 | 28.734 | 51.98 |
| 229 | TYR30 | OH | 47.06 | 27.902 | 50.885 |
| 230 | TYR30 | CE2 | 48.361 | 28.977 | 52.578 |
| 231 | TYR30 | CD2 | 48.436 | 29.823 | 53.678 |
| 232 | TYR30 | C | 47.877 | 29.528 | 57.031 |
| 233 | TYR30 | O | 47.524 | 28.367 | 56.788 |
| 234 | GLY31 | N | 48.872 | 29.813 | 57.856 |
| 235 | GLY31 | CA | 49.777 | 28.765 | 58.34 |
| 236 | GLY31 | C | 49.276 | 28.164 | 59.654 |
| 237 | GLY31 | O | 49.84 | 27.189 | 60.161 |
| 238 | SER32 | N | 48.206 | 28.741 | 60.176 |
| 239 | SER32 | CA | 47.602 | 28.29 | 61.426 |
| 240 | SER32 | CB | 47.293 | 29.527 | 62.261 |
| 241 | SER32 | OG | 48.479 | 30.309 | 62.323 |
| 242 | SER32 | C | 46.309 | 27.514 | 61.171 |
| 243 | SER32 | O | 45.659 | 27.072 | 62.127 |
| 244 | LYS33 | N | 45.923 | 27.397 | 59.909 |
| 245 | LYS33 | CA | 44.703 | 26.669 | 59.544 |
| 246 | LYS33 | CB | 44.376 | 26.939 | 58.078 |
| 247 | LYS33 | CG | 43.771 | 28.319 | 57.858 |
| 248 | LYS33 | CD | 43.464 | 28.547 | 56.382 |
| 249 | LYS33 | CE | 42.648 | 29.817 | 56.167 |
| 250 | LYS33 | NZ | 43.346 | 30.996 | 56.697 |
| 251 | LYS33 | C | 44.854 | 25.167 | 59.739 |
| 252 | LYS33 | O | 44.734 | 24.647 | 60.855 |
| 253 | LYS34 | N | 44.978 | 24.471 | 58.624 |
| 254 | LYS34 | CA | 45.114 | 23.015 | 58.66 |
| 255 | LYS34 | CB | 44.185 | 22.392 | 57.628 |
| 256 | LYS34 | CG | 42.726 | 22.508 | 58.048 |
| 257 | LYS34 | CD | 41.807 | 21.82 | 57.046 |
| 258 | LYS34 | CE | 42.171 | 20.348 | 56.869 |
| 259 | LYS34 | NZ | 42.041 | 19.601 | 58.131 |
| 260 | LYS34 | C | 46.54 | 22.574 | 58.383 |
| 261 | LYS34 | O | 47.288 | 23.228 | 57.651 |
| 262 | ASP35 | N | 46.871 | 21.413 | 58.923 |
| 263 | ASP35 | CA | 48.185 | 20.803 | 58.688 |
| 264 | ASP35 | CB | 48.464 | 19.864 | 59.862 |
| 265 | ASP35 | CG | 49.801 | 19.141 | 59.705 |
| 266 | ASP35 | OD1 | 49.784 | 18.021 | 59.214 |
| 267 | ASP35 | OD2 | 50.8 | 19.695 | 60.138 |
| 268 | ASP35 | C | 48.208 | 20.019 | 57.373 |
| 269 | ASP35 | O | 49.267 | 19.817 | 56.772 |
| 270 | ASP36 | N | 47.031 | 19.689 | 56.869 |
| 271 | ASP36 | CA | 46.935 | 18.925 | 55.624 |
| 272 | ASP36 | CB | 45.951 | 17.772 | 55.812 |
| 273 | ASP36 | CG | 46.412 | 16.824 | 56.919 |
| 274 | ASP36 | OD1 | 46.944 | 15.775 | 56.587 |
| 275 | ASP36 | OD2 | 46.1 | 17.11 | 58.07 |
| 276 | ASP36 | C | 46.454 | 19.827 | 54.497 |
| 277 | ASP36 | O | 47.258 | 20.556 | 53.896 |
| 278 | TYR37 | N | 45.136 | 19.855 | 54.334 |
| 279 | TYR37 | CA | 44.437 | 20.613 | 53.276 |
| 280 | TYR37 | CB | 44.204 | 22.028 | 53.813 |
| 281 | TYR37 | CG | 42.966 | 22.75 | 53.276 |
| 282 | TYR37 | CD1 | 41.867 | 22.018 | 52.844 |
| 283 | TYR37 | CE1 | 40.747 | 22.674 | 52.35 |
| 284 | TYR37 | CZ | 40.731 | 24.061 | 52.291 |
| 285 | TYR37 | OH | 39.63 | 24.711 | 51.778 |
| 286 | TYR37 | CE2 | 41.826 | 24.796 | 52.725 |
| 287 | TYR37 | CD2 | 42.946 | 24.138 | 53.219 |
| 288 | TYR37 | C | 45.252 | 20.626 | 51.978 |
| 289 | TYR37 | O | 45.886 | 19.627 | 51.618 |
| 290 | GLU38 | N | 45.291 | 21.767 | 51.315 |
| 291 | GLU38 | CA | 46.163 | 21.911 | 50.151 |
| 292 | GLU38 | CB | 45.486 | 22.723 | 49.04 |
| 293 | GLU38 | CG | 44.802 | 24.021 | 49.477 |
| 294 | GLU38 | CD | 45.791 | 25.093 | 49.927 |
| 295 | GLU38 | OE1 | 46.327 | 25.787 | 49.078 |
| 296 | GLU38 | OE2 | 45.998 | 25.17 | 51.132 |
| 297 | GLU38 | C | 47.53 | 22.48 | 50.53 |
| 298 | GLU38 | O | 48.36 | 22.669 | 49.64 |
| 299 | TYR39 | N | 47.822 | 22.588 | 51.817 |
| 300 | TYR39 | CA | 49.075 | 23.206 | 52.252 |
| 301 | TYR39 | CB | 48.932 | 23.577 | 53.726 |
| 302 | TYR39 | CG | 50.053 | 24.449 | 54.287 |
| 303 | TYR39 | CD1 | 49.914 | 25.831 | 54.281 |
| 304 | TYR39 | CE1 | 50.927 | 26.634 | 54.788 |
| 305 | TYR39 | CZ | 52.075 | 26.051 | 55.305 |
| 306 | TYR39 | OH | 53.087 | 26.847 | 55.795 |
| 307 | TYR39 | CE2 | 52.214 | 24.669 | 55.321 |
| 308 | TYR39 | CD2 | 51.2 | 23.868 | 54.815 |
| 309 | TYR39 | C | 50.216 | 22.213 | 52.064 |
| 310 | TYR39 | O | 51.216 | 22.548 | 51.42 |
| 311 | CYS40 | N | 49.895 | 20.947 | 52.274 |
| 312 | CYS40 | CA | 50.872 | 19.881 | 52.031 |
| 313 | CYS40 | CB | 50.383 | 18.629 | 52.751 |
| 314 | CYS40 | SG | 51.432 | 17.165 | 52.592 |
| 315 | CYS40 | C | 51.036 | 19.587 | 50.536 |
| 316 | CYS40 | O | 52.134 | 19.226 | 50.095 |
| 317 | MET41 | N | 50.052 | 19.992 | 49.748 |
| 318 | MET41 | CA | 50.1 | 19.771 | 48.303 |
| 319 | MET41 | CB | 48.665 | 19.663 | 47.806 |
| 320 | MET41 | CG | 47.902 | 18.589 | 48.571 |
| 321 | MET41 | SD | 46.164 | 18.402 | 48.116 |
| 322 | MET41 | CE | 46.376 | 18.033 | 46.359 |
| 323 | MET41 | C | 50.798 | 20.93 | 47.598 |
| 324 | MET41 | O | 51.459 | 20.729 | 46.575 |
| 325 | SER42 | N | 50.81 | 22.078 | 48.255 |
| 326 | SER42 | CA | 51.479 | 23.272 | 47.74 |
| 327 | SER42 | CB | 50.635 | 24.494 | 48.076 |
| 328 | SER42 | OG | 49.349 | 24.311 | 47.497 |
| 329 | SER42 | C | 52.884 | 23.421 | 48.317 |
| 330 | SER42 | O | 53.572 | 24.41 | 48.039 |
| 331 | GLU43 | N | 53.375 | 22.364 | 48.946 |
| 332 | GLU43 | CA | 54.723 | 22.368 | 49.52 |
| 333 | GLU43 | CB | 54.838 | 21.152 | 50.436 |
| 334 | GLU43 | CG | 56.117 | 21.155 | 51.266 |
| 335 | GLU43 | CD | 56.092 | 22.287 | 52.293 |
| 336 | GLU43 | OE1 | 57.164 | 22.666 | 52.744 |
| 337 | GLU43 | OE2 | 55.002 | 22.604 | 52.747 |
| 338 | GLU43 | C | 55.817 | 22.318 | 48.444 |
| 339 | GLU43 | O | 56.936 | 22.767 | 48.708 |
| 340 | TYR44 | N | 55.443 | 22.024 | 47.205 |
| 341 | TYR44 | CA | 56.393 | 22.079 | 46.084 |
| 342 | TYR44 | CB | 55.927 | 21.153 | 44.957 |
| 343 | TYR44 | CG | 54.778 | 21.667 | 44.085 |
| 344 | TYR44 | CD1 | 55.054 | 22.248 | 42.852 |
| 345 | TYR44 | CE1 | 54.018 | 22.717 | 42.055 |
| 346 | TYR44 | CZ | 52.705 | 22.594 | 42.488 |
| 347 | TYR44 | OH | 51.679 | 23.093 | 41.717 |
| 348 | TYR44 | CE2 | 52.423 | 21.999 | 43.709 |
| 349 | TYR44 | CD2 | 53.461 | 21.53 | 44.504 |
| 350 | TYR44 | C | 56.566 | 23.504 | 45.543 |
| 351 | TYR44 | O | 57.341 | 23.715 | 44.603 |
| 352 | LEU45 | N | 55.823 | 24.453 | 46.09 |
| 353 | LEU45 | CA | 56.012 | 25.861 | 45.762 |
| 354 | LEU45 | CB | 54.913 | 26.321 | 44.796 |
| 355 | LEU45 | CG | 53.481 | 26.016 | 45.245 |
| 356 | LEU45 | CD1 | 52.931 | 27.067 | 46.208 |
| 357 | LEU45 | CD2 | 52.562 | 25.948 | 44.032 |
| 358 | LEU45 | C | 56.047 | 26.695 | 47.037 |
| 359 | LEU45 | O | 55.905 | 27.924 | 46.97 |
| 360 | ARG46 | N | 56.44 | 26.071 | 48.139 |
| 361 | ARG46 | CA | 56.299 | 26.696 | 49.46 |
| 362 | ARG46 | CB | 56.501 | 25.607 | 50.512 |
| 363 | ARG46 | CG | 56.421 | 26.122 | 51.948 |
| 364 | ARG46 | CD | 55.11 | 26.844 | 52.248 |
| 365 | ARG46 | NE | 53.936 | 25.991 | 52.018 |
| 366 | ARG46 | CZ | 52.882 | 26.412 | 51.316 |
| 367 | ARG46 | NH1 | 52.9 | 27.618 | 50.744 |
| 368 | ARG46 | NH2 | 51.828 | 25.615 | 51.156 |
| 369 | ARG46 | C | 57.258 | 27.862 | 49.697 |
| 370 | ARG46 | O | 56.849 | 28.838 | 50.336 |
| 371 | MET47 | N | 58.357 | 27.913 | 48.965 |
| 372 | MET47 | CA | 59.238 | 29.082 | 49.052 |
| 373 | MET47 | CB | 60.536 | 28.757 | 48.322 |
| 374 | MET47 | CG | 61.517 | 29.916 | 48.426 |
| 375 | MET47 | SD | 61.957 | 30.378 | 50.115 |
| 376 | MET47 | CE | 62.791 | 28.852 | 50.601 |
| 377 | MET47 | C | 58.601 | 30.334 | 48.436 |
| 378 | MET47 | O | 58.631 | 31.402 | 49.059 |
| 379 | SER48 | N | 57.803 | 30.148 | 47.396 |
| 380 | SER48 | CA | 57.133 | 31.289 | 46.774 |
| 381 | SER48 | CB | 56.86 | 30.972 | 45.311 |
| 382 | SER48 | OG | 58.116 | 30.786 | 44.673 |
| 383 | SER48 | C | 55.83 | 31.59 | 47.505 |
| 384 | SER48 | O | 55.477 | 32.762 | 47.664 |
| 385 | GLY49 | N | 55.289 | 30.572 | 48.156 |
| 386 | GLY49 | CA | 54.122 | 30.729 | 49.03 |
| 387 | GLY49 | C | 54.44 | 31.642 | 50.209 |
| 388 | GLY49 | O | 53.755 | 32.654 | 50.421 |
| 389 | ILE50 | N | 55.581 | 31.391 | 50.832 |
| 390 | ILE50 | CA | 56.039 | 32.211 | 51.955 |
| 391 | ILE50 | CB | 57.212 | 31.491 | 52.62 |
| 392 | ILE50 | CG2 | 57.839 | 32.354 | 53.706 |
| 393 | ILE50 | CG1 | 56.775 | 30.156 | 53.211 |
| 394 | ILE50 | CD1 | 55.746 | 30.343 | 54.322 |
| 395 | ILE50 | C | 56.467 | 33.607 | 51.499 |
| 396 | ILE50 | O | 56.145 | 34.583 | 52.187 |
| 397 | TYR51 | N | 56.915 | 33.728 | 50.258 |
| 398 | TYR51 | CA | 57.238 | 35.047 | 49.708 |
| 399 | TYR51 | CB | 57.986 | 34.871 | 48.389 |
| 400 | TYR51 | CG | 58.101 | 36.166 | 47.589 |
| 401 | TYR51 | CD1 | 58.85 | 37.227 | 48.082 |
| 402 | TYR51 | CE1 | 58.928 | 38.413 | 47.364 |
| 403 | TYR51 | CZ | 58.257 | 38.532 | 46.155 |
| 404 | TYR51 | OH | 58.276 | 39.731 | 45.477 |
| 405 | TYR51 | CE2 | 57.518 | 37.47 | 45.653 |
| 406 | TYR51 | CD2 | 57.44 | 36.285 | 46.373 |
| 407 | TYR51 | C | 55.988 | 35.895 | 49.472 |
| 408 | TYR51 | O | 55.978 | 37.062 | 49.884 |
| 409 | TRP52 | N | 54.895 | 35.273 | 49.054 |
| 410 | TRP52 | CA | 53.652 | 36.023 | 48.834 |
| 411 | TRP52 | CB | 52.609 | 35.138 | 48.154 |
| 412 | TRP52 | CG | 53.042 | 34.527 | 46.837 |
| 413 | TRP52 | CD1 | 53.845 | 35.097 | 45.873 |
| 414 | TRP52 | NE1 | 54.022 | 34.197 | 44.874 |
| 415 | TRP52 | CE2 | 53.354 | 33.054 | 45.124 |
| 416 | TRP52 | CZ2 | 53.269 | 31.845 | 44.45 |
| 417 | TRP52 | CH2 | 52.477 | 30.824 | 44.96 |
| 418 | TRP52 | CZ3 | 51.777 | 31.006 | 46.15 |
| 419 | TRP52 | CE3 | 51.874 | 32.206 | 46.843 |
| 420 | TRP52 | CD2 | 52.668 | 33.224 | 46.339 |
| 421 | TRP52 | C | 53.096 | 36.487 | 50.17 |
| 422 | TRP52 | O | 52.905 | 37.696 | 50.362 |
| 423 | GLY53 | N | 53.145 | 35.584 | 51.138 |
| 424 | GLY53 | CA | 52.709 | 35.871 | 52.509 |
| 425 | GLY53 | C | 53.46 | 37.048 | 53.128 |
| 426 | GLY53 | O | 52.844 | 38.072 | 53.451 |
| 427 | LEU54 | N | 54.78 | 36.988 | 53.09 |
| 428 | LEU54 | CA | 55.6 | 38.034 | 53.71 |
| 429 | LEU54 | CB | 57.049 | 37.572 | 53.706 |
| 430 | LEU54 | CG | 57.232 | 36.334 | 54.565 |
| 431 | LEU54 | CD1 | 58.663 | 35.833 | 54.479 |
| 432 | LEU54 | CD2 | 56.854 | 36.625 | 56.007 |
| 433 | LEU54 | C | 55.536 | 39.369 | 52.979 |
| 434 | LEU54 | O | 55.445 | 40.412 | 53.64 |
| 435 | THR55 | N | 55.363 | 39.342 | 51.67 |
| 436 | THR55 | CA | 55.323 | 40.601 | 50.935 |
| 437 | THR55 | CB | 55.593 | 40.341 | 49.459 |
| 438 | THR55 | OG1 | 56.87 | 39.73 | 49.354 |
| 439 | THR55 | CG2 | 55.634 | 41.644 | 48.67 |
| 440 | THR55 | C | 53.982 | 41.299 | 51.11 |
| 441 | THR55 | O | 53.987 | 42.498 | 51.413 |
| 442 | VAL56 | N | 52.906 | 40.539 | 51.253 |
| 443 | VAL56 | CA | 51.608 | 41.19 | 51.44 |
| 444 | VAL56 | CB | 50.467 | 40.277 | 50.972 |
| 445 | VAL56 | CG1 | 50.403 | 38.939 | 51.695 |
| 446 | VAL56 | CG2 | 49.121 | 40.977 | 51.078 |
| 447 | VAL56 | C | 51.427 | 41.652 | 52.887 |
| 448 | VAL56 | O | 50.965 | 42.784 | 53.085 |
| 449 | MET57 | N | 52.091 | 40.987 | 53.822 |
| 450 | MET57 | CA | 52.038 | 41.442 | 55.212 |
| 451 | MET57 | CB | 52.523 | 40.335 | 56.139 |
| 452 | MET57 | CG | 51.568 | 39.149 | 56.13 |
| 453 | MET57 | SD | 49.899 | 39.469 | 56.745 |
| 454 | MET57 | CE | 50.262 | 39.707 | 58.497 |
| 455 | MET57 | C | 52.899 | 42.679 | 55.401 |
| 456 | MET57 | O | 52.426 | 43.65 | 56.003 |
| 457 | ASP58 | N | 53.989 | 42.772 | 54.655 |
| 458 | ASP58 | CA | 54.839 | 43.956 | 54.766 |
| 459 | ASP58 | CB | 56.218 | 43.653 | 54.202 |
| 460 | ASP58 | CG | 57.167 | 44.759 | 54.65 |
| 461 | ASP58 | OD1 | 56.97 | 45.248 | 55.753 |
| 462 | ASP58 | OD2 | 58.092 | 45.063 | 53.912 |
| 463 | ASP58 | C | 54.246 | 45.156 | 54.031 |
| 464 | ASP58 | O | 54.287 | 46.257 | 54.589 |
| 465 | LEU59 | N | 53.452 | 44.91 | 53 |
| 466 | LEU59 | CA | 52.771 | 46.009 | 52.302 |
| 467 | LEU59 | CB | 52.25 | 45.506 | 50.959 |
| 468 | LEU59 | CG | 53.369 | 45.239 | 49.96 |
| 469 | LEU59 | CD1 | 52.825 | 44.57 | 48.703 |
| 470 | LEU59 | CD2 | 54.102 | 46.526 | 49.607 |
| 471 | LEU59 | C | 51.594 | 46.553 | 53.108 |
| 472 | LEU59 | O | 51.255 | 47.736 | 52.983 |
| 473 | MET60 | N | 51.069 | 45.742 | 54.012 |
| 474 | MET60 | CA | 50.021 | 46.2 | 54.925 |
| 475 | MET60 | CB | 49.055 | 45.044 | 55.153 |
| 476 | MET60 | CG | 48.399 | 44.625 | 53.843 |
| 477 | MET60 | SD | 47.168 | 43.309 | 53.965 |
| 478 | MET60 | CE | 48.227 | 41.986 | 54.584 |
| 479 | MET60 | C | 50.572 | 46.705 | 56.264 |
| 480 | MET60 | O | 49.784 | 46.979 | 57.179 |
| 481 | GLY61 | N | 51.891 | 46.703 | 56.42 |
| 482 | GLY61 | CA | 52.545 | 47.214 | 57.638 |
| 483 | GLY61 | C | 52.737 | 46.155 | 58.726 |
| 484 | GLY61 | O | 53.5 | 46.35 | 59.68 |
| 485 | GLN62 | N | 52.182 | 44.983 | 58.481 |
| 486 | GLN62 | CA | 52.059 | 43.944 | 59.499 |
| 487 | GLN62 | CB | 50.598 | 43.525 | 59.517 |
| 488 | GLN62 | CG | 49.755 | 44.728 | 59.924 |
| 489 | GLN62 | CD | 48.289 | 44.513 | 59.582 |
| 490 | GLN62 | OE1 | 47.583 | 43.74 | 60.239 |
| 491 | GLN62 | NE2 | 47.83 | 45.26 | 58.593 |
| 492 | GLN62 | C | 52.983 | 42.762 | 59.24 |
| 493 | GLN62 | O | 52.645 | 41.609 | 59.536 |
| 494 | LEU63 | N | 54.229 | 43.079 | 58.919 |
| 495 | LEU63 | CA | 55.242 | 42.036 | 58.699 |
| 496 | LEU63 | CB | 56.409 | 42.643 | 57.928 |
| 497 | LEU63 | CG | 57.451 | 41.593 | 57.556 |
| 498 | LEU63 | CD1 | 56.839 | 40.503 | 56.683 |
| 499 | LEU63 | CD2 | 58.651 | 42.226 | 56.86 |
| 500 | LEU63 | C | 55.748 | 41.478 | 60.033 |
| 501 | LEU63 | O | 56.136 | 40.305 | 60.106 |
| 502 | HIS64 | N | 55.418 | 42.196 | 61.097 |
| 503 | HIS64 | CA | 55.759 | 41.82 | 62.472 |
| 504 | HIS64 | CB | 55.662 | 43.082 | 63.332 |
| 505 | HIS64 | CG | 54.331 | 43.818 | 63.248 |
| 506 | HIS64 | ND1 | 54.087 | 44.973 | 62.597 |
| 507 | HIS64 | CE1 | 52.789 | 45.306 | 62.751 |
| 508 | HIS64 | NE2 | 52.211 | 44.358 | 63.523 |
| 509 | HIS64 | CD2 | 53.151 | 43.443 | 63.847 |
| 510 | HIS64 | C | 54.848 | 40.729 | 63.052 |
| 511 | HIS64 | O | 55.036 | 40.327 | 64.205 |
| 512 | ARG65 | N | 53.862 | 40.283 | 62.286 |
| 513 | ARG65 | CA | 53.015 | 39.171 | 62.716 |
| 514 | ARG65 | CB | 51.613 | 39.387 | 62.159 |
| 515 | ARG65 | CG | 50.974 | 40.658 | 62.703 |
| 516 | ARG65 | CD | 49.588 | 40.867 | 62.105 |
| 517 | ARG65 | NE | 48.736 | 39.69 | 62.339 |
| 518 | ARG65 | CZ | 47.448 | 39.639 | 61.992 |
| 519 | ARG65 | NH1 | 46.868 | 40.699 | 61.427 |
| 520 | ARG65 | NH2 | 46.734 | 38.536 | 62.23 |
| 521 | ARG65 | C | 53.545 | 37.831 | 62.207 |
| 522 | ARG65 | O | 53.041 | 36.775 | 62.606 |
| 523 | MET66 | N | 54.553 | 37.873 | 61.351 |
| 524 | MET66 | CA | 55.078 | 36.644 | 60.75 |
| 525 | MET66 | CB | 55.528 | 36.967 | 59.334 |
| 526 | MET66 | CG | 54.366 | 37.52 | 58.515 |
| 527 | MET66 | SD | 52.934 | 36.427 | 58.341 |
| 528 | MET66 | CE | 53.706 | 35.061 | 57.443 |
| 529 | MET66 | C | 56.223 | 36.029 | 61.553 |
| 530 | MET66 | O | 56.938 | 36.709 | 62.301 |
| 531 | ASN67 | N | 56.396 | 34.731 | 61.363 |
| 532 | ASN67 | CA | 57.419 | 33.95 | 62.078 |
| 533 | ASN67 | CB | 56.968 | 32.491 | 62.088 |
| 534 | ASN67 | CG | 55.602 | 32.344 | 62.76 |
| 535 | ASN67 | OD1 | 54.558 | 32.415 | 62.102 |
| 536 | ASN67 | ND2 | 55.637 | 32.042 | 64.045 |
| 537 | ASN67 | C | 58.79 | 34.037 | 61.406 |
| 538 | ASN67 | O | 59.329 | 33.013 | 60.961 |
| 539 | ARG68 | N | 59.44 | 35.178 | 61.58 |
| 540 | ARG68 | CA | 60.693 | 35.5 | 60.876 |
| 541 | ARG68 | CB | 61.153 | 36.87 | 61.353 |
| 542 | ARG68 | CG | 62.48 | 37.255 | 60.71 |
| 543 | ARG68 | CD | 63.122 | 38.42 | 61.448 |
| 544 | ARG68 | NE | 63.297 | 38.068 | 62.867 |
| 545 | ARG68 | CZ | 64.426 | 37.568 | 63.379 |
| 546 | ARG68 | NH1 | 64.456 | 37.166 | 64.651 |
| 547 | ARG68 | NH2 | 65.493 | 37.383 | 62.598 |
| 548 | ARG68 | C | 61.835 | 34.516 | 61.117 |
| 549 | ARG68 | O | 62.373 | 33.979 | 60.143 |
| 550 | GLU69 | N | 62.01 | 34.073 | 62.353 |
| 551 | GLU69 | CA | 63.126 | 33.17 | 62.662 |
| 552 | GLU69 | CB | 63.289 | 33.126 | 64.177 |
| 553 | GLU69 | CG | 64.43 | 32.206 | 64.599 |
| 554 | GLU69 | CD | 64.48 | 32.111 | 66.121 |
| 555 | GLU69 | OE1 | 65.118 | 32.963 | 66.721 |
| 556 | GLU69 | OE2 | 63.754 | 31.283 | 66.654 |
| 557 | GLU69 | C | 62.904 | 31.748 | 62.138 |
| 558 | GLU69 | O | 63.856 | 31.121 | 61.657 |
| 559 | GLU70 | N | 61.649 | 31.372 | 61.96 |
| 560 | GLU70 | CA | 61.345 | 30.023 | 61.485 |
| 561 | GLU70 | CB | 59.984 | 29.637 | 62.044 |
| 562 | GLU70 | CG | 60.063 | 29.553 | 63.564 |
| 563 | GLU70 | CD | 58.671 | 29.616 | 64.179 |
| 564 | GLU70 | OE1 | 58.02 | 28.586 | 64.258 |
| 565 | GLU70 | OE2 | 58.276 | 30.718 | 64.545 |
| 566 | GLU70 | C | 61.347 | 29.991 | 59.963 |
| 567 | GLU70 | O | 61.813 | 29.012 | 59.366 |
| 568 | ILE71 | N | 61.122 | 31.154 | 59.376 |
| 569 | ILE71 | CA | 61.21 | 31.298 | 57.926 |
| 570 | ILE71 | CB | 60.431 | 32.55 | 57.542 |
| 571 | ILE71 | CG2 | 60.629 | 32.888 | 56.069 |
| 572 | ILE71 | CG1 | 58.954 | 32.362 | 57.862 |
| 573 | ILE71 | CD1 | 58.158 | 33.637 | 57.617 |
| 574 | ILE71 | C | 62.665 | 31.422 | 57.49 |
| 575 | ILE71 | O | 63.06 | 30.787 | 56.506 |
| 576 | LEU72 | N | 63.491 | 31.955 | 58.375 |
| 577 | LEU72 | CA | 64.928 | 32.059 | 58.103 |
| 578 | LEU72 | CB | 65.554 | 32.999 | 59.131 |
| 579 | LEU72 | CG | 65.957 | 34.352 | 58.546 |
| 580 | LEU72 | CD1 | 64.79 | 35.083 | 57.889 |
| 581 | LEU72 | CD2 | 66.588 | 35.228 | 59.622 |
| 582 | LEU72 | C | 65.598 | 30.693 | 58.195 |
| 583 | LEU72 | O | 66.353 | 30.324 | 57.285 |
| 584 | ALA73 | N | 65.113 | 29.864 | 59.108 |
| 585 | ALA73 | CA | 65.634 | 28.5 | 59.226 |
| 586 | ALA73 | CB | 65.173 | 27.918 | 60.557 |
| 587 | ALA73 | C | 65.149 | 27.611 | 58.083 |
| 588 | ALA73 | O | 65.958 | 26.872 | 57.505 |
| 589 | PHE74 | N | 63.949 | 27.887 | 57.594 |
| 590 | PHE74 | CA | 63.408 | 27.149 | 56.451 |
| 591 | PHE74 | CB | 61.937 | 27.527 | 56.291 |
| 592 | PHE74 | CG | 61.237 | 26.906 | 55.084 |
| 593 | PHE74 | CD1 | 61.015 | 25.536 | 55.034 |
| 594 | PHE74 | CE1 | 60.377 | 24.975 | 53.935 |
| 595 | PHE74 | CZ | 59.959 | 25.784 | 52.886 |
| 596 | PHE74 | CE2 | 60.178 | 27.155 | 52.937 |
| 597 | PHE74 | CD2 | 60.817 | 27.715 | 54.035 |
| 598 | PHE74 | C | 64.165 | 27.476 | 55.168 |
| 599 | PHE74 | O | 64.663 | 26.549 | 54.521 |
| 600 | ILE75 | N | 64.508 | 28.741 | 54.982 |
| 601 | ILE75 | CA | 65.232 | 29.158 | 53.775 |
| 602 | ILE75 | CB | 65.159 | 30.676 | 53.689 |
| 603 | ILE75 | CG2 | 66.016 | 31.212 | 52.551 |
| 604 | ILE75 | CG1 | 63.722 | 31.135 | 53.515 |
| 605 | ILE75 | CD1 | 63.658 | 32.651 | 53.45 |
| 606 | ILE75 | C | 66.694 | 28.721 | 53.789 |
| 607 | ILE75 | O | 67.193 | 28.237 | 52.763 |
| 608 | LYS76 | N | 67.263 | 28.61 | 54.979 |
| 609 | LYS76 | CA | 68.647 | 28.15 | 55.095 |
| 610 | LYS76 | CB | 69.14 | 28.518 | 56.489 |
| 611 | LYS76 | CG | 70.616 | 28.191 | 56.67 |
| 612 | LYS76 | CD | 71.106 | 28.629 | 58.044 |
| 613 | LYS76 | CE | 72.593 | 28.343 | 58.213 |
| 614 | LYS76 | NZ | 73.067 | 28.789 | 59.533 |
| 615 | LYS76 | C | 68.747 | 26.639 | 54.881 |
| 616 | LYS76 | O | 69.69 | 26.176 | 54.23 |
| 617 | SER77 | N | 67.661 | 25.939 | 55.168 |
| 618 | SER77 | CA | 67.589 | 24.495 | 54.929 |
| 619 | SER77 | CB | 66.669 | 23.893 | 55.981 |
| 620 | SER77 | OG | 67.19 | 24.24 | 57.256 |
| 621 | SER77 | C | 67.064 | 24.157 | 53.53 |
| 622 | SER77 | O | 66.946 | 22.976 | 53.178 |
| 623 | CYS78 | N | 66.704 | 25.174 | 52.763 |
| 624 | CYS78 | CA | 66.282 | 24.96 | 51.383 |
| 625 | CYS78 | CB | 65.125 | 25.89 | 51.046 |
| 626 | CYS78 | SG | 63.546 | 25.496 | 51.825 |
| 627 | CYS78 | C | 67.413 | 25.203 | 50.395 |
| 628 | CYS78 | O | 67.296 | 24.783 | 49.238 |
| 629 | GLN79 | N | 68.482 | 25.863 | 50.812 |
| 630 | GLN79 | CA | 69.61 | 26.021 | 49.888 |
| 631 | GLN79 | CB | 70.543 | 27.143 | 50.334 |
| 632 | GLN79 | CG | 71.732 | 27.223 | 49.377 |
| 633 | GLN79 | CD | 72.624 | 28.427 | 49.635 |
| 634 | GLN79 | OE1 | 73.014 | 28.723 | 50.774 |
| 635 | GLN79 | NE2 | 72.908 | 29.13 | 48.555 |
| 636 | GLN79 | C | 70.395 | 24.72 | 49.779 |
| 637 | GLN79 | O | 70.88 | 24.178 | 50.777 |
| 638 | HIS80 | N | 70.483 | 24.21 | 48.565 |
| 639 | HIS80 | CA | 71.26 | 22.997 | 48.324 |
| 640 | HIS80 | CB | 70.639 | 22.196 | 47.183 |
| 641 | HIS80 | CG | 69.405 | 21.404 | 47.59 |
| 642 | HIS80 | ND1 | 69.162 | 20.112 | 47.303 |
| 643 | HIS80 | CE1 | 67.982 | 19.747 | 47.841 |
| 644 | HIS80 | NE2 | 67.467 | 20.825 | 48.474 |
| 645 | HIS80 | CD2 | 68.332 | 21.854 | 48.325 |
| 646 | HIS80 | C | 72.713 | 23.351 | 48.04 |
| 647 | HIS80 | O | 73.042 | 24.509 | 47.757 |
| 648 | GLU81 | N | 73.548 | 22.326 | 47.956 |
| 649 | GLU81 | CA | 75.004 | 22.519 | 47.798 |
| 650 | GLU81 | CB | 75.712 | 21.209 | 48.129 |
| 651 | GLU81 | CG | 75.549 | 20.82 | 49.595 |
| 652 | GLU81 | CD | 76.197 | 21.864 | 50.505 |
| 653 | GLU81 | OE1 | 75.453 | 22.659 | 51.062 |
| 654 | GLU81 | OE2 | 77.393 | 21.756 | 50.732 |
| 655 | GLU81 | C | 75.453 | 22.983 | 46.406 |
| 656 | GLU81 | O | 76.638 | 23.27 | 46.213 |
| 657 | CYS82 | N | 74.526 | 23.093 | 45.468 |
| 658 | CYS82 | CA | 74.834 | 23.662 | 44.155 |
| 659 | CYS82 | CB | 74.071 | 22.89 | 43.087 |
| 660 | CYS82 | SG | 72.273 | 22.938 | 43.24 |
| 661 | CYS82 | C | 74.455 | 25.144 | 44.092 |
| 662 | CYS82 | O | 74.459 | 25.739 | 43.008 |
| 663 | GLY83 | N | 73.977 | 25.683 | 45.203 |
| 664 | GLY83 | CA | 73.634 | 27.104 | 45.265 |
| 665 | GLY83 | C | 72.135 | 27.333 | 45.248 |
| 666 | GLY83 | O | 71.602 | 28.124 | 46.041 |
| 667 | GLY84 | N | 71.498 | 26.683 | 44.289 |
| 668 | GLY84 | CA | 70.053 | 26.769 | 44.085 |
| 669 | GLY84 | C | 69.241 | 26.441 | 45.322 |
| 670 | GLY84 | O | 69.542 | 25.515 | 46.088 |
| 671 | ILE85 | N | 68.202 | 27.232 | 45.497 |
| 672 | ILE85 | CA | 67.308 | 27.072 | 46.629 |
| 673 | ILE85 | CB | 66.971 | 28.473 | 47.123 |
| 674 | ILE85 | CG2 | 66.144 | 28.43 | 48.403 |
| 675 | ILE85 | CG1 | 68.274 | 29.233 | 47.357 |
| 676 | ILE85 | CD1 | 68.041 | 30.711 | 47.635 |
| 677 | ILE85 | C | 66.077 | 26.306 | 46.165 |
| 678 | ILE85 | O | 65.594 | 26.501 | 45.04 |
| 679 | SER86 | N | 65.767 | 25.27 | 46.919 |
| 680 | SER86 | CA | 64.601 | 24.433 | 46.656 |
| 681 | SER86 | CB | 64.751 | 23.131 | 47.425 |
| 682 | SER86 | OG | 64.727 | 23.429 | 48.813 |
| 683 | SER86 | C | 63.322 | 25.123 | 47.103 |
| 684 | SER86 | O | 63.343 | 26.102 | 47.857 |
| 685 | ALA87 | N | 62.208 | 24.567 | 46.659 |
| 686 | ALA87 | CA | 60.884 | 25.1 | 47.009 |
| 687 | ALA87 | CB | 59.902 | 24.587 | 45.976 |
| 688 | ALA87 | C | 60.403 | 24.641 | 48.38 |
| 689 | ALA87 | O | 59.413 | 25.159 | 48.912 |
| 690 | SER88 | N | 61.093 | 23.642 | 48.898 |
| 691 | SER88 | CA | 60.869 | 23.084 | 50.228 |
| 692 | SER88 | CB | 59.593 | 22.255 | 50.214 |
| 693 | SER88 | OG | 59.457 | 21.603 | 51.467 |
| 694 | SER88 | C | 62.07 | 22.204 | 50.522 |
| 695 | SER88 | O | 62.657 | 21.673 | 49.574 |
| 696 | ILE89 | N | 62.447 | 22.078 | 51.784 |
| 697 | ILE89 | CA | 63.637 | 21.303 | 52.175 |
| 698 | ILE89 | CB | 63.595 | 21.164 | 53.694 |
| 699 | ILE89 | CG2 | 64.839 | 20.455 | 54.22 |
| 700 | ILE89 | CG1 | 63.454 | 22.535 | 54.346 |
| 701 | ILE89 | CD1 | 63.31 | 22.421 | 55.86 |
| 702 | ILE89 | C | 63.661 | 19.916 | 51.523 |
| 703 | ILE89 | O | 62.649 | 19.204 | 51.525 |
| 704 | GLY90 | N | 64.719 | 19.663 | 50.765 |
| 705 | GLY90 | CA | 64.898 | 18.359 | 50.11 |
| 706 | GLY90 | C | 64.479 | 18.336 | 48.635 |
| 707 | GLY90 | O | 64.821 | 17.395 | 47.909 |
| 708 | HIS91 | N | 63.726 | 19.338 | 48.213 |
| 709 | HIS91 | CA | 63.198 | 19.393 | 46.841 |
| 710 | HIS91 | CB | 61.998 | 20.342 | 46.779 |
| 711 | HIS91 | CG | 60.687 | 19.876 | 47.404 |
| 712 | HIS91 | ND1 | 60.497 | 19.209 | 48.563 |
| 713 | HIS91 | CE1 | 59.179 | 18.995 | 48.748 |
| 714 | HIS91 | NE2 | 58.526 | 19.544 | 47.701 |
| 715 | HIS91 | CD2 | 59.439 | 20.093 | 46.869 |
| 716 | HIS91 | C | 64.267 | 19.88 | 45.871 |
| 717 | HIS91 | O | 65.34 | 20.322 | 46.291 |
| 718 | ASP92 | N | 63.974 | 19.799 | 44.585 |
| 719 | ASP92 | CA | 64.925 | 20.278 | 43.567 |
| 720 | ASP92 | CB | 64.394 | 19.995 | 42.159 |
| 721 | ASP92 | CG | 64.699 | 18.567 | 41.702 |
| 722 | ASP92 | OD1 | 64.601 | 17.666 | 42.524 |
| 723 | ASP92 | OD2 | 64.959 | 18.397 | 40.517 |
| 724 | ASP92 | C | 65.189 | 21.775 | 43.704 |
| 725 | ASP92 | O | 64.275 | 22.564 | 43.98 |
| 726 | PRO93 | N | 66.465 | 22.115 | 43.641 |
| 727 | PRO93 | CA | 66.889 | 23.507 | 43.494 |
| 728 | PRO93 | CB | 68.384 | 23.466 | 43.524 |
| 729 | PRO93 | CG | 68.846 | 22.019 | 43.568 |
| 730 | PRO93 | CD | 67.586 | 21.175 | 43.579 |
| 731 | PRO93 | C | 66.371 | 24.097 | 42.186 |
| 732 | PRO93 | O | 66.435 | 23.466 | 41.122 |
| 733 | HIS94 | N | 65.828 | 25.295 | 42.293 |
| 734 | HIS94 | CA | 65.232 | 25.945 | 41.128 |
| 735 | HIS94 | CB | 63.742 | 25.638 | 41.179 |
| 736 | HIS94 | CG | 63.023 | 25.69 | 39.85 |
| 737 | HIS94 | ND1 | 62.769 | 24.639 | 39.052 |
| 738 | HIS94 | CE1 | 62.107 | 25.06 | 37.957 |
| 739 | HIS94 | NE2 | 61.937 | 26.396 | 38.069 |
| 740 | HIS94 | CD2 | 62.491 | 26.797 | 39.235 |
| 741 | HIS94 | C | 65.467 | 27.449 | 41.193 |
| 742 | HIS94 | O | 65.287 | 28.058 | 42.252 |
| 743 | LEU95 | N | 65.691 | 28.067 | 40.045 |
| 744 | LEU95 | CA | 65.985 | 29.507 | 39.993 |
| 745 | LEU95 | CB | 66.46 | 29.808 | 38.576 |
| 746 | LEU95 | CG | 67.029 | 31.211 | 38.422 |
| 747 | LEU95 | CD1 | 68.116 | 31.481 | 39.457 |
| 748 | LEU95 | CD2 | 67.575 | 31.402 | 37.013 |
| 749 | LEU95 | C | 64.789 | 30.401 | 40.352 |
| 750 | LEU95 | O | 64.993 | 31.47 | 40.936 |
| 751 | LEU96 | N | 63.582 | 29.863 | 40.274 |
| 752 | LEU96 | CA | 62.395 | 30.616 | 40.696 |
| 753 | LEU96 | CB | 61.168 | 29.902 | 40.139 |
| 754 | LEU96 | CG | 59.862 | 30.546 | 40.589 |
| 755 | LEU96 | CD1 | 59.724 | 31.958 | 40.03 |
| 756 | LEU96 | CD2 | 58.672 | 29.689 | 40.174 |
| 757 | LEU96 | C | 62.284 | 30.678 | 42.22 |
| 758 | LEU96 | O | 62.025 | 31.751 | 42.78 |
| 759 | TYR97 | N | 62.747 | 29.629 | 42.88 |
| 760 | TYR97 | CA | 62.669 | 29.577 | 44.339 |
| 761 | TYR97 | CB | 62.431 | 28.135 | 44.759 |
| 762 | TYR97 | CG | 61.13 | 27.578 | 44.188 |
| 763 | TYR97 | CD1 | 61.161 | 26.567 | 43.235 |
| 764 | TYR97 | CE1 | 59.976 | 26.067 | 42.712 |
| 765 | TYR97 | CZ | 58.762 | 26.579 | 43.146 |
| 766 | TYR97 | OH | 57.586 | 26.021 | 42.696 |
| 767 | TYR97 | CE2 | 58.726 | 27.593 | 44.094 |
| 768 | TYR97 | CD2 | 59.913 | 28.093 | 44.615 |
| 769 | TYR97 | C | 63.943 | 30.141 | 44.95 |
| 770 | TYR97 | O | 63.916 | 30.697 | 46.055 |
| 771 | THR98 | N | 64.964 | 30.233 | 44.116 |
| 772 | THR98 | CA | 66.181 | 30.952 | 44.481 |
| 773 | THR98 | CB | 67.272 | 30.614 | 43.468 |
| 774 | THR98 | OG1 | 67.564 | 29.227 | 43.573 |
| 775 | THR98 | CG2 | 68.558 | 31.379 | 43.746 |
| 776 | THR98 | C | 65.901 | 32.45 | 44.478 |
| 111 | THR98 | O | 66.176 | 33.118 | 45.483 |
| 778 | LEU99 | N | 65.101 | 32.889 | 43.517 |
| 779 | LEU99 | CA | 64.678 | 34.289 | 43.466 |
| 780 | LEU99 | CB | 63.958 | 34.543 | 42.146 |
| 781 | LEU99 | CG | 63.39 | 35.957 | 42.095 |
| 782 | LEU99 | CD1 | 64.49 | 37.003 | 42.215 |
| 783 | LEU99 | CD2 | 62.563 | 36.189 | 40.836 |
| 784 | LEU99 | C | 63.738 | 34.622 | 44.618 |
| 785 | LEU99 | O | 64.053 | 35.543 | 45.381 |
| 786 | SER100 | N | 62.825 | 33.714 | 44.925 |
| 787 | SER100 | CA | 61.867 | 33.947 | 46.013 |
| 788 | SER100 | CB | 60.834 | 32.826 | 46.006 |
| 789 | SER100 | OG | 60.151 | 32.859 | 44.76 |
| 790 | SER100 | C | 62.542 | 34.001 | 47.382 |
| 791 | SER100 | O | 62.311 | 34.963 | 48.125 |
| 792 | ALA101 | N | 63.558 | 33.177 | 47.588 |
| 793 | ALA101 | CA | 64.267 | 33.192 | 48.869 |
| 794 | ALA101 | CB | 65.054 | 31.9 | 49.01 |
| 795 | ALA101 | C | 65.217 | 34.377 | 48.999 |
| 796 | ALA101 | O | 65.276 | 34.976 | 50.079 |
| 797 | VAL102 | N | 65.722 | 34.871 | 47.88 |
| 798 | VAL102 | CA | 66.559 | 36.074 | 47.913 |
| 799 | VAL102 | CB | 67.356 | 36.16 | 46.614 |
| 800 | VAL102 | CG1 | 68.001 | 37.529 | 46.427 |
| 801 | VAL102 | CG2 | 68.409 | 35.059 | 46.548 |
| 802 | VAL102 | C | 65.708 | 37.328 | 48.103 |
| 803 | VAL102 | O | 66.104 | 38.212 | 48.872 |
| 804 | GLN103 | N | 64.458 | 37.273 | 47.675 |
| 805 | GLN103 | CA | 63.549 | 38.394 | 47.906 |
| 806 | GLN103 | CB | 62.376 | 38.267 | 46.948 |
| 807 | GLN103 | CG | 62.841 | 38.34 | 45.502 |
| 808 | GLN103 | CD | 61.654 | 38.188 | 44.562 |
| 809 | GLN103 | OE1 | 61.201 | 37.072 | 44.272 |
| 810 | GLN103 | NE2 | 61.181 | 39.323 | 44.08 |
| 811 | GLN103 | C | 63.037 | 38.409 | 49.342 |
| 812 | GLN103 | O | 62.981 | 39.486 | 49.948 |
| 813 | ILE104 | N | 62.94 | 37.239 | 49.954 |
| 814 | ILE104 | CA | 62.553 | 37.173 | 51.366 |
| 815 | ILE104 | CB | 62.145 | 35.746 | 51.702 |
| 816 | ILE104 | CG2 | 61.878 | 35.616 | 53.195 |
| 817 | ILE104 | CG1 | 60.923 | 35.313 | 50.907 |
| 818 | ILE104 | CD1 | 60.579 | 33.855 | 51.189 |
| 819 | ILE104 | C | 63.707 | 37.577 | 52.279 |
| 820 | ILE104 | O | 63.497 | 38.348 | 53.224 |
| 821 | LEU105 | N | 64.926 | 37.287 | 51.855 |
| 822 | LEU105 | CA | 66.092 | 37.695 | 52.639 |
| 823 | LEU105 | CB | 67.258 | 36.773 | 52.317 |
| 824 | LEU105 | CG | 66.981 | 35.337 | 52.746 |
| 825 | LEU105 | CD1 | 68.155 | 34.439 | 52.383 |
| 826 | LEU105 | CD2 | 66.68 | 35.242 | 54.239 |
| 827 | LEU105 | C | 66.48 | 39.149 | 52.38 |
| 828 | LEU105 | O | 67.214 | 39.741 | 53.178 |
| 829 | THR106 | N | 65.918 | 39.757 | 51.351 |
| 830 | THR106 | CA | 66.075 | 41.198 | 51.177 |
| 831 | THR106 | CB | 65.913 | 41.527 | 49.696 |
| 832 | THR106 | OG1 | 66.984 | 40.913 | 48.992 |
| 833 | THR106 | CG2 | 65.982 | 43.026 | 49.433 |
| 834 | THR106 | C | 65.017 | 41.928 | 51.999 |
| 835 | THR106 | O | 65.346 | 42.876 | 52.723 |
| 836 | LEU107 | N | 63.865 | 41.287 | 52.128 |
| 837 | LEU107 | CA | 62.733 | 41.856 | 52.867 |
| 838 | LEU107 | CB | 61.511 | 41.017 | 52.506 |
| 839 | LEU107 | CG | 60.217 | 41.625 | 53.024 |
| 840 | LEU107 | CD1 | 60 | 42.995 | 52.401 |
| 841 | LEU107 | CD2 | 59.037 | 40.711 | 52.719 |
| 842 | LEU107 | C | 62.949 | 41.81 | 54.381 |
| 843 | LEU107 | O | 62.571 | 42.746 | 55.094 |
| 844 | TYR108 | N | 63.632 | 40.778 | 54.846 |
| 845 | TYR108 | CA | 64.003 | 40.685 | 56.263 |
| 846 | TYR108 | CB | 63.937 | 39.224 | 56.692 |
| 847 | TYR108 | CG | 62.548 | 38.729 | 57.086 |
| 848 | TYR108 | CD1 | 62.162 | 37.427 | 56.793 |
| 849 | TYR108 | CE1 | 60.907 | 36.971 | 57.177 |
| 850 | TYR108 | CZ | 60.041 | 37.822 | 57.852 |
| 851 | TYR108 | OH | 58.87 | 37.325 | 58.382 |
| 852 | TYR108 | CE2 | 60.417 | 39.128 | 58.129 |
| 853 | TYR108 | CD2 | 61.672 | 39.582 | 57.746 |
| 854 | TYR108 | C | 65.4 | 41.226 | 56.565 |
| 855 | TYR108 | O | 65.791 | 41.261 | 57.738 |
| 856 | ASP109 | N | 66.091 | 41.717 | 55.543 |
| 857 | ASP109 | CA | 67.51 | 42.101 | 55.642 |
| 858 | ASP109 | CB | 67.635 | 43.436 | 56.369 |
| 859 | ASP109 | CG | 69.061 | 43.959 | 56.234 |
| 860 | ASP109 | OD1 | 69.698 | 43.612 | 55.249 |
| 861 | ASP109 | OD2 | 69.506 | 44.65 | 57.139 |
| 862 | ASP109 | C | 68.305 | 41.002 | 56.352 |
| 863 | ASP109 | O | 68.882 | 41.186 | 57.431 |
| 864 | SER110 | N | 68.314 | 39.848 | 55.712 |
| 865 | SER110 | CA | 68.906 | 38.639 | 56.276 |
| 866 | SER110 | CB | 67.822 | 37.86 | 57.011 |
| 867 | SER110 | OG | 67.286 | 38.693 | 58.032 |
| 868 | SER110 | C | 69.486 | 37.772 | 55.168 |
| 869 | SER110 | O | 69.478 | 36.538 | 55.266 |
| 870 | ILE111 | N | 70.163 | 38.414 | 54.227 |
| 871 | ILE111 | CA | 70.766 | 37.686 | 53.099 |
| 872 | ILE111 | CB | 71.137 | 38.684 | 52.001 |
| 873 | ILE111 | CG2 | 69.905 | 39.172 | 51.249 |
| 874 | ILE111 | CG1 | 71.921 | 39.865 | 52.566 |
| 875 | ILE111 | CD1 | 72.294 | 40.859 | 51.474 |
| 876 | ILE111 | C | 72.004 | 36.881 | 53.507 |
| 877 | ILE111 | O | 72.225 | 35.809 | 52.933 |
| 878 | ASN112 | N | 72.515 | 37.178 | 54.695 |
| 879 | ASN112 | CA | 73.7 | 36.531 | 55.268 |
| 880 | ASN112 | CB | 74.252 | 37.463 | 56.345 |
| 881 | ASN112 | CG | 74.176 | 38.93 | 55.915 |
| 882 | ASN112 | OD1 | 74.607 | 39.307 | 54.818 |
| 883 | ASN112 | ND2 | 73.6 | 39.743 | 56.787 |
| 884 | ASN112 | C | 73.374 | 35.185 | 55.927 |
| 885 | ASN112 | O | 74.259 | 34.549 | 56.511 |
| 886 | VAL113 | N | 72.109 | 34.789 | 55.888 |
| 887 | VAL113 | CA | 71.699 | 33.489 | 56.425 |
| 888 | VAL113 | CB | 70.24 | 33.614 | 56.865 |
| 889 | VAL113 | CG1 | 69.665 | 32.297 | 57.378 |
| 890 | VAL113 | CG2 | 70.095 | 34.697 | 57.927 |
| 891 | VAL113 | C | 71.859 | 32.405 | 55.357 |
| 892 | VAL113 | O | 71.957 | 31.212 | 55.671 |
| 893 | ILE114 | N | 72.005 | 32.836 | 54.115 |
| 894 | ILE114 | CA | 72.216 | 31.892 | 53.021 |
| 895 | ILE114 | CB | 70.98 | 31.96 | 52.127 |
| 896 | ILE114 | CG2 | 71.214 | 32.787 | 50.863 |
| 897 | ILE114 | CG1 | 70.51 | 30.556 | 51.777 |
| 898 | ILE114 | CD1 | 69.216 | 30.587 | 50.981 |
| 899 | ILE114 | C | 73.518 | 32.233 | 52.289 |
| 900 | ILE114 | O | 74.014 | 33.362 | 52.387 |
| 901 | ASP115 | N | 74.135 | 31.239 | 51.672 |
| 902 | ASP115 | CA | 75.386 | 31.489 | 50.952 |
| 903 | ASP115 | CB | 76.126 | 30.165 | 50.768 |
| 904 | ASP115 | CG | 77.567 | 30.411 | 50.329 |
| 905 | ASP115 | OD1 | 78.464 | 30.038 | 51.068 |
| 906 | ASP115 | OD2 | 77.743 | 31.011 | 49.274 |
| 907 | ASP115 | C | 75.088 | 32.152 | 49.606 |
| 908 | ASP115 | O | 74.808 | 31.484 | 48.599 |
| 909 | VAL116 | N | 75.373 | 33.444 | 49.562 |
| 910 | VAL116 | CA | 75.068 | 34.274 | 48.392 |
| 911 | VAL116 | CB | 75.13 | 35.733 | 48.848 |
| 912 | VAL116 | CG1 | 76.289 | 35.988 | 49.807 |
| 913 | VAL116 | CG2 | 75.168 | 36.708 | 47.676 |
| 914 | VAL116 | C | 76.003 | 34.044 | 47.203 |
| 915 | VAL116 | O | 75.519 | 34.038 | 46.064 |
| 916 | ASN117 | N | 77.187 | 33.513 | 47.457 |
| 917 | ASN117 | CA | 78.139 | 33.274 | 46.369 |
| 918 | ASN117 | CB | 79.538 | 33.235 | 46.968 |
| 919 | ASN117 | CG | 79.834 | 34.579 | 47.627 |
| 920 | ASN117 | OD1 | 79.688 | 34.745 | 48.845 |
| 921 | ASN117 | ND2 | 80.167 | 35.549 | 46.793 |
| 922 | ASN117 | C | 77.83 | 31.965 | 45.658 |
| 923 | ASN117 | O | 77.951 | 31.886 | 44.429 |
| 924 | LYS118 | N | 77.129 | 31.1 | 46.371 |
| 925 | LYS118 | CA | 76.681 | 29.834 | 45.806 |
| 926 | LYS118 | CB | 76.4 | 28.895 | 46.974 |
| 927 | LYS118 | CG | 76.771 | 27.451 | 46.66 |
| 928 | LYS118 | CD | 78.277 | 27.271 | 46.519 |
| 929 | LYS118 | CE | 78.997 | 27.572 | 47.83 |
| 930 | LYS118 | NZ | 78.563 | 26.65 | 48.892 |
| 931 | LYS118 | C | 75.412 | 30.063 | 44.988 |
| 932 | LYS118 | O | 75.271 | 29.492 | 43.899 |
| 933 | VAL119 | N | 74.662 | 31.09 | 45.363 |
| 934 | VAL119 | CA | 73.474 | 31.483 | 44.596 |
| 935 | VAL119 | CB | 72.665 | 32.485 | 45.415 |
| 936 | VAL119 | CG1 | 71.565 | 33.126 | 44.582 |
| 937 | VAL119 | CG2 | 72.081 | 31.859 | 46.671 |
| 938 | VAL119 | C | 73.883 | 32.145 | 43.284 |
| 939 | VAL119 | O | 73.381 | 31.756 | 42.22 |
| 940 | VAL120 | N | 74.981 | 32.885 | 43.333 |
| 941 | VAL120 | CA | 75.52 | 33.525 | 42.13 |
| 942 | VAL120 | CB | 76.6 | 34.514 | 42.562 |
| 943 | VAL120 | CG1 | 77.342 | 35.091 | 41.364 |
| 944 | VAL120 | CG2 | 76.019 | 35.629 | 43.422 |
| 945 | VAL120 | C | 76.123 | 32.505 | 41.166 |
| 946 | VAL120 | O | 75.879 | 32.597 | 39.956 |
| 947 | GLU121 | N | 76.634 | 31.409 | 41.705 |
| 948 | GLU121 | CA | 77.197 | 30.354 | 40.86 |
| 949 | GLU121 | CB | 78.138 | 29.524 | 41.719 |
| 950 | GLU121 | CG | 79.338 | 30.365 | 42.136 |
| 951 | GLU121 | CD | 80.1 | 29.68 | 43.263 |
| 952 | GLU121 | OE1 | 79.445 | 29.111 | 44.125 |
| 953 | GLU121 | OE2 | 81.312 | 29.836 | 43.308 |
| 954 | GLU121 | C | 76.117 | 29.47 | 40.24 |
| 955 | GLU121 | O | 76.265 | 29.077 | 39.075 |
| 956 | TYR122 | N | 74.957 | 29.404 | 40.875 |
| 957 | TYR122 | CA | 73.83 | 28.679 | 40.286 |
| 958 | TYR122 | CB | 72.786 | 28.458 | 41.372 |
| 959 | TYR122 | CG | 71.555 | 27.664 | 40.941 |
| 960 | TYR122 | CD1 | 71.688 | 26.35 | 40.507 |
| 961 | TYR122 | CE1 | 70.563 | 25.625 | 40.132 |
| 962 | TYR122 | CZ | 69.308 | 26.214 | 40.198 |
| 963 | TYR122 | OH | 68.2 | 25.524 | 39.752 |
| 964 | TYR122 | CE2 | 69.172 | 27.524 | 40.64 |
| 965 | TYR122 | CD2 | 70.297 | 28.25 | 41.011 |
| 966 | TYR122 | C | 73.215 | 29.483 | 39.146 |
| 967 | TYR122 | O | 73.021 | 28.936 | 38.053 |
| 968 | VAL123 | N | 73.202 | 30.798 | 39.303 |
| 969 | VAL123 | CA | 72.686 | 31.678 | 38.249 |
| 970 | VAL123 | CB | 72.539 | 33.078 | 38.836 |
| 971 | VAL123 | CG1 | 72.183 | 34.096 | 37.763 |
| 972 | VAL123 | CG2 | 71.514 | 33.102 | 39.963 |
| 973 | VAL123 | C | 73.631 | 31.719 | 37.047 |
| 974 | VAL123 | O | 73.186 | 31.509 | 35.91 |
| 975 | LYS124 | N | 74.922 | 31.659 | 37.334 |
| 976 | LYS124 | CA | 75.947 | 31.652 | 36.285 |
| 977 | LYS124 | CB | 77.296 | 31.814 | 36.985 |
| 978 | LYS124 | CG | 78.472 | 31.835 | 36.014 |
| 979 | LYS124 | CD | 78.441 | 33.071 | 35.126 |
| 980 | LYS124 | CE | 79.599 | 33.084 | 34.134 |
| 981 | LYS124 | NZ | 79.54 | 34.272 | 33.267 |
| 982 | LYS124 | C | 75.946 | 30.348 | 35.485 |
| 983 | LYS124 | O | 76.015 | 30.403 | 34.25 |
| 984 | GLY125 | N | 75.588 | 29.255 | 36.144 |
| 985 | GLY125 | CA | 75.568 | 27.929 | 35.513 |
| 986 | GLY125 | C | 74.278 | 27.623 | 34.75 |
| 987 | GLY125 | O | 74.262 | 26.719 | 33.907 |
| 988 | LEU126 | N | 73.213 | 28.354 | 35.041 |
| 989 | LEU126 | CA | 71.959 | 28.181 | 34.297 |
| 990 | LEU126 | CB | 70.798 | 28.594 | 35.186 |
| 991 | LEU126 | CG | 70.643 | 27.665 | 36.378 |
| 992 | LEU126 | CD1 | 69.62 | 28.234 | 37.345 |
| 993 | LEU126 | CD2 | 70.258 | 26.255 | 35.943 |
| 994 | LEU126 | C | 71.92 | 29.033 | 33.034 |
| 995 | LEU126 | O | 70.995 | 28.902 | 32.223 |
| 996 | GLN127 | N | 72.896 | 29.913 | 32.9 |
| 997 | GLN127 | CA | 73.019 | 30.775 | 31.726 |
| 998 | GLN127 | CB | 74.011 | 31.846 | 32.13 |
| 999 | GLN127 | CG | 74.282 | 32.885 | 31.059 |
| 1000 | GLN127 | CD | 75.405 | 33.739 | 31.617 |
| 1001 | GLN127 | OE1 | 75.555 | 34.921 | 31.292 |
| 1002 | GLN127 | NE2 | 76.157 | 33.127 | 32.514 |
| 1003 | GLN127 | C | 73.565 | 30.008 | 30.528 |
| 1004 | GLN127 | O | 74.714 | 29.552 | 30.537 |
| 1005 | LYS128 | N | 72.753 | 29.908 | 29.493 |
| 1006 | LYS128 | CA | 73.155 | 29.176 | 28.29 |
| 1007 | LYS128 | CB | 71.918 | 28.602 | 27.62 |
| 1008 | LYS128 | CG | 71.157 | 27.714 | 28.593 |
| 1009 | LYS128 | CD | 71.968 | 26.515 | 29.07 |
| 1010 | LYS128 | CE | 71.18 | 25.72 | 30.106 |
| 1011 | LYS128 | NZ | 71.954 | 24.571 | 30.598 |
| 1012 | LYS128 | C | 73.903 | 30.069 | 27.313 |
| 1013 | LYS128 | O | 73.984 | 31.291 | 27.487 |
| 1014 | GLU129 | N | 74.282 | 29.473 | 26.194 |
| 1015 | GLU129 | CA | 75.105 | 30.16 | 25.184 |
| 1016 | GLU129 | CB | 75.707 | 29.129 | 24.225 |
| 1017 | GLU129 | CG | 76.667 | 28.148 | 24.899 |
| 1018 | GLU129 | CD | 76.027 | 26.768 | 25.051 |
| 1019 | GLU129 | OE1 | 74.834 | 26.729 | 25.333 |
| 1020 | GLU129 | OE2 | 76.744 | 25.786 | 24.937 |
| 1021 | GLU129 | C | 74.322 | 31.181 | 24.354 |
| 1022 | GLU129 | O | 74.92 | 31.99 | 23.639 |
| 1023 | ASP130 | N | 73.005 | 31.165 | 24.473 |
| 1024 | ASP130 | CA | 72.171 | 32.153 | 23.789 |
| 1025 | ASP130 | CB | 70.988 | 31.448 | 23.128 |
| 1026 | ASP130 | CG | 70.045 | 30.863 | 24.174 |
| 1027 | ASP130 | OD1 | 69.159 | 31.593 | 24.596 |
| 1028 | ASP130 | OD2 | 70.285 | 29.745 | 24.609 |
| 1029 | ASP130 | C | 71.678 | 33.239 | 24.75 |
| 1030 | ASP130 | O | 70.8 | 34.029 | 24.386 |
| 1031 | GLY131 | N | 72.13 | 33.195 | 25.995 |
| 1032 | GLY131 | CA | 71.702 | 34.194 | 26.98 |
| 1033 | GLY131 | C | 70.707 | 33.639 | 27.996 |
| 1034 | GLY131 | O | 70.881 | 33.824 | 29.207 |
| 1035 | SER132 | N | 69.681 | 32.974 | 27.483 |
| 1036 | SER132 | CA | 68.594 | 32.403 | 28.296 |
| 1037 | SER132 | CB | 67.827 | 31.415 | 27.436 |
| 1038 | SER132 | OG | 68.718 | 30.364 | 27.084 |
| 1039 | SER132 | C | 69.058 | 31.638 | 29.523 |
| 1040 | SER132 | O | 70.073 | 30.932 | 29.509 |
| 1041 | PHE133 | N | 68.308 | 31.82 | 30.594 |
| 1042 | PHE133 | CA | 68.565 | 31.067 | 31.815 |
| 1043 | PHE133 | CB | 68.489 | 31.997 | 33.02 |
| 1044 | PHE133 | CG | 69.614 | 33.025 | 33.105 |
| 1045 | PHE133 | CD1 | 69.51 | 34.246 | 32.45 |
| 1046 | PHE133 | CE1 | 70.539 | 35.173 | 32.533 |
| 1047 | PHE133 | CZ | 71.671 | 34.882 | 33.278 |
| 1048 | PHE133 | CE2 | 71.772 | 33.668 | 33.944 |
| 1049 | PHE133 | CD2 | 70.744 | 32.74 | 33.858 |
| 1050 | PHE133 | C | 67.566 | 29.931 | 31.966 |
| 1051 | PHE133 | O | 66.417 | 30.012 | 31.504 |
| 1052 | ALA134 | N | 68.096 | 28.813 | 32.425 |
| 1053 | ALA134 | CA | 67.276 | 27.655 | 32.771 |
| 1054 | ALA134 | CB | 68.122 | 26.395 | 32.631 |
| 1055 | ALA134 | C | 66.767 | 27.78 | 34.203 |
| 1056 | ALA134 | O | 67.438 | 28.355 | 35.065 |
| 1057 | GLY135 | N | 65.55 | 27.319 | 34.423 |
| 1058 | GLY135 | CA | 64.985 | 27.309 | 35.777 |
| 1059 | GLY135 | C | 65.741 | 26.306 | 36.633 |
| 1060 | GLY135 | O | 66.395 | 26.661 | 37.62 |
| 1061 | ASP136 | N | 65.503 | 25.045 | 36.341 |
| 1062 | ASP136 | CA | 66.294 | 23.968 | 36.927 |
| 1063 | ASP136 | CB | 65.384 | 22.791 | 37.279 |
| 1064 | ASP136 | CG | 64.51 | 22.357 | 36.1 |
| 1065 | ASP136 | OD1 | 65.055 | 22.194 | 35.012 |
| 1066 | ASP136 | OD2 | 63.361 | 22.029 | 36.349 |
| 1067 | ASP136 | C | 67.409 | 23.546 | 35.975 |
| 1068 | ASP136 | O | 67.361 | 23.826 | 34.765 |
| 1069 | ILE137 | N | 68.26 | 22.671 | 36.488 |
| 1070 | ILE137 | CA | 69.451 | 22.173 | 35.768 |
| 1071 | ILE137 | CB | 70.447 | 21.562 | 36.764 |
| 1072 | ILE137 | CG2 | 70.653 | 22.509 | 37.942 |
| 1073 | ILE137 | CG1 | 70.054 | 20.167 | 37.273 |
| 1074 | ILE137 | CD1 | 69.09 | 20.17 | 38.459 |
| 1075 | ILE137 | C | 69.173 | 21.138 | 34.667 |
| 1076 | ILE137 | O | 70.12 | 20.638 | 34.051 |
| 1077 | TRP138 | N | 67.908 | 20.886 | 34.36 |
| 1078 | TRP138 | CA | 67.547 | 19.932 | 33.313 |
| 1079 | TRP138 | CB | 66.201 | 19.314 | 33.679 |
| 1080 | TRP138 | CG | 66.215 | 18.583 | 35.01 |
| 1081 | TRP138 | CD1 | 65.637 | 18.992 | 36.193 |
| 1082 | TRP138 | NE1 | 65.888 | 18.055 | 37.143 |
| 1083 | TRP138 | CE2 | 66.607 | 17.034 | 36.639 |
| 1084 | TRP138 | CZ2 | 67.107 | 15.868 | 37.199 |
| 1085 | TRP138 | CH2 | 67.829 | 14.979 | 36.411 |
| 1086 | TRP138 | CZ3 | 68.055 | 15.253 | 35.067 |
| 1087 | TRP138 | CE3 | 67.56 | 16.42 | 34.498 |
| 1088 | TRP138 | CD2 | 66.84 | 17.31 | 35.279 |
| 1089 | TRP138 | C | 67.473 | 20.603 | 31.939 |
| 1090 | TRP138 | O | 67.276 | 19.923 | 30.925 |
| 1091 | GLY139 | N | 67.644 | 21.916 | 31.908 |
| 1092 | GLY139 | CA | 67.69 | 22.639 | 30.633 |
| 1093 | GLY139 | C | 66.352 | 23.299 | 30.342 |
| 1094 | GLY139 | O | 65.906 | 23.383 | 29.19 |
| 1095 | GLU140 | N | 65.754 | 23.826 | 31.395 |
| 1096 | GLU140 | CA | 64.424 | 24.442 | 31.31 |
| 1097 | GLU140 | CB | 63.816 | 24.274 | 32.693 |
| 1098 | GLU140 | CG | 62.367 | 24.724 | 32.806 |
| 1099 | GLU140 | CD | 62.053 | 24.741 | 34.292 |
| 1100 | GLU140 | OE1 | 63.021 | 24.737 | 35.041 |
| 1101 | GLU140 | OE2 | 60.89 | 24.746 | 34.66 |
| 1102 | GLU140 | C | 64.52 | 25.927 | 30.944 |
| 1103 | GLU140 | O | 64.366 | 26.798 | 31.809 |
| 1104 | ILE141 | N | 64.755 | 26.186 | 29.668 |
| 1105 | ILE141 | CA | 65.003 | 27.543 | 29.15 |
| 1106 | ILE141 | CB | 65.631 | 27.358 | 27.769 |
| 1107 | ILE141 | CG2 | 65.662 | 28.645 | 26.953 |
| 1108 | ILE141 | CG1 | 67.032 | 26.793 | 27.931 |
| 1109 | ILE141 | CD1 | 67.837 | 27.695 | 28.854 |
| 1110 | ILE141 | C | 63.744 | 28.396 | 29.044 |
| 1111 | ILE141 | O | 62.747 | 27.967 | 28.451 |
| 1112 | ASP142 | N | 63.791 | 29.588 | 29.625 |
| 1113 | ASP142 | CA | 62.645 | 30.501 | 29.515 |
| 1114 | ASP142 | CB | 61.535 | 29.924 | 30.394 |
| 1115 | ASP142 | CG | 60.164 | 30.46 | 30.003 |
| 1116 | ASP142 | OD1 | 59.82 | 31.521 | 30.513 |
| 1117 | ASP142 | OD2 | 59.499 | 29.829 | 29.198 |
| 1118 | ASP142 | C | 63.008 | 31.929 | 29.953 |
| 1119 | ASP142 | O | 63.785 | 32.125 | 30.898 |
| 1120 | THR143 | N | 62.321 | 32.912 | 29.383 |
| 1121 | THR143 | CA | 62.517 | 34.321 | 29.784 |
| 1122 | THR143 | CB | 61.731 | 35.245 | 28.858 |
| 1123 | THR143 | OG1 | 60.354 | 34.891 | 28.903 |
| 1124 | THR143 | CG2 | 62.199 | 35.159 | 27.418 |
| 1125 | THR143 | C | 62.066 | 34.637 | 31.212 |
| 1126 | THR143 | O | 62.637 | 35.541 | 31.827 |
| 1127 | ARG144 | N | 61.245 | 33.786 | 31.809 |
| 1128 | ARG144 | CA | 60.841 | 33.994 | 33.199 |
| 1129 | ARG144 | CB | 59.636 | 33.109 | 33.485 |
| 1130 | ARG144 | CG | 59.134 | 33.291 | 34.911 |
| 1131 | ARG144 | CD | 57.901 | 32.438 | 35.171 |
| 1132 | ARG144 | NE | 57.345 | 32.714 | 36.504 |
| 1133 | ARG144 | CZ | 56.78 | 31.775 | 37.265 |
| 1134 | ARG144 | NH1 | 56.761 | 30.506 | 36.852 |
| 1135 | ARG144 | NH2 | 56.272 | 32.098 | 38.456 |
| 1136 | ARG144 | C | 61.967 | 33.621 | 34.155 |
| 1137 | ARG144 | O | 62.222 | 34.359 | 35.111 |
| 1138 | PHE145 | N | 62.816 | 32.706 | 33.72 |
| 1139 | PHE145 | CA | 63.935 | 32.277 | 34.555 |
| 1140 | PHE145 | CB | 64.195 | 30.805 | 34.281 |
| 1141 | PHE145 | CG | 62.971 | 29.947 | 34.584 |
| 1142 | PHE145 | CD1 | 62.477 | 29.074 | 33.624 |
| 1143 | PHE145 | CE1 | 61.355 | 28.303 | 33.898 |
| 1144 | PHE145 | CZ | 60.726 | 28.406 | 35.132 |
| 1145 | PHE145 | CE2 | 61.22 | 29.278 | 36.093 |
| 1146 | PHE145 | CD2 | 62.342 | 30.048 | 35.82 |
| 1147 | PHE145 | C | 65.156 | 33.134 | 34.259 |
| 1148 | PHE145 | O | 65.986 | 33.369 | 35.144 |
| 1149 | SER146 | N | 65.095 | 33.825 | 33.134 |
| 1150 | SER146 | CA | 66.104 | 34.834 | 32.831 |
| 1151 | SER146 | CB | 66.066 | 35.125 | 31.334 |
| 1152 | SER146 | OG | 66.328 | 33.901 | 30.651 |
| 1153 | SER146 | C | 65.823 | 36.095 | 33.65 |
| 1154 | SER146 | O | 66.753 | 36.671 | 34.233 |
| 1155 | PHE147 | N | 64.548 | 36.328 | 33.922 |
| 1156 | PHE147 | CA | 64.134 | 37.407 | 34.824 |
| 1157 | PHE147 | CB | 62.643 | 37.65 | 34.619 |
| 1158 | PHE147 | CG | 61.99 | 38.534 | 35.677 |
| 1159 | PHE147 | CD1 | 62.496 | 39.799 | 35.949 |
| 1160 | PHE147 | CE1 | 61.897 | 40.593 | 36.917 |
| 1161 | PHE147 | CZ | 60.79 | 40.124 | 37.612 |
| 1162 | PHE147 | CE2 | 60.282 | 38.861 | 37.34 |
| 1163 | PHE147 | CD2 | 60.883 | 38.066 | 36.373 |
| 1164 | PHE147 | C | 64.399 | 37.052 | 36.286 |
| 1165 | PHE147 | O | 64.882 | 37.908 | 37.038 |
| 1166 | CYS148 | N | 64.312 | 35.775 | 36.62 |
| 1167 | CYS148 | CA | 64.647 | 35.343 | 37.979 |
| 1168 | CYS148 | CB | 64.276 | 33.875 | 38.157 |
| 1169 | CYS148 | SG | 62.513 | 33.488 | 38.089 |
| 1170 | CYS148 | C | 66.132 | 35.521 | 38.258 |
| 1171 | CYS148 | O | 66.481 | 36.178 | 39.245 |
| 1172 | ALA149 | N | 66.952 | 35.245 | 37.259 |
| 1173 | ALA149 | CA | 68.397 | 35.398 | 37.413 |
| 1174 | ALA149 | CB | 69.058 | 34.739 | 36.217 |
| 1175 | ALA149 | C | 68.842 | 36.856 | 37.481 |
| 1176 | ALA149 | O | 69.6 | 37.21 | 38.395 |
| 1177 | VAL150 | N | 68.197 | 37.721 | 36.712 |
| 1178 | VAL150 | CA | 68.599 | 39.132 | 36.723 |
| 1179 | VAL150 | CB | 68.159 | 39.802 | 35.415 |
| 1180 | VAL150 | CG1 | 66.648 | 39.961 | 35.306 |
| 1181 | VAL150 | CG2 | 68.816 | 41.163 | 35.232 |
| 1182 | VAL150 | C | 68.047 | 39.869 | 37.948 |
| 1183 | VAL150 | O | 68.749 | 40.732 | 38.488 |
| 1184 | ALA151 | N | 66.984 | 39.351 | 38.546 |
| 1185 | ALA151 | CA | 66.448 | 39.971 | 39.754 |
| 1186 | ALA151 | CB | 64.958 | 39.666 | 39.842 |
| 1187 | ALA151 | C | 67.169 | 39.454 | 40.992 |
| 1188 | ALA151 | O | 67.467 | 40.243 | 41.897 |
| 1189 | THR152 | N | 67.693 | 38.243 | 40.893 |
| 1190 | THR152 | CA | 68.463 | 37.669 | 41.996 |
| 1191 | THR152 | CB | 68.65 | 36.174 | 41.752 |
| 1192 | THR152 | OG1 | 67.378 | 35.548 | 41.833 |
| 1193 | THR152 | CG2 | 69.535 | 35.54 | 42.815 |
| 1194 | THR152 | C | 69.82 | 38.346 | 42.101 |
| 1195 | THR152 | O | 70.14 | 38.886 | 43.167 |
| 1196 | LEU153 | N | 70.448 | 38.595 | 40.962 |
| 1197 | LEU153 | CA | 71.746 | 39.27 | 40.993 |
| 1198 | LEU153 | CB | 72.504 | 38.977 | 39.71 |
| 1199 | LEU153 | CG | 72.987 | 37.535 | 39.663 |
| 1200 | LEU153 | CD1 | 73.843 | 37.316 | 38.425 |
| 1201 | LEU153 | CD2 | 73.79 | 37.195 | 40.914 |
| 1202 | LEU153 | C | 71.619 | 40.777 | 41.192 |
| 1203 | LEU153 | O | 72.527 | 41.387 | 41.772 |
| 1204 | ALA154 | N | 70.444 | 41.328 | 40.937 |
| 1205 | ALA154 | CA | 70.215 | 42.735 | 41.258 |
| 1206 | ALA154 | CB | 68.956 | 43.207 | 40.545 |
| 1207 | ALA154 | C | 70.05 | 42.926 | 42.763 |
| 1208 | ALA154 | O | 70.762 | 43.757 | 43.34 |
| 1209 | LEU155 | N | 69.379 | 41.983 | 43.41 |
| 1210 | LEU155 | CA | 69.156 | 42.067 | 44.862 |
| 1211 | LEU155 | CB | 67.967 | 41.186 | 45.223 |
| 1212 | LEU155 | CG | 66.673 | 41.709 | 44.616 |
| 1213 | LEU155 | CD1 | 65.531 | 40.726 | 44.838 |
| 1214 | LEU155 | CD2 | 66.325 | 43.08 | 45.179 |
| 1215 | LEU155 | C | 70.361 | 41.617 | 45.685 |
| 1216 | LEU155 | O | 70.443 | 41.931 | 46.877 |
| 1217 | LEU156 | N | 71.291 | 40.914 | 45.058 |
| 1218 | LEU156 | CA | 72.538 | 40.558 | 45.742 |
| 1219 | LEU156 | CB | 73.006 | 39.195 | 45.243 |
| 1220 | LEU156 | CG | 72.003 | 38.095 | 45.568 |
| 1221 | LEU156 | CD1 | 72.443 | 36.77 | 44.959 |
| 1222 | LEU156 | CD2 | 71.789 | 37.956 | 47.072 |
| 1223 | LEU156 | C | 73.642 | 41.586 | 45.497 |
| 1224 | LEU156 | O | 74.688 | 41.536 | 46.155 |
| 1225 | GLY157 | N | 73.406 | 42.508 | 44.576 |
| 1226 | GLY157 | CA | 74.401 | 43.533 | 44.247 |
| 1227 | GLY157 | C | 75.536 | 42.957 | 43.405 |
| 1228 | GLY157 | O | 76.683 | 43.412 | 43.487 |
| 1229 | LYS158 | N | 75.197 | 42.005 | 42.553 |
| 1230 | LYS158 | CA | 76.21 | 41.326 | 41.749 |
| 1231 | LYS158 | CB | 76.675 | 40.088 | 42.508 |
| 1232 | LYS158 | CG | 78.072 | 39.657 | 42.076 |
| 1233 | LYS158 | CD | 78.556 | 38.466 | 42.893 |
| 1234 | LYS158 | CE | 80.015 | 38.142 | 42.596 |
| 1235 | LYS158 | NZ | 80.219 | 37.876 | 41.164 |
| 1236 | LYS158 | C | 75.618 | 40.945 | 40.397 |
| 1237 | LYS158 | O | 75.796 | 39.824 | 39.9 |
| 1238 | LEU159 | N | 75.093 | 41.952 | 39.718 |
| 1239 | LEU159 | CA | 74.424 | 41.733 | 38.428 |
| 1240 | LEU159 | CB | 73.543 | 42.946 | 38.148 |
| 1241 | LEU159 | CG | 72.69 | 42.746 | 36.902 |
| 1242 | LEU159 | CD1 | 71.834 | 41.493 | 37.037 |
| 1243 | LEU159 | CD2 | 71.821 | 43.968 | 36.63 |
| 1244 | LEU159 | C | 75.42 | 41.531 | 37.283 |
| 1245 | LEU159 | O | 75.125 | 40.8 | 36.33 |
| 1246 | ASP160 | N | 76.668 | 41.886 | 37.547 |
| 1247 | ASP160 | CA | 77.757 | 41.757 | 36.571 |
| 1248 | ASP160 | CB | 78.823 | 42.8 | 36.892 |
| 1249 | ASP160 | CG | 78.221 | 44.203 | 36.873 |
| 1250 | ASP160 | OD1 | 78.047 | 44.733 | 35.786 |
| 1251 | ASP160 | OD2 | 77.842 | 44.67 | 37.94 |
| 1252 | ASP160 | C | 78.404 | 40.368 | 36.573 |
| 1253 | ASP160 | O | 79.493 | 40.199 | 36.014 |
| 1254 | ALA161 | N | 77.787 | 39.411 | 37.252 |
| 1255 | ALA161 | CA | 78.308 | 38.044 | 37.271 |
| 1256 | ALA161 | CB | 77.81 | 37.358 | 38.535 |
| 1257 | ALA161 | C | 77.835 | 37.248 | 36.058 |
| 1258 | ALA161 | O | 78.38 | 36.179 | 35.764 |
| 1259 | ILE162 | N | 76.823 | 37.758 | 35.375 |
| 1260 | ILE162 | CA | 76.36 | 37.14 | 34.131 |
| 1261 | ILE162 | CB | 74.865 | 36.878 | 34.241 |
| 1262 | ILE162 | CG2 | 74.595 | 35.761 | 35.243 |
| 1263 | ILE162 | CG1 | 74.131 | 38.16 | 34.626 |
| 1264 | ILE162 | CD1 | 72.626 | 37.949 | 34.743 |
| 1265 | ILE162 | C | 76.636 | 38.061 | 32.949 |
| 1266 | ILE162 | O | 76.975 | 39.238 | 33.124 |
| 1267 | ASN163 | N | 76.533 | 37.51 | 31.753 |
| 1268 | ASN163 | CA | 76.664 | 38.33 | 30.556 |
| 1269 | ASN163 | CB | 77.185 | 37.504 | 29.387 |
| 1270 | ASN163 | CG | 77.52 | 38.44 | 28.227 |
| 1271 | ASN163 | OD1 | 76.636 | 39.092 | 27.656 |
| 1272 | ASN163 | ND2 | 78.804 | 38.569 | 27.95 |
| 1273 | ASN163 | C | 75.295 | 38.909 | 30.235 |
| 1274 | ASN163 | O | 74.505 | 38.347 | 29.462 |
| 1275 | VAL164 | N | 75.138 | 40.152 | 30.651 |
| 1276 | VAL164 | CA | 73.85 | 40.831 | 30.551 |
| 1277 | VAL164 | CB | 73.94 | 42.094 | 31.404 |
| 1278 | VAL164 | CG1 | 72.615 | 42.845 | 31.43 |
| 1279 | VAL164 | CG2 | 74.381 | 41.757 | 32.825 |
| 1280 | VAL164 | C | 73.486 | 41.185 | 29.109 |
| 1281 | VAL164 | O | 72.321 | 40.999 | 28.746 |
| 1282 | GLU165 | N | 74.481 | 41.284 | 28.241 |
| 1283 | GLU165 | CA | 74.223 | 41.656 | 26.848 |
| 1284 | GLU165 | CB | 75.555 | 42.062 | 26.228 |
| 1285 | GLU165 | CG | 75.417 | 42.42 | 24.753 |
| 1286 | GLU165 | CD | 76.8 | 42.695 | 24.171 |
| 1287 | GLU165 | OE1 | 77.755 | 42.154 | 24.714 |
| 1288 | GLU165 | OE2 | 76.885 | 43.473 | 23.232 |
| 1289 | GLU165 | C | 73.636 | 40.492 | 26.051 |
| 1290 | GLU165 | O | 72.663 | 40.686 | 25.312 |
| 1291 | LYS166 | N | 74.033 | 39.282 | 26.408 |
| 1292 | LYS166 | CA | 73.547 | 38.102 | 25.699 |
| 1293 | LYS166 | CB | 74.549 | 36.975 | 25.919 |
| 1294 | LYS166 | CG | 74.45 | 35.928 | 24.818 |
| 1295 | LYS166 | CD | 74.854 | 36.531 | 23.478 |
| 1296 | LYS166 | CE | 74.732 | 35.522 | 22.343 |
| 1297 | LYS166 | NZ | 73.333 | 35.112 | 22.156 |
| 1298 | LYS166 | C | 72.179 | 37.688 | 26.229 |
| 1299 | LYS166 | O | 71.309 | 37.285 | 25.447 |
| 1300 | ALA167 | N | 71.914 | 38.042 | 27.477 |
| 1301 | ALA167 | CA | 70.606 | 37.754 | 28.066 |
| 1302 | ALA167 | CB | 70.746 | 37.784 | 29.582 |
| 1303 | ALA167 | C | 69.564 | 38.772 | 27.603 |
| 1304 | ALA167 | O | 68.433 | 38.385 | 27.278 |
| 1305 | ILE168 | N | 70.023 | 39.978 | 27.304 |
| 1306 | ILE168 | CA | 69.148 | 40.998 | 26.72 |
| 1307 | ILE168 | CB | 69.83 | 42.358 | 26.837 |
| 1308 | ILE168 | CG2 | 69.078 | 43.419 | 26.046 |
| 1309 | ILE168 | CG1 | 69.956 | 42.793 | 28.29 |
| 1310 | ILE168 | CD1 | 70.807 | 44.052 | 28.402 |
| 1311 | ILE168 | C | 68.877 | 40.691 | 25.252 |
| 1312 | ILE168 | O | 67.725 | 40.801 | 24.819 |
| 1313 | GLU169 | N | 69.822 | 40.029 | 24.603 |
| 1314 | GLU169 | CA | 69.627 | 39.609 | 23.214 |
| 1315 | GLU169 | CB | 70.976 | 39.156 | 22.673 |
| 1316 | GLU169 | CG | 70.889 | 38.711 | 21.219 |
| 1317 | GLU169 | CD | 72.274 | 38.297 | 20.739 |
| 1318 | GLU169 | OE1 | 73.239 | 38.76 | 21.333 |
| 1319 | GLU169 | OE2 | 72.347 | 37.508 | 19.807 |
| 1320 | GLU169 | C | 68.614 | 38.468 | 23.107 |
| 1321 | GLU169 | O | 67.734 | 38.523 | 22.237 |
| 1322 | PHE170 | N | 68.572 | 37.61 | 24.114 |
| 1323 | PHE170 | CA | 67.557 | 36.557 | 24.134 |
| 1324 | PHE170 | CB | 67.912 | 35.534 | 25.204 |
| 1325 | PHE170 | CG | 66.845 | 34.457 | 25.376 |
| 1326 | PHE170 | CD1 | 66.655 | 33.505 | 24.383 |
| 1327 | PHE170 | CE1 | 65.681 | 32.527 | 24.535 |
| 1328 | PHE170 | CZ | 64.891 | 32.504 | 25.676 |
| 1329 | PHE170 | CE2 | 65.075 | 33.46 | 26.666 |
| 1330 | PHE170 | CD2 | 66.05 | 34.438 | 26.516 |
| 1331 | PHE170 | C | 66.171 | 37.122 | 24.427 |
| 1332 | PHE170 | O | 65.223 | 36.796 | 23.706 |
| 1333 | VAL171 | N | 66.095 | 38.125 | 25.285 |
| 1334 | VAL171 | CA | 64.789 | 38.709 | 25.6 |
| 1335 | VAL171 | CB | 64.921 | 39.516 | 26.887 |
| 1336 | VAL171 | CG1 | 63.66 | 40.321 | 27.181 |
| 1337 | VAL171 | CG2 | 65.25 | 38.594 | 28.054 |
| 1338 | VAL171 | C | 64.256 | 39.581 | 24.463 |
| 1339 | VAL171 | O | 63.072 | 39.459 | 24.121 |
| 1340 | LEU172 | N | 65.15 | 40.184 | 23.695 |
| 1341 | LEU172 | CA | 64.711 | 41 | 22.558 |
| 1342 | LEU172 | CB | 65.819 | 41.97 | 22.173 |
| 1343 | LEU172 | CG | 66.098 | 42.971 | 23.286 |
| 1344 | LEU172 | CD1 | 67.26 | 43.881 | 22.907 |
| 1345 | LEU172 | CD2 | 64.854 | 43.785 | 23.623 |
| 1346 | LEU172 | C | 64.339 | 40.151 | 21.347 |
| 1347 | LEU172 | O | 63.425 | 40.532 | 20.605 |
| 1348 | SER173 | N | 64.838 | 38.925 | 21.293 |
| 1349 | SER173 | CA | 64.444 | 38.006 | 20.218 |
| 1350 | SER173 | CB | 65.569 | 37.016 | 19.936 |
| 1351 | SER173 | OG | 65.713 | 36.164 | 21.062 |
| 1352 | SER173 | C | 63.156 | 37.249 | 20.559 |
| 1353 | SER173 | O | 62.683 | 36.438 | 19.755 |
| 1354 | CYS174 | N | 62.611 | 37.493 | 21.741 |
| 1355 | CYS174 | CA | 61.299 | 36.955 | 22.098 |
| 1356 | CYS174 | CB | 61.309 | 36.569 | 23.569 |
| 1357 | CYS174 | SG | 62.54 | 35.332 | 24.02 |
| 1358 | CYS174 | C | 60.183 | 37.971 | 21.86 |
| 1359 | CYS174 | O | 59.009 | 37.626 | 22.047 |
| 1360 | MET175 | N | 60.534 | 39.18 | 21.442 |
| 1361 | MET175 | CA | 59.533 | 40.231 | 21.211 |
| 1362 | MET175 | CB | 60.266 | 41.546 | 20.948 |
| 1363 | MET175 | CG | 59.313 | 42.736 | 20.87 |
| 1364 | MET175 | SD | 60.063 | 44.323 | 20.436 |
| 1365 | MET175 | CE | 61.269 | 44.459 | 21.774 |
| 1366 | MET175 | C | 58.637 | 39.897 | 20.019 |
| 1367 | MET175 | O | 59.108 | 39.523 | 18.939 |
| 1368 | ASN176 | N | 57.34 | 39.993 | 20.247 |
| 1369 | ASN176 | CA | 56.355 | 39.748 | 19.197 |
| 1370 | ASN176 | CB | 55.116 | 39.118 | 19.814 |
| 1371 | ASN176 | CG | 55.467 | 37.787 | 20.466 |
| 1372 | ASN176 | OD1 | 55.577 | 37.69 | 21.691 |
| 1373 | ASN176 | ND2 | 55.604 | 36.767 | 19.641 |
| 1374 | ASN176 | C | 55.968 | 41.045 | 18.503 |
| 1375 | ASN176 | O | 56.294 | 42.148 | 18.959 |
| 1376 | PHE177 | N | 55.075 | 40.902 | 17.537 |
| 1377 | PHE177 | CA | 54.623 | 42.033 | 16.707 |
| 1378 | PHE177 | CB | 54.004 | 41.477 | 15.42 |
| 1379 | PHE177 | CG | 52.796 | 40.55 | 15.594 |
| 1380 | PHE177 | CD1 | 51.516 | 41.084 | 15.682 |
| 1381 | PHE177 | CE1 | 50.42 | 40.246 | 15.84 |
| 1382 | PHE177 | CZ | 50.6 | 38.871 | 15.9 |
| 1383 | PHE177 | CE2 | 51.876 | 38.333 | 15.794 |
| 1384 | PHE177 | CD2 | 52.972 | 39.171 | 15.636 |
| 1385 | PHE177 | C | 53.619 | 42.956 | 17.41 |
| 1386 | PHE177 | O | 53.224 | 43.985 | 16.856 |
| 1387 | ASP178 | N | 53.227 | 42.597 | 18.622 |
| 1388 | ASP178 | CA | 52.327 | 43.431 | 19.418 |
| 1389 | ASP178 | CB | 51.263 | 42.542 | 20.058 |
| 1390 | ASP178 | CG | 51.885 | 41.492 | 20.978 |
| 1391 | ASP178 | OD1 | 52.14 | 41.822 | 22.128 |
| 1392 | ASP178 | OD2 | 52.139 | 40.394 | 20.5 |
| 1393 | ASP178 | C | 53.082 | 44.215 | 20.495 |
| 1394 | ASP178 | O | 52.456 | 44.865 | 21.339 |
| 1395 | GLY179 | N | 54.4 | 44.084 | 20.535 |
| 1396 | GLY179 | CA | 55.183 | 44.806 | 21.545 |
| 1397 | GLY179 | C | 55.624 | 43.891 | 22.687 |
| 1398 | GLY179 | O | 56.785 | 43.926 | 23.112 |
| 1399 | GLY180 | N | 54.685 | 43.104 | 23.187 |
| 1400 | GLY180 | CA | 54.954 | 42.16 | 24.276 |
| 1401 | GLY180 | C | 55.894 | 41.035 | 23.866 |
| 1402 | GLY180 | O | 56.266 | 40.899 | 22.695 |
| 1403 | PHE181 | N | 56.258 | 40.23 | 24.847 |
| 1404 | PHE181 | CA | 57.252 | 39.176 | 24.641 |
| 1405 | PHE181 | CB | 58.408 | 39.422 | 25.609 |
| 1406 | PHE181 | CG | 59.151 | 40.756 | 25.478 |
| 1407 | PHE181 | CD1 | 58.71 | 41.886 | 26.16 |
| 1408 | PHE181 | CE1 | 59.397 | 43.086 | 26.04 |
| 1409 | PHE181 | CZ | 60.536 | 43.157 | 25.249 |
| 1410 | PHE181 | CE2 | 60.987 | 42.026 | 24.581 |
| 1411 | PHE181 | CD2 | 60.297 | 40.826 | 24.7 |
| 1412 | PHE181 | C | 56.675 | 37.789 | 24.918 |
| 1413 | PHE181 | O | 55.765 | 37.633 | 25.747 |
| 1414 | GLY182 | N | 57.208 | 36.805 | 24.213 |
| 1415 | GLY182 | CA | 56.882 | 35.4 | 24.477 |
| 1416 | GLY182 | C | 57.832 | 34.795 | 25.512 |
| 1417 | GLY182 | O | 58.746 | 35.461 | 26.017 |
| 1418 | CYS183 | N | 57.596 | 33.535 | 25.843 |
| 1419 | CYS183 | CA | 58.412 | 32.872 | 26.873 |
| 1420 | CYS183 | CB | 57.593 | 31.755 | 27.521 |
| 1421 | CYS183 | SG | 56.923 | 30.465 | 26.445 |
| 1422 | CYS183 | C | 59.721 | 32.336 | 26.303 |
| 1423 | CYS183 | O | 60.746 | 32.285 | 26.999 |
| 1424 | ARG184 | N | 59.661 | 31.987 | 25.029 |
| 1425 | ARG184 | CA | 60.821 | 31.649 | 24.203 |
| 1426 | ARG184 | CB | 60.893 | 30.131 | 24.047 |
| 1427 | ARG184 | CG | 61.178 | 29.422 | 25.366 |
| 1428 | ARG184 | CD | 61.162 | 27.911 | 25.182 |
| 1429 | ARG184 | NE | 59.858 | 27.476 | 24.657 |
| 1430 | ARG184 | CZ | 59.717 | 26.79 | 23.52 |
| 1431 | ARG184 | NH1 | 60.792 | 26.458 | 22.802 |
| 1432 | ARG184 | NH2 | 58.499 | 26.439 | 23.1 |
| 1433 | ARG184 | C | 60.573 | 32.309 | 22.851 |
| 1434 | ARG184 | O | 59.416 | 32.655 | 22.578 |
| 1435 | PRO185 | N | 61.602 | 32.503 | 22.037 |
| 1436 | PRO185 | CA | 61.419 | 33.187 | 20.751 |
| 1437 | PRO185 | CB | 62.78 | 33.235 | 20.127 |
| 1438 | PRO185 | CG | 63.788 | 32.59 | 21.064 |
| 1439 | PRO185 | CD | 62.999 | 32.133 | 22.28 |
| 1440 | PRO185 | C | 60.422 | 32.446 | 19.868 |
| 1441 | PRO185 | O | 60.53 | 31.231 | 19.667 |
| 1442 | GLY186 | N | 59.375 | 33.156 | 19.482 |
| 1443 | GLY186 | CA | 58.321 | 32.557 | 18.66 |
| 1444 | GLY186 | C | 57.001 | 32.432 | 19.422 |
| 1445 | GLY186 | O | 55.924 | 32.504 | 18.818 |
| 1446 | SER187 | N | 57.092 | 32.285 | 20.736 |
| 1447 | SER187 | CA | 55.898 | 32.139 | 21.582 |
| 1448 | SER187 | CB | 56.326 | 31.784 | 22.998 |
| 1449 | SER187 | OG | 57.157 | 30.632 | 22.943 |
| 1450 | SER187 | C | 55.118 | 33.445 | 21.608 |
| 1451 | SER187 | O | 55.683 | 34.502 | 21.314 |
| 1452 | GLU188 | N | 53.83 | 33.358 | 21.888 |
| 1453 | GLU188 | CA | 52.959 | 34.543 | 21.886 |
| 1454 | GLU188 | CB | 51.515 | 34.073 | 21.752 |
| 1455 | GLU188 | CG | 51.31 | 33.256 | 20.481 |
| 1456 | GLU188 | CD | 49.86 | 32.788 | 20.388 |
| 1457 | GLU188 | OE1 | 49 | 33.514 | 20.867 |
| 1458 | GLU188 | OE2 | 49.646 | 31.7 | 19.874 |
| 1459 | GLU188 | C | 53.099 | 35.377 | 23.159 |
| 1460 | GLU188 | O | 53.511 | 34.866 | 24.207 |
| 1461 | SER189 | N | 52.781 | 36.656 | 23.031 |
| 1462 | SER189 | CA | 52.765 | 37.579 | 24.175 |
| 1463 | SER189 | CB | 52.602 | 39.008 | 23.67 |
| 1464 | SER189 | OG | 53.678 | 39.334 | 22.807 |
| 1465 | SER189 | C | 51.591 | 37.318 | 25.108 |
| 1466 | SER189 | O | 50.468 | 37.041 | 24.667 |
| 1467 | HIS190 | N | 51.866 | 37.434 | 26.395 |
| 1468 | HIS190 | CA | 50.805 | 37.41 | 27.413 |
| 1469 | HIS190 | CB | 50.353 | 35.98 | 27.709 |
| 1470 | HIS190 | CG | 51.355 | 35.073 | 28.396 |
| 1471 | HIS190 | ND1 | 51.303 | 34.665 | 29.679 |
| 1472 | HIS190 | CE1 | 52.36 | 33.866 | 29.929 |
| 1473 | HIS190 | NE2 | 53.068 | 33.745 | 28.784 |
| 1474 | HIS190 | CD2 | 52.453 | 34.473 | 27.826 |
| 1475 | HIS190 | C | 51.286 | 38.116 | 28.677 |
| 1476 | HIS190 | O | 52.497 | 38.204 | 28.914 |
| 1477 | ALA191 | N | 50.343 | 38.517 | 29.516 |
| 1478 | ALA191 | CA | 50.613 | 39.311 | 30.735 |
| 1479 | ALA191 | CB | 49.337 | 39.332 | 31.565 |
| 1480 | ALA191 | C | 51.748 | 38.813 | 31.631 |
| 1481 | ALA191 | O | 52.654 | 39.592 | 31.948 |
| 1482 | GLY192 | N | 51.797 | 37.512 | 31.876 |
| 1483 | GLY192 | CA | 52.849 | 36.921 | 32.714 |
| 1484 | GLY192 | C | 54.245 | 37.158 | 32.145 |
| 1485 | GLY192 | O | 55.092 | 37.772 | 32.806 |
| 1486 | GLN193 | N | 54.386 | 36.907 | 30.855 |
| 1487 | GLN193 | CA | 55.689 | 37.027 | 30.208 |
| 1488 | GLN193 | CB | 55.622 | 36.276 | 28.895 |
| 1489 | GLN193 | CG | 56.84 | 35.387 | 28.781 |
| 1490 | GLN193 | CD | 56.857 | 34.42 | 29.956 |
| 1491 | GLN193 | OE1 | 55.811 | 33.94 | 30.408 |
| 1492 | GLN193 | NE2 | 58.058 | 34.043 | 30.347 |
| 1493 | GLN193 | C | 56.074 | 38.466 | 29.92 |
| 1494 | GLN193 | O | 57.258 | 38.814 | 30.005 |
| 1495 | ILE194 | N | 55.079 | 39.327 | 29.816 |
| 1496 | ILE194 | CA | 55.361 | 40.743 | 29.636 |
| 1497 | ILE194 | CB | 54.12 | 41.417 | 29.075 |
| 1498 | ILE194 | CG2 | 54.309 | 42.927 | 28.988 |
| 1499 | ILE194 | CG1 | 53.811 | 40.838 | 27.703 |
| 1500 | ILE194 | CD1 | 52.583 | 41.491 | 27.091 |
| 1501 | ILE194 | C | 55.788 | 41.367 | 30.957 |
| 1502 | ILE194 | O | 56.769 | 42.116 | 30.954 |
| 1503 | TYR195 | N | 55.318 | 40.815 | 32.064 |
| 1504 | TYR195 | CA | 55.789 | 41.27 | 33.372 |
| 1505 | TYR195 | CB | 54.917 | 40.672 | 34.47 |
| 1506 | TYR195 | CG | 55.355 | 41.078 | 35.875 |
| 1507 | TYR195 | CD1 | 54.944 | 42.3 | 36.389 |
| 1508 | TYR195 | CE1 | 55.35 | 42.688 | 37.658 |
| 1509 | TYR195 | CZ | 56.166 | 41.856 | 38.411 |
| 1510 | TYR195 | OH | 56.679 | 42.313 | 39.607 |
| 1511 | TYR195 | CE2 | 56.563 | 40.625 | 37.909 |
| 1512 | TYR195 | CD2 | 56.154 | 40.235 | 36.64 |
| 1513 | TYR195 | C | 57.23 | 40.842 | 33.598 |
| 1514 | TYR195 | O | 58.074 | 41.695 | 33.904 |
| 1515 | CYS196 | N | 57.545 | 39.625 | 33.188 |
| 1516 | CYS196 | CA | 58.899 | 39.102 | 33.375 |
| 1517 | CYS196 | CB | 58.895 | 37.619 | 33.025 |
| 1518 | CYS196 | SG | 57.805 | 36.595 | 34.037 |
| 1519 | CYS196 | C | 59.924 | 39.822 | 32.506 |
| 1520 | CYS196 | O | 60.941 | 40.29 | 33.032 |
| 1521 | CYS197 | N | 59.558 | 40.132 | 31.275 |
| 1522 | CYS197 | CA | 60.518 | 40.777 | 30.382 |
| 1523 | CYS197 | CB | 60.163 | 40.402 | 28.955 |
| 1524 | CYS197 | SG | 60.243 | 38.631 | 28.604 |
| 1525 | CYS197 | C | 60.584 | 42.295 | 30.547 |
| 1526 | CYS197 | O | 61.663 | 42.864 | 30.343 |
| 1527 | THR198 | N | 59.554 | 42.909 | 31.11 |
| 1528 | THR198 | CA | 59.662 | 44.339 | 31.428 |
| 1529 | THR198 | CB | 58.291 | 45.012 | 31.494 |
| 1530 | THR198 | OG1 | 57.483 | 44.352 | 32.463 |
| 1531 | THR198 | CG2 | 57.573 | 44.989 | 30.149 |
| 1532 | THR198 | C | 60.393 | 44.525 | 32.751 |
| 1533 | THR198 | O | 61.157 | 45.486 | 32.895 |
| 1534 | GLY199 | N | 60.334 | 43.512 | 33.601 |
| 1535 | GLY199 | CA | 61.138 | 43.491 | 34.818 |
| 1536 | GLY199 | C | 62.611 | 43.372 | 34.452 |
| 1537 | GLY199 | O | 63.409 | 44.254 | 34.795 |
| 1538 | PHE200 | N | 62.901 | 42.417 | 33.581 |
| 1539 | PHE200 | CA | 64.263 | 42.179 | 33.092 |
| 1540 | PHE200 | CB | 64.189 | 41.043 | 32.071 |
| 1541 | PHE200 | CG | 65.533 | 40.527 | 31.557 |
| 1542 | PHE200 | CD1 | 66.034 | 39.326 | 32.039 |
| 1543 | PHE200 | CE1 | 67.257 | 38.85 | 31.587 |
| 1544 | PHE200 | CZ | 67.973 | 39.569 | 30.641 |
| 1545 | PHE200 | CE2 | 67.462 | 40.757 | 30.138 |
| 1546 | PHE200 | CD2 | 66.239 | 41.231 | 30.59 |
| 1547 | PHE200 | C | 64.849 | 43.42 | 32.421 |
| 1548 | PHE200 | O | 65.894 | 43.915 | 32.863 |
| 1549 | LEU201 | N | 64.072 | 44.05 | 31.554 |
| 1550 | LEU201 | CA | 64.576 | 45.213 | 30.82 |
| 1551 | LEU201 | CB | 63.682 | 45.451 | 29.605 |
| 1552 | LEU201 | CG | 64.394 | 45.125 | 28.29 |
| 1553 | LEU201 | CD1 | 65.075 | 43.762 | 28.297 |
| 1554 | LEU201 | CD2 | 63.449 | 45.237 | 27.101 |
| 1555 | LEU201 | C | 64.661 | 46.473 | 31.681 |
| 1556 | LEU201 | O | 65.585 | 47.266 | 31.465 |
| 1557 | ALA202 | N | 63.933 | 46.52 | 32.785 |
| 1558 | ALA202 | CA | 64.053 | 47.656 | 33.702 |
| 1559 | ALA202 | CB | 62.767 | 47.785 | 34.508 |
| 1560 | ALA202 | C | 65.242 | 47.516 | 34.648 |
| 1561 | ALA202 | O | 65.863 | 48.526 | 35.006 |
| 1562 | ILE203 | N | 65.669 | 46.286 | 34.887 |
| 1563 | ILE203 | CA | 66.85 | 46.049 | 35.726 |
| 1564 | ILE203 | CB | 66.762 | 44.63 | 36.281 |
| 1565 | ILE203 | CG2 | 67.979 | 44.3 | 37.136 |
| 1566 | ILE203 | CG1 | 65.493 | 44.438 | 37.097 |
| 1567 | ILE203 | CD1 | 65.345 | 42.987 | 37.536 |
| 1568 | ILE203 | C | 68.131 | 46.187 | 34.908 |
| 1569 | ILE203 | O | 69.156 | 46.662 | 35.411 |
| 1570 | THR204 | N | 68.017 | 45.915 | 33.619 |
| 1571 | THR204 | CA | 69.161 | 46.061 | 32.708 |
| 1572 | THR204 | CB | 69.03 | 45.034 | 31.592 |
| 1573 | THR204 | OG1 | 67.834 | 45.31 | 30.873 |
| 1574 | THR204 | CG2 | 68.96 | 43.614 | 32.139 |
| 1575 | THR204 | C | 69.258 | 47.449 | 32.076 |
| 1576 | THR204 | O | 70.16 | 47.681 | 31.263 |
| 1577 | SER205 | N | 68.301 | 48.315 | 32.386 |
| 1578 | SER205 | CA | 68.222 | 49.684 | 31.845 |
| 1579 | SER205 | CB | 69.455 | 50.464 | 32.281 |
| 1580 | SER205 | OG | 69.513 | 50.396 | 33.699 |
| 1581 | SER205 | C | 68.081 | 49.72 | 30.321 |
| 1582 | SER205 | O | 68.427 | 50.718 | 29.677 |
| 1583 | GLN206 | N | 67.332 | 48.758 | 29.809 |
| 1584 | GLN206 | CA | 67.07 | 48.622 | 28.374 |
| 1585 | GLN206 | CB | 67.266 | 47.17 | 27.965 |
| 1586 | GLN206 | CG | 68.734 | 46.823 | 27.777 |
| 1587 | GLN206 | CD | 69.254 | 47.459 | 26.491 |
| 1588 | GLN206 | OE1 | 70.358 | 48.013 | 26.459 |
| 1589 | GLN206 | NE2 | 68.47 | 47.32 | 25.434 |
| 1590 | GLN206 | C | 65.651 | 49.046 | 28.045 |
| 1591 | GLN206 | O | 65.029 | 48.528 | 27.107 |
| 1592 | LEU207 | N | 65.228 | 50.118 | 28.694 |
| 1593 | LEU207 | CA | 63.839 | 50.588 | 28.601 |
| 1594 | LEU207 | CB | 63.534 | 51.554 | 29.748 |
| 1595 | LEU207 | CG | 63.26 | 50.884 | 31.096 |
| 1596 | LEU207 | CD1 | 62.333 | 49.683 | 30.931 |
| 1597 | LEU207 | CD2 | 64.533 | 50.49 | 31.839 |
| 1598 | LEU207 | C | 63.575 | 51.304 | 27.282 |
| 1599 | LEU207 | O | 62.455 | 51.248 | 26.765 |
| 1600 | HIS208 | N | 64.659 | 51.681 | 26.624 |
| 1601 | HIS208 | CA | 64.615 | 52.335 | 25.316 |
| 1602 | HIS208 | CB | 65.927 | 53.101 | 25.145 |
| 1603 | HIS208 | CG | 67.18 | 52.29 | 25.439 |
| 1604 | HIS208 | ND1 | 67.955 | 52.373 | 26.54 |
| 1605 | HIS208 | CE1 | 68.969 | 51.489 | 26.437 |
| 1606 | HIS208 | NE2 | 68.845 | 50.859 | 25.248 |
| 1607 | HIS208 | CD2 | 67.756 | 51.348 | 24.617 |
| 1608 | HIS208 | C | 64.434 | 51.341 | 24.163 |
| 1609 | HIS208 | O | 64.251 | 51.753 | 23.013 |
| 1610 | GLN209 | N | 64.453 | 50.052 | 24.473 |
| 1611 | GLN209 | CA | 64.23 | 49.034 | 23.452 |
| 1612 | GLN209 | CB | 65.088 | 47.824 | 23.796 |
| 1613 | GLN209 | CG | 65.65 | 47.163 | 22.544 |
| 1614 | GLN209 | CD | 66.873 | 47.937 | 22.066 |
| 1615 | GLN209 | OE1 | 67.452 | 48.721 | 22.829 |
| 1616 | GLN209 | NE2 | 67.355 | 47.574 | 20.89 |
| 1617 | GLN209 | C | 62.763 | 48.605 | 23.435 |
| 1618 | GLN209 | O | 62.319 | 47.918 | 22.506 |
| 1619 | VAL210 | N | 62.021 | 49.022 | 24.45 |
| 1620 | VAL210 | CA | 60.607 | 48.656 | 24.557 |
| 1621 | VAL210 | CB | 60.213 | 48.716 | 26.031 |
| 1622 | VAL210 | CG1 | 58.783 | 48.233 | 26.249 |
| 1623 | VAL210 | CG2 | 61.174 | 47.912 | 26.894 |
| 1624 | VAL210 | C | 59.73 | 49.633 | 23.781 |
| 1625 | VAL210 | O | 59.797 | 50.848 | 24.002 |
| 1626 | ASN211 | N | 58.915 | 49.109 | 22.879 |
| 1627 | ASN211 | CA | 57.911 | 49.961 | 22.237 |
| 1628 | ASN211 | CB | 57.444 | 49.342 | 20.922 |
| 1629 | ASN211 | CG | 56.58 | 50.333 | 20.136 |
| 1630 | ASN211 | OD1 | 55.705 | 51.015 | 20.689 |
| 1631 | ASN211 | ND2 | 56.819 | 50.378 | 18.839 |
| 1632 | ASN211 | C | 56.735 | 50.119 | 23.194 |
| 1633 | ASN211 | O | 55.725 | 49.409 | 23.091 |
| 1634 | SER212 | N | 56.769 | 51.223 | 23.922 |
| 1635 | SER212 | CA | 55.784 | 51.476 | 24.972 |
| 1636 | SER212 | CB | 56.35 | 52.544 | 25.898 |
| 1637 | SER212 | OG | 57.55 | 52.041 | 26.472 |
| 1638 | SER212 | C | 54.434 | 51.936 | 24.433 |
| 1639 | SER212 | O | 53.422 | 51.724 | 25.103 |
| 1640 | ASP213 | N | 54.369 | 52.304 | 23.167 |
| 1641 | ASP213 | CA | 53.09 | 52.729 | 22.603 |
| 1642 | ASP213 | CB | 53.356 | 53.63 | 21.401 |
| 1643 | ASP213 | CG | 54.158 | 54.857 | 21.825 |
| 1644 | ASP213 | OD1 | 53.543 | 55.798 | 22.305 |
| 1645 | ASP213 | OD2 | 55.376 | 54.818 | 21.7 |
| 1646 | ASP213 | C | 52.282 | 51.516 | 22.159 |
| 1647 | ASP213 | O | 51.122 | 51.364 | 22.564 |
| 1648 | LEU214 | N | 52.973 | 50.544 | 21.586 |
| 1649 | LEU214 | CA | 52.295 | 49.353 | 21.072 |
| 1650 | LEU214 | CB | 53.175 | 48.751 | 19.985 |
| 1651 | LEU214 | CG | 52.498 | 47.59 | 19.269 |
| 1652 | LEU214 | CD1 | 51.138 | 48.001 | 18.715 |
| 1653 | LEU214 | CD2 | 53.394 | 47.056 | 18.158 |
| 1654 | LEU214 | C | 52.052 | 48.339 | 22.184 |
| 1655 | LEU214 | O | 50.924 | 47.847 | 22.324 |
| 1656 | LEU215 | N | 52.984 | 48.277 | 23.122 |
| 1657 | LEU215 | CA | 52.814 | 47.389 | 24.273 |
| 1658 | LEU215 | CB | 54.181 | 47.158 | 24.908 |
| 1659 | LEU215 | CG | 54.103 | 46.281 | 26.152 |
| 1660 | LEU215 | CD1 | 53.349 | 44.985 | 25.879 |
| 1661 | LEU215 | CD2 | 55.494 | 45.992 | 26.704 |
| 1662 | LEU215 | C | 51.847 | 48.005 | 25.28 |
| 1663 | LEU215 | O | 50.996 | 47.288 | 25.819 |
| 1664 | GLY216 | N | 51.79 | 49.326 | 25.301 |
| 1665 | GLY216 | CA | 50.839 | 50.045 | 26.145 |
| 1666 | GLY216 | C | 49.421 | 49.817 | 25.654 |
| 1667 | GLY216 | O | 48.555 | 49.41 | 26.438 |
| 1668 | TRP217 | N | 49.24 | 49.9 | 24.346 |
| 1669 | TRP217 | CA | 47.928 | 49.645 | 23.754 |
| 1670 | TRP217 | CB | 47.987 | 49.982 | 22.27 |
| 1671 | TRP217 | CG | 46.688 | 49.691 | 21.55 |
| 1672 | TRP217 | CD1 | 45.524 | 50.424 | 21.625 |
| 1673 | TRP217 | NE1 | 44.588 | 49.823 | 20.849 |
| 1674 | TRP217 | CE2 | 45.081 | 48.719 | 20.257 |
| 1675 | TRP217 | CZ2 | 44.525 | 47.781 | 19.399 |
| 1676 | TRP217 | CH2 | 45.298 | 46.719 | 18.944 |
| 1677 | TRP217 | CZ3 | 46.624 | 46.591 | 19.346 |
| 1678 | TRP217 | CE3 | 47.189 | 47.524 | 20.205 |
| 1679 | TRP217 | CD2 | 46.421 | 48.586 | 20.661 |
| 1680 | TRP217 | C | 47.487 | 48.193 | 23.933 |
| 1681 | TRP217 | O | 46.36 | 47.973 | 24.392 |
| 1682 | TRP218 | N | 48.418 | 47.255 | 23.846 |
| 1683 | TRP218 | CA | 48.065 | 45.847 | 24.044 |
| 1684 | TRP218 | CB | 49.276 | 44.988 | 23.689 |
| 1685 | TRP218 | CG | 48.974 | 43.51 | 23.524 |
| 1686 | TRP218 | CD1 | 48.616 | 42.882 | 22.352 |
| 1687 | TRP218 | NE1 | 48.432 | 41.563 | 22.604 |
| 1688 | TRP218 | CE2 | 48.65 | 41.282 | 23.904 |
| 1689 | TRP218 | CZ2 | 48.585 | 40.105 | 24.633 |
| 1690 | TRP218 | CH2 | 48.857 | 40.124 | 25.995 |
| 1691 | TRP218 | CZ3 | 49.196 | 41.314 | 26.628 |
| 1692 | TRP218 | CE3 | 49.27 | 42.498 | 25.901 |
| 1693 | TRP218 | CD2 | 48.997 | 42.485 | 24.544 |
| 1694 | TRP218 | C | 47.658 | 45.589 | 25.495 |
| 1695 | TRP218 | O | 46.551 | 45.086 | 25.727 |
| 1696 | LEU219 | N | 48.369 | 46.205 | 26.426 |
| 1697 | LEU219 | CA | 48.072 | 46.021 | 27.85 |
| 1698 | LEU219 | CB | 49.247 | 46.554 | 28.665 |
| 1699 | LEU219 | CG | 50.52 | 45.735 | 28.469 |
| 1700 | LEU219 | CD1 | 51.69 | 46.373 | 29.208 |
| 1701 | LEU219 | CD2 | 50.337 | 44.289 | 28.914 |
| 1702 | LEU219 | C | 46.795 | 46.736 | 28.299 |
| 1703 | LEU219 | O | 46.024 | 46.154 | 29.074 |
| 1704 | CYS220 | N | 46.444 | 47.845 | 27.666 |
| 1705 | CYS220 | CA | 45.202 | 48.525 | 28.052 |
| 1706 | CYS220 | CB | 45.288 | 50.023 | 27.767 |
| 1707 | CYS220 | SG | 45.291 | 50.544 | 26.037 |
| 1708 | CYS220 | C | 43.982 | 47.91 | 27.364 |
| 1709 | CYS220 | O | 42.879 | 47.992 | 27.916 |
| 1710 | GLU221 | N | 44.214 | 47.072 | 26.361 |
| 1711 | GLU221 | CA | 43.119 | 46.313 | 25.745 |
| 1712 | GLU221 | CB | 43.508 | 45.882 | 24.335 |
| 1713 | GLU221 | CG | 43.683 | 47.065 | 23.393 |
| 1714 | GLU221 | CD | 42.379 | 47.84 | 23.23 |
| 1715 | GLU221 | OE1 | 41.388 | 47.21 | 22.895 |
| 1716 | GLU221 | OE2 | 42.458 | 49.061 | 23.216 |
| 1717 | GLU221 | C | 42.772 | 45.064 | 26.554 |
| 1718 | GLU221 | O | 41.756 | 44.418 | 26.274 |
| 1719 | ARG222 | N | 43.548 | 44.777 | 27.59 |
| 1720 | ARG222 | CA | 43.252 | 43.633 | 28.452 |
| 1721 | ARG222 | CB | 44.541 | 43.198 | 29.146 |
| 1722 | ARG222 | CG | 45.646 | 42.904 | 28.136 |
| 1723 | ARG222 | CD | 45.292 | 41.729 | 27.232 |
| 1724 | ARG222 | NE | 45.617 | 42.034 | 25.834 |
| 1725 | ARG222 | CZ | 44.688 | 42.163 | 24.886 |
| 1726 | ARG222 | NH1 | 43.414 | 41.876 | 25.159 |
| 1727 | ARG222 | NH2 | 45.044 | 42.487 | 23.642 |
| 1728 | ARG222 | C | 42.201 | 43.99 | 29.5 |
| 1729 | ARG222 | O | 41.588 | 43.08 | 30.076 |
| 1730 | GLN223 | N | 41.925 | 45.275 | 29.674 |
| 1731 | GLN223 | CA | 40.9 | 45.693 | 30.633 |
| 1732 | GLN223 | CB | 41.158 | 47.128 | 31.077 |
| 1733 | GLN223 | CG | 40.121 | 47.56 | 32.111 |
| 1734 | GLN223 | CD | 40.563 | 48.832 | 32.823 |
| 1735 | GLN223 | OE1 | 41.053 | 49.784 | 32.2 |
| 1736 | GLN223 | NE2 | 40.456 | 48.795 | 34.138 |
| 1737 | GLN223 | C | 39.502 | 45.573 | 30.04 |
| 1738 | GLN223 | O | 39.087 | 46.335 | 29.16 |
| 1739 | LEU224 | N | 38.775 | 44.612 | 30.574 |
| 1740 | LEU224 | CA | 37.399 | 44.356 | 30.158 |
| 1741 | LEU224 | CB | 37.096 | 42.908 | 30.517 |
| 1742 | LEU224 | CG | 37.949 | 41.984 | 29.664 |
| 1743 | LEU224 | CD1 | 37.705 | 40.538 | 30.048 |
| 1744 | LEU224 | CD2 | 37.67 | 42.202 | 28.18 |
| 1745 | LEU224 | C | 36.443 | 45.309 | 30.864 |
| 1746 | LEU224 | O | 36.812 | 45.898 | 31.887 |
| 1747 | PRO225 | N | 35.209 | 45.406 | 30.378 |
| 1748 | PRO225 | CA | 34.2 | 46.305 | 30.977 |
| 1749 | PRO225 | CB | 33.043 | 46.286 | 30.025 |
| 1750 | PRO225 | CG | 33.308 | 45.279 | 28.919 |
| 1751 | PRO225 | CD | 34.695 | 44.722 | 29.183 |
| 1752 | PRO225 | C | 33.723 | 45.934 | 32.396 |
| 1753 | PRO225 | O | 32.934 | 46.678 | 32.985 |
| 1754 | SER226 | N | 34.218 | 44.835 | 32.949 |
| 1755 | SER226 | CA | 33.978 | 44.497 | 34.356 |
| 1756 | SER226 | CB | 34.204 | 43.004 | 34.54 |
| 1757 | SER226 | OG | 35.609 | 42.78 | 34.473 |
| 1758 | SER226 | C | 34.983 | 45.195 | 35.271 |
| 1759 | SER226 | O | 34.911 | 45.044 | 36.494 |
| 1760 | GLY227 | N | 35.986 | 45.824 | 34.677 |
| 1761 | GLY227 | CA | 37.024 | 46.493 | 35.447 |
| 1762 | GLY227 | C | 38.35 | 45.743 | 35.398 |
| 1763 | GLY227 | O | 39.418 | 46.372 | 35.4 |
| 1764 | GLY228 | N | 38.281 | 44.429 | 35.25 |
| 1765 | GLY228 | CA | 39.478 | 43.6 | 35.389 |
| 1766 | GLY228 | C | 40.235 | 43.358 | 34.095 |
| 1767 | GLY228 | O | 39.674 | 43.334 | 32.994 |
| 1768 | LEU229 | N | 41.518 | 43.12 | 34.281 |
| 1769 | LEU229 | CA | 42.45 | 42.888 | 33.18 |
| 1770 | LEU229 | CB | 43.805 | 43.518 | 33.535 |
| 1771 | LEU229 | CG | 43.984 | 45.025 | 33.28 |
| 1772 | LEU229 | CD1 | 42.942 | 45.936 | 33.919 |
| 1773 | LEU229 | CD2 | 45.352 | 45.471 | 33.769 |
| 1774 | LEU229 | C | 42.632 | 41.384 | 32.987 |
| 1775 | LEU229 | O | 42.696 | 40.636 | 33.974 |
| 1776 | ASN230 | N | 42.547 | 40.939 | 31.745 |
| 1777 | ASN230 | CA | 42.849 | 39.541 | 31.428 |
| 1778 | ASN230 | CB | 41.954 | 39.013 | 30.306 |
| 1779 | ASN230 | CG | 42.032 | 39.815 | 29.01 |
| 1780 | ASN230 | OD1 | 43.113 | 40.026 | 28.45 |
| 1781 | ASN230 | ND2 | 40.866 | 40.022 | 28.429 |
| 1782 | ASN230 | C | 44.325 | 39.382 | 31.081 |
| 1783 | ASN230 | O | 45.112 | 40.33 | 31.19 |
| 1784 | GLY231 | N | 44.7 | 38.164 | 30.732 |
| 1785 | GLY231 | CA | 46.107 | 37.867 | 30.443 |
| 1786 | GLY231 | C | 46.447 | 38.093 | 28.978 |
| 1787 | GLY231 | O | 47.574 | 38.476 | 28.641 |
| 1788 | ARG232 | N | 45.506 | 37.714 | 28.133 |
| 1789 | ARG232 | CA | 45.63 | 37.864 | 26.685 |
| 1790 | ARG232 | CB | 46.574 | 36.776 | 26.164 |
| 1791 | ARG232 | CG | 46.11 | 35.38 | 26.535 |
| 1792 | ARG232 | CD | 47.139 | 34.331 | 26.134 |
| 1793 | ARG232 | NE | 46.706 | 32.995 | 26.569 |
| 1794 | ARG232 | CZ | 47.18 | 32.388 | 27.659 |
| 1795 | ARG232 | NH1 | 48.123 | 32.976 | 28.399 |
| 1796 | ARG232 | NH2 | 46.724 | 31.181 | 27.999 |
| 1797 | ARG232 | C | 44.219 | 37.785 | 26.093 |
| 1798 | ARG232 | O | 43.295 | 37.358 | 26.802 |
| 1799 | PRO233 | N | 44.05 | 38.191 | 24.84 |
| 1800 | PRO233 | CA | 42.715 | 38.497 | 24.304 |
| 1801 | PRO233 | CB | 42.932 | 38.832 | 22.86 |
| 1802 | PRO233 | CG | 44.423 | 38.917 | 22.585 |
| 1803 | PRO233 | CD | 45.105 | 38.606 | 23.905 |
| 1804 | PRO233 | C | 41.69 | 37.376 | 24.456 |
| 1805 | PRO233 | O | 42.009 | 36.184 | 24.392 |
| 1806 | GLU234 | N | 40.484 | 37.817 | 24.789 |
| 1807 | GLU234 | CA | 39.278 | 36.977 | 24.919 |
| 1808 | GLU234 | CB | 39.106 | 36.09 | 23.687 |
| 1809 | GLU234 | CG | 38.852 | 36.909 | 22.426 |
| 1810 | GLU234 | CD | 38.719 | 35.987 | 21.217 |
| 1811 | GLU234 | OE1 | 39.514 | 35.064 | 21.113 |
| 1812 | GLU234 | OE2 | 37.901 | 36.298 | 20.363 |
| 1813 | GLU234 | C | 39.259 | 36.107 | 26.175 |
| 1814 | GLU234 | O | 38.518 | 35.118 | 26.213 |
| 1815 | LYS235 | N | 39.999 | 36.501 | 27.197 |
| 1816 | LYS235 | CA | 39.978 | 35.76 | 28.459 |
| 1817 | LYS235 | CB | 41.407 | 35.456 | 28.878 |
| 1818 | LYS235 | CG | 42.028 | 34.359 | 28.028 |
| 1819 | LYS235 | CD | 43.49 | 34.184 | 28.404 |
| 1820 | LYS235 | CE | 43.668 | 34.018 | 29.907 |
| 1821 | LYS235 | NZ | 45.096 | 33.997 | 30.255 |
| 1822 | LYS235 | C | 39.299 | 36.532 | 29.579 |
| 1823 | LYS235 | O | 39.113 | 37.754 | 29.504 |
| 1824 | LEU236 | N | 38.936 | 35.779 | 30.602 |
| 1825 | LEU236 | CA | 38.415 | 36.328 | 31.857 |
| 1826 | LEU236 | CB | 38.096 | 35.155 | 32.777 |
| 1827 | LEU236 | CG | 36.878 | 34.399 | 32.275 |
| 1828 | LEU236 | CD1 | 36.694 | 33.077 | 33.007 |
| 1829 | LEU236 | CD2 | 35.642 | 35.273 | 32.399 |
| 1830 | LEU236 | C | 39.419 | 37.245 | 32.543 |
| 1831 | LEU236 | O | 40.636 | 37.032 | 32.474 |
| 1832 | PRO237 | N | 38.892 | 38.299 | 33.143 |
| 1833 | PRO237 | CA | 39.686 | 39.157 | 34.015 |
| 1834 | PRO237 | CB | 38.79 | 40.307 | 34.339 |
| 1835 | PRO237 | CG | 37.388 | 40.016 | 33.837 |
| 1836 | PRO237 | CD | 37.479 | 38.673 | 33.14 |
| 1837 | PRO237 | C | 40.077 | 38.404 | 35.277 |
| 1838 | PRO237 | O | 39.273 | 37.652 | 35.835 |
| 1839 | ASP238 | N | 41.308 | 38.598 | 35.708 |
| 1840 | ASP238 | CA | 41.799 | 37.909 | 36.908 |
| 1841 | ASP238 | CB | 42.324 | 36.544 | 36.465 |
| 1842 | ASP238 | CG | 42.739 | 35.677 | 37.649 |
| 1843 | ASP238 | OD1 | 43.81 | 35.932 | 38.187 |
| 1844 | ASP238 | OD2 | 42.022 | 34.735 | 37.955 |
| 1845 | ASP238 | C | 42.903 | 38.747 | 37.545 |
| 1846 | ASP238 | O | 43.809 | 39.188 | 36.83 |
| 1847 | VAL239 | N | 42.946 | 38.816 | 38.868 |
| 1848 | VAL239 | CA | 43.89 | 39.735 | 39.531 |
| 1849 | VAL239 | CB | 43.523 | 39.876 | 41.003 |
| 1850 | VAL239 | CG1 | 42.254 | 40.697 | 41.176 |
| 1851 | VAL239 | CG2 | 43.398 | 38.525 | 41.69 |
| 1852 | VAL239 | C | 45.386 | 39.401 | 39.409 |
| 1853 | VAL239 | O | 46.179 | 40.347 | 39.48 |
| 1854 | CYS240 | N | 45.776 | 38.204 | 38.991 |
| 1855 | CYS240 | CA | 47.214 | 37.99 | 38.77 |
| 1856 | CYS240 | CB | 47.572 | 36.506 | 38.842 |
| 1857 | CYS240 | SG | 46.878 | 35.394 | 37.595 |
| 1858 | CYS240 | C | 47.644 | 38.581 | 37.428 |
| 1859 | CYS240 | O | 48.65 | 39.299 | 37.381 |
| 1860 | TYR241 | N | 46.71 | 38.609 | 36.49 |
| 1861 | TYR241 | CA | 46.979 | 39.173 | 35.169 |
| 1862 | TYR241 | CB | 46.015 | 38.553 | 34.168 |
| 1863 | TYR241 | CG | 46.153 | 37.044 | 33.993 |
| 1864 | TYR241 | CD1 | 45.052 | 36.221 | 34.196 |
| 1865 | TYR241 | CE1 | 45.174 | 34.847 | 34.037 |
| 1866 | TYR241 | CZ | 46.397 | 34.302 | 33.668 |
| 1867 | TYR241 | OH | 46.523 | 32.937 | 33.523 |
| 1868 | TYR241 | CE2 | 47.495 | 35.123 | 33.452 |
| 1869 | TYR241 | CD2 | 47.371 | 36.496 | 33.613 |
| 1870 | TYR241 | C | 46.755 | 40.673 | 35.212 |
| 1871 | TYR241 | O | 47.52 | 41.434 | 34.607 |
| 1872 | SER242 | N | 45.921 | 41.076 | 36.155 |
| 1873 | SER242 | CA | 45.677 | 42.492 | 36.405 |
| 1874 | SER242 | CB | 44.526 | 42.639 | 37.393 |
| 1875 | SER242 | OG | 43.373 | 42.029 | 36.826 |
| 1876 | SER242 | C | 46.927 | 43.147 | 36.971 |
| 1877 | SER242 | O | 47.392 | 44.127 | 36.379 |
| 1878 | TRP243 | N | 47.607 | 42.472 | 37.885 |
| 1879 | TRP243 | CA | 48.855 | 43.027 | 38.414 |
| 1880 | TRP243 | CB | 49.265 | 42.296 | 39.687 |
| 1881 | TRP243 | CG | 50.708 | 42.579 | 40.072 |
| 1882 | TRP243 | CD1 | 51.751 | 41.68 | 40.048 |
| 1883 | TRP243 | NE1 | 52.884 | 42.322 | 40.424 |
| 1884 | TRP243 | CE2 | 52.641 | 43.616 | 40.699 |
| 1885 | TRP243 | CZ2 | 53.461 | 44.664 | 41.091 |
| 1886 | TRP243 | CH2 | 52.917 | 45.926 | 41.293 |
| 1887 | TRP243 | CZ3 | 51.557 | 46.143 | 41.103 |
| 1888 | TRP243 | CE3 | 50.727 | 45.1 | 40.71 |
| 1889 | TRP243 | CD2 | 51.265 | 43.839 | 40.506 |
| 1890 | TRP243 | C | 50.004 | 42.943 | 37.416 |
| 1891 | TRP243 | O | 50.725 | 43.936 | 37.265 |
| 1892 | TRP244 | N | 50.044 | 41.908 | 36.594 |
| 1893 | TRP244 | CA | 51.157 | 41.792 | 35.645 |
| 1894 | TRP244 | CB | 51.203 | 40.369 | 35.102 |
| 1895 | TRP244 | CG | 51.608 | 39.348 | 36.148 |
| 1896 | TRP244 | CD1 | 52.419 | 39.57 | 37.238 |
| 1897 | TRP244 | NE1 | 52.536 | 38.413 | 37.934 |
| 1898 | TRP244 | CE2 | 51.837 | 37.422 | 37.35 |
| 1899 | TRP244 | CZ2 | 51.646 | 36.088 | 37.676 |
| 1900 | TRP244 | CH2 | 50.853 | 35.286 | 36.863 |
| 1901 | TRP244 | CZ3 | 50.252 | 35.817 | 35.727 |
| 1902 | TRP244 | CE3 | 50.436 | 37.153 | 35.394 |
| 1903 | TRP244 | CD2 | 51.227 | 37.955 | 36.201 |
| 1904 | TRP244 | C | 51.053 | 42.804 | 34.507 |
| 1905 | TRP244 | O | 52.042 | 43.492 | 34.221 |
| 1906 | VAL245 | N | 49.834 | 43.103 | 34.089 |
| 1907 | VAL245 | CA | 49.639 | 44.115 | 33.051 |
| 1908 | VAL245 | CB | 48.261 | 43.918 | 32.429 |
| 1909 | VAL245 | CG1 | 47.894 | 45.083 | 31.523 |
| 1910 | VAL245 | CG2 | 48.162 | 42.604 | 31.669 |
| 1911 | VAL245 | C | 49.742 | 45.526 | 33.622 |
| 1912 | VAL245 | O | 50.425 | 46.364 | 33.022 |
| 1913 | LEU246 | N | 49.348 | 45.688 | 34.876 |
| 1914 | LEU246 | CA | 49.403 | 46.996 | 35.537 |
| 1915 | LEU246 | CB | 48.655 | 46.866 | 36.86 |
| 1916 | LEU246 | CG | 48.499 | 48.193 | 37.587 |
| 1917 | LEU246 | CD1 | 47.577 | 49.118 | 36.803 |
| 1918 | LEU246 | CD2 | 47.946 | 47.966 | 38.989 |
| 1919 | LEU246 | C | 50.841 | 47.405 | 35.833 |
| 1920 | LEU246 | O | 51.265 | 48.507 | 35.46 |
| 1921 | ALA247 | N | 51.635 | 46.438 | 36.256 |
| 1922 | ALA247 | CA | 53.027 | 46.712 | 36.587 |
| 1923 | ALA247 | CB | 53.55 | 45.571 | 37.442 |
| 1924 | ALA247 | C | 53.889 | 46.871 | 35.344 |
| 1925 | ALA247 | O | 54.648 | 47.843 | 35.288 |
| 1926 | SER248 | N | 53.549 | 46.174 | 34.27 |
| 1927 | SER248 | CA | 54.3 | 46.342 | 33.019 |
| 1928 | SER248 | CB | 54.031 | 45.151 | 32.112 |
| 1929 | SER248 | OG | 54.539 | 43.996 | 32.76 |
| 1930 | SER248 | C | 53.908 | 47.624 | 32.29 |
| 1931 | SER248 | O | 54.78 | 48.288 | 31.717 |
| 1932 | LEU249 | N | 52.706 | 48.101 | 32.565 |
| 1933 | LEU249 | CA | 52.229 | 49.354 | 31.989 |
| 1934 | LEU249 | CB | 50.715 | 49.36 | 32.161 |
| 1935 | LEU249 | CG | 50.019 | 50.297 | 31.19 |
| 1936 | LEU249 | CD1 | 50.396 | 49.964 | 29.754 |
| 1937 | LEU249 | CD2 | 48.508 | 50.232 | 31.369 |
| 1938 | LEU249 | C | 52.865 | 50.536 | 32.719 |
| 1939 | LEU249 | O | 53.298 | 51.5 | 32.073 |
| 1940 | LYS250 | N | 53.184 | 50.324 | 33.986 |
| 1941 | LYS250 | CA | 53.925 | 51.322 | 34.759 |
| 1942 | LYS250 | CB | 53.729 | 51.002 | 36.237 |
| 1943 | LYS250 | CG | 54.551 | 51.917 | 37.138 |
| 1944 | LYS250 | CD | 54.169 | 53.383 | 36.975 |
| 1945 | LYS250 | CE | 55.018 | 54.266 | 37.88 |
| 1946 | LYS250 | NZ | 54.875 | 53.855 | 39.285 |
| 1947 | LYS250 | C | 55.416 | 51.308 | 34.419 |
| 1948 | LYS250 | O | 55.999 | 52.383 | 34.231 |
| 1949 | ILE251 | N | 55.937 | 50.144 | 34.059 |
| 1950 | ILE251 | CA | 57.351 | 50.027 | 33.678 |
| 1951 | ILE251 | CB | 57.722 | 48.542 | 33.671 |
| 1952 | ILE251 | CG2 | 59.112 | 48.315 | 33.084 |
| 1953 | ILE251 | CG1 | 57.649 | 47.952 | 35.073 |
| 1954 | ILE251 | CD1 | 57.92 | 46.453 | 35.062 |
| 1955 | ILE251 | C | 57.637 | 50.636 | 32.305 |
| 1956 | ILE251 | O | 58.722 | 51.194 | 32.092 |
| 1957 | ILE252 | N | 56.634 | 50.665 | 31.442 |
| 1958 | ILE252 | CA | 56.806 | 51.297 | 30.134 |
| 1959 | ILE252 | CB | 56.091 | 50.466 | 29.076 |
| 1960 | ILE252 | CG2 | 56.634 | 49.043 | 29.069 |
| 1961 | ILE252 | CG1 | 54.587 | 50.449 | 29.292 |
| 1962 | ILE252 | CD1 | 53.904 | 49.556 | 28.268 |
| 1963 | ILE252 | C | 56.322 | 52.75 | 30.099 |
| 1964 | ILE252 | O | 56.389 | 53.383 | 29.039 |
| 1965 | GLY253 | N | 55.821 | 53.259 | 31.217 |
| 1966 | GLY253 | CA | 55.406 | 54.668 | 31.309 |
| 1967 | GLY253 | C | 53.93 | 54.91 | 30.992 |
| 1968 | GLY253 | O | 53.345 | 55.905 | 31.441 |
| 1969 | ARG254 | N | 53.295 | 53.919 | 30.389 |
| 1970 | ARG254 | CA | 51.939 | 54.061 | 29.845 |
| 1971 | ARG254 | CB | 51.82 | 53.177 | 28.614 |
| 1972 | ARG254 | CG | 52.849 | 53.56 | 27.564 |
| 1973 | ARG254 | CD | 52.656 | 54.994 | 27.085 |
| 1974 | ARG254 | NE | 53.692 | 55.354 | 26.108 |
| 1975 | ARG254 | CZ | 54.796 | 56.032 | 26.431 |
| 1976 | ARG254 | NH1 | 54.983 | 56.449 | 27.686 |
| 1977 | ARG254 | NH2 | 55.701 | 56.314 | 25.493 |
| 1978 | ARG254 | C | 50.841 | 53.683 | 30.83 |
| 1979 | ARG254 | O | 49.722 | 53.365 | 30.405 |
| 1980 | LEU255 | N | 51.079 | 53.903 | 32.115 |
| 1981 | LEU255 | CA | 50.122 | 53.489 | 33.152 |
| 1982 | LEU255 | CB | 50.802 | 53.587 | 34.513 |
| 1983 | LEU255 | CG | 49.936 | 52.955 | 35.595 |
| 1984 | LEU255 | CD1 | 49.734 | 51.475 | 35.306 |
| 1985 | LEU255 | CD2 | 50.531 | 53.15 | 36.985 |
| 1986 | LEU255 | C | 48.867 | 54.366 | 33.154 |
| 1987 | LEU255 | O | 47.778 | 53.872 | 33.466 |
| 1988 | HIS256 | N | 48.975 | 55.508 | 32.495 |
| 1989 | HIS256 | CA | 47.874 | 56.46 | 32.333 |
| 1990 | HIS256 | CB | 48.494 | 57.805 | 31.953 |
| 1991 | HIS256 | CG | 49.486 | 57.746 | 30.8 |
| 1992 | HIS256 | ND1 | 50.829 | 57.847 | 30.882 |
| 1993 | HIS256 | CE1 | 51.362 | 57.745 | 29.648 |
| 1994 | HIS256 | NE2 | 50.343 | 57.592 | 28.773 |
| 1995 | HIS256 | CD2 | 49.183 | 57.603 | 29.466 |
| 1996 | HIS256 | C | 46.842 | 56.041 | 31.275 |
| 1997 | HIS256 | O | 45.869 | 56.77 | 31.054 |
| 1998 | TRP257 | N | 47.066 | 54.919 | 30.604 |
| 1999 | TRP257 | CA | 46.067 | 54.392 | 29.674 |
| 2000 | TRP257 | CB | 46.761 | 53.764 | 28.469 |
| 2001 | TRP257 | CG | 47.306 | 54.766 | 27.47 |
| 2002 | TRP257 | CD1 | 46.924 | 56.082 | 27.333 |
| 2003 | TRP257 | NE1 | 47.642 | 56.637 | 26.324 |
| 2004 | TRP257 | CE2 | 48.48 | 55.74 | 25.772 |
| 2005 | TRP257 | CZ2 | 49.39 | 55.819 | 24.728 |
| 2006 | TRP257 | CH2 | 50.126 | 54.697 | 24.367 |
| 2007 | TRP257 | CZ3 | 49.955 | 53.496 | 25.049 |
| 2008 | TRP257 | CE3 | 49.048 | 53.408 | 26.099 |
| 2009 | TRP257 | CD2 | 48.312 | 54.525 | 26.462 |
| 2010 | TRP257 | C | 45.151 | 53.359 | 30.331 |
| 2011 | TRP257 | O | 44.166 | 52.939 | 29.711 |
| 2012 | ILE258 | N | 45.453 | 52.953 | 31.554 |
| 2013 | ILE258 | CA | 44.57 | 51.994 | 32.227 |
| 2014 | ILE258 | CB | 45.409 | 51.032 | 33.077 |
| 2015 | ILE258 | CG2 | 45.788 | 51.633 | 34.426 |
| 2016 | ILE258 | CG1 | 44.685 | 49.707 | 33.293 |
| 2017 | ILE258 | CD1 | 44.452 | 48.992 | 31.966 |
| 2018 | ILE258 | C | 43.54 | 52.751 | 33.07 |
| 2019 | ILE258 | O | 43.832 | 53.818 | 33.626 |
| 2020 | ASP259 | N | 42.31 | 52.269 | 33.073 |
| 2021 | ASP259 | CA | 41.296 | 52.89 | 33.924 |
| 2022 | ASP259 | CB | 39.902 | 52.613 | 33.369 |
| 2023 | ASP259 | CG | 38.872 | 53.465 | 34.104 |
| 2024 | ASP259 | OD1 | 38.67 | 53.204 | 35.286 |
| 2025 | ASP259 | OD2 | 38.451 | 54.462 | 33.54 |
| 2026 | ASP259 | C | 41.443 | 52.338 | 35.338 |
| 2027 | ASP259 | O | 40.881 | 51.287 | 35.69 |
| 2028 | ARG260 | N | 42.021 | 53.168 | 36.19 |
| 2029 | ARG260 | CA | 42.351 | 52.748 | 37.552 |
| 2030 | ARG260 | CB | 43.276 | 53.793 | 38.159 |
| 2031 | ARG260 | CG | 44.569 | 53.928 | 37.366 |
| 2032 | ARG260 | CD | 45.47 | 54.977 | 38.003 |
| 2033 | ARG260 | NE | 46.75 | 55.115 | 37.29 |
| 2034 | ARG260 | CZ | 47.088 | 56.219 | 36.619 |
| 2035 | ARG260 | NH1 | 46.202 | 57.205 | 36.465 |
| 2036 | ARG260 | NH2 | 48.289 | 56.31 | 36.047 |
| 2037 | ARG260 | C | 41.142 | 52.58 | 38.465 |
| 2038 | ARG260 | O | 41.143 | 51.627 | 39.247 |
| 2039 | GLU261 | N | 40.031 | 53.234 | 38.159 |
| 2040 | GLU261 | CA | 38.859 | 53.123 | 39.035 |
| 2041 | GLU261 | CB | 37.947 | 54.322 | 38.809 |
| 2042 | GLU261 | CG | 38.639 | 55.636 | 39.157 |
| 2043 | GLU261 | CD | 39.093 | 55.636 | 40.615 |
| 2044 | GLU261 | OE1 | 38.24 | 55.783 | 41.478 |
| 2045 | GLU261 | OE2 | 40.292 | 55.525 | 40.828 |
| 2046 | GLU261 | C | 38.077 | 51.84 | 38.776 |
| 2047 | GLU261 | O | 37.568 | 51.229 | 39.725 |
| 2048 | LYS262 | N | 38.165 | 51.33 | 37.56 |
| 2049 | LYS262 | CA | 37.495 | 50.07 | 37.25 |
| 2050 | LYS262 | CB | 37.232 | 49.999 | 35.753 |
| 2051 | LYS262 | CG | 36.219 | 51.042 | 35.306 |
| 2052 | LYS262 | CD | 35.952 | 50.925 | 33.811 |
| 2053 | LYS262 | CE | 34.942 | 51.964 | 33.342 |
| 2054 | LYS262 | NZ | 34.685 | 51.829 | 31.899 |
| 2055 | LYS262 | C | 38.345 | 48.882 | 37.675 |
| 2056 | LYS262 | O | 37.803 | 47.93 | 38.248 |
| 2057 | LEU263 | N | 39.657 | 49.056 | 37.657 |
| 2058 | LEU263 | CA | 40.531 | 47.962 | 38.078 |
| 2059 | LEU263 | CB | 41.932 | 48.188 | 37.524 |
| 2060 | LEU263 | CG | 42.86 | 47.037 | 37.897 |
| 2061 | LEU263 | CD1 | 42.278 | 45.694 | 37.466 |
| 2062 | LEU263 | CD2 | 44.25 | 47.24 | 37.308 |
| 2063 | LEU263 | C | 40.569 | 47.888 | 39.597 |
| 2064 | LEU263 | O | 40.441 | 46.793 | 40.158 |
| 2065 | ARG264 | N | 40.405 | 49.04 | 40.224 |
| 2066 | ARG264 | CA | 40.317 | 49.093 | 41.677 |
| 2067 | ARG264 | CB | 40.356 | 50.545 | 42.134 |
| 2068 | ARG264 | CG | 40.062 | 50.619 | 43.623 |
| 2069 | ARG264 | CD | 40.165 | 52.03 | 44.185 |
| 2070 | ARG264 | NE | 39.734 | 52.023 | 45.593 |
| 2071 | ARG264 | CZ | 40.534 | 51.731 | 46.622 |
| 2072 | ARG264 | NH1 | 41.846 | 51.572 | 46.43 |
| 2073 | ARG264 | NH2 | 40.033 | 51.697 | 47.859 |
| 2074 | ARG264 | C | 39.038 | 48.438 | 42.173 |
| 2075 | ARG264 | O | 39.139 | 47.511 | 42.982 |
| 2076 | ASN265 | N | 37.93 | 48.661 | 41.482 |
| 2077 | ASN265 | CA | 36.668 | 48.048 | 41.908 |
| 2078 | ASN265 | CB | 35.492 | 48.829 | 41.333 |
| 2079 | ASN265 | CG | 35.129 | 49.997 | 42.251 |
| 2080 | ASN265 | OD1 | 34.476 | 49.808 | 43.284 |
| 2081 | ASN265 | ND2 | 35.572 | 51.187 | 41.881 |
| 2082 | ASN265 | C | 36.563 | 46.569 | 41.541 |
| 2083 | ASN265 | O | 35.943 | 45.818 | 42.304 |
| 2084 | PHE266 | N | 37.365 | 46.109 | 40.595 |
| 2085 | PHE266 | CA | 37.423 | 44.671 | 40.328 |
| 2086 | PHE266 | CB | 38.068 | 44.45 | 38.968 |
| 2087 | PHE266 | CG | 38.268 | 42.98 | 38.61 |
| 2088 | PHE266 | CD1 | 37.201 | 42.234 | 38.127 |
| 2089 | PHE266 | CE1 | 37.381 | 40.897 | 37.8 |
| 2090 | PHE266 | CZ | 38.628 | 40.306 | 37.956 |
| 2091 | PHE266 | CE2 | 39.697 | 41.052 | 38.434 |
| 2092 | PHE266 | CD2 | 39.517 | 42.39 | 38.76 |
| 2093 | PHE266 | C | 38.242 | 43.951 | 41.396 |
| 2094 | PHE266 | O | 37.817 | 42.898 | 41.886 |
| 2095 | ILE267 | N | 39.251 | 44.628 | 41.918 |
| 2096 | ILE267 | CA | 40.071 | 44.044 | 42.98 |
| 2097 | ILE267 | CB | 41.414 | 44.766 | 42.98 |
| 2098 | ILE267 | CG2 | 42.315 | 44.234 | 44.081 |
| 2099 | ILE267 | CG1 | 42.111 | 44.612 | 41.634 |
| 2100 | ILE267 | CD1 | 43.459 | 45.326 | 41.627 |
| 2101 | ILE267 | C | 39.382 | 44.166 | 44.343 |
| 2102 | ILE267 | O | 39.485 | 43.249 | 45.169 |
| 2103 | LEU268 | N | 38.482 | 45.13 | 44.461 |
| 2104 | LEU268 | CA | 37.645 | 45.245 | 45.66 |
| 2105 | LEU268 | CB | 36.966 | 46.612 | 45.659 |
| 2106 | LEU268 | CG | 37.939 | 47.78 | 45.775 |
| 2107 | LEU268 | CD1 | 37.249 | 49.097 | 45.444 |
| 2108 | LEU268 | CD2 | 38.594 | 47.851 | 47.148 |
| 2109 | LEU268 | C | 36.57 | 44.159 | 45.664 |
| 2110 | LEU268 | O | 36.359 | 43.513 | 46.698 |
| 2111 | ALA269 | N | 36.14 | 43.775 | 44.471 |
| 2112 | ALA269 | CA | 35.165 | 42.694 | 44.301 |
| 2113 | ALA269 | CB | 34.566 | 42.827 | 42.904 |
| 2114 | ALA269 | C | 35.763 | 41.294 | 44.457 |
| 2115 | ALA269 | O | 35.019 | 40.313 | 44.579 |
| 2116 | CYS270 | N | 37.082 | 41.206 | 44.522 |
| 2117 | CYS270 | CA | 37.739 | 39.927 | 44.785 |
| 2118 | CYS270 | CB | 39.056 | 39.868 | 44.02 |
| 2119 | CYS270 | SG | 38.912 | 39.869 | 42.22 |
| 2120 | CYS270 | C | 38.009 | 39.71 | 46.273 |
| 2121 | CYS270 | O | 38.47 | 38.622 | 46.642 |
| 2122 | GLN271 | N | 37.717 | 40.696 | 47.108 |
| 2123 | GLN271 | CA | 37.932 | 40.538 | 48.552 |
| 2124 | GLN271 | CB | 37.871 | 41.908 | 49.217 |
| 2125 | GLN271 | CG | 38.867 | 42.853 | 48.569 |
| 2126 | GLN271 | CD | 38.911 | 44.207 | 49.266 |
| 2127 | GLN271 | OE1 | 38.288 | 44.432 | 50.309 |
| 2128 | GLN271 | NE2 | 39.785 | 45.05 | 48.748 |
| 2129 | GLN271 | C | 36.856 | 39.657 | 49.168 |
| 2130 | GLN271 | O | 35.669 | 39.793 | 48.851 |
| 2131 | ASP272 | N | 37.265 | 38.715 | 49.995 |
| 2132 | ASP272 | CA | 36.256 | 37.952 | 50.724 |
| 2133 | ASP272 | CB | 36.746 | 36.522 | 50.939 |
| 2134 | ASP272 | CG | 35.623 | 35.521 | 51.191 |
| 2135 | ASP272 | OD1 | 34.541 | 35.956 | 51.577 |
| 2136 | ASP272 | OD2 | 35.795 | 34.375 | 50.796 |
| 2137 | ASP272 | C | 36.002 | 38.671 | 52.044 |
| 2138 | ASP272 | O | 36.919 | 39.144 | 52.725 |
| 2139 | GLU273 | N | 34.728 | 38.834 | 52.355 |
| 2140 | GLU273 | CA | 34.356 | 39.462 | 53.622 |
| 2141 | GLU273 | CB | 32.971 | 40.076 | 53.462 |
| 2142 | GLU273 | CG | 33.01 | 41.15 | 52.379 |
| 2143 | GLU273 | CD | 31.612 | 41.669 | 52.066 |
| 2144 | GLU273 | OE1 | 30.702 | 40.852 | 52.075 |
| 2145 | GLU273 | OE2 | 31.509 | 42.82 | 51.666 |
| 2146 | GLU273 | C | 34.394 | 38.415 | 54.729 |
| 2147 | GLU273 | O | 34.71 | 38.717 | 55.885 |
| 2148 | GLU274 | N | 34.236 | 37.167 | 54.323 |
| 2149 | GLU274 | CA | 34.507 | 36.046 | 55.219 |
| 2150 | GLU274 | CB | 33.689 | 34.837 | 54.776 |
| 2151 | GLU274 | CG | 32.196 | 35.146 | 54.719 |
| 2152 | GLU274 | CD | 31.661 | 35.505 | 56.103 |
| 2153 | GLU274 | OE1 | 32.124 | 34.911 | 57.067 |
| 2154 | GLU274 | OE2 | 30.793 | 36.364 | 56.165 |
| 2155 | GLU274 | C | 35.992 | 35.729 | 55.111 |
| 2156 | GLU274 | O | 36.461 | 35.35 | 54.032 |
| 2157 | THR275 | N | 36.687 | 35.867 | 56.231 |
| 2158 | THR275 | CA | 38.156 | 35.712 | 56.348 |
| 2159 | THR275 | CB | 38.529 | 34.268 | 56.721 |
| 2160 | THR275 | OG1 | 39.945 | 34.189 | 56.84 |
| 2161 | THR275 | CG2 | 38.069 | 33.195 | 55.735 |
| 2162 | THR275 | C | 38.951 | 36.227 | 55.137 |
| 2163 | THR275 | O | 39.538 | 35.458 | 54.366 |
| 2164 | GLY276 | N | 38.914 | 37.546 | 54.999 |
| 2165 | GLY276 | CA | 39.692 | 38.336 | 54.025 |
| 2166 | GLY276 | C | 40.156 | 37.662 | 52.742 |
| 2167 | GLY276 | O | 39.373 | 37.067 | 51.998 |
| 2168 | GLY277 | N | 41.436 | 37.839 | 52.464 |
| 2169 | GLY277 | CA | 42.047 | 37.342 | 51.224 |
| 2170 | GLY277 | C | 41.424 | 37.88 | 49.931 |
| 2171 | GLY277 | O | 40.34 | 38.48 | 49.91 |
| 2172 | PHE278 | N | 42.177 | 37.697 | 48.861 |
| 2173 | PHE278 | CA | 41.684 | 37.991 | 47.511 |
| 2174 | PHE278 | CB | 42.632 | 38.924 | 46.773 |
| 2175 | PHE278 | CG | 42.68 | 40.37 | 47.25 |
| 2176 | PHE278 | CD1 | 43.555 | 40.752 | 48.257 |
| 2177 | PHE278 | CE1 | 43.605 | 42.074 | 48.671 |
| 2178 | PHE278 | CZ | 42.784 | 43.019 | 48.074 |
| 2179 | PHE278 | CE2 | 41.911 | 42.638 | 47.066 |
| 2180 | PHE278 | CD2 | 41.858 | 41.314 | 46.652 |
| 2181 | PHE278 | C | 41.549 | 36.711 | 46.696 |
| 2182 | PHE278 | O | 42.362 | 35.781 | 46.812 |
| 2183 | ALA279 | N | 40.448 | 36.649 | 45.972 |
| 2184 | ALA279 | CA | 40.16 | 35.56 | 45.04 |
| 2185 | ALA279 | CB | 38.649 | 35.45 | 44.917 |
| 2186 | ALA279 | C | 40.757 | 35.863 | 43.672 |
| 2187 | ALA279 | O | 41.172 | 36.997 | 43.409 |
| 2188 | ASP280 | N | 40.806 | 34.858 | 42.814 |
| 2189 | ASP280 | CA | 41.357 | 35.064 | 41.465 |
| 2190 | ASP280 | CB | 41.688 | 33.711 | 40.811 |
| 2191 | ASP280 | CG | 40.527 | 32.72 | 40.702 |
| 2192 | ASP280 | OD1 | 40.202 | 32.1 | 41.707 |
| 2193 | ASP280 | OD2 | 39.935 | 32.652 | 39.636 |
| 2194 | ASP280 | C | 40.414 | 35.918 | 40.612 |
| 2195 | ASP280 | O | 40.849 | 36.882 | 39.961 |
| 2196 | ARG281 | N | 39.133 | 35.599 | 40.695 |
| 2197 | ARG281 | CA | 38.051 | 36.411 | 40.148 |
| 2198 | ARG281 | CB | 37.463 | 35.679 | 38.942 |
| 2199 | ARG281 | CG | 38.52 | 35.426 | 37.875 |
| 2200 | ARG281 | CD | 37.938 | 34.729 | 36.654 |
| 2201 | ARG281 | NE | 37.351 | 33.434 | 37.02 |
| 2202 | ARG281 | CZ | 37.887 | 32.27 | 36.655 |
| 2203 | ARG281 | NH1 | 39.006 | 32.249 | 35.925 |
| 2204 | ARG281 | NH2 | 37.305 | 31.124 | 37.016 |
| 2205 | ARG281 | C | 37.013 | 36.544 | 41.261 |
| 2206 | ARG281 | O | 37.01 | 35.706 | 42.175 |
| 2207 | PRO282 | N | 36.16 | 37.557 | 41.196 |
| 2208 | PRO282 | CA | 35.226 | 37.842 | 42.294 |
| 2209 | PRO282 | CB | 34.381 | 38.98 | 41.808 |
| 2210 | PRO282 | CG | 34.908 | 39.464 | 40.468 |
| 2211 | PRO282 | CD | 36.076 | 38.556 | 40.128 |
| 2212 | PRO282 | C | 34.373 | 36.63 | 42.651 |
| 2213 | PRO282 | O | 34.098 | 35.785 | 41.793 |
| 2214 | GLY283 | N | 34.206 | 36.415 | 43.945 |
| 2215 | GLY283 | CA | 33.38 | 35.295 | 44.423 |
| 2216 | GLY283 | C | 34.154 | 34 | 44.702 |
| 2217 | GLY283 | O | 33.731 | 33.201 | 45.546 |
| 2218 | ASP284 | N | 35.241 | 33.777 | 43.976 |
| 2219 | ASP284 | CA | 36.042 | 32.554 | 44.128 |
| 2220 | ASP284 | CB | 37.148 | 32.535 | 43.081 |
| 2221 | ASP284 | CG | 36.594 | 32.578 | 41.663 |
| 2222 | ASP284 | OD1 | 35.6 | 31.912 | 41.415 |
| 2223 | ASP284 | OD2 | 37.296 | 33.125 | 40.824 |
| 2224 | ASP284 | C | 36.71 | 32.456 | 45.493 |
| 2225 | ASP284 | O | 36.668 | 33.383 | 46.312 |
| 2226 | MET285 | N | 37.282 | 31.289 | 45.735 |
| 2227 | MET285 | CA | 38.07 | 31.065 | 46.947 |
| 2228 | MET285 | CB | 38.543 | 29.617 | 46.972 |
| 2229 | MET285 | CG | 37.371 | 28.643 | 46.987 |
| 2230 | MET285 | SD | 37.825 | 26.894 | 47.02 |
| 2231 | MET285 | CE | 38.777 | 26.876 | 48.557 |
| 2232 | MET285 | C | 39.279 | 31.991 | 46.965 |
| 2233 | MET285 | O | 39.856 | 32.312 | 45.919 |
| 2234 | VAL286 | N | 39.565 | 32.506 | 48.146 |
| 2235 | VAL286 | CA | 40.704 | 33.405 | 48.327 |
| 2236 | VAL286 | CB | 40.371 | 34.354 | 49.467 |
| 2237 | VAL286 | CG1 | 39.192 | 35.22 | 49.058 |
| 2238 | VAL286 | CG2 | 40.054 | 33.601 | 50.754 |
| 2239 | VAL286 | C | 41.993 | 32.639 | 48.597 |
| 2240 | VAL286 | O | 41.972 | 31.514 | 49.111 |
| 2241 | ASP287 | N | 43.099 | 33.213 | 48.155 |
| 2242 | ASP287 | CA | 44.407 | 32.565 | 48.34 |
| 2243 | ASP287 | CB | 44.591 | 31.524 | 47.23 |
| 2244 | ASP287 | CG | 44.683 | 32.182 | 45.858 |
| 2245 | ASP287 | OD1 | 43.695 | 32.19 | 45.14 |
| 2246 | ASP287 | OD2 | 45.759 | 32.684 | 45.55 |
| 2247 | ASP287 | C | 45.538 | 33.598 | 48.324 |
| 2248 | ASP287 | O | 45.434 | 34.605 | 47.616 |
| 2249 | PRO288 | N | 46.658 | 33.292 | 48.968 |
| 2250 | PRO288 | CA | 47.727 | 34.292 | 49.169 |
| 2251 | PRO288 | CB | 48.708 | 33.634 | 50.091 |
| 2252 | PRO288 | CG | 48.253 | 32.217 | 50.394 |
| 2253 | PRO288 | CD | 46.928 | 32.036 | 49.676 |
| 2254 | PRO288 | C | 48.45 | 34.773 | 47.899 |
| 2255 | PRO288 | O | 48.891 | 35.927 | 47.875 |
| 2256 | PHE289 | N | 48.353 | 34.028 | 46.808 |
| 2257 | PHE289 | CA | 48.962 | 34.433 | 45.535 |
| 2258 | PHE289 | CB | 48.838 | 33.229 | 44.603 |
| 2259 | PHE289 | CG | 49.372 | 33.374 | 43.18 |
| 2260 | PHE289 | CD1 | 50.734 | 33.506 | 42.951 |
| 2261 | PHE289 | CE1 | 51.216 | 33.612 | 41.653 |
| 2262 | PHE289 | CZ | 50.334 | 33.586 | 40.581 |
| 2263 | PHE289 | CE2 | 48.971 | 33.455 | 40.808 |
| 2264 | PHE289 | CD2 | 48.491 | 33.347 | 42.107 |
| 2265 | PHE289 | C | 48.213 | 35.631 | 44.958 |
| 2266 | PHE289 | O | 48.783 | 36.722 | 44.821 |
| 2267 | HIS290 | N | 46.896 | 35.508 | 44.968 |
| 2268 | HIS290 | CA | 46.032 | 36.582 | 44.483 |
| 2269 | HIS290 | CB | 44.721 | 35.971 | 44.008 |
| 2270 | HIS290 | CG | 44.899 | 35.089 | 42.79 |
| 2271 | HIS290 | ND1 | 44.744 | 33.754 | 42.729 |
| 2272 | HIS290 | CE1 | 44.997 | 33.33 | 41.475 |
| 2273 | HIS290 | NE2 | 45.304 | 34.417 | 40.733 |
| 2274 | HIS290 | CD2 | 45.248 | 35.508 | 41.529 |
| 2275 | HIS290 | C | 45.769 | 37.636 | 45.552 |
| 2276 | HIS290 | O | 45.434 | 38.774 | 45.213 |
| 2277 | THR291 | N | 46.122 | 37.341 | 46.791 |
| 2278 | THR291 | CA | 46.007 | 38.343 | 47.849 |
| 2279 | THR291 | CB | 45.971 | 37.653 | 49.206 |
| 2280 | THR291 | OG1 | 44.836 | 36.799 | 49.22 |
| 2281 | THR291 | CG2 | 45.816 | 38.659 | 50.342 |
| 2282 | THR291 | C | 47.175 | 39.316 | 47.782 |
| 2283 | THR291 | O | 46.955 | 40.533 | 47.85 |
| 2284 | LEU292 | N | 48.32 | 38.814 | 47.348 |
| 2285 | LEU292 | CA | 49.477 | 39.68 | 47.138 |
| 2286 | LEU292 | CB | 50.719 | 38.808 | 46.976 |
| 2287 | LEU292 | CG | 51.947 | 39.635 | 46.603 |
| 2288 | LEU292 | CD1 | 52.224 | 40.72 | 47.635 |
| 2289 | LEU292 | CD2 | 53.174 | 38.751 | 46.412 |
| 2290 | LEU292 | C | 49.282 | 40.532 | 45.891 |
| 2291 | LEU292 | O | 49.485 | 41.751 | 45.959 |
| 2292 | PHE293 | N | 48.634 | 39.974 | 44.882 |
| 2293 | PHE293 | CA | 48.42 | 40.743 | 43.652 |
| 2294 | PHE293 | CB | 48.223 | 39.776 | 42.494 |
| 2295 | PHE293 | CG | 49.469 | 38.952 | 42.185 |
| 2296 | PHE293 | CD1 | 49.341 | 37.654 | 41.715 |
| 2297 | PHE293 | CE1 | 50.475 | 36.906 | 41.432 |
| 2298 | PHE293 | CZ | 51.737 | 37.45 | 41.623 |
| 2299 | PHE293 | CE2 | 51.867 | 38.748 | 42.096 |
| 2300 | PHE293 | CD2 | 50.733 | 39.498 | 42.375 |
| 2301 | PHE293 | C | 47.239 | 41.705 | 43.76 |
| 2302 | PHE293 | O | 47.279 | 42.781 | 43.154 |
| 2303 | GLY294 | N | 46.34 | 41.442 | 44.692 |
| 2304 | GLY294 | CA | 45.253 | 42.374 | 44.981 |
| 2305 | GLY294 | C | 45.783 | 43.59 | 45.73 |
| 2306 | GLY294 | O | 45.597 | 44.73 | 45.283 |
| 2307 | ILE295 | N | 46.596 | 43.329 | 46.741 |
| 2308 | ILE295 | CA | 47.18 | 44.399 | 47.558 |
| 2309 | ILE295 | CB | 47.809 | 43.728 | 48.777 |
| 2310 | ILE295 | CG2 | 48.766 | 44.652 | 49.516 |
| 2311 | ILE295 | CG1 | 46.732 | 43.22 | 49.727 |
| 2312 | ILE295 | CD1 | 45.906 | 44.365 | 50.302 |
| 2313 | ILE295 | C | 48.216 | 45.228 | 46.793 |
| 2314 | ILE295 | O | 48.145 | 46.465 | 46.831 |
| 2315 | ALA296 | N | 48.97 | 44.586 | 45.915 |
| 2316 | ALA296 | CA | 49.948 | 45.312 | 45.1 |
| 2317 | ALA296 | CB | 50.926 | 44.304 | 44.509 |
| 2318 | ALA296 | C | 49.266 | 46.088 | 43.977 |
| 2319 | ALA296 | O | 49.598 | 47.259 | 43.747 |
| 2320 | GLY297 | N | 48.18 | 45.526 | 43.471 |
| 2321 | GLY297 | CA | 47.353 | 46.187 | 42.462 |
| 2322 | GLY297 | C | 46.775 | 47.481 | 43.011 |
| 2323 | GLY297 | O | 47.107 | 48.557 | 42.5 |
| 2324 | LEU298 | N | 46.138 | 47.395 | 44.169 |
| 2325 | LEU298 | CA | 45.531 | 48.579 | 44.788 |
| 2326 | LEU298 | CB | 44.743 | 48.153 | 46.02 |
| 2327 | LEU298 | CG | 43.52 | 47.325 | 45.651 |
| 2328 | LEU298 | CD1 | 42.813 | 46.814 | 46.9 |
| 2329 | LEU298 | CD2 | 42.56 | 48.126 | 44.78 |
| 2330 | LEU298 | C | 46.561 | 49.624 | 45.204 |
| 2331 | LEU298 | O | 46.345 | 50.808 | 44.907 |
| 2332 | SER299 | N | 47.742 | 49.205 | 45.629 |
| 2333 | SER299 | CA | 48.766 | 50.182 | 46.009 |
| 2334 | SER299 | CB | 49.912 | 49.465 | 46.705 |
| 2335 | SER299 | OG | 50.832 | 50.461 | 47.127 |
| 2336 | SER299 | C | 49.321 | 50.932 | 44.801 |
| 2337 | SER299 | O | 49.404 | 52.165 | 44.859 |
| 2338 | LEU300 | N | 49.395 | 50.264 | 43.658 |
| 2339 | LEU300 | CA | 49.879 | 50.912 | 42.431 |
| 2340 | LEU300 | CB | 50.432 | 49.827 | 41.507 |
| 2341 | LEU300 | CG | 51.271 | 50.412 | 40.372 |
| 2342 | LEU300 | CD1 | 52.473 | 51.167 | 40.927 |
| 2343 | LEU300 | CD2 | 51.731 | 49.328 | 39.405 |
| 2344 | LEU300 | C | 48.765 | 51.696 | 41.719 |
| 2345 | LEU300 | O | 49.046 | 52.546 | 40.865 |
| 2346 | LEU301 | N | 47.525 | 51.479 | 42.134 |
| 2347 | LEU301 | CA | 46.394 | 52.268 | 41.63 |
| 2348 | LEU301 | CB | 45.132 | 51.411 | 41.668 |
| 2349 | LEU301 | CG | 45.205 | 50.246 | 40.691 |
| 2350 | LEU301 | CD1 | 44.053 | 49.275 | 40.911 |
| 2351 | LEU301 | CD2 | 45.24 | 50.733 | 39.249 |
| 2352 | LEU301 | C | 46.152 | 53.53 | 42.459 |
| 2353 | LEU301 | O | 45.396 | 54.41 | 42.03 |
| 2354 | GLY302 | N | 46.785 | 53.624 | 43.618 |
| 2355 | GLY302 | CA | 46.681 | 54.846 | 44.422 |
| 2356 | GLY302 | C | 46.209 | 54.599 | 45.854 |
| 2357 | GLY302 | O | 45.822 | 55.543 | 46.557 |
| 2358 | GLU303 | N | 46.208 | 53.348 | 46.281 |
| 2359 | GLU303 | CA | 45.812 | 53.052 | 47.661 |
| 2360 | GLU303 | CB | 45.241 | 51.636 | 47.745 |
| 2361 | GLU303 | CG | 44.821 | 51.216 | 49.155 |
| 2362 | GLU303 | CD | 43.859 | 52.219 | 49.782 |
| 2363 | GLU303 | OE1 | 42.661 | 51.998 | 49.714 |
| 2364 | GLU303 | OE2 | 44.356 | 53.158 | 50.394 |
| 2365 | GLU303 | C | 46.999 | 53.24 | 48.603 |
| 2366 | GLU303 | O | 47.681 | 52.273 | 48.962 |
| 2367 | GLU304 | N | 46.991 | 54.409 | 49.227 |
| 2368 | GLU304 | CA | 48.064 | 54.869 | 50.124 |
| 2369 | GLU304 | CB | 47.977 | 56.388 | 50.206 |
| 2370 | GLU304 | CG | 46.613 | 56.843 | 50.713 |
| 2371 | GLU304 | CD | 46.537 | 58.367 | 50.721 |
| 2372 | GLU304 | OE1 | 45.729 | 58.888 | 51.476 |
| 2373 | GLU304 | OE2 | 47.203 | 58.968 | 49.891 |
| 2374 | GLU304 | C | 48.035 | 54.281 | 51.541 |
| 2375 | GLU304 | O | 48.833 | 54.698 | 52.386 |
| 2376 | GLN305 | N | 47.101 | 53.384 | 51.819 |
| 2377 | GLN305 | CA | 47.142 | 52.624 | 53.073 |
| 2378 | GLN305 | CB | 45.746 | 52.098 | 53.385 |
| 2379 | GLN305 | CG | 44.735 | 53.217 | 53.596 |
| 2380 | GLN305 | CD | 43.349 | 52.609 | 53.788 |
| 2381 | GLN305 | OE1 | 43.183 | 51.628 | 54.52 |
| 2382 | GLN305 | NE2 | 42.386 | 53.146 | 53.06 |
| 2383 | GLN305 | C | 48.084 | 51.431 | 52.921 |
| 2384 | GLN305 | O | 48.559 | 50.864 | 53.911 |
| 2385 | ILE306 | N | 48.364 | 51.083 | 51.676 |
| 2386 | ILE306 | CA | 49.302 | 50.013 | 51.367 |
| 2387 | ILE306 | CB | 48.686 | 49.172 | 50.254 |
| 2388 | ILE306 | CG2 | 49.573 | 47.983 | 49.912 |
| 2389 | ILE306 | CG1 | 47.295 | 48.69 | 50.648 |
| 2390 | ILE306 | CD1 | 46.632 | 47.932 | 49.505 |
| 2391 | ILE306 | C | 50.613 | 50.629 | 50.892 |
| 2392 | ILE306 | O | 50.611 | 51.5 | 50.013 |
| 2393 | LYS307 | N | 51.713 | 50.165 | 51.464 |
| 2394 | LYS307 | CA | 53.043 | 50.639 | 51.069 |
| 2395 | LYS307 | CB | 54.093 | 49.846 | 51.84 |
| 2396 | LYS307 | CG | 53.949 | 50.058 | 53.341 |
| 2397 | LYS307 | CD | 55.022 | 49.301 | 54.111 |
| 2398 | LYS307 | CE | 54.863 | 49.495 | 55.614 |
| 2399 | LYS307 | NZ | 55.865 | 48.715 | 56.357 |
| 2400 | LYS307 | C | 53.25 | 50.45 | 49.571 |
| 2401 | LYS307 | O | 52.791 | 49.458 | 48.991 |
| 2402 | PRO308 | N | 53.893 | 51.429 | 48.953 |
| 2403 | PRO308 | CA | 54.059 | 51.432 | 47.498 |
| 2404 | PRO308 | CB | 54.815 | 52.685 | 47.183 |
| 2405 | PRO308 | CG | 55.073 | 53.451 | 48.472 |
| 2406 | PRO308 | CD | 54.442 | 52.631 | 49.585 |
| 2407 | PRO308 | C | 54.81 | 50.192 | 47.042 |
| 2408 | PRO308 | O | 55.782 | 49.77 | 47.681 |
| 2409 | VAL309 | N | 54.282 | 49.559 | 46.012 |
| 2410 | VAL309 | CA | 54.893 | 48.337 | 45.496 |
| 2411 | VAL309 | CB | 53.782 | 47.31 | 45.292 |
| 2412 | VAL309 | CG1 | 52.667 | 47.871 | 44.419 |
| 2413 | VAL309 | CG2 | 54.304 | 45.985 | 44.746 |
| 2414 | VAL309 | C | 55.665 | 48.611 | 44.206 |
| 2415 | VAL309 | O | 55.172 | 49.255 | 43.272 |
| 2416 | ASN310 | N | 56.909 | 48.172 | 44.214 |
| 2417 | ASN310 | CA | 57.79 | 48.283 | 43.055 |
| 2418 | ASN310 | CB | 59.194 | 47.942 | 43.547 |
| 2419 | ASN310 | CG | 60.216 | 47.897 | 42.416 |
| 2420 | ASN310 | OD1 | 60.203 | 46.971 | 41.591 |
| 2421 | ASN310 | ND2 | 61.119 | 48.86 | 42.422 |
| 2422 | ASN310 | C | 57.346 | 47.311 | 41.971 |
| 2423 | ASN310 | O | 57.44 | 46.09 | 42.155 |
| 2424 | PRO311 | N | 57.077 | 47.85 | 40.791 |
| 2425 | PRO311 | CA | 56.422 | 47.09 | 39.715 |
| 2426 | PRO311 | CB | 55.968 | 48.131 | 38.738 |
| 2427 | PRO311 | CG | 56.518 | 49.487 | 39.146 |
| 2428 | PRO311 | CD | 57.241 | 49.269 | 40.462 |
| 2429 | PRO311 | C | 57.309 | 46.056 | 39.004 |
| 2430 | PRO311 | O | 56.789 | 45.237 | 38.241 |
| 2431 | VAL312 | N | 58.594 | 46.018 | 39.316 |
| 2432 | VAL312 | CA | 59.491 | 45.052 | 38.691 |
| 2433 | VAL312 | CB | 60.82 | 45.762 | 38.461 |
| 2434 | VAL312 | CG1 | 61.908 | 44.807 | 37.99 |
| 2435 | VAL312 | CG2 | 60.654 | 46.924 | 37.492 |
| 2436 | VAL312 | C | 59.706 | 43.835 | 39.586 |
| 2437 | VAL312 | O | 59.618 | 42.693 | 39.12 |
| 2438 | PHE313 | N | 59.864 | 44.081 | 40.877 |
| 2439 | PHE313 | CA | 60.187 | 42.992 | 41.809 |
| 2440 | PHE313 | CB | 61.265 | 43.476 | 42.774 |
| 2441 | PHE313 | CG | 62.615 | 43.798 | 42.139 |
| 2442 | PHE313 | CD1 | 62.982 | 45.117 | 41.902 |
| 2443 | PHE313 | CE1 | 64.213 | 45.403 | 41.327 |
| 2444 | PHE313 | CZ | 65.081 | 44.37 | 40.998 |
| 2445 | PHE313 | CE2 | 64.72 | 43.053 | 41.247 |
| 2446 | PHE313 | CD2 | 63.488 | 42.767 | 41.82 |
| 2447 | PHE313 | C | 58.995 | 42.51 | 42.631 |
| 2448 | PHE313 | O | 59.098 | 41.472 | 43.297 |
| 2449 | CYS314 | N | 57.912 | 43.273 | 42.599 |
| 2450 | CYS314 | CA | 56.711 | 43.024 | 43.418 |
| 2451 | CYS314 | CB | 56.06 | 41.705 | 43.004 |
| 2452 | CYS314 | SG | 54.437 | 41.355 | 43.723 |
| 2453 | CYS314 | C | 57.073 | 43.027 | 44.906 |
| 2454 | CYS314 | O | 56.716 | 42.124 | 45.668 |
| 2455 | MET315 | N | 57.829 | 44.043 | 45.29 |
| 2456 | MET315 | CA | 58.271 | 44.201 | 46.681 |
| 2457 | MET315 | CB | 59.775 | 43.939 | 46.766 |
| 2458 | MET315 | CG | 60.14 | 42.489 | 46.474 |
| 2459 | MET315 | SD | 61.893 | 42.094 | 46.663 |
| 2460 | MET315 | CE | 62.08 | 42.498 | 48.415 |
| 2461 | MET315 | C | 57.978 | 45.62 | 47.146 |
| 2462 | MET315 | O | 57.768 | 46.501 | 46.306 |
| 2463 | PRO316 | N | 57.888 | 45.833 | 48.449 |
| 2464 | PRO316 | CA | 57.776 | 47.198 | 48.967 |
| 2465 | PRO316 | CB | 57.767 | 47.065 | 50.457 |
| 2466 | PRO316 | CG | 57.833 | 45.59 | 50.822 |
| 2467 | PRO316 | CD | 57.939 | 44.827 | 49.513 |
| 2468 | PRO316 | C | 58.921 | 48.081 | 48.478 |
| 2469 | PRO316 | O | 60.109 | 47.752 | 48.616 |
| 2470 | GLU317 | N | 58.537 | 49.273 | 48.054 |
| 2471 | GLU317 | CA | 59.458 | 50.231 | 47.437 |
| 2472 | GLU317 | CB | 58.656 | 51.47 | 47.055 |
| 2473 | GLU317 | CG | 59.399 | 52.336 | 46.045 |
| 2474 | GLU317 | CD | 59.31 | 51.675 | 44.678 |
| 2475 | GLU317 | OE1 | 58.257 | 51.118 | 44.404 |
| 2476 | GLU317 | OE2 | 60.271 | 51.741 | 43.921 |
| 2477 | GLU317 | C | 60.562 | 50.662 | 48.391 |
| 2478 | GLU317 | O | 61.735 | 50.555 | 48.021 |
| 2479 | GLU318 | N | 60.232 | 50.779 | 49.669 |
| 2480 | GLU318 | CA | 61.221 | 51.22 | 50.659 |
| 2481 | GLU318 | CB | 60.482 | 51.685 | 51.911 |
| 2482 | GLU318 | CG | 59.622 | 50.584 | 52.522 |
| 2483 | GLU318 | CD | 58.833 | 51.135 | 53.706 |
| 2484 | GLU318 | OE1 | 58.506 | 50.346 | 54.58 |
| 2485 | GLU318 | OE2 | 58.427 | 52.284 | 53.618 |
| 2486 | GLU318 | C | 62.265 | 50.155 | 51.021 |
| 2487 | GLU318 | O | 63.376 | 50.528 | 51.412 |
| 2488 | VAL319 | N | 62.031 | 48.904 | 50.652 |
| 2489 | VAL319 | CA | 63.021 | 47.866 | 50.927 |
| 2490 | VAL319 | CB | 62.3 | 46.53 | 51.068 |
| 2491 | VAL319 | CG1 | 63.288 | 45.382 | 51.243 |
| 2492 | VAL319 | CG2 | 61.314 | 46.574 | 52.228 |
| 2493 | VAL319 | C | 64.019 | 47.81 | 49.78 |
| 2494 | VAL319 | O | 65.232 | 47.726 | 50.015 |
| 2495 | LEU320 | N | 63.543 | 48.196 | 48.607 |
| 2496 | LEU320 | CA | 64.409 | 48.232 | 47.431 |
| 2497 | LEU320 | CB | 63.552 | 48.006 | 46.197 |
| 2498 | LEU320 | CG | 62.901 | 46.633 | 46.277 |
| 2499 | LEU320 | CD1 | 61.914 | 46.423 | 45.144 |
| 2500 | LEU320 | CD2 | 63.951 | 45.529 | 46.28 |
| 2501 | LEU320 | C | 65.161 | 49.554 | 47.358 |
| 2502 | LEU320 | O | 66.308 | 49.585 | 46.895 |
| 2503 | GLN321 | N | 64.645 | 50.546 | 48.062 |
| 2504 | GLN321 | CA | 65.392 | 51.787 | 48.265 |
| 2505 | GLN321 | CB | 64.432 | 52.844 | 48.791 |
| 2506 | GLN321 | CG | 63.324 | 53.166 | 47.799 |
| 2507 | GLN321 | CD | 62.241 | 53.956 | 48.524 |
| 2508 | GLN321 | OE1 | 61.048 | 53.849 | 48.211 |
| 2509 | GLN321 | NE2 | 62.662 | 54.652 | 49.565 |
| 2510 | GLN321 | C | 66.498 | 51.591 | 49.299 |
| 2511 | GLN321 | O | 67.619 | 52.064 | 49.084 |
| 2512 | ARG322 | N | 66.259 | 50.719 | 50.266 |
| 2513 | ARG322 | CA | 67.257 | 50.455 | 51.309 |
| 2514 | ARG322 | CB | 66.561 | 49.715 | 52.446 |
| 2515 | ARG322 | CG | 67.543 | 49.308 | 53.538 |
| 2516 | ARG322 | CD | 66.855 | 48.523 | 54.649 |
| 2517 | ARG322 | NE | 67.832 | 48.089 | 55.66 |
| 2518 | ARG322 | CZ | 67.913 | 48.623 | 56.881 |
| 2519 | ARG322 | NH1 | 67.07 | 49.591 | 57.245 |
| 2520 | ARG322 | NH2 | 68.831 | 48.179 | 57.742 |
| 2521 | ARG322 | C | 68.423 | 49.612 | 50.793 |
| 2522 | ARG322 | O | 69.581 | 49.875 | 51.139 |
| 2523 | VAL323 | N | 68.141 | 48.714 | 49.861 |
| 2524 | VAL323 | CA | 69.218 | 47.926 | 49.251 |
| 2525 | VAL323 | CB | 68.723 | 46.508 | 48.984 |
| 2526 | VAL323 | CG1 | 68.421 | 45.787 | 50.293 |
| 2527 | VAL323 | CG2 | 67.505 | 46.492 | 48.07 |
| 2528 | VAL323 | C | 69.771 | 48.564 | 47.973 |
| 2529 | VAL323 | O | 70.749 | 48.053 | 47.413 |
| 2530 | ASN324 | N | 69.228 | 49.717 | 47.604 |
| 2531 | ASN324 | CA | 69.642 | 50.478 | 46.413 |
| 2532 | ASN324 | CB | 71.082 | 50.964 | 46.579 |
| 2533 | ASN324 | CG | 71.19 | 51.955 | 47.736 |
| 2534 | ASN324 | OD1 | 70.782 | 53.116 | 47.613 |
| 2535 | ASN324 | ND2 | 71.809 | 51.509 | 48.818 |
| 2536 | ASN324 | C | 69.498 | 49.667 | 45.13 |
| 2537 | ASN324 | O | 70.41 | 49.614 | 44.296 |
| 2538 | VAL325 | N | 68.321 | 49.093 | 44.951 |
| 2539 | VAL325 | CA | 68.017 | 48.324 | 43.742 |
| 2540 | VAL325 | CB | 67.85 | 46.841 | 44.078 |
| 2541 | VAL325 | CG1 | 67.643 | 46.023 | 42.809 |
| 2542 | VAL325 | CG2 | 69.065 | 46.3 | 44.823 |
| 2543 | VAL325 | C | 66.752 | 48.894 | 43.107 |
| 2544 | VAL325 | O | 65.658 | 48.32 | 43.17 |
| 2545 | GLN326 | N | 66.921 | 50.071 | 42.53 |
| 2546 | GLN326 | CA | 65.801 | 50.776 | 41.899 |
| 2547 | GLN326 | CB | 65.739 | 52.189 | 42.474 |
| 2548 | GLN326 | CG | 65.343 | 52.191 | 43.948 |
| 2549 | GLN326 | CD | 63.876 | 51.794 | 44.104 |
| 2550 | GLN326 | OE1 | 63.514 | 51.013 | 44.992 |
| 2551 | GLN326 | NE2 | 63.04 | 52.375 | 43.261 |
| 2552 | GLN326 | C | 65.941 | 50.843 | 40.381 |
| 2553 | GLN326 | O | 66.745 | 51.617 | 39.85 |
| 2554 | PRO327 | N | 65.116 | 50.063 | 39.702 |
| 2555 | PRO327 | CA | 65.046 | 50.094 | 38.238 |
| 2556 | PRO327 | CB | 64.162 | 48.944 | 37.87 |
| 2557 | PRO327 | CG | 63.557 | 48.354 | 39.133 |
| 2558 | PRO327 | CD | 64.142 | 49.142 | 40.289 |
| 2559 | PRO327 | C | 64.466 | 51.413 | 37.732 |
| 2560 | PRO327 | O | 63.543 | 51.978 | 38.334 |
| 2561 | GLU328 | N | 65.004 | 51.89 | 36.624 |
| 2562 | GLU328 | CA | 64.529 | 53.157 | 36.052 |
| 2563 | GLU328 | CB | 65.71 | 53.912 | 35.454 |
| 2564 | GLU328 | CG | 65.288 | 55.275 | 34.91 |
| 2565 | GLU328 | CD | 66.489 | 55.969 | 34.28 |
| 2566 | GLU328 | OE1 | 67.6 | 55.584 | 34.621 |
| 2567 | GLU328 | OE2 | 66.282 | 56.888 | 33.5 |
| 2568 | GLU328 | C | 63.491 | 52.908 | 34.966 |
| 2569 | GLU328 | O | 63.842 | 52.713 | 33.8 |
| 2570 | LEU329 | N | 62.228 | 53.029 | 35.338 |
| 2571 | LEU329 | CA | 61.119 | 52.793 | 34.399 |
| 2572 | LEU329 | CB | 59.807 | 52.832 | 35.175 |
| 2573 | LEU329 | CG | 59.845 | 51.956 | 36.426 |
| 2574 | LEU329 | CD1 | 58.562 | 52.115 | 37.232 |
| 2575 | LEU329 | CD2 | 60.092 | 50.487 | 36.099 |
| 2576 | LEU329 | C | 61.092 | 53.866 | 33.31 |
| 2577 | LEU329 | O | 61.756 | 54.903 | 33.446 |
| 2578 | VAL330 | N | 60.407 | 53.582 | 32.214 |
| 2579 | VAL330 | CA | 60.291 | 54.567 | 31.129 |
| 2580 | VAL330 | CB | 59.554 | 53.943 | 29.945 |
| 2581 | VAL330 | CG1 | 59.371 | 54.932 | 28.796 |
| 2582 | VAL330 | CG2 | 60.245 | 52.685 | 29.442 |
| 2583 | VAL330 | C | 59.51 | 55.78 | 31.621 |
| 2584 | VAL330 | O | 58.394 | 55.649 | 32.139 |
| 2585 | SER331 | N | 60.135 | 56.939 | 31.528 |
| 2586 | SER331 | CA | 59.479 | 58.179 | 31.939 |
| 2587 | SER331 | CB | 60.269 | 58.798 | 33.082 |
| 2588 | SER331 | OG | 59.633 | 60.024 | 33.412 |
| 2589 | SER331 | C | 59.404 | 59.166 | 30.781 |
| 2590 | SER331 | O | 60.341 | 59.197 | 29.998 |
| 2591 | SER331 | OXT | 58.428 | 59.902 | 30.728 |

What is claimed is:

1. A method of inducing apoptosis in a eukaryotic cell, the method comprising contacting the cell with an agent that is a RabGGT inhibitor.

2. The method of claim 1, wherein the RabGGT inhibitor reduces the level of RabGGT mRNA in the cell.

3. The method of claim 1, wherein the RabGGT inhibitor is an interfering RNA.

4. The method of claim 1, wherein the RabGGT inhibitor reduces the level of RabGGT protein in the cell.

5. The method of claim 1, wherein the RabGGT inhibitor inhibits RabGGT enzymatic activity.

6. The method of claim 5, wherein the RabGGT inhibitor is a benzodiazapine compound.

7. The method of claim 5, wherein the RabGGT inhibitor is a tetrahydroquinoline compound.

8. The method of claim 1, wherein the agent does not substantially inhibit farnesyl transferase activity.

9. A method of inhibiting tumor growth in an individual having a tumor, the method comprising:
identifying a compound that is a RabGGT inhibitor;
testing the ability of the compound to modulate farnesyl transferase (FT) activity;
modifying the compound, wherein the modified compound exhibits reduced modulation of FT activity compared to the unmodified compound, wherein inhibition of RabGGT is retained; and
administering to the individual an effective amount of an agent that is a RabGGT inhibitor.

10. The method of claim 9, wherein the RabGGT inhibitor reduces the level of RabGGT mRNA in the tumor.

11. The method of claim 9, wherein the RabGGT inhibitor is an interfering RNA.

12. The method of claim 9, wherein the RabGGT inhibitor reduces the level of RabGGT protein in the tumor.

13. The method of claim 9, wherein the RabGGT inhibitor inhibits RabGGT enzymatic activity.

14. The method of claim 13, wherein the RabGGT inhibitor is a benzodiazapine compound.

15. The method of claim 13, wherein the RabGGT inhibitor is a tetrahydroquinoline compound.

16. The method of claim 9, wherein the agent does not substantially inhibit famesyl transferase activity.

17. A method of determining the susceptibility of a tumor to treatment with a RabGGT inhibitor, the method comprising detecting a level of RabGGT in the tumor, wherein a level of RabGGT that is elevated compared to a normal cell of the same tissue type indicates that the tumor is susceptible to treatment with a RabGGT inhibitor.

18. A method of identifying an agent that selectively modulates RabGGT enzymatic activity, the method comprising;
determining the effect, if any, of the agent on enzymatic activity of RabGGT; and
determining the effect, if any, of the agent on enzymatic activity of farnesyl transferase;
wherein an increase or decrease of enzymatic activity of RabGGT of at least about 15% compared to the enzymatic activity of RabGGT in the absence of the agent, and a reduction of enzymatic activity of farnesyl transferase of less than about 10% compared to the enzymatic activity of famesyl transferase in the absence of the agent, indicates that the agent is a selective modulator of RabGGT enzymatic activity.

19. An agent identified by the method of claim 18.

20. A method of identifying an agent that modulates RabGGT enzymatic activity and modulates apoptosis, the method comprising:
determining the effect, if any, of the agent on RabGGT enzymatic activity; and
determining the effect, if any, of the agent on apoptosis in a eukaryotic cell,
wherein an increase or decrease of enzymatic activity of RabGGT of at least about 15% compared to the enzymatic activity of RabGGT in the absence of the agent, and wherein an increase or decrease in apoptosis of at least about 15% compared to the level of apoptosis in the absence of the agent indicates that the agent modulates RabGGT enzymatic activity and apoptosis.

21. A database comprising:
a machine-readable data storage medium comprising a data storage material encoded with machine-readable data, wherein said data comprises the three-dimensional coordinates of a subset of the atoms in a RabGGT polypeptide.

22. A computer for producing a three-dimensional representation of a RabGGT protein, wherein said computer comprises:
a machine-readable data storage medium comprising a data storage material encoded with machine-readable data, wherein said data comprises the three-dimensional coordinates of a subset of the atoms in RabGGT polypeptide;
a working memory for storing instructions for processing said machine-readable data;
a central-processing unit coupled to said working memory and to said machine-readable data storage medium for processing said machine readable data into said three-dimensional representation; and
a display coupled to said central-processing unit for displaying said three-dimensional representation.

23. The computer of claim 22, wherein said RabGGT polypeptide is complexed with a Rab protein.

24. The computer of claim 22, wherein said RabGGT polypeptide is bound to an agent.

25. The computer of claim 24, wherein said agent is an inhibitor of RabGGT enzymatic activity.

26. A computer-assisted method for identifying potential modulators of apoptosis, using a programmed computer comprising a processor, a data storage system, an input device, and an output device, comprising the steps of:
(a) inputting into the programmed computer through said input device data comprising the three-dimensional coordinates of a subset of the atoms in a RabGGT enzyme, thereby generating a criteria data set;
(b) comparing, using said processor, said criteria data set to a computer database of chemical structures stored in said computer data storage system;
(c) selecting from said database, using computer methods, chemical structures having a portion that is structurally similar to said criteria data set;
(d) outputting to said output device the selected chemical structures having a portion similar to said criteria data set.

27. A compound having a chemical structure selected using the method of claim 26.

28. A method of identifying an agent that modulates a binding event between a RabGGT polypeptide and a second polypeptide or polypeptide complex, the method comprising:
contacting the agent with a sample comprising a RabGGT polypeptide and a second polypeptide; and
determining the effect, if any, of the test agent on the binding between the RabGGT polyeptide and the second polypeptide or polypeptide complex.

29. The method of claim 28, wherein the second polypeptide is a Rab polypeptide.

30. The method of claim 28, wherein the polypeptide complex is a Rab/REP complex.

31. The method of claim 28, wherein said determining is performed using a method selected from a FRET assay, a BRET assay, a fluorescence quenching assay; a fluorescence anisotropy assay; an immunological assay; and an assay involving binding of a detectably labeled protein to an immobilized protein.

32. A method of identifying an agent that induces apoptosis and/or inhibits cell proliferation comprising:
a) screening a test agent in an assay system that detects changes in RabGGT level or activity,
b) identifying a test agent that reduces RabGGT levels or activity in said assay system, and
c) determining whether the test agent identified in (b) induces apoptosis in a cell and/or inhibits cell proliferation.

33. The method of claim 32 wherein the assay system is a high-throughput screening (HTS) system that detects changes in RabGGT enzymatic activity.

34. A method of identifying a clinical compound for treatment of disorders associated with undesired or uncontrolled cell proliferation comprising:
a) performing the method of claim 32 to identify an agent that induces apoptosis and/or inhibits cell proliferation,
b) using said agent as a lead compound to design and synthesize analog compounds, and
c) selecting an analog compound having favorable properties for use as a clinical compound.

35. A kit comprising a clinical compound identified according to the method of claim 34 and instructions for administering the clinical compound to a patient afflicted with a disorder associated with undesired or uncontrolled cell proliferation.

36. A method of inducing apoptosis in a cell comprising contacting the cell with the clinical compound identified by the method of claim 34.

37. The method of claim 1, wherein the RabGGT inhibitor is an antibody.
